SEQUENCE LISTING (192 sequences)

SEQ ID NO: 1 - protein, 12 aa, synthetic construct
  SITE 2: note = Aib
HXEGTFTSDYSS

SEQ ID NO: 2 - protein, 12 aa, synthetic construct
YLEEQAAKEFIA

SEQ ID NO: 3 - protein, 11 aa, synthetic construct
WLVKGGGGGGG

SEQ ID NO: 4 - protein, 12 aa, synthetic construct
SGGGGSGGGGSK

SEQ ID NO: 5 - protein, 8 aa, synthetic construct
AWLVKGGG

SEQ ID NO: 6 - protein, 16 aa, synthetic construct
GGGGSGGGGSGGGGSK

SEQ ID NO: 7 - protein, 47 aa, synthetic construct
  SITE 2: note = Aib
HXEGTFTSDYSSYLEEQAAKEFIAWLVKGGGGGGGSGGGGSGGGGSK

SEQ ID NO: 8 - protein, 23 aa, synthetic construct
WLVKGGGGGGGSGGGGSGGGGSK

SEQ ID NO: 9 - protein, 24 aa, synthetic construct
AWLVKGGGGGGGSGGGGSGGGGSK

SEQ ID NO: 10 - protein, 34 aa, synthetic construct
LEEQAAKEFIAWLVKGGGGGGGSGGGGSGGGGSK

SEQ ID NO: 11 - protein, 13 aa, synthetic construct
  SITE 2: note = Aib
HXEGTFTSDYSSY

SEQ ID NO: 12 - protein, 10 aa, synthetic construct
LEEQAAKEFI

SEQ ID NO: 13 - protein, 12 aa, synthetic construct
AWLVKGGGGGGG

SEQ ID NO: 14 - protein, 35 aa, synthetic construct
YLEEQAAKEFIAWLVKGGGGGGGSGGGGSGGGGSK

SEQ ID NO: 15 - protein, 23 aa, synthetic construct
  SITE 2: note = Aib
HXEGTFTSDYSSYLEEQAAKEFI

SEQ ID NO: 16 - protein, 11 aa, synthetic construct
YLEEQAAKEFI

SEQ ID NO: 17 - protein, 11 aa, synthetic construct
LEEQAAKEFIA

SEQ ID NO: 18 - protein, 12 aa, synthetic construct
  SITE 1: note = Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OMpe (O-3-methylpent-3-yl) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
HXEGTFTSDYSS

SEQ ID NO: 19 - protein, 12 aa, synthetic construct
  SITE 1: note = Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OMpe (O-3-methylpent-3-yl) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
HXEGTFTSDYSS

SEQ ID NO: 20 - protein, 13 aa, synthetic construct
  SITE 1: note = Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Side chain of Ser is protected with a tert-butyl (tBu) protecting group
  SITE 13: note = Side chain of Tyr is protected with a tert-butyl (tBu) protecting group
HXEGTFTSDYSSY

SEQ ID NO: 21 - protein, 13 aa, synthetic construct
  SITE 1: note = Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Side chain of Ser is protected with a tert-butyl (tBu) protecting group
  SITE 13: note = Side chain of Tyr is protected with a tert-butyl (tBu) protecting group
HXEGTFTSDYSSY

SEQ ID NO: 22 - protein, 23 aa, synthetic construct
  SITE 1: note = Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
HXEGTFTSDYSSYLEEQAAKEFI

SEQ ID NO: 23 - protein, 12 aa, synthetic construct
  SITE 1: note = N-terminus of His is protected; Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OMpe (O-3-methylpent-3-yl) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
HXEGTFTSDYSS

SEQ ID NO: 24 - protein, 12 aa, synthetic construct
  SITE 1: note = N-terminus of His is protected; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OMpe (O-3-methylpent-3-yl) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
HXEGTFTSDYSS

SEQ ID NO: 25 - protein, 13 aa, synthetic construct
  SITE 1: note = N-terminus of His is protected; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Side chain of Ser is protected with a tert-butyl (tBu) protecting group
  SITE 13: note = Side chain of Tyr is protected with a tert-butyl (tBu) protecting group
HXEGTFTSDYSSY

SEQ ID NO: 26 - protein, 13 aa, synthetic construct
  SITE 1: note = N-terminus of His is protected; Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Side chain of Ser is protected with a tert-butyl (tBu) protecting group
  SITE 13: note = Side chain of Tyr is protected with a tert-butyl (tBu) protecting group
HXEGTFTSDYSSY

SEQ ID NO: 27 - protein, 23 aa, synthetic construct
  SITE 1: note = N-terminus of His is protected; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
HXEGTFTSDYSSYLEEQAAKEFI

SEQ ID NO: 28 - protein, 12 aa, synthetic construct
  SITE 1: note = Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
YLEEQAAKEFIA

SEQ ID NO: 29 - protein, 10 aa, synthetic construct
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
LEEQAAKEFI

SEQ ID NO: 30 - protein, 11 aa, synthetic construct
  SITE 1: note = Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
YLEEQAAKEFI

SEQ ID NO: 31 - protein, 11 aa, synthetic construct
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
LEEQAAKEFIA

SEQ ID NO: 32 - protein, 11 aa, synthetic construct
  SITE 1: note = Side chain of Trp is protected with a Boc (tert-butyloxycarbonyl) protecting group
  SITE 4: note = Side chain of Lys is protected with a Boc (tert-butyloxycarbonyl) protecting group
WLVKGGGGGGG

SEQ ID NO: 33 - protein, 12 aa, synthetic construct
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
AWLVKGGGGGGG

SEQ ID NO: 34 - protein, 8 aa, synthetic construct
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
AWLVKGGG

SEQ ID NO: 35 - protein, 8 aa, synthetic construct
  SITE 2: note = Side chain of Trp is protected with a Boc (tert-butyloxycarbonyl) protecting group
AWLVKGGG

SEQ ID NO: 36 - protein, 12 aa, synthetic construct
  SITE 1: note = N-terminus of Tyr is protected; Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
YLEEQAAKEFIA

SEQ ID NO: 37 - protein, 10 aa, synthetic construct
  SITE 1: note = N-terminus of Leu is protected
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
LEEQAAKEFI

SEQ ID NO: 38 - protein, 11 aa, synthetic construct
  SITE 1: note = N-terminus of Tyr is protected; Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
YLEEQAAKEFI

SEQ ID NO: 39 - protein, 11 aa, synthetic construct
  SITE 1: note = N-terminus of Leu is protected
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
LEEQAAKEFIA

SEQ ID NO: 40 - protein, 11 aa, synthetic construct
  SITE 1: note = N-terminus of Trp is protected; Side chain of Trp is protected with a Boc (tert-butyloxycarbonyl) protecting group
  SITE 4: note = Side chain of Lys is protected with a Boc (tert-butyloxycarbonyl) protecting group
WLVKGGGGGGG

SEQ ID NO: 41 - protein, 12 aa, synthetic construct
  SITE 1: note = N-terminus of Ala is protected
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
AWLVKGGGGGGG

SEQ ID NO: 42 - protein, 8 aa, synthetic construct
  SITE 1: note = N-terminus of Ala is protected
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
AWLVKGGG

SEQ ID NO: 43 - protein, 8 aa, synthetic construct
  SITE 1: note = N-terminus of Ala is protected
  SITE 2: note = Side chain of Trp is protected with a Boc (tert-butyloxycarbonyl) protecting group
AWLVKGGG

SEQ ID NO: 44 - protein, 12 aa, synthetic construct
  SITE 1: note = Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
  SITE 12: note = C-terminus of Ala is protected
YLEEQAAKEFIA

SEQ ID NO: 45 - protein, 10 aa, synthetic construct
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
  SITE 10: note = C-terminus of Ile is protected
LEEQAAKEFI

SEQ ID NO: 46 - protein, 11 aa, synthetic construct
  SITE 1: note = Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
  SITE 11: note = C-terminus of Ile is protected
YLEEQAAKEFI

SEQ ID NO: 47 - protein, 11 aa, synthetic construct
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
  SITE 11: note = C-terminus of Ala is protected
LEEQAAKEFIA

SEQ ID NO: 48 - protein, 11 aa, synthetic construct
  SITE 1: note = Side chain of Trp is protected with a Boc (tert-butyloxycarbonyl) protecting group
  SITE 4: note = Side chain of Lys is protected with a Boc (tert-butyloxycarbonyl) protecting group
  SITE 11: note = C-terminus of Gly is protected
WLVKGGGGGGG

SEQ ID NO: 49 - protein, 12 aa, synthetic construct
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 12: note = C-terminus of Gly is protected
AWLVKGGGGGGG

SEQ ID NO: 50 - protein, 8 aa, synthetic construct
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 8: note = C-terminus of Gly is protected
AWLVKGGG

SEQ ID NO: 51 - protein, 8 aa, synthetic construct
  SITE 2: note = Side chain of Trp is protected with a Boc (tert-butyloxycarbonyl) protecting group
  SITE 8: note = C-terminus of Gly is protected
AWLVKGGG

SEQ ID NO: 52 - protein, 12 aa, synthetic construct
  SITE 1: note = N-terminus of Tyr is protected with a Fmoc (9-fluorenylmethyloxycarbonyl) protecting group; Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
YLEEQAAKEFIA

SEQ ID NO: 53 - protein, 10 aa, synthetic construct
  SITE 1: note = N-terminus of Leu is protected with a Fmoc (9-fluorenylmethyloxycarbonyl) protecting group
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
LEEQAAKEFI

SEQ ID NO: 54 - protein, 11 aa, synthetic construct
  SITE 1: note = N-terminus of Tyr is protected with a Fmoc (9-fluorenylmethyloxycarbonyl) protecting group; Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
YLEEQAAKEFI

SEQ ID NO: 55 - protein, 11 aa, synthetic construct
  SITE 1: note = N-terminus of Leu is protected with a Fmoc (9-fluorenylmethyloxycarbonyl) protecting group
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
LEEQAAKEFIA

SEQ ID NO: 56 - protein, 11 aa, synthetic construct
  SITE 1: note = N-terminus of Trp is protected with a Fmoc (9-fluorenylmethyloxycarbonyl) protecting group; Side chain of Trp is protected with a Boc (tert-butyloxycarbonyl) protecting group
  SITE 4: note = Side chain of Lys is protected with a Boc (tert-butyloxycarbonyl) protecting group
WLVKGGGGGGG

SEQ ID NO: 57 - protein, 12 aa, synthetic construct
  SITE 1: note = N-terminus of Ala is protected with a Fmoc (9-fluorenylmethyloxycarbonyl) protecting group
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
AWLVKGGGGGGG

SEQ ID NO: 58 - protein, 8 aa, synthetic construct
  SITE 1: note = N-terminus of Ala is protected with a Fmoc (9-fluorenylmethyloxycarbonyl) protecting group
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
AWLVKGGG

SEQ ID NO: 59 - protein, 8 aa, synthetic construct
  SITE 1: note = N-terminus of Ala is protected with a Trt (trityl) protecting group
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
AWLVKGGG

SEQ ID NO: 60 - protein, 8 aa, synthetic construct
  SITE 1: note = N-terminus of Ala is protected with a Fmoc (9-fluorenylmethyloxycarbonyl) protecting group
  SITE 2: note = Side chain of Trp is protected with a Boc (tert-butyloxycarbonyl) protecting group
AWLVKGGG

SEQ ID NO: 61 - protein, 12 aa, synthetic construct
  SITE 1: note = Side chain of Ser is protected with a tert-butyl (tBu) protecting group
  SITE 6: note = Side chain of Ser is protected with a tert-butyl (tBu) protecting group
  SITE 11: note = Side chain of Ser is protected with a tert-butyl (tBu) protecting group
  SITE 12: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
SGGGGSGGGGSK

SEQ ID NO: 62 - protein, 16 aa, synthetic construct
  SITE 5: note = Ser is protected as a dimethylated pseudoproline (Psi(Me,Me)pro) moiety in which the oxazolidine is derived from Ser
  SITE 10: note = Ser is protected as a dimethylated pseudoproline (Psi(Me,Me)pro) moiety in which the oxazolidine is derived from Ser
  SITE 15: note = Ser is protected as a dimethylated pseudoproline (Psi(Me,Me)pro) moiety in which the oxazolidine is derived from Ser
  SITE 16: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
GGGGSGGGGSGGGGSK

SEQ ID NO: 63 - protein, 16 aa, synthetic construct
  SITE 5: note = Side chain of Ser is protected with a tert-butyl (tBu) protecting group
  SITE 10: note = Side chain of Ser is protected with a tert-butyl (tBu) protecting group
  SITE 15: note = Side chain of Ser is protected with a tert-butyl (tBu) protecting group
  SITE 16: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
GGGGSGGGGSGGGGSK

SEQ ID NO: 64 - protein, 24 aa, synthetic construct
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Side chain of Ser is protected with a tert-butyl (tBu) protecting group
  SITE 18: note = Side chain of Ser is protected with a tert-butyl (tBu) protecting group
  SITE 23: note = Side chain of Ser is protected with a tert-butyl (tBu) protecting group
  SITE 24: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
AWLVKGGGGGGGSGGGGSGGGGSK

SEQ ID NO: 65 - protein, 34 aa, synthetic construct; identical to SEQ ID NO: 10
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
  SITE 12: note = Side chain of Trp is protected with a Boc (tert-butyloxycarbonyl) protecting group
  SITE 15: note = Side chain of Lys is protected with a Boc (tert-butyloxycarbonyl) protecting group
  SITE 23: note = Side chain of Ser is protected with a tert-butyl (tBu) protecting group
  SITE 28: note = Side chain of Ser is protected with a tert-butyl (tBu) protecting group
  SITE 33: note = Side chain of Ser is protected with a tert-butyl (tBu) protecting group
  SITE 34: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group

SEQ ID NO: 66 - protein, 23 aa, synthetic construct
  SITE 1: note = Side chain of Trp is protected with a Boc (tert-butyloxycarbonyl) protecting group
  SITE 4: note = Side chain of Lys is protected with a Boc (tert-butyloxycarbonyl) protecting group
  SITE 12: note = Side chain of Ser is protected with a tert-butyl (tBu) protecting group
  SITE 17: note = Side chain of Ser is protected with a tert-butyl (tBu) protecting group
  SITE 22: note = Side chain of Ser is protected with a tert-butyl (tBu) protecting group
  SITE 23: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
WLVKGGGGGGGSGGGGSGGGGSK

SEQ ID NO: 67 - protein, 35 aa, synthetic construct; identical to SEQ ID NO: 14
  SITE 1: note = Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
  SITE 13: note = Side chain of Trp is protected with a Boc (tert-butyloxycarbonyl) protecting group
  SITE 16: note = Side chain of Lys is protected with a Boc (tert-butyloxycarbonyl) protecting group
  SITE 24: note = Side chain of Ser is protected with a tert-butyl (tBu) protecting group
  SITE 29: note = Side chain of Ser is protected with a tert-butyl (tBu) protecting group
  SITE 34: note = Side chain of Ser is protected with a tert-butyl (tBu) protecting group
  SITE 35: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group

SEQ ID NO: 68 - protein, 12 aa, synthetic construct
  SITE 1: note = N-terminus of Ser is protected; Side chain of Ser is protected with a tert-butyl (tBu) protecting group
  SITE 6: note = Side chain of Ser is protected with a tert-butyl (tBu) protecting group
  SITE 11: note = Side chain of Ser is protected with a tert-butyl (tBu) protecting group
  SITE 12: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
SGGGGSGGGGSK

SEQ ID NO: 69 - protein, 16 aa, synthetic construct
  SITE 1: note = N-terminus of Gly is protected
  SITE 5: note = Ser is protected as a dimethylated pseudoproline (Psi(Me,Me)pro) moiety in which the oxazolidine is derived from Ser
  SITE 10: note = Ser is protected as a dimethylated pseudoproline (Psi(Me,Me)pro) moiety in which the oxazolidine is derived from Ser
  SITE 15: note = Ser is protected as a dimethylated pseudoproline (Psi(Me,Me)pro) moiety in which the oxazolidine is derived from Ser
  SITE 16: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
GGGGSGGGGSGGGGSK

SEQ ID NO: 70 - protein, 16 aa, synthetic construct
  SITE 1: note = N-terminus of Gly is protected
  SITE 5: note = Side chain of Ser is protected with a tert-butyl (tBu) protecting group
  SITE 10: note = Side chain of Ser is protected with a tert-butyl (tBu) protecting group
  SITE 15: note = Side chain of Ser is protected with a tert-butyl (tBu) protecting group
  SITE 16: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
GGGGSGGGGSGGGGSK

SEQ ID NO: 71 - protein, 24 aa, synthetic construct
  SITE 1: note = N-terminus of Ala is protected
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Side chain of Ser is protected with a tert-butyl (tBu) protecting group
  SITE 18: note = Side chain of Ser is protected with a tert-butyl (tBu) protecting group
  SITE 23: note = Side chain of Ser is protected with a tert-butyl (tBu) protecting group
  SITE 24: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
AWLVKGGGGGGGSGGGGSGGGGSK

SEQ ID NO: 72 - protein, 24 aa, synthetic construct
  SITE 1: note = N-terminus of Ala is protected
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Ser is protected as a dimethylated pseudoproline (Psi(Me,Me)pro) moiety in which the oxazolidine is derived from Ser
  SITE 18: note = Ser is protected as a dimethylated pseudoproline (Psi(Me,Me)pro) moiety in which the oxazolidine is derived from Ser
  SITE 23: note = Ser is protected as a dimethylated pseudoproline (Psi(Me,Me)pro) moiety in which the oxazolidine is derived from Ser
  SITE 24: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
AWLVKGGGGGGGSGGGGSGGGGSK

SEQ ID NO: 73 - protein, 34 aa, synthetic construct; identical to SEQ ID NO: 10
  SITE 1: note = N-terminus of Leu is protected
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
  SITE 12: note = Side chain of Trp is protected with a Boc (tert-butyloxycarbonyl) protecting group
  SITE 15: note = Side chain of Lys is protected with a Boc (tert-butyloxycarbonyl) protecting group
  SITE 23: note = Side chain of Ser is protected with a tert-butyl (tBu) protecting group
  SITE 28: note = Side chain of Ser is protected with a tert-butyl (tBu) protecting group
  SITE 33: note = Side chain of Ser is protected with a tert-butyl (tBu) protecting group
  SITE 34: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group

SEQ ID NO: 74 - protein, 23 aa, synthetic construct
  SITE 1: note = N-terminus of Trp is protected; Side chain of Trp is protected with a Boc (tert-butyloxycarbonyl) protecting group
  SITE 4: note = Side chain of Lys is protected with a Boc (tert-butyloxycarbonyl) protecting group
  SITE 12: note = Side chain of Ser is protected with a tert-butyl (tBu) protecting group
  SITE 17: note = Side chain of Ser is protected with a tert-butyl (tBu) protecting group
  SITE 22: note = Side chain of Ser is protected with a tert-butyl (tBu) protecting group
  SITE 23: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
WLVKGGGGGGGSGGGGSGGGGSK

SEQ ID NO: 75 - protein, 35 aa, synthetic construct; identical to SEQ ID NO: 14
  SITE 1: note = N-terminus of Tyr is protected; Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
  SITE 13: note = Side chain of Trp is protected with a Boc (tert-butyloxycarbonyl) protecting group
  SITE 16: note = Side chain of Lys is protected with a Boc (tert-butyloxycarbonyl) protecting group
  SITE 24: note = Side chain of Ser is protected with a tert-butyl (tBu) protecting group
  SITE 29: note = Side chain of Ser is protected with a tert-butyl (tBu) protecting group
  SITE 34: note = Side chain of Ser is protected with a tert-butyl (tBu) protecting group
  SITE 35: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group

SEQ ID NO: 76 - protein, 12 aa, synthetic construct
  SITE 1: note = N-terminus of Ser is protected with a Fmoc (9-fluorenylmethyloxycarbonyl) protecting group; Side chain of Ser is protected with a tert-butyl (tBu) protecting group
  SITE 6: note = Side chain of Ser is protected with a tert-butyl (tBu) protecting group
  SITE 11: note = Side chain of Ser is protected with a tert-butyl (tBu) protecting group
  SITE 12: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
SGGGGSGGGGSK

SEQ ID NO: 77 - protein, 16 aa, synthetic construct
  SITE 1: note = N-terminus of Gly is protected with a Fmoc (9-fluorenylmethyloxycarbonyl) protecting group
  SITE 5: note = Side chain of Ser is protected with a tert-butyl (tBu) protecting group
  SITE 10: note = Side chain of Ser is protected with a tert-butyl (tBu) protecting group
  SITE 15: note = Side chain of Ser is protected with a tert-butyl (tBu) protecting group
  SITE 16: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
GGGGSGGGGSGGGGSK

SEQ ID NO: 78 - protein, 16 aa, synthetic construct
  SITE 1: note = N-terminus of Gly is protected with a Trt (trityl) protecting group
  SITE 5: note = Ser is protected as a dimethylated pseudoproline (Psi(Me,Me)pro) moiety in which the oxazolidine is derived from Ser
  SITE 10: note = Ser is protected as a dimethylated pseudoproline (Psi(Me,Me)pro) moiety in which the oxazolidine is derived from Ser
  SITE 15: note = Ser is protected as a dimethylated pseudoproline (Psi(Me,Me)pro) moiety in which the oxazolidine is derived from Ser
  SITE 16: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
GGGGSGGGGSGGGGSK

SEQ ID NO: 79 - protein, 24 aa, synthetic construct
  SITE 1: note = N-terminus of Ala is protected with a Fmoc (9-fluorenylmethyloxycarbonyl) protecting group
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Side chain of Ser is protected with a tert-butyl (tBu) protecting group
  SITE 18: note = Side chain of Ser is protected with a tert-butyl (tBu) protecting group
  SITE 23: note = Side chain of Ser is protected with a tert-butyl (tBu) protecting group
  SITE 24: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
AWLVKGGGGGGGSGGGGSGGGGSK

SEQ ID NO: 80 - protein, 24 aa, synthetic construct
  SITE 1: note = N-terminus of Ala is protected with a Fmoc (9-fluorenylmethyloxycarbonyl) protecting group
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Ser is protected as a dimethylated pseudoproline (Psi(Me,Me)pro) moiety in which the oxazolidine is derived from Ser
  SITE 18: note = Ser is protected as a dimethylated pseudoproline (Psi(Me,Me)pro) moiety in which the oxazolidine is derived from Ser
  SITE 23: note = Ser is protected as a dimethylated pseudoproline (Psi(Me,Me)pro) moiety in which the oxazolidine is derived from Ser
  SITE 24: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
AWLVKGGGGGGGSGGGGSGGGGSK

SEQ ID NO: 81 - protein, 34 aa, synthetic construct; identical to SEQ ID NO: 10
  SITE 1: note = N-terminus of Leu is protected with a Fmoc (9-fluorenylmethyloxycarbonyl) protecting group
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
  SITE 12: note = Side chain of Trp is protected with a Boc (tert-butyloxycarbonyl) protecting group
  SITE 15: note = Side chain of Lys is protected with a Boc (tert-butyloxycarbonyl) protecting group
  SITE 23: note = Side chain of Ser is protected with a tert-butyl (tBu) protecting group
  SITE 28: note = Side chain of Ser is protected with a tert-butyl (tBu) protecting group
  SITE 33: note = Side chain of Ser is protected with a tert-butyl (tBu) protecting group
  SITE 34: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group

SEQ ID NO: 82 - protein, 23 aa, synthetic construct
  SITE 1: note = N-terminus of Trp is protected with a Fmoc (9-fluorenylmethyloxycarbonyl) protecting group; Side chain of Trp is protected with a Boc (tert-butyloxycarbonyl) protecting group
  SITE 4: note = Side chain of Lys is protected with a Boc (tert-butyloxycarbonyl) protecting group
  SITE 12: note = Side chain of Ser is protected with a tert-butyl (tBu) protecting group
  SITE 17: note = Side chain of Ser is protected with a tert-butyl (tBu) protecting group
  SITE 22: note = Side chain of Ser is protected with a tert-butyl (tBu) protecting group
  SITE 23: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
WLVKGGGGGGGSGGGGSGGGGSK

SEQ ID NO: 83 - protein, 35 aa, synthetic construct; identical to SEQ ID NO: 14
  SITE 1: note = N-terminus of Tyr is protected with a Fmoc (9-fluorenylmethyloxycarbonyl) protecting group; Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
  SITE 13: note = Side chain of Trp is protected with a Boc (tert-butyloxycarbonyl) protecting group
  SITE 16: note = Side chain of Lys is protected with a Boc (tert-butyloxycarbonyl) protecting group
  SITE 24: note = Side chain of Ser is protected with a tert-butyl (tBu) protecting group
  SITE 29: note = Side chain of Ser is protected with a tert-butyl (tBu) protecting group
  SITE 34: note = Side chain of Ser is protected with a tert-butyl (tBu) protecting group
  SITE 35: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group

SEQ ID NO: 84 - protein, 12 aa, synthetic construct
  SITE 1: note = N-terminus of His is protected with a Boc (tert-butyloxycarbonyl) protecting group; Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OMpe (O-3-methylpent-3-yl) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
HXEGTFTSDYSS

SEQ ID NO: 85 - protein, 35 aa, synthetic construct; identical to SEQ ID NO: 14
  SITE 1: note = Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
  SITE 13: note = Side chain of Trp is protected with a Boc (tert-butyloxycarbonyl) protecting group
  SITE 16: note = Side chain of Lys is protected with a Boc (tert-butyloxycarbonyl) protecting group
  SITE 24: note = Side chain of Ser is protected with a tert-butyl (tBu) protecting group
  SITE 29: note = Side chain of Ser is protected with a tert-butyl (tBu) protecting group
  SITE 34: note = Side chain of Ser is protected with a tert-butyl (tBu) protecting group
  SITE 35: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide

SEQ ID NO: 86 - protein, 35 aa, synthetic construct; identical to SEQ ID NO: 14
  SITE 1: note = N-terminus of Tyr is protected; Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
  SITE 13: note = Side chain of Trp is protected with a Boc (tert-butyloxycarbonyl) protecting group
  SITE 16: note = Side chain of Lys is protected with a Boc (tert-butyloxycarbonyl) protecting group
  SITE 24: note = Side chain of Ser is protected with a tert-butyl (tBu) protecting group
  SITE 29: note = Side chain of Ser is protected with a tert-butyl (tBu) protecting group
  SITE 34: note = Side chain of Ser is protected with a tert-butyl (tBu) protecting group
  SITE 35: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide

SEQ ID NO: 87 - protein, 35 aa, synthetic construct; identical to SEQ ID NO: 14
  SITE 1: note = N-terminus of Tyr is protected with a Fmoc (9-fluorenylmethyloxycarbonyl) protecting group; Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
  SITE 13: note = Side chain of Trp is protected with a Boc (tert-butyloxycarbonyl) protecting group
  SITE 16: note = Side chain of Lys is protected with a Boc (tert-butyloxycarbonyl) protecting group
  SITE 24: note = Side chain of Ser is protected with a tert-butyl (tBu) protecting group
  SITE 29: note = Side chain of Ser is protected with a tert-butyl (tBu) protecting group
  SITE 34: note = Side chain of Ser is protected with a tert-butyl (tBu) protecting group
  SITE 35: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide

SEQ ID NO: 88 - protein, 35 aa, synthetic construct; identical to SEQ ID NO: 14
  SITE 1: note = Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
  SITE 13: note = Side chain of Trp is protected with a Boc (tert-butyloxycarbonyl) protecting group
  SITE 16: note = Side chain of Lys is protected with a Boc (tert-butyloxycarbonyl) protecting group
  SITE 24: note = Side chain of Ser is protected with a tert-butyl (tBu) protecting group
  SITE 29: note = Side chain of Ser is protected with a tert-butyl (tBu) protecting group
  SITE 34: note = Side chain of Ser is protected with a tert-butyl (tBu) protecting group
  SITE 35: note = Side chain of Lys is protected with an ivDde (1-(4,4-dimethyl-2,6-dioxocyclohex-1-ylidene)-3-methylbuty l) protecting group; C-terminus of Lys is protected

SEQ ID NO: 89 - protein, 35 aa, synthetic construct; identical to SEQ ID NO: 14
  SITE 1: note = N-terminus of Tyr is protected; Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
  SITE 13: note = Side chain of Trp is protected with a Boc (tert-butyloxycarbonyl) protecting group
  SITE 16: note = Side chain of Lys is protected with a Boc (tert-butyloxycarbonyl) protecting group
  SITE 24: note = Side chain of Ser is protected with a tert-butyl (tBu) protecting group
  SITE 29: note = Side chain of Ser is protected with a tert-butyl (tBu) protecting group
  SITE 34: note = Side chain of Ser is protected with a tert-butyl (tBu) protecting group
  SITE 35: note = Side chain of Lys is protected with an ivDde (1-(4,4-dimethyl-2,6-dioxocyclohex-1-ylidene)-3-methylbuty l) protecting group; C-terminus of Lys is protected

SEQ ID NO: 90 - protein, 35 aa, synthetic construct; identical to SEQ ID NO: 14
  SITE 1: note = N-terminus of Tyr is protected with a Fmoc (9-fluorenylmethyloxycarbonyl) protecting group; Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
  SITE 13: note = Side chain of Trp is protected with a Boc (tert-butyloxycarbonyl) protecting group
  SITE 16: note = Side chain of Lys is protected with a Boc (tert-butyloxycarbonyl) protecting group
  SITE 24: note = Side chain of Ser is protected with a tert-butyl (tBu) protecting group
  SITE 29: note = Side chain of Ser is protected with a tert-butyl (tBu) protecting group
  SITE 34: note = Side chain of Ser is protected with a tert-butyl (tBu) protecting group
  SITE 35: note = Side chain of Lys is protected with an ivDde (1-(4,4-dimethyl-2,6-dioxocyclohex-1-ylidene)-3-methylbuty l) protecting group; C-terminus of Lys is protected

SEQ ID NO: 91 - protein, 35 aa, synthetic construct; identical to SEQ ID NO: 14
  SITE 1: note = N-terminus of Tyr is protected with a Fmoc (9-fluorenylmethyloxycarbonyl) protecting group; Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
  SITE 13: note = Side chain of Trp is protected with a Boc (tert-butyloxycarbonyl) protecting group
  SITE 16: note = Side chain of Lys is protected with a Boc (tert-butyloxycarbonyl) protecting group
  SITE 24: note = Side chain of Ser is protected with a tert-butyl (tBu) protecting group
  SITE 29: note = Side chain of Ser is protected with a tert-butyl (tBu) protecting group
  SITE 34: note = Side chain of Ser is protected with a tert-butyl (tBu) protecting group
  SITE 35: note = Side chain of Lys is protected with an ivDde (1-(4,4-dimethyl-2,6-dioxocyclohex-1-ylidene)-3-methylbuty l) protecting group; C-terminus of Lys is amidated and is bound to a solid support through the C-terminal amide group

SEQ ID NO: 92 - protein, 12 aa, synthetic construct
  SITE 1: note = N-terminus of His is protected with a Fmoc (9-fluorenylmethyloxycarbonyl) protecting group; Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OMpe (O-3-methylpent-3-yl) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
HXEGTFTSDYSS

SEQ ID NO: 93 - protein, 12 aa, synthetic construct
  SITE 1: note = N-terminus of His is protected with a Boc (tert-butyloxycarbonyl) protecting group; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OMpe (O-3-methylpent-3-yl) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
HXEGTFTSDYSS

SEQ ID NO: 94 - protein, 12 aa, synthetic construct
  SITE 1: note = N-terminus of His is protected with a Fmoc (9-fluorenylmethyloxycarbonyl) protecting group; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OMpe (O-3-methylpent-3-yl) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
HXEGTFTSDYSS

SEQ ID NO: 95 - protein, 13 aa, synthetic construct
  SITE 1: note = N-terminus of His is protected with a Boc (tert-butyloxycarbonyl) protecting group; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Side chain of Ser is protected with a tert-butyl (tBu) protecting group
  SITE 13: note = Side chain of Tyr is protected with a tert-butyl (tBu) protecting group
HXEGTFTSDYSSY

SEQ ID NO: 96 - protein, 34 aa, synthetic construct; identical to SEQ ID NO: 10
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
  SITE 12: note = Side chain of Trp is protected with a Boc (tert-butyloxycarbonyl) protecting group
  SITE 15: note = Side chain of Lys is protected with a Boc (tert-butyloxycarbonyl) protecting group
  SITE 23: note = Side chain of Ser is protected with a tert-butyl (tBu) protecting group
  SITE 28: note = Side chain of Ser is protected with a tert-butyl (tBu) protecting group
  SITE 33: note = Side chain of Ser is protected with a tert-butyl (tBu) protecting group
  SITE 34: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide

SEQ ID NO: 97 - protein, 34 aa, synthetic construct; identical to SEQ ID NO: 10
  SITE 1: note = N-terminus of Leu is protected
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
  SITE 12: note = Side chain of Trp is protected with a Boc (tert-butyloxycarbonyl) protecting group
  SITE 15: note = Side chain of Lys is protected with a Boc (tert-butyloxycarbonyl) protecting group
  SITE 23: note = Side chain of Ser is protected with a tert-butyl (tBu) protecting group
  SITE 28: note = Side chain of Ser is protected with a tert-butyl (tBu) protecting group
  SITE 33: note = Side chain of Ser is protected with a tert-butyl (tBu) protecting group
  SITE 34: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide

SEQ ID NO: 98 - protein, 34 aa, synthetic construct; identical to SEQ ID NO: 10
  SITE 1: note = N-terminus of Leu is protected with a Fmoc (9-fluorenylmethyloxycarbonyl) protecting group
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
  SITE 12: note = Side chain of Trp is protected with a Boc (tert-butyloxycarbonyl) protecting group
  SITE 15: note = Side chain of Lys is protected with a Boc (tert-butyloxycarbonyl) protecting group
  SITE 23: note = Side chain of Ser is protected with a tert-butyl (tBu) protecting group
  SITE 28: note = Side chain of Ser is protected with a tert-butyl (tBu) protecting group
  SITE 33: note = Side chain of Ser is protected with a tert-butyl (tBu) protecting group
  SITE 34: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide

SEQ ID NO: 99 - protein, 13 aa, synthetic construct
  SITE 1: note = N-terminus of His is protected with a Fmoc (9-fluorenylmethyloxycarbonyl) protecting group; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Side chain of Ser is protected with a tert-butyl (tBu) protecting group
  SITE 13: note = Side chain of Tyr is protected with a tert-butyl (tBu) protecting group
HXEGTFTSDYSSY

SEQ ID NO: 100 - protein, 13 aa, synthetic construct
  SITE 1: note = N-terminus of His is protected with a Boc (tert-butyloxycarbonyl) protecting group; Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Side chain of Ser is protected with a tert-butyl (tBu) protecting group
  SITE 13: note = Side chain of Tyr is protected with a tert-butyl (tBu) protecting group
HXEGTFTSDYSSY

SEQ ID NO: 101 - protein, 13 aa, synthetic construct
  SITE 1: note = N-terminus of His is protected with a Fmoc (9-fluorenylmethyloxycarbonyl) protecting group; Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Side chain of Ser is protected with a tert-butyl (tBu) protecting group
  SITE 13: note = Side chain of Tyr is protected with a tert-butyl (tBu) protecting group
HXEGTFTSDYSSY

SEQ ID NO: 102 - protein, 12 aa, synthetic construct
  SITE 1: note = N-terminus of Tyr is protected; Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
  SITE 12: note = C-terminus of Ala is protected
YLEEQAAKEFIA

SEQ ID NO: 103 - protein, 10 aa, synthetic construct
  SITE 1: note = N-terminus of Leu is protected
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
  SITE 10: note = C-terminus of Ile is protected
LEEQAAKEFI

SEQ ID NO: 104 - protein, 11 aa, synthetic construct
  SITE 1: note = N-terminus of Tyr is protected; Side chain of Tyr is protected with a tert-butyl (tBu) protecting group
  SITE 3: note = Side chain of Glu is protected with a OtBu (tert-butyl ester) protecting group
  SITE 4: note = Side chain of Glu is protected with a OtBu (tert-butyl ester) protecting group
  SITE 5: note = Side chain of Gln is protected with a Trt (trityl) protecting group
  SITE 8: note = Side chain of Lys is protected with a Boc (tert-butyloxycarbonyl) protecting group
  SITE 9: note = Side chain of Glu is protected with a OtBu (tert-butyl ester) protecting group
  SITE 11: note = C-terminus of Ile is protected
YLEEQAAKEFI

SEQ ID NO: 105 - protein, 11 aa, synthetic construct
  SITE 1: note = N-terminus of Leu is protected
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
  SITE 11: note = C-terminus of Ala is protected
LEEQAAKEFIA

SEQ ID NO: 106 - protein, 11 aa, synthetic construct
  SITE 1: note = N-terminus of Trp is protected; Side chain of Trp is protected with a Boc (tert-butyloxycarbonyl) protecting group
  SITE 4: note = Side chain of Lys is protected with a Boc (tert-butyloxycarbonyl) protecting group
  SITE 11: note = C-terminus of Gly is protected
WLVKGGGGGGG

SEQ ID NO: 107 - protein, 12 aa, synthetic construct
  SITE 1: note = N-terminus of Ala is protected
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 12: note = C-terminus of Gly is protected
AWLVKGGGGGGG

SEQ ID NO: 108 - protein, 12 aa, synthetic construct
  SITE 1: note = Side chain of Ser is protected with a tert-butyl (tBu) protecting group
  SITE 6: note = Side chain of Ser is protected with a tert-butyl (tBu) protecting group
  SITE 11: note = Side chain of Ser is protected with a tert-butyl (tBu) protecting group
  SITE 12: note = Side chain of Lys is protected with an ivDde (1-(4,4-dimethyl-2,6-dioxocyclohex-1-ylidene)-3-methylbuty l) protecting group; C-terminus of Lys is protected
SGGGGSGGGGSK

SEQ ID NO: 109 - protein, 12 aa, synthetic construct
  SITE 1: note = Side chain of Ser is protected with a tert-butyl (tBu) protecting group
  SITE 6: note = Side chain of Ser is protected with a tert-butyl (tBu) protecting group
  SITE 11: note = Side chain of Ser is protected with a tert-butyl (tBu) protecting group
  SITE 12: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
SGGGGSGGGGSK

SEQ ID NO: 110 - protein, 12 aa, synthetic construct
  SITE 1: note = N-terminus of Ser is protected; Side chain of Ser is protected with a tert-butyl (tBu) protecting group
  SITE 6: note = Side chain of Ser is protected with a tert-butyl (tBu) protecting group
  SITE 11: note = Side chain of Ser is protected with a tert-butyl (tBu) protecting group
  SITE 12: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
SGGGGSGGGGSK

SEQ ID NO: 111 - protein, 12 aa, synthetic construct
  SITE 1: note = N-terminus of Ser is protected with a Fmoc (9-fluorenylmethyloxycarbonyl) protecting group; Side chain of Ser is protected with a tert-butyl (tBu) protecting group
  SITE 6: note = Side chain of Ser is protected with a tert-butyl (tBu) protecting group
  SITE 11: note = Side chain of Ser is protected with a tert-butyl (tBu) protecting group
  SITE 12: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
SGGGGSGGGGSK

SEQ ID NO: 112 - protein, 12 aa, synthetic construct
  SITE 1: note = N-terminus of Ser is protected with a Trt (trityl) protecting group; Side chain of Ser is protected with a tert-butyl (tBu) protecting group
  SITE 6: note = Side chain of Ser is protected with a tert-butyl (tBu) protecting group
  SITE 11: note = Side chain of Ser is protected with a tert-butyl (tBu) protecting group
  SITE 12: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
SGGGGSGGGGSK

SEQ ID NO: 113 - protein, 12 aa, synthetic construct
  SITE 1: note = N-terminus of Ser is protected with a Trt (trityl) protecting group; Side chain of Ser is protected with a tert-butyl (tBu) protecting group
  SITE 6: note = Side chain of Ser is protected with a tert-butyl (tBu) protecting group
  SITE 11: note = Side chain of Ser is protected with a tert-butyl (tBu) protecting group
  SITE 12: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
SGGGGSGGGGSK

SEQ ID NO: 114 - protein, 12 aa, synthetic construct
  SITE 1: note = N-terminus of Ser is protected; Side chain of Ser is protected with a tert-butyl (tBu) protecting group
  SITE 6: note = Side chain of Ser is protected with a tert-butyl (tBu) protecting group
  SITE 11: note = Side chain of Ser is protected with a tert-butyl (tBu) protecting group
  SITE 12: note = Side chain of Lys is protected with an ivDde (1-(4,4-dimethyl-2,6-dioxocyclohex-1-ylidene)-3-methylbuty l) protecting group; C-terminus of Ser is protected
SGGGGSGGGGSK

SEQ ID NO: 115 - protein, 12 aa, synthetic construct
  SITE 1: note = N-terminus of Ser is protected with a Fmoc (9-fluorenylmethyloxycarbonyl) protecting group; Side chain of Ser is protected with a tert-butyl (tBu) protecting group
  SITE 6: note = Side chain of Ser is protected with a tert-butyl (tBu) protecting group
  SITE 11: note = Side chain of Ser is protected with a tert-butyl (tBu) protecting group
  SITE 12: note = Side chain of Lys is protected with an ivDde (1-(4,4-dimethyl-2,6-dioxocyclohex-1-ylidene)-3-methylbuty l) protecting group; C-terminus of Lys is protected
SGGGGSGGGGSK

SEQ ID NO: 116 - protein, 12 aa, synthetic construct
  SITE 1: note = N-terminus of Ser is protected with a Trt (trityl) protecting group; Side chain of Ser is protected with a tert-butyl (tBu) protecting group
  SITE 6: note = Side chain of Ser is protected with a tert-butyl (tBu) protecting group
  SITE 11: note = Side chain of Ser is protected with a tert-butyl (tBu) protecting group
  SITE 12: note = Side chain of Lys is protected with an ivDde (1-(4,4-dimethyl-2,6-dioxocyclohex-1-ylidene)-3-methylbuty l) protecting group; C-terminus of Lys is protected
SGGGGSGGGGSK

SEQ ID NO: 117 - protein, 12 aa, synthetic construct
  SITE 1: note = N-terminus of Ser is protected with a Fmoc (9-fluorenylmethyloxycarbonyl) protecting group; Side chain of Ser is protected with a tert-butyl (tBu) protecting group
  SITE 6: note = Side chain of Ser is protected with a tert-butyl (tBu) protecting group
  SITE 11: note = Side chain of Ser is protected with a tert-butyl (tBu) protecting group
  SITE 12: note = Side chain of Lys is protected with an ivDde (1-(4,4-dimethyl-2,6-dioxocyclohex-1-ylidene)-3-methylbuty l) protecting group; C-terminus of Lys is amidated and is bound to a solid support through the C-terminal amide group
SGGGGSGGGGSK

SEQ ID NO: 118 - protein, 8 aa, synthetic construct
  SITE 1: note = N-terminus of Ala is protected
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 8: note = C-terminus of Gly is protected
AWLVKGGG

SEQ ID NO: 119 - protein, 8 aa, synthetic construct
  SITE 1: note = N-terminus of Ala is protected
  SITE 2: note = Side chain of Trp is protected with a Boc (tert-butyloxycarbonyl) protecting group
  SITE 8: note = C-terminus of Gly is protected
AWLVKGGG

SEQ ID NO: 120 - protein, 16 aa, synthetic construct
  SITE 5: note = Side chain of Ser is protected with a tert-butyl (tBu) protecting group
  SITE 10: note = Side chain of Ser is protected with a tert-butyl (tBu) protecting group
  SITE 15: note = Side chain of Ser is protected with a tert-butyl (tBu) protecting group
  SITE 16: note = Side chain of Lys is protected with an ivDde (1-(4,4-dimethyl-2,6-dioxocyclohex-1-ylidene)-3-methylbuty l) protecting group; C-terminus of Lys is protected
GGGGSGGGGSGGGGSK

SEQ ID NO: 121 - protein, 16 aa, synthetic construct
  SITE 5: note = Side chain of Ser is protected with a tert-butyl (tBu) protecting group
  SITE 10: note = Side chain of Ser is protected with a tert-butyl (tBu) protecting group
  SITE 15: note = Side chain of Ser is protected with a tert-butyl (tBu) protecting group
  SITE 16: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
GGGGSGGGGSGGGGSK

SEQ ID NO: 122 - protein, 16 aa, synthetic construct
  SITE 1: note = N-terminus of Gly is protected
  SITE 5: note = Side chain of Ser is protected with a tert-butyl (tBu) protecting group
  SITE 10: note = Side chain of Ser is protected with a tert-butyl (tBu) protecting group
  SITE 15: note = Side chain of Ser is protected with a tert-butyl (tBu) protecting group
  SITE 16: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
GGGGSGGGGSGGGGSK

SEQ ID NO: 123 - protein, 16 aa, synthetic construct
  SITE 1: note = N-terminus of Gly is protected with a Trt (trityl) protecting group
  SITE 5: note = Side chain of Ser is protected with a tert-butyl (tBu) protecting group
  SITE 10: note = Side chain of Ser is protected with a tert-butyl (tBu) protecting group
  SITE 15: note = Side chain of Ser is protected with a tert-butyl (tBu) protecting group
  SITE 16: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
GGGGSGGGGSGGGGSK

SEQ ID NO: 124 - protein, 16 aa, synthetic construct
  SITE 1: note = N-terminus of Gly is protected
  SITE 5: note = Side chain of Ser is protected with a tert-butyl (tBu) protecting group
  SITE 10: note = Side chain of Ser is protected with a tert-butyl (tBu) protecting group
  SITE 15: note = Side chain of Ser is protected with a tert-butyl (tBu) protecting group
  SITE 16: note = Side chain of Lys is protected with an ivDde (1-(4,4-dimethyl-2,6-dioxocyclohex-1-ylidene)-3-methylbuty l) protecting group; C-terminus of Lys is protected
GGGGSGGGGSGGGGSK

SEQ ID NO: 125 - protein, 16 aa, synthetic construct
  SITE 1: note = N-terminus of Gly is protected with a Fmoc (9-fluorenylmethyloxycarbonyl) protecting group
  SITE 5: note = Side chain of Ser is protected with a tert-butyl (tBu) protecting group
  SITE 10: note = Side chain of Ser is protected with a tert-butyl (tBu) protecting group
  SITE 15: note = Side chain of Ser is protected with a tert-butyl (tBu) protecting group
  SITE 16: note = Side chain of Lys is protected with an ivDde (1-(4,4-dimethyl-2,6-dioxocyclohex-1-ylidene)-3-methylbuty l) protecting group; C-terminus of Lys is protected
GGGGSGGGGSGGGGSK

SEQ ID NO: 126 - protein, 16 aa, synthetic construct
  SITE 1: note = N-terminus of Gly is protected with a Trt (trityl) protecting group
  SITE 5: note = Side chain of Ser is protected with a tert-butyl (tBu) protecting group
  SITE 10: note = Side chain of Ser is protected with a tert-butyl (tBu) protecting group
  SITE 15: note = Side chain of Ser is protected with a tert-butyl (tBu) protecting group
  SITE 16: note = Side chain of Lys is protected with an ivDde (1-(4,4-dimethyl-2,6-dioxocyclohex-1-ylidene)-3-methylbuty l) protecting group; C-terminus of Lys is protected
GGGGSGGGGSGGGGSK

SEQ ID NO: 127 - protein, 16 aa, synthetic construct
  SITE 1: note = N-terminus of Gly is protected with a Fmoc (9-fluorenylmethyloxycarbonyl) protecting group
  SITE 5: note = Side chain of Ser is protected with a tert-butyl (tBu) protecting group
  SITE 10: note = Side chain of Ser is protected with a tert-butyl (tBu) protecting group
  SITE 15: note = Side chain of Ser is protected with a tert-butyl (tBu) protecting group
  SITE 16: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
GGGGSGGGGSGGGGSK

SEQ ID NO: 128 - protein, 16 aa, synthetic construct
  SITE 1: note = N-terminus of Gly is protected with a Trt (trityl) protecting group
  SITE 5: note = Side chain of Ser is protected with a tert-butyl (tBu) protecting group
  SITE 10: note = Side chain of Ser is protected with a tert-butyl (tBu) protecting group
  SITE 15: note = Side chain of Ser is protected with a tert-butyl (tBu) protecting group
  SITE 16: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
GGGGSGGGGSGGGGSK

SEQ ID NO: 129 - protein, 16 aa, synthetic construct
  SITE 5: note = Ser is protected as a dimethylated pseudoproline (Psi(Me,Me)pro) moiety in which the oxazolidine is derived from Ser
  SITE 10: note = Ser is protected as a dimethylated pseudoproline (Psi(Me,Me)pro) moiety in which the oxazolidine is derived from Ser
  SITE 15: note = Ser is protected as a dimethylated pseudoproline (Psi(Me,Me)pro) moiety in which the oxazolidine is derived from Ser
  SITE 16: note = Side chain of Lys is protected with an ivDde (1-(4,4-dimethyl-2,6-dioxocyclohex-1-ylidene)-3-methylbuty l) protecting group; C-terminus of Lys is protected
GGGGSGGGGSGGGGSK

SEQ ID NO: 130 - protein, 16 aa, synthetic construct
  SITE 5: note = Ser is protected as a dimethylated pseudoproline (Psi(Me,Me)pro) moiety in which the oxazolidine is derived from Ser
  SITE 10: note = Ser is protected as a dimethylated pseudoproline (Psi(Me,Me)pro) moiety in which the oxazolidine is derived from Ser
  SITE 15: note = Ser is protected as a dimethylated pseudoproline (Psi(Me,Me)pro) moiety in which the oxazolidine is derived from Ser
  SITE 16: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
GGGGSGGGGSGGGGSK

SEQ ID NO: 131 - protein, 16 aa, synthetic construct
  SITE 1: note = N-terminus of Gly is protected
  SITE 5: note = Ser is protected as a dimethylated pseudoproline (Psi(Me,Me)pro) moiety in which the oxazolidine is derived from Ser
  SITE 10: note = Ser is protected as a dimethylated pseudoproline (Psi(Me,Me)pro) moiety in which the oxazolidine is derived from Ser
  SITE 15: note = Ser is protected as a dimethylated pseudoproline (Psi(Me,Me)pro) moiety in which the oxazolidine is derived from Ser
  SITE 16: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
GGGGSGGGGSGGGGSK

SEQ ID NO: 132 - protein, 16 aa, synthetic construct
  SITE 1: note = N-terminus of Gly is protected with a Fmoc (9-fluorenylmethyloxycarbonyl) protecting group
  SITE 5: note = Ser is protected as a dimethylated pseudoproline (Psi(Me,Me)pro) moiety in which the oxazolidine is derived from Ser
  SITE 10: note = Ser is protected as a dimethylated pseudoproline (Psi(Me,Me)pro) moiety in which the oxazolidine is derived from Ser
  SITE 15: note = Ser is protected as a dimethylated pseudoproline (Psi(Me,Me)pro) moiety in which the oxazolidine is derived from Ser
  SITE 16: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
GGGGSGGGGSGGGGSK

SEQ ID NO: 133 - protein, 16 aa, synthetic construct
  SITE 1: note = N-terminus of Gly is protected
  SITE 5: note = Ser is protected as a dimethylated pseudoproline (Psi(Me,Me)pro) moiety in which the oxazolidine is derived from Ser
  SITE 10: note = Ser is protected as a dimethylated pseudoproline (Psi(Me,Me)pro) moiety in which the oxazolidine is derived from Ser
  SITE 15: note = Ser is protected as a dimethylated pseudoproline (Psi(Me,Me)pro) moiety in which the oxazolidine is derived from Ser
  SITE 16: note = Side chain of Lys is protected with an ivDde (1-(4,4-dimethyl-2,6-dioxocyclohex-1-ylidene)-3-methylbuty l) protecting group; C-terminus of Lys is protected
GGGGSGGGGSGGGGSK

SEQ ID NO: 134 - protein, 16 aa, synthetic construct
  SITE 1: note = N-terminus of Gly is protected with a Fmoc (9-fluorenylmethyloxycarbonyl) protecting group
  SITE 5: note = Ser is protected as a dimethylated pseudoproline (Psi(Me,Me)pro) moiety in which the oxazolidine is derived from Ser
  SITE 10: note = Ser is protected as a dimethylated pseudoproline (Psi(Me,Me)pro) moiety in which the oxazolidine is derived from Ser
  SITE 15: note = Ser is protected as a dimethylated pseudoproline (Psi(Me,Me)pro) moiety in which the oxazolidine is derived from Ser
  SITE 16: note = Side chain of Lys is protected with an ivDde (1-(4,4-dimethyl-2,6-dioxocyclohex-1-ylidene)-3-methylbuty l) protecting group; C-terminus of Lys is protected
GGGGSGGGGSGGGGSK

SEQ ID NO: 135 - protein, 16 aa, synthetic construct
  SITE 1: note = N-terminus of Gly is protected with a Trt (trityl) protecting group
  SITE 5: note = Ser is protected as a dimethylated pseudoproline (Psi(Me,Me)pro) moiety in which the oxazolidine is derived from Ser
  SITE 10: note = Ser is protected as a dimethylated pseudoproline (Psi(Me,Me)pro) moiety in which the oxazolidine is derived from Ser
  SITE 15: note = Ser is protected as a dimethylated pseudoproline (Psi(Me,Me)pro) moiety in which the oxazolidine is derived from Ser
  SITE 16: note = Side chain of Lys is protected with an ivDde (1-(4,4-dimethyl-2,6-dioxocyclohex-1-ylidene)-3-methylbuty l) protecting group; C-terminus of Lys is protected
GGGGSGGGGSGGGGSK

SEQ ID NO: 136 - protein, 16 aa, synthetic construct
  SITE 1: note = N-terminus of Gly is protected with a Fmoc (9-fluorenylmethyloxycarbonyl) protecting group
  SITE 5: note = Ser is protected as a dimethylated pseudoproline (Psi(Me,Me)pro) moiety in which the oxazolidine is derived from Ser
  SITE 10: note = Ser is protected as a dimethylated pseudoproline (Psi(Me,Me)pro) moiety in which the oxazolidine is derived from Ser
  SITE 15: note = Ser is protected as a dimethylated pseudoproline (Psi(Me,Me)pro) moiety in which the oxazolidine is derived from Ser
  SITE 16: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
GGGGSGGGGSGGGGSK

SEQ ID NO: 137 - protein, 16 aa, synthetic construct
  SITE 1: note = N-terminus of Gly is protected with a Trt (trityl) protecting group
  SITE 5: note = Ser is protected as a dimethylated pseudoproline (Psi(Me,Me)pro) moiety in which the oxazolidine is derived from Ser
  SITE 10: note = Ser is protected as a dimethylated pseudoproline (Psi(Me,Me)pro) moiety in which the oxazolidine is derived from Ser
  SITE 15: note = Ser is protected as a dimethylated pseudoproline (Psi(Me,Me)pro) moiety in which the oxazolidine is derived from Ser
  SITE 16: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
GGGGSGGGGSGGGGSK

SEQ ID NO: 138 - protein, 16 aa, synthetic construct
  SITE 1: note = N-terminus of Gly is protected with a Fmoc (9-fluorenylmethyloxycarbonyl) protecting group
  SITE 5: note = Side chain of Ser is protected with a tert-butyl (tBu) protecting group
  SITE 10: note = Side chain of Ser is protected with a tert-butyl (tBu) protecting group
  SITE 15: note = Side chain of Ser is protected with a tert-butyl (tBu) protecting group
  SITE 16: note = Side chain of Lys is protected with an ivDde (1-(4,4-dimethyl-2,6-dioxocyclohex-1-ylidene)-3-methylbuty l) protecting group; C-terminus of Lys is amidated and is bound to a solid support through the C-terminal amide group
GGGGSGGGGSGGGGSK

SEQ ID NO: 139 - protein, 24 aa, synthetic construct
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Side chain of Ser is protected with a tert-butyl (tBu) protecting group
  SITE 18: note = Side chain of Ser is protected with a tert-butyl (tBu) protecting group
  SITE 23: note = Side chain of Ser is protected with a tert-butyl (tBu) protecting group
  SITE 24: note = Side chain of Lys is protected with an ivDde (1-(4,4-dimethyl-2,6-dioxocyclohex-1-ylidene)-3-methylbuty l) protecting group; C-terminus of Lys is protected
AWLVKGGGGGGGSGGGGSGGGGSK

SEQ ID NO: 140 - protein, 24 aa, synthetic construct
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Side chain of Ser is protected with a tert-butyl (tBu) protecting group
  SITE 18: note = Side chain of Ser is protected with a tert-butyl (tBu) protecting group
  SITE 23: note = Side chain of Ser is protected with a tert-butyl (tBu) protecting group
  SITE 24: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
AWLVKGGGGGGGSGGGGSGGGGSK

SEQ ID NO: 141 - protein, 24 aa, synthetic construct
  SITE 1: note = N-terminus of Ala is protected
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Side chain of Ser is protected with a tert-butyl (tBu) protecting group
  SITE 18: note = Side chain of Ser is protected with a tert-butyl (tBu) protecting group
  SITE 23: note = Side chain of Ser is protected with a tert-butyl (tBu) protecting group
  SITE 24: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
AWLVKGGGGGGGSGGGGSGGGGSK

SEQ ID NO: 142 - protein, 24 aa, synthetic construct
  SITE 1: note = N-terminus of Ala is protected
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Side chain of Ser is protected with a tert-butyl (tBu) protecting group
  SITE 18: note = Side chain of Ser is protected with a tert-butyl (tBu) protecting group
  SITE 23: note = Side chain of Ser is protected with a tert-butyl (tBu) protecting group
  SITE 24: note = Side chain of Lys is protected with an ivDde (1-(4,4-dimethyl-2,6-dioxocyclohex-1-ylidene)-3-methylbuty l) protecting group; C-terminus of Lys is protected
AWLVKGGGGGGGSGGGGSGGGGSK

SEQ ID NO: 143 - protein, 24 aa, synthetic construct
  SITE 1: note = N-terminus of Ala is protected with a Fmoc (9-fluorenylmethyloxycarbonyl) protecting group
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Side chain of Ser is protected with a tert-butyl (tBu) protecting group
  SITE 18: note = Side chain of Ser is protected with a tert-butyl (tBu) protecting group
  SITE 23: note = Side chain of Ser is protected with a tert-butyl (tBu) protecting group
  SITE 24: note = Side chain of Lys is protected with an ivDde (1-(4,4-dimethyl-2,6-dioxocyclohex-1-ylidene)-3-methylbuty l) protecting group; C-terminus of Lys is protected
AWLVKGGGGGGGSGGGGSGGGGSK

SEQ ID NO: 144 - protein, 24 aa, synthetic construct
  SITE 1: note = N-terminus of Ala is protected with a Fmoc (9-fluorenylmethyloxycarbonyl) protecting group
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Side chain of Ser is protected with a tert-butyl (tBu) protecting group
  SITE 18: note = Side chain of Ser is protected with a tert-butyl (tBu) protecting group
  SITE 23: note = Side chain of Ser is protected with a tert-butyl (tBu) protecting group
  SITE 24: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
AWLVKGGGGGGGSGGGGSGGGGSK

SEQ ID NO: 145 - protein, 24 aa, synthetic construct
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Ser is protected as a dimethylated pseudoproline (Psi(Me,Me)pro) moiety in which the oxazolidine is derived from Ser
  SITE 18: note = Ser is protected as a dimethylated pseudoproline (Psi(Me,Me)pro) moiety in which the oxazolidine is derived from Ser
  SITE 23: note = Ser is protected as a dimethylated pseudoproline (Psi(Me,Me)pro) moiety in which the oxazolidine is derived from Ser
  SITE 24: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
AWLVKGGGGGGGSGGGGSGGGGSK

SEQ ID NO: 146 - protein, 24 aa, synthetic construct
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Ser is protected as a dimethylated pseudoproline (Psi(Me,Me)pro) moiety in which the oxazolidine is derived from Ser
  SITE 18: note = Ser is protected as a dimethylated pseudoproline (Psi(Me,Me)pro) moiety in which the oxazolidine is derived from Ser
  SITE 23: note = Ser is protected as a dimethylated pseudoproline (Psi(Me,Me)pro) moiety in which the oxazolidine is derived from Ser
  SITE 24: note = Side chain of Lys is protected with an ivDde (1-(4,4-dimethyl-2,6-dioxocyclohex-1-ylidene)-3-methylbuty l) protecting group; C-terminus of Lys is protected
AWLVKGGGGGGGSGGGGSGGGGSK

SEQ ID NO: 147 - protein, 24 aa, synthetic construct
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Ser is protected as a dimethylated pseudoproline (Psi(Me,Me)pro) moiety in which the oxazolidine is derived from Ser
  SITE 18: note = Ser is protected as a dimethylated pseudoproline (Psi(Me,Me)pro) moiety in which the oxazolidine is derived from Ser
  SITE 23: note = Ser is protected as a dimethylated pseudoproline (Psi(Me,Me)pro) moiety in which the oxazolidine is derived from Ser
  SITE 24: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
AWLVKGGGGGGGSGGGGSGGGGSK

SEQ ID NO: 148 - protein, 24 aa, synthetic construct
  SITE 1: note = N-terminus of Ala is protected
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Ser is protected as a dimethylated pseudoproline (Psi(Me,Me)pro) moiety in which the oxazolidine is derived from Ser
  SITE 18: note = Ser is protected as a dimethylated pseudoproline (Psi(Me,Me)pro) moiety in which the oxazolidine is derived from Ser
  SITE 23: note = Ser is protected as a dimethylated pseudoproline (Psi(Me,Me)pro) moiety in which the oxazolidine is derived from Ser
  SITE 24: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
AWLVKGGGGGGGSGGGGSGGGGSK

SEQ ID NO: 149 - protein, 24 aa, synthetic construct
  SITE 1: note = N-terminus of Ala is protected
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Ser is protected as a dimethylated pseudoproline (Psi(Me,Me)pro) moiety in which the oxazolidine is derived from Ser
  SITE 18: note = Ser is protected as a dimethylated pseudoproline (Psi(Me,Me)pro) moiety in which the oxazolidine is derived from Ser
  SITE 23: note = Ser is protected as a dimethylated pseudoproline (Psi(Me,Me)pro) moiety in which the oxazolidine is derived from Ser
  SITE 24: note = Side chain of Lys is protected with an ivDde (1-(4,4-dimethyl-2,6-dioxocyclohex-1-ylidene)-3-methylbuty l) protecting group; C-terminus of Lys is protected
AWLVKGGGGGGGSGGGGSGGGGSK

SEQ ID NO: 150 - protein, 24 aa, synthetic construct
  SITE 1: note = N-terminus of Ala is protected with a Fmoc (9-fluorenylmethyloxycarbonyl) protecting group
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Ser is protected as a dimethylated pseudoproline (Psi(Me,Me)pro) moiety in which the oxazolidine is derived from Ser
  SITE 18: note = Ser is protected as a dimethylated pseudoproline (Psi(Me,Me)pro) moiety in which the oxazolidine is derived from Ser
  SITE 23: note = Ser is protected as a dimethylated pseudoproline (Psi(Me,Me)pro) moiety in which the oxazolidine is derived from Ser
  SITE 24: note = Side chain of Lys is protected with an ivDde (1-(4,4-dimethyl-2,6-dioxocyclohex-1-ylidene)-3-methylbuty l) protecting group; C-terminus of Lys is protected
AWLVKGGGGGGGSGGGGSGGGGSK

SEQ ID NO: 151 - protein, 24 aa, synthetic construct
  SITE 1: note = N-terminus of Ala is protected with a Fmoc (9-fluorenylmethyloxycarbonyl) protecting group
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Ser is protected as a dimethylated pseudoproline (Psi(Me,Me)pro) moiety in which the oxazolidine is derived from Ser
  SITE 18: note = Ser is protected as a dimethylated pseudoproline (Psi(Me,Me)pro) moiety in which the oxazolidine is derived from Ser
  SITE 23: note = Ser is protected as a dimethylated pseudoproline (Psi(Me,Me)pro) moiety in which the oxazolidine is derived from Ser
  SITE 24: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
AWLVKGGGGGGGSGGGGSGGGGSK

SEQ ID NO: 152 - protein, 24 aa, synthetic construct
  SITE 1: note = N-terminus of Ala is protected with a Fmoc (9-fluorenylmethyloxycarbonyl) protecting group
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Side chain of Ser is protected with a tert-butyl (tBu) protecting group
  SITE 18: note = Side chain of Ser is protected with a tert-butyl (tBu) protecting group
  SITE 23: note = Side chain of Ser is protected with a tert-butyl (tBu) protecting group
  SITE 24: note = Side chain of Lys is protected with an ivDde (1-(4,4-dimethyl-2,6-dioxocyclohex-1-ylidene)-3-methylbuty l) protecting group; C-terminus of Lys is amidated and is bound to a solid support through the C-terminal amide group
AWLVKGGGGGGGSGGGGSGGGGSK

SEQ ID NO: 153 - protein, 34 aa, synthetic construct; identical to SEQ ID NO: 10
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
  SITE 12: note = Side chain of Trp is protected with a Boc (tert-butyloxycarbonyl) protecting group
  SITE 15: note = Side chain of Lys is protected with a Boc (tert-butyloxycarbonyl) protecting group
  SITE 23: note = Side chain of Ser is protected with a tert-butyl (tBu) protecting group
  SITE 28: note = Side chain of Ser is protected with a tert-butyl (tBu) protecting group
  SITE 33: note = Side chain of Ser is protected with a tert-butyl (tBu) protecting group
  SITE 34: note = Side chain of Lys is protected with an ivDde (1-(4,4-dimethyl-2,6-dioxocyclohex-1-ylidene)-3-methylbuty l) protecting group; C-terminus of Lys is protected

SEQ ID NO: 154 - protein, 34 aa, synthetic construct; identical to SEQ ID NO: 10
  SITE 1: note = N-terminus of Leu is protected
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
  SITE 12: note = Side chain of Trp is protected with a Boc (tert-butyloxycarbonyl) protecting group
  SITE 15: note = Side chain of Lys is protected with a Boc (tert-butyloxycarbonyl) protecting group
  SITE 23: note = Side chain of Ser is protected with a tert-butyl (tBu) protecting group
  SITE 28: note = Side chain of Ser is protected with a tert-butyl (tBu) protecting group
  SITE 33: note = Side chain of Ser is protected with a tert-butyl (tBu) protecting group
  SITE 34: note = Side chain of Lys is protected with an ivDde (1-(4,4-dimethyl-2,6-dioxocyclohex-1-ylidene)-3-methylbuty l) protecting group; C-terminus of Lys is protected

SEQ ID NO: 155 - protein, 34 aa, synthetic construct; identical to SEQ ID NO: 10
  SITE 1: note = N-terminus of Leu is protected with a Fmoc (9-fluorenylmethyloxycarbonyl) protecting group
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
  SITE 12: note = Side chain of Trp is protected with a Boc (tert-butyloxycarbonyl) protecting group
  SITE 15: note = Side chain of Lys is protected with a Boc (tert-butyloxycarbonyl) protecting group
  SITE 23: note = Side chain of Ser is protected with a tert-butyl (tBu) protecting group
  SITE 28: note = Side chain of Ser is protected with a tert-butyl (tBu) protecting group
  SITE 33: note = Side chain of Ser is protected with a tert-butyl (tBu) protecting group
  SITE 34: note = Side chain of Lys is protected with an ivDde (1-(4,4-dimethyl-2,6-dioxocyclohex-1-ylidene)-3-methylbuty l) protecting group; C-terminus of Lys is protected

SEQ ID NO: 156 - protein, 34 aa, synthetic construct; identical to SEQ ID NO: 10
  SITE 1: note = N-terminus of Leu is protected with a Fmoc (9-fluorenylmethyloxycarbonyl) protecting group
  SITE 2: note = Side chain of Glu is protected with a OtBu (tert-butyl ester) protecting group
  SITE 3: note = Side chain of Glu is protected with a OtBu (tert-butyl ester) protecting group
  SITE 4: note = Side chain of Gln is protected with a Trt (trityl) protecting group
  SITE 7: note = Side chain of Lys is protected with a Boc (tert-butyloxycarbonyl) protecting group
  SITE 8: note = Side chain of Glu is protected with a OtBu (tert-butyl ester) protecting group
  SITE 12: note = Side chain of Trp is protected with a Boc (tert-butyloxycarbonyl) protecting group
  SITE 15: note = Side chain of Lys is protected with a Boc (tert-butyloxycarbonyl) protecting group
  SITE 23: note = Side chain of Ser is protected with a tert-butyl (tBu) protecting group
  SITE 28: note = Side chain of Ser is protected with a tert-butyl (tBu) protecting group
  SITE 33: note = Side chain of Ser is protected with a tert-butyl (tBu) protecting group
  SITE 34: note = Side chain of Lys is protected with an ivDde (1-(4,4-dimethyl-2,6-dioxocyclohex-1-ylidene)-3-methylbuty l) protecting group; C-terminus of Lys is amidated and is bound to a solid support through the C-terminal amide group

SEQ ID NO: 157 - protein, 23 aa, synthetic construct
  SITE 1: note = N-terminus of His is protected with a Boc (tert-butyloxycarbonyl) protecting group; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
HXEGTFTSDYSSYLEEQAAKEFI

SEQ ID NO: 158 - protein, 23 aa, synthetic construct
  SITE 1: note = N-terminus of His is protected with a Fmoc (9-fluorenylmethyloxycarbonyl) protecting group; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
HXEGTFTSDYSSYLEEQAAKEFI

SEQ ID NO: 159 - protein, 23 aa, synthetic construct
  SITE 1: note = Side chain of Trp is protected with a Boc (tert-butyloxycarbonyl) protecting group
  SITE 4: note = Side chain of Lys is protected with a Boc (tert-butyloxycarbonyl) protecting group
  SITE 12: note = Side chain of Ser is protected with a tert-butyl (tBu) protecting group
  SITE 17: note = Side chain of Ser is protected with a tert-butyl (tBu) protecting group
  SITE 22: note = Side chain of Ser is protected with a tert-butyl (tBu) protecting group
  SITE 23: note = Side chain of Lys is protected with an ivDde (1-(4,4-dimethyl-2,6-dioxocyclohex-1-ylidene)-3-methylbuty l) protecting group; C-terminus of Lys is protected
WLVKGGGGGGGSGGGGSGGGGSK

SEQ ID NO: 160 - protein, 23 aa, synthetic construct
  SITE 1: note = Side chain of Trp is protected with a Boc (tert-butyloxycarbonyl) protecting group
  SITE 4: note = Side chain of Lys is protected with a Boc (tert-butyloxycarbonyl) protecting group
  SITE 12: note = Side chain of Ser is protected with a tert-butyl (tBu) protecting group
  SITE 17: note = Side chain of Ser is protected with a tert-butyl (tBu) protecting group
  SITE 22: note = Side chain of Ser is protected with a tert-butyl (tBu) protecting group
  SITE 23: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
WLVKGGGGGGGSGGGGSGGGGSK

SEQ ID NO: 161 - protein, 23 aa, synthetic construct
  SITE 1: note = N-terminus of Trp is protected; Side chain of Trp is protected with a Boc (tert-butyloxycarbonyl) protecting group
  SITE 4: note = Side chain of Lys is protected with a Boc (tert-butyloxycarbonyl) protecting group
  SITE 12: note = Side chain of Ser is protected with a tert-butyl (tBu) protecting group
  SITE 17: note = Side chain of Ser is protected with a tert-butyl (tBu) protecting group
  SITE 22: note = Side chain of Ser is protected with a tert-butyl (tBu) protecting group
  SITE 23: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
WLVKGGGGGGGSGGGGSGGGGSK

SEQ ID NO: 162 - protein, 23 aa, synthetic construct
  SITE 1: note = N-terminus of Trp is protected; Side chain of Trp is protected with a Boc (tert-butyloxycarbonyl) protecting group
  SITE 4: note = Side chain of Lys is protected with a Boc (tert-butyloxycarbonyl) protecting group
  SITE 12: note = Side chain of Ser is protected with a tert-butyl (tBu) protecting group
  SITE 17: note = Side chain of Ser is protected with a tert-butyl (tBu) protecting group
  SITE 22: note = Side chain of Ser is protected with a tert-butyl (tBu) protecting group
  SITE 23: note = Side chain of Lys is protected with an ivDde (1-(4,4-dimethyl-2,6-dioxocyclohex-1-ylidene)-3-methylbuty l) protecting group; C-terminus of Lys is protected
WLVKGGGGGGGSGGGGSGGGGSK

SEQ ID NO: 163 - protein, 23 aa, synthetic construct
  SITE 1: note = N-terminus of Trp is protected with a Fmoc (9-fluorenylmethyloxycarbonyl) protecting group; Side chain of Trp is protected with a Boc (tert-butyloxycarbonyl) protecting group
  SITE 4: note = Side chain of Lys is protected with a Boc (tert-butyloxycarbonyl) protecting group
  SITE 12: note = Side chain of Ser is protected with a tert-butyl (tBu) protecting group
  SITE 17: note = Side chain of Ser is protected with a tert-butyl (tBu) protecting group
  SITE 22: note = Side chain of Ser is protected with a tert-butyl (tBu) protecting group
  SITE 23: note = Side chain of Lys is protected with an ivDde (1-(4,4-dimethyl-2,6-dioxocyclohex-1-ylidene)-3-methylbuty l) protecting group; C-terminus of Lys is protected
WLVKGGGGGGGSGGGGSGGGGSK

SEQ ID NO: 164 - protein, 23 aa, synthetic construct
  SITE 1: note = N-terminus of Trp is protected with a Fmoc (9-fluorenylmethyloxycarbonyl) protecting group; Side chain of Trp is protected with a Boc (tert-butyloxycarbonyl) protecting group
  SITE 4: note = Side chain of Lys is protected with a Boc (tert-butyloxycarbonyl) protecting group
  SITE 12: note = Side chain of Ser is protected with a tert-butyl (tBu) protecting group
  SITE 17: note = Side chain of Ser is protected with a tert-butyl (tBu) protecting group
  SITE 22: note = Side chain of Ser is protected with a tert-butyl (tBu) protecting group
  SITE 23: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
WLVKGGGGGGGSGGGGSGGGGSK

SEQ ID NO: 165 - protein, 23 aa, synthetic construct
  SITE 1: note = N-terminus of Trp is protected with a Fmoc (9-fluorenylmethyloxycarbonyl) protecting group; Side chain of Trp is protected with a Boc (tert-butyloxycarbonyl) protecting group
  SITE 4: note = Side chain of Lys is protected with a Boc (tert-butyloxycarbonyl) protecting group
  SITE 12: note = Side chain of Ser is protected with a tert-butyl (tBu) protecting group
  SITE 17: note = Side chain of Ser is protected with a tert-butyl (tBu) protecting group
  SITE 22: note = Side chain of Ser is protected with a tert-butyl (tBu) protecting group
  SITE 23: note = Side chain of Lys is protected with an ivDde (1-(4,4-dimethyl-2,6-dioxocyclohex-1-ylidene)-3-methylbuty l) protecting group; C-terminus of Lys is amidated and is bound to a solid support through the C-terminal amide group
WLVKGGGGGGGSGGGGSGGGGSK

SEQ ID NO: 166 - protein, 12 aa, synthetic construct
  SITE 1: note = N-terminus of His is protected with a Boc (tert-butyloxycarbonyl) protecting group; Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
HXEGTFTSDYSS

SEQ ID NO: 167 - protein, 12 aa, synthetic construct
  SITE 1: note = N-terminus of His is protected with a Fmoc (9-fluorenylmethyloxycarbonyl) protecting group; Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
HXEGTFTSDYSS

SEQ ID NO: 168 - protein, 23 aa, synthetic construct
  SITE 1: note = N-terminus of His is protected with a Boc (tert-butyloxycarbonyl) protecting group; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Side chain of Ser is protected with a tert-butyl (tBu) protecting group
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
HXEGTFTSDYSSYLEEQAAKEFI

SEQ ID NO: 169 - protein, 23 aa, synthetic construct
  SITE 1: note = N-terminus of His is protected with a Boc (tert-butyloxycarbonyl) protecting group; Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
HXEGTFTSDYSSYLEEQAAKEFI

SEQ ID NO: 170 - protein, 24 aa, synthetic construct
  SITE 1: note = N-terminus of Ala is protected with a Trt (trityl) protecting group
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Ser is protected as a dimethylated pseudoproline (Psi(Me,Me)pro) moiety in which the oxazolidine is derived from Ser
  SITE 18: note = Ser is protected as a dimethylated pseudoproline (Psi(Me,Me)pro) moiety in which the oxazolidine is derived from Ser
  SITE 23: note = Ser is protected as a dimethylated pseudoproline (Psi(Me,Me)pro) moiety in which the oxazolidine is derived from Ser
  SITE 24: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide
AWLVKGGGGGGGSGGGGSGGGGSK

SEQ ID NO: 171 - protein, 47 aa, synthetic construct; identical to SEQ ID NO: 7
  SITE 1: note = N-terminus of His is protected with a Boc (tert-butyloxycarbonyl) protecting group; Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
  SITE 25: note = Side chain of Trp is protected with a Boc (tert-butyloxycarbonyl) protecting group
  SITE 28: note = Side chain of Lys is protected with a Boc (tert-butyloxycarbonyl) protecting group
  SITE 36: note = Ser is protected as a dimethylated pseudoproline (Psi(Me,Me)pro) moiety in which the oxazolidine is derived from Ser
  SITE 41: note = Ser is protected as a dimethylated pseudoproline (Psi(Me,Me)pro) moiety in which the oxazolidine is derived from Ser
  SITE 46: note = Ser is protected as a dimethylated pseudoproline (Psi(Me,Me)pro) moiety in which the oxazolidine is derived from Ser
  SITE 47: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide

SEQ ID NO: 172 - protein, 24 aa, synthetic construct
  SITE 1: note = N-terminus of Ala is protected with a Trt (trityl) protecting group
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Ser is protected as a dimethylated pseudoproline (Psi(Me,Me)pro) moiety in which the oxazolidine is derived from Ser
  SITE 18: note = Ser is protected as a dimethylated pseudoproline (Psi(Me,Me)pro) moiety in which the oxazolidine is derived from Ser
  SITE 23: note = Ser is protected as a dimethylated pseudoproline (Psi(Me,Me)pro) moiety in which the oxazolidine is derived from Ser
  SITE 24: note = Side chain of Lys is protected with an ivDde (1-(4,4-dimethyl-2,6-dioxocyclohex-1-ylidene)-3-methylbuty l) protecting group; C-terminus of Lys is protected
AWLVKGGGGGGGSGGGGSGGGGSK

SEQ ID NO: 173 - protein, 47 aa, synthetic construct; identical to SEQ ID NO: 7
  SITE 1: note = N-terminus of His is protected with a Boc (tert-butyloxycarbonyl) protecting group; Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
  SITE 25: note = Side chain of Trp is protected with a Boc (tert-butyloxycarbonyl) protecting group
  SITE 28: note = Side chain of Lys is protected with a Boc (tert-butyloxycarbonyl) protecting group
  SITE 36: note = Ser is protected as a dimethylated pseudoproline (Psi(Me,Me)pro) moiety in which the oxazolidine is derived from Ser
  SITE 41: note = Ser is protected as a dimethylated pseudoproline (Psi(Me,Me)pro) moiety in which the oxazolidine is derived from Ser
  SITE 46: note = Ser is protected as a dimethylated pseudoproline (Psi(Me,Me)pro) moiety in which the oxazolidine is derived from Ser
  SITE 47: note = C-terminus of Lys is amidated as a C-terminal primary amide

SEQ ID NO: 174 - protein, 47 aa, synthetic construct; identical to SEQ ID NO: 7
  SITE 1: note = N-terminus of His is protected with a Boc (tert-butyloxycarbonyl) protecting group; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Side chain of Ser is protected with a tert-butyl (tBu) protecting group
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
  SITE 25: note = Side chain of Trp is protected with a Boc (tert-butyloxycarbonyl) protecting group
  SITE 28: note = Side chain of Lys is protected with a Boc (tert-butyloxycarbonyl) protecting group
  SITE 36: note = Side chain of Ser is protected with a tert-butyl (tBu) protecting group
  SITE 41: note = Side chain of Ser is protected with a tert-butyl (tBu) protecting group
  SITE 46: note = Side chain of Ser is protected with a tert-butyl (tBu) protecting group
  SITE 47: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide

SEQ ID NO: 175 - protein, 23 aa, synthetic construct
  SITE 1: note = Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
HXEGTFTSDYSSYLEEQAAKEFI

SEQ ID NO: 176 - protein, 23 aa, synthetic construct
  SITE 1: note = N-terminus of His is protected; Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
HXEGTFTSDYSSYLEEQAAKEFI

SEQ ID NO: 177 - protein, 23 aa, synthetic construct
  SITE 1: note = N-terminus of His is protected with a Fmoc (9-fluorenylmethyloxycarbonyl) protecting group; Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
HXEGTFTSDYSSYLEEQAAKEFI

SEQ ID NO: 178 - protein, 24 aa, synthetic construct
  SITE 1: note = N-terminus of Ala is protected with a Trt (trityl) protecting group
  SITE 2: note = Side chain of Trp is protected with a Boc (tert-butyloxycarbonyl) protecting group
  SITE 5: note = Side chain of Lys is protected with a Boc (tert-butyloxycarbonyl) protecting group
  SITE 13: note = Ser is protected as a dimethylated pseudoproline (Psi(Me,Me)pro) moiety in which the oxazolidine is derived from Ser
  SITE 18: note = Ser is protected as a dimethylated pseudoproline (Psi(Me,Me)pro) moiety in which the oxazolidine is derived from Ser
  SITE 23: note = Ser is protected as a dimethylated pseudoproline (Psi(Me,Me)pro) moiety in which the oxazolidine is derived from Ser
  SITE 24: note = Side chain of Lys is protected with an ivDde (1-(4,4-dimethyl-2,6-dioxocyclohex-1-ylidene)-3-methylbuty l) protecting group; C-terminus of Lys does not have a free C-terminal carboxyl group
AWLVKGGGGGGGSGGGGSGGGGSK

SEQ ID NO: 179 - protein, 47 aa, synthetic construct; identical to SEQ ID NO: 7
  SITE 1: note = N-terminus of His is protected with a Boc (tert-butyloxycarbonyl) protecting group; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Side chain of Ser is protected with a tert-butyl (tBu) protecting group
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
  SITE 25: note = Side chain of Trp is protected with a Boc (tert-butyloxycarbonyl) protecting group
  SITE 28: note = Side chain of Lys is protected with a Boc (tert-butyloxycarbonyl) protecting group
  SITE 36: note = Side chain of Ser is protected with a tert-butyl (tBu) protecting group
  SITE 41: note = Side chain of Ser is protected with a tert-butyl (tBu) protecting group
  SITE 46: note = Side chain of Ser is protected with a tert-butyl (tBu) protecting group
  SITE 47: note = Side chain of Lys is protected with an ivDde (1-(4,4-dimethyl-2,6-dioxocyclohex-1-ylidene)-3-methylbuty l) protecting group; C-terminus of Lys is amidated and is bound to a solid support through the C-terminal amide group

SEQ ID NO: 180 - protein, 23 aa, synthetic construct
  SITE 1: note = Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Side chain of Ser is protected with a tert-butyl (tBu) protecting group
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
HXEGTFTSDYSSYLEEQAAKEFI

SEQ ID NO: 181 - protein, 12 aa, synthetic construct
  SITE 1: note = N-terminus of His is protected with a Boc (tert-butyloxycarbonyl) protecting group; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
HXEGTFTSDYSS

SEQ ID NO: 182 - protein, 12 aa, synthetic construct
  SITE 1: note = N-terminus of His is protected with a Fmoc (9-fluorenylmethyloxycarbonyl) protecting group; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
HXEGTFTSDYSS

SEQ ID NO: 183 - protein, 12 aa, synthetic construct
  SITE 1: note = Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
HXEGTFTSDYSS

SEQ ID NO: 184 - protein, 12 aa, synthetic construct
  SITE 1: note = N-terminus of His is protected; Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
HXEGTFTSDYSS

SEQ ID NO: 185 - protein, 12 aa, synthetic construct
  SITE 1: note = Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
HXEGTFTSDYSS

SEQ ID NO: 186 - protein, 12 aa, synthetic construct
  SITE 1: note = N-terminus of His is protected; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
HXEGTFTSDYSS

SEQ ID NO: 187 - protein, 8 aa, synthetic construct
  SITE 1: note = N-terminus of Ala is protected with a Trt (trityl) protecting group
  SITE 2: note = Side chain of Trp is protected with a Boc (tert-butyloxycarbonyl) protecting group
AWLVKGGG

SEQ ID NO: 188 - protein, 15 aa, synthetic construct
  SITE 1: note = N-terminus of Gly is protected with a Fmoc (9-fluorenylmethyloxycarbonyl) protecting group
  SITE 4: note = Side chain of Ser is protected with a tert-butyl (tBu) protecting group
  SITE 9: note = Side chain of Ser is protected with a tert-butyl (tBu) protecting group
  SITE 14: note = Side chain of Ser is protected with a tert-butyl (tBu) protecting group
  SITE 15: note = Side chain of Lys is protected with an ivDde (1-(4,4-dimethyl-2,6-dioxocyclohex-1-ylidene)-3-methylbuty l) protecting group; C-terminus of Lys is amidated and is bound to a solid support through the C-terminal amide group
GGGSGGGGSGGGGSK

SEQ ID NO: 189 - protein, 47 aa, synthetic construct; identical to SEQ ID NO: 7
  SITE 1: note = N-terminus of His is protected with a Boc (tert-butyloxycarbonyl) protecting group; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Side chain of Ser is protected with a tert-butyl (tBu) protecting group
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
  SITE 25: note = Side chain of Trp is protected with a Boc (tert-butyloxycarbonyl) protecting group
  SITE 28: note = Side chain of Lys is protected with a Boc (tert-butyloxycarbonyl) protecting group
  SITE 36: note = Ser is protected as a dimethylated pseudoproline (Psi(Me,Me)pro) moiety in which the oxazolidine is derived from Ser
  SITE 41: note = Ser is protected as a dimethylated pseudoproline (Psi(Me,Me)pro) moiety in which the oxazolidine is derived from Ser
  SITE 46: note = Ser is protected as a dimethylated pseudoproline (Psi(Me,Me)pro) moiety in which the oxazolidine is derived from Ser
  SITE 47: note = Side chain of Lys is protected with an ivDde (1-(4,4-dimethyl-2,6-dioxocyclohex-1-ylidene)-3-methylbuty l) protecting group; C-terminus of Lys is amidated as a C-terminal primary amide

SEQ ID NO: 190 - protein, 47 aa, synthetic construct; identical to SEQ ID NO: 7
  SITE 1: note = N-terminus of His is protected with a Boc (tert-butyloxycarbonyl) protecting group; Side chain of His is protected with a Boc (tert-butyloxycarbonyl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Side chain of Ser is protected with a tert-butyl (tBu) protecting group
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
  SITE 25: note = Side chain of Trp is protected with a Boc (tert-butyloxycarbonyl) protecting group
  SITE 28: note = Side chain of Lys is protected with a Boc (tert-butyloxycarbonyl) protecting group
  SITE 36: note = Side chain of Ser is protected with a tert-butyl (tBu) protecting group
  SITE 41: note = Side chain of Ser is protected with a tert-butyl (tBu) protecting group
  SITE 46: note = Side chain of Ser is protected with a tert-butyl (tBu) protecting group
  SITE 47: note = C-terminus of Lys is amidated as a C-terminal primary amide

SEQ ID NO: 191 - protein, 47 aa, synthetic construct; identical to SEQ ID NO: 7
  SITE 1: note = N-terminus of His is protected with a Boc (tert-butyloxycarbonyl) protecting group; Side chain of His is protected with a Trt (trityl) protecting group
  SITE 2: note = Aib
  SITE 3: note = Side chain of Glu is protected with a OtBu (tert-butyl ester) protecting group
  SITE 5: note = Side chain of Thr is protected with a tert-butyl (tBu) protecting group
  SITE 7: note = Side chain of Thr is protected with a tert-butyl (tBu) protecting group
  SITE 8: note = Side chain of Ser is protected with a tert-butyl (tBu) protecting group
  SITE 9: note = Side chain of Asp is protected with a OtBu (tert-butyl ester) protecting group
  SITE 10: note = Side chain of Tyr is protected with a tert-butyl (tBu) protecting group
  SITE 11: note = Side chain of Ser is protected with a tert-butyl (tBu) protecting group
  SITE 12: note = Ser is protected as a dimethylated pseudoproline (Psi(Me,Me)pro) moiety in which the oxazolidine is derived from Ser
  SITE 13: note = Side chain of Tyr is protected with a tert-butyl (tBu) protecting group
  SITE 15: note = Side chain of Glu is protected with a OtBu (tert-butyl ester) protecting group
  SITE 16: note = Side chain of Glu is protected with a OtBu (tert-butyl ester) protecting group
  SITE 17: note = Side chain of Gln is protected with a Trt (trityl) protecting group
  SITE 20: note = Side chain of Lys is protected with a Boc (tert-butyloxycarbonyl) protecting group
  SITE 21: note = Side chain of Glu is protected with a OtBu (tert-butyl ester) protecting group
  SITE 25: note = Side chain of Trp is protected with a Boc (tert-butyloxycarbonyl) protecting group
  SITE 28: note = Side chain of Lys is protected with a Boc (tert-butyloxycarbonyl) protecting group
  SITE 36: note = Ser is protected as a dimethylated pseudoproline (Psi(Me,Me)pro) moiety in which the oxazolidine is derived from Ser
  SITE 41: note = Ser is protected as a dimethylated pseudoproline (Psi(Me,Me)pro) moiety in which the oxazolidine is derived from Ser
  SITE 46: note = Ser is protected as a dimethylated pseudoproline (Psi(Me,Me)pro) moiety in which the oxazolidine is derived from Ser
  SITE 47: note = Side chain of Lys is modified with a bromoacetyl (BrAc) group; C-terminus of Lys is amidated as a C-terminal primary amide

SEQ ID NO: 192 - protein, 47 aa, synthetic construct; identical to SEQ ID NO: 7
  SITE 2: note = Aib
  SITE 47: note = Side chain of Lys is modified with a bromoacetyl (BrAc) group; C-terminus of Lys is amidated as a C-terminal primary amide

(19) United States
(12) Patent Application Publication
(10) Pub. No.: US 2026/0193290 A1
(43) Pub. Date: Jul. 9, 2026

(54) CONVERGENT PEPTIDE SYNTHESIS METHODS

(71) Applicant: AMGEN INC., Thousand Oaks, CA (US)

(72) Inventors: James Murray, Camarillo, CA (US); Matthew G. Beaver, Natick, MA (US); Raymond Sarksian, Glendale, CA (US); Abhishek Kothari, Bangalore (IN); Ogonna Nwajiobi, Newbury Park, CA (US); James Falsey, Thousand Oaks, CA (US)

(21) Appl. No.: 19/434,338

(22) Filed: Dec. 29, 2025

Related U.S. Application Data
(60) Provisional application No. 63/739,787, filed on Dec. 30, 2024.
(60) Provisional application No. 63/906,108, filed on Oct. 27, 2025.

(51) Int. Cl. C07K 1/10; C07K 1/02; C07K 1/04

(52) U.S. Cl. CPC C07K 1/10; C07K 1/02; C07K 1/04

(57) ABSTRACT

Convergent pathways for the preparation of a peptide by solid phase peptide synthesis or liquid phase peptide synthesis, as well as peptide fragments suitable for use in such pathways, are provided. The convergent pathways may be utilized for the synthesis of agonists of the glucagon-like peptide 1 receptor (GLP-1R) such as the peptide component of maridebart cafraglutide.

### CROSS-REFERENCE TO RELATED APPLICATIONS

This application claims the benefit of priority to U.S. Provisional Patent Application No. 63/739,787, filed Dec. 30, 2024, and U.S. Provisional Patent Application No. 63/906,108, filed Oct. 27, 2025.

### SUBMISSION OF SEQUENCE LISTING

The content of the following Sequence Listing XML is incorporated herein by reference in its entirety: file name: 10696-WO01-SEC_Seqlisting, date created: Dec. 22, 2025; size: 772,232 bytes.

### FIELD

The present disclosure describes convergent pathways for the preparation of peptides on solid supports or in solution, as well as peptide intermediate fragments useful in such convergent synthesis pathways.

### BACKGROUND

Therapeutic peptides are commonly synthesized by either solid-phase peptide synthesis (SPPS) or liquid-phase peptide synthesis (LPPS) methods, both of which rely on amide-bond forming reactions to assemble peptides (Merrifield, J. Am. Chem. Soc., 1963; El-Faham & Albericio, Chem. Rev., 2011; Behrendt et al., J. Pept. Sci., 2016). SPPS immobilizes the growing peptide chain on a solid support, facilitating stepwise addition of amino acids and simplified purification, while LPPS methods proceed without such support and typically require intermediate purification steps between coupling reactions (El-Faham & Albericio, Chem. Rev., 2011). Due to its scalability, flexibility, and compatibility with automation techniques, SPPS is the most commonly used peptide synthesis approach at commercial scale, especially for short to medium length peptides. However, LPPS and hybrid LPPS/SPPS strategies are increasingly being explored for the production of large and complex peptides.

One intrinsic drawback of traditional stepwise peptide synthesis methods-whether performed on solid support or in solution—is the cumulative loss of yield and purity as peptides become longer and more complex (El-Faham & Albericio, Chem. Rev., 2011). Issues such as incomplete coupling, side reactions (e.g., racemization or aspartimide formation), aggregation, and poor solubility make purification increasingly difficult as peptide length increases, potentially reducing overall yield and purity (Behrendt et al., J. Pept. Sci., 2016). Convergent assembly, which involves preparing shorter peptide fragments and subsequently coupling these fragments to produce a longer peptide, offers a potential solution to these challenges for some peptides (Raibaut et al., Chem. Soc. Rev., 2012; Agouridas et al., Chem. Rev., 2019). By minimizing the number of reactions per fragment and isolating problematic regions into shorter amino acid sequences, convergent synthesis approaches can improve yield, reduce side reactions, and enhance synthetic efficiency (Kent, Chem. Soc. Rev., 2009; Agouridas et al., Chem. Rev., 2019).

The utility of convergent peptide synthesis depends on how the desired peptide is divided into shorter sequences. Optimal fragment boundaries vary based on factors such as solubility, aggregation tendency, reactivity at coupling junctions, and susceptibility to side reactions like epimerization, making reliable in silico predictions challenging (Behrendt et al., J. Pept. Sci., 2016; Raibaut et al., Chem. Soc. Rev., 2012). Additionally, coupling efficiency is sensitive to the local amino acid composition near the coupling site, often requiring customized strategies-such as modified reaction conditions or specific protecting groups-tailored to each individual sequence (Agouridas et al., Chem. Rev., 2019; Malins & Payne, Curr. Opin. Chem. Biol., 2014; Haase et al., Angew. Chem. Int. Ed., 2008; Chen et al., Angew. Chem. Int. Ed., 2008).

Thus, a need exists in the art for new methods of preparing peptides (e.g., glucagon-like peptide 1 (GLP-1) receptor agonist peptides) using convergent approaches, including solid phase and liquid phase convergent peptide synthesis strategies for preparing the peptide component of maridebart cafraglutide, as well as peptide intermediate fragments useful in such approaches.

### SUMMARY

Solid phase and liquid phase convergent peptide synthesis processes for the preparation of the peptide component of maridebart cafraglutide (also called MariTide or AMG 133), an agonist of the glucagon-like peptide 1 receptor (GLP-1R) and an antagonist of the glucose-dependent insulinotropic polypeptide receptor (GIPR) that is currently under investigation for the treatment of obesity and related conditions (Jastreboff, N Engl J Med, 2025), as well as peptide fragments thereof, are generally described herein. The convergent synthesis processes described herein offer several advantages over non-convergent processes for preparing a peptide component of maridebart cafraglutide, such as higher yield, reduced processing time, reduced solvent and/or reagent usage, and the ability to run reactions without the need for specialized equipment.

A peptide component of maridebart cafraglutide comprises the amino acid sequence of SEQ ID NO: 7, which is alternatively referred to as “Peptide A” herein.

One aspect of the present disclosure provides peptide fragments useful in the convergent preparation of Peptide A. In some embodiments, the present disclosure provides a peptide having a sequence of His-Aib-Glu-Gly-Thr-Phe-Thr-Ser-Asp-Tyr-Ser-Ser (SEQ ID NO: 1) (“Fragment 1”) or a protected variant thereof. In some embodiments, the present disclosure provides a peptide having a sequence of His-Aib-Glu-Gly-Thr-Phe-Thr-Ser-Asp-Tyr-Ser-Ser-Tyr (SEQ ID NO: 11) (“Fragment 1A”) or a protected variant thereof. In some embodiments, the present disclosure provides a peptide having a sequence of Tyr-Leu-Glu-Glu-Gln-Ala-Ala-Lys-Glu-Phe-Ile-Ala (SEQ ID NO: 2) (“Fragment 2”) or a protected variant thereof. In some embodiments, the present disclosure provides a peptide having a sequence of Leu-Glu-Glu-Gln-Ala-Ala-Lys-Glu-Phe-Ile (SEQ ID NO: 12) (“Fragment 2A”) or a protected variant thereof. In some embodiments, the present disclosure provides a peptide having a sequence of Tyr-Leu-Glu-Glu-Gln-Ala-Ala-Lys-Glu-Phe-Ile (SEQ ID NO: 16) (“Fragment 2B”) or a protected variant thereof. In some embodiments, the present disclosure provides a peptide having a sequence of Leu-Glu-Glu-Gln-Ala-Ala-Lys-Glu-Phe-Ile-Ala (SEQ ID NO: 17) (“Fragment 2C”) or a protected variant thereof. In some embodiments, the present disclosure provides a peptide having a sequence of Trp-Leu-Val-Lys-Gly-Gly-Gly-Gly-Gly-Gly-Gly (SEQ ID NO: 3) (“Fragment 3”) or a protected variant thereof. In some embodiments, the present disclosure provides a peptide having a sequence of Ala-Trp-Leu-Val-Lys-Gly-Gly-Gly-Gly-Gly-Gly-Gly (SEQ ID NO: 13) (“Fragment 3A”) or a protected variant thereof. In some embodiments, the present disclosure provides a peptide having a sequence of Ser-Gly-Gly-Gly-Gly-Ser-Gly-Gly-Gly-Gly-Ser-Lys (SEQ ID NO: 4) (“Fragment 4”) or a protected variant thereof. In some embodiments, the present disclosure provides a peptide having a sequence of Ala-Trp-Leu-Val-Lys-Gly-Gly-Gly (SEQ ID NO: 5) (“Fragment 5”) or a protected variant thereof. In some embodiments, the present disclosure provides a peptide having a sequence of Gly-Gly-Gly-Gly-Ser-Gly-Gly-Gly-Gly-Ser-Gly-Gly-Gly-Gly-Ser-Lys (SEQ ID NO: 6) (“Fragment 6”) or a protected variant thereof. In some embodiments, the present disclosure provides a peptide having a sequence of Ala-Trp-Leu-Val-Lys-Gly-Gly-Gly-Gly-Gly-Gly-Gly-Ser-Gly-Gly-Gly-Gly-Ser-Gly-Gly-Gly-Gly-Ser-Lys (SEQ ID NO: 9) (“Peptide B”) or a protected variant thereof. In some embodiments, the present disclosure provides a peptide having a sequence of Leu-Glu-Glu-Gln-Ala-Ala-Lys-Glu-Phe-Ile-Ala-Trp-Leu-Val-Lys-Gly-Gly-Gly-Gly-Gly-Gly-Gly-Ser-Gly-Gly-Gly-Gly-Ser-Gly-Gly-Gly-Gly-Ser-Lys (SEQ ID NO: 10) (“Peptide C”) or a protected variant thereof. In some embodiments, the present disclosure provides a peptide having a sequence of His-Aib-Glu-Gly-Thr-Phe-Thr-Ser-Asp-Tyr-Ser-Ser-Tyr-Leu-Glu-Glu-Gln-Ala-Ala-Lys-Glu-Phe-Ile (SEQ ID NO: 15) (“Peptide D”) or a protected variant thereof. In some embodiments, the present disclosure provides a peptide having a sequence of Trp-Leu-Val-Lys-Gly-Gly-Gly-Gly-Gly-Gly-Gly-Ser-Gly-Gly-Gly-Gly-Ser-Gly-Gly-Gly-Gly-Ser-Lys (SEQ ID NO: 8) (“Peptide E”) or a protected variant thereof. In some embodiments, the present disclosure provides a peptide having a sequence of Tyr-Leu-Glu-Glu-Gln-Ala-Ala-Lys-Glu-Phe-Ile-Ala-Trp-Leu-Val-Lys-Gly-Gly-Gly-Gly-Gly-Gly-Gly-Ser-Gly-Gly-Gly-Gly-Ser-Gly-Gly-Gly-Gly-Ser-Lys (SEQ ID NO: 14) (“Peptide F”) or a protected variant thereof. In some embodiments, a protected variant described herein has a protected (such as, e.g., an Fmoc-, Boc-, or Trt-protected) N-terminus. In some embodiments, a protected variant of Fragment 4, Fragment 6, Peptide B, Peptide C, Peptide E, or Peptide F described herein comprises a C-terminal lysine residue with a side chain protecting group that can be selectively removed (i.e., is orthogonal to any other side chain or termini protecting group(s), such as, e.g., a 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), benzyloxycarbonyl (Cbz), or chlorobenzyloxycarbonyl(2-Cl—Z) protecting group) to enable selective modification of the s-amino group of the C-terminal Lys residue.

In certain embodiments, “having a sequence” may be replaced by “consisting of a sequence.” In addition, in some embodiments, when a peptide or protected variant thereof is described as “comprising a sequence” (or its cognates), the peptide or protected variant thereof can alternatively be described as “having a sequence” (or its cognates). Additionally, in some embodiments, any of the peptides and protected variants described herein may be isolated peptides or protected variants, respectively. In some embodiments, a peptide or protected variant described herein (e.g., an isolated or protected variant described herein) is in the form of a salt. In some embodiments, a peptide or protected variant described herein (e.g., an isolated or protected variant described herein) is in the form of an acid-addition salt (e.g., an HCl, acetate, or TFA salt). In some embodiments, a peptide or protected variant described herein (e.g., an isolated or protected variant described herein) is in the form of a carboxylate salt (e.g., a sodium or triethylammonium salt).

Another aspect of the present disclosure provides a method of preparing Peptide B or a protected variant thereof, comprising coupling a protected variant of Fragment 5 with a protected variant of Fragment 6 to form a protected variant of Peptide B, wherein Peptide B comprises the amino acid sequence of SEQ ID NO: 9, Fragment 5 comprises the amino acid sequence of SEQ ID NO: 5, and Fragment 6 comprises the amino acid sequence of SEQ ID NO: 6.

Still another aspect of the present disclosure provides a method of preparing Peptide B or a protected variant thereof, comprising coupling a protected variant of Fragment 3A with a protected variant of Fragment 4 to form a protected variant of Peptide B, wherein Peptide B comprises the amino acid sequence of SEQ ID NO: 9, Fragment 3A comprises the amino acid sequence of SEQ ID NO: 13, and Fragment 4 comprises the amino acid sequence of SEQ ID NO: 4.

Another aspect of the present disclosure provides a method of preparing Peptide E or a protected variant thereof, comprising coupling a protected variant of Fragment 3 with a protected variant of Fragment 4 to form a protected variant of Peptide E, wherein Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8.

Still another aspect of the present disclosure provides a method of preparing Peptide B or a protected variant thereof, comprising coupling a protected variant of Peptide E with an N-protected alanine to form a protected variant of Peptide B, wherein Peptide E comprises the amino acid sequence of SEQ ID NO: 8 and Peptide B comprises the amino acid sequence of SEQ ID NO: 9.

Yet another aspect of the present disclosure provides a method of preparing Peptide B or a protected variant thereof, comprising:
coupling or having coupled a protected variant of Fragment 3 with a protected variant of Fragment 4 to form a first protected variant of Peptide E, wherein the protected variant of Fragment 3 has a free carboxyl group at the C-terminus and a protected (preferably Fmoc-protected) N-terminus, the protected variant of Fragment 4 has a free amino group at the N-terminus, Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8;
selectively deprotecting or having selectively deprotected the N-terminus of the first protected variant of Peptide E to form a second protected variant of Peptide E with a free amino group at the N-terminus; and
coupling or having coupled the second protected variant of Peptide E with an N-protected alanine to form a protected variant of Peptide B, wherein Peptide B comprises the amino acid sequence of SEQ ID NO: 9.

Another aspect of the present disclosure provides a method of preparing Peptide D or a protected variant thereof, comprising coupling a protected variant of Fragment 1 with a protected variant of Fragment 2B in to form a protected variant of Peptide D, wherein Peptide D comprises the amino acid sequence of SEQ ID NO: 15, Fragment 1 comprises the amino acid sequence of SEQ ID NO: 1, and Fragment 2B comprises the amino acid sequence of SEQ ID NO: 16.

Still another aspect of the present disclosure provides a method of preparing Peptide A or a protected variant thereof, comprising coupling a protected variant of Peptide B with a protected variant of Peptide D to form a protected variant of Peptide A, wherein Peptide B comprises the amino acid sequence of SEQ ID NO: 9, Peptide D comprises the amino acid sequence of SEQ ID NO: 15, and Peptide A comprises the amino acid sequence of SEQ ID NO: 7. In some embodiments, the method further comprises deprotecting the protected variant of Peptide A to isolate Peptide A. In some embodiments, the method further comprises adding a bromoacetyl moiety to the Lys at the C-terminus of Peptide A or a protected variant thereof.

Yet another aspect of the present disclosure provides a method of preparing Peptide A or a protected variant thereof, comprising:
(A)
(i) (a) coupling or having coupled a protected variant of Fragment 3 with a protected variant of Fragment 4 to form a first protected variant of Peptide E, wherein the protected variant of Fragment 3 has a free carboxyl group at the C-terminus and a protected (preferably Fmoc-protected) N-terminus, the protected variant of Fragment 4 has a free amino group at the N-terminus, Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8; (b) selectively deprotecting or having selectively deprotected the N-terminus of the first protected variant of Peptide E from step (A) (i) (a) to form a second protected variant of Peptide E with a free amino group at the N-terminus; (c) coupling or having coupled the second protected variant of Peptide E from step (A) (i) (b) with an N-protected alanine to form a first protected variant of Peptide B, wherein Peptide B comprises the amino acid sequence of SEQ ID NO: 9; and (d) selectively deprotecting or having selectively deprotected the N-terminus of the first protected variant of Peptide B from step (A) (i) (c) to form a second protected variant of Peptide B with a free amino group at the N-terminus; OR
(ii) (a) coupling or having coupled a protected variant of Fragment 3A with a protected variant of Fragment 4 to form a first protected variant of Peptide B, wherein the protected variant of Fragment 3A has a free carboxyl group at the C-terminus and a protected (preferably Fmoc-protected) N-terminus, the protected variant of Fragment 4 has a free amino group at the N-terminus, Fragment 3A comprises the amino acid sequence of SEQ ID NO: 13, Fragment 4 comprises the amino acid sequence of SEQ ID NO: 4, and Peptide B comprises the amino acid sequence of SEQ ID NO: 9; and (b) selectively deprotecting or having selectively deprotected the N-terminus of the first protected variant of Peptide B from step (A) (ii) (a) to form a second protected variant of Peptide B with a free amino group at the N-terminus; OR
(iii) (a) coupling or having coupled a protected variant of Fragment 5 with a protected variant of Fragment 6 to form a first protected variant of Peptide B, wherein the protected variant of Fragment 5 has a free carboxyl group at the C-terminus and a protected (preferably Trt- or Fmoc-protected, more preferably Trt-protected) N-terminus, the protected variant of Fragment 6 has a free amino group at the N-terminus, Fragment 5 comprises the amino acid sequence of SEQ ID NO: 5, Fragment 6 comprises the amino acid sequence of SEQ ID NO: 6, and Peptide B comprises the amino acid sequence of SEQ ID NO: 9; and (b) selectively deprotecting or having selectively deprotected the N-terminus of the first protected variant of Peptide B from step (A) (iii) (a) to form a second protected variant of Peptide B with a free amino group at the N-terminus;
(B) coupling or having coupled a protected variant of Fragment 1 with a protected variant of Fragment 2B to form a protected variant of Peptide D, wherein the protected variant of Fragment 1 has a free carboxyl group at the C-terminus and a protected (preferably Boc-protected) N-terminus, the protected variant of Fragment 2B has a free amino group at the N-terminus, Fragment 1 comprises the amino acid sequence of SEQ ID NO: 1, Fragment 2B comprises the amino acid sequence of SEQ ID NO: 16, and Peptide D comprises the amino acid sequence of SEQ ID NO: 15; and
(C) coupling or having coupled the second protected variant of Peptide B from step (A) with the protected variant of Peptide D from step (B) to form a protected variant of Peptide A, wherein Peptide A comprises the amino acid sequence of SEQ ID NO: 7.

In some embodiments, the method further comprises deprotecting or having deprotected the protected variant of Peptide A to isolate Peptide A.

In some embodiments, the method further comprises adding or having added a bromoacetyl moiety to the Lys at the C-terminus of Peptide A or a protected variant thereof.

Another aspect of the present disclosure provides a method of preparing Peptide C or a protected variant thereof, comprising coupling a protected variant of Fragment 2A with a protected variant of Peptide B to form a protected variant of Peptide C, wherein Fragment 2A comprises the amino acid sequence of SEQ ID NO: 12, Peptide B comprises the amino acid sequence of SEQ ID NO: 9, and Peptide C comprises the amino acid sequence of SEQ ID NO: 10.

Still another aspect of the present disclosure provides a method of preparing Peptide C or a protected variant thereof, comprising coupling a protected variant of Fragment 2C with a protected variant of Peptide E to form a protected variant of Peptide C, wherein Fragment 2C comprises the amino acid sequence of SEQ ID NO: 17, Peptide E comprises the amino acid sequence of SEQ ID NO: 8, and Peptide C comprises the amino acid sequence of SEQ ID NO: 10.

Yet another aspect of the present disclosure provides a method of preparing Peptide A or a protected variant thereof, comprising coupling a protected variant of Fragment 1A with a protected variant of Peptide C to form a protected variant of Peptide A, wherein Fragment 1A comprises the amino acid sequence of SEQ ID NO: 11, Peptide C comprises the amino acid sequence of SEQ ID NO: 10, and Peptide A comprises the amino acid sequence of SEQ ID NO: 7. In some embodiments, the method further comprises deprotecting the protected variant of Peptide A to isolate Peptide A. In some embodiments, the method further comprises adding a bromoacetyl moiety to the Lys at the C-terminus of Peptide A or a protected variant thereof.

Another aspect of the present disclosure provides a method of preparing Peptide A or a protected variant thereof, comprising:
coupling or having coupled a protected variant of Fragment 2A with a protected variant of Peptide B to form a first protected variant of Peptide C, wherein the protected variant of Fragment 2A has a free carboxyl group at the C-terminus and a protected (preferably Fmoc-protected) N-terminus, the protected variant of Peptide B has a free amino group at the N-terminus, Fragment 2A comprises the amino acid sequence of SEQ ID NO: 12, Peptide B comprises the amino acid sequence of SEQ ID NO: 9, and Peptide C comprises the amino acid sequence of SEQ ID NO: 10;
selectively deprotecting or having selectively deprotected the N-terminus of the first protected variant of Peptide C to form a second protected variant of Peptide C with a free amino group at the N-terminus; and
coupling or having coupled a protected variant of Fragment 1A with the second protected variant of Peptide C to form a protected variant of Peptide A, wherein the protected variant of Fragment 1A has a free carboxyl group at the C-terminus and a protected (preferably Boc-protected) N-terminus, Fragment 1A comprises the amino acid sequence of SEQ ID NO: 11, and Peptide A comprises the amino acid sequence of SEQ ID NO: 7.

In some embodiments, the method further comprises deprotecting or having deprotected the protected variant of Peptide A to isolate Peptide A.

In some embodiments, the method further comprises adding or having added a bromoacetyl moiety to the Lys at the C-terminus of Peptide A or a protected variant thereof.

Still another aspect of the present disclosure provides a method of preparing Peptide A or a protected variant thereof, comprising:
(A)
(i) (a) coupling or having coupled a protected variant of Fragment 3 with a protected variant of Fragment 4 to form a first protected variant of Peptide E, wherein the protected variant of Fragment 3 has a free carboxyl group at the C-terminus and a protected (preferably Fmoc-protected) N-terminus, the protected variant of Fragment 4 has a free amino group at the N-terminus, Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8; (b) selectively deprotecting or having selectively deprotected the N-terminus of the first protected variant of Peptide E from step (A) (i) (a) to form a second protected variant of Peptide E with a free amino group at the N-terminus; (c) coupling or having coupled the second protected variant of Peptide E from step (A) (i) (b) with an N-protected alanine to form a first protected variant of Peptide B, wherein Peptide B comprises the amino acid sequence of SEQ ID NO: 9; and (d) selectively deprotecting or having selectively deprotected the N-terminus of the first protected variant of Peptide B from step (A) (i) (c) to form a second protected variant of Peptide B with a free amino group at the N-terminus; OR
(ii) (a) coupling or having coupled a protected variant of Fragment 3A with a protected variant of Fragment 4 to form a first protected variant of Peptide B, wherein the protected variant of Fragment 3A has a free carboxyl group at the C-terminus and a protected (preferably Fmoc-protected) N-terminus, the protected variant of Fragment 4 has a free amino group at the N-terminus, Fragment 3A comprises the amino acid sequence of SEQ ID NO: 13, Fragment 4 comprises the amino acid sequence of SEQ ID NO: 4, and Peptide B comprises the amino acid sequence of SEQ ID NO: 9; and (b) selectively deprotecting or having selectively deprotected the N-terminus of the first protected variant of Peptide B from step (A) (ii) (a) to form a second protected variant of Peptide B with a free amino group at the N-terminus; OR
(iii) (a) coupling or having coupled a protected variant of Fragment 5 with a protected variant of Fragment 6 to form a first protected variant of Peptide B, wherein the protected variant of Fragment 5 has a free carboxyl group at the C-terminus and a protected (preferably Trt- or Fmoc-protected, more preferably Trt-protected) N-terminus, the protected variant of Fragment 6 has a free amino group at the N-terminus, Fragment 5 comprises the amino acid sequence of SEQ ID NO: 5, Fragment 6 comprises the amino acid sequence of SEQ ID NO: 6, and Peptide B comprises the amino acid sequence of SEQ ID NO: 9; and (b) selectively deprotecting or having selectively deprotected the N-terminus of the first protected variant of Peptide B from step (A) (iii) (a) to form a second protected variant of Peptide B with a free amino group at the N-terminus;
(B) (i) coupling or having coupled a protected variant of Fragment 2A with the second protected variant of Peptide B from step (A) to form a first protected variant of Peptide C, wherein the protected variant of Fragment 2A has a free carboxyl group at the C-terminus and a protected (preferably Fmoc-protected) N-terminus, Fragment 2A comprises the amino acid sequence of SEQ ID NO: 12, and Peptide C comprises the amino acid sequence of SEQ ID NO: 10; and (ii) selectively deprotecting or having selectively deprotected the N-terminus of the first protected variant of Peptide C from step (B) (i) to form a second protected variant of Peptide C with a free amino group at the N-terminus; and
(C) coupling or having coupled a protected variant of Fragment 1A with the second protected variant of Peptide C from step (B) (ii) to form a protected variant of Peptide A, wherein the protected variant of Fragment 1A has a free carboxyl group at the C-terminus and a protected (preferably Boc-protected) N-terminus, Fragment 1A comprises the amino acid sequence of SEQ ID NO: 11, and Peptide A comprises the amino acid sequence of SEQ ID NO: 7.

In some embodiments, the method further comprises deprotecting or having deprotected the protected variant of Peptide A to isolate Peptide A.

In some embodiments, the method further comprises adding or having added a bromoacetyl moiety to the Lys at the C-terminus of Peptide A or a protected variant thereof.

Yet another aspect of the present disclosure provides a method of preparing Peptide F or a protected variant thereof, comprising coupling a protected variant of Fragment 2 with a protected variant of Peptide E to form a protected variant of Peptide F, wherein Fragment 2 comprises the amino acid sequence of SEQ ID NO: 2, Peptide E comprises the amino acid sequence of SEQ ID NO: 8, and Peptide F comprises the amino acid sequence of SEQ ID NO: 14.

Still another aspect of the present disclosure provides a method of preparing Peptide A or a protected variant thereof, comprising coupling a protected variant of Fragment 1 with a protected variant of Peptide F to form a protected variant of Peptide A, wherein Fragment 1 comprises the amino acid sequence of SEQ ID NO: 1, Peptide F comprises the amino acid sequence of SEQ ID NO: 14, and Peptide A comprises the amino acid sequence of SEQ ID NO: 7. In some embodiments, the method further comprises deprotecting the protected variant of Peptide A to isolate Peptide A. In some embodiments, the method further comprises adding a bromoacetyl moiety to the Lys at the C-terminus of Peptide A or a protected variant thereof.

Another aspect of the present disclosure provides a method of preparing Peptide A or a protected variant thereof, comprising:
coupling or having coupled a protected variant of Fragment 2 with a protected variant of Peptide E to form a first protected variant of Peptide F, wherein the protected variant of Fragment 2 has a free carboxyl group at the C-terminus and a protected (preferably Fmoc-protected) N-terminus, the protected variant of Peptide E has a free amino group at the N-terminus, Fragment 2 comprises the amino acid sequence of SEQ ID NO: 2, Peptide E comprises the amino acid sequence of SEQ ID NO: 8, and Peptide F comprises the amino acid sequence of SEQ ID NO: 14;
selectively deprotecting or having selectively deprotected the N-terminus of the first protected variant of Peptide F to form a second protected variant of Peptide F with a free amino group at the N-terminus;
coupling or having coupled a protected variant of Fragment 1 with the second protected variant of Peptide F to form a protected variant of Peptide A, wherein the protected variant of Fragment 1 has a free carboxyl group at the C-terminus and a protected (preferably Boc-protected) N-terminus, Fragment 1 comprises the amino acid sequence of SEQ ID NO: 1, and Peptide A comprises the amino acid sequence of SEQ ID NO: 7.

In some embodiments, the method further comprises deprotecting or having deprotected the protected variant of Peptide A to isolate Peptide A.

In some embodiments, the method further comprises adding or having added a bromoacetyl moiety to the Lys at the C-terminus of Peptide A or a protected variant thereof.

Another aspect of the present disclosure provides a method of preparing Peptide A or a protected variant thereof, comprising:
coupling or having coupled a protected variant of Fragment 3 with a protected variant of Fragment 4 to form a first protected variant of Peptide E, wherein the protected variant of Fragment 3 has a free carboxyl group at the C-terminus and a protected (preferably Fmoc-protected) N-terminus, the protected variant of Fragment 4 has a free amino group at the N-terminus, Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8;
selectively deprotecting or having selectively deprotected the N-terminus of the first protected variant of Peptide E to form a second protected variant of Peptide E with a free amino group at the N-terminus;
coupling or having coupled a protected variant of Fragment 2 with the second protected variant of Peptide E to form a first protected variant of Peptide F, wherein the protected variant of Fragment 2 has a free carboxyl group at the C-terminus and a protected (preferably Fmoc-protected) N-terminus, Fragment 2 comprises the amino acid sequence of SEQ ID NO: 2, and Peptide F comprises the amino acid sequence of SEQ ID NO: 14;
selectively deprotecting or having selectively deprotected the N-terminus of the first protected variant of Peptide F to form a second protected variant of Peptide F with a free amino group at the N-terminus; and
coupling or having coupled a protected variant of Fragment 1 with the second protected variant of Peptide F to form a protected variant of Peptide A, wherein the protected variant of Fragment 1 has a free carboxyl group at the C-terminus and a protected (preferably Boc-protected) N-terminus, Fragment 1 comprises the amino acid sequence of SEQ ID NO: 1, and Peptide A comprises the amino acid sequence of SEQ ID NO: 7.

In some embodiments, the method further comprises deprotecting or having deprotected the protected variant of Peptide A to isolate Peptide A.

In some embodiments, the method further comprises adding or having added a bromoacetyl moiety to the Lys at the C-terminus of Peptide A or a protected variant thereof.

Still another aspect of the present disclosure provides a method of preparing Peptide A or a protected variant thereof, comprising:
coupling or having coupled a protected variant of Fragment 3 with a protected variant of Fragment 4 to form a first protected variant of Peptide E, wherein the protected variant of Fragment 3 has a free carboxyl group at the C-terminus and a protected (preferably Fmoc-protected) N-terminus, the protected variant of Fragment 4 has a free amino group at the N-terminus, Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8;
selectively deprotecting or having selectively deprotected the N-terminus of the first protected variant of Peptide E to form a second protected variant of Peptide E with a free amino group at the N-terminus;
coupling or having coupled a protected variant of Fragment 2C with the second protected variant of Peptide E to form a first protected variant of Peptide C, wherein the protected variant of Fragment 2C has a free carboxyl group at the C-terminus and a protected (preferably Fmoc-protected) N-terminus, Fragment 2C comprises the amino acid sequence of SEQ ID NO: 17, and Peptide C comprises the amino acid sequence of SEQ ID NO: 10;
selectively deprotecting or having selectively deprotected the N-terminus of the first protected variant of Peptide C to form a second protected variant of Peptide C with a free amino group at the N-terminus; and
coupling or having coupled a protected variant of Fragment 1A with the second protected variant of Peptide C to form a protected variant of Peptide A, wherein the protected variant of Fragment 1A has a free carboxyl group at the C-terminus and a protected (preferably Boc-protected) N-terminus, Fragment 1A comprises the amino acid sequence of SEQ ID NO: 11, and Peptide A comprises the amino acid sequence of SEQ ID NO: 7.

In some embodiments, the method further comprises deprotecting or having deprotected the protected variant of Peptide A to isolate Peptide A.

In some embodiments, the method further comprises adding or having added a bromoacetyl moiety to the Lys at the C-terminus of Peptide A or a protected variant thereof.

Yet another aspect of the present disclosure provides a method of preparing Peptide A or a protected variant thereof, comprising:
coupling or having coupled a protected variant of Fragment 3A with a protected variant of Fragment 4 to form a first protected variant of Peptide B, wherein the protected variant of Fragment 3A has a free carboxyl group at the C-terminus and a protected (preferably Fmoc-protected) N-terminus, the protected variant of Fragment 4 has a free amino group at the N-terminus, Fragment 3A comprises the amino acid sequence of SEQ ID NO: 13, Fragment 4 comprises the amino acid sequence of SEQ ID NO: 4, and Peptide B comprises the amino acid sequence of SEQ ID NO: 9;
selectively deprotecting or having selectively deprotected the N-terminus of the first protected variant of Peptide B to form a second protected variant of Peptide B with a free amino group at the N-terminus;
coupling or having coupled a protected variant of Fragment 2A with the second protected variant of Peptide B to form a first protected variant of Peptide C, wherein the protected variant of Fragment 2A has a free carboxyl group at the C-terminus and a protected (preferably Fmoc-protected) N-terminus, Fragment 2A comprises the amino acid sequence of SEQ ID NO: 12, and Peptide C comprises the amino acid sequence of SEQ ID NO: 10;
selectively deprotecting or having selectively deprotected the N-terminus of the first protected variant of Peptide C to form a second protected variant of Peptide C with a free amino group at the N-terminus; and
coupling or having coupled a protected variant of Fragment 1A with the second protected variant of Peptide C to form a protected variant of Peptide A, wherein the protected variant of Fragment 1A has a free carboxyl group at the C-terminus and a protected (preferably Boc-protected) N-terminus, Fragment 1A comprises the amino acid sequence of SEQ ID NO: 11, and Peptide A comprises the amino acid sequence of SEQ ID NO: 7.

In some embodiments, the method further comprises deprotecting or having deprotected the protected variant of Peptide A to isolate Peptide A.

In some embodiments, the method further comprises adding or having added a bromoacetyl moiety to the Lys at the C-terminus of Peptide A or a protected variant thereof.

Another aspect of the present disclosure provides a method of preparing Peptide A having a sequence of His-Aib-Glu-Gly-Thr-Phe-Thr-Ser-Asp-Tyr-Ser-Ser-Tyr-Leu-Glu-Glu-Gln-Ala-Ala-Lys-Glu-Phe-Ile-Ala-Trp-Leu-Val-Lys-Gly-Gly-Gly-Gly-Gly-Gly-Gly-Ser-Gly-Gly-Gly-Gly-Ser-Gly-Gly-Gly-Gly-Ser-Lys (SEQ ID NO: 7) (“Peptide A”), or a protected variant thereof, comprising:
(A)
(i) (a) reacting a protected variant of Fragment 3 with a protected variant of Fragment 4 under solid phase peptide coupling conditions to form a protected variant of Peptide E (SEQ ID NO: 8), wherein the protected variant of Fragment 4 is on solid support prior to the reacting and the protected variant of Peptide E is on solid support after the reacting; and
(b) reacting a protected variant of Peptide E with a protected alanine under solid phase peptide coupling conditions to form a protected variant of Peptide B (SEQ ID NO: 9), wherein the protected variant of Peptide B is on solid support; OR
(ii) reacting a protected variant of Fragment 3A with a protected variant of Fragment 4 under solid phase peptide coupling conditions to form a protected variant of Peptide B (SEQ ID NO: 9), wherein the protected variant of Fragment 4 is on solid support prior to the reacting and the protected variant of Peptide Bis on solid support after the reacting; OR
(iii) reacting a protected variant of Fragment 5 with a protected variant of Fragment 6 under solid phase peptide coupling conditions to form a protected variant of Peptide B (SEQ ID NO: 9), wherein the protected variant of Fragment 6 is on solid support prior to the reacting and the protected variant of Peptide B is on solid support after the reacting;
(B) reacting a protected variant of Fragment 2A (SEQ ID NO: 12) with the protected variant of Peptide B from step (A) under solid phase peptide coupling conditions to form a protected variant of Peptide C (SEQ ID NO: 10), wherein the protected variant of Peptide C is on solid support after the reacting;
(C) reacting a protected variant of Fragment 1A (SEQ ID NO: 11) with the protected variant of Peptide C from step (B) under solid phase peptide coupling conditions to form a protected variant of Peptide A, wherein the protected variant of Peptide A is on solid support after the reacting;
(D) optionally deprotecting and cleaving the protected variant of Peptide A from the solid support to isolate Peptide A; and
(E) optionally adding a bromoacetyl moiety to the Lys at the C-terminus of Peptide A.

In some embodiments, the present disclosure describes a method of preparing Peptide A or a protected variant thereof, comprising:
reacting a protected variant of Fragment 3 with a protected variant of Fragment 4 under solid phase peptide coupling conditions to form a protected variant of Peptide E, wherein Fragment 3 comprises the amino acid sequence of SEQ ID NO: 3, Fragment 4 comprises the amino acid sequence of SEQ ID NO: 4, and Peptide E comprises the amino acid sequence of 8, and further wherein the protected variant of Fragment 4 is on solid support prior to the reacting and the protected variant of Peptide E is on solid support after the reacting;
reacting the protected variant of Peptide E with a protected alanine under solid phase peptide coupling conditions to form a protected variant of Peptide B, wherein Peptide B comprises the amino acid sequence of SEQ ID NO: 9, and further wherein the protected variant of Peptide B is on solid support after the reacting;
reacting a protected variant of Fragment 2A with the protected variant of Peptide B under solid phase peptide coupling conditions to form a protected variant of Peptide C, wherein Fragment 2A comprises the amino acid sequence of SEQ ID NO: 12 and Peptide C comprises the amino acid sequence of SEQ ID NO: 10, and further wherein the protected variant of Peptide C is on solid support after the reacting;
reacting a protected variant of Fragment 1A with the protected variant of Peptide C under solid phase peptide coupling conditions to form a protected variant of Peptide A, wherein Fragment 1A comprises the amino acid sequence of SEQ ID NO: 11 and Peptide A comprises the amino acid sequence of SEQ ID NO: 7, and further wherein the protected variant of Peptide A is on solid support after the reacting; and deprotecting and cleaving the protected variant of Peptide A from the solid support to isolate Peptide A.

In some embodiments, the method further comprises adding a bromoacetyl moiety to the Lys at the C-terminus of Peptide A.

In some embodiments, the present disclosure describes a method of preparing Peptide A or a protected variant thereof, comprising:
reacting a protected variant of Fragment 3A with a protected variant of Fragment 4 under solid phase peptide coupling conditions to form a protected variant of Peptide B, wherein Fragment 3A comprises the amino acid sequence of SEQ ID NO: 13, Fragment 4 comprises the amino acid sequence of SEQ ID NO: 4, and Peptide B comprises the amino acid sequence of SEQ ID NO:9, and further wherein the protected variant of Fragment 4 is on solid support prior to the reacting and the protected variant of Peptide B is on solid support after the reacting;

reacting a protected variant of Fragment 2A with the protected variant of Peptide B under solid phase peptide coupling conditions to form a protected variant of Peptide C, wherein Fragment 2A comprises the amino acid sequence of SEQ ID NO: 12 and Peptide C comprises the amino acid sequence of SEQ ID NO: 10, and further wherein the protected variant of Peptide C is on solid support after the reacting;

reacting a protected variant of Fragment 1A with the protected variant of Peptide C under solid phase peptide coupling conditions to form a protected variant of Peptide A, wherein Fragment 1A comprises the amino acid sequence of SEQ ID NO: 11 and Peptide A comprises the amino acid sequence of SEQ ID NO: 7, and further wherein the protected variant of Peptide A is on solid support after the reacting; and deprotecting and cleaving the protected variant of Peptide A from the solid support to isolate Peptide A.

In some embodiments, the method further comprises adding a bromoacetyl moiety to the Lys at the C-terminus of Peptide A.

In some embodiments, the present disclosure describes a method of preparing Peptide A or a protected variant thereof, comprising:
reacting a protected variant of Fragment 5 with a protected variant of Fragment 6 under solid phase peptide coupling conditions to form a protected variant of Peptide B, wherein Fragment 5 comprises the amino acid sequence of SEQ ID NO: 5, Fragment 6 comprises the amino acid sequence of SEQ ID NO: 6, and Peptide B comprises the amino acid sequence of SEQ ID NO: 9, and further wherein the protected variant of Fragment 6 is on solid support prior to the reacting and the protected variant of Peptide B is on solid support after the reacting;
reacting a protected variant of Fragment 2A with the protected variant of Peptide B under solid phase peptide coupling conditions to form a protected variant of Peptide C, wherein Fragment 2A comprises the amino acid sequence of SEQ ID NO: 12 and Peptide C comprises the amino acid sequence of SEQ ID NO: 10, and further wherein the protected variant of Peptide C is on solid support after the reacting;
reacting a protected variant of Fragment 1A with the protected variant of Peptide C under solid phase peptide coupling conditions to form a protected variant of Peptide A, wherein Fragment 1A comprises the amino acid sequence of SEQ ID NO: 11 and Peptide A comprises the amino acid sequence of SEQ ID NO: 7, and further wherein the protected variant of Peptide A is on solid support after the reacting; and deprotecting and cleaving the protected variant of Peptide A from the solid support to isolate Peptide A.

In some embodiments, the method further comprises adding a bromoacetyl moiety to the Lys at the C-terminus of Peptide A.

Another aspect of the present disclosure provides a method of preparing Peptide A or a protected variant thereof, comprising:
reacting a protected variant of Fragment 3 with a protected variant of Fragment 4 under solid phase peptide coupling conditions to form a protected variant of Peptide E, wherein Fragment 3 comprises the amino acid sequence of SEQ ID NO: 3, Fragment 4 comprises the amino acid sequence of SEQ ID NO: 4, and Peptide E comprises the amino acid sequence of 8, and further wherein the protected variant of Fragment 4 is on solid support prior to the reacting and the protected variant of Peptide E is on solid support after the reacting;
reacting the protected variant of Peptide E with a protected variant of Fragment 2 under solid phase peptide coupling conditions to form a protected variant of Peptide F, wherein Peptide F comprises the amino acid sequence of SEQ ID NO: 14, and further wherein the protected variant of Peptide F is on solid support after the reacting;
reacting a protected variant of Fragment 1 with the protected variant of Peptide F under solid phase peptide coupling conditions to form a protected variant of Peptide A, wherein Fragment 1 comprises the amino acid sequence of SEQ ID NO: 1 and Peptide A comprises the amino acid sequence of SEQ ID NO: 7, and further wherein the protected variant of Peptide A is on solid support after the reacting; and
deprotecting and cleaving the protected variant of Peptide A from the solid support to isolate Peptide A.

In some embodiments, the method further comprises adding a bromoacetyl moiety to the Lys at the C-terminus of Peptide A.

Still another aspect of the present disclosure provides a method of preparing Peptide A or a protected variant thereof, comprising:
reacting a protected variant of Fragment 3 with a protected variant of Fragment 4 under solid phase peptide coupling conditions to form a protected variant of Peptide E, wherein Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8; and further wherein the protected variant of Fragment 4 is on solid support prior to the reacting and the protected variant of Peptide E is on solid support after the reacting;
reacting a protected variant of Fragment 2C with the protected variant of Peptide E on solid support under solid phase peptide coupling conditions to form a protected variant of Peptide C, wherein Fragment 2C comprises the amino acid sequence of SEQ ID NO: 17 and Peptide C comprises the amino acid sequence of SEQ ID NO: 10; and further wherein the protected variant of Peptide C is on solid support after the reacting; and
reacting a protected variant of Fragment 1A with the protected variant of Peptide C on solid support under solid phase peptide coupling conditions to form a protected variant of Peptide A, wherein Fragment 1A comprises the amino acid sequence of SEQ ID NO: 11 and Peptide A comprises the amino acid sequence of SEQ ID NO: 7 and further wherein the protected variant of Fragment 4 is on solid support prior to the reacting and the protected variant of Peptide A is on solid support after the reacting; and
deprotecting and cleaving the protected variant of Peptide A from the solid support to isolate Peptide A.

In some embodiments, the method further comprises adding a bromoacetyl moiety to the Lys at the C-terminus of Peptide A.

Another aspect of the present disclosure provides a method of preparing Peptide A or a protected variant thereof, comprising:
(A)
(i) (a) reacting a protected variant of Fragment 3 with a protected variant of Fragment 4 under solid phase peptide coupling conditions to form a protected variant of Peptide E (SEQ ID NO: 8), wherein the protected variant of Fragment 4 is on solid support prior to the reacting and the protected variant of Peptide E is on solid support after the reacting; and
(b) reacting a protected variant of Peptide E with a protected alanine under solid phase peptide coupling conditions to form a protected variant of Peptide B (SEQ ID NO: 9), wherein the protected variant of Peptide B is on solid support; OR
(ii) reacting a protected variant of Fragment 3A with a protected variant of Fragment 4 under solid phase peptide coupling conditions to form a protected variant of Peptide B (SEQ ID NO: 9), wherein the protected variant of Fragment 4 is on solid support prior to the reacting and the protected variant of Peptide B is on solid support after the reacting; OR
(iii) reacting a protected variant of Fragment 5 with a protected variant of Fragment 6 under solid phase peptide coupling conditions to form a protected variant of Peptide B (SEQ ID NO: 9), wherein the protected variant of Fragment 6 is on solid support prior to the reacting and the protected variant of Peptide B is on solid support after the reacting;
(B) reacting a protected variant of Peptide D (SEQ ID NO: 15) with the protected variant of Peptide B from step (A) under solid phase peptide coupling conditions to form a protected variant of Peptide A (SEQ ID NO: 7), wherein the protected variant of Peptide A is on solid support after the reacting;
(C) optionally deprotecting and cleaving the protected variant of Peptide A from the solid support to isolate Peptide A; and
(D) optionally adding a bromoacetyl moiety to the Lys at the C-terminus of Peptide A.

Yet another aspect of the present disclosure provides a method of preparing Peptide A or a variant thereof, comprising:
(A) coupling a protected variant of Fragment 5 with a protected variant of Fragment 6 to form a protected variant of Peptide B; and/or
(B) coupling a protected variant of Fragment 1 with a protected variant of Fragment 2B to form a protected variant of Peptide D; and/or
(C) coupling a protected variant of Peptide B with a protected variant of Peptide D to form a protected variant of Peptide A,
wherein Peptide A has the amino acid sequence of SEQ ID NO: 7, Peptide B has the amino acid sequence of SEQ ID NO: 9, Peptide D has the amino acid sequence of SEQ ID NO: 15, Fragment 1 has the amino acid sequence of SEQ ID NO: 1, Fragment 2B has the amino acid sequence of SEQ ID NO: 16, Fragment 5 has the amino acid sequence of SEQ ID NO: 5, and Fragment 6 has the amino acid sequence of SEQ ID NO: 6.

Still another aspect of the present disclosure provides a molecule selected from:

(SEQ ID NO: 166)
Boc-His(Trt)-Aib-Glu(OtBu)-Gly-Thr(tBu)-Phe-
Thr(tBu)-Ser(tBu)-Asp(OtBu)-Tyr(tBu)-Ser(tBu)-
Ser(Psi(Me,Me)Pro)-OH;
(SEQ ID NO: 30)
H-Tyr(tBu)-Leu-Glu(OtBu)-Glu(OtBu)-Gln(Trt)-Ala-
Ala-Lys(Boc)-Glu(OtBu)-Phe-Ile-OH;
(SEQ ID NO: 169)
Boc-His(Trt)-Aib-Glu(OtBu)-Gly-Thr(tBu)-Phe-
Thr(tBu)-Ser(tBu)-Asp(OtBu)-Tyr(tBu)-Ser(tBu)-
Ser(Psi(Me,Me)Pro)-Tyr(tBu)-Leu-Glu(OtBu)-
Glu(OtBu)-Gln(Trt)-Ala-Ala-Lys(Boc)-Glu(OtBu)-
Phe-Ile-OH;
(SEQ ID NO: 59)
Trt-Ala-Trp(Boc)-Leu-Val-Lys(Boc)-Gly-Gly-Gly-
OH;
(SEQ ID NO: 130)
H-Gly-Gly-Gly-Gly-Ser(Psi(Me,Me)Pro)-Gly-Gly-Gly-
Gly-Ser(Psi(Me,Me)Pro)-Gly-Gly-Gly-Gly-Ser(Psi
(Me,Me)Pro)-Lys(ivDde)-NH₂;
(SEQ ID NO: 170)
Trt-Ala-Trp(Boc)-Leu-Val-Lys(Boc)-Gly-Gly-Gly-
Gly-Gly-Gly-Gly-Ser(Psi(Me,Me)Pro)-Gly-Gly-Gly-
Gly-Ser(Psi(Me,Me)Pro)-Gly-Gly-Gly-Gly-Ser(Psi
(Me,Me)Pro)-Lys(ivDde)-NH₂;
(SEQ ID NO: 147)
H-Ala-Trp(Boc)-Leu-Val-Lys(Boc)-Gly-Gly-Gly-Gly-
Gly-Gly-Gly-Ser(Psi(Me,Me)Pro)-Gly-Gly-Gly-Gly-
Ser(Psi(Me,Me)Pro)-Gly-Gly-Gly-Gly-Ser(Psi
(Me,Me)Pro)-Lys(ivDde)-NH₂;
and
(SEQ ID NO: 171)
Boc-His(Trt)-Aib-Glu(OtBu)-Gly-Thr(tBu)-Phe-
Thr(tBu)-Ser(tBu)-Asp(OtBu)-Tyr(tBu)-Ser(tBu)-
Ser(Psi(Me,Me)Pro)-Tyr(tBu)-Leu-Glu(OtBu)-Glu
(OtBu)-Gln(Trt)-Ala-Ala-Lys(Boc)-Glu(OtBu)-Phe-
Ile-Ala-Trp(Boc)-Leu-Val-Lys(Boc)-Gly-Gly-Gly-
Gly-Gly-Gly-Gly-Ser(Psi(Me,Me)Pro)-Gly-Gly-Gly-
Gly-Ser(Psi(Me,Me)Pro)-Gly-Gly-Gly-Gly-Ser(Psi
(Me,Me)Pro)-Lys(ivDde)-NH₂,

or a salt thereof.

Another aspect of the disclosure provides a method of preparing Peptide B or a protected variant thereof, the method comprising coupling a protected variant of Fragment 5 with a protected variant of Fragment 6 in solution to form a protected variant of Peptide B, wherein Peptide B has the amino acid sequence of SEQ ID NO: 9, Fragment 5 has the amino acid sequence of SEQ ID NO: 5, and Fragment 6 has the amino acid sequence of SEQ ID NO: 6, further wherein:
the N-terminus, the side chain of the Trp residue at position 2, and the side chain of the Lys residue at position 5 of the protected variant of Fragment 5 are protected and the protected variant of Fragment 5 has a free carboxyl group at the C-terminus; and
the side chain of the Ser residue at position 5, the side chain of the Ser residue at position 10, the side chain of the Ser residue at position 15, and the side chain of the Lys residue at position 16 of the protected variant of Fragment 6 are protected, the protected variant of Fragment 6 has a free amino group at the N-terminus, and the protected variant of Fragment 6 does not have a free carboxyl group at the C-terminus.

In some embodiments, the side chain of the Trp residue at position 2 and the side chain of the Lys residue at position 5 of the protected variant of Fragment 5 are each independently protected with an acid-labile protecting group; the side chain of the Ser residue at position 5, the side chain of the Ser residue at position 10, and the side chain of the Ser residue at position 15 of the protected variant of Fragment 6 are each independently protected with an acid-labile protecting group; and the side chain of the Lys residue at position 16 of the protected variant of Fragment 6 is orthogonally protected relative to the side chain protecting groups of the protected variant of Fragment 5 and the protected variant of Fragment 6 and the N-terminal protecting group of the protected variant of Fragment 5. Additionally, in some embodiments, the N-terminal protecting group of the protected variant of Fragment 5 can be removed without removing the side chain protecting groups of the protected variant of Fragment 5 and the protected variant of Fragment 6. Furthermore, in some embodiments, the C-terminus of the protected variant of Fragment 6 is amidated as a C-terminal primary amide.

Still another aspect of the present disclosure provides a method of preparing a Peptide D or a protected variant thereof, the method comprising coupling a protected variant of Fragment 1 with a protected variant of Fragment 2B in solution to form a protected variant of Peptide D, wherein Peptide D has the amino acid sequence of SEQ ID NO: 15, Fragment 1 has the amino acid sequence of SEQ ID NO: 1, and Fragment 2B has the amino acid sequence of SEQ ID NO: 16, further wherein:
the N-terminus, the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, and the side chain of the Ser residue at position 12 of the protected variant of Fragment 1 are protected and the protected variant of Fragment 1 has a free carboxyl group at the C-terminus; and
the side chain of the Tyr residue at position 1, the side chain of the Glu residue at position 3, the side chain of the Glu residue at position 4, the side chain of the Gln residue at position 5, the side chain of the Lys residue at position 8, and the side chain of the Glu residue at position 9 of the protected variant of Fragment 2B are protected and the protected variant of Fragment 2B has a free amino group at the N-terminus.

In some embodiments, the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, and the side chain of the Ser residue at position 12 of the protected variant of Fragment 1 are each independently protected with an acid-labile protecting group; and the side chain of the Tyr residue at position 1, the side chain of the Glu residue at position 3, the side chain of the Glu residue at position 4, the side chain of the Gln residue at position 5, the side chain of the Lys residue at position 8, and the side chain of the Glu residue at position 9 of the protected variant of Fragment 2B are each independently protected with an acid-labile protecting group. Additionally, in some embodiments, the N-terminus of the protected variant of Fragment 1 is an acid-labile protecting group.

Still another aspect of the disclosure provides a method of preparing Peptide A or a variant thereof, the method comprising coupling a protected variant of Peptide B with a protected variant of Peptide D to form a protected variant of Peptide A, wherein Peptide A has the amino acid sequence of SEQ ID NO: 7, Peptide B has the amino acid sequence of SEQ ID NO: 9, and Peptide D has the amino acid sequence of SEQ ID NO: 15, further wherein:
the side chain of the Trp residue at position 2, the side chain of the Lys residue at position 5, the side chain of the Ser residue at position 13, the side chain of the Ser residue at position 18, the side chain of the Ser residue at position 23, and the side chain of the Lys residue at position 24 of the protected variant of Peptide B are protected, the protected variant of Peptide B has a free amino group at the N-terminus, and the protected variant of Peptide B does not have a free carboxyl group at the C-terminus; and
the N-terminus, the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, the side chain of the Ser residue at position 12, the side chain of the Tyr residue at position 13, the side chain of the Glu residue at position 15, the side chain of the Glu residue at position 16, the side chain of the Gln residue at position 17, the side chain of the Lys residue at position 20, and the side chain of the Glu residue at position 21 of the protected variant of Peptide D are protected and the protected variant of Peptide D has a free carboxyl group at the C-terminus.

In some embodiments, the side chain of the Trp residue at position 2, the side chain of the Lys residue at position 5, the side chain of the Ser residue at position 13, the side chain of the Ser residue at position 18, and the side chain of the Ser residue at position 23 of the protected variant of Peptide B are each independently protected with an acid-labile protecting group; the N-terminus, the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, the side chain of the Ser residue at position 12, the side chain of the Tyr residue at position 13, the side chain of the Glu residue at position 15, the side chain of the Glu residue at position 16, the side chain of the Gln residue at position 17, the side chain of the Lys residue at position 20, and the side chain of the Glu residue at position 21 of the protected variant of Peptide D are each independently protected with an acid-labile protecting group; and the side chain of the Lys residue at position 24 of the protected variant of Peptide B is orthogonally protected relative to the side chain protecting groups of the protected variant of Peptide B and the protected variant of Peptide D and the N-terminal protecting group of the protected variant of Peptide D. Furthermore, in some embodiments, the C-terminus of the protected variant of Peptide B is amidated as a C-terminal primary amide.

Other advantages and novel features of the present disclosure will become apparent from the following detailed description of various non-limiting embodiments. In cases where the present specification and a document incorporated by reference include conflicting and/or inconsistent disclosure, the present specification shall control.

### BRIEF DESCRIPTION OF THE DRAWINGS

FIGS. 1A-1D show schematic representations of a convergent solid phase synthesis route for a protected variant of Peptide A (SEQ ID NO: 179) from protected variants of Fragment 1A (SEQ ID NO: 95), Fragment 2A (SEQ ID NO: 53), Fragment 3 (SEQ ID NO: 56), alanine, and Fragment 4 (SEQ ID NO: 117) in accordance with certain embodiments of the present disclosure.

FIG. 2A depicts a reaction scheme for coupling a protected variant of Fragment 6 with an amidated C-terminus, where Fragment 6 has the amino acid sequence of SEQ ID NO: 6, and a protected variant of Fragment 5, where Fragment 5 has the amino acid sequence of SEQ ID NO: 5, to form a protected variant of Peptide B with an amidated C-terminus, where Peptide B has the amino acid sequence of SEQ ID NO: 9, in accordance with certain disclosed embodiments.

FIG. 2B provides a reaction scheme for coupling a protected variant of Fragment 6 with an amidated C-terminus, where Fragment 6 has the amino acid sequence of SEQ ID NO: 6, and a protected variant of Fragment 5, where Fragment 5 has the amino acid sequence of SEQ ID NO: 5, to form a protected variant of Peptide B with an amidated C-terminus, where Peptide B has the amino acid sequence of SEQ ID NO: 9, in accordance with certain disclosed embodiments.

FIG. 2C depicts another protected variant of Peptide B with an amidated C-terminus (SEQ ID NO: 147) synthesized in accordance with certain disclosed embodiments.

FIG. 2D provides another reaction scheme for coupling a protected variant of Fragment 6 with an amidated C-terminus (SEQ ID NO: 130) and a protected variant of Fragment 5 (SEQ ID NO: 59) to form a protected variant of Peptide B with an amidated C-terminus (SEQ ID NO: 147) in accordance with certain disclosed embodiments.

FIG. 3A shows a reaction scheme for coupling a protected variant of Fragment 1, where Fragment 1 has the amino acid sequence of SEQ ID NO: 1, and a protected variant of Fragment 2B, where Fragment 2B has the amino acid sequence of SEQ ID NO: 16, to form a protected variant of Peptide D, where Peptide D has the amino acid sequence of SEQ ID NO: 15, in accordance with certain disclosed embodiments.

FIG. 3B depicts another protected variant of Peptide D (SEQ ID NO: 157) synthesized in accordance with certain disclosed embodiments.

FIG. 3C shows another reaction scheme for coupling a protected variant of Fragment 1 (SEQ ID NO: 166) and a protected variant of Fragment 2B (SEQ ID NO: 30) to form a protected variant of Peptide D (SEQ ID NO: 169) in accordance with certain disclosed embodiments.

FIGS. 4A and 4B show a reaction scheme for coupling a protected variant of Peptide B with an amidated C-terminus (SEQ ID NO: 147) to a protected variant of Peptide D (SEQ ID NO: 169) to form a protected variant of Peptide A with an amidated C-terminus (SEQ ID NO: 171) in accordance with certain disclosed embodiments. FIGS. 4C and 4D provide a reaction scheme for coupling a protected variant of Peptide B with an amidated C-terminus (SEQ ID NO: 147) to a protected variant of Peptide D (SEQ ID NO: 168) to form a protected variant of Peptide A with an amidated C-terminus in accordance with other disclosed embodiments.

FIGS. 5A and 5B depict ivDde deprotection of a protected variant of Peptide A with an amidated C-terminus in accordance with certain disclosed embodiments.

FIG. 5C shows a partially deprotected variant of Peptide A in which the &-amino group of the C-terminal lysine residue is deprotected and the C-terminus is amidated (SEQ ID NO: 191). This molecule can be synthesized in accordance with certain embodiments of the present disclosure.

FIGS. 5D and 5E depict ivDde deprotection, bromoacetylation, and global deprotection of a protected variant of Peptide A with an amidated C-terminus in accordance with certain disclosed embodiments.

FIG. 6A depicts bromoacetylation of a partially deprotected variant of Peptide A with a C-terminal amide group in accordance with certain disclosed embodiments.

FIG. 6B shows a bromoacetylated and protected variant of Peptide A with an amidated C-terminus synthesized in accordance with certain disclosed embodiments.

### DETAILED DESCRIPTION

Provided herein are methods of synthesizing a peptide component of maridebart cafraglutide (Peptide A) and fragments thereof using a convergent peptide synthesis approach (e.g., a convergent solid phase peptide synthesis approach, a convergent liquid phase peptide synthesis approach, or a hybrid approach in which one or more couplings are performed on solid support and one or more couplings are performed in solution to obtain Peptide A or a fragment thereof). To develop efficient and scalable convergent approaches for the preparation of Peptide A, various peptide fragments and associated protecting groups, as well as various coupling conditions, were investigated.

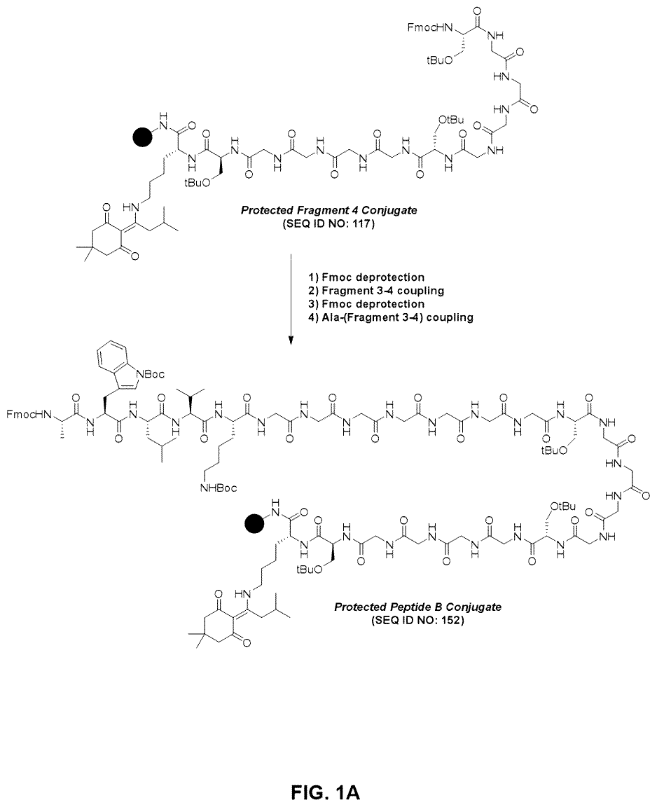

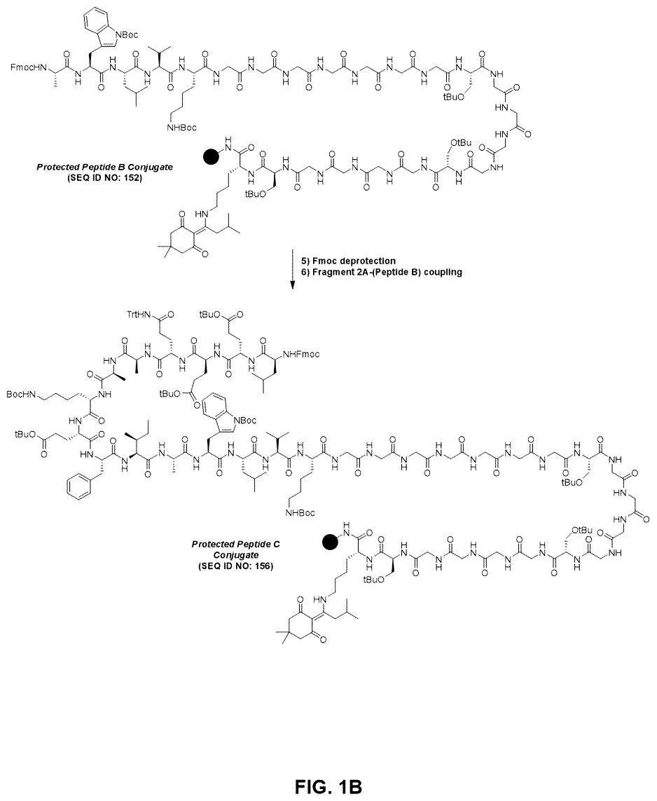

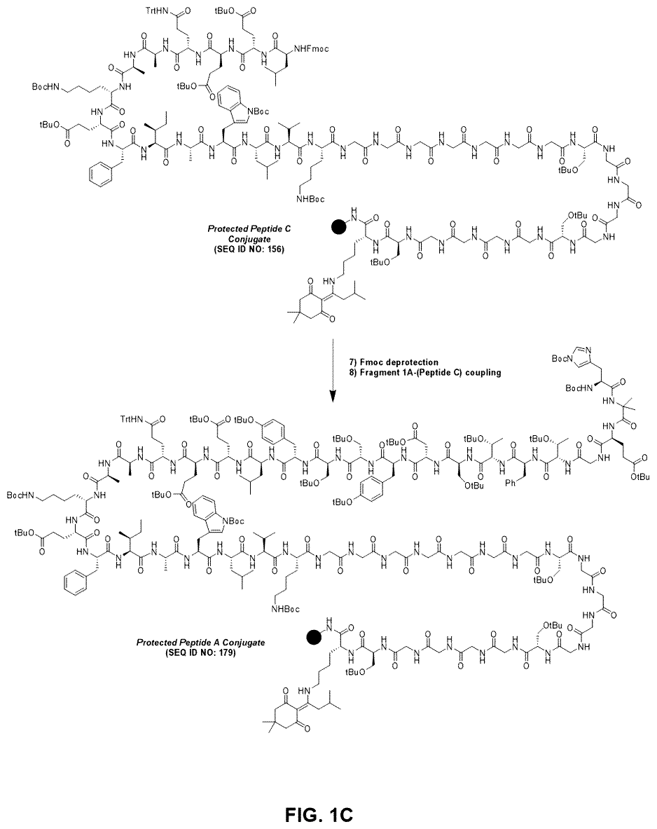

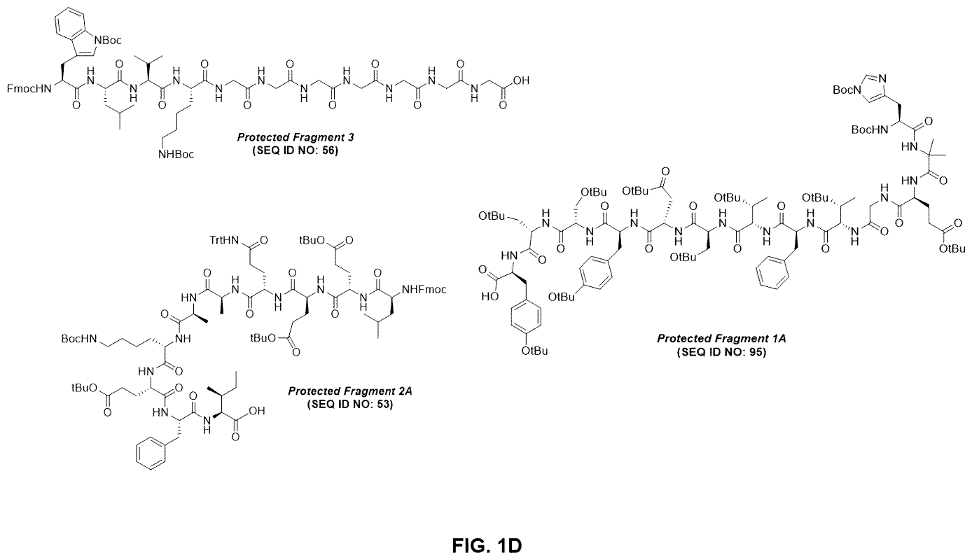

Illustratively, it was determined that, in some embodiments, Peptide A can be efficiently prepared on solid support by first coupling (i) a protected variant of Fragment 3 and a protected variant of Fragment 4 (which is on solid support) then adding a protected Ala to the N-terminus to form a protected variant of Peptide B (which is on solid support), a synthetic intermediate as shown in FIG. 1A, or (ii) a protected variant of Fragment 3A and a protected variant of Fragment 4 (which is on solid support) to form a protected variant of Peptide B (which is on solid support), a synthetic intermediate, or (iii) a protected variant of Fragment 5 and a protected variant of Fragment 6 (which is on solid support) to form a protected variant of Peptide B on solid support. Then, a protected variant of Fragment 2A can be coupled to the protected variant of Peptide B (on solid support) to form a protected variant of Peptide C on solid support, as shown in FIG. 1B. Finally, a protected variant of Fragment 1A can be coupled to the protected variant of Peptide C (on solid support) to form a protected variant of Peptide A on solid support, as shown in FIG. 1C. Additional deprotection and cleavage steps can be performed to isolate Peptide A from the solid support. Optionally, Peptide A can also be reacted to add a bromoacetyl moiety at its C-terminal Lys residue.

Solid phase synthesis conditions for each coupling step can be selected to provide desired yield and regioselectivity. Protecting groups for the protected peptide variants (i.e., peptides with one or more side-chain protecting groups to facilitate peptide synthesis) can be selected to provide reactive inertness during peptide coupling while minimizing impediments to such peptide couplings. These selections can be informed by the discussion and examples provided below. Additionally, in some embodiments of SPPS methods described herein, the N-terminus of a protected amino acid or peptide on solid support can initially be present as a free α-amino group, or the protected amino acid or peptide can initially be present in the form of an acid-addition salt (such as, e.g., an HCl or TFA salt following acidolytic deprotection). In certain embodiments in which the protected amino acid or peptide is initially in the form of an acid-addition salt, the protected amino acid or peptide on solid support can be contacted with a base (such as, e.g., a non-nucleophilic tertiary base, e.g., DIPEA, NMM, or collidine) to convert the acid-addition salt to a free base, thereby liberating an α-amino group for reaction with a coupling partner with an activated C-terminal carboxyl group.

Provided herein are methods of preparing Peptide A having a sequence of His-Aib-Glu-Gly-Thr-Phe-Thr-Ser-Asp-Tyr-Ser-Ser-Tyr-Leu-Glu-Glu-Gln-Ala-Ala-Lys-Glu-Phe-Ile-Ala-Trp-Leu-Val-Lys-Gly-Gly-Gly-Gly-Gly-Gly-Gly-Ser-Gly-Gly-Gly-Gly-Ser-Gly-Gly-Gly-Gly-Ser-Lys (SEQ ID NO: 7) (“Peptide A”), or a protected variant thereof, comprising:
(A)
   (i) (a) reacting a protected variant of Fragment 3 with a protected variant of Fragment 4 under solid phase peptide coupling conditions to form a protected variant of Peptide E (SEQ ID NO: 8), wherein the protected variant of Fragment 4 is on solid support prior to the reacting and the protected variant of Peptide E is on solid support after the reacting; and
   (b) reacting a protected variant of Peptide E with a protected alanine under solid phase peptide coupling conditions to form a protected variant of Peptide B (SEQ ID NO: 9), wherein the protected variant of Peptide B is on solid support; OR
   (ii) reacting a protected variant of Fragment 3A with a protected variant of Fragment 4 under solid phase peptide coupling conditions to form a protected variant of Peptide B (SEQ ID NO: 9), wherein the protected variant of Fragment 4 is on solid support prior to the reacting and the protected variant of Peptide B is on solid support after the reacting; OR
   (iii) reacting a protected variant of Fragment 5 with a protected variant of Fragment 6 under solid phase peptide coupling conditions to form a protected variant of Peptide B (SEQ ID NO: 9), wherein the protected variant of Fragment 6 is on solid support prior to the reacting and the protected variant of Peptide B is on solid support after the reacting;
(B) reacting a protected variant of Fragment 2A (SEQ ID NO: 12) with the protected variant of Peptide B from step (A) under solid phase peptide coupling conditions to form a protected variant of Peptide C (SEQ ID NO: 10), wherein the protected variant of Peptide C is on solid support after the reacting;
(C) reacting a protected variant of Fragment 1A (SEQ ID NO: 11) with the protected variant of Peptide C from step (B) under solid phase peptide coupling conditions to form a protected variant of Peptide A, wherein the protected variant of Peptide A is on solid support after the reacting;
(D) optionally deprotecting and cleaving the protected variant of Peptide A from the solid support to isolate Peptide A; and
(E) optionally adding a bromoacetyl moiety to the Lys at the C-terminus of Peptide A.

In some embodiments, the method comprises:
(A)
   (i) reacting a protected variant of Fragment 3 of any one of the aspects of the present disclosure with a protected variant of Fragment 4 of any one of the aspects of the present disclosure under solid phase peptide coupling conditions to form a protected variant of Peptide E (SEQ ID NO: 8), wherein the protected variant of Fragment 4 is on solid support before the reacting and the protected variant of Peptide E is on solid support after the reacting; and
   (ii) reacting the protected variant of Peptide E on solid support from step (A) (i) with a protected alanine under solid phase peptide coupling conditions to form a protected variant of Peptide B (SEQ ID NO: 9), wherein the protected variant of Peptide B is on solid support after the reacting;
(B) reacting a protected variant of Fragment 2A of any one of the aspects of the present disclosure with the protected variant of Peptide B on solid support from step (A) under solid phase peptide coupling conditions to form a protected variant of Peptide C (SEQ ID NO: 10), wherein the protected variant of Peptide C is on solid support after the reacting;
(C) reacting a protected variant of Fragment 1A of any one of the aspects of the present disclosure with the protected variant of Peptide C on solid support from step (B) under solid phase peptide coupling conditions to form a protected variant of Peptide A, wherein the protected variant of Peptide A is on solid support after the reacting;
(D) optionally deprotecting and cleaving the protected variant of Peptide A from the solid support to isolate Peptide A; and
(E) optionally adding a bromoacetyl moiety to the Lys at the C-terminus of Peptide A.

In some embodiments, the method comprises:
(A) reacting a protected variant of Fragment 3A of any one of the aspects of the present disclosure with a protected variant of Fragment 4 of any one of the aspects of the present disclosure under solid phase peptide coupling conditions to form a protected variant of Peptide B (SEQ ID NO: 9), wherein the protected variant of Fragment 4 is on solid support before the reacting and the protected variant of Peptide B is on solid support after the reacting;
(B) reacting a protected variant of Fragment 2A of any one of the aspects of the present disclosure with the protected variant of Peptide B on solid support from step (A) under solid phase peptide coupling conditions to form a protected variant of Peptide C (SEQ ID NO: 10), wherein the protected variant of Peptide C is on solid support after the reacting;
(C) reacting a protected variant of Fragment 1A of any one of the aspects of the present disclosure with the protected variant of Peptide C on solid support from step (B) under solid phase peptide coupling conditions to form a protected variant of Peptide A, wherein the protected variant of Peptide A is on solid support after the reacting;
(D) optionally deprotecting and cleaving the protected variant of Peptide A from the solid support to isolate Peptide A; and
(E) optionally adding a bromoacetyl moiety to the Lys at the C-terminus of Peptide A.

In some embodiments, the method comprises:
(A) reacting a protected variant of Fragment 5 of any one of the aspects of the present disclosure with a protected variant of Fragment 6 of any one of the aspects of the present disclosure under solid phase peptide coupling conditions to form a protected variant of Peptide B (SEQ ID NO: 9), wherein the protected variant of Fragment 6 is on solid support before the reacting and the protected variant of Peptide B is on solid support after the reacting;
(B) reacting a protected variant of Fragment 2A of any one of the aspects of the present disclosure with the protected variant of Peptide B on solid support from step (A) under solid phase peptide coupling conditions to form a protected variant of Peptide C (SEQ ID NO: 10), wherein the protected variant of Peptide C is on solid support after the reacting;
(C) reacting a protected variant of Fragment 1A of any one of the aspects of the present disclosure with the protected variant of Peptide C on solid support from step (B) under solid phase peptide coupling conditions to form a protected variant of Peptide A, wherein the protected variant of Peptide A is on solid support after the reacting;
(D) optionally deprotecting and cleaving the protected variant of Peptide A from the solid support to isolate Peptide A; and
(E) optionally adding a bromoacetyl moiety to the Lys at the C-terminus of Peptide A.

Also provided herein is a method of preparing Peptide A or a protected variant thereof, comprising:
reacting a protected variant of Fragment 3 with a protected variant of Fragment 4 under solid phase peptide coupling conditions to form a protected variant of Peptide E, wherein Fragment 3 comprises the amino acid sequence of SEQ ID NO: 3, Fragment 4 comprises the amino acid sequence of SEQ ID NO: 4, and Peptide E comprises the amino acid sequence of 8, and further wherein the protected variant of Fragment 4 is on solid support prior to the reacting and the protected variant of Peptide E is on solid support after the reacting;
reacting the protected variant of Peptide E with a protected variant of Fragment 2 under solid phase peptide coupling conditions to form a protected variant of Peptide F, wherein Peptide F comprises the amino acid sequence of SEQ ID NO: 14, and further wherein the protected variant of Peptide F is on solid support after the reacting;
reacting a protected variant of Fragment 1 with the protected variant of Peptide F under solid phase peptide coupling conditions to form a protected variant of Peptide A, wherein Fragment 1 comprises the amino acid sequence of SEQ ID NO: 1 and Peptide A comprises the amino acid sequence of SEQ ID NO: 7, and further wherein the protected variant of Peptide A is on solid support after the reacting; and
deprotecting and cleaving the protected variant of Peptide A from the solid support to isolate Peptide A.

In some embodiments, the method further comprises adding a bromoacetyl moiety to the Lys at the C-terminus of Peptide A.

Also provided herein is a method of preparing Peptide A or a protected variant thereof, comprising:
reacting a protected variant of Fragment 3 with a protected variant of Fragment 4 under solid phase peptide coupling conditions to form a protected variant of Peptide E, wherein Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8; and further wherein the protected variant of Fragment 4 is on solid support prior to the reacting and the protected variant of Peptide E is on solid support after the reacting;
reacting a protected variant of Fragment 2C with the protected variant of Peptide E on solid support under solid phase peptide coupling conditions to form a protected variant of Peptide C, wherein Fragment 2C comprises the amino acid sequence of SEQ ID NO: 17 and Peptide C comprises the amino acid sequence of SEQ ID NO: 10; and further wherein the protected variant of Peptide C is on solid support after the reacting; and
reacting a protected variant of Fragment 1A with the protected variant of Peptide C on solid support under solid phase peptide coupling conditions to form a protected variant of Peptide A, wherein Fragment 1A comprises the amino acid sequence of SEQ ID NO: 11 and Peptide A comprises the amino acid sequence of SEQ ID NO: 7 and further wherein the protected variant of Fragment 4 is on solid support prior to the reacting and the protected variant of Peptide A is on solid support after the reacting; and
deprotecting and cleaving the protected variant of Peptide A from the solid support to isolate Peptide A.

In some embodiments, the method further comprises adding a bromoacetyl moiety to the Lys at the C-terminus of Peptide A.

Also provided herein a method of preparing Peptide A or a protected variant thereof, comprising:
(A)
(i) (a) reacting a protected variant of Fragment 3 with a protected variant of Fragment 4 under solid phase peptide coupling conditions to form a protected variant of Peptide E (SEQ ID NO: 8), wherein the protected variant of Fragment 4 is on solid support prior to the reacting and the protected variant of Peptide E is on solid support after the reacting; and
(b) reacting a protected variant of Peptide E with a protected alanine under solid phase peptide coupling conditions to form a protected variant of Peptide B (SEQ ID NO: 9), wherein the protected variant of Peptide B is on solid support; OR
(ii) reacting a protected variant of Fragment 3A with a protected variant of Fragment 4 under solid phase peptide coupling conditions to form a protected variant of Peptide B (SEQ ID NO: 9), wherein the protected variant of Fragment 4 is on solid support prior to the reacting and the protected variant of Peptide B is on solid support after the reacting; OR
(iii) reacting a protected variant of Fragment 5 with a protected variant of Fragment 6 under solid phase peptide coupling conditions to form a protected variant of Peptide B (SEQ ID NO: 9), wherein the protected variant of Fragment 6 is on solid support prior to the reacting and the protected variant of Peptide B is on solid support after the reacting;
(B) reacting a protected variant of Peptide D (SEQ ID NO: 15) with the protected variant of Peptide B from step (A) under solid phase peptide coupling conditions to form a protected variant of Peptide A (SEQ ID NO: 7), wherein the protected variant of Peptide A is on solid support after the reacting;
(C) optionally deprotecting and cleaving the protected variant of Peptide A from the solid support to isolate Peptide A; and
(D) optionally adding a bromoacetyl moiety to the Lys at the C-terminus of Peptide A.

In some embodiments, the method comprises:
(A) reacting a protected variant of Fragment 3 with a protected variant of Fragment 4 under solid phase peptide coupling conditions to form a protected variant of Peptide E (SEQ ID NO: 8), wherein the protected variant of Fragment 4 is on solid support prior to the reacting and the protected variant of Peptide E is on solid support after the reacting; and reacting a protected variant of Peptide E with a protected alanine under solid phase peptide coupling conditions to form a protected variant of Peptide B (SEQ ID NO: 9), wherein the protected variant of Peptide B is on solid support;
(B) reacting a protected variant of Peptide D (SEQ ID NO: 15) with the protected variant of Peptide B from step (A) under solid phase peptide coupling conditions to form a protected variant of Peptide A (SEQ ID NO: 7), wherein the protected variant of Peptide A is on solid support after the reacting;
(C) optionally deprotecting and cleaving the protected variant of Peptide A from the solid support to isolate Peptide A; and
(D) optionally adding a bromoacetyl moiety to the Lys at the C-terminus of Peptide A.

In some embodiments, the method comprises:
(A) reacting a protected variant of Fragment 3A with a protected variant of Fragment 4 under solid phase peptide coupling conditions to form a protected variant of Peptide B (SEQ ID NO: 9), wherein the protected variant of Fragment 4 is on solid support prior to the reacting and the protected variant of Peptide B is on solid support after the reacting;
(B) reacting a protected variant of Peptide D (SEQ ID NO: 15) with the protected variant of Peptide B from step (A) under solid phase peptide coupling conditions to form a protected variant of Peptide A (SEQ ID NO: 7), wherein the protected variant of Peptide A is on solid support after the reacting;
(C) optionally deprotecting and cleaving the protected variant of Peptide A from the solid support to isolate Peptide A; and
(D) optionally adding a bromoacetyl moiety to the Lys at the C-terminus of Peptide A.

In some embodiments, the method comprises:
(A) reacting a protected variant of Fragment 5 with a protected variant of Fragment 6 under solid phase peptide coupling conditions to form a protected variant of Peptide B (SEQ ID NO: 9), wherein the protected variant of Fragment 6 is on solid support prior to the reacting and the protected variant of Peptide B is on solid support after the reacting;
(B) reacting a protected variant of Peptide D (SEQ ID NO: 15) with the protected variant of Peptide B from step (A) under solid phase peptide coupling conditions to form a protected variant of Peptide A (SEQ ID NO: 7), wherein the protected variant of Peptide A is on solid support after the reacting;
(C) optionally deprotecting and cleaving the protected variant of Peptide A from the solid support to isolate Peptide A; and
(D) optionally adding a bromoacetyl moiety to the Lys at the C-terminus of Peptide A.

Also provided herein is a method of preparing Peptide B or a protected variant thereof, comprising coupling a protected variant of Fragment 5 with a protected variant of Fragment 6 to form a protected variant of Peptide B, wherein Peptide B comprises the amino acid sequence of SEQ ID NO: 9, Fragment 5 comprises the amino acid sequence of SEQ ID NO: 5, and Fragment 6 comprises the amino acid sequence of SEQ ID NO: 6.

In some embodiments, the protected variant of Fragment 5 is coupled with the protected variant of Fragment 6 in solution. In some embodiments, the protected variant of Fragment 5 is coupled with the protected variant of Fragment 6 under liquid phase peptide coupling conditions. In some embodiments, the protected variant of Fragment 5 is coupled with the protected variant of Fragment 6 under classical solution phase peptide coupling conditions. In some embodiments, the protected variant of Fragment 5 is coupled with the protected variant of Fragment 6 under tag-assisted liquid phase peptide coupling conditions.

In other embodiments, the protected variant of Fragment 5 is coupled with the protected variant of Fragment 6 on solid support. In other embodiments, the protected variant of Fragment 5 is coupled with the protected variant of Fragment 6 under solid phase peptide coupling conditions.

In some embodiments, a C-terminal carboxyl group of the protected variant of Fragment 5 is pre-activated. In other embodiments, a C-terminal carboxyl group of the protected variant of Fragment 5 is activated in situ.

In some embodiments, the method comprises:
contacting the protected variant of Fragment 5 with a coupling composition to form a protected variant of Fragment 5 with an activated C-terminal carboxyl group (“activated Fragment 5”); and
contacting the protected variant of Fragment 6 with the activated Fragment 5 to form the protected variant of Peptide B.

In some embodiments, the method comprises:
contacting the protected variant of Fragment 5 with a coupling composition on solid support to form a protected variant of Fragment 5 with an activated C-terminal carboxyl group (“activated Fragment 5”); and
contacting the protected variant of Fragment 6 with the activated Fragment 5 on solid support to form the protected variant of Peptide B.

In some embodiments, the method comprises:
contacting the protected variant of Fragment 5 with a coupling composition in solution to form a protected variant of Fragment 5 with an activated C-terminal carboxyl group (“activated Fragment 5”); and
contacting the protected variant of Fragment 6 with the activated Fragment 5 in solution to form the protected variant of Peptide B.

In some embodiments (e.g., certain embodiments in which a C-terminal carboxyl group of the protected variant of Fragment 5 is pre-activated), the method comprises:
contacting the protected variant of Fragment 5 with a coupling composition to form a protected variant of Fragment 5 with an activated C-terminal carboxyl group (“activated Fragment 5”); and
contacting the protected variant of Fragment 6 with the activated Fragment 5 to form the protected variant of Peptide B, wherein the contacting does not occur in the presence of a coupling reagent.

In some embodiments, the protected variant of Fragment 5 is present in molar excess relative to the coupling reagent in the coupling composition during the contacting of the protected variant of Fragment 5 and the coupling composition.

In some embodiments (e.g., certain embodiments in which a C-terminal carboxyl group of the protected variant of Fragment 5 is pre-activated), the method comprises:
contacting the protected variant of Fragment 5 with a coupling composition on solid support to form a protected variant of Fragment 5 with an activated C-terminal carboxyl group (“activated Fragment 5”); and
contacting the protected variant of Fragment 6 with the activated Fragment 5 on solid support to form the protected variant of Peptide B, wherein the contacting does not occur in the presence of a coupling reagent.

In some embodiments, the protected variant of Fragment 5 is present in molar excess relative to the coupling reagent in the coupling composition during the contacting of the protected variant of Fragment 5 and the coupling composition.

In some embodiments (e.g., certain embodiments in which a C-terminal carboxyl group of the protected variant of Fragment 5 is pre-activated), the method comprises:
contacting the protected variant of Fragment 5 with a coupling composition in solution to form a protected variant of Fragment 5 with an activated C-terminal carboxyl group (“activated Fragment 5”); and
contacting the protected variant of Fragment 6 with the activated Fragment 5 in solution to form the protected variant of Peptide B, wherein the contacting does not occur in the presence of a coupling reagent.

In some embodiments, the protected variant of Fragment 5 is present in molar excess relative to the coupling reagent in the coupling composition during the contacting of the protected variant of Fragment 5 and the coupling composition.

In some embodiments, the contacting of the protected variant of Fragment 5 with the coupling composition and the contacting of the activated Fragment 5 with the protected variant of Fragment 6 occur in the same reaction vessel. In other embodiments, the contacting of the protected variant of Fragment 5 with the coupling composition and the contacting of the activated Fragment 5 with the protected variant of Fragment 6 occur in different reaction vessels.

In some embodiments, the activated Fragment 5 is contacted with the protected variant of Fragment 6 in the presence of a base and/or a coupling additive but not a coupling reagent (such as, e.g., in certain embodiments in which a C-terminal carboxyl group of the protected variant of Fragment 5 is pre-activated and the protected variant of Fragment 5 is present in molar excess relative to the coupling reagent in the coupling composition during the contacting to form activated Fragment 5).

In some embodiments in which a C-terminal carboxyl group of the protected variant of Fragment 5 is pre-activated, the protected variant of Fragment 6 has a free N-terminal amino group and a free C-terminal carboxyl group.

In some embodiments, the coupling composition comprises a coupling reagent and a base. In some embodiments, the coupling composition comprises a coupling reagent (e.g., a carbodiimide, e.g., DIC, DCC, EDC) and a coupling additive (e.g., HOBt, 5-Cl—HOBt, Oxyma).

In some embodiments, the coupling composition comprises a coupling reagent, a coupling additive, and a base.

In some embodiments, the coupling reagent is a carbodiimide, such as, e.g., DIC, DCC, or EDC. In other embodiments, the coupling reagent is BOP—Cl.

In some embodiments (e.g., certain embodiments in which a C-terminal carboxyl group of the protected variant of Fragment 5 is activated in situ), the method comprises coupling the protected variant of Fragment 5 with the protected variant of Fragment 6 in the presence of a coupling composition. In some embodiments, the method comprises coupling the protected variant of Fragment 5 with the protected variant of Fragment 6 in solution in the presence of a coupling composition.

In some embodiments, the protected variant of Fragment 6 does not have a free carboxyl group at the C-terminus. In some embodiments, the C-terminus of the protected variant of Fragment 6 is protected. In some embodiments, the C-terminus of the protected variant of Fragment 6 is amidated. In some embodiments, the protected variant of Fragment 6 has a free amino group at the N-terminus. In preferred embodiments, the protected variant of Fragment 6 has a free amino group at the N-terminus and does not have a free carboxyl group at the C-terminus. In particularly preferred embodiments, the protected variant of Fragment 6 has a free amino group at the N-terminus and an amidated C-terminus.

In some embodiments, the protected variant of Fragment 5 does not have a free amino group at the N-terminus. In some embodiments, the N-terminus of the protected variant of Fragment 5 is protected (preferably Fmoc- or Trt-protected, most preferably Trt-protected). In some embodiments, the protected variant of Fragment 5 has a free carboxyl group at the C-terminus. In preferred embodiments, the protected variant of Fragment 5 has a free carboxyl group at the C-terminus and does not have a free amino group at the N-terminus. In particularly preferred embodiments, the protected variant of Fragment 5 has a free carboxyl group at the C-terminus and a protected (preferably Fmoc- or Trt-protected, most preferably Trt-protected) N-terminus.

In some embodiments, the protected variant of Fragment 6 has a free amino group at the N-terminus and does not have a free carboxyl group at the C-terminus, and the protected variant of Fragment 5 has a free carboxyl group at the C-terminus and a protected (preferably Fmoc- or Trt-protected, most preferably Trt-protected) N-terminus.

In some embodiments, the C-terminus of the protected variant of Fragment 6 is amidated, and the N-terminus of the protected variant of Fragment 5 is protected (preferably Fmoc- or Trt-protected, most preferably Trt-protected).

In preferred embodiments, the protected variant of Fragment 6 has a free amino group at the N-terminus and does not have a free carboxyl group at the C-terminus, and the protected variant of Fragment 5 has a free carboxyl group at the C-terminus and does not have a free amino group at the N-terminus. In particularly preferred embodiments, the protected variant of Fragment 6 has a free amino group at the N-terminus and an amidated C-terminus, and the protected variant of Fragment 5 has a free carboxyl group at the C-terminus and a protected (preferably Fmoc- or Trt-protected, most preferably Trt-protected) N-terminus.

In some embodiments, the protected variant of Fragment 6 has a free amino group at the N-terminus and an amidated C-terminus, and the protected variant of Fragment 5 has a free carboxyl group at the C-terminus and an Fmoc-protected N-terminus. In some embodiments, the protected variant of Fragment 6 has a free amino group at the N-terminus and an amidated C-terminus, and the protected variant of Fragment 5 has a free carboxyl group at the C-terminus and a Trt-protected N-terminus.

In some embodiments, the N-terminus, the side chain of the Trp residue at position 2, and the side chain of the Lys residue at position 5 of the protected variant of Fragment 5 are protected and the protected variant of Fragment 5 has a free carboxyl group at the C-terminus.

In some embodiments, the side chain of the Ser residue at position 5, the side chain of the Ser residue at position 10, the side chain of the Ser residue at position 15, and the side chain of the Lys residue at position 16 of the protected variant of Fragment 6 are protected, the protected variant of Fragment 6 has a free amino group at the N-terminus, and the protected variant of Fragment 6 does not have a free carboxyl group at the C-terminus.

In some embodiments, the N-terminus, the side chain of the Trp residue at position 2, and the side chain of the Lys residue at position 5 of the protected variant of Fragment 5 are protected, the protected variant of Fragment 5 has a free carboxyl group at the C-terminus, the side chain of the Ser residue at position 5, the side chain of the Ser residue at position 10, the side chain of the Ser residue at position 15, and the side chain of the Lys residue at position 16 of the protected variant of Fragment 6 are protected, the protected variant of Fragment 6 has a free amino group at the N-terminus, and the protected variant of Fragment 6 does not have a free carboxyl group at the C-terminus.

In some embodiments, the side chain of the Trp residue at position 2 and the side chain of the Lys residue at position 5 of the protected variant of Fragment 5 are each protected with a tert-butyloxycarbonyl (Boc) protecting group.

In some embodiments, the Ser residues at position 5, position 10, and position 15 of the protected variant of Fragment 6 are each protected as a dimethylated pseudoproline (Psi(Me,Me) pro) moiety in which the oxazolidine is derived from Ser and the side chain of the Lys residue at position 16 of the protected variant of Fragment 6 is protected with an ivDde (1-(4,4-dimethyl-2,6-dioxocyclohex-1-ylidene)-3-methylbutyl) protecting group.

In some embodiments, the side chain of the Trp residue at position 2 and the side chain of the Lys residue at position 5 of the protected variant of Fragment 5 are each protected with a tert-butyloxycarbonyl (Boc) protecting group, the Ser residues at position 5, position 10, and position 15 of the protected variant of Fragment 6 are each protected as a dimethylated pseudoproline (Psi(Me,Me) pro) moiety in which the oxazolidine is derived from Ser, and the side chain of the Lys residue at position 16 of the protected variant of Fragment 6 is protected with an ivDde (1-(4,4-dimethyl-2,6-dioxocyclohex-1-ylidene)-3-methylbutyl) protecting group.

In some embodiments, the protected variant of Fragment 5 is Trt-Ala-Trp(Boc)-Leu-Val-Lys(Boc)-Gly-Gly-Gly-OH (SEQ ID NO: 59).

In some embodiments, the protected variant of Fragment 6 is Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130) or Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 121). In some embodiments, the protected variant of Fragment 6 is Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130). In some embodiments, the protected variant of Fragment 6 is Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 121).

In some embodiments, the protected variant of Fragment 5 is Trt-Ala-Trp(Boc)-Leu-Val-Lys(Boc)-Gly-Gly-Gly-OH (SEQ ID NO: 59), and the protected variant of Fragment 6 is Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130) or Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 121).

In some embodiments, the protected variant of Fragment 5 is Trt-Ala-Trp(Boc)-Leu-Val-Lys(Boc)-Gly-Gly-Gly-OH (SEQ ID NO: 59), and the protected variant of Fragment 6 is Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130).

In some embodiments, the protected variant of Fragment 5 is Trt-Ala-Trp(Boc)-Leu-Val-Lys(Boc)-Gly-Gly-Gly-OH (SEQ ID NO: 59), and the protected variant of Fragment 6 is Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 121).

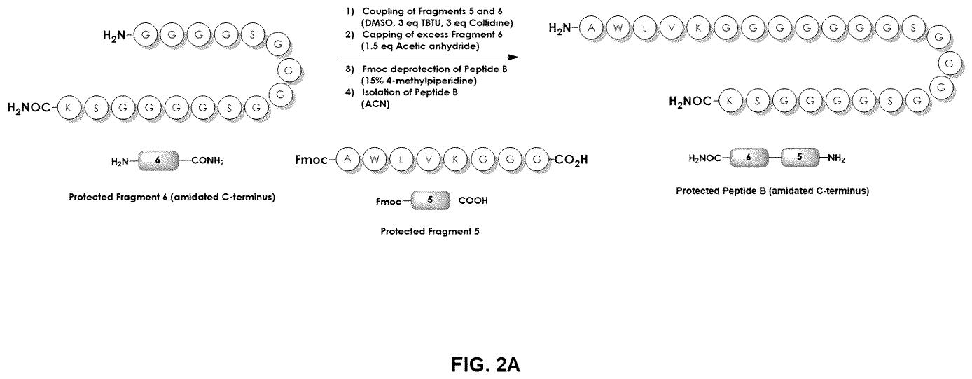

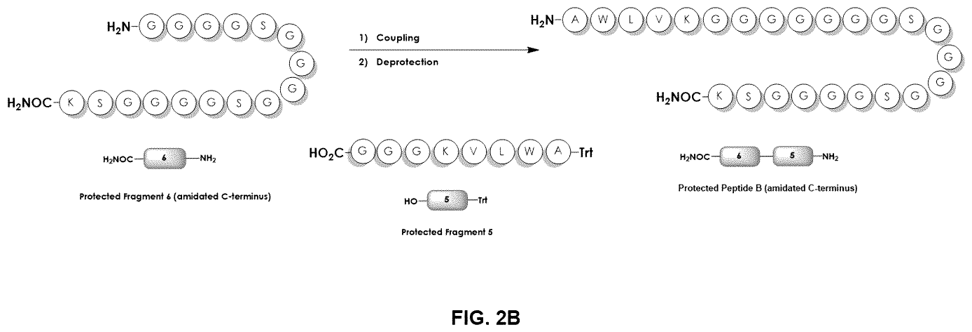

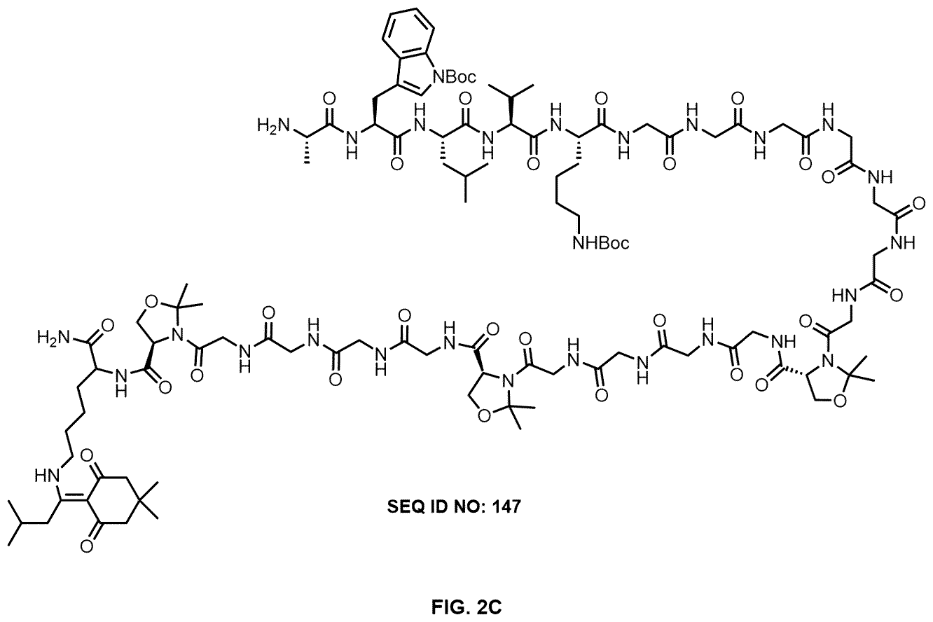

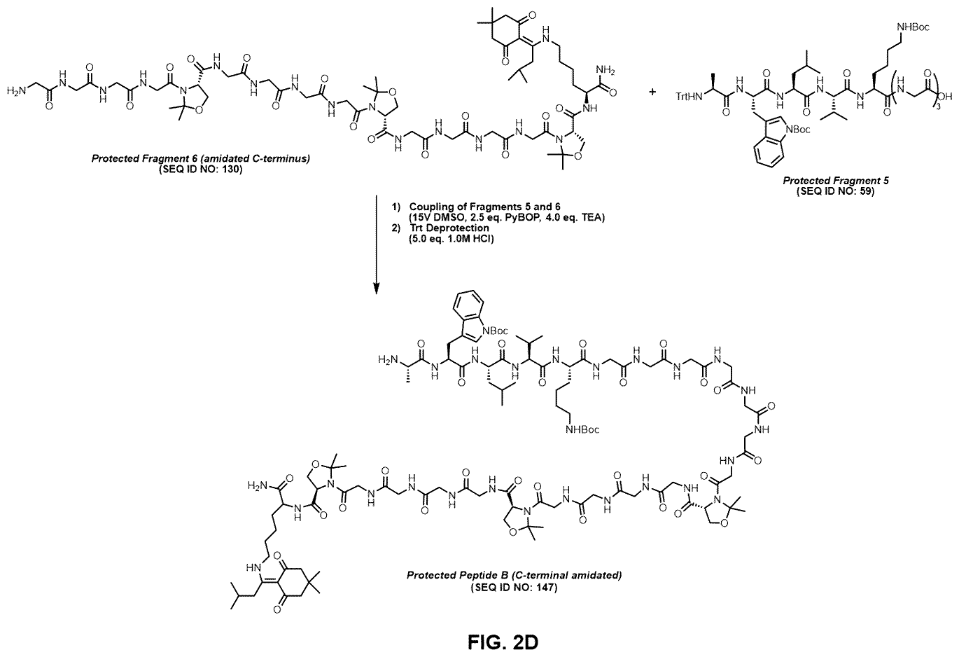

In some embodiments, a protected variant of Peptide B can be efficiently prepared in solution by coupling a protected variant of Fragment 6 with an amidated C-terminus and a protected variant of Fragment 5 to form a protected variant of Peptide B with an amidated C-terminus as shown in FIG. 2A. Alternatively, a protected variant of Peptide B can be efficiently prepared in solution by coupling a protected variant of Fragment 6 with an amidated C-terminus and another protected variant of Fragment 5 to form a protected variant of Peptide B with an amidated C-terminus as shown in FIG. 2B. FIG. 2C depicts another protected variant of Peptide B with an amidated C-terminus, which can be prepared by an analogous process to that shown in FIG. 2B. Alternatively, a protected variant of Peptide B can be efficiently prepared in solution by coupling a protected variant of Fragment 6 with an amidated C-terminus and another protected variant of Fragment 5 to form a protected variant of Peptide B with an amidated C-terminus as shown in FIG. 2D.

In some embodiments, the coupling composition comprises 1 to 10 equivalents of a coupling reagent, optionally 1 to 10 equivalents of a base, and optionally 1 to 10 equivalents of a coupling additive, wherein equivalents are relative to the protected variant of Fragment 5. In some embodiments, the coupling composition comprises 2 to 10 equivalents of a coupling reagent, optionally 2 to 10 equivalents of a base, and optionally 2 to 10 equivalents of a coupling additive, wherein equivalents are relative to the protected variant of Fragment 5.

In some embodiments, the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the coupling composition comprises PyBOP and TEA. In some embodiments, the coupling composition comprises PyBOP and TEA in DMSO. In some embodiments, the coupling composition comprises PyBOP and TEA in DMSO and MeOH. In some embodiments, the coupling composition comprises PyBOP and TEA in 1-5:1 DMSO:MeOH. In some embodiments, the coupling composition comprises PyBOP and TEA in 1:1 DMSO:MeOH.

In some embodiments, the coupling composition comprises TBTU and collidine. In some embodiments, the coupling composition comprises TBTU and collidine in a solvent selected from DMSO, acetonitrile, and combinations thereof. In some embodiments, the coupling composition comprises TBTU and collidine in DMSO. In some embodiments, the coupling composition comprises TBTU and collidine in acetonitrile. In some embodiments, the coupling composition comprises TBTU and collidine in DMSO and acetonitrile.

In some embodiments, the coupling composition comprises TBTU and TMEDA. In some embodiments, the coupling composition comprises TBTU and TMEDA in acetonitrile.

In some embodiments, the protected variant of Fragment 5 is Trt-Ala-Trp(Boc)-Leu-Val-Lys(Boc)-Gly-Gly-Gly-OH (SEQ ID NO: 59), the protected variant of Fragment 6 is Gly-Gly-Gly-Gly-Ser(Psi(Me, Me)Pro)-Gly-Gly-Gly-Gly-Ser(Psi(Me, Me)Pro)-Gly-Gly-Gly-Gly-Ser(Psi(Me,Me)Pro)-Lys(ivDde)-NH₂ (SEQ ID NO: 130) or Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 121), and the coupling composition comprises TBTU/collidine, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the protected variant of Fragment 5 is Trt-Ala-Trp(Boc)-Leu-Val-Lys(Boc)-Gly-Gly-Gly-OH (SEQ ID NO: 59), the protected variant of Fragment 6 is Gly-Gly-Gly-Gly-Ser(Psi(Me, Me)Pro)-Gly-Gly-Gly-Gly-Ser(Psi(Me, Me)Pro)-Gly-Gly-Gly-Gly-Ser(Psi(Me,Me)Pro)-Lys(ivDde)-NH₂ (SEQ ID NO: 130), and the coupling composition comprises PyBOP and TEA. In some embodiments, the coupling composition comprises PyBOP and TEA in DMSO. In some embodiments, the coupling composition comprises PyBOP and TEA in 1-5:1 DMSO:MeOH. In some embodiments, the coupling composition comprises PyBOP and TEA in 1:1 DMSO:MeOH.

In some embodiments, the protected variant of Fragment 5 is Trt-Ala-Trp(Boc)-Leu-Val-Lys(Boc)-Gly-Gly-Gly-OH (SEQ ID NO: 59), the protected variant of Fragment 6 is Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130), and the coupling composition comprises TBTU and collidine. In some embodiments, the coupling composition comprises TBTU and collidine in a solvent selected from DMSO, acetonitrile, and combinations thereof. In some embodiments, the protected variant of Fragment 5 is Trt-Ala-Trp(Boc)-Leu-Val-Lys(Boc)-Gly-Gly-Gly-OH (SEQ ID NO: 59), the protected variant of Fragment 6 is Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130), and the coupling composition comprises TBTU and TMEDA. In some embodiments, the coupling composition comprises TBTU and TMEDA in acetonitrile.

In some embodiments, the protected variant of Fragment 5 is Trt-Ala-Trp(Boc)-Leu-Val-Lys(Boc)-Gly-Gly-Gly-OH (SEQ ID NO: 59), the protected variant of Fragment 6 is Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 121), and the coupling composition comprises TBTU and collidine. In some embodiments, the coupling composition comprises TBTU and collidine in DMSO and acetonitrile.

In some embodiments, the coupling occurs at a temperature in the range of 0° C. to 60° C. In some embodiments, the coupling occurs at a temperature in the range of 20° C. to 50° C. In some embodiments, the coupling occurs at a temperature in the range of 20° C. to 30° C. In some embodiments, the coupling occurs at a temperature in the range of 30° C. to 50° C. In some embodiments, the coupling occurs at a temperature in the range of 35° C. to 45° C.

In some embodiments, the method comprises coupling the protected variant of Fragment 5 and the protected variant of Fragment 6 for a time in the range of 1 hour to 72 hours. In some embodiments, the time is in the range of 6 hours to 24 hours. In other embodiments, the time is in the range of 1 hour to 2 hours.

In some embodiments, the coupling occurs at a temperature in the range of 0° C. to 60° C. for a time in the range of 1 hour to 72 hours. In some embodiments, the coupling occurs at a temperature in the range of 0° C. to 60° C. for a time in the range of 6 hours to 24 hours. In some embodiments, the coupling occurs at a temperature in the range of 20° C. to 50° C. for a time in the range of 1 hour to 72 hours. In some embodiments, the coupling occurs at a temperature in the range of 20° C. to 50° C. for a time in the range of 6 hours to 24 hours.

In some embodiments, the coupling occurs at a temperature in the range of 20° C. to 40° C. for a time in the range of 1 hour to 2 hours.

In some embodiments, the protected variant of Fragment 5 is Trt-Ala-Trp(Boc)-Leu-Val-Lys(Boc)-Gly-Gly-Gly-OH (SEQ ID NO: 59), the protected variant of Fragment 6 is Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me, Me)Pro)-Lys(ivDde)-NH₂ (SEQ ID NO: 130) or Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 121), the coupling composition comprises TBTU/collidine, PyBOP/TEA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine (preferably TBTU/collidine or PyBOP/TEA), and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours).

In some embodiments, the method further comprises selectively deprotecting the N-terminus of the protected variant of Peptide B (e.g., using a Trt, Boc, or Fmoc deprotection method described herein).

In some embodiments, the N-terminus of the protected variant of Peptide B formed by coupling the protected variant of Fragment 5 with the protected variant of Fragment 6 is Trt-protected, and the method further comprises contacting the protected variant of Peptide B with a Brønsted acid or a Lewis acid (preferably TFA or HCl), optionally in the presence of a scavenger (preferably TIS), to selectively remove the trityl protecting group at the N-terminus. In some embodiments, the protected variant of Peptide B is contacted with a Brønsted acid (preferably a dilute Brønsted acid). In some embodiments, the Brønsted acid is selected from TFA, HCl, and dichloroacetic acid (preferably dilute TFA, HCl, or dichloroacetic acid). In other embodiments, the protected variant of Peptide B is contacted with a Lewis acid (preferably a dilute Lewis acid).

In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing.

In some embodiments, the method further comprises contacting the protected variant of Peptide B with HCl. In some embodiments, the method further comprises contacting the protected variant of Peptide B with dilute HCl. In some embodiments, the method further comprises contacting the protected variant of Peptide B with 1 eq. to 10 eq. HCl. In some embodiments, the method further comprises contacting the protected variant of Peptide B with 1 eq. to 5 eq. HCl. In some embodiments, the method further comprises contacting the protected variant of Peptide B with 2 eq. to 5 eq. HCl.

In some embodiments, the method further comprises contacting the protected variant of Peptide B with TFA in the presence of TIS. In some embodiments, the method further comprises contacting the protected variant of Peptide B with dilute TFA in the presence of TIS.

In some embodiments, the contacting occurs at a temperature in the range of 0° C. to 60° C. for a time in the range of 1 hour to 72 hours.

In some embodiments, the N-terminus of the protected variant of Peptide B formed by coupling the protected variant of Fragment 5 with the protected variant of Fragment 6 is Trt-protected, and the method further comprises contacting the protected variant of Peptide B with a Brønsted acid or a Lewis acid (preferably TFA or HCl), optionally in the presence of a scavenger selected from hydrosilanes, phenols, thioethers, thiols/dithiols, water, indole, and combinations of any of the foregoing (preferably TIS), to selectively remove the trityl protecting group at the N-terminus, wherein the contacting occurs at a temperature in the range of 0° C. to 60° C. for a time in the range of 1 hour to 72 hours. In some embodiments, the contacting occurs at a temperature in the range of 30° C. to 50° C. for a time in the range of 6 hour to 24 hours.

In some embodiments, the N-terminus of the protected variant of Peptide B formed by coupling the protected variant of Fragment 5 with the protected variant of Fragment 6 is Trt-protected, and the method further comprises contacting the protected variant of Peptide B with a dilute Brønsted acid or a dilute Lewis acid (preferably dilute TFA or HCl), optionally in the presence of a scavenger selected from hydrosilanes, phenols, thioethers, thiols/dithiols, water, indole, and combinations of any of the foregoing (preferably TIS), to selectively remove the trityl protecting group at the N-terminus, wherein the contacting occurs at a temperature in the range of 0° C. to 60° C. for a time in the range of 1 hour to 72 hours. In some embodiments, the contacting occurs at a temperature in the range of 30° C. to 50° C. for a time in the range of 6 hour to 24 hours.

In some embodiments, the N-terminus of the protected variant of Peptide B formed by coupling the protected variant of Fragment 5 with the protected variant of Fragment 6 is Trt-protected, and the method further comprises contacting the protected variant of Peptide B with 1 to 10 eq. HCl to selectively remove the trityl protecting group at the N-terminus, wherein the contacting occurs at a temperature in the range of 25° C. to 50° C. (e.g., 25° C. to 35° C., e.g., 30° C.) for a time in the range of 6 hour to 36 hours (e.g., 12 hours to 24 hours). In some embodiments, the N-terminus of the protected variant of Peptide B formed by coupling the protected variant of Fragment 5 with the protected variant of Fragment 6 is Trt-protected, and the method further comprises contacting the protected variant of Peptide B with 2 eq. to 5 eq. HCl to selectively remove the trityl protecting group at the N-terminus, wherein the contacting occurs at a temperature in the range of 25° C. to 35° C. for a time in the range of 12 hours to 24 hours.

Also provided herein is a method of preparing Peptide B or a protected variant thereof, comprising coupling a protected variant of Fragment 3A with a protected variant of Fragment 4 to form a protected variant of Peptide B, wherein Peptide B comprises the amino acid sequence of SEQ ID NO: 9, Fragment 3A comprises the amino acid sequence of SEQ ID NO: 13, and Fragment 4 comprises the amino acid sequence of SEQ ID NO: 4.

In some embodiments, the protected variant of Fragment 3A is coupled with the protected variant of Fragment 4 in solution. In some embodiments, the protected variant of Fragment 3A is coupled with the protected variant of Fragment 4 under liquid phase peptide coupling conditions. In some embodiments, the protected variant of Fragment 3A is coupled with the protected variant of Fragment 4 under classical solution phase peptide coupling conditions. In some embodiments, the protected variant of Fragment 3A is coupled with the protected variant of Fragment 4 under tag-assisted liquid phase peptide coupling conditions.

In other embodiments, the protected variant of Fragment 3A is coupled with the protected variant of Fragment 4 on solid support. In some embodiments, the protected variant of Fragment 3A is coupled with the protected variant of Fragment 4 under solid phase peptide coupling conditions.

In some embodiments, a C-terminal carboxyl group of the protected variant of Fragment 3A is pre-activated. In other embodiments, a C-terminal carboxyl group of the protected variant of Fragment 3A is activated in situ.

In some embodiments, the method comprises:
contacting the protected variant of Fragment 3A with a coupling composition to form a protected variant of Fragment 3A with an activated C-terminal carboxyl group (“activated Fragment 3A”); and
contacting the protected variant of Fragment 4 with the activated Fragment 3A to form the protected variant of Peptide B.

In some embodiments, the method comprises:
contacting the protected variant of Fragment 5 with a coupling composition on solid support to form a protected variant of Fragment 5 with an activated C-terminal carboxyl group (“activated Fragment 5”); and
contacting the protected variant of Fragment 6 with the activated Fragment 5 on solid support to form the protected variant of Peptide B.

In some embodiments, the method comprises:
contacting the protected variant of Fragment 3A with a coupling composition in solution to form a protected variant of Fragment 3A with an activated C-terminal carboxyl group (“activated Fragment 3A”); and
contacting the protected variant of Fragment 4 with the activated Fragment 3A in solution to form the protected variant of Peptide B.

In some embodiments (e.g., certain embodiments in which a C-terminal carboxyl group of the protected variant of Fragment 3A is pre-activated), the method comprises:
contacting the protected variant of Fragment 3A with a coupling composition to form a protected variant of Fragment 3A with an activated C-terminal carboxyl group (“activated Fragment 3A”); and
contacting the protected variant of Fragment 4 with the activated Fragment 3A to form the protected variant of Peptide B, wherein the contacting does not occur in the presence of a coupling reagent.

In some embodiments, the protected variant of Fragment 3A is present in molar excess relative to the coupling reagent in the coupling composition during the contacting of the protected variant of Fragment 3A and the coupling composition.

In some embodiments (e.g., certain embodiments in which a C-terminal carboxyl group of the protected variant of Fragment 3A is pre-activated), the method comprises:
contacting the protected variant of Fragment 3A with a coupling composition on solid support to form a protected variant of Fragment 3A with an activated C-terminal carboxyl group (“activated Fragment 3A”); and
contacting the protected variant of Fragment 4 with the activated Fragment 3A on solid support to form the protected variant of Peptide B, wherein the contacting does not occur in the presence of a coupling reagent.

In some embodiments, the protected variant of Fragment 3A is present in molar excess relative to the coupling reagent in the coupling composition during the contacting of the protected variant of Fragment 3A and the coupling composition.

In some embodiments (e.g., certain embodiments in which a C-terminal carboxyl group of the protected variant of Fragment 3A is pre-activated), the method comprises:
contacting the protected variant of Fragment 3A with a coupling composition in solution to form a protected variant of Fragment 3A with an activated C-terminal carboxyl group (“activated Fragment 3A”); and
contacting the protected variant of Fragment 4 with the activated Fragment 3A in solution to form the protected variant of Peptide B, wherein the contacting does not occur in the presence of a coupling reagent.

In some embodiments, the protected variant of Fragment 3A is present in molar excess relative to the coupling reagent in the coupling composition during the contacting of the protected variant of Fragment 3A and the coupling composition.

In some embodiments, the contacting of the protected variant of Fragment 3A with the coupling composition and the contacting of the activated Fragment 3A with the protected variant of Fragment 4 occur in the same reaction vessel. In other embodiments, the contacting of the protected variant of Fragment 3A with the coupling composition and the contacting of the activated Fragment 3A with the protected variant of Fragment 4 occur in different reaction vessels.

In some embodiments, the activated Fragment 3A is contacted with the protected variant of Fragment 4 in the presence of a base and/or a coupling additive but not a coupling reagent (such as, e.g., in certain embodiments in which a C-terminal carboxyl group of the protected variant of Fragment 3A is pre-activated and the protected variant of Fragment 3A is present in molar excess relative to the coupling reagent in the coupling composition during the contacting to form activated Fragment 3A).

In some embodiments in which a C-terminal carboxyl group of the protected variant of Fragment 3A is pre-activated, the protected variant of Fragment 4 has a free N-terminal amino group and a free C-terminal carboxyl group.

In some embodiments, the coupling composition comprises a coupling reagent and a base. In some embodiments, the coupling composition comprises a coupling reagent (e.g., a carbodiimide, e.g., DIC, DCC, EDC) and a coupling additive (e.g., HOBt, 5-Cl—HOBt, Oxyma).

In some embodiments, the coupling composition comprises a coupling reagent, a coupling additive, and a base.

In some embodiments, the coupling reagent is a carbodiimide, such as, e.g., DIC, DCC, or EDC. In other embodiments, the coupling reagent is BOP—Cl.

In some embodiments (e.g., certain embodiments in which a C-terminal carboxyl group of the protected variant of Fragment 3A is activated in situ), the method comprises coupling the protected variant of Fragment 3A with the protected variant of Fragment 4 in the presence of a coupling composition. In some embodiments, the method comprises coupling the protected variant of Fragment 3A with the protected variant of Fragment 4 in solution in the presence of a coupling composition.

In some embodiments, the protected variant of Fragment 4 does not have a free carboxyl group at the C-terminus. In some embodiments, the C-terminus of the protected variant of Fragment 4 is protected. In some embodiments, the C-terminus of the protected variant of Fragment 4 is amidated. In some embodiments, the protected variant of Fragment 4 has a free amino group at the N-terminus. In preferred embodiments, the protected variant of Fragment 4 has a free amino group at the N-terminus and does not have a free carboxyl group at the C-terminus. In particularly preferred embodiments, the protected variant of Fragment 4 has a free amino group at the N-terminus and an amidated C-terminus.

In some embodiments, the protected variant of Fragment 3A does not have a free amino group at the N-terminus. In some embodiments, the N-terminus of the protected variant of Fragment 3A is protected (preferably Fmoc- or Trt-protected, most preferably Trt-protected). In some embodiments, the protected variant of Fragment 3A has a free carboxyl group at the C-terminus. In preferred embodiments, the protected variant of Fragment 3A has a free carboxyl group at the C-terminus and does not have a free amino group at the N-terminus. In particularly preferred embodiments, the protected variant of Fragment 3A has a free carboxyl group at the C-terminus and a protected (preferably Fmoc- or Trt-protected, most preferably Trt-protected) N-terminus.

In some embodiments, the protected variant of Fragment 4 has a free amino group at the N-terminus and does not have a free carboxyl group at the C-terminus, and the protected variant of Fragment 3A has a free carboxyl group at the C-terminus and a protected (preferably Fmoc- or Trt-protected, most preferably Trt-protected) N-terminus.

In some embodiments, the C-terminus of the protected variant of Fragment 4 is amidated, and the N-terminus of the protected variant of Fragment 3A is protected (preferably Fmoc- or Trt-protected, most preferably Trt-protected).

In preferred embodiments, the protected variant of Fragment 4 has a free amino group at the N-terminus and does not have a free carboxyl group at the C-terminus, and the protected variant of Fragment 3A has a free carboxyl group at the C-terminus and does not have a free amino group at the N-terminus. In particularly preferred embodiments, the protected variant of Fragment 4 has a free amino group at the N-terminus and an amidated C-terminus, and the protected variant of Fragment 3A has a free carboxyl group at the C-terminus and a protected (preferably Fmoc- or Trt-protected, most preferably Trt-protected) N-terminus.

In some embodiments, the protected variant of Fragment 4 has a free amino group at the N-terminus and an amidated C-terminus, and the protected variant of Fragment 3A has a free carboxyl group at the C-terminus and an Fmoc-protected N-terminus. In some embodiments, the protected variant of Fragment 4 has a free amino group at the N-terminus and an amidated C-terminus, and the protected variant of Fragment 3A has a free carboxyl group at the C-terminus and a Trt-protected N-terminus.

In some embodiments, the protected variant of Fragment 3A is Fmoc-Ala-Trp(Boc)-Leu-Val-Lys(Boc)-Gly7-OH (SEQ ID NO: 57).

In some embodiments, the protected variant of Fragment 4 is Ser(tBu)-Gly4-Ser(tBu)-Gly4-Ser(tBu)-Lys(ivDde)-NH₂ (SEQ ID NO: 109).

In some embodiments, the protected variant of Fragment 3A is Fmoc-Ala-Trp(Boc)-Leu-Val-Lys(Boc)-Gly7-OH (SEQ ID NO: 57), and the protected variant of Fragment 4 is Ser(tBu)-Gly4-Ser(tBu)-Gly4-Ser(tBu)-Lys(ivDde)-NH₂ (SEQ ID NO: 109).

In some embodiments, the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the coupling composition comprises DIC/Oxyma, PyBOP/collidine, PyOxim/collidine, or TBTU/collidine. In some embodiments, the coupling composition comprises DIC/Oxyma, PyBOP/collidine, PyOxim/collidine, or TBTU/collidine in DSMO.

In some embodiments, the coupling composition comprises DIC and Oxyma. In some embodiments, the coupling composition comprises DIC and Oxyma in DMSO.

In some embodiments, the coupling composition comprises TBTU and collidine. In some embodiments, the coupling composition comprises TBTU and collidine in DMSO.

In some embodiments, the protected variant of Fragment 3A is Fmoc-Ala-Trp(Boc)-Leu-Val-Lys(Boc)-Gly7-OH (SEQ ID NO: 57), the protected variant of Fragment 4 is Ser(tBu)-Gly4-Ser(tBu)-Gly4-Ser(tBu)-Lys(ivDde)-NH₂ (SEQ ID NO: 109), and the coupling composition comprises TBTU/collidine, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the protected variant of Fragment 3A is Fmoc-Ala-Trp(Boc)-Leu-Val-Lys(Boc)-Gly7-OH (SEQ ID NO: 57), the protected variant of Fragment 4 is Ser(tBu)-Gly4-Ser(tBu)-Gly4-Ser(tBu)-Lys(ivDde)-NH₂ (SEQ ID NO: 109), and the coupling composition comprises DIC/Oxyma, PyBOP/collidine, PyOxim/collidine, or TBTU/collidine. In some embodiments, the coupling composition comprises DIC/Oxyma, PyBOP/collidine, PyOxim/collidine, or TBTU/collidine in DMSO.

In some embodiments, the protected variant of Fragment 3A is Fmoc-Ala-Trp(Boc)-Leu-Val-Lys(Boc)-Gly7-OH (SEQ ID NO: 57), the protected variant of Fragment 4 is Ser(tBu)-Gly4-Ser(tBu)-Gly4-Ser(tBu)-Lys(ivDde)-NH₂ (SEQ ID NO: 109), and the coupling composition comprises DIC and Oxyma. In some embodiments, the coupling composition comprises DIC and Oxyma in DMSO.

In some embodiments, the protected variant of Fragment 3A is Fmoc-Ala-Trp(Boc)-Leu-Val-Lys(Boc)-Gly7-OH (SEQ ID NO: 57), the protected variant of Fragment 4 is Ser(tBu)-Gly4-Ser(tBu)-Gly4-Ser(tBu)-Lys(ivDde)-NH₂ (SEQ ID NO: 109), and the coupling composition comprises TBTU and collidine. In some embodiments, the coupling composition comprises TBTU and collidine in DMSO.

In some embodiments, the coupling occurs at a temperature in the range of 0° C. to 60° C. In some embodiments, the coupling occurs at a temperature in the range of 20° C. to 50° C. In some embodiments, the coupling occurs at a temperature in the range of 20° C. to 30° C. In some embodiments, the coupling occurs at a temperature in the range of 30° C. to 50° C. In some embodiments, the coupling occurs at a temperature in the range of 35° C. to 45° C.

In some embodiments, the method comprises coupling the protected variant of Fragment 3A and the protected variant of Fragment 4 for a time in the range of 1 hour to 72 hours. In some embodiments, the time is in the range of 6 hours to 24 hours.

In some embodiments, the coupling occurs at a temperature in the range of 0° C. to 60° C. for a time in the range of 1 hour to 72 hours. In some embodiments, the coupling occurs at a temperature in the range of 0° C. to 60° C. for a time in the range of 6 hours to 24 hours. In some embodiments, the coupling occurs at a temperature in the range of 20° C. to 50° C. for a time in the range of 1 hour to 72 hours. In some embodiments, the coupling occurs at a temperature in the range of 20° C. to 50° C. for a time in the range of 6 hours to 24 hours.

In some embodiments, the protected variant of Fragment 3A is Fmoc-Ala-Trp(Boc)-Leu-Val-Lys(Boc)-Gly7-OH (SEQ ID NO: 57), the protected variant of Fragment 4 is Ser(tBu)-Gly4-Ser(tBu)-Gly4-Ser(tBu)-Lys(ivDde)-NH₂ (SEQ ID NO: 109), the coupling composition comprises TBTU/collidine, PyBOP/TEA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine (preferably TBTU/collidine or DIC/Oxyma, most preferably DIC/Oxyma), and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours).

In some embodiments, the protected variant of Fragment 3A is Fmoc-Ala-Trp(Boc)-Leu-Val-Lys(Boc)-Gly7-OH (SEQ ID NO: 57), the protected variant of Fragment 4 is Ser(tBu)-Gly4-Ser(tBu)-Gly4-Ser(tBu)-Lys(ivDde)-NH₂ (SEQ ID NO: 109), the coupling composition comprises DIC/Oxyma, PyBOP/collidine, PyOxim/collidine, or TBTU/collidine (preferably TBTU/collidine or DIC/Oxyma, most preferably DIC/Oxyma), and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours).

In some embodiments, the method further comprises selectively deprotecting the N-terminus of the protected variant of Peptide B (e.g., using a Trt, Boc, or Fmoc deprotection method described herein).

Also provided herein is a method of preparing Peptide E or a protected variant thereof, comprising coupling a protected variant of Fragment 3 with a protected variant of Fragment 4 to form a protected variant of Peptide E, wherein Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8.

In some embodiments, the protected variant of Fragment 3 is coupled with the protected variant of Fragment 4 in solution. In some embodiments, the protected variant of Fragment 3 is coupled with the protected variant of Fragment 4 under liquid phase peptide coupling conditions. In some embodiments, the protected variant of Fragment 3 is coupled with the protected variant of Fragment 4 under classical solution phase peptide coupling conditions. In some embodiments, the protected variant of Fragment 3 is coupled with the protected variant of Fragment 4 under tag-assisted liquid phase peptide coupling conditions.

In other embodiments, the protected variant of Fragment 3 is coupled with the protected variant of Fragment 4 on solid support. In some embodiments, the protected variant of Fragment 3 is coupled with the protected variant of Fragment 4 under solid phase peptide coupling conditions.

In some embodiments, a C-terminal carboxyl group of the protected variant of Fragment 3 is pre-activated. In other embodiments, a C-terminal carboxyl group of the protected variant of Fragment 3 is activated in situ.

In some embodiments, the method comprises:
contacting the protected variant of Fragment 3 with a coupling composition to form a protected variant of Fragment 3 with an activated C-terminal carboxyl group (“activated Fragment 3”); and
contacting the protected variant of Fragment 4 with the activated Fragment 3 to form the protected variant of Peptide E.

In some embodiments, the method comprises:
contacting the protected variant of Fragment 3 with a coupling composition on solid support to form a protected variant of Fragment 3 with an activated C-terminal carboxyl group (“activated Fragment 3”); and
contacting the protected variant of Fragment 4 with the activated Fragment 3 on solid support to form the protected variant of Peptide E.

In some embodiments, the method comprises:
contacting the protected variant of Fragment 3 with a coupling composition in solution to form a protected variant of Fragment 3 with an activated C-terminal carboxyl group (“activated Fragment 3”); and
contacting the protected variant of Fragment 4 with the activated Fragment 3 in solution to form the protected variant of Peptide E.

In some embodiments (e.g., certain embodiments in which a C-terminal carboxyl group of the protected variant of Fragment 3 is pre-activated), the method comprises:
contacting the protected variant of Fragment 3 with a coupling composition to form a protected variant of Fragment 3 with an activated C-terminal carboxyl group (“activated Fragment 3”); and
contacting the protected variant of Fragment 4 with the activated Fragment 3 to form the protected variant of Peptide E, wherein the contacting does not occur in the presence of a coupling reagent.

In some embodiments, the protected variant of Fragment 3 is present in molar excess relative to the coupling reagent in the coupling composition during the contacting of the protected variant of Fragment 3 and the coupling composition.

In some embodiments (e.g., certain embodiments in which a C-terminal carboxyl group of the protected variant of Fragment 3 is pre-activated), the method comprises:
contacting the protected variant of Fragment 3 with a coupling composition on solid support to form a protected variant of Fragment 3 with an activated C-terminal carboxyl group (“activated Fragment 3”); and
contacting the protected variant of Fragment 4 with the activated Fragment 3 on solid support to form the protected variant of Peptide E, wherein the contacting does not occur in the presence of a coupling reagent.

In some embodiments, the protected variant of Fragment 3 is present in molar excess relative to the coupling reagent in the coupling composition during the contacting of the protected variant of Fragment 3 and the coupling composition.

In some embodiments (e.g., certain embodiments in which a C-terminal carboxyl group of the protected variant of Fragment 3 is pre-activated), the method comprises:
contacting the protected variant of Fragment 3 with a coupling composition in solution to form a protected variant of Fragment 3 with an activated C-terminal carboxyl group (“activated Fragment 3”); and
contacting the protected variant of Fragment 4 with the activated Fragment 3 in solution to form the protected variant of Peptide E, wherein the contacting does not occur in the presence of a coupling reagent.

In some embodiments, the protected variant of Fragment 3 is present in molar excess relative to the coupling reagent in the coupling composition during the contacting of the protected variant of Fragment 3 and the coupling composition.

In some embodiments, the contacting of the protected variant of Fragment 3 with the coupling composition and the contacting of the activated Fragment 3 with the protected variant of Fragment 4 occur in the same reaction vessel. In other embodiments, the contacting of the protected variant of Fragment 3 with the coupling composition and the contacting of the activated Fragment 3 with the protected variant of Fragment 4 occur in different reaction vessels.

In some embodiments, the activated Fragment 3 is contacted with the protected variant of Fragment 4 in the presence of a base and/or a coupling additive but not a coupling reagent (such as, e.g., in certain embodiments in which a C-terminal carboxyl group of the protected variant of Fragment 3 is pre-activated and the protected variant of Fragment 3 is present in molar excess relative to the coupling reagent in the coupling composition during the contacting to form activated Fragment 3).

In some embodiments in which a C-terminal carboxyl group of the protected variant of Fragment 3 is pre-activated, the protected variant of Fragment 4 has a free N-terminal amino group and a free C-terminal carboxyl group.

In some embodiments, the coupling composition comprises a coupling reagent and a base. In some embodiments, the coupling composition comprises a coupling reagent (e.g., a carbodiimide, e.g., DIC, DCC, EDC) and a coupling additive (e.g., HOBt, 5-Cl—HOBt, Oxyma).

In some embodiments, the coupling composition comprises a coupling reagent, a coupling additive, and a base.

In some embodiments, the coupling reagent is a carbodiimide, such as, e.g., DIC, DCC, or EDC. In other embodiments, the coupling reagent is BOP—Cl.

In some embodiments (e.g., certain embodiments in which a C-terminal carboxyl group of the protected variant of Fragment 3 is activated in situ), the method comprises coupling the protected variant of Fragment 3 with the protected variant of Fragment 4 in the presence of a coupling composition. In some embodiments, the method comprises coupling the protected variant of Fragment 3 with the protected variant of Fragment 4 in solution in the presence of a coupling composition.

In some embodiments, the protected variant of Fragment 4 does not have a free carboxyl group at the C-terminus. In some embodiments, the C-terminus of the protected variant of Fragment 4 is protected. In some embodiments, the C-terminus of the protected variant of Fragment 4 is amidated. In some embodiments, the protected variant of Fragment 4 has a free amino group at the N-terminus. In preferred embodiments, the protected variant of Fragment 4 has a free amino group at the N-terminus and does not have a free carboxyl group at the C-terminus. In particularly preferred embodiments, the protected variant of Fragment 4 has a free amino group at the N-terminus and an amidated C-terminus.

In some embodiments, the protected variant of Fragment 3 does not have a free amino group at the N-terminus. In some embodiments, the N-terminus of the protected variant of Fragment 3 is protected (preferably Fmoc- or Trt-protected, most preferably Trt-protected). In some embodiments, the protected variant of Fragment 3 has a free carboxyl group at the C-terminus. In preferred embodiments, the protected variant of Fragment 3 has a free carboxyl group at the C-terminus and does not have a free amino group at the N-terminus. In particularly preferred embodiments, the protected variant of Fragment 3 has a free carboxyl group at the C-terminus and a protected (preferably Fmoc- or Trt-protected, most preferably Trt-protected) N-terminus.

In some embodiments, the protected variant of Fragment 4 has a free amino group at the N-terminus and does not have a free carboxyl group at the C-terminus, and the protected variant of Fragment 3 has a free carboxyl group at the C-terminus and a protected (preferably Fmoc- or Trt-protected, most preferably Trt-protected) N-terminus.

In some embodiments, the C-terminus of the protected variant of Fragment 4 is amidated, and the N-terminus of the protected variant of Fragment 3 is protected (preferably Fmoc- or Trt-protected, most preferably Trt-protected).

In preferred embodiments, the protected variant of Fragment 4 has a free amino group at the N-terminus and does not have a free carboxyl group at the C-terminus, and the protected variant of Fragment 3 has a free carboxyl group at the C-terminus and does not have a free amino group at the N-terminus. In particularly preferred embodiments, the protected variant of Fragment 4 has a free amino group at the N-terminus and an amidated C-terminus, and the protected variant of Fragment 3 has a free carboxyl group at the C-terminus and a protected (preferably Fmoc- or Trt-protected, most preferably Trt-protected) N-terminus.

In some embodiments, the protected variant of Fragment 4 has a free amino group at the N-terminus and an amidated C-terminus, and the protected variant of Fragment 3 has a free carboxyl group at the C-terminus and an Fmoc-protected N-terminus. In some embodiments, the protected variant of Fragment 4 has a free amino group at the N-terminus and an amidated C-terminus, and the protected variant of Fragment 3 has a free carboxyl group at the C-terminus and a Trt-protected N-terminus.

In some embodiments, the protected variant of Fragment 3 is Fmoc-Trp(Boc)-Leu-Val-Lys(Boc)-Gly7-OH (SEQ ID NO: 56).

In some embodiments, the protected variant of Fragment 4 is Ser(tBu)-Gly4-Ser(tBu)-Gly4-Ser(tBu)-Lys(ivDde)-NH₂ (SEQ ID NO: 109).

In some embodiments, the protected variant of Fragment 3 is Fmoc-Trp(Boc)-Leu-Val-Lys(Boc)-Gly7-OH (SEQ ID NO: 56), and the protected variant of Fragment 4 is Ser(tBu)-Gly4-Ser(tBu)-Gly4-Ser(tBu)-Lys(ivDde)-NH₂ (SEQ ID NO: 109).

In some embodiments, the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the coupling composition comprises DIC/Oxyma, HCTU/DIEA, or PyOxim/DIEA. In some embodiments, the coupling composition comprises DIC/Oxyma, HCTU/DIEA, or PyOxim/DIEA in DSMO.

In some embodiments, the coupling composition comprises DIC and Oxyma. In some embodiments, the coupling composition comprises DIC and Oxyma in DMSO.

In some embodiments, the protected variant of Fragment 3 is Fmoc-Trp(Boc)-Leu-Val-Lys(Boc)-Gly7-OH (SEQ ID NO: 56), the protected variant of Fragment 4 is Ser(tBu)-Gly4-Ser(tBu)-Gly4-Ser(tBu)-Lys(ivDde)-NH₂ (SEQ ID NO: 109), and the coupling composition comprises TBTU/collidine, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the protected variant of Fragment 3 is Fmoc-Trp(Boc)-Leu-Val-Lys(Boc)-Gly7-OH (SEQ ID NO: 56), the protected variant of Fragment 4 is Ser(tBu)-Gly4-Ser(tBu)-Gly4-Ser(tBu)-Lys(ivDde)-NH₂ (SEQ ID NO: 109), and the coupling composition comprises DIC/Oxyma, HCTU/DIEA, or PyOxim/DIEA (preferably DIC/Oxyma). In some embodiments, the coupling composition comprises DIC/Oxyma, HCTU/DIEA, or PyOxim/DIEA (preferably DIC/Oxyma) in DMSO.

In some embodiments, the protected variant of Fragment 3 is Fmoc-Trp(Boc)-Leu-Val-Lys(Boc)-Gly7-OH (SEQ ID NO: 56), the protected variant of Fragment 4 is Ser(tBu)-Gly4-Ser(tBu)-Gly4-Ser(tBu)-Lys(ivDde)-NH₂ (SEQ ID NO: 109), and the coupling composition comprises DIC and Oxyma. In some embodiments, the coupling composition comprises DIC and Oxyma in DMSO.

In some embodiments, the coupling occurs at a temperature in the range of 0° C. to 60° C. In some embodiments, the coupling occurs at a temperature in the range of 20° C. to 50° C. In some embodiments, the coupling occurs at a temperature in the range of 20° C. to 30° C. In some embodiments, the coupling occurs at a temperature in the range of 30° C. to 50° C. In some embodiments, the coupling occurs at a temperature in the range of 35° C. to 45° C.

In some embodiments, the method comprises coupling the protected variant of Fragment 3 and the protected variant of Fragment 4 for a time in the range of 1 hour to 72 hours. In some embodiments, the time is in the range of 6 hours to 24 hours.

In some embodiments, the coupling occurs at a temperature in the range of 0° C. to 60° C. for a time in the range of 1 hour to 72 hours. In some embodiments, the coupling occurs at a temperature in the range of 0° C. to 60° C. for a time in the range of 6 hours to 24 hours. In some embodiments, the coupling occurs at a temperature in the range of 20° C. to 50° C. for a time in the range of 1 hour to 72 hours. In some embodiments, the coupling occurs at a temperature in the range of 20° C. to 50° C. for a time in the range of 6 hours to 24 hours.

In some embodiments, the protected variant of Fragment 3 is Fmoc-Trp(Boc)-Leu-Val-Lys(Boc)-Gly7-OH (SEQ ID NO: 56), the protected variant of Fragment 4 is Ser(tBu)-Gly4-Ser(tBu)-Gly4-Ser(tBu)-Lys(ivDde)-NH₂ (SEQ ID NO: 109), the coupling composition comprises TBTU/collidine, PyBOP/TEA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine (preferably DIC/Oxyma), and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours).

In some embodiments, the protected variant of Fragment 3 is Fmoc-Trp(Boc)-Leu-Val-Lys(Boc)-Gly7-OH (SEQ ID NO: 56), the protected variant of Fragment 4 is Ser(tBu)-Gly4-Ser(tBu)-Gly4-Ser(tBu)-Lys(ivDde)-NH₂ (SEQ ID NO: 109), the coupling composition comprises DIC/Oxyma, PyBOP/collidine, PyOxim/collidine, or TBTU/collidine (preferably DIC/Oxyma), and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours).

In some embodiments, the method further comprises selectively deprotecting the N-terminus of the protected variant of Peptide E (e.g., using an Fmoc deprotection method described herein).

Also provided herein is a method of preparing Peptide B or a protected variant thereof, comprising coupling a protected variant of Peptide E with an N-protected alanine to form a protected variant of Peptide B, wherein Peptide E comprises the amino acid sequence of SEQ ID NO: 8 and Peptide B comprises the amino acid sequence of SEQ ID NO: 9.

In some embodiments, the N-protected alanine is coupled with the protected variant of Peptide E in solution. In some embodiments, the N-protected alanine is coupled with the protected variant of Peptide E under liquid phase peptide coupling conditions. In some embodiments, the N-protected alanine is coupled with the protected variant of Peptide E under classical solution phase peptide coupling conditions. In some embodiments, the N-protected alanine is coupled with the protected variant of Peptide E under tag-assisted liquid phase peptide coupling conditions.

In other embodiments, the N-protected alanine is coupled with the protected variant of Peptide E on solid support. In some embodiments, the N-protected alanine is coupled with the protected variant of Peptide E under solid phase peptide coupling conditions.

In some embodiments, a C-terminal carboxyl group of the N-protected alanine is pre-activated. In other embodiments, a C-terminal carboxyl group of the N-protected alanine is activated in situ.

In some embodiments, the method comprises:
contacting the N-protected alanine with a coupling composition to form an N-protected alanine with an activated C-terminal carboxyl group (“activated alanine”); and
contacting the protected variant of Peptide E with the activated alanine to form the protected variant of Peptide B.

In some embodiments, the method comprises:
contacting the N-protected alanine with a coupling composition on solid support to form an N-protected alanine with an activated C-terminal carboxyl group (“activated alanine”); and
contacting the protected variant of Peptide E with the activated alanine on solid support to form the protected variant of Peptide B.

In some embodiments, the method comprises:
contacting the N-protected alanine with a coupling composition in solution to form an N-protected alanine with an activated C-terminal carboxyl group (“activated alanine”); and
contacting the protected variant of Peptide E with the activated alanine in solution to form the protected variant of Peptide B.

In some embodiments (e.g., certain embodiments in which a C-terminal carboxyl group of the N-protected alanine is pre-activated), the method comprises:
contacting the N-protected alanine with a coupling composition to form an N-protected alanine with an activated C-terminal carboxyl group (“activated alanine”); and
contacting the protected variant of Peptide E with the activated alanine to form the protected variant of Peptide B, wherein the contacting does not occur in the presence of a coupling reagent.

In some embodiments, the N-protected alanine is present in molar excess relative to the coupling reagent in the coupling composition during the contacting of the N-protected alanine and the coupling composition.

In some embodiments (e.g., certain embodiments in which a C-terminal carboxyl group of the N-protected alanine is pre-activated), the method comprises:
contacting the N-protected alanine with a coupling composition on solid support to form an N-protected alanine with an activated C-terminal carboxyl group (“activated alanine”); and
contacting the protected variant of Peptide E with the activated alanine on solid support to form the protected variant of Peptide B, wherein the contacting does not occur in the presence of a coupling reagent.

In some embodiments, the N-protected alanine is present in molar excess relative to the coupling reagent in the coupling composition during the contacting of the N-protected alanine and the coupling composition.

In some embodiments (e.g., certain embodiments in which a C-terminal carboxyl group of the N-protected alanine is pre-activated), the method comprises:
contacting the N-protected alanine with a coupling composition in solution to form an N-protected alanine with an activated C-terminal carboxyl group (“activated alanine”); and
contacting the protected variant of Peptide E with the activated alanine in solution to form the protected variant of Peptide B, wherein the contacting does not occur in the presence of a coupling reagent.

In some embodiments, the N-protected alanine is present in molar excess relative to the coupling reagent in the coupling composition during the contacting of the N-protected alanine and the coupling composition.

In some embodiments, the contacting of the N-protected alanine with the coupling composition and the contacting of the activated alanine with the protected variant of Peptide E occur in the same reaction vessel. In other embodiments, the contacting of the N-protected alanine with the coupling composition and the contacting of the activated alanine with the protected variant of Peptide E occur in different reaction vessels.

In some embodiments, the activated alanine is contacted with the protected variant of Peptide E in the presence of a base and/or a coupling additive but not a coupling reagent (such as, e.g., in certain embodiments in which a C-terminal carboxyl group of the N-protected alanine is pre-activated and the N-protected alanine is present in molar excess relative to the coupling reagent in the coupling composition during the contacting to form activated alanine).

In some embodiments in which a C-terminal carboxyl group of the N-protected alanine is pre-activated, the protected variant of Peptide E has a free N-terminal amino group and a free C-terminal carboxyl group.

In some embodiments, the coupling composition comprises a coupling reagent and a base. In some embodiments, the coupling composition comprises a coupling reagent (e.g., a carbodiimide, e.g., DIC, DCC, EDC) and a coupling additive (e.g., HOBt, 5-Cl—HOBt, Oxyma).

In some embodiments, the coupling composition comprises a coupling reagent, a coupling additive, and a base.

In some embodiments, the coupling reagent is a carbodiimide, such as, e.g., DIC, DCC, or EDC. In other embodiments, the coupling reagent is BOP—Cl.

In some embodiments (e.g., certain embodiments in which a C-terminal carboxyl group of the N-protected alanine is activated in situ), the method comprises coupling the N-protected alanine with the protected variant of Peptide E in the presence of a coupling composition. In some embodiments, the method comprises coupling the N-protected alanine with the protected variant of Peptide E in solution in the presence of a coupling composition.

In some embodiments, the protected variant of Peptide E does not have a free carboxyl group at the C-terminus. In some embodiments, the C-terminus of the protected variant of Peptide E is protected. In some embodiments, the C-terminus of the protected variant of Peptide E is amidated. In some embodiments, the protected variant of Peptide E has a free amino group at the N-terminus. In preferred embodiments, the protected variant of Peptide E has a free amino group at the N-terminus and does not have a free carboxyl group at the C-terminus. In particularly preferred embodiments, the protected variant of Peptide E has a free amino group at the N-terminus and an amidated C-terminus.

In some embodiments, the N-protected alanine is Fmoc-protected. In some embodiments, the N-protected alanine has a free carboxyl group at the C-terminus. In preferred embodiments, the N-protected alanine has a free carboxyl group at the C-terminus and an Fmoc-protected N-terminus.

In some embodiments, the protected variant of Peptide E has a free amino group at the N-terminus and does not have a free carboxyl group at the C-terminus, and the N-protected alanine has a free carboxyl group at the C-terminus and an Fmoc-protected N-terminus.

In some embodiments, the C-terminus of the protected variant of Peptide E is amidated, and the N-terminus of the N-protected alanine is Fmoc-protected.

In preferred embodiments, the protected variant of Peptide E has a free amino group at the N-terminus and does not have a free carboxyl group at the C-terminus, and the N-protected alanine has a free carboxyl group at the C-terminus and an Fmoc- or Trt-protected N-terminus. In particularly preferred embodiments, the protected variant of Peptide E has a free amino group at the N-terminus and an amidated C-terminus, and the N-protected alanine has a free carboxyl group at the C-terminus and an Fmoc-protected N-terminus.

In some embodiments, the protected variant of Peptide E is Trp(Boc)-Leu-Val-Lys(Boc)-Gly7-Ser(tBu)-Gly4-Ser(tBu)-Gly4-Ser(tBu)-Lys(ivDde)-NH₂ (SEQ ID NO: 160).

In some embodiments, the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the N-protected alanine is Fmoc- or Trt-protected, the protected variant of Peptide E is Trp(Boc)-Leu-Val-Lys(Boc)-Gly7-Ser(tBu)-Gly4-Ser(tBu)-Gly4-Ser(tBu)-Lys(ivDde)-NH₂ (SEQ ID NO: 160), and the coupling composition comprises TBTU/collidine, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the N-protected alanine is Fmoc-protected, the protected variant of Peptide E is Trp(Boc)-Leu-Val-Lys(Boc)-Gly7-Ser(tBu)-Gly4-Ser(tBu)-Gly4-Ser(tBu)-Lys(ivDde)-NH₂ (SEQ ID NO: 160), and the coupling composition comprises TBTU/collidine, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the N-protected alanine is Trt-protected, the protected variant of Peptide E is Trp(Boc)-Leu-Val-Lys(Boc)-Gly7-Ser(tBu)-Gly4-Ser(tBu)-Gly4-Ser(tBu)-Lys(ivDde)-NH₂ (SEQ ID NO: 160), and the coupling composition comprises TBTU/collidine, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the coupling occurs at a temperature in the range of 0° C. to 60° C. In some embodiments, the coupling occurs at a temperature in the range of 20° C. to 50° C. In some embodiments, the coupling occurs at a temperature in the range of 20° C. to 30° C. In some embodiments, the coupling occurs at a temperature in the range of 30° C. to 50° C. In some embodiments, the coupling occurs at a temperature in the range of 35° C. to 45° C.

In some embodiments, the method comprises coupling the N-protected alanine and the protected variant of Peptide E for a time in the range of 1 hour to 72 hours. In some embodiments, the time is in the range of 6 hours to 24 hours.

In some embodiments, the coupling occurs at a temperature in the range of 0° C. to 60° C. for a time in the range of 1 hour to 72 hours. In some embodiments, the coupling occurs at a temperature in the range of 0° C. to 60° C. for a time in the range of 6 hours to 24 hours. In some embodiments, the coupling occurs at a temperature in the range of 20° C. to 50° C. for a time in the range of 1 hour to 72 hours. In some embodiments, the coupling occurs at a temperature in the range of 20° C. to 50° C. for a time in the range of 6 hours to 24 hours.

In some embodiments, the N-protected alanine is Fmoc- or Trt-protected, the protected variant of Peptide E is Trp(Boc)-Leu-Val-Lys(Boc)-Gly7-Ser(tBu)-Gly4-Ser(tBu)-Gly4-Ser(tBu)-Lys(ivDde)-NH₂ (SEQ ID NO: 160), the coupling composition comprises TBTU/collidine, PyBOP/TEA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine, and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours).

In some embodiments, the N-protected alanine is Fmoc-protected, the protected variant of Peptide E is Trp(Boc)-Leu-Val-Lys(Boc)-Gly7-Ser(tBu)-Gly4-Ser(tBu)-Gly4-Ser(tBu)-Lys(ivDde)-NH₂ (SEQ ID NO: 160), the coupling composition comprises TBTU/collidine, PyBOP/TEA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine, and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours).

In some embodiments, the N-protected alanine is Trt-protected, the protected variant of Peptide E is Trp(Boc)-Leu-Val-Lys(Boc)-Gly7-Ser(tBu)-Gly4-Ser(tBu)-Gly4-Ser(tBu)-Lys(ivDde)-NH₂ (SEQ ID NO: 160), the coupling composition comprises TBTU/collidine, PyBOP/TEA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or

Oxyma/PyOxim/collidine, and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours).

In some embodiments, the method further comprises selectively deprotecting the N-terminus of the protected variant of Peptide B (e.g., using an Fmoc or Trt deprotection method described herein).

Also provided herein is a method of preparing Peptide B or a protected variant thereof, comprising:
coupling or having coupled a protected variant of Fragment 3 with a protected variant of Fragment 4 to form a first protected variant of Peptide E, wherein the protected variant of Fragment 3 has a free carboxyl group at the C-terminus and a protected (preferably Fmoc-protected) N-terminus, the protected variant of Fragment 4 has a free amino group at the N-terminus, Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8;
selectively deprotecting or having selectively deprotected the N-terminus of the first protected variant of Peptide E to form a second protected variant of Peptide E with a free amino group at the N-terminus; and
coupling or having coupled the second protected variant of Peptide E with an N-protected alanine to form a protected variant of Peptide B, wherein Peptide B comprises the amino acid sequence of SEQ ID NO: 9.

In some embodiments, the protected variant of Fragment 3 and the protected variant of Fragment 4 are coupled under conditions described herein.

In some embodiments, the second protected variant of Peptide E and the N-protected alanine are coupled under conditions described herein.

In some embodiments, the protected variant of Fragment 3 and the protected variant of Fragment 4 are coupled under conditions described above, and the second protected variant of Peptide E and the N-protected alanine are coupled under conditions described herein.

In some embodiments, the N-terminus of the first protected variant of Peptide E is Fmoc-protected, and the selective deprotection comprises contacting the first protected variant of Peptide E with an amine base to selectively remove the Fmoc protecting group at the N-terminus. In some embodiments, the amine base is selected from piperidine, piperazine, and morpholine (e.g., 5-30% (v/v) piperidine, piperazine, or morpholine). In some embodiments, the amine base is piperidine. In some embodiments, the N-terminus of the first protected variant of Peptide E is Fmoc-protected, and the selective deprotection comprises contacting the first protected variant of Peptide E with piperidine in DMF, optionally in the presence of DBU.

In some embodiments, the N-terminus of the first protected variant of Peptide E is Boc-protected, and the selective deprotection comprises contacting the first protected variant of Peptide E with an acid (preferably an acid selected from TFA, HCl, and HBr) to selectively remove the Boc protecting group at the N-terminus. In some embodiments, the first protected variant of Peptide E is contacted with TFA (e.g., neat TFA or TFA in DCM, e.g., 20-50% (v/v) TFA in DCM). In some embodiments, the first protected variant of Peptide E is contacted with HCl (e.g., HCl in DCM, DCE, toluene, or dioxane, e.g., 3-4 M HCl in DCM or dioxane).

In some embodiments, the N-terminus of the first protected variant of Peptide E is Boc-protected, and the selective deprotection comprises contacting the first protected variant of Peptide E with an acid (preferably an acid selected from TFA, HCl, and HBr) in the presence of a scavenger. In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some preferred embodiments, the scavenger is a hydrosilane. In some particularly preferred embodiments, the scavenger is TIS. In some embodiments, the first protected variant of Peptide E is contacted with TFA (e.g., neat TFA or TFA in DCM, e.g., 20-50% (v/v) TFA in DCM) in the presence of a scavenger (preferably TIS). In some embodiments, the first protected variant of Peptide E is contacted with HCl (e.g., HCl in DCM, DCE, toluene, or dioxane, e.g., 3-4 M HCl in DCM or dioxane) in the presence of a scavenger.

In some embodiments, the N-terminus of the first protected variant of Peptide E is Trt-protected, and the selective deprotection comprises contacting the first protected variant of Peptide E with a Brønsted acid or a Lewis acid (preferably TFA, HCl, or dichloroacetic acid, most preferably TFA), optionally in the presence of a scavenger (preferably TIS), to selectively remove the trityl protecting group at the N-terminus. In some embodiments, the first protected variant of Peptide E is contacted with a Brønsted acid (preferably a dilute Brønsted acid). In some embodiments, the Brønsted acid is selected from TFA, HCl, and dichloroacetic acid (preferably dilute TFA, HCl, or dichloroacetic acid). In other embodiments, the first protected variant of Peptide E is contacted with a Lewis acid (preferably a dilute Lewis acid).

In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some embodiments, the scavenger is a hydrosilane. In some embodiments, the scavenger is TIS.

In some embodiments, the N-terminus of the first protected variant of Peptide E is Trt-protected, and the selective deprotection comprises contacting the first protected variant of Peptide E with HCl. In some embodiments, the selective deprotection comprises contacting the first protected variant of Peptide E with dilute HCl.

In some embodiments, the N-terminus of the first protected variant of Peptide E is Trt-protected, and the selective deprotection comprises contacting the first protected variant of Peptide E with TFA in the presence of TIS. In some embodiments, the selective deprotection comprises contacting the first protected variant of Peptide E with dilute TFA in the presence of TIS.

Also provided herein is a method of preparing Peptide D or a protected variant thereof, comprising coupling a protected variant of Fragment 1 with a protected variant of Fragment 2B in to form a protected variant of Peptide D, wherein Peptide D comprises the amino acid sequence of SEQ ID NO: 15, Fragment 1 comprises the amino acid sequence of SEQ ID NO: 1, and Fragment 2B comprises the amino acid sequence of SEQ ID NO: 16.

In some embodiments, the protected variant of Fragment 1 is coupled with the protected variant of Fragment 2B in solution. In some embodiments, the protected variant of Fragment 1 is coupled with the protected variant of Fragment 2B under liquid phase peptide coupling conditions. In some embodiments, the protected variant of Fragment 1 is coupled with the protected variant of Fragment 2B under classical solution phase peptide coupling conditions. In some embodiments, the protected variant of Fragment 1 is coupled with the protected variant of Fragment 2B under tag-assisted liquid phase peptide coupling conditions.

In other embodiments, the protected variant of Fragment 1 is coupled with the protected variant of Fragment 2B on solid support. In some embodiments, the protected variant of Fragment 1 is coupled with the protected variant of Fragment 2B under solid phase peptide coupling conditions.

In some embodiments, a C-terminal carboxyl group of the protected variant of Fragment 1 is pre-activated. In other embodiments, a C-terminal carboxyl group of the protected variant of Fragment 1 is activated in situ.

In some embodiments, the method comprises:
contacting the protected variant of Fragment 1 with a coupling composition to form a protected variant of Fragment 1 with an activated C-terminal carboxyl group (“activated Fragment 1”); and
contacting the protected variant of Fragment 2B with the activated Fragment 1 to form the protected variant of Peptide D.

In some embodiments, the method comprises:
contacting the protected variant of Fragment 1 with a coupling composition on solid support to form a protected variant of Fragment 1 with an activated C-terminal carboxyl group (“activated Fragment 1”); and
contacting the protected variant of Fragment 2B with the activated Fragment 1 on solid support to form the protected variant of Peptide D.

In some embodiments, the method comprises:
contacting the protected variant of Fragment 1 with a coupling composition in solution to form a protected variant of Fragment 1 with an activated C-terminal carboxyl group (“activated Fragment 1”); and
contacting the protected variant of Fragment 2B with the activated Fragment 1 in solution to form the protected variant of Peptide D.

In some embodiments (e.g., certain embodiments in which a C-terminal carboxyl group of the protected variant of Fragment 1 is pre-activated), the method comprises:
contacting the protected variant of Fragment 1 with a coupling composition to form a protected variant of Fragment 1 with an activated C-terminal carboxyl group (“activated Fragment 1”); and
contacting the protected variant of Fragment 2B with the activated Fragment 1 to form the protected variant of Peptide D, wherein the contacting does not occur in the presence of a coupling reagent.

In some embodiments, the protected variant of Fragment 1 is present in molar excess relative to the coupling reagent in the coupling composition during the contacting of the protected variant of Fragment 1 and the coupling composition.

In some embodiments (e.g., certain embodiments in which a C-terminal carboxyl group of the protected variant of Fragment 1 is pre-activated), the method comprises:
contacting the protected variant of Fragment 1 with a coupling composition on solid support to form a protected variant of Fragment 1 with an activated C-terminal carboxyl group (“activated Fragment 1”); and
contacting the protected variant of Fragment 2B with the activated Fragment 1 on solid support to form the protected variant of Peptide D, wherein the contacting does not occur in the presence of a coupling reagent.

In some embodiments, the protected variant of Fragment 1 is present in molar excess relative to the coupling reagent in the coupling composition during the contacting of the protected variant of Fragment 1 and the coupling composition.

In some embodiments (e.g., certain embodiments in which a C-terminal carboxyl group of the protected variant of Fragment 1 is pre-activated), the method comprises:
contacting the protected variant of Fragment 1 with a coupling composition in solution to form a protected variant of Fragment 1 with an activated C-terminal carboxyl group (“activated Fragment 1”); and
contacting the protected variant of Fragment 2B with the activated Fragment 1 in solution to form the protected variant of Peptide D, wherein the contacting does not occur in the presence of a coupling reagent.

In some embodiments, the protected variant of Fragment 1 is present in molar excess relative to the coupling reagent in the coupling composition during the contacting of the protected variant of Fragment 1 and the coupling composition.

In some embodiments, the contacting of the protected variant of Fragment 1 with the coupling composition and the contacting of the activated Fragment 1 with the protected variant of Fragment 2B occur in the same reaction vessel. In other embodiments, the contacting of the protected variant of Fragment 1 with the coupling composition and the contacting of the activated Fragment 1 with the protected variant of Fragment 2B occur in different reaction vessels.

In some embodiments, the activated Fragment 1 is contacted with the protected variant of Fragment 2B in the presence of a base and/or a coupling additive but not a coupling reagent (such as, e.g., in certain embodiments in which a C-terminal carboxyl group of the protected variant of Fragment 1 is pre-activated and the protected variant of Fragment 1 is present in molar excess relative to the coupling reagent in the coupling composition during the contacting to form activated Fragment 1).

In some embodiments in which a C-terminal carboxyl group of the protected variant of Fragment 1 is pre-activated, the protected variant of Fragment 2B has a free N-terminal amino group and a free C-terminal carboxyl group.

In some embodiments, the coupling composition comprises a coupling reagent and a base. In some embodiments, the coupling composition comprises a coupling reagent (e.g., a carbodiimide, e.g., DIC, DCC, EDC) and a coupling additive (e.g., HOBt, 5-Cl—HOBt, Oxyma).

In some embodiments, the coupling composition comprises a coupling reagent, a coupling additive, and a base.

In some embodiments, the coupling reagent is a carbodiimide, such as, e.g., DIC, DCC, or EDC. In other embodiments, the coupling reagent is BOP—Cl.

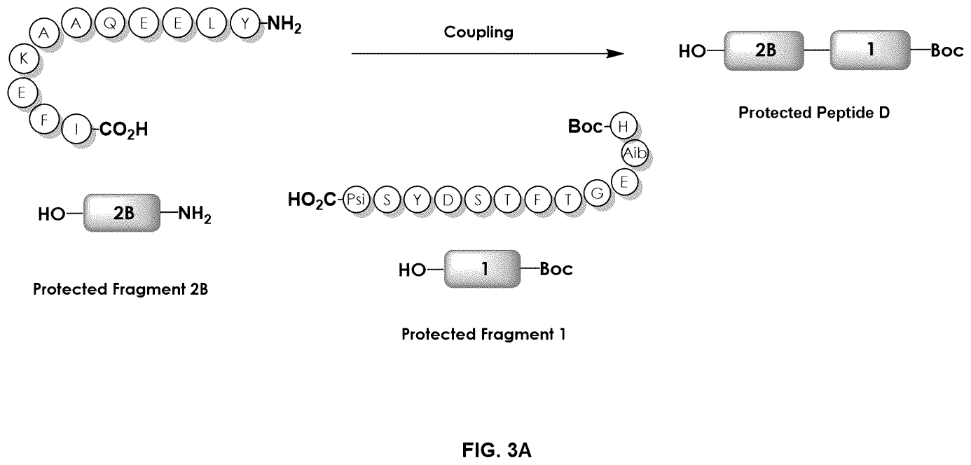

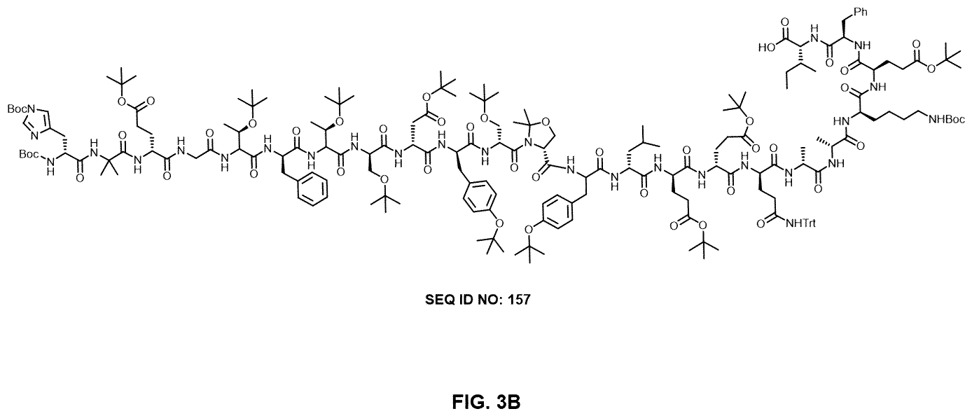

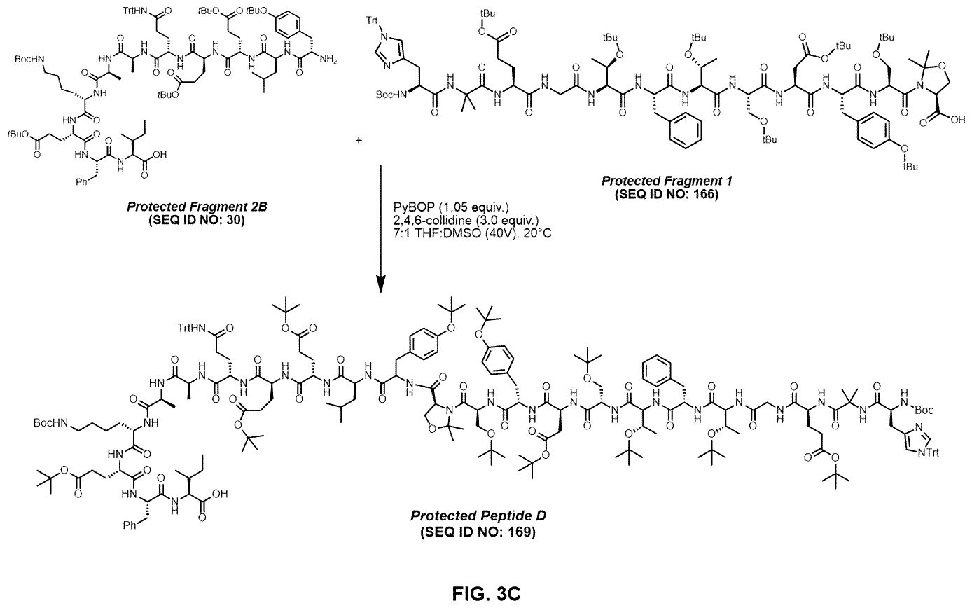

In some embodiments, a protected variant of Peptide D can be prepared in solution by coupling a protected variant of Fragment 2B and a protected variant of Fragment 1 as shown in FIG. 3A. FIG. 3B depicts another protected variant of Peptide D which can be prepared by an analogous process to that shown in FIG. 3A. Alternatively, a protected variant of Peptide D can be efficiently prepared in solution by coupling a protected variant of Fragment 2B and a protected variant of Fragment 1 as shown in FIG. 3C.

In some embodiments, the protected variant of Fragment 1 is Boc-His(Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 166) or Fmoc-His(Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 167), the protected variant of Fragment 2B is Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 30), the coupling composition comprises PyBOP/collidine, DIC/Oxyma, DIC/Oxyma/collidine, T3P/Oxyma, T3P/Oxyma/collidine, HCTU/collidine, HCTU/6-Cl—HOBt/collidine, EDC/HOBt, EDC/HOBt/collidine, HATU/collidine, HBTU/collidine, TBTU/collidine, DMAP/EDC, Oxyma/PyOxim, or Oxyma/PyOxim/collidine (preferably PyBOP/collidine), and the coupling occurs at a temperature in the range of 0° C. to 60° C. (preferably a temperature in the range of 20° C. to 50° C., more preferably a temperature in the range of 20° C. to 30° C.) for a time in the range of 30 minutes to 72 hours (preferably a time in the range of 30 minutes to 24 hours).

In some embodiments (e.g., certain embodiments in which a C-terminal carboxyl group of the protected variant of Fragment 1 is activated in situ), the method comprises coupling the protected variant of Fragment 1 with the protected variant of Fragment 2B in the presence of a coupling composition. In some embodiments, the method comprises coupling the protected variant of Fragment 1 with the protected variant of Fragment 2B in solution in the presence of a coupling composition.

In some embodiments, the protected variant of Fragment 2B does not have a free carboxyl group at the C-terminus. In some embodiments, the C-terminus of the protected variant of Fragment 2B is protected. In some embodiments, the protected variant of Fragment 2B has a free amino group at the N-terminus. In preferred embodiments, the protected variant of Fragment 2B has a free amino group at the N-terminus and does not have a free carboxyl group at the C-terminus. In particularly preferred embodiments, the protected variant of Fragment 2B has a free amino group at the N-terminus and a protected C-terminus.

In some embodiments, the protected variant of Fragment 1 does not have a free amino group at the N-terminus. In some embodiments, the N-terminus of the protected variant of Fragment 1 is protected (preferably Boc-protected). In some embodiments, the protected variant of Fragment 1 has a free carboxyl group at the C-terminus. In preferred embodiments, the protected variant of Fragment 1 has a free carboxyl group at the C-terminus and does not have a free amino group at the N-terminus. In particularly preferred embodiments, the protected variant of Fragment 1 has a free carboxyl group at the C-terminus and a protected (preferably Boc-protected) N-terminus.

In some embodiments, the protected variant of Fragment 2B has a free amino group at the N-terminus and does not have a free carboxyl group at the C-terminus, and the protected variant of Fragment 1 has a free carboxyl group at the C-terminus and a protected (preferably Boc-protected) N-terminus.

In some embodiments, the C-terminus of the protected variant of Fragment 2B is protected, and the N-terminus of the protected variant of Fragment 1 is protected (preferably Boc-protected).

In preferred embodiments, the protected variant of Fragment 2B has a free amino group at the N-terminus and does not have a free carboxyl group at the C-terminus, and the protected variant of Fragment 1 has a free carboxyl group at the C-terminus and does not have a free amino group at the N-terminus. In particularly preferred embodiments, the protected variant of Fragment 2B has a free amino group at the N-terminus and a protected C-terminus, and the protected variant of Fragment 1 has a free carboxyl group at the C-terminus and a protected (preferably Boc-protected) N-terminus.

In some embodiments, the N-terminus, the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, and the side chain of the Ser residue at position 12 of the protected variant of Fragment 1 are protected and the protected variant of Fragment 1 has a free carboxyl group at the C-terminus.

In some embodiments, the side chain of the Tyr residue at position 1, the side chain of the Glu residue at position 3, the side chain of the Glu residue at position 4, the side chain of the Gln residue at position 5, the side chain of the Lys residue at position 8, and the side chain of the Glu residue at position 9 of the protected variant of Fragment 2B are protected and the protected variant of Fragment 2B has a free amino group at the N-terminus.

In some embodiments, the N-terminus, the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, and the side chain of the Ser residue at position 12 of the protected variant of Fragment 1 are protected, the protected variant of Fragment 1 has a free carboxyl group at the C-terminus, the side chain of the Tyr residue at position 1, the side chain of the Glu residue at position 3, the side chain of the Glu residue at position 4, the side chain of the Gln residue at position 5, the side chain of the Lys residue at position 8, and the side chain of the Glu residue at position 9 of the protected variant of Fragment 2B are protected, and the protected variant of Fragment 2B has a free amino group at the N-terminus.

In some embodiments, the side chain of the His residue at position 1 of the protected variant of Fragment 1 is protected with a trityl (Trt) protecting group; the side chain of the Glu residue at position 3 of the protected variant of Fragment 1 is protected with a tert-butyl ester (OtBu) protecting group; the side chains of the Thr residues at position 5 and position 7 of the protected variant of Fragment 1 are each protected with a tert-butyl (tBu) protecting group; the side chains of the Ser residues at position 8 and position 11 of the protected variant of Fragment 1 are each protected with a tBu protecting group; the side chain of the Asp residue at position 9 of the protected variant of Fragment 1 is protected with an OtBu protecting group; the side chain of the Tyr residue at position 10 of the protected variant of Fragment 1 is protected with a tBu protecting group; and the Ser residue at position 12 of the protected variant of Fragment 1 is protected as a dimethylated pseudoproline (Psi(Me,Me) pro) moiety in which the oxazolidine is derived from Ser.

In some embodiments, the side chain of the Tyr residue at position 1 of the protected variant of Fragment 2B is protected with a tBu protecting group; the side chains of the Glu residues at position 3, position 4, and position 9 of the protected variant of Fragment 2B are each protected with a OtBu protecting group; the side chain of the Gln residue at position 5 of the protected variant of Fragment 2B is protected with a Trt protecting group; and the side chain of the Lys residue at position 8 of the protected variant of Fragment 2B is protected with a tert-butyloxycarbonyl (Boc) protecting group.

In some embodiments, the side chain of the His residue at position 1 of the protected variant of Fragment 1 is protected with a trityl (Trt) protecting group; the side chain of the Glu residue at position 3 of the protected variant of Fragment 1 is protected with a tert-butyl ester (OtBu) protecting group; the side chains of the Thr residues at position 5 and position 7 of the protected variant of Fragment 1 are each protected with a tert-butyl (tBu) protecting group; the side chains of the Ser residues at position 8 and position 11 of the protected variant of Fragment 1 are each protected with a tBu protecting group; the side chain of the Asp residue at position 9 of the protected variant of Fragment 1 is protected with an OtBu protecting group; the side chain of the Tyr residue at position 10 of the protected variant of Fragment 1 is protected with a tBu protecting group; the Ser residue at position 12 of the protected variant of Fragment 1 is protected as a dimethylated pseudoproline (Psi(Me, Me) pro) moiety in which the oxazolidine is derived from Ser; the side chain of the Tyr residue at position 1 of the protected variant of Fragment 2B is protected with a tBu protecting group; the side chains of the Glu residues at position 3, position 4, and position 9 of the protected variant of Fragment 2B are each protected with a OtBu protecting group; the side chain of the Gln residue at position 5 of the protected variant of Fragment 2B is protected with a Trt protecting group; and the side chain of the Lys residue at position 8 of the protected variant of Fragment 2B is protected with a tert-butyloxycarbonyl (Boc) protecting group.

In some embodiments, the protected variant of Fragment 1 is Boc-His(Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 166) or Fmoc-His(Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 167). In some embodiments, the protected variant of Fragment 1 is Boc-His(Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 166). In some embodiments, the protected variant of Fragment 1 is Fmoc-His(Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 167).

In some embodiments, the protected variant of Fragment 2B is Tyr(tBu)-Leu-Glu(OtBu)-Glu(OtBu)-Gln(Trt)-Ala-Ala-Lys(Boc)-Glu(OtBu)-Phe-Ile-X, wherein X is a C-terminal protecting group (SEQ ID NO: 46).

In some embodiments, the protected variant of Fragment 1 is Boc-His(Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 166) or Fmoc-His(Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 167), and the protected variant of Fragment 2B is Tyr(tBu)-Leu-Glu(OtBu)-Glu(OtBu)-Gln(Trt)-Ala-Ala-Lys(Boc)-Glu(OtBu)-Phe-Ile-X, wherein X is a C-terminal protecting group (SEQ ID NO: 46). In some embodiments, the protected variant of Fragment 1 is Boc-His(Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 166), and the protected variant of Fragment 2B is Tyr(tBu)-Leu-Glu(OtBu)-Glu(OtBu)-Gln(Trt)-Ala-Ala-Lys(Boc)-Glu(OtBu)-Phe-Ile-X, wherein X is a C-terminal protecting group (SEQ ID NO: 46). In some embodiments, the protected variant of Fragment 1 is Fmoc-His(Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me, Me)Pro)-OH (SEQ ID NO: 167), and the protected variant of Fragment 2B is Tyr(tBu)-Leu-Glu(OtBu)-Glu(OtBu)-Gln(Trt)-Ala-Ala-Lys(Boc)-Glu(OtBu)-Phe-Ile-X, wherein X is a C-terminal protecting group (SEQ ID NO: 46).

In some embodiments, the coupling composition comprises 1 to 10 equivalents of a coupling reagent, optionally 1 to 10 equivalents of a base, and optionally 1 to 10 equivalents of a coupling additive, wherein equivalents are relative to the protected variant of Fragment 2B. In some embodiments, the coupling composition comprises 1 to 5 equivalents of a coupling reagent, optionally 1 to 5 equivalents of a base, and optionally 1 to 5 equivalents of a coupling additive, wherein equivalents are relative to the protected variant of Fragment 2B. In some embodiments, the coupling composition comprises 2 to 10 equivalents of a coupling reagent, optionally 2 to 10 equivalents of a base, and optionally 2 to 10 equivalents of a coupling additive, wherein equivalents are relative to the protected variant of Fragment 2B.

In some embodiments, the coupling composition comprises PyBOP/collidine, DIC/Oxyma, DIC/Oxyma/collidine, T3P/Oxyma, T3P/Oxyma/collidine, HCTU/collidine, HCTU/6-Cl—HOBt/collidine, EDC/HOBt, EDC/HOBt/collidine, HATU/collidine, HBTU/collidine, TBTU/collidine, DMAP/EDC, Oxyma/PyOxim, or Oxyma/PyOxim/collidine. In some embodiments, the coupling composition comprises PyBOP/collidine, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the coupling composition comprises PyBOP and collidine. In some embodiments, the coupling composition comprises PyBOP and collidine in THF and DMSO.

In some embodiments, the coupling composition comprises EDC and HOBt. In some embodiments, the coupling composition comprises EDC, HOBt, and collidine.

In some embodiments, the coupling occurs at a temperature in the range of 0° C. to 60° C. In some embodiments, the coupling occurs at a temperature in the range of 20° C. to 50° C. In some embodiments, the coupling occurs at a temperature in the range of 20° C. to 30° C. In some embodiments, the coupling occurs at a temperature in the range of 30° C. to 50° C. In some embodiments, the coupling occurs at a temperature in the range of 35° C. to 45° C.

In some embodiments, the method comprises coupling the protected variant of Fragment 1 and the protected variant of Fragment 2B for a time in the range of 30 minutes to 72 hours. In some embodiments, the time is in the range of 30 minutes to 24 hours.

In some embodiments, the coupling occurs at a temperature in the range of 0° C. to 60° C. for a time in the range of 30 minutes to 72 hours. In some embodiments, the coupling occurs at a temperature in the range of 0° C. to 60° C. for a time in the range of 30 minutes to 24 hours. In some embodiments, the coupling occurs at a temperature in the range of 20° C. to 50° C. for a time in the range of 30 minutes to 72 hours. In some embodiments, the coupling occurs at a temperature in the range of 20° C. to 50° C. for a time in the range of 30 minutes to 24 hours.

In some embodiments, the protected variant of Fragment 1 is Boc-His(Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 166) or Fmoc-His(Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 167), the protected variant of Fragment 2B is Tyr(tBu)-Leu-Glu(OtBu)-Glu(OtBu)-Gln(Trt)-Ala-Ala-Lys(Boc)-Glu(OtBu)-Phe-Ile-X, wherein X is a C-terminal protecting group (SEQ ID NO: 46), the coupling composition comprises PyBOP/collidine, DIC/Oxyma, DIC/Oxyma/collidine, T3P/Oxyma, T3P/Oxyma/collidine, HCTU/collidine, HCTU/6-Cl—HOBt/collidine, EDC/HOBt, EDC/HOBt/collidine, HATU/collidine, HBTU/collidine, TBTU/collidine, DMAP/EDC, Oxyma/PyOxim, or Oxyma/PyOxim/collidine (preferably PyBOP/collidine), and the coupling occurs at a temperature in the range of 0° C. to 60° C. (preferably a temperature in the range of 20° C. to 50° C., more preferably a temperature in the range of 20° C. to 30° C.) for a time in the range of 30 minutes to 72 hours (preferably a time in the range of 30 minutes to 24 hours).

In some embodiments, the method further comprises selectively deprotecting the N-terminus of the protected variant of Peptide D (e.g., using a Boc or Fmoc deprotection method described herein).

Also provided herein is a method of preparing Peptide A or a protected variant thereof, comprising coupling a protected variant of Peptide B with a protected variant of Peptide D to form a protected variant of Peptide A, wherein Peptide B comprises the amino acid sequence of SEQ ID NO: 9, Peptide D comprises the amino acid sequence of SEQ ID NO: 15, and Peptide A comprises the amino acid sequence of SEQ ID NO: 7. In some embodiments, the method further comprises deprotecting the protected variant of Peptide A to isolate Peptide A. In some embodiments, the method further comprises adding a bromoacetyl moiety to the Lys at the C-terminus of Peptide A or a protected variant thereof.

In some embodiments, the protected variant of Peptide B is coupled with the protected variant of Peptide D in solution. In some embodiments, the protected variant of Peptide B is coupled with the protected variant of Peptide D under liquid phase peptide coupling conditions. In some embodiments, the protected variant of Peptide B is coupled with the protected variant of Peptide D under classical solution phase peptide coupling conditions. In some embodiments, the protected variant of Peptide B is coupled with the protected variant of Peptide D under tag-assisted liquid phase peptide coupling conditions.

In other embodiments, the protected variant of Peptide B is coupled with the protected variant of Peptide D on solid support. In some embodiments, the protected variant of Peptide B is coupled with the protected variant of Peptide D under solid phase peptide coupling conditions.

In some embodiments, a C-terminal carboxyl group of the protected variant of Peptide D is pre-activated. In other embodiments, a C-terminal carboxyl group of the protected variant of Peptide D is activated in situ.

In some embodiments, the method comprises:
contacting the protected variant of Peptide D with a coupling composition to form a protected variant of Peptide D with an activated C-terminal carboxyl group (“activated Peptide D”); and
contacting the protected variant of Peptide B with the activated Peptide D to form the protected variant of Peptide A.

In some embodiments, the method comprises:
contacting the protected variant of Peptide D with a coupling composition on solid support to form a protected variant of Peptide D with an activated C-terminal carboxyl group (“activated Peptide D”); and
contacting the protected variant of Peptide B with the activated Peptide D on solid support to form the protected variant of Peptide A.

In some embodiments, the method comprises:
contacting the protected variant of Peptide D with a coupling composition in solution to form a protected variant of Peptide D with an activated C-terminal carboxyl group (“activated Peptide D”); and
contacting the protected variant of Peptide B with the activated Peptide D in solution to form the protected variant of Peptide A.

In some embodiments (e.g., certain embodiments in which a C-terminal carboxyl group of the protected variant of Peptide D is pre-activated), the method comprises:
contacting the protected variant of Peptide D with a coupling composition to form a protected variant of Peptide D with an activated C-terminal carboxyl group (“activated Peptide D”); and
contacting the protected variant of Peptide B with the activated Peptide D to form the protected variant of Peptide A, wherein the contacting does not occur in the presence of a coupling reagent.

In some embodiments, the protected variant of Peptide D is present in molar excess relative to the coupling reagent in the coupling composition during the contacting of the protected variant of Peptide D and the coupling composition.

In some embodiments (e.g., certain embodiments in which a C-terminal carboxyl group of the protected variant of Peptide D is pre-activated), the method comprises:
contacting the protected variant of Peptide D with a coupling composition on solid support to form a protected variant of Peptide D with an activated C-terminal carboxyl group (“activated Peptide D”); and
contacting the protected variant of Peptide B with the activated Peptide D on solid support to form the protected variant of Peptide A, wherein the contacting does not occur in the presence of a coupling reagent.

In some embodiments, the protected variant of Peptide D is present in molar excess relative to the coupling reagent in the coupling composition during the contacting of the protected variant of Peptide D and the coupling composition.

In some embodiments (e.g., certain embodiments in which a C-terminal carboxyl group of the protected variant of Peptide D is pre-activated), the method comprises:
contacting the protected variant of Peptide D with a coupling composition in solution to form a protected variant of Peptide D with an activated C-terminal carboxyl group (“activated Peptide D”); and
contacting the protected variant of Peptide B with the activated Peptide D in solution to form the protected variant of Peptide A, wherein the contacting does not occur in the presence of a coupling reagent.

In some embodiments, the protected variant of Peptide D is present in molar excess relative to the coupling reagent in the coupling composition during the contacting of the protected variant of Peptide D and the coupling composition.

In some embodiments, the contacting of the protected variant of Peptide D with the coupling composition and the contacting of the activated Peptide D with the protected variant of Peptide B occur in the same reaction vessel. In other embodiments, the contacting of the protected variant of Peptide D with the coupling composition and the contacting of the activated Peptide D with the protected variant of Peptide B occur in different reaction vessels.

In some embodiments, the activated Peptide D is contacted with the protected variant of Peptide B in the presence of a base and/or a coupling additive but not a coupling reagent (such as, e.g., in certain embodiments in which a C-terminal carboxyl group of the protected variant of Peptide D is pre-activated and the protected variant of Peptide D is present in molar excess relative to the coupling reagent in the coupling composition during the contacting to form activated Peptide D).

In some embodiments in which a C-terminal carboxyl group of the protected variant of Peptide D is pre-activated, the protected variant of Peptide B has a free N-terminal amino group and a free C-terminal carboxyl group.

In some embodiments, the coupling composition comprises a coupling reagent and a base. In some embodiments, the coupling composition comprises a coupling reagent (e.g., a carbodiimide, e.g., DIC, DCC, EDC) and a coupling additive (e.g., HOBt, 5-Cl—HOBt, Oxyma).

In some embodiments, the coupling composition comprises a coupling reagent, a coupling additive, and a base.

In some embodiments, the coupling reagent is a carbodiimide, such as, e.g., DIC, DCC, or EDC. In other embodiments, the coupling reagent is BOP—Cl.

In some embodiments (e.g., certain embodiments in which a C-terminal carboxyl group of the protected variant of Peptide D is activated in situ), the method comprises coupling the protected variant of Peptide D with the protected variant of Peptide B in the presence of a coupling composition. In some embodiments, the method comprises coupling the protected variant of Peptide D with the protected variant of Peptide B in solution in the presence of a coupling composition.

In some embodiments, the protected variant of Peptide B does not have a free carboxyl group at the C-terminus. In some embodiments, the C-terminus of the protected variant of Peptide B is protected. In some embodiments, the C-terminus of the protected variant of Peptide B is amidated. In some embodiments, the protected variant of Peptide B has a free amino group at the N-terminus. In preferred embodiments, the protected variant of Peptide B has a free amino group at the N-terminus and does not have a free carboxyl group at the C-terminus. In particularly preferred embodiments, the protected variant of Peptide B has a free amino group at the N-terminus and an amidated C-terminus.

In some embodiments, the protected variant of Peptide D does not have a free amino group at the N-terminus. In some embodiments, the N-terminus of the protected variant of Peptide D is protected (preferably Boc-protected). In some embodiments, the protected variant of Peptide D has a free carboxyl group at the C-terminus. In preferred embodiments, the protected variant of Peptide D has a free carboxyl group at the C-terminus and does not have a free amino group at the N-terminus. In particularly preferred embodiments, the protected variant of Peptide D has a free carboxyl group at the C-terminus and a protected (preferably Boc-protected) N-terminus.

In some embodiments, the protected variant of Peptide B has a free amino group at the N-terminus and does not have a free carboxyl group at the C-terminus, and the protected variant of Peptide D has a free carboxyl group at the C-terminus and a protected (preferably Boc-protected) N-terminus.

In some embodiments, the C-terminus of the protected variant of Peptide B is amidated, and the N-terminus of the protected variant of Peptide D is protected (preferably Boc-protected).

In preferred embodiments, the protected variant of Peptide B has a free amino group at the N-terminus and does not have a free carboxyl group at the C-terminus, and the protected variant of Peptide D has a free carboxyl group at the C-terminus and does not have a free amino group at the N-terminus. In particularly preferred embodiments, the protected variant of Peptide B has a free amino group at the N-terminus and an amidated C-terminus, and the protected variant of Peptide D has a free carboxyl group at the C-terminus and a protected (preferably Boc-protected) N-terminus.

In some embodiments, the side chain of the Trp residue at position 2, the side chain of the Lys residue at position 5, the side chain of the Ser residue at position 13, the side chain of the Ser residue at position 18, the side chain of the Ser residue at position 23, and the side chain of the Lys residue at position 24 of the protected variant of Peptide B are protected, the protected variant of Peptide B has a free amino group at the N-terminus, and the protected variant of Peptide B does not have a free carboxyl group at the C-terminus.

In some embodiments, the N-terminus, the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, the side chain of the Ser residue at position 12, the side chain of the Tyr residue at position 13, the side chain of the Glu residue at position 15, the side chain of the Glu residue at position 16, the side chain of the Gln residue at position 17, the side chain of the Lys residue at position 20, and the side chain of the Glu residue at position 21 of the protected variant of Peptide D are protected and the protected variant of Peptide D has a free carboxyl group at the C-terminus.

In some embodiments, the side chain of the Trp residue at position 2, the side chain of the Lys residue at position 5, the side chain of the Ser residue at position 13, the side chain of the Ser residue at position 18, the side chain of the Ser residue at position 23, and the side chain of the Lys residue at position 24 of the protected variant of Peptide B are protected, the protected variant of Peptide B has a free amino group at the N-terminus, the protected variant of Peptide B does not have a free carboxyl group at the C-terminus, the N-terminus, the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, the side chain of the Ser residue at position 12, the side chain of the Tyr residue at position 13, the side chain of the Glu residue at position 15, the side chain of the Glu residue at position 16, the side chain of the Gln residue at position 17, the side chain of the Lys residue at position 20, and the side chain of the Glu residue at position 21 of the protected variant of Peptide D are protected, and the protected variant of Peptide D has a free carboxyl group at the C-terminus.

In some embodiments, the side chain of the Trp residue at position 2 and the side chain of the Lys residue at position 5 of the protected variant of Peptide B are each protected with a tert-butyloxycarbonyl (Boc) protecting group, the Ser residues at position 13, position 18, and position 23 of the protected variant of Peptide B are each protected as a dimethylated pseudoproline (Psi(Me, Me) pro) moiety in which the oxazolidine is derived from Ser, and the side chain of the Lys residue at position 24 of the protected variant of Peptide B is protected with an ivDde (1-(4,4-dimethyl-2,6-dioxocyclohex-1-ylidene)-3-methylbutyl) protecting group.

In some embodiments, the side chain of the His residue at position 1 of Peptide D is protected with a trityl (Trt) protecting group; the side chains of the Glu residues at position 3, position 15, position 16, and position 21 of Peptide D are each protected with a tert-butyl ester (OtBu) protecting group; the side chains of the Thr residues at position 5 and position 7 of Peptide D are each protected with a tert-butyl (tBu) protecting group; the side chains of the Ser residues at position 8 and position 11 of Peptide D are each protected with a tBu protecting group; the side chain of the Asp residue at position 9 of Peptide D is protected with an OtBu protecting group; the side chains of the Tyr residues at positions 10 and 13 of Peptide D are each protected with a tBu protecting group; the Ser residue at position 12 of Peptide D is protected as a dimethylated pseudoproline (Psi(Me, Me) pro) moiety in which the oxazolidine is derived from Ser; the side chain of the Gln residue at position 17 of Peptide D is protected with a Trt protecting group; and the side chain of the Lys residue at position 20 of Peptide D is protected with a tert-butyloxycarbonyl (Boc) protecting group.

In some embodiments, the side chain of the Trp residue at position 2 and the side chain of the Lys residue at position 5 of the protected variant of Peptide B are each protected with a tert-butyloxycarbonyl (Boc) protecting group, the Ser residues at position 13, position 18, and position 23 of the protected variant of Peptide B are each protected as a dimethylated pseudoproline (Psi(Me, Me) pro) moiety in which the oxazolidine is derived from Ser, the side chain of the Lys residue at position 24 of the protected variant of Peptide B is protected with an ivDde (1-(4,4-dimethyl-2,6-dioxocyclohex-1-ylidene)-3-methylbutyl) protecting group, the side chain of the His residue at position 1 of Peptide D is protected with a trityl (Trt) protecting group; the side chains of the Glu residues at position 3, position 15, position 16, and position 21 of Peptide D are each protected with a tert-butyl ester (OtBu) protecting group; the side chains of the Thr residues at position 5 and position 7 of Peptide D are each protected with a tert-butyl (tBu) protecting group; the side chains of the Ser residues at position 8 and position 11 of Peptide D are each protected with a tBu protecting group; the side chain of the Asp residue at position 9 of Peptide D is protected with an OtBu protecting group; the side chains of the Tyr residues at positions 10 and 13 of Peptide D are each protected with a tBu protecting group; the Ser residue at position 12 of Peptide D is protected as a dimethylated pseudoproline (Psi(Me, Me) pro) moiety in which the oxazolidine is derived from Ser; the side chain of the Gln residue at position 17 of Peptide D is protected with a Trt protecting group; and the side chain of the Lys residue at position 20 of Peptide D is protected with a tert-butyloxycarbonyl (Boc) protecting group.

In some embodiments, the protected variant of Peptide B is Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 147).

In some embodiments, the protected variant of Peptide D is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 157) or Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 168). In some embodiments, the protected variant of Peptide D is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 157). In other embodiments, the protected variant of Peptide D is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 168).

In some embodiments, the protected variant of Peptide B is Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 147), and the protected variant of Peptide D is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 157) or Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 168).

In some embodiments, the protected variant of Peptide B is Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me, Me)Pro)-Gly-Gly-Gly-Gly-Ser(Psi(Me,Me)Pro)-Lys(ivDde)-NH₂ (SEQ ID NO: 147), and the protected variant of Peptide D is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 157).

In some embodiments, the protected variant of Peptide B is Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 147), and the protected variant of Peptide D is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 168).

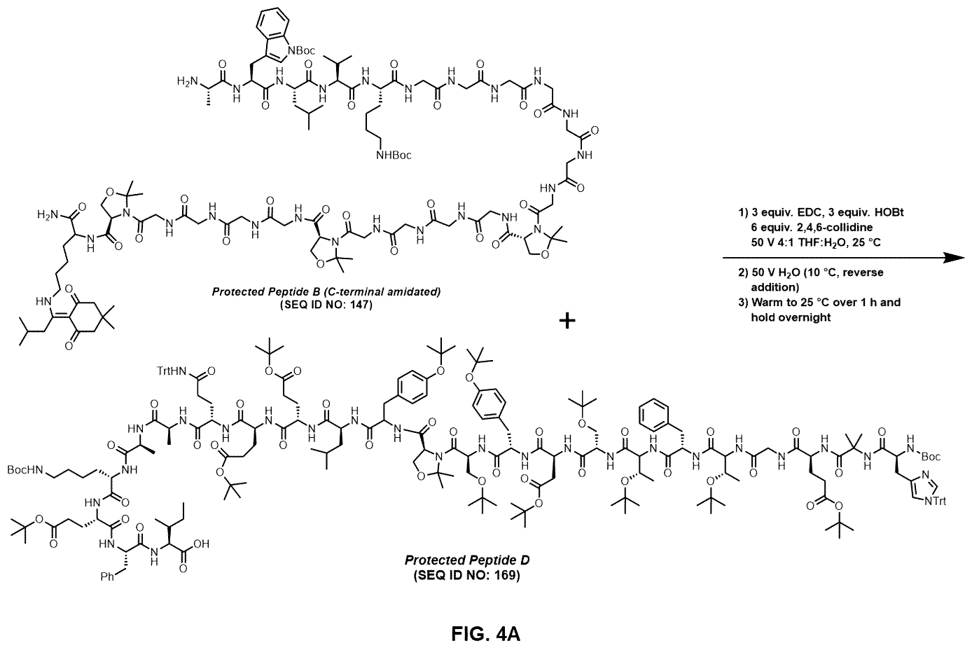

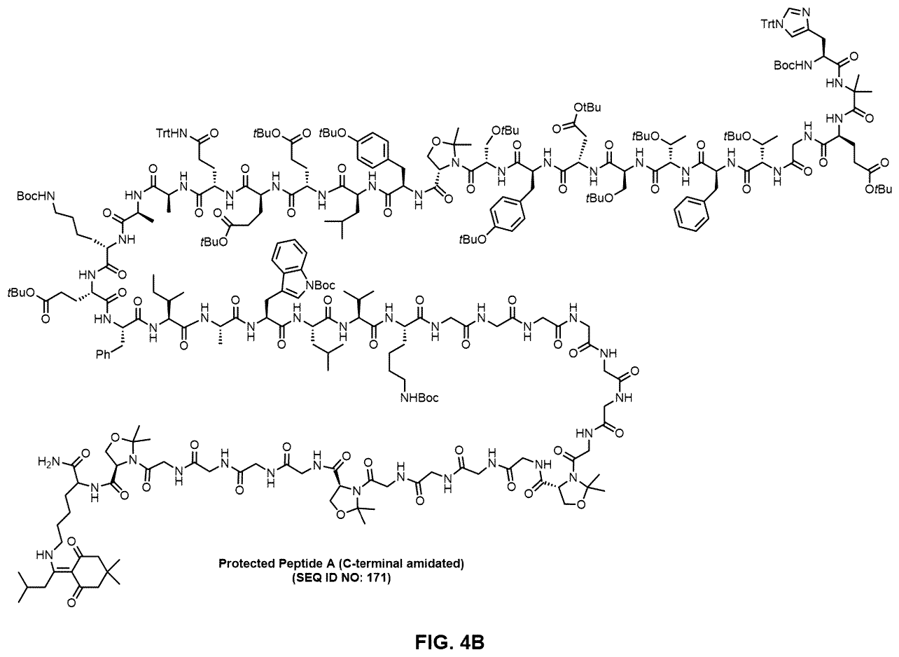

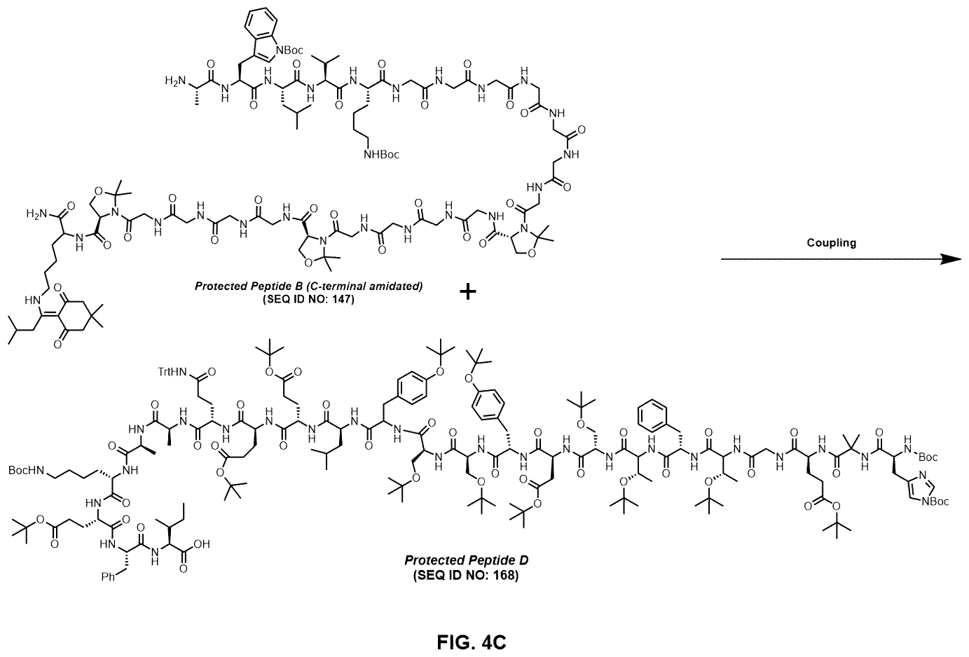

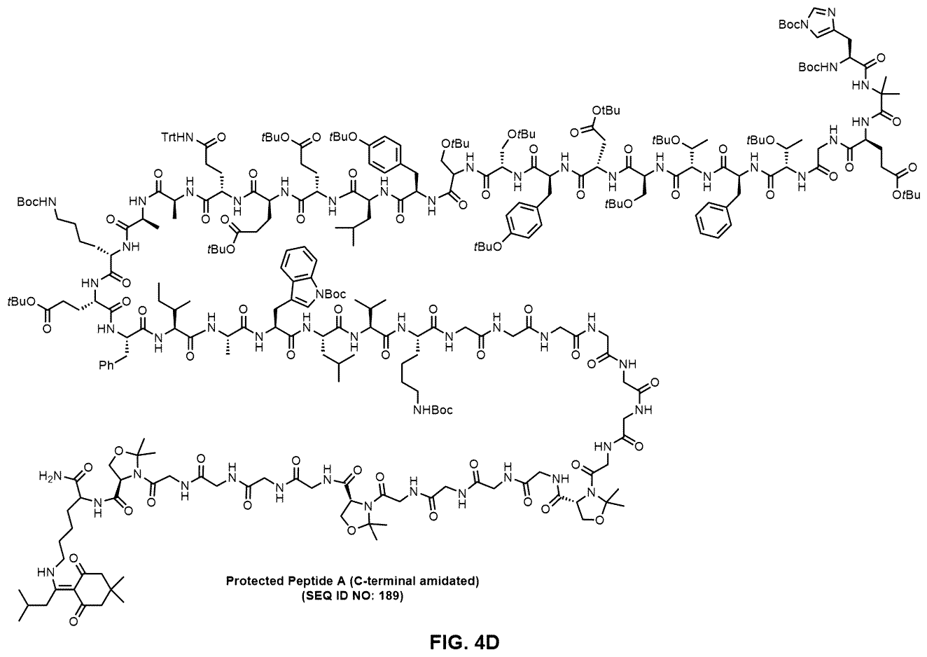

In some embodiments, a protected variant of Peptide B with an amidated C-terminus and a protected variant of Peptide D can be coupled in solution to form a protected variant of Peptide A with an amidated C-terminus as shown in FIGS. 4A and 4B.

In some embodiments, the coupling composition comprises 1 to 10 equivalents of a coupling reagent, optionally 1 to 10 equivalents of a base, and optionally 1 to 10 equivalents of a coupling additive, wherein equivalents are relative to the protected variant of Peptide D. In some embodiments, the coupling composition comprises 1 to 5 equivalents of a coupling reagent, optionally 1 to 10 equivalents of a base, and optionally 1 to 5 equivalents of a coupling additive, wherein equivalents are relative to the protected variant of Peptide D.

In some embodiments, the coupling composition comprises DIC/Oxyma, T3P/Oxyma, T3P/Oxyma/collidine, EDC/HOBt/collidine, HCTU/collidine, HATU/collidine, HBTU/collidine, TBTU/collidine, DMAP/EDC, Oxyma/PyOxim, Oxyma/PyOxim/collidine, PyBOP/HOBt/collidine, or HCTU/6-Cl—HOBt/collidine.

In some embodiments, the coupling composition comprises HCTU and collidine.

In some embodiments, the coupling composition comprises HATU and collidine.

In some embodiments, the coupling composition comprises EDC, HOBt, and collidine. In some embodiments, the coupling composition comprises EDC, HOBt, and collidine in THF and water. In some embodiments, the coupling composition comprises EDC, HOBt, and collidine in 4:1 THF:water.

In some embodiments, the coupling composition comprises PyBOP, HOBt, and collidine. In some embodiments, the coupling composition comprises PyBOP, HOBt, and collidine in THF and acetonitrile. In some embodiments, the coupling composition comprises PyBOP, HOBt, and collidine in 1:1 THF:acetonitrile.

In some embodiments, the coupling composition comprises PyOxim, Oxyma, and collidine. In some embodiments, the coupling composition comprises PyOxim, Oxyma, and collidine in THF and acetonitrile. In some embodiments, the coupling composition comprises PyOxim, Oxyma, and collidine in 1:1 THF:acetonitrile.

In some embodiments, the coupling composition comprises HCTU, 6-Cl—HOBt, and collidine. In some embodiments, the coupling composition comprises HCTU, 6-Cl—HOBt, and collidine in a solvent selected from DMF, acetonitrile, THF, 2-MeTHF, and combinations of any of the foregoing. In some embodiments, the coupling composition comprises HCTU, 6-Cl—HOBt, and collidine in acetonitrile.

In some embodiments, the coupling composition comprises 1 to 10 equivalents of EDC, 1 to 10 equivalents of collidine, and 1 to 10 equivalents of HOBt in THF and water, wherein equivalents are relative to the protected variant of Peptide D. In some embodiments, the coupling composition comprises 1 to 5 equivalents of EDC, 1 to 10 equivalents of collidine, and 1 to 5 equivalents of HOBt in THE and water, wherein equivalents are relative to the protected variant of Peptide D.

In some embodiments, the coupling composition comprises 1 to 10 equivalents of EDC, 1 to 10 equivalents of collidine, and 1 to 10 equivalents of HOBt in 3-5:1 THF:water (e.g., 4:1 THF:water), wherein equivalents are relative to the protected variant of Peptide D. In some embodiments, the coupling composition comprises 1 to 5 equivalents of EDC, 1 to 10 equivalents of collidine, and 1 to 5 equivalents of HOBt in 3-5:1 THF:water (e.g., 4:1 THF:water), wherein equivalents are relative to the protected variant of Peptide D.

In some embodiments, the coupling occurs at a temperature in the range of 0° C. to 60° C. In some embodiments, the coupling occurs at a temperature in the range of 20° C. to 50° C. In some embodiments, the coupling occurs at a temperature in the range of 20° C. to 30° C. In some embodiments, the coupling occurs at a temperature in the range of 30° C. to 50° C. In some embodiments, the coupling occurs at a temperature in the range of 35° C. to 45° C.

In some embodiments, the method comprises coupling the protected variant of Peptide B and the protected variant of Peptide D for a time in the range of 1 hour to 72 hours. In some embodiments, the time is in the range of 6 hours to 24 hours.

In some embodiments, the coupling occurs at a temperature in the range of 0° C. to 60° C. for a time in the range of 1 hour to 72 hours. In some embodiments, the coupling occurs at a temperature in the range of 0° C. to 60° C. for a time in the range of 6 hours to 24 hours. In some embodiments, the coupling occurs at a temperature in the range of 20° C. to 50° C. for a time in the range of 1 hour to 72 hours. In some embodiments, the coupling occurs at a temperature in the range of 20° C. to 50° C. for a time in the range of 6 hours to 24 hours. In some embodiments, the coupling occurs at a temperature in the range of 20° C. to 30° C. for a time in the range of 1 hour to 72 hours. In some embodiments, the coupling occurs at a temperature in the range of 20° C. to 30° C. for a time in the range of 6 hours to 24 hours.

In some embodiments, the coupling composition comprises 1 to 10 equivalents of EDC, 1 to 10 equivalents of collidine, and 1 to 10 equivalents of HOBt in 3-5:1 THF:water (e.g., 4:1 THF:water), wherein equivalents are relative to the protected variant of Peptide D, and the coupling occurs at a temperature in the range of 20° C. to 30° C. for a time in the range of 6 hours to 24 hours. In some embodiments, the coupling composition comprises 1 to 5 equivalents of EDC, 1 to 10 equivalents of collidine, and 1 to 5 equivalents of HOBt in 3-5:1 THF:water (e.g., 4:1 THF:water), wherein equivalents are relative to the protected variant of Peptide D, and the coupling occurs at a temperature in the range of 20° C. to 30° C. for a time in the range of 6 hours to 24 hours.

In some embodiments, the protected variant of Peptide B is Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me, Me)Pro)-Gly-Gly-Gly-Gly-Ser(Psi(Me,Me)Pro)-Lys(ivDde)-NH₂ (SEQ ID NO: 147), the protected variant of Peptide D is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 157) or Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 168), the coupling composition comprises DIC/Oxyma, T3P/Oxyma, T3P/Oxyma/collidine, EDC/HOBt/collidine, HCTU/collidine, HATU/collidine, HBTU/collidine, TBTU/collidine, DMAP/EDC, Oxyma/PyOxim, Oxyma/PyOxim/collidine, PyBOP/HOBt/collidine, or HCTU/6-Cl—HOBt/collidine (preferably HCTU/6-Cl—HOBt/collidine or EDC/HOBt/collidine), and the coupling occurs at a temperature in the range of 0° C. to 60° C. (preferably a temperature in the range of 20° C. to 50° C., more preferably a temperature in the range of 20° C. to 30° C.) for a time in the range of 1 hour to 72 hours (preferably a time in the range of 6 hours to 24 hours).

In some embodiments, the protected variant of Peptide B is Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 140), the protected variant of Peptide D is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 168), and the coupling composition comprises HCTU, 6-Cl—HOBt, and collidine. In some embodiments, the protected variant of Peptide B and the protected variant of Peptide D are contacted at room temperature. In some embodiments, the protected variant of Peptide B and the protected variant of Peptide D are contacted at room temperature for a time in the range of 6 hours to 24 hours.

In some embodiments, the protected variant of Peptide B is Ala-Trp(Boc)-Leu-Val-Lys(Boc)-Gly-Gly-Gly-Gly-Gly-Gly-Gly-Ser(Psi(Me, Me)Pro)-Gly-Gly-Gly-Gly-Ser(Psi(Me, Me)Pro)-Gly-Gly-Gly-Gly-Ser(Psi(Me,Me)Pro)-Lys(ivDde)-NH₂ (SEQ ID NO: 147), the protected variant of Peptide D is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 157), and the coupling composition comprises EDC, HOBt, and collidine. In some embodiments, the protected variant of Peptide B and the protected variant of Peptide D are contacted at room temperature for a time in the range of 6 hours to 24 hours.

In some embodiments, the protected variant of Peptide B and/or the protected variant of Peptide D is prepared by a method provided herein.

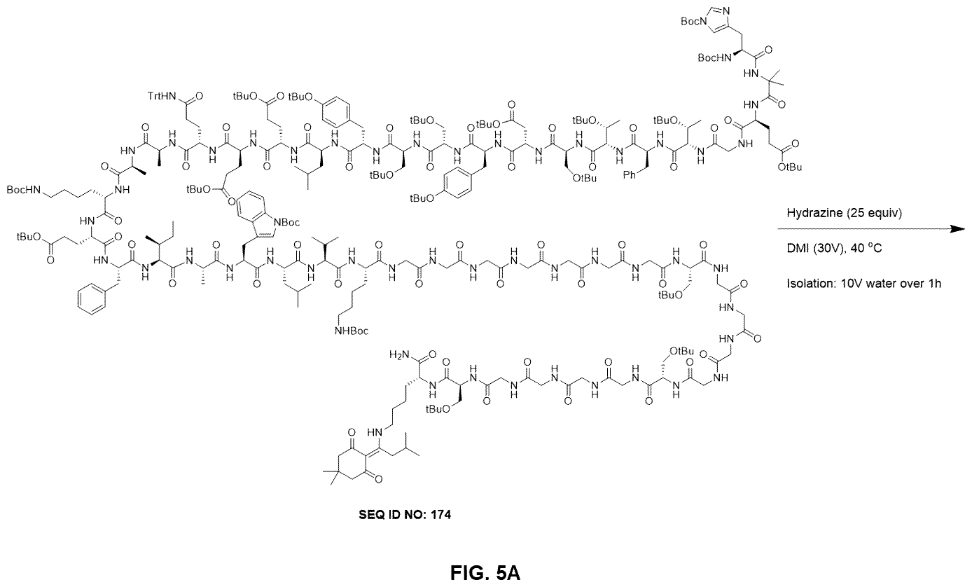

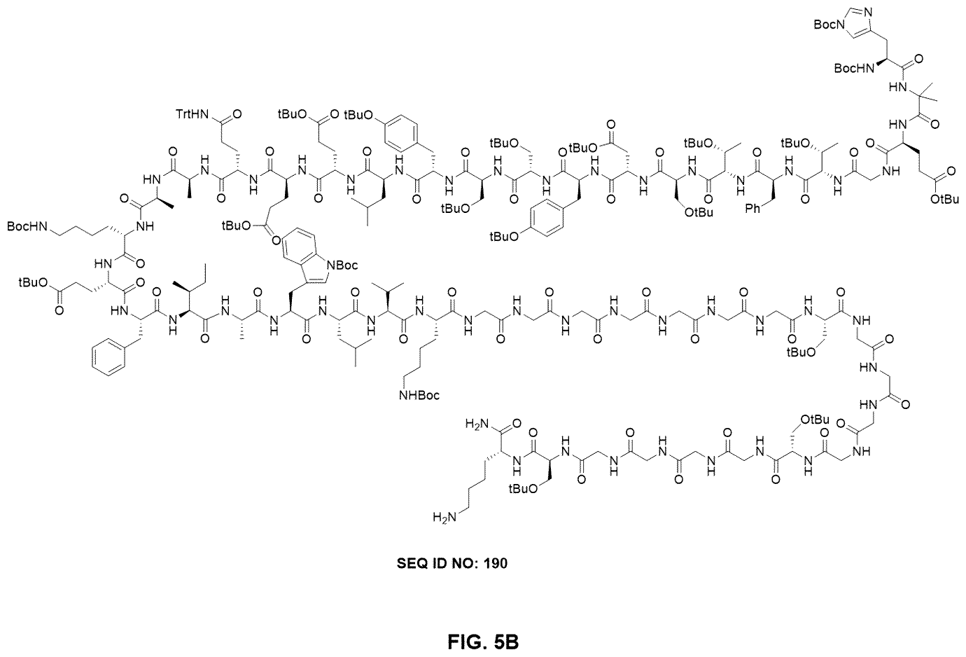

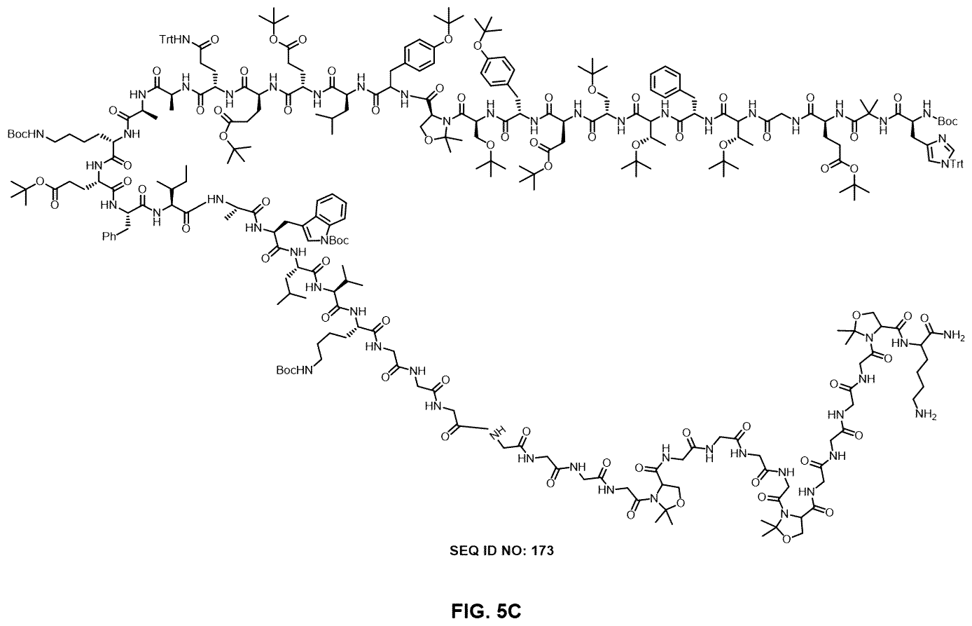

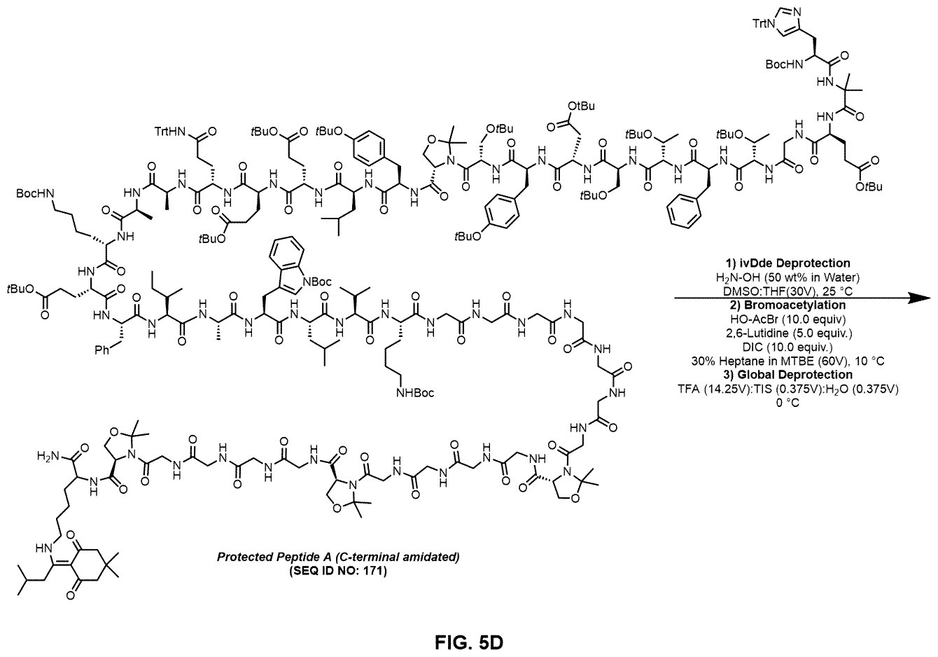

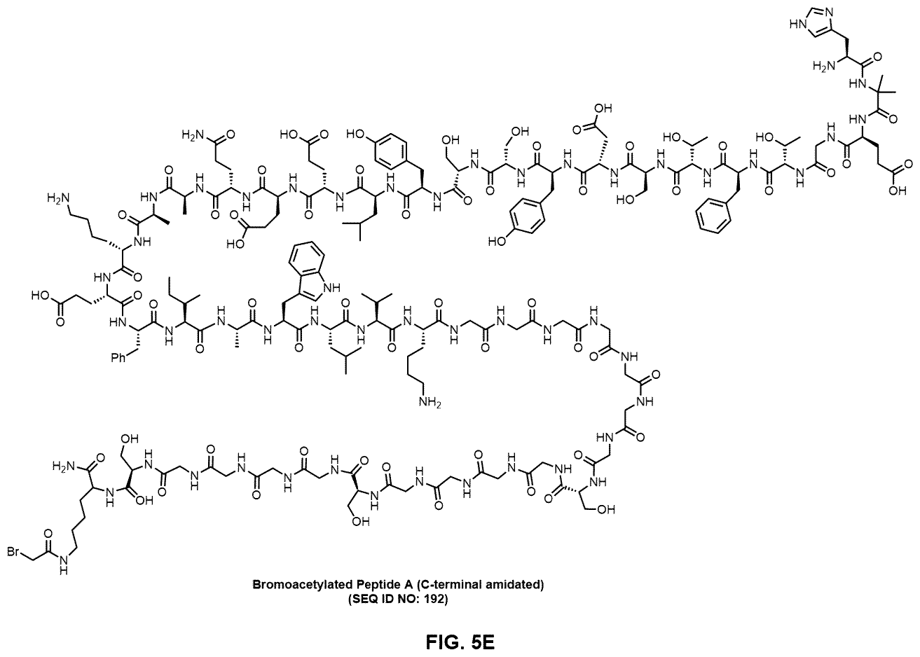

Additionally, in certain embodiments, a protected variant of Peptide A can be partially deprotected as illustrated in FIG. 5A to form a different protected variant of Peptide A (FIG. 5B) in which the protecting group on the &-amino group of the lysine residue at the C-terminus has been removed. FIG. 5C depicts another partially deprotected variant of Peptide A which can be prepared by an analogous process to that shown in FIG. 5A.

In some embodiments, the side chain of the Lys residue at the C-terminus of the protected variant of Peptide A formed by coupling the protected variant of Peptide B with the protected variant of Peptide D is protected with a protecting group that can be selectively removed (such as, e.g., a 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), benzyloxycarbonyl (Cbz), or chlorobenzyloxycarbonyl(2-Cl—Z) protecting group) and the method further comprises contacting the protected variant of Peptide A with a selective deprotection reagent to remove the protecting group and form a partially deprotected variant of Peptide A.

In some embodiments, the side chain of the Lys residue at the C-terminus of the protected variant of Peptide A formed by coupling the protected variant of Peptide B with the protected variant of Peptide D is protected with a 1-(4,4-dimethyl-2,6-dioxocyclohex-1-ylidene)-3-methylbutyl (ivDde) protecting group, and the method further comprises contacting the protected variant of Peptide A with an α-nucleophile to remove the ivDde protecting group and form a partially deprotected variant of Peptide A. In some embodiments, the α-nucleophile is hydrazine or hydroxylamine, preferably hydrazine. In some embodiments, the α-nucleophile is hydrazine, and the contacting occurs in a solvent selected from DMF, DMSO, DMI, acetonitrile, methanol, THF, and combinations of any of the foregoing. In some embodiments, the α-nucleophile is hydroxylamine, and the contacting occurs in a solvent selected from acetonitrile, DMF, THF, DMSO, NMP, DMI, methanol, water, and combinations of any of the foregoing.

In some embodiments, the contacting occurs at a temperature in the range of 0° C. to 60° C. In some embodiments, the contacting occurs at a temperature in the range of 20° C. to 50° C. In some embodiments, the contacting occurs at a temperature in the range of 20° C. to 30° C.

In some embodiments, the side chain of the Lys residue at the C-terminus of the protected variant of Peptide A formed by coupling the protected variant of Peptide B with the protected variant of Peptide D is protected with a 1-(4,4-dimethyl-2,6-dioxocyclohex-1-ylidene)-3-methylbutyl (ivDde) protecting group, and the method further comprises contacting the protected variant of Peptide A with hydrazine (e.g., hydrazine in DMI) at a temperature in the range of 20° C. to 30° C. to remove the ivDde protecting group and form a partially deprotected variant of Peptide A.

In some embodiments, the side chain of the Lys residue at the C-terminus of the protected variant of Peptide A formed by coupling the protected variant of Peptide B with the protected variant of Peptide D is protected with a 1-(4,4-dimethyl-2,6-dioxocyclohex-1-ylidene)-3-methylbutyl (ivDde) protecting group, and the method further comprises contacting the protected variant of Peptide A with hydroxylamine (e.g., hydroxylamine in DMSO and THF) at a temperature in the range of 20° C. to 30° C. to remove the ivDde protecting group and form a partially deprotected variant of Peptide A.

In some embodiments, the method further comprises contacting the partially deprotected variant of Peptide A with bromoacetic acid or bromoacetic anhydride in the presence of a second coupling composition to form a bromoacetylated protected variant of Peptide A. In some embodiments, the second coupling composition comprises a coupling reagent, a base, and optionally a coupling additive.

In some embodiments, the method further comprises contacting the partially deprotected variant of Peptide A with bromoacetic acid in the presence of a second coupling composition to form a bromoacetylated protected variant of Peptide A, wherein the second coupling composition comprises TBTU and collidine. In some embodiments, the second coupling composition further comprises N-hydroxysuccinimide ester (NHS).

In some embodiments, the method further comprises contacting the partially deprotected variant of Peptide A with bromoacetic acid in the presence of a second coupling composition to form a bromoacetylated protected variant of Peptide A, wherein the second coupling composition comprises DIC and 2,6-lutidine. In some embodiments, the second coupling composition comprises DIC and 2,6-lutidine in heptane and MTBE.

In some embodiments, the method further comprises contacting the partially deprotected variant of Peptide A with bromoacetic anhydride in the presence of a second coupling composition to form a bromoacetylated protected variant of Peptide A, wherein the second coupling composition comprises lithium bromide and N-methylmorpholine. In some embodiments, the second coupling composition comprises lithium bromide and N-methylmorpholine in THF.

In some embodiments, the method further comprises deprotecting the bromoacetylated protected variant of Peptide A by contacting the bromoacetylated protected variant of Peptide A with a Brønsted acid or a Lewis acid (preferably trifluoroacetic acid (TFA)), optionally in the presence of a scavenger (preferably triisopropylsilane (TIS)). In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some embodiments, the method further comprises deprotecting the bromoacetylated protected variant of Peptide A by contacting the bromoacetylated protected variant of Peptide A with an aqueous solution comprising TFA and TIS.

In some embodiments, the method further comprises globally deprotecting the bromoacetylated protected variant of Peptide A.

In some embodiments in which the N-terminus of the bromoacetylated protected variant of Peptide A is Boc-protected, the method further comprises deprotecting the bromoacetylated protected variant of Peptide A by contacting the bromoacetylated protected variant of Peptide A with a Brønsted acid or a Lewis acid (preferably trifluoroacetic acid (TFA)), optionally in the presence of a scavenger (preferably triisopropylsilane (TIS)). In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some embodiments in which the N-terminus of the bromoacetylated protected variant of Peptide A is Boc-protected, the method further comprises deprotecting the bromoacetylated protected variant of Peptide A by contacting the bromoacetylated protected variant of Peptide A with an aqueous solution comprising TFA and TIS.

Alternatively, a protected variant of Peptide A can be partially deprotected to form a protected variant of Peptide A in which the protecting group on the s-amino group of the lysine residue at the C-terminus has been removed; the ε-amino group of the lysine residue at the C-terminus can then be bromoacetylated and the protected peptide globally deprotected as shown in FIG. 5D to form the product shown in FIG. 5E.

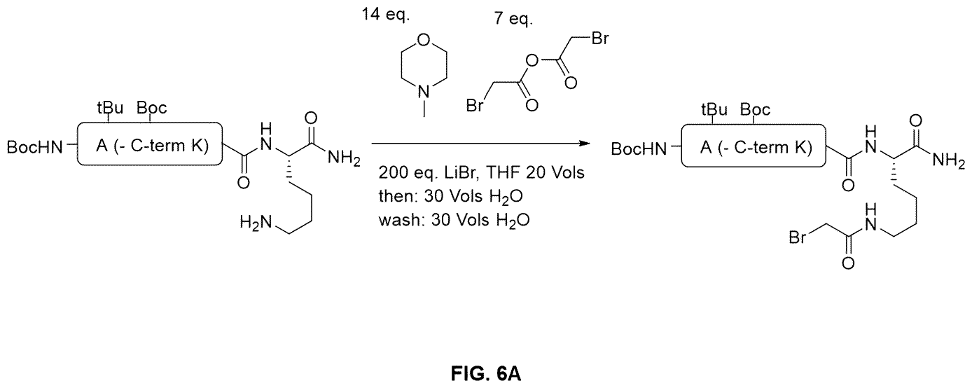

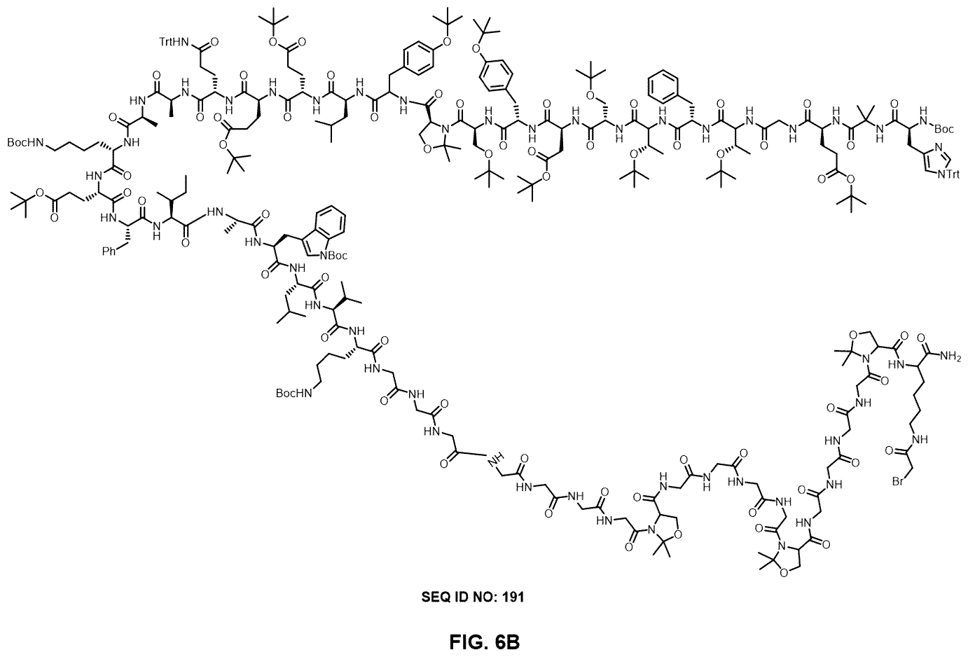

In alternative embodiments, the C-terminal lysine residue of a protected variant of Peptide A can be bromoacetylated as illustrated in FIG. 6A. FIG. 6B depicts a bromoacetylated and protected variant of Peptide A with an amidated C-terminus which can be prepared by the process shown in FIG. 6A.

Also provided herein is a method of preparing Peptide A or a protected variant thereof, comprising:
(A)
(i) (a) coupling or having coupled a protected variant of Fragment 3 with a protected variant of Fragment 4 to form a first protected variant of Peptide E, wherein the protected variant of Fragment 3 has a free carboxyl group at the C-terminus and a protected (preferably Fmoc-protected) N-terminus, the protected variant of Fragment 4 has a free amino group at the N-terminus, Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8; (b) selectively deprotecting or having selectively deprotected the N-terminus of the first protected variant of Peptide E from step (A) (i) (a) to form a second protected variant of Peptide E with a free amino group at the N-terminus; (c) coupling or having coupled the second protected variant of Peptide E from step (A) (i) (b) with an N-protected alanine to form a first protected variant of Peptide B, wherein Peptide B comprises the amino acid sequence of SEQ ID NO: 9; and (d) selectively deprotecting or having selectively deprotected the N-terminus of the first protected variant of Peptide B from step (A) (i) (c) to form a second protected variant of Peptide B with a free amino group at the N-terminus; OR
(ii) (a) coupling or having coupled a protected variant of Fragment 3A with a protected variant of Fragment 4 to form a first protected variant of Peptide B, wherein the protected variant of Fragment 3A has a free carboxyl group at the C-terminus and a protected (preferably Fmoc-protected) N-terminus, the protected variant of Fragment 4 has a free amino group at the N-terminus, Fragment 3A comprises the amino acid sequence of SEQ ID NO: 13, Fragment 4 comprises the amino acid sequence of SEQ ID NO: 4, and Peptide B comprises the amino acid sequence of SEQ ID NO: 9; and (b) selectively deprotecting or having selectively deprotected the N-terminus of the first protected variant of Peptide B from step (A) (ii) (a) to form a second protected variant of Peptide B with a free amino group at the N-terminus; OR
(iii) (a) coupling or having coupled a protected variant of Fragment 5 with a protected variant of Fragment 6 to form a first protected variant of Peptide B, wherein the protected variant of Fragment 5 has a free carboxyl group at the C-terminus and a protected (preferably Trt- or Fmoc-protected, more preferably Trt-protected) N-terminus, the protected variant of Fragment 6 has a free amino group at the N-terminus, Fragment 5 comprises the amino acid sequence of SEQ ID NO: 5, Fragment 6 comprises the amino acid sequence of SEQ ID NO: 6, and Peptide B comprises the amino acid sequence of SEQ ID NO: 9; and (b) selectively deprotecting or having selectively deprotected the N-terminus of the first protected variant of Peptide B from step (A) (iii) (a) to form a second protected variant of Peptide B with a free amino group at the N-terminus;
(B) coupling or having coupled a protected variant of Fragment 1 with a protected variant of Fragment 2B to form a protected variant of Peptide D, wherein the protected variant of Fragment 1 has a free carboxyl group at the C-terminus and a protected (preferably Boc-protected) N-terminus, the protected variant of Fragment 2B has a free amino group at the N-terminus, Fragment 1 comprises the amino acid sequence of SEQ ID NO: 1, Fragment 2B comprises the amino acid sequence of SEQ ID NO: 16, and Peptide D comprises the amino acid sequence of SEQ ID NO: 15; and
(C) coupling or having coupled the second protected variant of Peptide B from step (A) with the protected variant of Peptide D from step (B) to form a protected variant of Peptide A, wherein Peptide A comprises the amino acid sequence of SEQ ID NO: 7.

In some embodiments, the method further comprises deprotecting or having deprotected the protected variant of Peptide A to isolate Peptide A.

In some embodiments, the method further comprises adding or having added a bromoacetyl moiety to the Lys at the C-terminus of Peptide A or a protected variant thereof.

In some embodiments, the method comprises step (A) (i), step (B), and step (C). In other embodiments, the method comprises step (A) (ii), step (B), and step (C). In other preferred embodiments, the method comprises step (A) (iii), step (B), and step (C).

In some embodiments, the method comprises step (A) (i), step (B), and step (C), wherein each coupling is performed under liquid phase peptide coupling conditions. In other embodiments, the method comprises step (A) (ii), step (B), and step (C), wherein each coupling is performed under liquid phase peptide coupling conditions. In other preferred embodiments, the method comprises step (A) (iii), step (B), and step (C), wherein each coupling is performed under liquid phase peptide coupling conditions.

In some embodiments, the method comprises step (A) (i), step (B), and step (C), wherein each coupling is performed under solid phase peptide coupling conditions. In other embodiments, the method comprises step (A) (ii), step (B), and step (C), wherein each coupling is performed under solid phase peptide coupling conditions. In other embodiments, the method comprises step (A) (iii), step (B), and step (C), wherein each coupling is performed under solid phase peptide coupling conditions.

In some embodiments, the protected variant of Fragment 3 and the protected variant of Fragment 4 are coupled in step (A) (i) (a) under conditions described herein.

In some embodiments, the second protected variant of Peptide E and the N-protected alanine are coupled in step (A) (i) (c) under conditions described herein.

In some embodiments, the protected variant of Fragment 3A and the protected variant of Fragment 4 are coupled in step (A) (ii) (a) are coupled under conditions described herein.

In some embodiments, the protected variant of Fragment 5 and the protected variant of Fragment 6 are coupled in step (A) (iii) (a) are coupled under conditions described herein.

In some embodiments, the protected variant of Fragment 1 and the protected variant of Fragment 2B are coupled in step (B) are coupled under conditions described herein.

In some embodiments, the second protected variant of Peptide B and the protected variant of Peptide D are coupled in step (C) are coupled under conditions described herein.

In some embodiments, the method comprises step (A) (i), the N-terminus of the first protected variant of Peptide E is Fmoc-protected, and the selective deprotection of step (A) (i) (b) comprises contacting the first protected variant of Peptide E with an amine base to selectively remove the Fmoc protecting group at the N-terminus. In some embodiments, the amine base is selected from piperidine, piperazine, and morpholine (e.g., 5-30% (v/v) piperidine, piperazine, or morpholine). In some embodiments, the amine base is piperidine. In some embodiments, the method comprises step (A) (i), the N-terminus of the first protected variant of Peptide E is Fmoc-protected, and the selective deprotection of step (A) (i) (b) comprises contacting the first protected variant of Peptide E with piperidine in DMF, optionally in the presence of DBU.

In some embodiments, the method comprises step (A) (i), the N-terminus of the first protected variant of Peptide E is Boc-protected, and the selective deprotection of step (A) (i) (b) comprises contacting the first protected variant of Peptide E with an acid (preferably an acid selected from TFA, HCl, and HBr) to selectively remove the Boc protecting group at the N-terminus. In some embodiments, the first protected variant of Peptide E is contacted with TFA (e.g., neat TFA or TFA in DCM, e.g., 20-50% (v/v) TFA in DCM). In some embodiments, the first protected variant of Peptide E is contacted with HCl (e.g., HCl in DCM, DCE, toluene, or dioxane, e.g., 3-4 M HCl in DCM or dioxane).

In some embodiments, the method comprises step (A) (i), the N-terminus of the first protected variant of Peptide E is Boc-protected, and the selective deprotection of step (A) (i) (b) comprises contacting the first protected variant of Peptide E with an acid (preferably an acid selected from TFA, HCl, and HBr) in the presence of a scavenger. In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some preferred embodiments, the scavenger is a hydrosilane. In some particularly preferred embodiments, the scavenger is TIS. In some embodiments, the first protected variant of Peptide E is contacted with TFA (e.g., neat TFA or TFA in DCM, e.g., 20-50% (v/v) TFA in DCM) in the presence of a scavenger (preferably TIS). In some embodiments, the first protected variant of Peptide E is contacted with HCl (e.g., HCl in DCM, DCE, toluene, or dioxane, e.g., 3-4 M HCl in DCM or dioxane) in the presence of a scavenger.

In some embodiments, the method comprises step (A) (i), the N-terminus of the first protected variant of Peptide E is Trt-protected, and the selective deprotection of step (A) (i) (b) comprises contacting the first protected variant of Peptide E with a Brønsted acid or a Lewis acid (preferably TFA, HCl, or dichloroacetic acid, most preferably TFA), optionally in the presence of a scavenger (preferably TIS), to selectively remove the trityl protecting group at the N-terminus. In some embodiments, the first protected variant of Peptide E is contacted with a Brønsted acid (preferably a dilute Brønsted acid). In some embodiments, the Brønsted acid is selected from TFA, HCl, and dichloroacetic acid (preferably dilute TFA, HCl, or dichloroacetic acid). In other embodiments, the first protected variant of Peptide E is contacted with a Lewis acid (preferably a dilute Lewis acid).

In some embodiments, the method comprises step (A) (i), the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some embodiments, the scavenger is a hydrosilane. In some embodiments, the scavenger is TIS.

In some embodiments, the method comprises step (A) (i), the N-terminus of the first protected variant of Peptide E is Trt-protected, and the selective deprotection of step (A) (i) (b) comprises contacting the first protected variant of Peptide E with HCl. In some embodiments, the selective deprotection of step (A) (i) (b) comprises contacting the first protected variant of Peptide E with dilute HCl.

In some embodiments, the method comprises step (A) (i), the N-terminus of the first protected variant of Peptide E is Trt-protected, and the selective deprotection of step (A) (i) (b) comprises contacting the first protected variant of Peptide E with TFA in the presence of TIS. In some embodiments, the selective deprotection of step (A) (i) (b) comprises contacting the first protected variant of Peptide E with dilute TFA in the presence of TIS.

In some embodiments, the N-terminus of the first protected variant of Peptide B is Fmoc-protected, and the selective deprotection of step (A) (i) (d), step (A) (ii) (b), or step (A) (iii) (b) comprises contacting the first protected variant of Peptide B with an amine base to selectively remove the Fmoc protecting group at the N-terminus. In some embodiments, the amine base is selected from piperidine, piperazine, and morpholine (e.g., 5-30% (v/v) piperidine, piperazine, or morpholine). In some embodiments, the amine base is piperidine. In some embodiments, the N-terminus of the first protected variant of Peptide B is Fmoc-protected, and the selective deprotection of step (A) (i) (d), step (A) (ii) (b), or step (A) (iii) (b) comprises contacting the first protected variant of Peptide B with piperidine in DMF, optionally in the presence of DBU.

In some embodiments, the N-terminus of the first protected variant of Peptide B is Boc-protected, and the selective deprotection of step (A) (i) (d), step (A) (ii) (b), or step (A) (iii) (b) comprises contacting the first protected variant of Peptide B with an acid (preferably an acid selected from TFA, HCl, and HBr) to selectively remove the Boc protecting group at the N-terminus. In some embodiments, the first protected variant of Peptide B is contacted with TFA (e.g., neat TFA or TFA in DCM, e.g., 20-50% (v/v) TFA in DCM). In some embodiments, the first protected variant of Peptide B is contacted with HCl (e.g., HCl in DCM, DCE, toluene, or dioxane, e.g., 3-4 M HCl in DCM or dioxane).

In some embodiments, the N-terminus of the first protected variant of Peptide B is Boc-protected, and the selective deprotection of step (A) (i) (d), step (A) (ii) (b), or step (A) (iii) (b) comprises contacting the first protected variant of Peptide B with an acid (preferably an acid selected from TFA, HCl, and HBr) in the presence of a scavenger. In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some preferred embodiments, the scavenger is a hydrosilane. In some particularly preferred embodiments, the scavenger is TIS. In some embodiments, the first protected variant of Peptide B is contacted with TFA (e.g., neat TFA or TFA in DCM, e.g., 20-50% (v/v) TFA in DCM) in the presence of a scavenger (preferably TIS). In some embodiments, the first protected variant of Peptide B is contacted with HCl (e.g., HCl in DCM, DCE, toluene, or dioxane, e.g., 3-4 M HCl in DCM or dioxane) in the presence of a scavenger.

In some embodiments, the N-terminus of the first protected variant of Peptide B is Trt-protected, and the selective deprotection of step (A) (i) (d), step (A) (ii) (b), or step (A) (iii) (b) comprises contacting the first protected variant of Peptide B with a Brønsted acid or a Lewis acid (preferably TFA, HCl, or dichloroacetic acid, most preferably TFA), optionally in the presence of a scavenger (preferably TIS), to selectively remove the trityl protecting group at the N-terminus. In some embodiments, the first protected variant of Peptide B is contacted with a Brønsted acid (preferably a dilute Brønsted acid). In some embodiments, the Brønsted acid is selected from TFA, HCl, and dichloroacetic acid (preferably dilute TFA, HCl, or dichloroacetic acid). In other embodiments, the first protected variant of Peptide B is contacted with a Lewis acid (preferably a dilute Lewis acid).

In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some embodiments, the scavenger is a hydrosilane. In some embodiments, the scavenger is TIS.

In some embodiments, the N-terminus of the first protected variant of Peptide B is Trt-protected, and the selective deprotection of step (A) (i) (d), step (A) (ii) (b), or step (A) (iii) (b) comprises contacting the first protected variant of Peptide B with HCl. In some embodiments, the selective deprotection of step (A) (i) (d), step (A) (ii) (b), or step (A) (iii) (b) comprises contacting the first protected variant of Peptide B with dilute HCl.

In some embodiments, the N-terminus of the first protected variant of Peptide B is Trt-protected, and the selective deprotection of step (A) (i) (d), step (A) (ii) (b), or step (A) (iii) (b) comprises contacting the first protected variant of Peptide B with TFA in the presence of TIS. In some embodiments, the selective deprotection of step (A) (i) (d), step (A) (ii) (b), or step (A) (iii) (b) comprises contacting the first protected variant of Peptide B with dilute TFA in the presence of TIS.

Also provided herein is a method of preparing Peptide C or a protected variant thereof, comprising coupling a protected variant of Fragment 2A with a protected variant of Peptide B to form a protected variant of Peptide C, wherein Fragment 2A comprises the amino acid sequence of SEQ ID NO: 12, Peptide B comprises the amino acid sequence of SEQ ID NO: 9, and Peptide C comprises the amino acid sequence of SEQ ID NO: 10.

In some embodiments, the protected variant of Fragment 2A is coupled with the protected variant of Peptide B in solution. In some embodiments, the protected variant of Fragment 2A is coupled with the protected variant of Peptide B under liquid phase peptide coupling conditions. In some embodiments, the protected variant of Fragment 2A is coupled with the protected variant of Peptide B under classical solution phase peptide coupling conditions. In some embodiments, the protected variant of Fragment 2A is coupled with the protected variant of Peptide B under tag-assisted liquid phase peptide coupling conditions.

In other embodiments, the protected variant of Fragment 2A is coupled with the protected variant of Peptide B on solid support. In some embodiments, the protected variant of Fragment 2A is coupled with the protected variant of Peptide B under solid phase peptide coupling conditions.

In some embodiments, a C-terminal carboxyl group of the protected variant of Fragment 2A is pre-activated. In other embodiments, a C-terminal carboxyl group of the protected variant of Fragment 2A is activated in situ.

In some embodiments, the method comprises:
contacting the protected variant of Fragment 2A with a coupling composition to form a protected variant of Fragment 2A with an activated C-terminal carboxyl group (“activated Fragment 2A”); and
contacting the protected variant of Peptide B with the activated Fragment 2A to form the protected variant of Peptide C.

In some embodiments, the method comprises:
contacting the protected variant of Fragment 2A with a coupling composition on solid support to form a protected variant of Fragment 2A with an activated C-terminal carboxyl group (“activated Fragment 2A”); and
contacting the protected variant of Peptide B with the activated Fragment 2A on solid support to form the protected variant of Peptide C.

In some embodiments, the method comprises:
contacting the protected variant of Fragment 2A with a coupling composition in solution to form a protected variant of Fragment 2A with an activated C-terminal carboxyl group (“activated Fragment 2A”); and
contacting the protected variant of Peptide B with the activated Fragment 2A in solution to form the protected variant of Peptide C.

In some embodiments (e.g., certain embodiments in which a C-terminal carboxyl group of the protected variant of Fragment 2A is pre-activated), the method comprises:
contacting the protected variant of Fragment 2A with a coupling composition to form a protected variant of Fragment 2A with an activated C-terminal carboxyl group (“activated Fragment 2A”); and
contacting the protected variant of Peptide B with the activated Fragment 2A to form the protected variant of Peptide C, wherein the contacting does not occur in the presence of a coupling reagent.

In some embodiments, the protected variant of Fragment 2A is present in molar excess relative to the coupling reagent in the coupling composition during the contacting of the protected variant of Fragment 2A and the coupling composition.

In some embodiments (e.g., certain embodiments in which a C-terminal carboxyl group of the protected variant of Fragment 2A is pre-activated), the method comprises:
contacting the protected variant of Fragment 2A with a coupling composition on solid support to form a protected variant of Fragment 2A with an activated C-terminal carboxyl group (“activated Fragment 2A”); and
contacting the protected variant of Peptide B with the activated Fragment 2A on solid support to form the protected variant of Peptide C, wherein the contacting does not occur in the presence of a coupling reagent.

In some embodiments, the protected variant of Fragment 2A is present in molar excess relative to the coupling reagent in the coupling composition during the contacting of the protected variant of Fragment 2A and the coupling composition.

In some embodiments (e.g., certain embodiments in which a C-terminal carboxyl group of the protected variant of Fragment 2A is pre-activated), the method comprises:
contacting the protected variant of Fragment 2A with a coupling composition in solution to form a protected variant of Fragment 2A with an activated C-terminal carboxyl group (“activated Fragment 2A”); and
contacting the protected variant of Peptide B with the activated Fragment 2A in solution to form the protected variant of Peptide C, wherein the contacting does not occur in the presence of a coupling reagent.

In some embodiments, the protected variant of Fragment 2A is present in molar excess relative to the coupling reagent in the coupling composition during the contacting of the protected variant of Fragment 2A and the coupling composition.

In some embodiments, the contacting of the protected variant of Fragment 2A with the coupling composition and the contacting of the activated Fragment 2A with the protected variant of Peptide B occur in the same reaction vessel. In other embodiments, the contacting of the protected variant of Fragment 2A with the coupling composition and the contacting of the activated Fragment 2A with the protected variant of Peptide B occur in different reaction vessels.

In some embodiments, the activated Fragment 2A is contacted with the protected variant of Peptide B in the presence of a base and/or a coupling additive but not a coupling reagent (such as, e.g., in certain embodiments in which a C-terminal carboxyl group of the protected variant of Fragment 2A is pre-activated and the protected variant of Fragment 2A is present in molar excess relative to the coupling reagent in the coupling composition during the contacting to form activated Fragment 2A).

In some embodiments in which a C-terminal carboxyl group of the protected variant of Fragment 2A is pre-activated, the protected variant of Peptide B has a free N-terminal amino group and a free C-terminal carboxyl group.

In some embodiments, the coupling composition comprises a coupling reagent and a base. In some embodiments, the coupling composition comprises a coupling reagent (e.g., a carbodiimide, e.g., DIC, DCC, EDC) and a coupling additive (e.g., HOBt, 5-Cl—HOBt, Oxyma).

In some embodiments, the coupling composition comprises a coupling reagent, a coupling additive, and a base.

In some embodiments, the coupling reagent is a carbodiimide, such as, e.g., DIC, DCC, or EDC. In other embodiments, the coupling reagent is BOP—Cl.

In some embodiments (e.g., certain embodiments in which a C-terminal carboxyl group of the protected variant of Fragment 2A is activated in situ), the method comprises coupling the protected variant of Fragment 2A with the protected variant of Peptide B in the presence of a coupling composition. In some embodiments, the method comprises coupling the protected variant of Fragment 2A with the protected variant of Peptide B in solution in the presence of a coupling composition.

In some embodiments, the protected variant of Peptide B does not have a free carboxyl group at the C-terminus. In some embodiments, the C-terminus of the protected variant of Peptide B is protected. In some embodiments, the C-terminus of the protected variant of Peptide B is amidated. In some embodiments, the protected variant of Peptide B has a free amino group at the N-terminus. In preferred embodiments, the protected variant of Peptide B has a free amino group at the N-terminus and does not have a free carboxyl group at the C-terminus. In particularly preferred embodiments, the protected variant of Peptide B has a free amino group at the N-terminus and an amidated C-terminus.

In some embodiments, the protected variant of Fragment 2A does not have a free amino group at the N-terminus. In some embodiments, the N-terminus of the protected variant of Fragment 2A is protected (preferably Fmoc-protected). In some embodiments, the protected variant of Fragment 2A has a free carboxyl group at the C-terminus. In preferred embodiments, the protected variant of Fragment 2A has a free carboxyl group at the C-terminus and does not have a free amino group at the N-terminus. In particularly preferred embodiments, the protected variant of Fragment 2A has a free carboxyl group at the C-terminus and a protected (preferably Fmoc-protected) N-terminus.

In some embodiments, the protected variant of Peptide B has a free amino group at the N-terminus and does not have a free carboxyl group at the C-terminus, and the protected variant of Fragment 2A has a free carboxyl group at the C-terminus and a protected (preferably Fmoc-protected) N-terminus.

In some embodiments, the C-terminus of the protected variant of Peptide B is amidated, and the N-terminus of the protected variant of Fragment 2A is protected (preferably Fmoc-protected).

In preferred embodiments, the protected variant of Peptide B has a free amino group at the N-terminus and does not have a free carboxyl group at the C-terminus, and the protected variant of Fragment 2A has a free carboxyl group at the C-terminus and does not have a free amino group at the N-terminus. In particularly preferred embodiments, the protected variant of Peptide B has a free amino group at the N-terminus and an amidated C-terminus, and the protected variant of Fragment 2A has a free carboxyl group at the C-terminus and a protected (preferably Fmoc-protected) N-terminus.

In some embodiments, the protected variant of Peptide B has a free amino group at the N-terminus and an amidated C-terminus, and the protected variant of Fragment 2A has a free carboxyl group at the C-terminus and an Fmoc-protected N-terminus.

In some embodiments, the protected variant of Fragment 2A is Fmoc-Leu-Glu(OtBu)-Glu(OtBu)-Gln(Trt)-Ala-Ala-Lys(Boc)-Glu(OtBu)-Phe-Ile-OH (SEQ ID NO: 53).

In some embodiments, the protected variant of Peptide B is Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 140).

In some embodiments, the protected variant of Fragment 2A is Fmoc-Leu-Glu(OtBu)-Glu(OtBu)-Gln(Trt)-Ala-Ala-Lys(Boc)-Glu(OtBu)-Phe-Ile-OH (SEQ ID NO: 53), and the protected variant of Peptide B is Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 140).

In some embodiments, the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the coupling composition comprises DIC/Oxyma, EDC/Oxyma, PyBOP/collidine, PyOxim/collidine, TBTU/collidine, TBTU/DIEA, HCTU/DIEA, or PyOxim/DIEA. In some embodiments, the coupling composition comprises DIC/Oxyma, EDC/Oxyma, PyBOP/collidine, PyOxim/collidine, TBTU/collidine, TBTU/DIEA, HCTU/DIEA, or PyOxim/DIEA in DSMO.

In some embodiments, the coupling composition comprises DIC and Oxyma. In some embodiments, the coupling composition comprises DIC and Oxyma in DMSO.

In some embodiments, the protected variant of Fragment 2A is Fmoc-Leu-Glu(OtBu)-Glu(OtBu)-Gln(Trt)-Ala-Ala-Lys(Boc)-Glu(OtBu)-Phe-Ile-OH (SEQ ID NO: 53), the protected variant of Peptide B is Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 140), and the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the protected variant of Fragment 2A is Fmoc-Leu-Glu(OtBu)-Glu(OtBu)-Gln(Trt)-Ala-Ala-Lys(Boc)-Glu(OtBu)-Phe-Ile-OH (SEQ ID NO: 53), the protected variant of Peptide B is Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 140), and the coupling composition comprises DIC/Oxyma, EDC/Oxyma, PyBOP/collidine, PyOxim/collidine, TBTU/collidine, TBTU/DIEA, HCTU/DIEA, or PyOxim/DIEA (preferably DIC/Oxyma). In some embodiments, the coupling composition comprises DIC/Oxyma, EDC/Oxyma, PyBOP/collidine, PyOxim/collidine, TBTU/collidine, TBTU/DIEA, HCTU/DIEA, or PyOxim/DIEA (preferably DIC/Oxyma) in DMSO.

In some embodiments, the protected variant of Fragment 2A is Fmoc-Leu-Glu(OtBu)-Glu(OtBu)-Gln(Trt)-Ala-Ala-Lys(Boc)-Glu(OtBu)-Phe-Ile-OH (SEQ ID NO: 53), the protected variant of Peptide B is Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 140), and the coupling composition comprises DIC and Oxyma. In some embodiments, the coupling composition comprises DIC and Oxyma in DMSO.

In some embodiments, the coupling occurs at a temperature in the range of 0° C. to 60° C. In some embodiments, the coupling occurs at a temperature in the range of 20° C. to 50° C. In some embodiments, the coupling occurs at a temperature in the range of 20° C. to 30° C. In some embodiments, the coupling occurs at a temperature in the range of 30° C. to 50° C. In some embodiments, the coupling occurs at a temperature in the range of 35° C. to 45° C.

In some embodiments, the method comprises coupling the protected variant of Fragment 2A and the protected variant of Peptide B for a time in the range of 1 hour to 72 hours. In some embodiments, the time is in the range of 6 hours to 24 hours.

In some embodiments, the coupling occurs at a temperature in the range of 0° C. to 60° C. for a time in the range of 1 hour to 72 hours. In some embodiments, the coupling occurs at a temperature in the range of 0° C. to 60° C. for a time in the range of 6 hours to 24 hours. In some embodiments, the coupling occurs at a temperature in the range of 20° C. to 50° C. for a time in the range of 1 hour to 72 hours. In some embodiments, the coupling occurs at a temperature in the range of 20° C. to 50° C. for a time in the range of 6 hours to 24 hours.

In some embodiments, the protected variant of Fragment 2A is Fmoc-Leu-Glu(OtBu)-Glu(OtBu)-Gln(Trt)-Ala-Ala-Lys(Boc)-Glu(OtBu)-Phe-Ile-OH (SEQ ID NO: 53), the protected variant of Peptide B is Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 140), the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine, and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours).

In some embodiments, the protected variant of Fragment 2A is Fmoc-Leu-Glu(OtBu)-Glu(OtBu)-Gln(Trt)-Ala-Ala-Lys(Boc)-Glu(OtBu)-Phe-Ile-OH (SEQ ID NO: 53), the protected variant of Peptide B is Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 140), the coupling composition comprises DIC/Oxyma, EDC/Oxyma, PyBOP/collidine, PyOxim/collidine, TBTU/collidine, TBTU/DIEA, HCTU/DIEA, or PyOxim/DIEA (preferably DIC/Oxyma), and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours). In some embodiments, the coupling composition comprises DIC and Oxyma in DMSO.

In some embodiments, the method further comprises selectively deprotecting the N-terminus of the protected variant of Peptide C (e.g., using an Fmoc deprotection method described herein).

Also provided herein is a method of preparing Peptide C or a protected variant thereof, comprising coupling a protected variant of Fragment 2C with a protected variant of Peptide E to form a protected variant of Peptide C, wherein Fragment 2C comprises the amino acid sequence of SEQ ID NO: 17, Peptide E comprises the amino acid sequence of SEQ ID NO: 8, and Peptide C comprises the amino acid sequence of SEQ ID NO: 10.

In some embodiments, the protected variant of Fragment 2C is coupled with the protected variant of Peptide E in solution. In some embodiments, the protected variant of Fragment 2C is coupled with the protected variant of Peptide E under liquid phase peptide coupling conditions. In some embodiments, the protected variant of Fragment 2C is coupled with the protected variant of Peptide E under classical solution phase peptide coupling conditions. In some embodiments, the protected variant of Fragment 2C is coupled with the protected variant of Peptide E under tag-assisted liquid phase peptide coupling conditions.

In other embodiments, the protected variant of Fragment 2C is coupled with the protected variant of Peptide E on solid support. In some embodiments, the protected variant of Fragment 2C is coupled with the protected variant of Peptide E under solid phase peptide coupling conditions.

In some embodiments, a C-terminal carboxyl group of the protected variant of Fragment 2C is pre-activated. In other embodiments, a C-terminal carboxyl group of the protected variant of Fragment 2C is activated in situ.

In some embodiments, the method comprises:
contacting the protected variant of Fragment 2C with a coupling composition to form a protected variant of Fragment 2C with an activated C-terminal carboxyl group (“activated Fragment 2C”); and
contacting the protected variant of Peptide E with the activated Fragment 2C to form the protected variant of Peptide C.

In some embodiments, the method comprises:
contacting the protected variant of Fragment 2C with a coupling composition on solid support to form a protected variant of Fragment 2C with an activated C-terminal carboxyl group (“activated Fragment 2C”); and
contacting the protected variant of Peptide E with the activated Fragment 2C on solid support to form the protected variant of Peptide C.

In some embodiments, the method comprises:
contacting the protected variant of Fragment 2C with a coupling composition in solution to form a protected variant of Fragment 2C with an activated C-terminal carboxyl group (“activated Fragment 2C”); and
contacting the protected variant of Peptide E with the activated Fragment 2C in solution to form the protected variant of Peptide C.

In some embodiments (e.g., certain embodiments in which a C-terminal carboxyl group of the protected variant of Fragment 2C is pre-activated), the method comprises:
contacting the protected variant of Fragment 2C with a coupling composition to form a protected variant of Fragment 2C with an activated C-terminal carboxyl group (“activated Fragment 2C”); and
contacting the protected variant of Peptide E with the activated Fragment 2C to form the protected variant of Peptide C, wherein the contacting does not occur in the presence of a coupling reagent.

In some embodiments, the protected variant of Fragment 2C is present in molar excess relative to the coupling reagent in the coupling composition during the contacting of the protected variant of Fragment 2C and the coupling composition.

In some embodiments (e.g., certain embodiments in which a C-terminal carboxyl group of the protected variant of Fragment 2C is pre-activated), the method comprises:
contacting the protected variant of Fragment 2C with a coupling composition on solid support to form a protected variant of Fragment 2C with an activated C-terminal carboxyl group (“activated Fragment 2C”); and
contacting the protected variant of Peptide E with the activated Fragment 2C on solid support to form the protected variant of Peptide C, wherein the contacting does not occur in the presence of a coupling reagent.

In some embodiments, the protected variant of Fragment 2C is present in molar excess relative to the coupling reagent in the coupling composition during the contacting of the protected variant of Fragment 2C and the coupling composition.

In some embodiments (e.g., certain embodiments in which a C-terminal carboxyl group of the protected variant of Fragment 2C is pre-activated), the method comprises:
contacting the protected variant of Fragment 2C with a coupling composition in solution to form a protected variant of Fragment 2C with an activated C-terminal carboxyl group (“activated Fragment 2C”); and
contacting the protected variant of Peptide E with the activated Fragment 2C in solution to form the protected variant of Peptide C, wherein the contacting does not occur in the presence of a coupling reagent.

In some embodiments, the protected variant of Fragment 2C is present in molar excess relative to the coupling reagent in the coupling composition during the contacting of the protected variant of Fragment 2C and the coupling composition.

In some embodiments, the contacting of the protected variant of Fragment 2C with the coupling composition and the contacting of the activated Fragment 2C with the protected variant of Peptide E occur in the same reaction vessel. In other embodiments, the contacting of the protected variant of Fragment 2C with the coupling composition and the contacting of the activated Fragment 2C with the protected variant of Peptide E occur in different reaction vessels.

In some embodiments, the activated Fragment 2C is contacted with the protected variant of Peptide E in the presence of a base and/or a coupling additive but not a coupling reagent (such as, e.g., in certain embodiments in which a C-terminal carboxyl group of the protected variant of Fragment 2C is pre-activated and the protected variant of Fragment 2C is present in molar excess relative to the coupling reagent in the coupling composition during the contacting to form activated Fragment 2C).

In some embodiments in which a C-terminal carboxyl group of the protected variant of Fragment 2C is pre-activated, the protected variant of Peptide E has a free N-terminal amino group and a free C-terminal carboxyl group.

In some embodiments, the coupling composition comprises a coupling reagent and a base. In some embodiments, the coupling composition comprises a coupling reagent (e.g., a carbodiimide, e.g., DIC, DCC, EDC) and a coupling additive (e.g., HOBt, 5-Cl—HOBt, Oxyma).

In some embodiments, the coupling composition comprises a coupling reagent, a coupling additive, and a base.

In some embodiments, the coupling reagent is a carbodiimide, such as, e.g., DIC, DCC, or EDC. In other embodiments, the coupling reagent is BOP—Cl.

In some embodiments (e.g., certain embodiments in which a C-terminal carboxyl group of the protected variant of Fragment 2C is activated in situ), the method comprises coupling the protected variant of Fragment 2C with the protected variant of Peptide E in the presence of a coupling composition. In some embodiments, the method comprises coupling the protected variant of Fragment 2C with the protected variant of Peptide E in solution in the presence of a coupling composition.

In some embodiments, the protected variant of Peptide E does not have a free carboxyl group at the C-terminus. In some embodiments, the C-terminus of the protected variant of Peptide E is protected. In some embodiments, the C-terminus of the protected variant of Peptide E is amidated. In some embodiments, the protected variant of Peptide E has a free amino group at the N-terminus. In preferred embodiments, the protected variant of Peptide E has a free amino group at the N-terminus and does not have a free carboxyl group at the C-terminus. In particularly preferred embodiments, the protected variant of Peptide E has a free amino group at the N-terminus and an amidated C-terminus.

In some embodiments, the protected variant of Fragment 2C does not have a free amino group at the N-terminus. In some embodiments, the N-terminus of the protected variant of Fragment 2C is protected (preferably Fmoc-protected). In some embodiments, the protected variant of Fragment 2C has a free carboxyl group at the C-terminus. In preferred embodiments, the protected variant of Fragment 2C has a free carboxyl group at the C-terminus and does not have a free amino group at the N-terminus. In particularly preferred embodiments, the protected variant of Fragment 2C has a free carboxyl group at the C-terminus and a protected (preferably Fmoc-protected) N-terminus.

In some embodiments, the protected variant of Peptide E has a free amino group at the N-terminus and does not have a free carboxyl group at the C-terminus, and the protected variant of Fragment 2C has a free carboxyl group at the C-terminus and a protected (preferably Fmoc-protected) N-terminus.

In some embodiments, the C-terminus of the protected variant of Peptide E is amidated, and the N-terminus of the protected variant of Fragment 2C is protected (preferably Fmoc-protected).

In preferred embodiments, the protected variant of Peptide E has a free amino group at the N-terminus and does not have a free carboxyl group at the C-terminus, and the protected variant of Fragment 2C has a free carboxyl group at the C-terminus and does not have a free amino group at the N-terminus. In particularly preferred embodiments, the protected variant of Peptide E has a free amino group at the N-terminus and an amidated C-terminus, and the protected variant of Fragment 2C has a free carboxyl group at the C-terminus and a protected (preferably Fmoc-protected) N-terminus.

In some embodiments, the protected variant of Peptide E has a free amino group at the N-terminus and an amidated C-terminus, and the protected variant of Fragment 2C has a free carboxyl group at the C-terminus and an Fmoc-protected N-terminus.

In some embodiments, the protected variant of Fragment 2C is Fmoc-Leu-Glu(OtBu)-Glu(OtBu)-Gln(Trt)-Ala-Ala-Lys(Boc)-Glu(OtBu)-Phe-Ile-Ala-OH (SEQ ID NO: 55).

In some embodiments, the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 160).

In some embodiments, the protected variant of Fragment 2C is Fmoc-Leu-Glu(OtBu)-Glu(OtBu)-Gln(Trt)-Ala-Ala-Lys(Boc)-Glu(OtBu)-Phe-Ile-Ala-OH (SEQ ID NO: 55), and the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 160).

In some embodiments, the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the coupling composition comprises DIC/Oxyma, EDC/Oxyma, PyBOP/collidine, PyOxim/collidine, TBTU/collidine, TBTU/DIEA, HCTU/DIEA, or PyOxim/DIEA. In some embodiments, the coupling composition comprises DIC/Oxyma, EDC/Oxyma, PyBOP/collidine, PyOxim/collidine, TBTU/collidine, TBTU/DIEA, HCTU/DIEA, or PyOxim/DIEA in DSMO.

In some embodiments, the coupling composition comprises DIC and Oxyma. In some embodiments, the coupling composition comprises DIC and Oxyma in DMSO.

In some embodiments, the protected variant of Fragment 2C is Fmoc-Leu-Glu(OtBu)-Glu(OtBu)-Gln(Trt)-Ala-Ala-Lys(Boc)-Glu(OtBu)-Phe-Ile-Ala-OH (SEQ ID NO: 55), the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 160), and the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the protected variant of Fragment 2C is Fmoc-Leu-Glu(OtBu)-Glu(OtBu)-Gln(Trt)-Ala-Ala-Lys(Boc)-Glu(OtBu)-Phe-Ile-Ala-OH (SEQ ID NO: 55), the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 160), and the coupling composition comprises DIC/Oxyma, EDC/Oxyma, PyBOP/collidine, PyOxim/collidine, TBTU/collidine, TBTU/DIEA, HCTU/DIEA, or PyOxim/DIEA (preferably DIC/Oxyma). In some embodiments, the coupling composition comprises DIC/Oxyma, EDC/Oxyma, PyBOP/collidine, PyOxim/collidine, TBTU/collidine, TBTU/DIEA, HCTU/DIEA, or PyOxim/DIEA (preferably DIC/Oxyma) in DMSO.

In some embodiments, the protected variant of Fragment 2C is Fmoc-Leu-Glu(OtBu)-Glu(OtBu)-Gln(Trt)-Ala-Ala-Lys(Boc)-Glu(OtBu)-Phe-Ile-Ala-OH (SEQ ID NO: 55), the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 160), and the coupling composition comprises DIC and Oxyma. In some embodiments, the coupling composition comprises DIC and Oxyma in DMSO.

In some embodiments, the coupling occurs at a temperature in the range of 0° C. to 60° C. In some embodiments, the coupling occurs at a temperature in the range of 20° C. to 50° C. In some embodiments, the coupling occurs at a temperature in the range of 20° C. to 30° C. In some embodiments, the coupling occurs at a temperature in the range of 30° C. to 50° C. In some embodiments, the coupling occurs at a temperature in the range of 35° C. to 45° C.

In some embodiments, the method comprises coupling the protected variant of Fragment 2C and the protected variant of Peptide E for a time in the range of 1 hour to 72 hours. In some embodiments, the time is in the range of 6 hours to 24 hours.

In some embodiments, the coupling occurs at a temperature in the range of 0° C. to 60° C. for a time in the range of 1 hour to 72 hours. In some embodiments, the coupling occurs at a temperature in the range of 0° C. to 60° C. for a time in the range of 6 hours to 24 hours. In some embodiments, the coupling occurs at a temperature in the range of 20° C. to 50° C. for a time in the range of 1 hour to 72 hours. In some embodiments, the coupling occurs at a temperature in the range of 20° C. to 50° C. for a time in the range of 6 hours to 24 hours.

In some embodiments, the protected variant of Fragment 2C is Fmoc-Leu-Glu(OtBu)-Glu(OtBu)-Gln(Trt)-Ala-Ala-Lys(Boc)-Glu(OtBu)-Phe-Ile-Ala-OH (SEQ ID NO: 55), the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 160), the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine, and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours).

In some embodiments, the protected variant of Fragment 2C is Fmoc-Leu-Glu(OtBu)-Glu(OtBu)-Gln(Trt)-Ala-Ala-Lys(Boc)-Glu(OtBu)-Phe-Ile-Ala-OH (SEQ ID NO: 55), the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 160), the coupling composition comprises DIC/Oxyma, EDC/Oxyma, PyBOP/collidine, PyOxim/collidine, TBTU/collidine, TBTU/DIEA, HCTU/DIEA, or PyOxim/DIEA (preferably DIC/Oxyma), and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours). In some embodiments, the coupling composition comprises DIC and Oxyma in DMSO.

In some embodiments, the method further comprises selectively deprotecting the N-terminus of the protected variant of Peptide C (e.g., using an Fmoc deprotection method described herein).

Also provided herein is a method of preparing Peptide A or a protected variant thereof, comprising coupling a protected variant of Fragment 1A with a protected variant of Peptide C to form a protected variant of Peptide A, wherein Fragment 1A comprises the amino acid sequence of SEQ ID NO: 11, Peptide C comprises the amino acid sequence of SEQ ID NO: 10, and Peptide A comprises the amino acid sequence of SEQ ID NO: 7. In some embodiments, the method further comprises deprotecting the protected variant of Peptide A to isolate Peptide A. In some embodiments, the method further comprises adding a bromoacetyl moiety to the Lys at the C-terminus of Peptide A or a protected variant thereof.

In some embodiments, the protected variant of Fragment 1A is coupled with the protected variant of Peptide C in solution. In some embodiments, the protected variant of Fragment 1A is coupled with the protected variant of Peptide C under liquid phase peptide coupling conditions. In some embodiments, the protected variant of Fragment 1A is coupled with the protected variant of Peptide C under classical solution phase peptide coupling conditions. In some embodiments, the protected variant of Fragment 1A is coupled with the protected variant of Peptide C under tag-assisted liquid phase peptide coupling conditions.

In other embodiments, the protected variant of Fragment 1A is coupled with the protected variant of Peptide Con solid support. In some embodiments, the protected variant of Fragment 1A is coupled with the protected variant of Peptide C under solid phase peptide coupling conditions.

In some embodiments, a C-terminal carboxyl group of the protected variant of Fragment 1A is pre-activated. In other embodiments, a C-terminal carboxyl group of the protected variant of Fragment 1A is activated in situ.

In some embodiments, the method comprises:
contacting the protected variant of Fragment 1A with a coupling composition to form a protected variant of Fragment 1A with an activated C-terminal carboxyl group (“activated Fragment 1A”); and
contacting the protected variant of Peptide C with the activated Fragment 1A to form the protected variant of Peptide A.

In some embodiments, the method comprises:
contacting the protected variant of Fragment 1A with a coupling composition on solid support to form a protected variant of Fragment 1A with an activated C-terminal carboxyl group (“activated Fragment 1A”); and
contacting the protected variant of Peptide C with the activated Fragment 1A on solid support to form the protected variant of Peptide A.

In some embodiments, the method comprises:
contacting the protected variant of Fragment 1A with a coupling composition in solution to form a protected variant of Fragment 1A with an activated C-terminal carboxyl group (“activated Fragment 1A”); and
contacting the protected variant of Peptide C with the activated Fragment 1A in solution to form the protected variant of Peptide A.

In some embodiments (e.g., certain embodiments in which a C-terminal carboxyl group of the protected variant of Fragment 1A is pre-activated), the method comprises:
contacting the protected variant of Fragment 1A with a coupling composition to form a protected variant of Fragment 1A with an activated C-terminal carboxyl group (“activated Fragment 1A”); and
contacting the protected variant of Peptide C with the activated Fragment 1A to form the protected variant of Peptide A, wherein the contacting does not occur in the presence of a coupling reagent.

In some embodiments, the protected variant of Fragment 1A is present in molar excess relative to the coupling reagent in the coupling composition during the contacting of the protected variant of Fragment 1A and the coupling composition.

In some embodiments (e.g., certain embodiments in which a C-terminal carboxyl group of the protected variant of Fragment 1A is pre-activated), the method comprises:
contacting the protected variant of Fragment 1A with a coupling composition on solid support to form a protected variant of Fragment 1A with an activated C-terminal carboxyl group (“activated Fragment 1A”); and
contacting the protected variant of Peptide C with the activated Fragment 1A on solid support to form the protected variant of Peptide A, wherein the contacting does not occur in the presence of a coupling reagent.

In some embodiments, the protected variant of Fragment 1A is present in molar excess relative to the coupling reagent in the coupling composition during the contacting of the protected variant of Fragment 1A and the coupling composition.

In some embodiments (e.g., certain embodiments in which a C-terminal carboxyl group of the protected variant of Fragment 1A is pre-activated), the method comprises:
contacting the protected variant of Fragment 1A with a coupling composition in solution to form a protected variant of Fragment 1A with an activated C-terminal carboxyl group (“activated Fragment 1A”); and
contacting the protected variant of Peptide C with the activated Fragment 1A in solution to form the protected variant of Peptide A, wherein the contacting does not occur in the presence of a coupling reagent.

In some embodiments, the protected variant of Fragment 1A is present in molar excess relative to the coupling reagent in the coupling composition during the contacting of the protected variant of Fragment 1A and the coupling composition.

In some embodiments, the contacting of the protected variant of Fragment 1A with the coupling composition and the contacting of the activated Fragment 1A with the protected variant of Peptide C occur in the same reaction vessel. In other embodiments, the contacting of the protected variant of Fragment 1A with the coupling composition and the contacting of the activated Fragment 1A with the protected variant of Peptide C occur in different reaction vessels.

In some embodiments, the activated Fragment 1A is contacted with the protected variant of Peptide C in the presence of a base and/or a coupling additive but not a coupling reagent (such as, e.g., in certain embodiments in which a C-terminal carboxyl group of the protected variant of Fragment 1A is pre-activated and the protected variant of Fragment 1A is present in molar excess relative to the coupling reagent in the coupling composition during the contacting to form activated Fragment 1A).

In some embodiments in which a C-terminal carboxyl group of the protected variant of Fragment 1A is pre-activated, the protected variant of Peptide C has a free N-terminal amino group and a free C-terminal carboxyl group.

In some embodiments, the coupling composition comprises a coupling reagent and a base. In some embodiments, the coupling composition comprises a coupling reagent (e.g., a carbodiimide, e.g., DIC, DCC, EDC) and a coupling additive (e.g., HOBt, 5-Cl—HOBt, Oxyma).

In some embodiments, the coupling composition comprises a coupling reagent, a coupling additive, and a base.

In some embodiments, the coupling reagent is a carbodiimide, such as, e.g., DIC, DCC, or EDC. In other embodiments, the coupling reagent is BOP—Cl.

In some embodiments (e.g., certain embodiments in which a C-terminal carboxyl group of the protected variant of Fragment 1A is activated in situ), the method comprises coupling the protected variant of Fragment 1A with the protected variant of Peptide C in the presence of a coupling composition. In some embodiments, the method comprises coupling the protected variant of Fragment 1A with the protected variant of Peptide C in solution in the presence of a coupling composition.

In some embodiments, the protected variant of Peptide C does not have a free carboxyl group at the C-terminus. In some embodiments, the C-terminus of the protected variant of Peptide C is protected. In some embodiments, the C-terminus of the protected variant of Peptide C is amidated. In some embodiments, the protected variant of Peptide C has a free amino group at the N-terminus. In preferred embodiments, the protected variant of Peptide C has a free amino group at the N-terminus and does not have a free carboxyl group at the C-terminus. In particularly preferred embodiments, the protected variant of Peptide C has a free amino group at the N-terminus and an amidated C-terminus.

In some embodiments, the protected variant of Fragment 1A does not have a free amino group at the N-terminus. In some embodiments, the N-terminus of the protected variant of Fragment 1A is protected (preferably Boc-protected). In some embodiments, the protected variant of Fragment 1A has a free carboxyl group at the C-terminus. In preferred embodiments, the protected variant of Fragment 1A has a free carboxyl group at the C-terminus and does not have a free amino group at the N-terminus. In particularly preferred embodiments, the protected variant of Fragment 1A has a free carboxyl group at the C-terminus and a protected (preferably Boc-protected) N-terminus.

In some embodiments, the protected variant of Peptide C has a free amino group at the N-terminus and does not have a free carboxyl group at the C-terminus, and the protected variant of Fragment 1A has a free carboxyl group at the C-terminus and a protected (preferably Boc-protected) N-terminus.

In some embodiments, the C-terminus of the protected variant of Peptide C is amidated, and the N-terminus of the protected variant of Fragment 1A is protected (preferably Boc-protected).

In preferred embodiments, the protected variant of Peptide C has a free amino group at the N-terminus and does not have a free carboxyl group at the C-terminus, and the protected variant of Fragment 1A has a free carboxyl group at the C-terminus and does not have a free amino group at the N-terminus. In particularly preferred embodiments, the protected variant of Peptide C has a free amino group at the N-terminus and an amidated C-terminus, and the protected variant of Fragment 1A has a free carboxyl group at the C-terminus and a protected (preferably Boc-protected) N-terminus.

In some embodiments, the protected variant of Peptide C has a free amino group at the N-terminus and an amidated C-terminus, and the protected variant of Fragment 1A has a free carboxyl group at the C-terminus and a Boc-protected N-terminus.

In some embodiments, the protected variant of Fragment 1A is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH (SEQ ID NO: 95).

In some embodiments, the protected variant of Peptide C is Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 96).

In some embodiments, the protected variant of Fragment 1A is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH (SEQ ID NO: 95), and the protected variant of Peptide C is Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 96).

In some embodiments, the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the coupling composition comprises DIC/Oxyma, EDC/Oxyma, PyBOP/collidine, PyOxim/collidine, or TBTU/collidine. In some embodiments, the coupling composition.

In some embodiments, the coupling composition comprises DIC and Oxyma. In some embodiments, the coupling composition comprises DIC and Oxyma in DMSO. In some embodiments, the coupling composition comprises DIC and Oxyma in DMF.

In some embodiments, the protected variant of Fragment 1A is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH (SEQ ID NO: 95), the protected variant of Peptide C is Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 96), and the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the protected variant of Fragment 1A is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH (SEQ ID NO: 95), the protected variant of Peptide C is Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 96), and the coupling composition comprises DIC/Oxyma, EDC/Oxyma, PyBOP/collidine, PyOxim/collidine, or TBTU/collidine (preferably DIC/Oxyma). In some embodiments, the coupling composition comprises DIC/Oxyma, EDC/Oxyma, PyBOP/collidine, PyOxim/collidine, or TBTU/collidine (preferably DIC/Oxyma) in DMSO. In some embodiments, the coupling composition comprises DIC and Oxyma in DMSO or DMF. In some embodiments, the coupling composition comprises DIC and Oxyma in DMSO. In some embodiments, the coupling composition comprises DIC and Oxyma in DMF.

In some embodiments, the protected variant of Fragment 1A is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH (SEQ ID NO: 95), and the coupling composition comprises DIC and Oxyma. In some embodiments, the coupling composition comprises DIC and Oxyma in DMSO or DMF. In some embodiments, the coupling composition comprises DIC and Oxyma in DMSO. In some embodiments, the coupling composition comprises DIC and Oxyma in DMF.

In some embodiments, the coupling occurs at a temperature in the range of 0° C. to 60° C. In some embodiments, the coupling occurs at a temperature in the range of 20° C. to 50° C. In some embodiments, the coupling occurs at a temperature in the range of 20° C. to 30° C. In some embodiments, the coupling occurs at a temperature in the range of 30° C. to 50° C. In some embodiments, the coupling occurs at a temperature in the range of 35° C. to 45° C.

In some embodiments, the method comprises coupling the protected variant of Fragment 1A and the protected variant of Peptide C for a time in the range of 1 hour to 72 hours. In some embodiments, the time is in the range of 6 hours to 24 hours.

In some embodiments, the coupling occurs at a temperature in the range of 0° C. to 60° C. for a time in the range of 1 hour to 72 hours. In some embodiments, the coupling occurs at a temperature in the range of 0° C. to 60° C. for a time in the range of 6 hours to 24 hours. In some embodiments, the coupling occurs at a temperature in the range of 20° C. to 50° C. for a time in the range of 1 hour to 72 hours. In some embodiments, the coupling occurs at a temperature in the range of 20° C. to 50° C. for a time in the range of 6 hours to 24 hours.

In some embodiments, the protected variant of Fragment 1A is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH (SEQ ID NO: 95), the protected variant of Peptide C is Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 96), the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine, and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours).

In some embodiments, the protected variant of Fragment 1A is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH (SEQ ID NO: 95), the protected variant of Peptide C is Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 96), the coupling composition comprises DIC/Oxyma, EDC/Oxyma, PyBOP/collidine, PyOxim/collidine, or TBTU/collidine (preferably DIC/Oxyma), and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours). In some embodiments, the coupling composition comprises DIC and Oxyma in DMSO or DMF. In some embodiments, the coupling composition comprises DIC and Oxyma in DMSO. In some embodiments, the coupling composition comprises DIC and Oxyma in DMF.

Also provided herein is a method of preparing Peptide A or a protected variant thereof, comprising:
coupling or having coupled a protected variant of Fragment 2A with a protected variant of Peptide B to form a first protected variant of Peptide C, wherein the protected variant of Fragment 2A has a free carboxyl group at the C-terminus and a protected (preferably Fmoc-protected) N-terminus, the protected variant of Peptide B has a free amino group at the N-terminus, Fragment 2A comprises the amino acid sequence of SEQ ID NO: 12, Peptide B comprises the amino acid sequence of SEQ ID NO: 9, and Peptide C comprises the amino acid sequence of SEQ ID NO: 10;
selectively deprotecting or having selectively deprotected the N-terminus of the first protected variant of Peptide C to form a second protected variant of Peptide C with a free amino group at the N-terminus; and
coupling or having coupled a protected variant of Fragment 1A with the second protected variant of Peptide C to form a protected variant of Peptide A, wherein the protected variant of Fragment 1A has a free carboxyl group at the C-terminus and a protected (preferably Boc-protected) N-terminus, Fragment 1A comprises the amino acid sequence of SEQ ID NO: 11, and Peptide A comprises the amino acid sequence of SEQ ID NO: 7.

In some embodiments, the method further comprises deprotecting or having deprotected the protected variant of Peptide A to isolate Peptide A.

In some embodiments, the method further comprises adding or having added a bromoacetyl moiety to the Lys at the C-terminus of Peptide A or a protected variant thereof.

In some embodiments, the protected variant of Fragment 2A and the protected variant of Peptide B are coupled under conditions described herein.

In some embodiments, the protected variant of Fragment 1A and the second protected variant of Peptide C are coupled under conditions described herein.

In some embodiments, the protected variant of Fragment 2A and the protected variant of Peptide B are coupled under conditions described above, and the protected variant of Fragment 1A and the second protected variant of Peptide C are coupled under conditions described herein.

In some embodiments, the N-terminus of the first protected variant of Peptide C is Fmoc-protected, and the selective deprotection comprises contacting the first protected variant of Peptide C with an amine base. In some embodiments, the amine base is selected from piperidine, piperazine, and morpholine (e.g., 5-30% (v/V) piperidine, piperazine, or morpholine). In some embodiments, the amine base is piperidine. In some embodiments, the N-terminus of the first protected variant of Peptide C is Fmoc-protected, and the selective deprotection comprises contacting the first protected variant of Peptide C with piperidine in DMF, optionally in the presence of DBU.

In some embodiments, the N-terminus of the first protected variant of Peptide C is Boc-protected, and the selective deprotection comprises contacting the first protected variant of Peptide C with an acid (preferably an acid selected from TFA, HCl, and HBr). In some embodiments, the first protected variant of Peptide C is contacted with TFA (e.g., neat TFA or TFA in DCM, e.g., 20-50% (v/v) TFA in DCM). In some embodiments, the first protected variant of Peptide C is contacted with HCl (e.g., HCl in DCM, DCE, toluene, or dioxane, e.g., 3-4 M HCl in DCM or dioxane).

In some embodiments, the N-terminus of the first protected variant of Peptide C is Boc-protected, and the selective deprotection comprises contacting the first protected variant of Peptide C with an acid (preferably an acid selected from TFA, HCl, and HBr) in the presence of a scavenger. In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some preferred embodiments, the scavenger is a hydrosilane. In some particularly preferred embodiments, the scavenger is TIS. In some embodiments, the first protected variant of Peptide C is contacted with TFA (e.g., neat TFA or TFA in DCM, e.g., 20-50% (v/v) TFA in DCM) in the presence of a scavenger (preferably TIS). In some embodiments, the first protected variant of Peptide C is contacted with HCl (e.g., HCl in DCM, DCE, toluene, or dioxane, e.g., 3-4 M HCl in DCM or dioxane) in the presence of a scavenger.

In some embodiments, the N-terminus of the first protected variant of Peptide C is Trt-protected, and the selective deprotection comprises contacting the first protected variant of Peptide C with a Brønsted acid or a Lewis acid (preferably TFA, HCl, or dichloroacetic acid, most preferably TFA), optionally in the presence of a scavenger (preferably TIS), to selectively remove the trityl protecting group at the N-terminus. In some embodiments, the first protected variant of Peptide C is contacted with a Brønsted acid (preferably a dilute Brønsted acid). In some embodiments, the Brønsted acid is selected from TFA, HCl, and dichloroacetic acid (preferably dilute TFA, HCl, or dichloroacetic acid). In other embodiments, the first protected variant of Peptide C is contacted with a Lewis acid (preferably a dilute Lewis acid).

In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some embodiments, the scavenger is a hydrosilane. In some embodiments, the scavenger is TIS.

In some embodiments, the N-terminus of the first protected variant of Peptide C is Trt-protected, and the selective deprotection comprises contacting the first protected variant of Peptide C with HCl. In some embodiments, the selective deprotection comprises contacting the first protected variant of Peptide C with dilute HCl.

In some embodiments, the N-terminus of the first protected variant of Peptide C is Trt-protected, and the selective deprotection comprises contacting the first protected variant of Peptide C with TFA in the presence of TIS. In some embodiments, the selective deprotection comprises contacting the first protected variant of Peptide C with dilute TFA in the presence of TIS.

Also provided herein is a method of preparing Peptide A or a protected variant thereof, comprising:
(A)
(i) (a) coupling a protected variant of Fragment 3 with a protected variant of Fragment 4 to form a first protected variant of Peptide E, wherein the protected variant of Fragment 3 has a free carboxyl group at the C-terminus and a protected (preferably Fmoc-protected) N-terminus, the protected variant of Fragment 4 has a free amino group at the N-terminus, Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8; (b) selectively deprotecting the N-terminus of the first protected variant of Peptide E from step (A) (i) (a) to form a second protected variant of Peptide E with a free amino group at the N-terminus; (c) coupling the second protected variant of Peptide E from step (A) (i) (b) with an N-protected alanine to form a first protected variant of Peptide B, wherein Peptide B comprises the amino acid sequence of SEQ ID NO: 9; and (d) selectively deprotecting the N-terminus of the first protected variant of Peptide B from step (A) (i) (c) to form a second protected variant of Peptide B with a free amino group at the N-terminus; OR
(ii) (a) coupling a protected variant of Fragment 3A with a protected variant of Fragment 4 to form a first protected variant of Peptide B, wherein the protected variant of Fragment 3A has a free carboxyl group at the C-terminus and a protected (preferably Fmoc-protected) N-terminus, the protected variant of Fragment 4 has a free amino group at the N-terminus, Fragment 3A comprises the amino acid sequence of SEQ ID NO: 13, Fragment 4 comprises the amino acid sequence of SEQ ID NO: 4, and Peptide B comprises the amino acid sequence of SEQ ID NO: 9; and (b) selectively deprotecting the N-terminus of the first protected variant of Peptide B from step (A) (ii) (a) to form a second protected variant of Peptide B with a free amino group at the N-terminus; OR (iii) (a) coupling a protected variant of Fragment 5 with a protected variant of Fragment 6 to form a first protected variant of Peptide B, wherein the protected variant of Fragment 5 has a free carboxyl group at the C-terminus and a protected (preferably Trt- or Fmoc-protected, more preferably Trt-protected) N-terminus, the protected variant of Fragment 6 has a free amino group at the N-terminus, Fragment 5 comprises the amino acid sequence of SEQ ID NO: 5, Fragment 6 comprises the amino acid sequence of SEQ ID NO: 6, and Peptide B comprises the amino acid sequence of SEQ ID NO: 9; and (b) selectively deprotecting the N-terminus of the first protected variant of Peptide B from step (A) (iii) (a) to form a second protected variant of Peptide B with a free amino group at the N-terminus;
(B) (i) coupling a protected variant of Fragment 2A with the second protected variant of Peptide B from step (A) to form a first protected variant of Peptide C, wherein the protected variant of Fragment 2A has a free carboxyl group at the C-terminus and a protected (preferably Fmoc-protected) N-terminus, Fragment 2A comprises the amino acid sequence of SEQ ID NO: 12, and Peptide C comprises the amino acid sequence of SEQ ID NO: 10; and (ii) selectively deprotecting the N-terminus of the first protected variant of Peptide C from step (B) (i) to form a second protected variant of Peptide C with a free amino group at the N-terminus; and
(C) coupling a protected variant of Fragment 1A with the second protected variant of Peptide C from step (B) (ii) to form a protected variant of Peptide A, wherein the protected variant of Fragment 1A has a free carboxyl group at the C-terminus and a protected (preferably Boc-protected) N-terminus, Fragment 1A comprises the amino acid sequence of SEQ ID NO: 11, and Peptide A comprises the amino acid sequence of SEQ ID NO: 7.

In some embodiments, the method further comprises deprotecting the protected variant of Peptide A to isolate Peptide A.

In some embodiments, the method further comprises adding a bromoacetyl moiety to the Lys at the C-terminus of Peptide A or a protected variant thereof.

In some embodiments, the method comprises step (A) (i), step (B), and step (C). In other embodiments, the method comprises step (A) (ii), step (B), and step (C). In other preferred embodiments, the method comprises step (A) (iii), step (B), and step (C).

In some embodiments, the method comprises step (A) (i), step (B), and step (C), wherein each coupling is performed under liquid phase peptide coupling conditions. In other embodiments, the method comprises step (A) (ii), step (B), and step (C), wherein each coupling is performed under liquid phase peptide coupling conditions. In other preferred embodiments, the method comprises step (A) (iii), step (B), and step (C), wherein each coupling is performed under liquid phase peptide coupling conditions.

In some embodiments, the method comprises step (A) (i), step (B), and step (C), wherein each coupling is performed under solid phase peptide coupling conditions. In other embodiments, the method comprises step (A) (ii), step (B), and step (C), wherein each coupling is performed under solid phase peptide coupling conditions. In other embodiments, the method comprises step (A) (iii), step (B), and step (C), wherein each coupling is performed under solid phase peptide coupling conditions.

In some embodiments, the protected variant of Fragment 3 and the protected variant of Fragment 4 are coupled in step (A) (i) (a) under conditions described herein.

In some embodiments, the second protected variant of Peptide E and the N-protected alanine are coupled in step (A) (i) (c) under conditions described herein.

In some embodiments, the protected variant of Fragment 3A and the protected variant of Fragment 4 are coupled in step (A) (ii) (a) are coupled under conditions described herein.

In some embodiments, the protected variant of Fragment 5 and the protected variant of Fragment 6 are coupled in step (A) (iii) (a) are coupled under conditions described herein.

In some embodiments, the protected variant of Fragment 2A and the second protected variant of Peptide B are coupled in step (B) are coupled under conditions described herein.

In some embodiments, the protected variant of Peptide 1A and the second protected variant of Peptide C are coupled in step (C) are coupled under conditions described herein.

In some embodiments, the method comprises step (A) (i), the N-terminus of the first protected variant of Peptide E is Fmoc-protected, and the selective deprotection of step (A) (i) (b) comprises contacting the first protected variant of Peptide E with an amine base to selectively remove the Fmoc protecting group at the N-terminus. In some embodiments, the amine base is selected from piperidine, piperazine, and morpholine (e.g., 5-30% (v/v) piperidine, piperazine, or morpholine). In some embodiments, the amine base is piperidine. In some embodiments, the method comprises step (A) (i), the N-terminus of the first protected variant of Peptide E is Fmoc-protected, and the selective deprotection of step (A) (i) (b) comprises contacting the first protected variant of Peptide E with piperidine in DMF, optionally in the presence of DBU.

In some embodiments, the method comprises step (A) (i), the N-terminus of the first protected variant of Peptide E is Boc-protected, and the selective deprotection of step (A) (i) (b) comprises contacting the first protected variant of Peptide E with an acid (preferably an acid selected from TFA, HCl, and HBr) to selectively remove the Boc protecting group at the N-terminus. In some embodiments, the first protected variant of Peptide E is contacted with TFA (e.g., neat TFA or TFA in DCM, e.g., 20-50% (v/v) TFA in DCM). In some embodiments, the first protected variant of Peptide E is contacted with HCl (e.g., HCl in DCM, DCE, toluene, or dioxane, e.g., 3-4 M HCl in DCM or dioxane).

In some embodiments, the method comprises step (A) (i), the N-terminus of the first protected variant of Peptide E is Boc-protected, and the selective deprotection of step (A) (i) (b) comprises contacting the first protected variant of Peptide E with an acid (preferably an acid selected from TFA, HCl, and HBr) in the presence of a scavenger. In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some preferred embodiments, the scavenger is a hydrosilane. In some particularly preferred embodiments, the scavenger is TIS. In some embodiments, the first protected variant of Peptide E is contacted with TFA (e.g., neat TFA or TFA in DCM, e.g., 20-50% (v/v) TFA in DCM) in the presence of a scavenger (preferably TIS). In some embodiments, the first protected variant of Peptide E is contacted with HCl (e.g., HCl in DCM, DCE, toluene, or dioxane, e.g., 3-4 M HCl in DCM or dioxane) in the presence of a scavenger.

In some embodiments, the method comprises step (A) (i), the N-terminus of the first protected variant of Peptide E is Trt-protected, and the selective deprotection of step (A) (i) (b) comprises contacting the first protected variant of Peptide E with a Brønsted acid or a Lewis acid (preferably TFA, HCl, or dichloroacetic acid, most preferably TFA), optionally in the presence of a scavenger (preferably TIS), to selectively remove the trityl protecting group at the N-terminus. In some embodiments, the first protected variant of Peptide E is contacted with a Brønsted acid (preferably a dilute Brønsted acid). In some embodiments, the Brønsted acid is selected from TFA, HCl, and dichloroacetic acid (preferably dilute TFA, HCl, or dichloroacetic acid). In other embodiments, the first protected variant of Peptide E is contacted with a Lewis acid (preferably a dilute Lewis acid).

In some embodiments, the method comprises step (A) (i), the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some embodiments, the scavenger is a hydrosilane. In some embodiments, the scavenger is TIS.

In some embodiments, the method comprises step (A) (i), the N-terminus of the first protected variant of Peptide E is Trt-protected, and the selective deprotection of step (A) (i) (b) comprises contacting the first protected variant of Peptide E with HCl. In some embodiments, the selective deprotection of step (A) (i) (b) comprises contacting the first protected variant of Peptide E with dilute HCl.

In some embodiments, the method comprises step (A) (i), the N-terminus of the first protected variant of Peptide E is Trt-protected, and the selective deprotection of step (A) (i) (b) comprises contacting the first protected variant of Peptide E with TFA in the presence of TIS. In some embodiments, the selective deprotection of step (A) (i) (b) comprises contacting the first protected variant of Peptide E with dilute TFA in the presence of TIS.

In some embodiments, the N-terminus of the first protected variant of Peptide B is Fmoc-protected, and the selective deprotection of step (A) (i) (d), step (A) (ii) (b), or step (A) (iii) (b) comprises contacting the first protected variant of Peptide B with an amine base to selectively remove the Fmoc protecting group at the N-terminus. In some embodiments, the amine base is selected from piperidine, piperazine, and morpholine (e.g., 5-30% (v/v) piperidine, piperazine, or morpholine). In some embodiments, the amine base is piperidine. In some embodiments, the N-terminus of the first protected variant of Peptide B is Fmoc-protected, and the selective deprotection of step (A) (i) (d), step (A) (ii) (b), or step (A) (iii) (b) comprises contacting the first protected variant of Peptide B with piperidine in DMF, optionally in the presence of DBU.

In some embodiments, the N-terminus of the first protected variant of Peptide B is Boc-protected, and the selective deprotection of step (A) (i) (d), step (A) (ii) (b), or step (A) (iii) (b) comprises contacting the first protected variant of Peptide B with an acid (preferably an acid selected from TFA, HCl, and HBr) to selectively remove the Boc protecting group at the N-terminus. In some embodiments, the first protected variant of Peptide B is contacted with TFA (e.g., neat TFA or TFA in DCM, e.g., 20-50% (v/v) TFA in DCM). In some embodiments, the first protected variant of Peptide B is contacted with HCl (e.g., HCl in DCM, DCE, toluene, or dioxane, e.g., 3-4 M HCl in DCM or dioxane).

In some embodiments, the N-terminus of the first protected variant of Peptide B is Boc-protected, and the selective deprotection of step (A) (i) (d), step (A) (ii) (b), or step (A) (iii) (b) comprises contacting the first protected variant of Peptide B with an acid (preferably an acid selected from TFA, HCl, and HBr) in the presence of a scavenger. In some embodiments, the scavenger is selected from hydrosilanes (e.g., TIS, triethylsilane (TES), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some preferred embodiments, the scavenger is a hydrosilane. In some particularly preferred embodiments, the scavenger is TIS. In some embodiments, the first protected variant of Peptide B is contacted with TFA (e.g., neat TFA or TFA in DCM, e.g., 20-50% (v/v) TFA in DCM) in the presence of a scavenger (preferably TIS). In some embodiments, the first protected variant of Peptide B is contacted with HCl (e.g., HCl in DCM, DCE, toluene, or dioxane, e.g., 3-4 M HCl in DCM or dioxane) in the presence of a scavenger.

In some embodiments, the N-terminus of the first protected variant of Peptide B is Trt-protected, and the selective deprotection of step (A) (i) (d), step (A) (ii) (b), or step (A) (iii) (b) comprises contacting the first protected variant of Peptide B with a Brønsted acid or a Lewis acid (preferably TFA, HCl, or dichloroacetic acid, most preferably TFA), optionally in the presence of a scavenger (preferably TIS), to selectively remove the trityl protecting group at the N-terminus. In some embodiments, the first protected variant of Peptide B is contacted with a Brønsted acid (preferably a dilute Brønsted acid). In some embodiments, the Brønsted acid is selected from TFA, HCl, and dichloroacetic acid (preferably dilute TFA, HCl, or dichloroacetic acid). In other embodiments, the first protected variant of Peptide B is contacted with a Lewis acid (preferably a dilute Lewis acid).

In some embodiments, the scavenger is selected from hydrosilanes (e.g., TIS, triethylsilane (TES), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some embodiments, the scavenger is a hydrosilane. In some embodiments, the scavenger is TIS.

In some embodiments, the N-terminus of the first protected variant of Peptide B is Trt-protected, and the selective deprotection of step (A) (i) (d), step (A) (ii) (b), or step (A) (iii) (b) comprises contacting the first protected variant of Peptide B with HCl. In some embodiments, the selective deprotection of step (A) (i) (d), step (A) (ii) (b), or step (A) (iii) (b) comprises contacting the first protected variant of Peptide B with dilute HCl.

In some embodiments, the N-terminus of the first protected variant of Peptide B is Trt-protected, and the selective deprotection of step (A) (i) (d), step (A) (ii) (b), or step (A) (iii) (b) comprises contacting the first protected variant of Peptide B with TFA in the presence of TIS. In some embodiments, the selective deprotection of step (A) (i) (d), step (A) (ii) (b), or step (A) (iii) (b) comprises contacting the first protected variant of Peptide B with dilute TFA in the presence of TIS.

Also provided herein is a method of preparing Peptide F or a protected variant thereof, comprising coupling a protected variant of Fragment 2 with a protected variant of Peptide E to form a protected variant of Peptide F, wherein Fragment 2 comprises the amino acid sequence of SEQ ID NO: 2, Peptide E comprises the amino acid sequence of SEQ ID NO: 8, and Peptide F comprises the amino acid sequence of SEQ ID NO: 14.

In some embodiments, the protected variant of Fragment 2 is coupled with the protected variant of Peptide E in solution. In some embodiments, the protected variant of Fragment 2 is coupled with the protected variant of Peptide E under liquid phase peptide coupling conditions. In some embodiments, the protected variant of Fragment 2 is coupled with the protected variant of Peptide E under classical solution phase peptide coupling conditions. In some embodiments, the protected variant of Fragment 2 is coupled with the protected variant of Peptide E under tag-assisted liquid phase peptide coupling conditions.

In other embodiments, the protected variant of Fragment 2 is coupled with the protected variant of Peptide E on solid support. In some embodiments, the protected variant of Fragment 2 is coupled with the protected variant of Peptide E under solid phase peptide coupling conditions.

In some embodiments, a C-terminal carboxyl group of the protected variant of Fragment 2 is pre-activated. In other embodiments, a C-terminal carboxyl group of the protected variant of Fragment 2 is activated in situ.

In some embodiments, the method comprises:
contacting the protected variant of Fragment 2 with a coupling composition to form a protected variant of Fragment 2 with an activated C-terminal carboxyl group (“activated Fragment 2”); and
contacting the protected variant of Peptide E with the activated Fragment 2 to form the protected variant of Peptide F.

In some embodiments, the method comprises:
contacting the protected variant of Fragment 2 with a coupling composition on solid support to form a protected variant of Fragment 2 with an activated C-terminal carboxyl group (“activated Fragment 2”); and
contacting the protected variant of Peptide E with the activated Fragment 2 on solid support to form the protected variant of Peptide F.

In some embodiments, the method comprises:
contacting the protected variant of Fragment 2 with a coupling composition in solution to form a protected variant of Fragment 2 with an activated C-terminal carboxyl group (“activated Fragment 2”); and
contacting the protected variant of Peptide E with the activated Fragment 2 in solution to form the protected variant of Peptide F.

In some embodiments (e.g., certain embodiments in which a C-terminal carboxyl group of the protected variant of Fragment 2 is pre-activated), the method comprises:
contacting the protected variant of Fragment 2 with a coupling composition to form a protected variant of Fragment 2 with an activated C-terminal carboxyl group (“activated Fragment 2”); and
contacting the protected variant of Peptide E with the activated Fragment 2 to form the protected variant of Peptide F, wherein the contacting does not occur in the presence of a coupling reagent.

In some embodiments, the protected variant of Fragment 2 is present in molar excess relative to the coupling reagent in the coupling composition during the contacting of the protected variant of Fragment 2 and the coupling composition.

In some embodiments (e.g., certain embodiments in which a C-terminal carboxyl group of the protected variant of Fragment 2 is pre-activated), the method comprises:
contacting the protected variant of Fragment 2 with a coupling composition on solid support to form a protected variant of Fragment 2 with an activated C-terminal carboxyl group (“activated Fragment 2”); and
contacting the protected variant of Peptide E with the activated Fragment 2 on solid support to form the protected variant of Peptide F, wherein the contacting does not occur in the presence of a coupling reagent.

In some embodiments, the protected variant of Fragment 2 is present in molar excess relative to the coupling reagent in the coupling composition during the contacting of the protected variant of Fragment 2 and the coupling composition.

In some embodiments (e.g., certain embodiments in which a C-terminal carboxyl group of the protected variant of Fragment 2 is pre-activated), the method comprises:
contacting the protected variant of Fragment 2 with a coupling composition in solution to form a protected variant of Fragment 2 with an activated C-terminal carboxyl group (“activated Fragment 2”); and
contacting the protected variant of Peptide E with the activated Fragment 2 in solution to form the protected variant of Peptide F, wherein the contacting does not occur in the presence of a coupling reagent.

In some embodiments, the protected variant of Fragment 2 is present in molar excess relative to the coupling reagent in the coupling composition during the contacting of the protected variant of Fragment 2 and the coupling composition.

In some embodiments, the contacting of the protected variant of Fragment 2 with the coupling composition and the contacting of the activated Fragment 2 with the protected variant of Peptide E occur in the same reaction vessel. In other embodiments, the contacting of the protected variant of Fragment 2 with the coupling composition and the contacting of the activated Fragment 2 with the protected variant of Peptide E occur in different reaction vessels.

In some embodiments, the activated Fragment 2 is contacted with the protected variant of Peptide E in the presence of a base and/or a coupling additive but not a coupling reagent (such as, e.g., in certain embodiments in which a C-terminal carboxyl group of the protected variant of Fragment 2 is pre-activated and the protected variant of Fragment 2 is present in molar excess relative to the coupling reagent in the coupling composition during the contacting to form activated Fragment 2).

In some embodiments in which a C-terminal carboxyl group of the protected variant of Fragment 2 is pre-activated, the protected variant of Peptide E has a free N-terminal amino group and a free C-terminal carboxyl group.

In some embodiments, the coupling composition comprises a coupling reagent and a base. In some embodiments, the coupling composition comprises a coupling reagent (e.g., a carbodiimide, e.g., DIC, DCC, EDC) and a coupling additive (e.g., HOBt, 5-Cl—HOBt, Oxyma).

In some embodiments, the coupling composition comprises a coupling reagent, a coupling additive, and a base.

In some embodiments, the coupling reagent is a carbodiimide, such as, e.g., DIC, DCC, or EDC. In other embodiments, the coupling reagent is BOP—Cl.

In some embodiments (e.g., certain embodiments in which a C-terminal carboxyl group of the protected variant of Fragment 2 is activated in situ), the method comprises coupling the protected variant of Fragment 2 with the protected variant of Peptide E in the presence of a coupling composition. In some embodiments, the method comprises coupling the protected variant of Fragment 2 with the protected variant of Peptide E in solution in the presence of a coupling composition.

In some embodiments, the protected variant of Peptide E does not have a free carboxyl group at the C-terminus. In some embodiments, the C-terminus of the protected variant of Peptide E is protected. In some embodiments, the C-terminus of the protected variant of Peptide E is amidated. In some embodiments, the protected variant of Peptide E has a free amino group at the N-terminus. In preferred embodiments, the protected variant of Peptide E has a free amino group at the N-terminus and does not have a free carboxyl group at the C-terminus. In particularly preferred embodiments, the protected variant of Peptide E has a free amino group at the N-terminus and an amidated C-terminus.

In some embodiments, the protected variant of Fragment 2 does not have a free amino group at the N-terminus. In some embodiments, the N-terminus of the protected variant of Fragment 2 is protected (preferably Boc-protected). In some embodiments, the protected variant of Fragment 2 has a free carboxyl group at the C-terminus. In preferred embodiments, the protected variant of Fragment 2 has a free carboxyl group at the C-terminus and does not have a free amino group at the N-terminus. In particularly preferred embodiments, the protected variant of Fragment 2 has a free carboxyl group at the C-terminus and a protected (preferably Boc-protected) N-terminus.

In some embodiments, the protected variant of Peptide E has a free amino group at the N-terminus and does not have a free carboxyl group at the C-terminus, and the protected variant of Fragment 2 has a free carboxyl group at the C-terminus and a protected (preferably Boc-protected) N-terminus.

In some embodiments, the C-terminus of the protected variant of Peptide E is amidated, and the N-terminus of the protected variant of Fragment 2 is protected (preferably Boc-protected).

In preferred embodiments, the protected variant of Peptide E has a free amino group at the N-terminus and does not have a free carboxyl group at the C-terminus, and the protected variant of Fragment 2 has a free carboxyl group at the C-terminus and does not have a free amino group at the N-terminus. In particularly preferred embodiments, the protected variant of Peptide E has a free amino group at the N-terminus and an amidated C-terminus, and the protected variant of Fragment 2 has a free carboxyl group at the C-terminus and a protected (preferably Boc-protected) N-terminus.

In some embodiments, the protected variant of Peptide E has a free amino group at the N-terminus and an amidated C-terminus, and the protected variant of Fragment 2 has a free carboxyl group at the C-terminus and a Boc-protected N-terminus.

In some embodiments, the protected variant of Fragment 2 is Fmoc-Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 52).

In some embodiments, the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 160).

In some embodiments, the protected variant of Fragment 2 is Fmoc-Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 52), and the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 160).

In some embodiments, the coupling composition comprises TBTU/collidine, PyBOP/TEA, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the coupling composition comprises DIC/Oxyma or TBTU/collidine. In some embodiments, the coupling composition comprises DIC/Oxyma. In some embodiments, the coupling composition comprises TBTU/collidine.

In some embodiments, the coupling composition comprises DIC and Oxyma. In some embodiments, the coupling composition comprises DIC and Oxyma in DMSO.

In some embodiments, the coupling composition comprises TBTU and collidine. In some embodiments, the coupling composition comprises TBTU and collidine in DMSO.

In some embodiments, the protected variant of Fragment 2 is Fmoc-Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 52), the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 160), and the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the protected variant of Fragment 2 is Fmoc-Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 52), the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 160), and the coupling composition comprises DIC/Oxyma or TBTU/collidine (preferably DIC/Oxyma). In some embodiments, the coupling composition comprises DIC/Oxyma or TBTU/collidine in DMSO. In some embodiments, the coupling composition comprises DIC and Oxyma in DMSO. In some embodiments, the coupling composition comprises TBTU and collidine in DMSO.

In some embodiments, the protected variant of Fragment 2 is Fmoc-Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 52), and the coupling composition comprises DIC and Oxyma. In some embodiments, the coupling composition comprises DIC and Oxyma in DMSO.

In some embodiments, the protected variant of Fragment 2 is Fmoc-Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 52), and the coupling composition comprises TBTU and collidine. In some embodiments, the coupling composition comprises TBTU and collidine in DMSO.

In some embodiments, the coupling occurs at a temperature in the range of 0° C. to 60° C. In some embodiments, the coupling occurs at a temperature in the range of 20° C. to 50° C. In some embodiments, the coupling occurs at a temperature in the range of 20° C. to 30° C. In some embodiments, the coupling occurs at a temperature in the range of 30° C. to 50° C. In some embodiments, the coupling occurs at a temperature in the range of 35° C. to 45° C.

In some embodiments, the method comprises coupling the protected variant of Fragment 2 and the protected variant of Peptide E for a time in the range of 1 hour to 72 hours. In some embodiments, the time is in the range of 6 hours to 24 hours.

In some embodiments, the coupling occurs at a temperature in the range of 0° C. to 60° C. for a time in the range of 1 hour to 72 hours. In some embodiments, the coupling occurs at a temperature in the range of 0° C. to 60° C. for a time in the range of 6 hours to 24 hours. In some embodiments, the coupling occurs at a temperature in the range of 20° C. to 50° C. for a time in the range of 1 hour to 72 hours. In some embodiments, the coupling occurs at a temperature in the range of 20° C. to 50° C. for a time in the range of 6 hours to 24 hours.

In some embodiments, the protected variant of Fragment 2 is Fmoc-Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 52), the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 160), the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine, and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours).

In some embodiments, the protected variant of Fragment 2 is Fmoc-Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 52), the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 160), the coupling composition comprises DIC/Oxyma or TBTU/collidine (preferably DIC/Oxyma), and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours). In some embodiments, the coupling composition comprises DIC and Oxyma in DMSO.

Also provided herein is a method of preparing Peptide A or a protected variant thereof, comprising coupling a protected variant of Fragment 1 with a protected variant of Peptide F to form a protected variant of Peptide A, wherein Fragment 1 comprises the amino acid sequence of SEQ ID NO: 1, Peptide F comprises the amino acid sequence of SEQ ID NO: 14, and Peptide A comprises the amino acid sequence of SEQ ID NO: 7. In some embodiments, the method further comprises deprotecting the protected variant of Peptide A to isolate Peptide A. In some embodiments, the method further comprises adding a bromoacetyl moiety to the Lys at the C-terminus of Peptide A or a protected variant thereof.

In some embodiments, the protected variant of Fragment 1 is coupled with the protected variant of Peptide F in solution. In some embodiments, the protected variant of Fragment 1 is coupled with the protected variant of Peptide F under liquid phase peptide coupling conditions. In some embodiments, the protected variant of Fragment 1 is coupled with the protected variant of Peptide F under classical solution phase peptide coupling conditions. In some embodiments, the protected variant of Fragment 1 is coupled with the protected variant of Peptide F under tag-assisted liquid phase peptide coupling conditions.

In other embodiments, the protected variant of Fragment 1 is coupled with the protected variant of Peptide F on solid support. In some embodiments, the protected variant of Fragment 1 is coupled with the protected variant of Peptide F under solid phase peptide coupling conditions.

In some embodiments, a C-terminal carboxyl group of the protected variant of Fragment 1 is pre-activated. In other embodiments, a C-terminal carboxyl group of the protected variant of Fragment 1 is activated in situ.

In some embodiments, the method comprises:
contacting the protected variant of Fragment 1 with a coupling composition to form a protected variant of Fragment 1 with an activated C-terminal carboxyl group (“activated Fragment 1”); and
contacting the protected variant of Peptide F with the activated Fragment 1 to form the protected variant of Peptide A.

In some embodiments, the method comprises:
contacting the protected variant of Fragment 1 with a coupling composition on solid support to form a protected variant of Fragment 1 with an activated C-terminal carboxyl group (“activated Fragment 1”); and
contacting the protected variant of Peptide F with the activated Fragment 1 on solid support to form the protected variant of Peptide A.

In some embodiments, the method comprises:
contacting the protected variant of Fragment 1 with a coupling composition in solution to form a protected variant of Fragment 1 with an activated C-terminal carboxyl group (“activated Fragment 1”); and
contacting the protected variant of Peptide F with the activated Fragment 1 in solution to form the protected variant of Peptide A.

In some embodiments (e.g., certain embodiments in which a C-terminal carboxyl group of the protected variant of Fragment 1 is pre-activated), the method comprises:
contacting the protected variant of Fragment 1 with a coupling composition to form a protected variant of Fragment 1 with an activated C-terminal carboxyl group (“activated Fragment 1”); and
contacting the protected variant of Peptide F with the activated Fragment 1 to form the protected variant of Peptide A, wherein the contacting does not occur in the presence of a coupling reagent.

In some embodiments, the protected variant of Fragment 1 is present in molar excess relative to the coupling reagent in the coupling composition during the contacting of the protected variant of Fragment 1 and the coupling composition.

In some embodiments (e.g., certain embodiments in which a C-terminal carboxyl group of the protected variant of Fragment 1 is pre-activated), the method comprises:
contacting the protected variant of Fragment 1 with a coupling composition on solid support to form a protected variant of Fragment 1 with an activated C-terminal carboxyl group (“activated Fragment 1”); and
contacting the protected variant of Peptide F with the activated Fragment 1 on solid support to form the protected variant of Peptide A, wherein the contacting does not occur in the presence of a coupling reagent.

In some embodiments, the protected variant of Fragment 1 is present in molar excess relative to the coupling reagent in the coupling composition during the contacting of the protected variant of Fragment 1 and the coupling composition.

In some embodiments (e.g., certain embodiments in which a C-terminal carboxyl group of the protected variant of Fragment 1 is pre-activated), the method comprises:
contacting the protected variant of Fragment 1 with a coupling composition in solution to form a protected variant of Fragment 1 with an activated C-terminal carboxyl group (“activated Fragment 1”); and
contacting the protected variant of Peptide F with the activated Fragment 1 in solution to form the protected variant of Peptide A, wherein the contacting does not occur in the presence of a coupling reagent.

In some embodiments, the protected variant of Fragment 1 is present in molar excess relative to the coupling reagent in the coupling composition during the contacting of the protected variant of Fragment 1 and the coupling composition.

In some embodiments, the contacting of the protected variant of Fragment 1 with the coupling composition and the contacting of the activated Fragment 1 with the protected variant of Peptide F occur in the same reaction vessel. In other embodiments, the contacting of the protected variant of Fragment 1 with the coupling composition and the contacting of the activated Fragment 1 with the protected variant of Peptide F occur in different reaction vessels.

In some embodiments, the activated Fragment 1 is contacted with the protected variant of Peptide F in the presence of a base and/or a coupling additive but not a coupling reagent (such as, e.g., in certain embodiments in which a C-terminal carboxyl group of the protected variant of Fragment 1 is pre-activated and the protected variant of Fragment 1 is present in molar excess relative to the coupling reagent in the coupling composition during the contacting to form activated Fragment 1).

In some embodiments in which a C-terminal carboxyl group of the protected variant of Fragment 1 is pre-activated, the protected variant of Peptide F has a free N-terminal amino group and a free C-terminal carboxyl group.

In some embodiments, the coupling composition comprises a coupling reagent and a base. In some embodiments, the coupling composition comprises a coupling reagent (e.g., a carbodiimide, e.g., DIC, DCC, EDC) and a coupling additive (e.g., HOBt, 5-Cl—HOBt, Oxyma).

In some embodiments, the coupling composition comprises a coupling reagent, a coupling additive, and a base.

In some embodiments, the coupling reagent is a carbodiimide, such as, e.g., DIC, DCC, or EDC. In other embodiments, the coupling reagent is BOP—Cl.

In some embodiments (e.g., certain embodiments in which a C-terminal carboxyl group of the protected variant of Fragment 1 is activated in situ), the method comprises coupling the protected variant of Fragment 1 with the protected variant of Peptide F in the presence of a coupling composition. In some embodiments, the method comprises coupling the protected variant of Fragment 1 with the protected variant of Peptide F in solution in the presence of a coupling composition.

In some embodiments, the protected variant of Peptide F does not have a free carboxyl group at the C-terminus. In some embodiments, the C-terminus of the protected variant of Peptide F is protected. In some embodiments, the C-terminus of the protected variant of Peptide F is amidated. In some embodiments, the protected variant of Peptide F has a free amino group at the N-terminus. In preferred embodiments, the protected variant of Peptide F has a free amino group at the N-terminus and does not have a free carboxyl group at the C-terminus. In particularly preferred embodiments, the protected variant of Peptide F has a free amino group at the N-terminus and an amidated C-terminus.

In some embodiments, the protected variant of Fragment 1 does not have a free amino group at the N-terminus. In some embodiments, the N-terminus of the protected variant of Fragment 1 is protected (preferably Boc-protected). In some embodiments, the protected variant of Fragment 1 has a free carboxyl group at the C-terminus. In preferred embodiments, the protected variant of Fragment 1 has a free carboxyl group at the C-terminus and does not have a free amino group at the N-terminus. In particularly preferred embodiments, the protected variant of Fragment 1 has a free carboxyl group at the C-terminus and a protected (preferably Boc-protected) N-terminus.

In some embodiments, the protected variant of Peptide F has a free amino group at the N-terminus and does not have a free carboxyl group at the C-terminus, and the protected variant of Fragment 1 has a free carboxyl group at the C-terminus and a protected (preferably Boc-protected) N-terminus.

In some embodiments, the C-terminus of the protected variant of Peptide F is amidated, and the N-terminus of the protected variant of Fragment 1 is protected (preferably Boc-protected).

In preferred embodiments, the protected variant of Peptide F has a free amino group at the N-terminus and does not have a free carboxyl group at the C-terminus, and the protected variant of Fragment 1 has a free carboxyl group at the C-terminus and does not have a free amino group at the N-terminus. In particularly preferred embodiments, the protected variant of Peptide F has a free amino group at the N-terminus and an amidated C-terminus, and the protected variant of Fragment 1 has a free carboxyl group at the C-terminus and a protected (preferably Boc-protected) N-terminus.

In some embodiments, the protected variant of Peptide F has a free amino group at the N-terminus and an amidated C-terminus, and the protected variant of Fragment 1 has a free carboxyl group at the C-terminus and a Boc-protected N-terminus.

In some embodiments, the protected variant of Fragment 1 is Boc-His(Trt)-Aib-Glu(OtBu)-Gly-Thr(tBu)-Phe-Thr(tBu)-Ser(tBu)-Asp (OMpe)-Tyr(tBu)-Ser(tBu)-Ser(Psi(Me,Me)Pro)-OH (SEQ ID NO: 84).

In some embodiments, the protected variant of Peptide F is Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 85).

In some embodiments, the protected variant of Fragment 1 is Boc-His(Trt)-Aib-Glu(OtBu)-Gly-Thr(tBu)-Phe-Thr(tBu)-Ser(tBu)-Asp (OMpe)-Tyr(tBu)-Ser(tBu)-Ser(Psi(Me,Me)Pro)-OH (SEQ ID NO: 84), and the protected variant of Peptide F is Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 85).

In some embodiments, the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the coupling composition comprises DIC/Oxyma, EDC/Oxyma, PyBOP/collidine, PyOxim/collidine, or TBTU/collidine. In some embodiments, the coupling composition.

In some embodiments, the coupling composition comprises DIC and Oxyma. In some embodiments, the coupling composition comprises DIC and Oxyma in DMSO. In some embodiments, the coupling composition comprises DIC and Oxyma in DMF.

In some embodiments, the protected variant of Fragment 1 is Boc-His(Trt)-Aib-Glu(OtBu)-Gly-Thr(tBu)-Phe-Thr(tBu)-Ser(tBu)-Asp (OMpe)-Tyr(tBu)-Ser(tBu)-Ser(Psi(Me,Me)Pro)-OH (SEQ ID NO: 84), the protected variant of Peptide F is Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 85), and the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine.

In some embodiments, the protected variant of Fragment 1 is Boc-His(Trt)-Aib-Glu(OtBu)-Gly-Thr(tBu)-Phe-Thr(tBu)-Ser(tBu)-Asp (OMpe)-Tyr(tBu)-Ser(tBu)-Ser(Psi(Me,Me)Pro)-OH (SEQ ID NO: 84), the protected variant of Peptide F is Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 85), and the coupling composition comprises DIC/Oxyma, EDC/Oxyma, PyBOP/collidine, PyOxim/collidine, or TBTU/collidine (preferably DIC/Oxyma). In some embodiments, the coupling composition comprises DIC/Oxyma, EDC/Oxyma, PyBOP/collidine, PyOxim/collidine, or TBTU/collidine (preferably DIC/Oxyma) in DMSO. In some embodiments, the coupling composition comprises DIC and Oxyma in DMSO or DMF. In some embodiments, the coupling composition comprises DIC and Oxyma in DMSO. In some embodiments, the coupling composition comprises DIC and Oxyma in DMF.

In some embodiments, the protected variant of Fragment 1 is Boc-His(Trt)-Aib-Glu(OtBu)-Gly-Thr(tBu)-Phe-Thr(tBu)-Ser(tBu)-Asp (OMpe)-Tyr(tBu)-Ser(tBu)-Ser(Psi(Me,Me)Pro)-OH (SEQ ID NO: 84), and the coupling composition comprises DIC and Oxyma. In some embodiments, the coupling composition comprises DIC and Oxyma in DMSO or DMF. In some embodiments, the coupling composition comprises DIC and Oxyma in DMSO. In some embodiments, the coupling composition comprises DIC and Oxyma in DMF.

In some embodiments, the coupling occurs at a temperature in the range of 0° C. to 60° C. In some embodiments, the coupling occurs at a temperature in the range of 20° C. to 50° C. In some embodiments, the coupling occurs at a temperature in the range of 20° C. to 30° C. In some embodiments, the coupling occurs at a temperature in the range of 30° C. to 50° C. In some embodiments, the coupling occurs at a temperature in the range of 35° C. to 45° C.

In some embodiments, the method comprises coupling the protected variant of Fragment 1 and the protected variant of Peptide F for a time in the range of 1 hour to 72 hours. In some embodiments, the time is in the range of 6 hours to 24 hours.

In some embodiments, the coupling occurs at a temperature in the range of 0° C. to 60° C. for a time in the range of 1 hour to 72 hours. In some embodiments, the coupling occurs at a temperature in the range of 0° C. to 60° C. for a time in the range of 6 hours to 24 hours. In some embodiments, the coupling occurs at a temperature in the range of 20° C. to 50° C. for a time in the range of 1 hour to 72 hours. In some embodiments, the coupling occurs at a temperature in the range of 20° C. to 50° C. for a time in the range of 6 hours to 24 hours.

In some embodiments, the protected variant of Fragment 1 is Boc-His(Trt)-Aib-Glu(OtBu)-Gly-Thr(tBu)-Phe-Thr(tBu)-Ser(tBu)-Asp (OMpe)-Tyr(tBu)-Ser(tBu)-Ser(Psi(Me,Me)Pro)-OH (SEQ ID NO: 84), the protected variant of Peptide F is Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 85), the coupling composition comprises TBTU/collidine, PyBOP/TEA, TBTU/TMEDA, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, or Oxyma/PyOxim/collidine, and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours).

In some embodiments, the protected variant of Fragment 1 is Boc-His(Trt)-Aib-Glu(OtBu)-Gly-Thr(tBu)-Phe-Thr(tBu)-Ser(tBu)-Asp (OMpe)-Tyr(tBu)-Ser(tBu)-Ser(Psi(Me,Me)Pro)-OH (SEQ ID NO: 84), the protected variant of Peptide F is Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 85), the coupling composition comprises DIC/Oxyma, EDC/Oxyma, PyBOP/collidine, PyOxim/collidine, or TBTU/collidine (preferably DIC/Oxyma), and the coupling occurs at a temperature in the range of 0° C. to 60° C. (preferably a temperature in the range of 20° C. to 50° C.) for a time in the range of 1 hour to 72 hours (preferably a time in the range of 6 hours to 24 hours). In some embodiments, the coupling composition comprises DIC and Oxyma in DMSO or DMF. In some embodiments, the coupling composition comprises DIC and Oxyma in DMSO. In some embodiments, the coupling composition comprises DIC and Oxyma in DMF.

Also provided herein is a method of preparing Peptide A or a protected variant thereof, comprising:
coupling or having coupled a protected variant of Fragment 2 with a protected variant of Peptide E to form a first protected variant of Peptide F, wherein the protected variant of Fragment 2 has a free carboxyl group at the C-terminus and a protected (preferably Fmoc-protected) N-terminus, the protected variant of Peptide E has a free amino group at the N-terminus, Fragment 2 comprises the amino acid sequence of SEQ ID NO: 2, Peptide E comprises the amino acid sequence of SEQ ID NO: 8, and Peptide F comprises the amino acid sequence of SEQ ID NO: 14;
selectively deprotecting or having selectively deprotected the N-terminus of the first protected variant of Peptide F to form a second protected variant of Peptide F with a free amino group at the N-terminus;
coupling or having coupled a protected variant of Fragment 1 with the second protected variant of Peptide F to form a protected variant of Peptide A, wherein the protected variant of Fragment 1 has a free carboxyl group at the C-terminus and a protected (preferably Boc-protected) N-terminus, Fragment 1 comprises the amino acid sequence of SEQ ID NO: 1, and Peptide A comprises the amino acid sequence of SEQ ID NO: 7.

In some embodiments, the method further comprises deprotecting or having deprotected the protected variant of Peptide A to isolate Peptide A.

In some embodiments, the method further comprises adding or having added a bromoacetyl moiety to the Lys at the C-terminus of Peptide A or a protected variant thereof.

In some embodiments, the protected variant of Fragment 2 and the protected variant of Peptide E are coupled under conditions described herein.

In some embodiments, the protected variant of Fragment 1 and the second protected variant of Peptide F are coupled under conditions described herein.

In some embodiments, the protected variant of Fragment 2 and the protected variant of Peptide E are coupled under conditions described above, and the protected variant of Fragment 1 and the second protected variant of Peptide F are coupled under conditions described herein.

In some embodiments, the N-terminus of the first protected variant of Peptide F is Fmoc-protected, and the selective deprotection comprises contacting the first protected variant of Peptide F with an amine base to selectively remove the Fmoc protecting group at the N-terminus. In some embodiments, the amine base is selected from piperidine, piperazine, and morpholine (e.g., 5-30% (v/v) piperidine, piperazine, or morpholine). In some embodiments, the amine base is piperidine. In some embodiments, the N-terminus of the first protected variant of Peptide F is Fmoc-protected, and the selective deprotection comprises contacting the first protected variant of Peptide F with piperidine in DMF, optionally in the presence of DBU.

In some embodiments, the N-terminus of the first protected variant of Peptide F is Boc-protected, and the selective deprotection comprises contacting the first protected variant of Peptide F with an acid (preferably an acid selected from TFA, HCl, and HBr) to selectively remove the Boc protecting group at the N-terminus. In some embodiments, the first protected variant of Peptide F is contacted with TFA (e.g., neat TFA or TFA in DCM, e.g., 20-50% (v/V) TFA in DCM). In some embodiments, the first protected variant of Peptide F is contacted with HCl (e.g., HCl in DCM, DCE, toluene, or dioxane, e.g., 3-4 M HCl in DCM or dioxane).

In some embodiments, the N-terminus of the first protected variant of Peptide F is Boc-protected, and the selective deprotection comprises contacting the first protected variant of Peptide F with an acid (preferably an acid selected from TFA, HCl, and HBr) in the presence of a scavenger. In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some preferred embodiments, the scavenger is a hydrosilane. In some particularly preferred embodiments, the scavenger is TIS. In some embodiments, the first protected variant of Peptide F is contacted with TFA (e.g., neat TFA or TFA in DCM, e.g., 20-50% (v/V) TFA in DCM) in the presence of a scavenger (preferably TIS). In some embodiments, the first protected variant of Peptide F is contacted with HCl (e.g., HCl in DCM, DCE, toluene, or dioxane, e.g., 3-4 M HCl in DCM or dioxane) in the presence of a scavenger.

In some embodiments, the N-terminus of the first protected variant of Peptide F is Trt-protected, and the selective deprotection comprises contacting the first protected variant of Peptide F with a Brønsted acid or a Lewis acid (preferably TFA, HCl, or dichloroacetic acid, most preferably TFA), optionally in the presence of a scavenger (preferably TIS), to selectively remove the trityl protecting group at the N-terminus. In some embodiments, the first protected variant of Peptide F is contacted with a Brønsted acid (preferably a dilute Brønsted acid). In some embodiments, the Brønsted acid is selected from TFA, HCl, and dichloroacetic acid (preferably dilute TFA, HCl, or dichloroacetic acid). In other embodiments, the first protected variant of Peptide F is contacted with a Lewis acid (preferably a dilute Lewis acid).

In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some embodiments, the scavenger is a hydrosilane. In some embodiments, the scavenger is TIS.

In some embodiments, the N-terminus of the first protected variant of Peptide F is Trt-protected, and the selective deprotection comprises contacting the first protected variant of Peptide F with HCl. In some embodiments, the selective deprotection comprises contacting the first protected variant of Peptide F with dilute HCl.

In some embodiments, the N-terminus of the first protected variant of Peptide F is Trt-protected, and the selective deprotection comprises contacting the first protected variant of Peptide F with TFA in the presence of TIS. In some embodiments, the selective deprotection comprises contacting the first protected variant of Peptide F with dilute TFA in the presence of TIS.

Also provided herein is a method of preparing Peptide A or a protected variant thereof, comprising:
coupling or having coupled a protected variant of Fragment 3 with a protected variant of Fragment 4 to form a first protected variant of Peptide E, wherein the protected variant of Fragment 3 has a free carboxyl group at the C-terminus and a protected (preferably Fmoc-protected) N-terminus, the protected variant of Fragment 4 has a free amino group at the N-terminus, Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8;
selectively deprotecting or having selectively deprotected the N-terminus of the first protected variant of Peptide E to form a second protected variant of Peptide E with a free amino group at the N-terminus;
coupling or having coupled a protected variant of Fragment 2 with the second protected variant of Peptide E to form a first protected variant of Peptide F, wherein the protected variant of Fragment 2 has a free carboxyl group at the C-terminus and a protected (preferably Fmoc-protected) N-terminus, Fragment 2 comprises the amino acid sequence of SEQ ID NO: 2, and Peptide F comprises the amino acid sequence of SEQ ID NO: 14;
selectively deprotecting or having selectively deprotected the N-terminus of the first protected variant of Peptide F to form a second protected variant of Peptide F with a free amino group at the N-terminus; and
coupling or having coupled a protected variant of Fragment 1 with the second protected variant of Peptide F to form a protected variant of Peptide A, wherein the protected variant of Fragment 1 has a free carboxyl group at the C-terminus and a protected (preferably Boc-protected) N-terminus, Fragment 1 comprises the amino acid sequence of SEQ ID NO: 1, and Peptide A comprises the amino acid sequence of SEQ ID NO: 7.

In some embodiments, the method further comprises deprotecting or having deprotected the protected variant of Peptide A to isolate Peptide A.

In some embodiments, the method further comprises adding or having added a bromoacetyl moiety to the Lys at the C-terminus of Peptide A or a protected variant thereof.

In some embodiments, the protected variant of Fragment 3 and the protected variant of Fragment 4 are coupled under conditions described herein.

In some embodiments, the protected variant of Fragment 2 and the second protected variant of Peptide E are coupled under conditions described herein.

In some embodiments, the protected variant of Fragment 1 and the second protected variant of Peptide F are coupled under conditions described herein.

In some embodiments, the protected variant of Fragment 3 and the protected variant of Fragment 4 are coupled under conditions described herein, the protected variant of Fragment 2 and the second protected variant of Peptide E are coupled under conditions described herein, and the protected variant of Fragment 1 and the second protected variant of Peptide F are coupled under conditions described herein. In some embodiments, each coupling is performed under liquid phase peptide coupling conditions. In other embodiments, each coupling is performed under solid phase peptide coupling conditions.

In some embodiments, the N-termini of the first protected variant of Peptide E and the first protected variant of Peptide F are Fmoc-protected (“Fmoc-protected variants”), and each selective deprotection independently comprises contacting the Fmoc-protected variant with an amine base to selectively remove the Fmoc protecting group at the N-terminus. In some embodiments, the amine base is selected from piperidine, piperazine, and morpholine (e.g., 5-30% (v/v) piperidine, piperazine, or morpholine). In some embodiments, the amine base is piperidine. In some embodiments, each selective deprotection comprises contacting the Fmoc-protected variant with piperidine in DMF, optionally in the presence of DBU.

In some embodiments, the N-termini of the first protected variant of Peptide E and the first protected variant of Peptide F are Boc-protected (“Boc-protected variants”), and each selective deprotection independently comprises contacting the Boc-protected variant with an acid (preferably an acid selected from TFA, HCl, and HBr) to selectively remove the Boc protecting group at the N-terminus. In some embodiments, the Boc-protected variant is contacted with TFA (e.g., neat TFA or TFA in DCM, e.g., 20-50% (v/v) TFA in DCM). In some embodiments, the Boc-protected variant is contacted with HCl (e.g., HCl in DCM, DCE, toluene, or dioxane, e.g., 3-4 M HCl in DCM or dioxane).

In some embodiments, each selective deprotection independently comprises contacting the Boc-protected variant with an acid (preferably an acid selected from TFA, HCl, and HBr) in the presence of a scavenger. In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some preferred embodiments, the scavenger is a hydrosilane. In some particularly preferred embodiments, the scavenger is TIS. In some embodiments, the Boc-protected variant is contacted with TFA (e.g., neat TFA or TFA in DCM, e.g., 20-50% (v/V) TFA in DCM) in the presence of a scavenger (preferably TIS). In some embodiments, the Boc-protected variant is contacted with HCl (e.g., HCl in DCM, DCE, toluene, or dioxane, e.g., 3-4 M HCl in DCM or dioxane) in the presence of a scavenger.

In some embodiments, the N-termini of the first protected variant of Peptide E and the first protected variant of Peptide F are Trt-protected (“Trt-protected variants”), and each selective deprotection comprises contacting the Trt-protected variant with a Brønsted acid or a Lewis acid (preferably TFA, HCl, or dichloroacetic acid, most preferably TFA), optionally in the presence of a scavenger (preferably TIS), to selectively remove the trityl protecting group at the N-terminus. In some embodiments, the Trt-protected variant is contacted with a Brønsted acid (preferably a dilute Brønsted acid). In some embodiments, the Brønsted acid is selected from TFA, HCl, and dichloroacetic acid (preferably dilute TFA, HCl, or dichloroacetic acid). In other embodiments, the Trt-protected variant is contacted with a Lewis acid (preferably a dilute Lewis acid).

In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some embodiments, the scavenger is a hydrosilane. In some embodiments, the scavenger is TIS.

In some embodiments, each selective deprotection comprises contacting the Trt-protected variant with HCl. In some embodiments, each selective deprotection comprises contacting the Trt-protected variant with dilute HCl.

In some embodiments, each selective deprotection comprises contacting the Trt-protected variant with TFA in the presence of TIS. In some embodiments, each selective deprotection comprises contacting the Trt-protected variant with dilute TFA in the presence of TIS.

Also provided herein is a method of preparing Peptide A or a protected variant thereof, comprising:
coupling or having coupled a protected variant of Fragment 3 with a protected variant of Fragment 4 to form a first protected variant of Peptide E, wherein the protected variant of Fragment 3 has a free carboxyl group at the C-terminus and a protected (preferably Fmoc-protected) N-terminus, the protected variant of Fragment 4 has a free amino group at the N-terminus, Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8;

selectively deprotecting or having selectively deprotected the N-terminus of the first protected variant of Peptide E to form a second protected variant of Peptide E with a free amino group at the N-terminus;
coupling or having coupled a protected variant of Fragment 2C with the second protected variant of Peptide E to form a first protected variant of Peptide C, wherein the protected variant of Fragment 2C has a free carboxyl group at the C-terminus and a protected (preferably Fmoc-protected) N-terminus, Fragment 2C comprises the amino acid sequence of SEQ ID NO: 17, and Peptide C comprises the amino acid sequence of SEQ ID NO: 10;

selectively deprotecting or having selectively deprotected the N-terminus of the first protected variant of Peptide C to form a second protected variant of Peptide C with a free amino group at the N-terminus; and
coupling or having coupled a protected variant of Fragment 1A with the second protected variant of Peptide C to form a protected variant of Peptide A, wherein the protected variant of Fragment 1A has a free carboxyl group at the C-terminus and a protected (preferably Boc-protected) N-terminus, Fragment 1A comprises the amino acid sequence of SEQ ID NO: 11, and Peptide A comprises the amino acid sequence of SEQ ID NO: 7.

In some embodiments, the method further comprises deprotecting or having deprotected the protected variant of Peptide A to isolate Peptide A.

In some embodiments, the method further comprises adding or having added a bromoacetyl moiety to the Lys at the C-terminus of Peptide A or a protected variant thereof.

In some embodiments, the protected variant of Fragment 3 and the protected variant of Fragment 4 are coupled under conditions described herein.

In some embodiments, the protected variant of Fragment 2C and the second protected variant of Peptide E are coupled under conditions described herein.

In some embodiments, the protected variant of Fragment 1A and the second protected variant of Peptide C are coupled under conditions described herein.

In some embodiments, the protected variant of Fragment 3 and the protected variant of Fragment 4 are coupled under conditions described herein, the protected variant of Fragment 2 and the second protected variant of Peptide E are coupled under conditions described herein, and the protected variant of Fragment 1A and the second protected variant of Peptide C are coupled under conditions described herein. In some embodiments, each coupling is performed under liquid phase peptide coupling conditions. In other embodiments, each coupling is performed under solid phase peptide coupling conditions.

In some embodiments, the N-termini of the first protected variant of Peptide E and the first protected variant of Peptide C are Fmoc-protected (“Fmoc-protected variants”), and each selective deprotection independently comprises contacting the Fmoc-protected variant with an amine base to selectively remove the Fmoc protecting group at the N-terminus. In some embodiments, the amine base is selected from piperidine, piperazine, and morpholine (e.g., 5-30% (v/v) piperidine, piperazine, or morpholine). In some embodiments, the amine base is piperidine. In some embodiments, each selective deprotection comprises contacting the Fmoc-protected variant with piperidine in DMF, optionally in the presence of DBU.

In some embodiments, the N-termini of the first protected variant of Peptide E and the first protected variant of Peptide C are Boc-protected (“Boc-protected variants”), and each selective deprotection independently comprises contacting the Boc-protected variant with an acid (preferably an acid selected from TFA, HCl, and HBr) to selectively remove the Boc protecting group at the N-terminus. In some embodiments, the Boc-protected variant is contacted with TFA (e.g., neat TFA or TFA in DCM, e.g., 20-50% (v/v) TFA in DCM). In some embodiments, the Boc-protected variant is contacted with HCl (e.g., HCl in DCM, DCE, toluene, or dioxane, e.g., 3-4 M HCl in DCM or dioxane).

In some embodiments, each selective deprotection independently comprises contacting the Boc-protected variant with an acid (preferably an acid selected from TFA, HCl, and HBr) in the presence of a scavenger. In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some preferred embodiments, the scavenger is a hydrosilane. In some particularly preferred embodiments, the scavenger is TIS. In some embodiments, the Boc-protected variant is contacted with TFA (e.g., neat TFA or TFA in DCM, e.g., 20-50% (v/V) TFA in DCM) in the presence of a scavenger (preferably TIS). In some embodiments, the Boc-protected variant is contacted with HCl (e.g., HCl in DCM, DCE, toluene, or dioxane, e.g., 3-4 M HCl in DCM or dioxane) in the presence of a scavenger.

In some embodiments, the N-termini of the first protected variant of Peptide E and the first protected variant of Peptide C are Trt-protected (“Trt-protected variants”), and each selective deprotection comprises contacting the Trt-protected variant with a Brønsted acid or a Lewis acid (preferably TFA, HCl, or dichloroacetic acid, most preferably TFA), optionally in the presence of a scavenger (preferably TIS), to selectively remove the trityl protecting group at the N-terminus. In some embodiments, the Trt-protected variant is contacted with a Brønsted acid (preferably a dilute Brønsted acid). In some embodiments, the Brønsted acid is selected from TFA,
HCl, and dichloroacetic acid (preferably dilute TFA, HCl, or dichloroacetic acid). In other embodiments, the Trt-protected variant is contacted with a Lewis acid (preferably a dilute Lewis acid).

In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some embodiments, the scavenger is a hydrosilane. In some embodiments, the scavenger is TIS.

In some embodiments, each selective deprotection comprises contacting the Trt-protected variant with HCl. In some embodiments, each selective deprotection comprises contacting the Trt-protected variant with dilute HCl.

In some embodiments, each selective deprotection comprises contacting the Trt-protected variant with TFA in the presence of TIS. In some embodiments, each selective deprotection comprises contacting the Trt-protected variant with dilute TFA in the presence of TIS.

Also provided herein is a method of preparing Peptide A or a protected variant thereof, comprising: coupling or having coupled a protected variant of Fragment 3A with a protected variant of Fragment 4 to form a first protected variant of Peptide B, wherein the protected variant of Fragment 3A has a free carboxyl group at the C-terminus and a protected (preferably Fmoc-protected) N-terminus, the protected variant of Fragment 4 has a free amino group at the N-terminus, Fragment 3A comprises the amino acid sequence of SEQ ID NO: 13, Fragment 4 comprises the amino acid sequence of SEQ ID NO: 4, and Peptide B comprises the amino acid sequence of SEQ ID NO: 9;
selectively deprotecting or having selectively deprotected the N-terminus of the first protected variant of Peptide B to form a second protected variant of Peptide B with a free amino group at the N-terminus;
coupling or having coupled a protected variant of Fragment 2A with the second protected variant of Peptide B to form a first protected variant of Peptide C, wherein the protected variant of Fragment 2A has a free carboxyl group at the C-terminus and a protected (preferably Fmoc-protected) N-terminus, Fragment 2A comprises the amino acid sequence of SEQ ID NO: 12, and Peptide C comprises the amino acid sequence of SEQ ID NO: 10;
selectively deprotecting or having selectively deprotected the N-terminus of the first protected variant of Peptide C to form a second protected variant of Peptide C with a free amino group at the N-terminus; and
coupling or having coupled a protected variant of Fragment 1A with the second protected variant of Peptide C to form a protected variant of Peptide A, wherein the protected variant of Fragment 1A has a free carboxyl group at the C-terminus and a protected (preferably Boc-protected) N-terminus, Fragment 1A comprises the amino acid sequence of SEQ ID NO: 11, and Peptide A comprises the amino acid sequence of SEQ ID NO: 7.

In some embodiments, the method further comprises deprotecting or having deprotected the protected variant of Peptide A to isolate Peptide A.

In some embodiments, the method further comprises adding or having added a bromoacetyl moiety to the Lys at the C-terminus of Peptide A or a protected variant thereof.

In some embodiments, the protected variant of Fragment 3A and the protected variant of Fragment 4 are coupled under conditions described herein.

In some embodiments, the protected variant of Fragment 2A and the second protected variant of Peptide B are coupled under conditions described herein.

In some embodiments, the protected variant of Fragment 1A and the second protected variant of Peptide C are coupled under conditions described herein.

In some embodiments, the protected variant of Fragment 3A and the protected variant of Fragment 4 are coupled under conditions described herein, the protected variant of Fragment 2A and the second protected variant of Peptide B are coupled under conditions described herein, and the protected variant of Fragment 1A and the second protected variant of Peptide C are coupled under conditions described herein. In some embodiments, each coupling is performed under liquid phase peptide coupling conditions. In other embodiments, each coupling is performed under solid phase peptide coupling conditions.

In some embodiments, the N-termini of the first protected variant of Peptide B and the first protected variant of Peptide C are Fmoc-protected (“Fmoc-protected variants”), and each selective deprotection independently comprises contacting the Fmoc-protected variant with an amine base to selectively remove the Fmoc protecting group at the N-terminus. In some embodiments, the amine base is selected from piperidine, piperazine, and morpholine (e.g., 5-30% (v/v) piperidine, piperazine, or morpholine). In some embodiments, the amine base is piperidine. In some embodiments, each selective deprotection comprises contacting the Fmoc-protected variant with piperidine in DMF, optionally in the presence of DBU.

In some embodiments, the N-termini of the first protected variant of Peptide B and the first protected variant of Peptide C are Boc-protected (“Boc-protected variants”), and each selective deprotection independently comprises contacting the Boc-protected variant with an acid (preferably an acid selected from TFA, HCl, and HBr) to selectively remove the Boc protecting group at the N-terminus. In some embodiments, the Boc-protected variant is contacted with TFA (e.g., neat TFA or TFA in DCM, e.g., 20-50% (v/v) TFA in DCM). In some embodiments, the Boc-protected variant is contacted with HCl (e.g., HCl in DCM, DCE, toluene, or dioxane, e.g., 3-4 M HCl in DCM or dioxane).

In some embodiments, each selective deprotection independently comprises contacting the Boc-protected variant with an acid (preferably an acid selected from TFA, HCl, and HBr) in the presence of a scavenger. In some embodiments, the scavenger is selected from hydrosilanes (e.g., TIS, triethylsilane (TES), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some preferred embodiments, the scavenger is a hydrosilane. In some particularly preferred embodiments, the scavenger is TIS. In some embodiments, the Boc-protected variant is contacted with TFA (e.g., neat TFA or TFA in DCM, e.g., 20-50% (v/V) TFA in DCM) in the presence of a scavenger (preferably TIS). In some embodiments, the Boc-protected variant is contacted with HCl (e.g., HCl in DCM, DCE, toluene, or dioxane, e.g., 3-4 M HCl in DCM or dioxane) in the presence of a scavenger.

In some embodiments, the N-termini of the first protected variant of Peptide B and the first protected variant of Peptide C are Trt-protected (“Trt-protected variants”), and each selective deprotection comprises contacting the Trt-protected variant with a Brønsted acid or a Lewis acid (preferably TFA, HCl, or dichloroacetic acid, most preferably TFA), optionally in the presence of a scavenger (preferably TIS), to selectively remove the trityl protecting group at the N-terminus. In some embodiments, the Trt-protected variant is contacted with a Brønsted acid (preferably a dilute Brønsted acid). In some embodiments, the Brønsted acid is selected from TFA, HCl, and dichloroacetic acid (preferably dilute TFA, HCl, or dichloroacetic acid). In other embodiments, the Trt-protected variant is contacted with a Lewis acid (preferably a dilute Lewis acid).

In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some embodiments, the scavenger is a hydrosilane. In some embodiments, the scavenger is TIS.

In some embodiments, each selective deprotection comprises contacting the Trt-protected variant with HCl. In some embodiments, each selective deprotection comprises contacting the Trt-protected variant with dilute HCl.

In some embodiments, each selective deprotection comprises contacting the Trt-protected variant with TFA in the presence of TIS. In some embodiments, each selective deprotection comprises contacting the Trt-protected variant with dilute TFA in the presence of TIS.

### Peptide Fragments

Convergent peptide synthesis approaches, including those described herein, assemble peptide sequences from shorter, pre-formed peptide fragment intermediates, which can improve synthetic efficiency and enable parallel manufacturing. Careful selection of fragments for use in a convergent peptide synthesis strategy, including choice of fragment length and junction sites, can impact these outcomes by reducing unwanted side reactions, influencing coupling efficiency, and enhancing both yield and purity. Additionally, protecting-group strategies at fragment termini and side chains can influence fragment solubility and susceptibility to side reactions. To develop efficient and scalable convergent approaches for the preparation of Peptide A and variants thereof (e.g., protected variants in which one or more amino acid side chains and/or termini are protected; a bromoacetylated variant of Peptide A in which the ε-amino group of the C-terminal Lys residue is bromoacetylated to enable conjugation to another molecule via a thiol-bromoacetyl reaction), various peptide fragments and protecting group strategies were investigated.

Peptide fragments and protected variants described herein may be isolated peptides or isolated protected variants, respectively. Additionally, in some embodiments, a peptide or protected variant thereof may be in the form of a salt. For example, in some embodiments, a peptide or protected variant thereof (e.g., an intended amine coupling partner) may be in the form of an acid-addition salt (e.g., a TFA, HCl, or acetate salt). In some embodiments, a peptide or protected variant thereof (e.g., an intended carboxyl coupling partner) may be in the form of a carboxylate salt (e.g., a sodium or triethylammonium salt).

Furthermore, in some peptide synthesis methods described herein, the amine coupling partner may initially be present in the form of an acid-addition salt, wherein the α-amino group is liberated for reaction with an activated carboxyl group through contact with a base (e.g., a base in a coupling composition). In addition, in some peptide synthesis methods described herein, the carboxyl coupling partner may initially be present in the form of a carboxylate salt, wherein the peptide or protected variant thereof is converted to a free acid and then activated for coupling.

### Fragment 1

Provided herein is a peptide having a sequence of His-Aib-Glu-Gly-Thr-Phe-Thr-Ser-Asp-Tyr-Ser-Ser (SEQ ID NO: 1) (“Fragment 1”). Certain aspects provide a protected variant of the peptide of Fragment 1, wherein at least one of the His, Glu, Thr, Ser, Asp, and Tyr residues is protected with a protecting group.

In some embodiments, the disclosure provides a protected variant of Fragment 1, wherein:
the side chain of the His residue at position 1 is protected with a trityl (Trt), tert-butyloxycarbonyl (Boc), mesitylene-2-sulfonyl (Mts), monomethoxytrityl (Mmt), or 4-methyltrityl (Mtt) protecting group; and/or the side chain of the Glu residue at position 3 is protected with a tert-butyl ester (OtBu), O-2-phenylisopropyl ester (OPp), or allyl ester (OAll) protecting group; and/or the side chain of the Thr residue at position 5 or position 7 is protected with a tert-butyl (tBu), O-trityl (OTrt), O-allyl (OAll), or pseudoproline (YPro) protecting group; and/or the side chain of the Ser residue at position 8, position 11, or position 12 is protected with tert-butyl (tBu), O-trityl (OTrt), O-allyl (OAll), or pseudoproline (YPro) protecting group; and/or the side chain of the Asp residue at position 9 is protected with a tert-butyl ester (OtBu), O-2-phenylisopropyl ester (OPp), or allyl ester (OAll) protecting group; and/or the side chain of the Tyr residue at position 10 is protected with a tert-butyl (tBu), O-allyl (OAll), trityl (Trt), or 2-chlorotrityl (Clt) protecting group.

In some embodiments, the N-terminus of the His residue at position 1 is protected with a 9-fluorenylmethoxycarbonyl (Fmoc) protecting group.

In some embodiments, the disclosure provides a protected variant of Fragment 1, wherein:
the side chain of the His residue at position 1 is protected with a trityl (Trt) protecting group; and/or
the side chain of the Glu residue at position 3 is protected with a tert-butyl ester (OtBu) protecting group; and/or
the side chain of the Thr residue at position 5 or position 7 is protected with a tert-butyl (tBu) or O-trityl (OTrt) protecting group; and/or
the side chain of the Ser residue at position 8, position 11, or position 12 is protected with a tert-butyl (tBu) or O-trityl (OTrt) protecting group; and/or
the side chain of the Asp residue at position 9 is protected with a tert-butyl ester (OtBu) protecting group; and/or
the side chain of the Tyr residue at position 10 is protected with a tert-butyl (tBu) protecting group.

In some embodiments, the N-terminus of the His residue at position 1 is protected with a 9-fluorenylmethoxycarbonyl (Fmoc) protecting group.

In some embodiments, the disclosure provides a protected variant of Fragment 1, wherein:
the side chain of the His residue at position 1 is protected with a 2,4-dinitrophenyl (Dnp), p-toluenesulfonyl (Tos), o-nitrobenzyl (ONb), or benzyloxymethyl (Bom) protecting group; and/or
the side chain of the Glu residue at position 3 is protected with an allyl ester (OAll), benzyl ester (OBn), or cyclohexyl ester (OcHex) protecting group; and/or
the side chain of the Thr residue at position 5 or position 7 is protected with a p-bromobenzyloxycarbonyl (Br-Z), O-benzyl (OBn), or O-allyl (OAll) protecting group; and/or
the side chain of the Ser residue at position 8, position 11, or position 12 is protected with a p-bromobenzyloxycarbonyl (Br-Z), O-benzyl (OBn), or O-allyl (OAll) protecting group; and/or
the side chain of the Asp residue at position 9 is protected with an allyl ester (OAll), benzyl ester (OBn), or cyclohexyl ester (OcHex) protecting group; and/or
the side chain of the Tyr residue at position 10 is protected with an O-benzyl (OBn), O-allyl (OAll), O-(2-bromobenzyl), or 2,6-dichlorobenzyl (Dcb) protecting group.

In some embodiments, the disclosure provides a protected variant of Fragment 1, wherein:
the side chain of the His residue at position 1 is protected with a p-toluenesulfonyl (Tos) protecting group; and/or
the side chain of the Glu residue at position 3 is protected with a benzyl ester (OBn) or allyl ester (OAll) protecting group; and/or
the side chain of the Thr residue at position 5 or position 7 is protected with an O-benzyl (OBn) protecting group; and/or
the side chain of the Ser residue at position 8, position 11, or position 12 is protected with an O-benzyl (OBn) protecting group; and/or
the side chain of the Asp residue at position 9 is protected with a benzyl ester (OBn) or allyl ester (OAll) protecting group; and/or
the side chain of the Tyr residue at position 10 is protected with an O-benzyl (OBn) protecting group.

In some embodiments, the N-terminus of the His residue at position 1 is protected with a tert-butyloxycarbonyl (Boc) protecting group.

Certain aspects provide a protected variant of Fragment 1, wherein the N-terminal His residue is protected with a Boc, Fmoc, or Trt protecting group. In some embodiments, the His residue is protected with a Boc or Fmoc protecting group. In some embodiments, the His residue is protected with a Trt protecting group. Certain aspects provide a protected variant of Fragment 1, wherein the N-terminal His is protected with a Boc protecting group. Certain aspects provide a protected variant of Fragment 1, wherein the Glu residue at position 3 is protected as a t-butyl ester (OtBu). Certain aspects provide a protected variant of Fragment 1, wherein the Thr residue at position 5 is protected as a t-butyl ether (tBu). Certain aspects provide a protected variant of Fragment 1, wherein the Thr residue at position 7 is protected as a t-butyl ether (tBu). Certain aspects provide a protected variant of Fragment 1, wherein the Ser residue at position 8 is protected as a t-butyl ether (tBu). Certain aspects provide a protected variant of Fragment 1, wherein the Ser residue at position 11 is protected as a t-butyl ether (tBu). Certain aspects provide a protected variant of Fragment 1, wherein the Ser residue at position 12 is protected as a pseudoproline moiety (psiMe, Mepro). Certain aspects provide a protected variant of Fragment 1, wherein the Asp residue at position 9 is protected as a t-butyl ester (OtBu). Certain aspects provide a protected variant of Fragment 1, wherein the Tyr residue at position 10 is protected as a t-butyl ether (tBu).

In some embodiments, the present disclosure provides a protected variant of Fragment 1, wherein:
the Glu residue at position 3 is protected as a t-butyl ester (OtBu); and/or
the Thr residue at position 5 is protected as a t-butyl ether (tBu); and/or
the Thr residue at position 7 is protected as a t-butyl ether (tBu); and/or
the Ser residue at position 8 is protected as a t-butyl ether (tBu); and/or
the Asp residue at position 9 is protected as a t-butyl ester (OtBu); and/or
the Tyr residue at position 10 is protected as a t-butyl ether (tBu); and/or
the Ser residue at position 11 is protected as a t-butyl ether (tBu); and/or
the Ser residue at position 12 is protected as a pseudoproline moiety.

In some embodiments, the His residue at position 1 is protected with a Boc or Trt protecting group. In some embodiments, the His residue at position 1 is protected with a Boc protecting group. In some embodiments, the His residue at position 1 is protected with a Trt protecting group. In some embodiments, the N-terminus of the His residue at position 1 is protected with a Boc or Fmoc protecting group. In some embodiments, the N-terminus of the His residue at position 1 is protected with a Boc protecting group. In some embodiments, the N-terminus of the His residue at position 1 is protected with a Fmoc protecting group.

In some embodiments, the present disclosure provides a protected variant of Fragment 1, wherein:
the Glu residue at position 3 is protected as a t-butyl ester (OtBu);
the Thr residue at position 5 is protected as a t-butyl ether (tBu);
the Thr residue at position 7 is protected as a t-butyl ether (tBu);
the Ser residue at position 8 is protected as a t-butyl ether (tBu);
the Asp residue at position 9 is protected as a t-butyl ester (OtBu);
the Tyr residue at position 10 is protected as a t-butyl ether (tBu);
the Ser residue at position 11 is protected as a t-butyl ether (tBu); and
the Ser residue at position 12 is protected as a pseudoproline moiety.

In some embodiments, the His residue at position 1 is protected with a Boc or Trt protecting group. In some embodiments, the His residue at position 1 is protected with a Boc protecting group. In some embodiments, the His residue at position 1 is protected with a Trt protecting group. In some embodiments, the N-terminus of the His residue at position 1 is protected with a Boc or Fmoc protecting group. In some embodiments, the N-terminus of the His residue at position 1 is protected with a Boc protecting group. In some embodiments, the N-terminus of the His residue at position 1 is protected with a Fmoc protecting group.

In some embodiments, the protected variant of Fragment 1 is His(Trt)-Aib-Glu(OtBu)-Gly-Thr(tBu)-Phe-Thr(tBu)-Ser(tBu)-Asp (OMpe)-Tyr(tBu)-Ser(tBu)-Ser(Psi(Me, Me)Pro)-OH (SEQ ID NO: 18) or a salt (e.g., a carboxylate salt) thereof.

In some embodiments, the protected variant of Fragment 1 is His(Trt)-Aib-Glu(OtBu)-Gly-Thr(tBu)-Phe-Thr(tBu)-Ser(tBu)-Asp (OMpe)-Tyr(tBu)-Ser(tBu)-Ser(Psi(Me,Me)Pro)-OH, wherein the protected variant of Fragment 1 has a protected N-terminus (SEQ ID NO: 23), or a salt (e.g., a carboxylate salt) thereof. In some embodiments, the protected variant of Fragment 1 is Boc-His(Trt)-Aib-Glu(OtBu)-Gly-Thr(tBu)-Phe-Thr(tBu)-Ser(tBu)-Asp (OMpe)-Tyr(tBu)-Ser(tBu)-Ser(Psi(Me,Me)Pro)-OH (SEQ ID NO: 84) or a salt (e.g., a carboxylate salt) thereof. In some embodiments, the protected variant of Fragment 1 is Fmoc-His(Trt)-Aib-Glu(OtBu)-Gly-Thr(tBu)-Phe-Thr(tBu)-Ser(tBu)-Asp (OMpe)-Tyr(tBu)-Ser(tBu)-Ser(Psi(Me,Me)Pro)-OH (SEQ ID NO: 92) or a salt (e.g., a carboxylate salt) thereof. In some embodiments, the protected variant of Fragment 1 is in the form of a carboxylate salt (e.g., a sodium or triethylammonium salt).

In some embodiments, the protected variant of Fragment 1 is His (Boc)-Aib-Glu(OtBu)-Gly-Thr(tBu)-Phe-Thr(tBu)-Ser(tBu)-Asp (OMpe)-Tyr(tBu)-Ser(tBu)-Ser(Psi(Me,Me)Pro)-OH (SEQ ID NO: 19) or a salt (e.g., a carboxylate salt) thereof.

In some embodiments, the protected variant of Fragment 1 is His (Boc)-Aib-Glu(OtBu)-Gly-Thr(tBu)-Phe-Thr(tBu)-Ser(tBu)-Asp (OMpe)-Tyr(tBu)-Ser(tBu)-Ser(Psi(Me,Me)Pro)-OH, wherein the protected variant of Fragment 1 has a protected N-terminus (SEQ ID NO: 24), or a salt (e.g., a carboxylate salt) thereof. In some embodiments, the protected variant of Fragment 1 is Boc-His (Boc)-Aib-Glu(OtBu)-Gly-Thr(tBu)-Phe-Thr(tBu)-Ser(tBu)-Asp (OMpe)-Tyr(tBu)-Ser(tBu)-Ser(Psi(Me,Me)Pro)-OH (SEQ ID NO: 93) or a salt (e.g., a carboxylate salt) thereof. In some embodiments, the protected variant of Fragment 1 is Fmoc-His (Boc)-Aib-Glu(OtBu)-Gly-Thr(tBu)-Phe-Thr(tBu)-Ser(tBu)-Asp (OMpe)-Tyr(tBu)-Ser(tBu)-Ser(Psi(Me,Me)Pro)-OH (SEQ ID NO: 94) or a salt (e.g., a carboxylate salt) thereof. In some embodiments, the protected variant of Fragment 1 is in the form of a carboxylate salt (e.g., a sodium or triethylammonium salt).

In some embodiments, the protected variant of Fragment 1 is His(Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 183) or a salt (e.g., a carboxylate salt) thereof.

In some embodiments, the protected variant of Fragment 1 is His(Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH, wherein the protected variant of Fragment 1 has a protected N-terminus (SEQ ID NO: 184), or a salt (e.g., a carboxylate salt) thereof. In some embodiments, the protected variant of Fragment 1 is Boc-His(Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 166) or a salt (e.g., a carboxylate salt) thereof. In some embodiments, the protected variant of Fragment 1 is Fmoc-His(Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 167) or a salt (e.g., a carboxylate salt) thereof. In some embodiments, the protected variant of Fragment 1 is in the form of a carboxylate salt (e.g., a sodium or triethylammonium salt).

In some embodiments, the protected variant of Fragment 1 is His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 185) or a salt (e.g., a carboxylate salt) thereof.

In some embodiments, the protected variant of Fragment 1 is His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH, wherein the protected variant of Fragment 1 has a protected N-terminus (SEQ ID NO: 186), or a salt (e.g., a carboxylate salt) thereof. In some embodiments, the protected variant of Fragment 1 is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 181) or a salt (e.g., a carboxylate salt) thereof. In some embodiments, the protected variant of Fragment 1 is Fmoc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 182) or a salt (e.g., a carboxylate salt) thereof. In some embodiments, the protected variant of Fragment 1 is in the form of a carboxylate salt (e.g., a sodium or triethylammonium salt).

Certain aspects provide a protected variant of Fragment 1, having a structure of

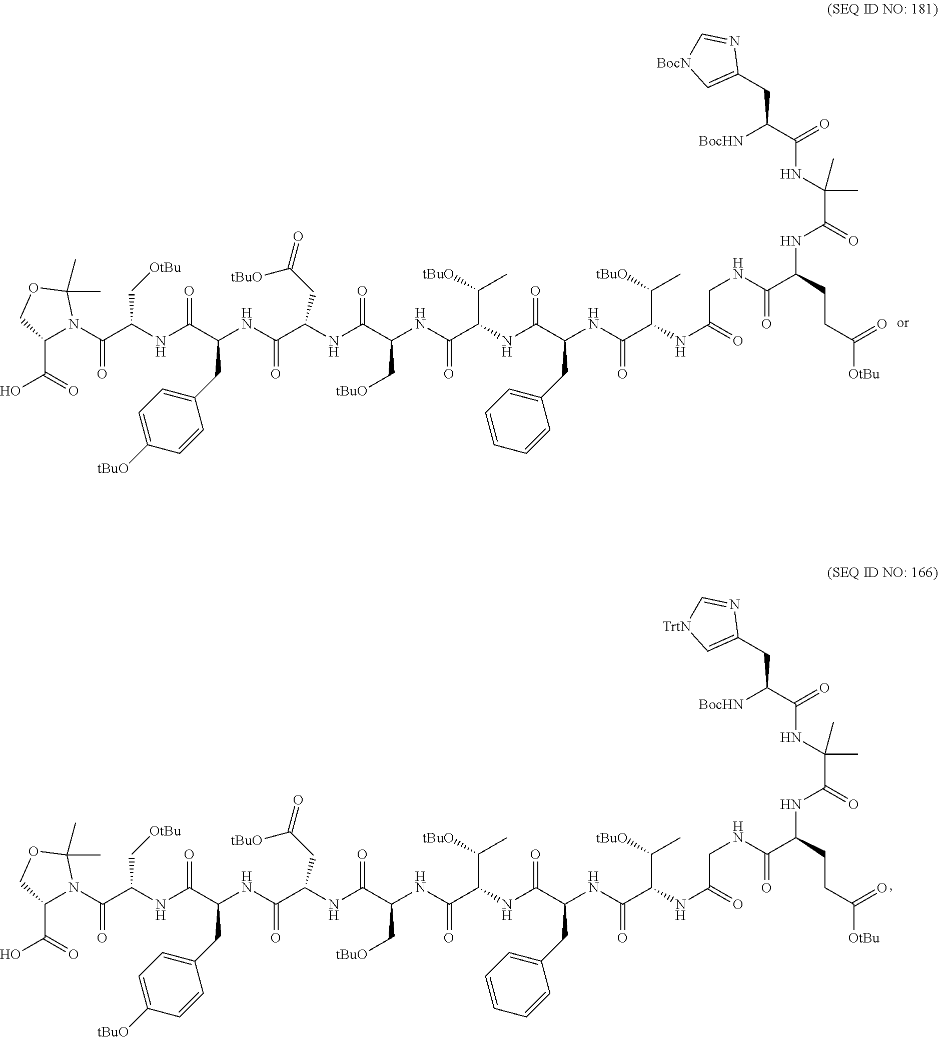

or a salt (e.g., a carboxylate salt) of either of the foregoing.

In some embodiments, the protected variant of Fragment 1 has the structure:

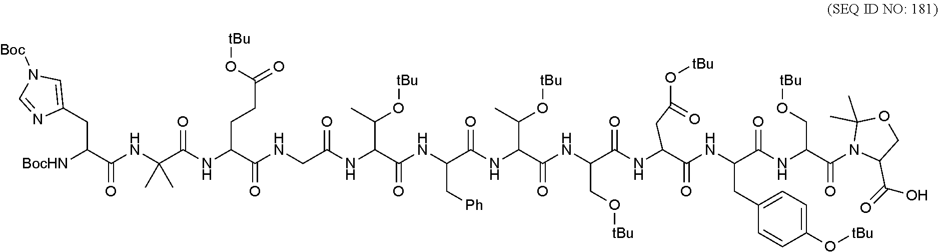 [SMILES: CC(OC(C)(C)C)C(NC(=O)CNC(=O)C(CCC(=O)OC(C)(C)C)NC(=O)C(C)(C)NC(=O)C(Cc1cn(C(=O)OC(C)(C)C)cn1)NC(=O)OC(C)(C)C)C(=O)NC(Cc1ccccc1)C(=O)NC(C(=O)NC(COC(C)(C)C)C(=O)NC(CC(=O)OC(C)(C)C)C(=O)NC(Cc1ccc(OC(C)(C)C)cc1)C(=O)NC(COC(C)(C)C)C(=O)N1C(C(=O)O)COC1(C)C)C(C)OC(C)(C)C]

or a salt (e.g., a carboxylate salt) thereof.

In some embodiments, the protected variant of Fragment 1 has the structure:

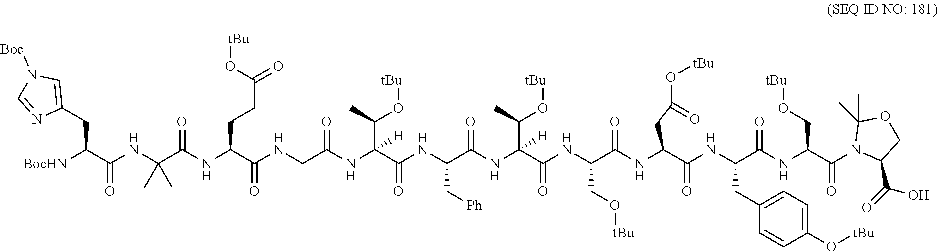 [SMILES: C[C@@H](OC(C)(C)C)[C@H](NC(=O)CNC(=O)[C@H](CCC(=O)OC(C)(C)C)NC(=O)C(C)(C)NC(=O)[C@H](Cc1cn(C(=O)OC(C)(C)C)cn1)NC(=O)OC(C)(C)C)C(=O)N[C@@H](Cc1ccccc1)C(=O)N[C@H](C(=O)N[C@@H](COC(C)(C)C)C(=O)N[C@@H](CC(=O)OC(C)(C)C)C(=O)N[C@@H](Cc1ccc(OC(C)(C)C)cc1)C(=O)N[C@@H](COC(C)(C)C)C(=O)N1[C@H](C(=O)O)COC1(C)C)[C@@H](C)OC(C)(C)C]

or a salt (e.g., a carboxylate salt) thereof.

In some embodiments, the protected variant of Fragment 1 has the structure:

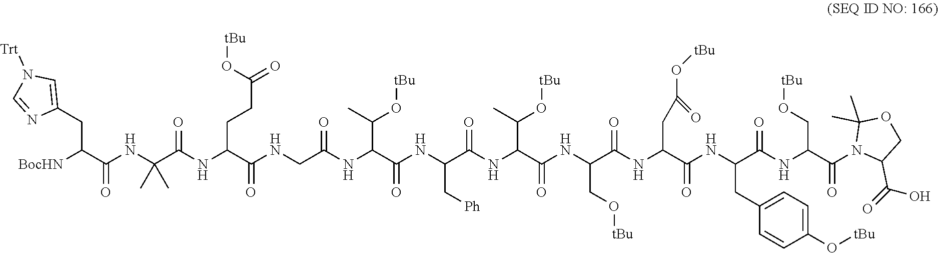

or a salt (e.g., a carboxylate salt) thereof.

In some embodiments, the protected variant of Fragment 1 has the structure:

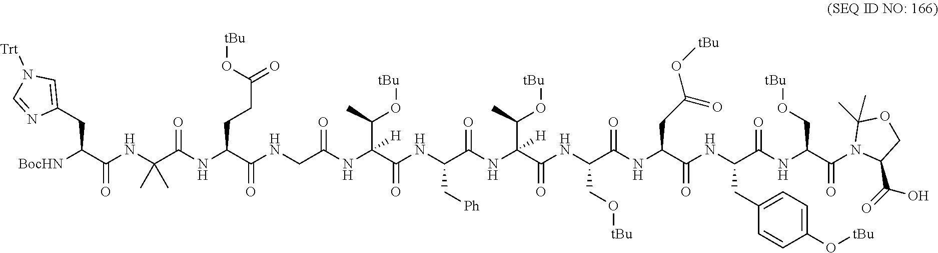

or a salt (e.g., a carboxylate salt) thereof.

### Fragment 1A

Also provided herein is a peptide having a sequence of His-Aib-Glu-Gly-Thr-Phe-Thr-Ser-Asp-Tyr-Ser-Ser-Tyr (SEQ ID NO: 11) (“Fragment 1A”). Certain aspects provide a protected variant of Fragment 1A, wherein at least one of the His, Glu, Thr, Ser, Asp, and Tyr residues is protected with a protecting group.

In some embodiments, the disclosure provides a protected variant of Fragment 1A, wherein:
the side chain of the His residue at position 1 is protected with a trityl (Trt), tert-butyloxycarbonyl (Boc), mesitylene-2-sulfonyl (Mts), monomethoxytrityl (Mmt), or 4-methyltrityl (Mtt) protecting group; and/or
the side chain of the Glu residue at position 3 is protected with a tert-butyl ester (OtBu), O-2-phenylisopropyl ester (OPp), or allyl ester (OAll) protecting group; and/or
the side chain of the Thr residue at position 5 or position 7 is protected with a tert-butyl (tBu), O-trityl (OTrt), O-allyl (OAll), or pseudoproline (Pro) protecting group; and/or
the side chain of the Ser residue at position 8, position 11, or position 12 is protected with a tert-butyl (tBu), O-trityl (OTrt), O-allyl (OAll), or pseudoproline (YPro) protecting group; and/or
the side chain of the Asp residue at position 9 is protected with a tert-butyl ester (OtBu), O-2-phenylisopropyl ester (OPp), or allyl ester (OAll) protecting group; and/or
the side chain of the Tyr residue at position 10 or position 13 is protected with a tert-butyl (tBu), O-allyl (OAll), trityl (Trt), or 2-chlorotrityl (Clt) protecting group.

In some embodiments, the N-terminus of the His residue at position 1 is protected with a 9-fluorenylmethoxycarbonyl (Fmoc) protecting group.

In some embodiments, the disclosure provides a protected variant of Fragment 1A, wherein:
the side chain of the His residue at position 1 is protected with a trityl (Trt) protecting group; and/or
the side chain of the Glu residue at position 3 is protected with a tert-butyl ester (OtBu) protecting group; and/or
the side chain of the Thr residue at position 5 or position 7 is protected with a tert-butyl (tBu) or O-trityl (OTrt) protecting group; and/or
the side chain of the Ser residue at position 8, position 11, or position 12 is protected with a tert-butyl (tBu) or O-trityl (OTrt) protecting group; and/or
the side chain of the Asp residue at position 9 is protected with a tert-butyl ester (OtBu) protecting group; and/or
the side chain of the Tyr residue at position 10 or position 13 is protected with a tert-butyl (tBu) protecting group.

In some embodiments, the N-terminus of the His residue at position 1 is protected with a 9-fluorenylmethoxycarbonyl (Fmoc) protecting group.

In some embodiments, the disclosure provides a protected variant of Fragment 1A, wherein:
the side chain of the His residue at position 1 is protected with a 2,4-dinitrophenyl (Dnp), p-toluenesulfonyl (Tos), o-nitrobenzyl (ONb), or benzyloxymethyl (Bom) protecting group; and/or
the side chain of the Glu residue at position 3 is protected with an allyl ester (OAll), benzyl ester (OBn), or cyclohexyl ester (OcHex) protecting group; and/or
the side chain of the Thr residue at position 5 or position 7 is protected with a p-bromobenzyloxycarbonyl (Br-Z), O-benzyl (OBn), or O-allyl (OAll) protecting group; and/or
the side chain of the Ser residue at position 8, position 11, or position 12 is protected with a p-bromobenzyloxycarbonyl (Br-Z), O-benzyl (OBn), or O-allyl (OAll) protecting group; and/or
the side chain of the Asp residue at position 9 is protected with an allyl ester (OAll), benzyl ester (OBn), or cyclohexyl ester (OcHex) protecting group; and/or
the side chain of the Tyr residue at position 10 or position 13 is protected with an O-benzyl (OBn), O-allyl (OAll), O-(2-bromobenzyl), or 2,6-dichlorobenzyl (Dcb) protecting group.

In some embodiments, the N-terminus of the His residue at position 1 is protected with a tert-butyloxycarbonyl (Boc) protecting group.

In some embodiments, the disclosure provides a protected variant of Fragment 1A, wherein:
the side chain of the His residue at position 1 is protected with a p-toluenesulfonyl (Tos) protecting group; and/or
the side chain of the Glu residue at position 3 is protected with a benzyl ester (OBn) or allyl ester (OAll) protecting group; and/or
the side chain of the Thr residue at position 5 or position 7 is protected with a O-benzyl (OBn) protecting group; and/or
the side chain of the Ser residue at position 8, position 11, or position 12 is protected with a O-benzyl (OBn) protecting group; and/or
the side chain of the Asp residue at position 9 is protected with a benzyl ester (OBn) or allyl ester (OAll) protecting group; and/or
the side chain of the Tyr residue at position 10 or position 13 is protected with a O-benzyl (OBn) protecting group.

In some embodiments, the N-terminus of the His residue at position 1 is protected with a tert-butyloxycarbonyl (Boc) protecting group.

Certain aspects provide a protected variant of Fragment 1A, wherein the N-terminal His residue is protected with a Boc, Fmoc, or Trt protecting group. In some embodiments, the His residue is protected with a Boc or Fmoc protecting group. Certain aspects provide a protected variant of Fragment 1A, wherein the N-terminal His is protected with a Boc protecting group. Certain aspects provide a protected variant of Fragment 1A, wherein the Glu residue at position 3 is protected as a t-butyl ester (OtBu). Certain aspects provide a protected variant of Fragment 1A, wherein the Thr residue at position 5 is protected as a t-butyl ether (tBu). Certain aspects provide a protected variant of Fragment 1A, wherein the Thr residue at position 7 is protected as a t-butyl ether (tBu). Certain aspects provide a protected variant of Fragment 1A, wherein the Ser residue at position 8 is protected as a t-butyl ether (tBu). Certain aspects provide a protected variant of Fragment 1A, wherein the Asp residue at position 9 is protected as a t-butyl ester (OtBu). Certain aspects provide a protected variant of Fragment 1A, wherein the Tyr residue at position 10 is protected as a t-butyl ether (tBu). Certain aspects provide a protected variant of Fragment 1A, wherein the Ser residue at position 11 is protected as a t-butyl ether (tBu). Certain aspects provide a protected variant of Fragment 1A, wherein the Ser residue at position 12 is protected as a t-butyl ether (tBu). Certain aspects provide a protected variant of Fragment 1A, wherein the Tyr at position 13 is protected as a t-butyl ether (tBu).

In some embodiments, the present disclosure provides a protected variant of Fragment 1A, wherein:
the Glu residue at position 3 is protected as a t-butyl ester (OtBu); and/or
the Thr residue at position 5 is protected as a t-butyl ether (tBu); and/or
the Thr residue at position 7 is protected as a t-butyl ether (tBu); and/or
the Ser residue at position 8 is protected as a t-butyl ether (tBu); and/or
the Asp residue at position 9 is protected as a t-butyl ester (OtBu); and/or
the Tyr residue at position 10 is protected as a t-butyl ether (tBu); and/or
the Ser residue at position 11 is protected as a t-butyl ether (tBu); and/or
the Ser residue at position 12 is protected as a t-butyl ether (tBu); and/or
the Tyr at position 13 is protected as a t-butyl ether (tBu).

In some embodiments, the His residue at position 1 is protected with a Boc or Trt protecting group. In some embodiments, the His residue at position 1 is protected with a Boc protecting group. In some embodiments, the His residue at position 1 is protected with a Trt protecting group. In some embodiments, the N-terminus of the His residue at position 1 is protected with a Boc or Fmoc protecting group. In some embodiments, the N-terminus of the His residue at position 1 is protected with a Boc protecting group. In some embodiments, the N-terminus of the His residue at position 1 is protected with a Fmoc protecting group.

In some embodiments, the present disclosure provides a protected variant of Fragment 1A, wherein:
the Glu residue at position 3 is protected as a t-butyl ester (OtBu);
the Thr residue at position 5 is protected as a t-butyl ether (tBu);
the Thr residue at position 7 is protected as a t-butyl ether (tBu);
the Ser residue at position 8 is protected as a t-butyl ether (tBu);
the Asp residue at position 9 is protected as a t-butyl ester (OtBu);
the Tyr residue at position 10 is protected as a t-butyl ether (tBu);
the Ser residue at position 11 is protected as a t-butyl ether (tBu);
the Ser residue at position 12 is protected as a t-butyl ether (tBu); and
the Tyr at position 13 is protected as a t-butyl ether (tBu).

In some embodiments, the His residue at position 1 is protected with a Boc or Trt protecting group. In some embodiments, the His residue at position 1 is protected with a Boc protecting group. In some embodiments, the His residue at position 1 is protected with a Trt protecting group. In some embodiments, the N-terminus of the His residue at position 1 is protected with a Boc or Fmoc protecting group. In some embodiments, the N-terminus of the His residue at position 1 is protected with a Boc protecting group. In some embodiments, the N-terminus of the His residue at position 1 is protected with a Fmoc protecting group.

In some embodiments, the protected variant of Fragment 1A is His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH (SEQ ID NO: 20) or a salt (e.g., a carboxylate salt) thereof.

In some embodiments, the protected variant of Fragment 1A is His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH, wherein the protected variant of Fragment 1A has a protected N-terminus (SEQ ID NO: 25), or a salt (e.g., a carboxylate salt) thereof. In some embodiments, the protected variant of Fragment 1A is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH (SEQ ID NO: 95) or a salt (e.g., a carboxylate salt) thereof. In some embodiments, the protected variant of Fragment 1A is Fmoc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH (SEQ ID NO: 99) or a salt (e.g., a carboxylate salt) thereof.

In some embodiments, the protected variant of Fragment 1A is His(Trt)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH (SEQ ID NO: 21) or a salt (e.g., a carboxylate salt) thereof.

In some embodiments, the protected variant of Fragment 1A is His(Trt)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH, wherein the protected variant of Fragment 1A has a protected N-terminus (SEQ ID NO: 26), or a salt (e.g., a carboxylate salt) thereof. In some embodiments, the protected variant of Fragment 1A is Boc-His(Trt)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH (SEQ ID NO: 100) or a salt (e.g., a carboxylate salt) thereof. In some embodiments, the protected variant of Fragment 1A is Fmoc-His(Trt)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH (SEQ ID NO: 101) or a salt (e.g., a carboxylate salt) thereof.

Certain aspects provide a protected variant of Fragment 1A, having a structure of:

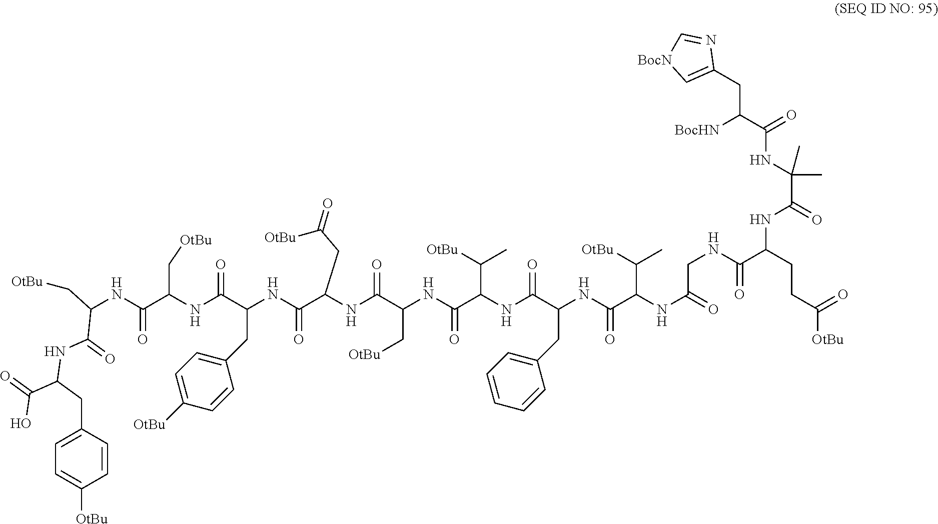 [SMILES: CC(OC(C)(C)C)C(NC(=O)CNC(=O)C(CCC(=O)OC(C)(C)C)NC(=O)C(C)(C)NC(=O)C(Cc1cn(C(=O)OC(C)(C)C)cn1)NC(=O)OC(C)(C)C)C(=O)NC(Cc1ccccc1)C(=O)NC(C(=O)NC(COC(C)(C)C)C(=O)NC(CC(=O)OC(C)(C)C)C(=O)NC(Cc1ccc(OC(C)(C)C)cc1)C(=O)NC(COC(C)(C)C)C(=O)NC(COC(C)(C)C)C(=O)NC(Cc1ccc(OC(C)(C)C)cc1)C(=O)O)C(C)OC(C)(C)C]

or a salt (e.g., a carboxylate salt) thereof.

In some embodiments, the protected variant of Fragment 1A has the structure:

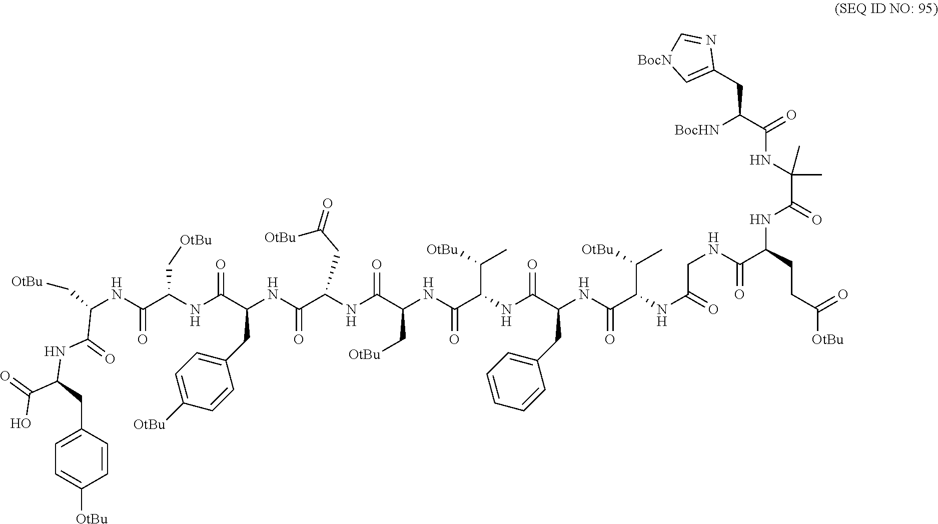 [SMILES: C[C@@H](OC(C)(C)C)[C@H](NC(=O)CNC(=O)[C@H](CCC(=O)OC(C)(C)C)NC(=O)C(C)(C)NC(=O)[C@H](Cc1cn(C(=O)OC(C)(C)C)cn1)NC(=O)OC(C)(C)C)C(=O)N[C@@H](Cc1ccccc1)C(=O)N[C@H](C(=O)N[C@@H](COC(C)(C)C)C(=O)N[C@@H](CC(=O)OC(C)(C)C)C(=O)N[C@@H](Cc1ccc(OC(C)(C)C)cc1)C(=O)N[C@@H](COC(C)(C)C)C(=O)N[C@@H](COC(C)(C)C)C(=O)N[C@@H](Cc1ccc(OC(C)(C)C)cc1)C(=O)O)[C@@H](C)OC(C)(C)C]

or a salt (e.g., a carboxylate salt) thereof.

### Fragment 2

Also provided herein is a peptide having a sequence of Tyr-Leu-Glu-Glu-Gln-Ala-Ala-Lys-Glu-Phe-Ile-Ala (SEQ ID NO: 2) (“Fragment 2”). Certain aspects provide a protected variant of Fragment 2, wherein at least one of the Tyr, Glu, Gln, and Lys residues is protected with a protecting group.

In some embodiments, the disclosure provides a protected variant of Fragment 2, wherein:
the side chain of the Tyr residue at position 1 is protected with a tert-butyl (tBu), O-allyl (OAll), trityl (Trt), or 2-chlorotrityl (Clt) protecting group; and/or
the side chain of the Glu residue at position 3, position 4, or position 9 is protected with a tert-butyl ester (OtBu), O-2-phenylisopropyl ester (OPp), or allyl ester (OAll) protecting group; and/or
the side chain of the Gln residue at position 5 is protected with a trityl (Trt), benzyloxymethyl (Bom), xanthenyl (Xan), or p-methoxybenzyl (PMB) protecting group; and/or
the side chain of the Lys residue at position 8 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), 4-methyltrityl (Mtt), or benzyloxycarbonyl (Cbz) protecting group.

In some embodiments, the N-terminus of the Tyr residue at position 1 is protected with a 9-fluorenylmethoxycarbonyl (Fmoc) protecting group.

In some embodiments, the disclosure provides a protected variant of Fragment 2, wherein:
the side chain of the Tyr residue at position 1 is protected with a tert-butyl (tBu) protecting group; and/or
the side chain of the Glu residue at position 3, position 4, or position 9 is protected with a tert-butyl ester (OtBu) protecting group; and/or
the side chain of the Gln residue at position 5 is protected with a trityl (Trt) protecting group; and/or
the side chain of the Lys residue at position 8 is protected with a tert-butyloxycarbonyl (Boc), 1-(4,4-dimethyl-2,6-dioxocyclohex-1-ylidene)ethyl (Dde), or allyloxycarbonyl (Alloc) protecting group.

In some embodiments, the N-terminus of the Tyr residue at position 1 is protected with a 9-fluorenylmethoxycarbonyl (Fmoc) protecting group.

In some embodiments, the disclosure provides a protected variant of Fragment 2, wherein:
the side chain of the Tyr residue at position 1 is protected with an O-benzyl (OBn), O-allyl (OAll), O-(2-bromobenzyl), or 2,6-dichlorobenzyl (Dcb) protecting group; and/or
the side chain of the Glu residue at position 3, position 4, or position 9 is protected with an allyl ester (OAll), benzyl ester (OBn), or cyclohexyl ester (OcHex) protecting group; and/or
the side chain of the Lys residue at position 8 is protected with a benzyloxycarbonyl (Cbz), 2-chlorobenzyloxycarbonyl (2-Cl—Z), or allyloxycarbonyl (Alloc) protecting group.

In some embodiments, the N-terminus of the Tyr residue at position 1 is protected with a tert-butyloxycarbonyl (Boc) protecting group.

In some embodiments, the disclosure provides a protected variant of Fragment 2, wherein:
the side chain of the Tyr residue at position 1 is protected with an O-benzyl (OBn) protecting group; and/or
the side chain of the Glu residue at position 3, position 4, or position 9 is protected with a benzyl ester (OBn) or allyl ester (OAll) protecting group; and/or
the side chain of the Lys residue at position 8 is protected with a benzyloxycarbonyl (Cbz) or 2-chlorobenzyloxycarbonyl(2-Cl—Z) protecting group.

In some embodiments, the N-terminus of the Tyr residue at position 1 is protected with a tert-butyloxycarbonyl (Boc) protecting group.

Certain aspects disclose a protected variant of Fragment 2, wherein the Tyr residue at position 1 is protected as a t-butyl ether (tBu). Certain aspects provide a protected variant of Fragment 2, wherein the Tyr at position 1 is protected with an Fmoc protecting group at the N-terminus. Certain aspects provide a protected variant of Fragment 2, wherein the Glu residue at position 3 is protected as a t-butyl ester (OtBu). Certain aspects provide a protected variant of Fragment 2, wherein the Glu residue at position 4 is protected as a t-butyl ester (OtBu). Certain aspects provide a protected variant of Fragment 2, wherein the Gln residue at position 5 is protected as a triphenylmethyl amide (Trt). Certain aspects provide a protected variant of Fragment 2, wherein the Lys residue at position 8 is protected as a t-butyloxy carbonyl amide (Boc). Certain aspects provide a protected variant of Fragment 2, wherein the Glu residue at position 9 is protected as a t-butyl ester (OtBu).

In some embodiments, the present disclosure provides a protected variant of Fragment 2, wherein:
the Tyr residue at position 1 is protected as a t-butyl ether (tBu) and/or the Tyr residue at position 1 is protected with an Fmoc protecting group at the N-terminus; and/or
the Glu residue at position 3 is protected as a t-butyl ester (OtBu); and/or
the Glu residue at position 4 is protected as a t-butyl ester (OtBu); and/or
the Gln residue at position 5 is protected as a triphenylmethyl amide (Trt); and/or
the Lys residue at position 8 is protected as a t-butyloxy carbonyl amide (Boc); and/or
the Glu residue at position 9 is protected as a t-butyl ester (OtBu).

In some embodiments, the Tyr residue at position 1 is protected as a t-butyl ether (tBu). In some embodiments, the Tyr residue at position 1 is protected with an Fmoc protecting group at the N-terminus. In some embodiments, the Tyr residue at position 1 is protected as a t-butyl ether (tBu), and the Tyr residue at position 1 is protected with an Fmoc protecting group at the N-terminus.

In some embodiments, the present disclosure provides a protected variant of Fragment 2, wherein:
the Tyr residue at position 1 is protected as a t-butyl ether (tBu) and/or the Tyr residue at position 1 is protected with an Fmoc protecting group at the N-terminus;
the Glu residue at position 3 is protected as a t-butyl ester (OtBu);
the Glu residue at position 4 is protected as a t-butyl ester (OtBu);
the Gln residue at position 5 is protected as a triphenylmethyl amide (Trt);
the Lys residue at position 8 is protected as a t-butyloxy carbonyl amide (Boc); and
the Glu residue at position 9 is protected as a t-butyl ester (OtBu).

In some embodiments, the Tyr residue at position 1 is protected as a t-butyl ether (tBu). In some embodiments, the Tyr residue at position 1 is protected with an Fmoc protecting group at the N-terminus. In some embodiments, the Tyr residue at position 1 is protected as a t-butyl ether (tBu), and the Tyr residue at position 1 is protected with an Fmoc protecting group at the N-terminus.

In some embodiments, the protected variant of Fragment 2 is Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 28) or a salt thereof. In some embodiments, the protected variant of Fragment 2 is in the form of an acid-addition salt (e.g., a TFA, HCl, or acetate salt). In some embodiments, the protected variant of Fragment 2 is in the form of a carboxylate salt.

In some embodiments, the protected variant of Fragment 2 is Tyr(tBu)-Leu-Glu(OtBu)-Glu(OtBu)-Gln(Trt)-Ala-Ala-Lys(Boc)-Glu(OtBu)-Phe-Ile-Ala-OH, wherein the protected variant of Fragment 2 has a protected N-terminus (SEQ ID NO: 36), or a salt (e.g., a carboxylate salt) thereof. In some embodiments, the protected variant of Fragment 2 is Fmoc-Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 52) or a salt (e.g., a carboxylate salt) thereof.

In some embodiments, the protected variant of Fragment 2 is Tyr(tBu)-Leu-Glu(OtBu)-Glu(OtBu)-Gln(Trt)-Ala-Ala-Lys(Boc)-Glu(OtBu)-Phe-Ile-Ala, wherein the protected variant of Fragment 2 has a protected N-terminus (e.g., an Fmoc-protected N-terminus) and a protected C-terminus (SEQ ID NO: 102).

In some embodiments, the protected variant of Fragment 2 is Tyr(tBu)-Leu-Glu(OtBu)-Glu(OtBu)-Gln(Trt)-Ala-Ala-Lys(Boc)-Glu(OtBu)-Phe-Ile-Ala, wherein the protected variant of Fragment 2 has a protected C-terminus (SEQ ID NO: 44), or a salt (e.g., an acid-addition salt) thereof.

Certain aspects provide a protected variant of Fragment 2, having a structure of:

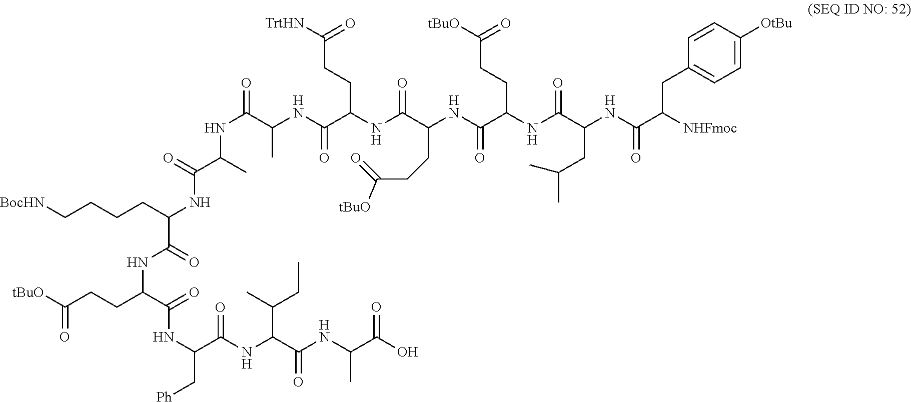

or a salt (e.g., a carboxylate salt) thereof.

In some embodiments, the protected variant of Fragment 2 has the structure:

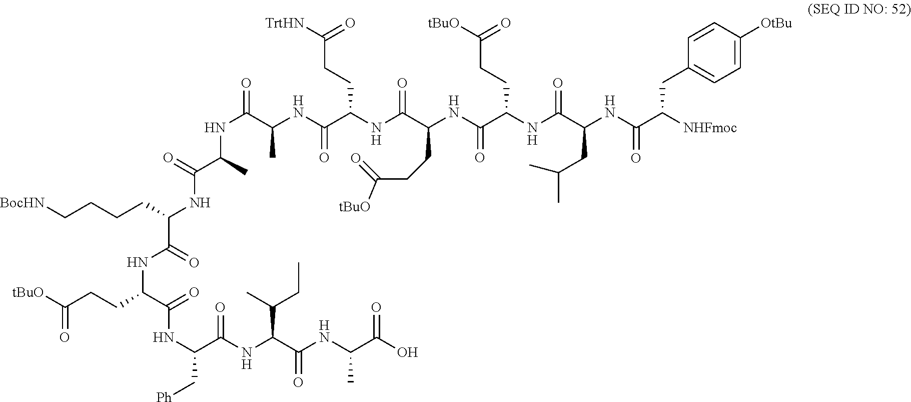

or a salt (e.g., a carboxylate salt) thereof.

### Fragment 2A

Also provided herein is a peptide having a sequence of Leu-Glu-Glu-Gln-Ala-Ala-Lys-Glu-Phe-Ile (SEQ ID NO: 12) (“Fragment 2A”). Certain aspects provide a protected variant of Fragment 2A, wherein at least one of the Glu, Gln, and Lys residues is protected with a protecting group.

In some embodiments, the disclosure provides a protected variant of Fragment 2A, wherein:
the side chain of the Glu residue at position 2, position 3, or position 8 is protected with a tert-butyl ester (OtBu), O-2-phenylisopropyl ester (OPp), or allyl ester (OAll) protecting group; and/or
the side chain of the Gln residue at position 4 is protected with a trityl (Trt), benzyloxymethyl (Bom), xanthenyl (Xan), or p-methoxybenzyl (PMB) protecting group; and/or
the side chain of the Lys residue at position 7 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), 4-methyltrityl (Mtt), or benzyloxycarbonyl (Cbz) protecting group.

In some embodiments, the N-terminus of the Leu residue at position 1 is protected with a 9-fluorenylmethoxycarbonyl (Fmoc) protecting group.

In some embodiments, the disclosure provides a protected variant of Fragment 2A, wherein:
the side chain of the Glu residue at position 2, position 3, or position 8 is protected with a tert-butyl ester (OtBu) protecting group; and/or
the side chain of the Gln residue at position 4 is protected with a trityl (Trt) protecting group; and/or
the side chain of the Lys residue at position 7 is protected with a tert-butyloxycarbonyl (Boc), 1-(4,4-dimethyl-2,6-dioxocyclohex-1-ylidene)ethyl (Dde), or allyloxycarbonyl (Alloc) protecting group.

In some embodiments, the N-terminus of the Leu residue at position 1 is protected with a 9-fluorenylmethoxycarbonyl (Fmoc) protecting group.

In some embodiments, the disclosure provides a protected variant of Fragment 2A, wherein:
the side chain of the Glu residue at position 2, position 3, or position 8 is protected with an allyl ester (OAll), benzyl ester (OBn), or cyclohexyl ester (OcHex) protecting group; and/or
the side chain of the Lys residue at position 7 is protected with a benzyloxycarbonyl (Cbz), 2-chlorobenzyloxycarbonyl(2-Cl—Z), or allyloxycarbonyl (Alloc) protecting group.

In some embodiments, the N-terminus of the Leu residue at position 1 is protected with a tert-butyloxycarbonyl (Boc) protecting group.

In some embodiments, the disclosure provides a protected variant of Fragment 2A, wherein:
the side chain of the Glu residue at position 2, position 3, or position 8 is protected with a benzyl ester (OBn) or allyl ester (OAll) protecting group; and/or
the side chain of the Lys residue at position 7 is protected with a benzyloxycarbonyl (Cbz) or 2-chlorobenzyloxycarbonyl(2-Cl—Z) protecting group.

In some embodiments, the N-terminus of the Leu residue at position 1 is protected with a tert-butyloxycarbonyl (Boc) protecting group.

Certain aspects provide a protected variant of Fragment 2A, wherein the Glu residue at position 2 is protected as a t-butyl ester (OtBu). Certain aspects provide a protected variant of Fragment 2A, wherein the Glu residue at position 3 is protected as a t-butyl ester (OtBu). Certain aspects provide a protected variant of Fragment 2A, wherein the Gln residue at position 4 is protected as a triphenylmethyl amide (Trt). Certain aspects provide a protected variant of Fragment 2A, wherein the Lys residue at position 7 is protected as a t-butyloxy carbonyl amide (Boc). Certain aspects provide a protected variant of Fragment 2A, wherein the Glu residue at position 8 is protected as a t-butyloxy carbonyl amide (Boc).

In some embodiments, the present disclosure provides a protected variant of Fragment 2A, wherein:
the Glu residue at position 2 is protected as a t-butyl ester (OtBu); and/or
the Glu residue at position 3 is protected as a t-butyl ester (OtBu); and/or
the Gln residue at position 4 is protected as a triphenylmethyl amide (Trt); and/or
the Lys residue at position 7 is protected as a t-butyloxy carbonyl amide (Boc); and/or
the Glu residue at position 8 is protected as a t-butyl ester (OtBu).

In some embodiments, the Leu residue at position 1 is protected with an Fmoc protecting group at the N-terminus.

In some embodiments, the present disclosure provides a protected variant of Fragment 2A, wherein:
the Glu residue at position 2 is protected as a t-butyl ester (OtBu);
the Glu residue at position 3 is protected as a t-butyl ester (OtBu);
the Gln residue at position 4 is protected as a triphenylmethyl amide (Trt);
the Lys residue at position 7 is protected as a t-butyloxy carbonyl amide (Boc); and
the Glu residue at position 8 is protected as a t-butyl ester (OtBu).

In some embodiments, the Leu residue at position 1 is protected with an Fmoc protecting group at the N-terminus.

In some embodiments, the protected variant of Fragment 2A is Leu-Glu(OtBu)-Glu(OtBu)-Gln(Trt)-Ala-Ala-Lys(Boc)-Glu(OtBu)-Phe-Ile-OH (SEQ ID NO: 29) or a salt thereof. In some embodiments, the protected variant of Fragment 2A is in the form of an acid-addition salt. In some embodiments, the protected variant of Fragment 2A is in the form of a carboxylate salt.

In some embodiments, the protected variant of Fragment 2A is Leu-Glu(OtBu)-Glu(OtBu)-Gln(Trt)-Ala-Ala-Lys(Boc)-Glu(OtBu)-Phe-Ile-OH, wherein the protected variant of Fragment 2A has a protected N-terminus (SEQ ID NO: 37), or a salt (e.g., a carboxylate salt) thereof. In some embodiments, the protected variant of Fragment 2A is Fmoc-Leu-Glu(OtBu)-Glu(OtBu)-Gln(Trt)-Ala-Ala-Lys(Boc)-Glu(OtBu)-Phe-Ile-OH (SEQ ID NO: 53) or a salt (e.g., a carboxylate salt) thereof.

In some embodiments, the protected variant of Fragment 2A is Leu-Glu(OtBu)-Glu(OtBu)-Gln(Trt)-Ala-Ala-Lys(Boc)-Glu(OtBu)-Phe-Ile, wherein the protected variant of Fragment 2A has a protected N-terminus (e.g., an Fmoc-protected N-terminus) and a protected C-terminus (SEQ ID NO: 103).

In some embodiments, the protected variant of Fragment 2A is Leu-Glu(OtBu)-Glu(OtBu)-Gln(Trt)-Ala-Ala-Lys(Boc)-Glu(OtBu)-Phe-Ile, wherein the protected variant of Fragment 2A has a protected C-terminus (SEQ ID NO: 45), or a salt (e.g., an acid-addition salt) thereof.

Certain aspects provide a protected variant of Fragment 2A, having a structure of

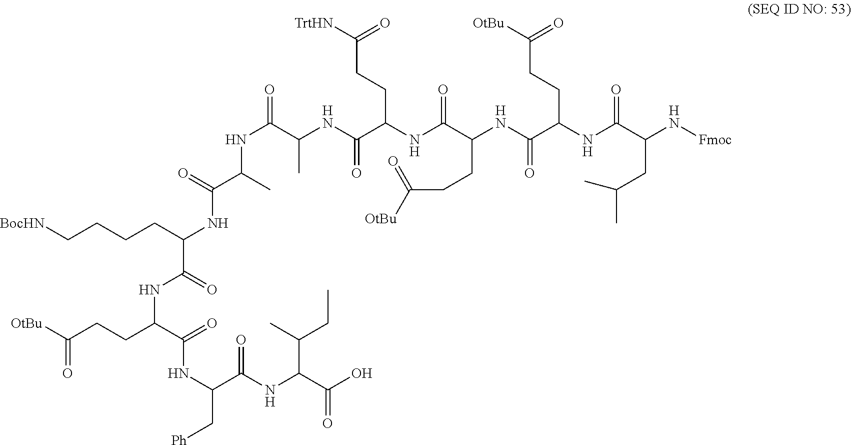

or a salt (e.g., a carboxylate salt) thereof.

In some embodiments, the protected variant of Fragment 2A has the structure:

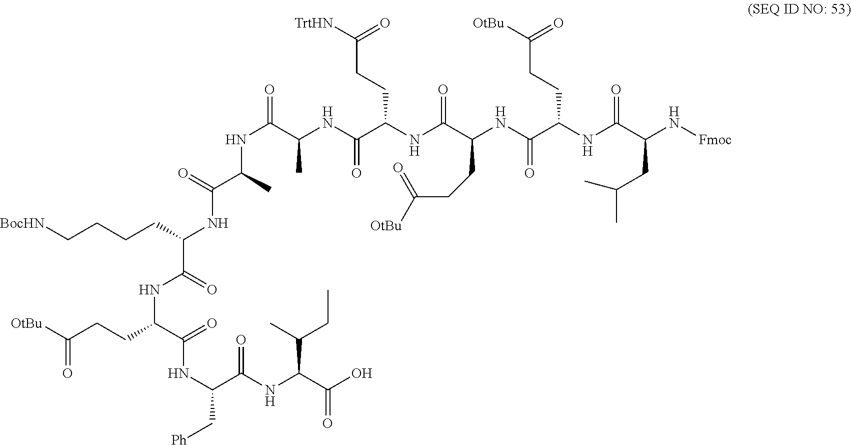

or a salt (e.g., a carboxylate salt) thereof.

### Fragment 2B

Also provided herein is a peptide having a sequence of Tyr-Leu-Glu-Glu-Gln-Ala-Ala-Lys-Glu-Phe-Ile (SEQ ID NO: 16) (“Fragment 2B”). Certain aspects provide a protected variant of Fragment 2B, wherein at least one of the Tyr, Glu, Gln, and Lys residues is protected with a protecting group.

In some embodiments, the disclosure provides a protected variant of Fragment 2B, wherein:
the side chain of the Tyr residue at position 1 is protected with a tert-butyl (tBu), O-allyl (OAll), trityl (Trt), or 2-chlorotrityl (Clt) protecting group; and/or
the side chain of the Glu residue at position 3, position 4, or position 9 is protected with a tert-butyl ester (OtBu), O-2-phenylisopropyl ester (OPp), or allyl ester (OAll) protecting group; and/or
the side chain of the Gln residue at position 5 is protected with a trityl (Trt), benzyloxymethyl (Bom), xanthenyl (Xan), or p-methoxybenzyl (PMB) protecting group; and/or
the side chain of the Lys residue at position 8 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), 4-methyltrityl (Mtt), or benzyloxycarbonyl (Cbz) protecting group.

In some embodiments, the N-terminus of the Tyr residue at position 1 is protected with a 9-fluorenylmethoxycarbonyl (Fmoc) protecting group.

In some embodiments, the disclosure provides a protected variant of Fragment 2B, wherein:
the side chain of the Tyr residue at position 1 is protected with a tert-butyl (tBu) protecting group; and/or
the side chain of the Glu residue at position 3, position 4, or position 9 is protected with a tert-butyl ester (OtBu) protecting group; and/or
the side chain of the Gln residue at position 5 is protected with a trityl (Trt) protecting group; and/or
the side chain of the Lys residue at position 8 is protected with a tert-butyloxycarbonyl (Boc), 1-(4,4-dimethyl-2,6-dioxocyclohex-1-ylidene)ethyl (Dde), or allyloxycarbonyl (Alloc) protecting group.

In some embodiments, the N-terminus of the Tyr residue at position 1 is protected with a 9-fluorenylmethoxycarbonyl (Fmoc) protecting group.

In some embodiments, the disclosure provides a protected variant of Fragment 2B, wherein:
the side chain of the Tyr residue at position 1 is protected with an O-benzyl (OBn), O-allyl (OAll), O-(2-bromobenzyl), or 2,6-dichlorobenzyl (Dcb) protecting group; and/or
the side chain of the Glu residue at position 3, position 4, or position 9 is protected with an allyl ester (OAll), benzyl ester (OBn), or cyclohexyl ester (OcHex) protecting group; and/or
the side chain of the Lys residue at position 8 is protected with a benzyloxycarbonyl (Cbz), 2-chlorobenzyloxycarbonyl (2-Cl—Z), or allyloxycarbonyl (Alloc) protecting group.

In some embodiments, the N-terminus of the Tyr residue at position 1 is protected with a tert-butyloxycarbonyl (Boc) protecting group.

In some embodiments, the disclosure provides a protected variant of Fragment 2B, wherein:
the side chain of the Tyr residue at position 1 is protected with an O-benzyl (OBn) protecting group; and/or
the side chain of the Glu residue at position 3, position 4, or position 9 is protected with a benzyl ester (OBn) or allyl ester (OAll) protecting group; and/or
the side chain of the Lys residue at position 8 is protected with a benzyloxycarbonyl (Cbz) or 2-chlorobenzyloxycarbonyl(2-Cl—Z) protecting group.

In some embodiments, the N-terminus of the Tyr residue at position 1 is protected with a tert-butyloxycarbonyl (Boc) protecting group.

Certain aspects provide a protected variant of Fragment 2B, wherein at least one of the Glu, Gln, and Lys residues is protected with a protecting group. Certain aspects provide a protected variant of Fragment 2B, wherein the Tyr residue at position 1 is protected as a t-butyl ether (tBu). Certain aspects provide a protected variant of Fragment 2B, wherein the Glu residue at position 3 is protected as a t-butyl ester (OtBu). Certain aspects provide a protected variant of Fragment 2B, wherein the Glu residue at position 4 is protected as a t-butyl ester (OtBu). Certain aspects provide a protected variant of Fragment 2B, wherein the Gln residue at position 5 is protected as a triphenylmethyl amide (Trt). Certain aspects provide a protected variant of Fragment 2B, wherein the Lys residue at position 8 is protected as a t-butyloxy carbonyl amide (Boc). Certain aspects provide a protected variant of Fragment 2B, wherein the Glu residue at position 9 is protected as a t-butyl ester (OtBu).

In some embodiments, the present disclosure provides a protected variant of Fragment 2B, wherein:
the Tyr residue at position 1 is protected as a t-butyl ether (tBu); and/or
the Glu residue at position 3 is protected as a t-butyl ester (OtBu); and/or
the Glu residue at position 4 is protected as a t-butyl ester (OtBu); and/or
the Gln residue at position 5 is protected as a triphenylmethyl amide (Trt); and/or
the Lys residue at position 8 is protected as a t-butyloxy carbonyl amide (Boc); and/or
the Glu residue at position 9 is protected as a t-butyl ester (OtBu).

In some embodiments, the present disclosure provides a protected variant of Fragment 2B, wherein:
the Tyr residue at position 1 is protected as a t-butyl ether (tBu);
the Glu residue at position 3 is protected as a t-butyl ester (OtBu);
the Glu residue at position 4 is protected as a t-butyl ester (OtBu);
the Gln residue at position 5 is protected as a triphenylmethyl amide (Trt);
the Lys residue at position 8 is protected as a t-butyloxy carbonyl amide (Boc); and
the Glu residue at position 9 is protected as a t-butyl ester (OtBu).

In some embodiments, the protected variant of Fragment 2B is Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 30) or a salt thereof. In some embodiments, the protected variant of Fragment 2B is in the form of an acid-addition salt. In some embodiments, the protected variant of Fragment 2B is in the form of a carboxylate salt.

In some embodiments, the protected variant of Fragment 2B is Tyr(tBu)-Leu-Glu(OtBu)-Glu(OtBu)-Gln(Trt)-Ala-Ala-Lys(Boc)-Glu(OtBu)-Phe-Ile-OH, wherein the protected variant of Fragment 2B has a protected N-terminus (SEQ ID NO: 38), or a salt (e.g., a carboxylate salt) thereof. In some embodiments, the protected variant of Fragment 2B is Fmoc-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 54) or a salt (e.g., a carboxylate salt) thereof.

In some embodiments, the protected variant of Fragment 2B is Tyr(tBu)-Leu-Glu(OtBu)-Glu(OtBu)-Gln(Trt)-Ala-Ala-Lys(Boc)-Glu(OtBu)-Phe-Ile, wherein the protected variant of Fragment 2B has a protected N-terminus (e.g., an Fmoc-protected N-terminus) and a protected C-terminus (SEQ ID NO: 104).

In some embodiments, the protected variant of Fragment 2B is Tyr(tBu)-Leu-Glu(OtBu)-Glu(OtBu)-Gln(Trt)-Ala-Ala-Lys(Boc)-Glu(OtBu)-Phe-Ile, wherein the protected variant of Fragment 2B has a protected C-terminus (SEQ ID NO: 46), or a salt (e.g., an acid-addition salt) thereof.

Certain aspects provide a protected variant of Fragment 2B, having a structure of

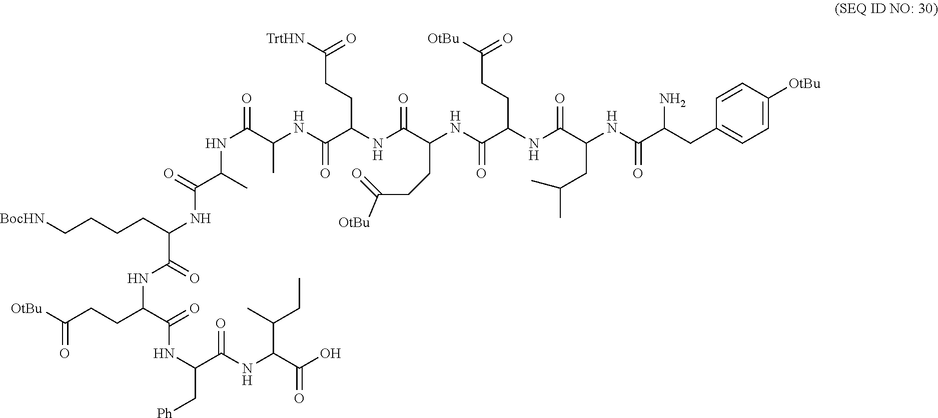

or a salt thereof.

In some embodiments, the protected variant of Fragment 2B has the structure:

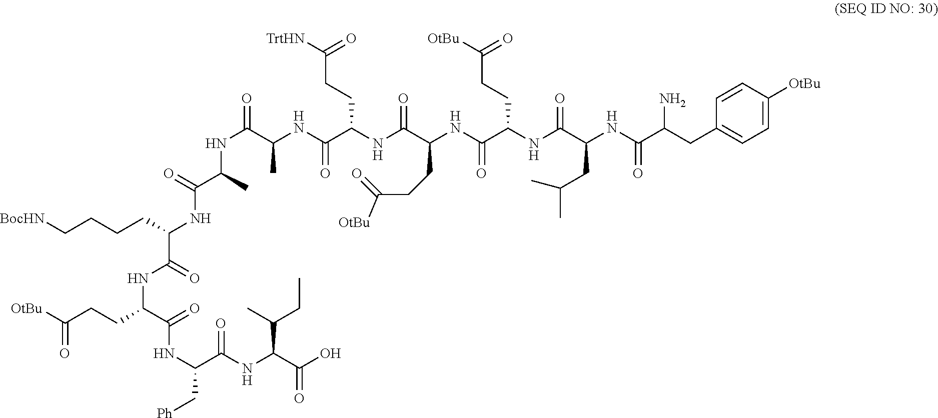

or a salt thereof.

### Fragment 2C

Also provided herein is a peptide having a sequence of Leu-Glu-Glu-Gln-Ala-Ala-Lys-Glu-Phe-Ile-Ala (SEQ ID NO: 17) (“Fragment 2C”). Certain aspects provide a protected variant of Fragment 2C, wherein at least one of the Glu, Gln, and Lys residues is protected with a protecting group.

In some embodiments, the disclosure provides a protected variant of Fragment 2C, wherein:
the side chain of the Glu residue at position 2, position 3, or position 8 is protected with a tert-butyl ester (OtBu), O-2-phenylisopropyl ester (OPp), or allyl ester (OAll) protecting group; and/or
the side chain of the Gln residue at position 4 is protected with a trityl (Trt), benzyloxymethyl (Bom), xanthenyl (Xan), or p-methoxybenzyl (PMB) protecting group; and/or
the side chain of the Lys residue at position 7 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), 4-methyltrityl (Mtt), or benzyloxycarbonyl (Cbz) protecting group.

In some embodiments, the N-terminus of the Leu residue at position 1 is protected with a 9-fluorenylmethoxycarbonyl (Fmoc) protecting group.

In some embodiments, the disclosure provides a protected variant of Fragment 2C, wherein:
the side chain of the Glu residue at position 2, position 3, or position 8 is protected with a tert-butyl ester (OtBu) protecting group; and/or
the side chain of the Gln residue at position 4 is protected with a trityl (Trt) protecting group; and/or
the side chain of the Lys residue at position 7 is protected with a tert-butyloxycarbonyl (Boc), 1-(4,4-dimethyl-2,6-dioxocyclohex-1-ylidene)ethyl (Dde), or allyloxycarbonyl (Alloc) protecting group.

In some embodiments, the N-terminus of the Leu residue at position 1 is protected with a 9-fluorenylmethoxycarbonyl (Fmoc) protecting group.

In some embodiments, the disclosure provides a protected variant of Fragment 2C, wherein:
the side chain of the Glu residue at position 2, position 3, or position 8 is protected with an allyl ester (OAll), benzyl ester (OBn), or cyclohexyl ester (OcHex) protecting group; and/or
the side chain of the Lys residue at position 7 is protected with a benzyloxycarbonyl (Cbz), 2-chlorobenzyloxycarbonyl(2-Cl—Z), or allyloxycarbonyl (Alloc) protecting group.

In some embodiments, the N-terminus of the Leu residue at position 1 is protected with a tert-butyloxycarbonyl (Boc) protecting group.

In some embodiments, the disclosure provides a protected variant of Fragment 2C, wherein:
the side chain of the Glu residue at position 2, position 3, or position 8 is protected with a benzyl ester (OBn) or allyl ester (OAll) protecting group; and/or
the side chain of the Lys residue at position 7 is protected with a benzyloxycarbonyl (Cbz) or 2-chlorobenzyloxycarbonyl(2-Cl—Z) protecting group.

In some embodiments, the N-terminus of the Leu residue at position 1 is protected with a tert-butyloxycarbonyl (Boc) protecting group.

Certain aspects provide a protected variant of Fragment 2C, wherein the Glu residue at position 2 is protected as a t-butyl ester (OtBu). Certain aspects provide a protected variant of Fragment 2C, wherein the Glu residue at position 3 is protected as a t-butyl ester (OtBu). Certain aspects provide a protected variant of Fragment 2C, wherein the Gln residue at position 4 is protected as a triphenylmethyl amide (Trt). Certain aspects provide a protected variant of Fragment 2C, wherein the Lys residue at position 7 is protected as a t-butyloxy carbonyl amide (Boc). Certain aspects provide a protected variant of Fragment 2C, wherein the Glu residue at position 8 is protected as a t-butyl ester (OtBu).

In some embodiments, the present disclosure provides a protected variant of Fragment 2C, wherein:
the Glu residue at position 2 is protected as a t-butyl ester (OtBu); and/or
the Glu residue at position 3 is protected as a t-butyl ester (OtBu); and/or
the Gln residue at position 4 is protected as a triphenylmethyl amide (Trt); and/or
the Lys residue at position 7 is protected as a t-butyloxy carbonyl amide (Boc); and/or
the Glu residue at position 8 is protected as a t-butyl ester (OtBu).

In some embodiments, the Leu residue at position 1 is protected with an Fmoc protecting group at the N-terminus.

In some embodiments, the present disclosure provides a protected variant of Fragment 2C, wherein:
the Glu residue at position 2 is protected as a t-butyl ester (OtBu);
the Glu residue at position 3 is protected as a t-butyl ester (OtBu);
the Gln residue at position 4 is protected as a triphenylmethyl amide (Trt);
the Lys residue at position 7 is protected as a t-butyloxy carbonyl amide (Boc); and
the Glu residue at position 8 is protected as a t-butyl ester (OtBu).

In some embodiments, the Leu residue at position 1 is protected with an Fmoc protecting group at the N-terminus.

In some embodiments, the protected variant of Fragment 2C is Leu-Glu(OtBu)-Glu(OtBu)-Gln(Trt)-Ala-Ala-Lys(Boc)-Glu(OtBu)-Phe-Ile-Ala-OH (SEQ ID NO: 31) or a salt thereof. In some embodiments, the protected variant of Fragment 2C is in the form of an acid-addition salt. In some embodiments, the protected variant of Fragment 2C is in the form of a carboxylate salt.

In some embodiments, the protected variant of Fragment 2C is Leu-Glu(OtBu)-Glu(OtBu)-Gln(Trt)-Ala-Ala-Lys(Boc)-Glu(OtBu)-Phe-Ile-Ala-OH, wherein the protected variant of Fragment 2C has a protected N-terminus (SEQ ID NO: 39), or a salt (e.g., a carboxylate salt) thereof. In some embodiments, the protected variant of Fragment 2C is Fmoc-Leu-Glu(OtBu)-Glu(OtBu)-Gln(Trt)-Ala-Ala-Lys(Boc)-Glu(OtBu)-Phe-Ile-Ala-OH (SEQ ID NO: 55) or a salt (e.g., a carboxylate salt) thereof.

In some embodiments, the protected variant of Fragment 2C is Leu-Glu(OtBu)-Glu(OtBu)-Gln(Trt)-Ala-Ala-Lys(Boc)-Glu(OtBu)-Phe-Ile-Ala, wherein the protected variant of Fragment 2C has a protected N-terminus (e.g., an Fmoc-protected N-terminus) and a protected C-terminus (SEQ ID NO: 105).

In some embodiments, the protected variant of Fragment 2C is Leu-Glu(OtBu)-Glu(OtBu)-Gln(Trt)-Ala-Ala-Lys(Boc)-Glu(OtBu)-Phe-Ile-Ala, wherein the protected variant of Fragment 2C has a protected C-terminus (SEQ ID NO: 47), or a salt (e.g., an acid-addition salt) thereof.

Certain aspects provide a protected variant of Fragment 2C, having a structure of

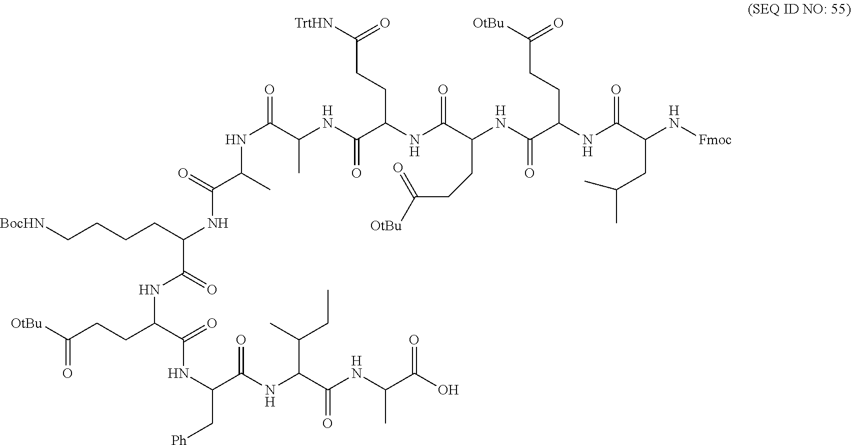

or a salt (e.g., a carboxylate salt) thereof.

### Fragment 3

Also provided herein is a peptide having a sequence of Trp-Leu-Val-Lys-Gly-Gly-Gly-Gly-Gly-Gly-Gly (SEQ ID NO: 3) (“Fragment 3”). Certain aspects provide a protected variant of Fragment 3, wherein at least one of the Trp and Lys residues is protected with a protecting group.

In some embodiments, the disclosure provides a protected variant of Fragment 3, wherein:
the side chain of the Trp residue at position 1 is protected with a tert-butyloxycarbonyl (Boc) protecting group; and/or
the side chain of the Lys residue at position 4 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), 4-methyltrityl (Mtt), or benzyloxycarbonyl (Cbz) protecting group.

In some embodiments, the N-terminus of the Trp residue at position 1 is protected with a 9-fluorenylmethoxycarbonyl (Fmoc) protecting group.

In some embodiments, the disclosure provides a protected variant of Fragment 3, wherein:
the side chain of the Trp residue at position 1 is protected with a tert-butyloxycarbonyl (Boc) protecting group; and/or
the side chain of the Lys residue at position 4 is protected with a tert-butyloxycarbonyl (Boc), 1-(4,4-dimethyl-2,6-dioxocyclohex-1-ylidene)ethyl (Dde), or allyloxycarbonyl (Alloc) protecting group.

In some embodiments, the N-terminus of the Trp residue at position 1 is protected with a 9-fluorenylmethoxycarbonyl (Fmoc) protecting group.

In some embodiments, the disclosure provides a protected variant of Fragment 3, wherein:
the side chain of the Trp residue at position 1 is protected with a formyl (For) protecting group; and/or
the side chain of the Lys residue at position 4 is protected with a benzyloxycarbonyl (Cbz), 2-chlorobenzyloxycarbonyl(2-Cl—Z), or allyloxycarbonyl (Alloc) protecting group.

In some embodiments, the N-terminus of the Trp residue at position 1 is protected with a tert-butyloxycarbonyl (Boc) protecting group.

In some embodiments, the disclosure provides a protected variant of Fragment 3, wherein:
the side chain of the Trp residue at position 1 is protected with a formyl (For) protecting group; and/or
the side chain of the Lys residue at position 4 is protected with a benzyloxycarbonyl (Cbz) or 2-chlorobenzyloxycarbonyl(2-Cl—Z) protecting group.

In some embodiments, the N-terminus of the Trp residue at position 1 is protected with a tert-butyloxycarbonyl (Boc) protecting group.

Certain aspects provide a protected variant of Fragment 3, wherein the Trp residue is protected with an Fmoc protecting group at the N-terminus. Certain aspects provide a protected variant of Fragment 3, wherein the Trp residue is protected as a t-butyloxy carbonyl amide (Boc) at the Trp side chain. Certain aspects provide a protected variant of Fragment 3, wherein the Lys residue at position 4 is protected as t-butyloxy carbonyl amide (Boc).

In some embodiments, the present disclosure provides a protected variant of Fragment 3, wherein:
the Trp residue at position 1 is protected as a t-butyloxy carbonyl amide (Boc) at the Trp side chain; and/or
the Lys residue at position 4 is protected as t-butyloxy carbonyl amide (Boc).

In some embodiments, the Trp residue at position 1 is protected with an Fmoc protecting group at the N-terminus.

In some embodiments, the present disclosure provides a protected variant of Fragment 3, wherein:
the Trp residue at position 1 is protected as a t-butyloxy carbonyl amide (Boc) at the Trp side chain; and
the Lys residue at position 4 is protected as t-butyloxy carbonyl amide (Boc).

In some embodiments, the Trp residue at position 1 is protected with an Fmoc protecting group at the N-terminus.

In some embodiments, the protected variant of Fragment 3 is Trp(Boc)-Leu-Val-Lys(Boc)-Gly-Gly-Gly-Gly-Gly-Gly-Gly-OH (SEQ ID NO: 32) or a salt thereof. In some embodiments, the protected variant of Fragment 3 is in the form of an acid-addition salt. In some embodiments, the protected variant of Fragment 3 is in the form of a carboxylate salt.

In some embodiments, the protected variant of Fragment 3 is Trp(Boc)-Leu-Val-Lys(Boc)-Gly-Gly-Gly-Gly-Gly-Gly-Gly-OH, wherein the protected variant of Fragment 3 has a protected N-terminus (SEQ ID NO: 40), or a salt (e.g., a carboxylate salt) thereof. In some embodiments, the protected variant of Fragment 3 is Fmoc-Trp(Boc)-Leu-Val-Lys(Boc)-Gly-Gly-Gly-Gly-Gly-Gly-Gly-OH (SEQ ID NO: 56) or a salt (e.g., a carboxylate salt) thereof.

In some embodiments, the protected variant of Fragment 3 is Trp(Boc)-Leu-Val-Lys(Boc)-Gly-Gly-Gly-Gly-Gly-Gly-Gly, wherein the protected variant of Fragment 3 has a protected N-terminus (e.g., an Fmoc-protected N-terminus) and a protected C-terminus (SEQ ID NO: 106).

In some embodiments, the protected variant of Fragment 3 is Trp(Boc)-Leu-Val-Lys(Boc)-Gly-Gly-Gly-Gly-Gly-Gly-Gly, wherein the protected variant of Fragment 3 has a protected C-terminus (SEQ ID NO: 48), or a salt (e.g., an acid-addition salt) thereof.

Certain aspects provide a protected variant of Fragment 3, having a structure of

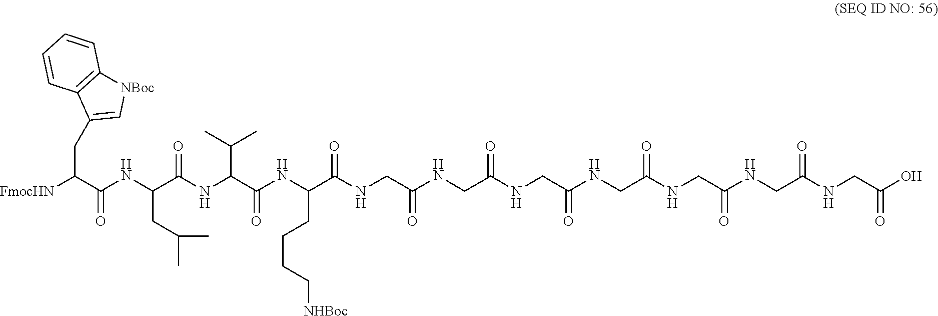

or a salt (e.g., a carboxylate salt) thereof.

In some embodiments, the protected variant of Fragment 3 has the structure:

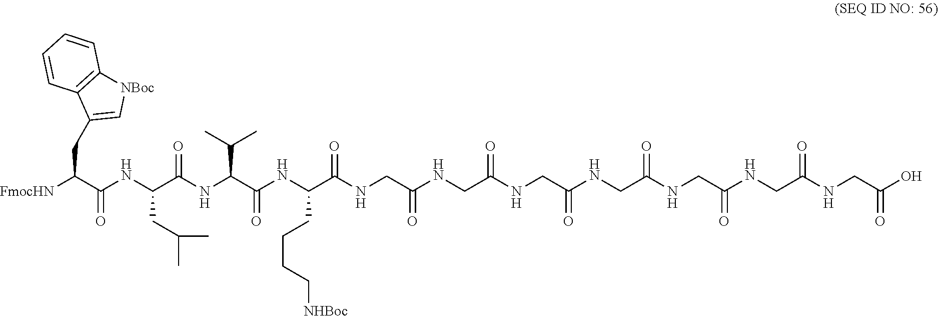

or a salt (e.g., a carboxylate salt) thereof.

### Fragment 3A

Also provided herein is a peptide having a sequence of Ala-Trp-Leu-Val-Lys-Gly-Gly-Gly-Gly-Gly-Gly-Gly (SEQ ID NO: 13) (“Fragment 3A”). Certain aspects provide a protected variant of Fragment 3A, wherein at least one of the Ala, Trp, and Lys residues is protected with a protecting group.

In some embodiments, the disclosure provides a protected variant of Fragment 3A, wherein:
the side chain of the Trp residue at position 2 is protected with a tert-butyloxycarbonyl (Boc) protecting group; and/or
the side chain of the Lys residue at position 5 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), 4-methyltrityl (Mtt), or benzyloxycarbonyl (Cbz) protecting group.

In some embodiments, the N-terminus of the Ala residue at position 1 is protected with a 9-fluorenylmethoxycarbonyl (Fmoc) protecting group.

In some embodiments, the disclosure provides a protected variant of Fragment 3A, wherein:
the side chain of the Trp residue at position 2 is protected with a tert-butyloxycarbonyl (Boc) protecting group; and/or
the side chain of the Lys residue at position 5 is protected with a tert-butyloxycarbonyl (Boc), 1-(4,4-dimethyl-2,6-dioxocyclohex-1-ylidene)ethyl (Dde), or allyloxycarbonyl (Alloc) protecting group.

In some embodiments, the N-terminus of the Ala residue at position 1 is protected with a 9-fluorenylmethoxycarbonyl (Fmoc) protecting group.

In some embodiments, the disclosure provides a protected variant of Fragment 3A, wherein:
the side chain of the Trp residue at position 2 is protected with a formyl (For) protecting group; and/or
the side chain of the Lys residue at position 5 is protected with a benzyloxycarbonyl (Cbz), 2-chlorobenzyloxycarbonyl(2-Cl—Z), or allyloxycarbonyl (Alloc) protecting group.

In some embodiments, the N-terminus of the Ala residue at position 1 is protected with a tert-butyloxycarbonyl (Boc) protecting group.

In some embodiments, the disclosure provides a protected variant of Fragment 3A, wherein:
the side chain of the Trp residue at position 2 is protected with a formyl (For) protecting group; and/or
the side chain of the Lys residue at position 5 is protected with a benzyloxycarbonyl (Cbz) or 2-chlorobenzyloxycarbonyl(2-Cl—Z) protecting group.

In some embodiments, the N-terminus of the Ala residue at position 1 is protected with a tert-butyloxycarbonyl (Boc) protecting group.

Certain aspects provide a protected variant of Fragment 3A, wherein the Ala residue at position 1 is protected with an Fmoc protecting group at the N-terminus. Certain aspects provide a protected variant of Fragment 3A, wherein the Trp residue at position 2 is protected as a t-butyloxy carbonyl amide (Boc) at the Trp side chain. Certain aspects provide a protected variant of Fragment 3A, wherein the Lys residue at position 5 is protected as t-butyloxy carbonyl amide (Boc).

In some embodiments, the present disclosure provides a protected variant of Fragment 3A, wherein:
the Ala residue at position 1 is protected with an Fmoc protecting group at the N-terminus; and/or
the Trp residue at position 2 is protected as a t-butyloxy carbonyl amide (Boc); and/or
the Lys residue at position 5 is protected as t-butyloxy carbonyl amide (Boc).

In some embodiments, the present disclosure provides a protected variant of Fragment 3A, wherein:
the Trp residue at position 2 is protected as a t-butyloxy carbonyl amide (Boc); and
the Lys residue at position 5 is protected as t-butyloxy carbonyl amide (Boc).

In some embodiments, the Ala residue at position 1 is protected with an Fmoc protecting group at the N-terminus.

In some embodiments, the protected variant of Fragment 3A is Ala-Trp(Boc)-Leu-Val-Lys(Boc)-Gly-Gly-Gly-Gly-Gly-Gly-Gly-OH (SEQ ID NO: 33) or a salt thereof. In some embodiments, the protected variant of Fragment 3A is in the form of an acid-addition salt. In some embodiments, the protected variant of Fragment 3A is in the form of a carboxylate salt.

In some embodiments, the protected variant of Fragment 3A is Ala-Trp(Boc)-Leu-Val-Lys(Boc)-Gly-Gly-Gly-Gly-Gly-Gly-Gly-OH, wherein the protected variant of Fragment 3A has a protected N-terminus (SEQ ID NO: 41), or a salt (e.g., a carboxylate salt) thereof. In some embodiments, the protected variant of Fragment 3A is Fmoc-Ala-Trp(Boc)-Leu-Val-Lys(Boc)-Gly-Gly-Gly-Gly-Gly-Gly-Gly-OH (SEQ ID NO: 57) or a salt (e.g., a carboxylate salt) thereof.

In some embodiments, the protected variant of Fragment 3A is Ala-Trp(Boc)-Leu-Val-Lys(Boc)-Gly-Gly-Gly-Gly-Gly-Gly-Gly, wherein the protected variant of Fragment 3 has a protected N-terminus (e.g., an Fmoc-protected N-terminus) and a protected C-terminus (SEQ ID NO: 107).

In some embodiments, the protected variant of Fragment 3A is Ala-Trp(Boc)-Leu-Val-Lys(Boc)-Gly-Gly-Gly-Gly-Gly-Gly-Gly, wherein the protected variant of Fragment 3 has a protected C-terminus (SEQ ID NO: 49), or a salt (e.g., an acid-addition salt) thereof.

Certain aspects provide a protected variant of Fragment 3A, having a structure of

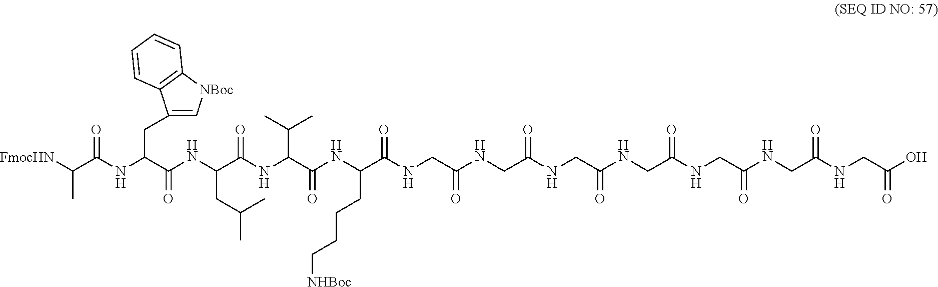

or a salt (e.g., a carboxylate salt) thereof.

In some embodiments, the protected variant of Fragment 3A has the structure

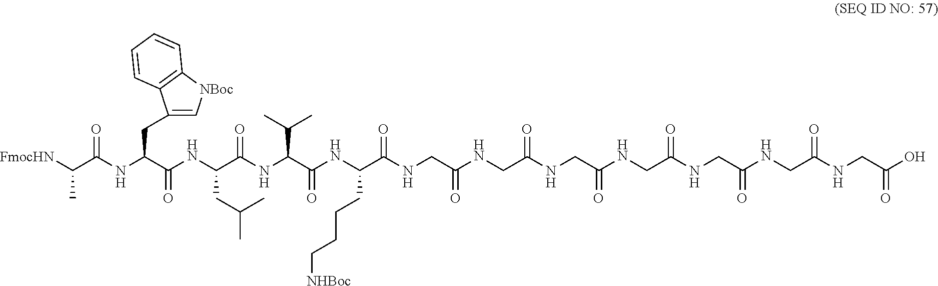

or a salt (e.g., a carboxylate salt) thereof.

### Fragment 4

Also provided herein is a peptide having a sequence of Ser-Gly-Gly-Gly-Gly-Ser-Gly-Gly-Gly-Gly-Ser-Lys (SEQ ID NO: 4) (“Fragment 4”). Certain aspects provide a protected variant of Fragment 4, wherein at least one of the Ser and Lys residues is protected with a protecting group. In addition, certain aspects provide a protected variant of Fragment 4, wherein the Lys residue at position 12 is protected with a side chain protecting group that can be selectively removed (i.e., is orthogonal to any other side chain or termini protecting group(s), such as, e.g., a 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), benzyloxycarbonyl (Cbz), or chlorobenzyloxycarbonyl(2-Cl—Z) protecting group) to enable selective modification of the &-amino group of the C-terminal Lys residue.

In some embodiments, the disclosure provides a protected variant of Fragment 4, wherein:
the side chain of the Ser residue at position 1, position 6, or position 11 is protected with a tert-butyl (tBu), O-trityl (OTrt), O-allyl (OAll), or pseudoproline (YPro) protecting group; and/or
the side chain of the Lys residue at position 12 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), 4-methyltrityl (Mtt), or benzyloxycarbonyl (Cbz) protecting group.

In some embodiments, the N-terminus of the Ser residue at position 1 is protected with a 9-fluorenylmethoxycarbonyl (Fmoc) protecting group.

In some embodiments, the disclosure provides a protected variant of Fragment 4, wherein:
the side chain of the Ser residue at position 1, position 6, or position 11 is protected with a tert-butyl (tBu) or O-trityl (OTrt) protecting group; and/or
the side chain of the Lys residue at position 12 is protected with a tert-butyloxycarbonyl (Boc), 1-(4,4-dimethyl-2,6-dioxocyclohex-1-ylidene)ethyl (Dde), or allyloxycarbonyl (Alloc) protecting group.

In some embodiments, the N-terminus of the Ser residue at position 1 is protected with a 9-fluorenylmethoxycarbonyl (Fmoc) protecting group.

In some embodiments, the disclosure provides a protected variant of Fragment 4, wherein:
the N-terminus of the Ser residue at position 1 is protected with a 9-fluorenylmethoxycarbonyl (Fmoc) protecting group;
the side chain of the Ser residue at position 1, position 6, or position 11 is protected with a tert-butyl (tBu), O-trityl (OTrt), or pseudoproline (YPro) protecting group; and
the side chain of the Lys residue at position 12 is protected with an allyloxycarbonyl (Alloc) protecting group.

In some embodiments, the disclosure provides a protected variant of Fragment 4, wherein:
the N-terminus of the Ser residue at position 1 is protected with a 9-fluorenylmethoxycarbonyl (Fmoc) protecting group;
the side chain of the Ser residue at position 1, position 6, or position 11 is protected with a tert-butyl (tBu), O-trityl (OTrt), O-allyl (OAll), or pseudoproline (YPro) protecting group; and
the side chain of the Lys residue at position 12 is protected with a 1-(4,4-dimethyl-2,6-dioxocyclohex-1-ylidene)ethyl (Dde) or 1-(4,4-dimethyl-2,6-dioxocyclohex-1-ylidene)-3-methylbutyl (ivDde) protecting group.

In some embodiments, the disclosure provides a protected variant of Fragment 4, wherein:
the N-terminus of the Ser residue at position 1 is protected with a 9-fluorenylmethoxycarbonyl (Fmoc) protecting group;
the side chain of the Ser residue at position 1, position 6, or position 11 is protected with a tert-butyl (tBu), O-trityl (OTrt), O-allyl (OAll), or pseudoproline (YPro) protecting group; and
the side chain of the Lys residue at position 12 is protected with a benzyloxycarbonyl (Cbz) or chlorobenzyloxycarbonyl(2-Cl—Z) protecting group.

In some embodiments, the disclosure provides a protected variant of Fragment 4, wherein:
the side chain of the Ser residue at position 1, position 6, or position 11 is protected with a p-bromobenzyloxycarbonyl (Br-Z), O-benzyl (OBn), or O-allyl (OAll) protecting group; and/or
the side chain of the Lys residue at position 12 is protected with a benzyloxycarbonyl (Cbz), 2-chlorobenzyloxycarbonyl(2-Cl—Z), or allyloxycarbonyl (Alloc) protecting group.

In some embodiments, the N-terminus of the Ser residue at position 1 is protected with a tert-butyloxycarbonyl (Boc) protecting group.

In some embodiments, the disclosure provides a protected variant of Fragment 4, wherein:
the side chain of the Ser residue at position 1, position 6, or position 11 is protected with an O-benzyl (OBn) protecting group; and/or
the side chain of the Lys residue at position 12 is protected with a benzyloxycarbonyl (Cbz) or 2-chlorobenzyloxycarbonyl(2-Cl—Z) protecting group.

In some embodiments, the N-terminus of the Ser residue at position 1 is protected with a tert-butyloxycarbonyl (Boc) protecting group.

In some embodiments, the disclosure provides a protected variant of Fragment 4, wherein:
the N-terminus of the Ser residue at position 1 is protected with a tert-butyloxycarbonyl (Boc) protecting group;
the side chain of the Ser residue at position 1, position 6, or position 11 is protected with a p-bromobenzyloxycarbonyl (Br-Z) or O-benzyl (OBn) protecting group; and
the side chain of the Lys residue at position 12 is protected with an allyloxycarbonyl (Alloc) protecting group.

In some embodiments, the disclosure provides a protected variant of Fragment 4, wherein:
the N-terminus of the Ser residue at position 1 is protected with a tert-butyloxycarbonyl (Boc) protecting group;
the side chain of the Ser residue at position 1, position 6, or position 11 is protected with a p-bromobenzyloxycarbonyl (Br-Z), O-benzyl (OBn), or O-allyl (OAll) protecting group; and
the side chain of the Lys residue at position 12 is protected with a 1-(4,4-dimethyl-2,6-dioxocyclohex-1-ylidene)ethyl (Dde) or 1-(4,4-dimethyl-2,6-dioxocyclohex-1-ylidene)-3-methylbutyl (ivDde) protecting group.

In some embodiments, the disclosure provides a protected variant of Fragment 4, wherein:
the N-terminus of the Ser residue at position 1 is protected with a tert-butyloxycarbonyl (Boc) protecting group;
the side chain of the Ser residue at position 1, position 6, or position 11 is protected with an O-allyl (OAll) protecting group; and
the side chain of the Lys residue at position 12 is protected with a benzyloxycarbonyl (Cbz) or chlorobenzyloxycarbonyl(2-Cl—Z) protecting group.

Certain aspects provide a protected variant of Fragment 4, wherein the Ser residue at position 1 is protected with an Fmoc protecting group at the N-terminus. Certain aspects provide a protected variant of Fragment 4, wherein the Ser residue at position 1 is protected as a t-butyl ether (tBu) on the Ser side chain. Certain aspects describe a protected variant of Fragment 4, wherein the Ser residue at position 6 is protected as a t-butyl ether (tBu). Certain aspects provide a protected variant of Fragment 4, wherein the Ser residue at position 11 is protected as a t-butyl ether (tBu). Certain aspects provide a protected variant of Fragment 4, wherein the Lys residue at position 12 is protected as a N-ε-1-(4,4-dimethyl-2,6-dioxocyclohex-1-ylidene)-3-methylbutyl amine (ivDde).

In some embodiments, the present disclosure provides a protected variant of Fragment 4, wherein:
the Ser residue at position 1 is protected with an Fmoc protecting group at the N-terminus; and/or
the Ser residue at position 1 is protected as a t-butyl ether (tBu) on the Ser side chain; and/or
the Ser residue at position 6 is protected as a t-butyl ether (tBu); and/or
the Ser residue at position 11 is protected as a t-butyl ether (tBu); and/or
the Lys residue at position 12 is protected as a N-ε-1-(4,4-dimethyl-2,6-dioxocyclohex-1-ylidene)-3-methylbutyl amine (ivDde).

In some embodiments, the present disclosure provides a protected variant of Fragment 4, wherein:
the Ser residue at position 1 is protected as a t-butyl ether (tBu) on the Ser side chain; the Ser residue at position 6 is protected as a t-butyl ether (tBu);
the Ser residue at position 11 is protected as a t-butyl ether (tBu); and
the Lys residue at position 12 is protected as a N-ε-1-(4,4-dimethyl-2,6-dioxocyclohex-1-ylidene)-3-methylbutyl amine (ivDde).

In some embodiments, the Ser residue at position 1 is protected with an Fmoc protecting group at the N-terminus.

In some embodiments, the protected variant of Fragment 4 does not have a free C-terminal carboxyl group. In some embodiments, the protected variant of Fragment 4 has a protected C-terminus. In some embodiments, the C-terminus of the protected variant of Fragment 4 is amidated.

In some embodiments, the protected variant of Fragment 4 is Ser(tBu)-Gly-Gly-Gly-Gly-Ser(tBu)-Gly-Gly-Gly-Gly-Ser(tBu)-Lys(ivDde), wherein the protected variant of Fragment 4 does not have a free C-terminal carboxyl group (SEQ ID NO: 61), or a salt (e.g., an acid-addition salt) thereof.

In some embodiments, the protected variant of Fragment 4 is Ser(tBu)-Gly-Gly-Gly-Gly-Ser(tBu)-Gly-Gly-Gly-Gly-Ser(tBu)-Lys(ivDde), wherein the protected variant of Fragment 4 has a protected C-terminus (SEQ ID NO: 108), or a salt (e.g., an acid-addition salt) thereof. In some embodiments, the protected variant of Fragment 4 is Ser(tBu)-Gly-Gly-Gly-Gly-Ser(tBu)-Gly-Gly-Gly-Gly-Ser(tBu)-Lys(ivDde)-NH₂ (SEQ ID NO: 109) or a salt (e.g., an acid-addition salt) thereof.

In some embodiments, the protected variant of Fragment 4 is Ser(tBu)-Gly-Gly-Gly-Gly-Ser(tBu)-Gly-Gly-Gly-Gly-Ser(tBu)-Lys(ivDde), wherein the protected variant of Fragment 4 has a protected N-terminus and does not have a free C-terminal carboxyl group (SEQ ID NO: 68). In some embodiments, the protected variant of Fragment 4 is Fmoc-Ser(tBu)-Gly-Gly-Gly-Gly-Ser(tBu)-Gly-Gly-Gly-Gly-Ser(tBu)-Lys(ivDde), wherein the protected variant of Fragment 4 does not have a free C-terminal carboxyl group (SEQ ID NO: 76). In some embodiments, the protected variant of Fragment 4 is Trt-Ser(tBu)-Gly-Gly-Gly-Gly-Ser(tBu)-Gly-Gly-Gly-Gly-Ser(tBu)-Lys(ivDde), wherein the protected variant of Fragment 4 does not have a free C-terminal carboxyl group (SEQ ID NO: 113).

In some embodiments, the protected variant of Fragment 4 is Ser(tBu)-Gly-Gly-Gly-Gly-Ser(tBu)-Gly-Gly-Gly-Gly-Ser(tBu)-Lys(ivDde), wherein the protected variant of Fragment 4 has a protected N-terminus and a protected C-terminus (SEQ ID NO: 114). In some embodiments, the protected variant of Fragment 4 is Fmoc-Ser(tBu)-Gly-Gly-Gly-Gly-Ser(tBu)-Gly-Gly-Gly-Gly-Ser(tBu)-Lys(ivDde), wherein the protected variant of Fragment 4 has a protected C-terminus (SEQ ID NO: 115). In some embodiments, the protected variant of Fragment 4 is Trt-Ser(tBu)-Gly-Gly-Gly-Gly-Ser(tBu)-Gly-Gly-Gly-Gly-Ser(tBu)-Lys(ivDde), wherein the protected variant of Fragment 4 has a protected C-terminus (SEQ ID NO: 116).

In some embodiments, the protected variant of Fragment 4 is Ser(tBu)-Gly-Gly-Gly-Gly-Ser(tBu)-Gly-Gly-Gly-Gly-Ser(tBu)-Lys(ivDde)-NH₂, wherein the protected variant of Fragment 4 has a protected N-terminus (SEQ ID NO: 110). In some embodiments, the protected variant of Fragment 4 is Fmoc-Ser(tBu)-Gly-Gly-Gly-Gly-Ser(tBu)-Gly-Gly-Gly-Gly-Ser(tBu)-Lys(ivDde)-NH₂ (SEQ ID NO: 111). In some embodiments, the protected variant of Fragment 4 is Trt-Ser(tBu)-Gly-Gly-Gly-Gly-Ser(tBu)-Gly-Gly-Gly-Gly-Ser(tBu)-Lys(ivDde)-NH₂ (SEQ ID NO: 112).

Certain aspects provide a protected variant of Fragment 4, having a structure of

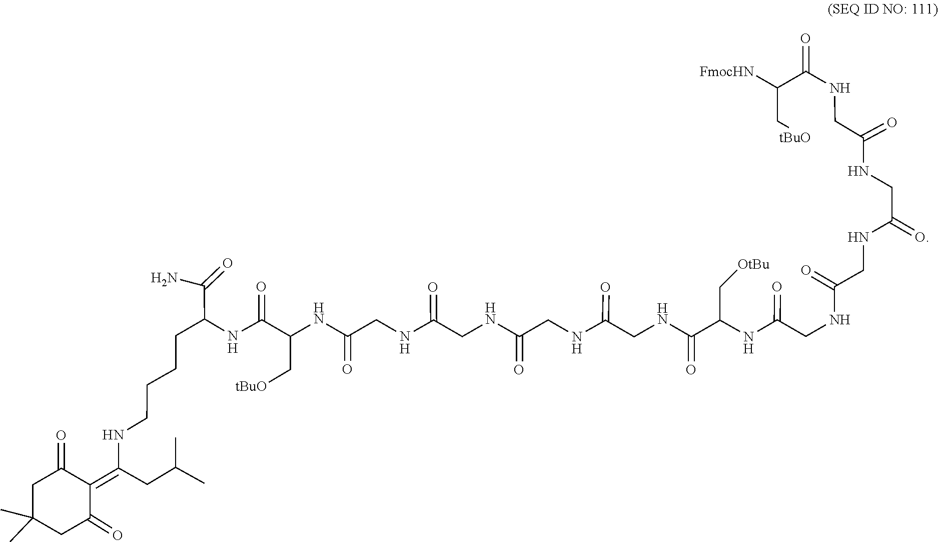

In some embodiments, the protected variant of Fragment 4 has the structure

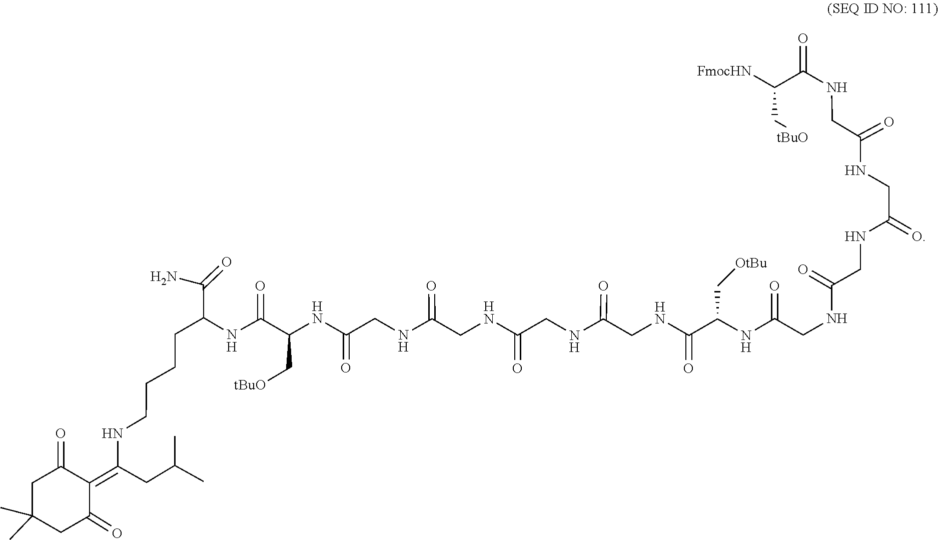

Certain aspects provide a conjugate of Fragment 4 or a protected variant thereof, wherein the Lys residue at position 12 is coupled to a solid support. Solid supports for solid phase synthesis are known in the art. In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing.

In some embodiments, the conjugate further comprises a linker through which the peptide is attached to the solid support (e.g., the resin material). Linkers for solid support are known in the art. In some embodiments, the linker is a rink-amide linker or a Sieber-amide linker.

In some embodiments, the solid support is a swellable resin material, and the conjugate further comprises a linker through which the peptide is attached to the swellable resin material.

In some embodiments, the solid support is a swellable resin material, and the conjugate further comprises a linker through which the peptide is attached to the swellable resin material, wherein the linker is a rink-amide linker. In some embodiments, the solid support is a swellable resin material, and the conjugate further comprises a linker through which the peptide is attached to the swellable resin material, wherein the linker is a Sieber-amide linker.

In some embodiments, the solid support is a Rink amide, peptide amide linker (PAL), safety-catch amide linker (SCAL), or Sieber amide resin. In some embodiments, the N-terminus of the conjugate is protected with a 9-fluorenylmethoxycarbonyl (Fmoc) protecting group, and the solid support is a Rink amide, peptide amide linker (PAL), safety-catch amide linker (SCAL), or Sieber amide resin.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin, a 2-chlorodiphenylmethyl resin, a 4-(hydroxymethyl) phenoxyacetic acid (HMPA) resin, a 4-(4-hydroxymethyl-3-methoxyphenoxy) butyric acid (HMPB) resin, or a resin comprising a dialkoxybenzyl-type benzyl alcohol linker on a polystyrene support. In some embodiments, the N-terminus of the conjugate is protected with a 9-fluorenylmethoxycarbonyl (Fmoc) protecting group, and the solid support is a Wang (p-benzyloxybenzyl) resin, a 2-chlorodiphenylmethyl resin, a 4-(hydroxymethyl) phenoxyacetic acid (HMPA) resin, a 4-(4-hydroxymethyl-3-methoxyphenoxy) butyric acid (HMPB) resin, or a resin comprising a dialkoxybenzyl-type benzyl alcohol linker on a polystyrene support.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol-grafted polystyrene. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

In some embodiments, the solid support is a Merrifield (benzyl ester) resin, a hydroxymethyl polystyrene (HMP) resin, a benzhydrylamine (BHA) resin, a p-methylbenzhydrylamine (MBHA) resin, or a phenylacetamidomethyl (PAM) resin. In some embodiments, the N-terminus of the conjugate is protected with a tert-butyloxycarbonyl (Boc) protecting group, and the solid support is a Merrifield (benzyl ester) resin, a hydroxymethyl polystyrene (HMP) resin, a benzhydrylamine (BHA) resin, a p-methylbenzhydrylamine (MBHA) resin, or a phenylacetamidomethyl (PAM) resin.

In some embodiments, the solid support is a Merrifield (benzyl ester) resin. In some embodiments, the solid support is a hydroxymethyl polystyrene (HMP) resin. In some embodiments, the solid support is a benzhydrylamine (BHA) resin. In some embodiments, the solid support is a p-Methylbenzhydrylamine (MBHA) resin. In some embodiments, the solid support is a phenylacetamidomethyl (PAM) resin.

Certain aspects provide a conjugate of a protected variant of Fragment 4, having a structure of

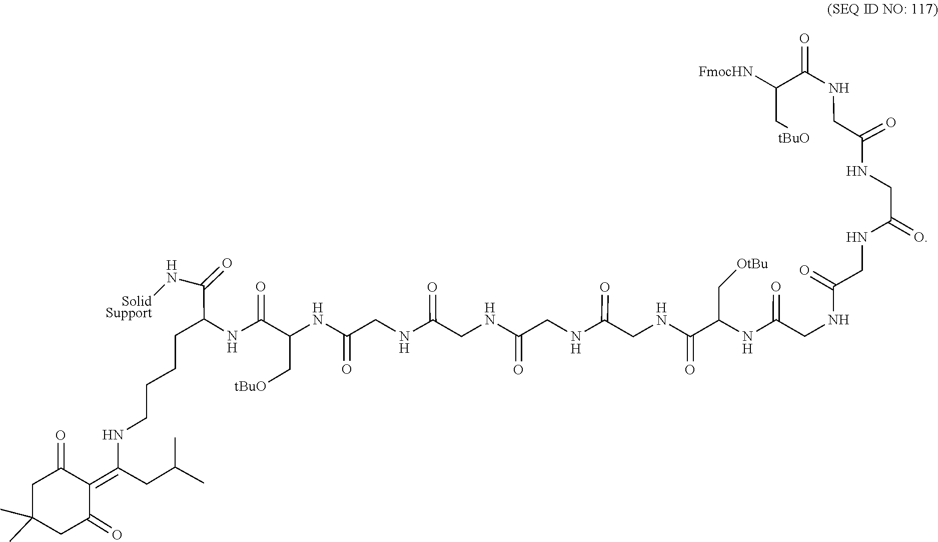

In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

In some embodiments, the conjugate of a protected variant of Fragment 4 has the structure

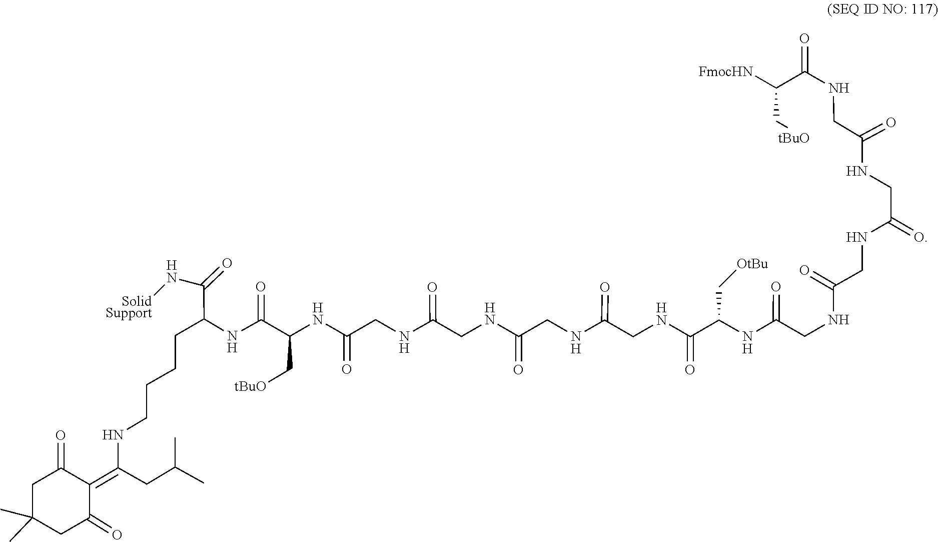

In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol-grafted polystyrene. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

### Fragment 5

Also provided herein is a peptide having a sequence of Ala-Trp-Leu-Val-Lys-Gly-Gly-Gly (SEQ ID NO: 5) (“Fragment 5”). Certain aspects provide a protected variant of Fragment 5, wherein at least one of the Ala, Trp and Lys residues is protected with a protecting group.

In some embodiments, the disclosure provides a protected variant of Fragment 5, wherein:
the side chain of the Trp residue at position 2 is protected with a tert-butyloxycarbonyl (Boc) protecting group; and/or
the side chain of the Lys residue at position 5 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), 4-methyltrityl (Mtt), or benzyloxycarbonyl (Cbz) protecting group.

In some embodiments, the N-terminus of the Ala residue at position 1 is protected with a 9-fluorenylmethoxycarbonyl (Fmoc) protecting group.

In some embodiments, the disclosure provides a protected variant of Fragment 5, wherein:
the side chain of the Trp residue at position 2 is protected with a tert-butyloxycarbonyl (Boc) protecting group; and/or
the side chain of the Lys residue at position 5 is protected with a tert-butyloxycarbonyl (Boc), 1-(4,4-dimethyl-2,6-dioxocyclohex-1-ylidene)ethyl (Dde), or allyloxycarbonyl (Alloc) protecting group.

In some embodiments, the N-terminus of the Ala residue at position 1 is protected with a 9-fluorenylmethoxycarbonyl (Fmoc) protecting group.

In some embodiments, the disclosure provides a protected variant of Fragment 5, wherein:
the side chain of the Trp residue at position 2 is protected with a formyl (For) protecting group; and/or
the side chain of the Lys residue at position 5 is protected with a benzyloxycarbonyl (Cbz), 2-chlorobenzyloxycarbonyl(2-Cl—Z), or allyloxycarbonyl (Alloc) protecting group.

In some embodiments, the N-terminus of the Ala residue at position 1 is protected with a tert-butyloxycarbonyl (Boc) protecting group.

In some embodiments, the disclosure provides a protected variant of Fragment 5, wherein:
the side chain of the Trp residue at position 2 is protected with a formyl (For) protecting group; and/or
the side chain of the Lys residue at position 5 is protected with a benzyloxycarbonyl (Cbz) or 2-chlorobenzyloxycarbonyl(2-Cl—Z) protecting group.

In some embodiments, the N-terminus of the Ala residue at position 1 is protected with a tert-butyloxycarbonyl (Boc) protecting group.

Certain aspects provide a protected variant of Fragment 5, wherein the Ala residue at position 1 is protected with an Fmoc protecting group at the N-terminus. Certain aspects provide a protected variant of Fragment 5, wherein the Ala residue at position 1 is protected with a Trt protecting group at the N-terminus. Certain aspects provide a protected variant of Fragment 5, wherein the Trp residue at position 2 is protected as t-butyloxy carbonyl amide (Boc). Certain aspects provide a protected variant of Fragment 5, wherein the Lys residue at position 5 is protected as t-butyloxy carbonyl amide (Boc).

In some embodiments, the present disclosure provides a protected variant of Fragment 5, wherein:
the Ala residue at position 1 is protected with an Fmoc protecting group or a Trt protecting group at the N-terminus; and/or
the Trp residue at position 2 is protected as t-butyloxy carbonyl amide (Boc); and/or
the Lys residue at position 5 is protected as t-butyloxy carbonyl amide (Boc).

In some embodiments, the present disclosure provides a protected variant of Fragment 5, wherein:
the Trp residue at position 2 is protected as t-butyloxy carbonyl amide (Boc); and
the Lys residue at position 5 is protected as t-butyloxy carbonyl amide (Boc).

In some embodiments, the Ala residue at position 1 is protected with an Fmoc protecting group at the N-terminus.

In some embodiments, the present disclosure provides a protected variant of Fragment 5, wherein:
the Trp residue at position 2 is protected as t-butyloxy carbonyl amide (Boc); and
the Lys residue at position 5 is protected as t-butyloxy carbonyl amide (Boc).

In some embodiments, the Ala residue at position 1 is protected with a Trt protecting group at the N-terminus.

In some embodiments, the protected variant of Fragment 5 is Ala-Trp(Boc)-Leu-Val-Lys(Boc)-Gly-Gly-Gly-OH (SEQ ID NO: 34) or a salt thereof. In some embodiments, the protected variant of Fragment 5 is an acid-addition salt thereof. In some embodiments, the protected variant of Fragment 5 is a carboxylate salt thereof.

In some embodiments, the protected variant of Fragment 5 is Ala-Trp(Boc)-Leu-Val-Lys(Boc)-Gly-Gly-Gly-OH, wherein the protected variant of Fragment 5 has a protected N-terminus (SEQ ID NO: 42), or a salt (e.g., a carboxylate salt) thereof. In some embodiments, the protected variant of Fragment 5 is Fmoc-Ala-Trp(Boc)-Leu-Val-Lys(Boc)-Gly-Gly-Gly-OH (SEQ ID NO: 58) or a salt (e.g., a carboxylate salt) thereof. In some embodiments, the protected variant of Fragment 5 is Trt-Ala-Trp(Boc)-Leu-Val-Lys(Boc)-Gly-Gly-Gly-OH (SEQ ID NO: 59) or a salt (e.g., a carboxylate salt) thereof.

In some embodiments, the protected variant of Fragment 5 is Ala-Trp(Boc)-Leu-Val-Lys(Boc)-Gly-Gly-Gly, wherein the protected variant of Fragment 5 has a protected N-terminus (e.g., an Fmoc-protected or Trt-protected N-terminus) and a protected C-terminus (SEQ ID NO: 118).

In some embodiments, the protected variant of Fragment 5 is Ala-Trp(Boc)-Leu-Val-Lys(Boc)-Gly-Gly-Gly, wherein the protected variant of Fragment 5 has a protected C-terminus (SEQ ID NO: 50), or a salt (e.g., an acid-addition salt) thereof.

In some embodiments, the present disclosure provides a protected variant of Fragment 5, wherein the Trp residue at position 2 is protected as t-butyloxy carbonyl amide (Boc).

In some embodiments, the present disclosure provides a protected variant of Fragment 5, wherein:
the Ala residue at position 1 is protected with an Fmoc protecting group at the N-terminus; and
the Trp residue at position 2 is protected as t-butyloxy carbonyl amide (Boc).

In some embodiments, the protected variant of Fragment 5 is Ala-Trp(Boc)-Leu-Val-Lys-Gly-Gly-Gly-OH (SEQ ID NO: 35) or a salt thereof. In some embodiments, the protected variant of Fragment 5 is in the form of an acid-addition salt. In some embodiments, the protected variant of Fragment 5 is in the form of a carboxylate salt.

In some embodiments, the protected variant of Fragment 5 is Ala-Trp(Boc)-Leu-Val-Lys-Gly-Gly-Gly-OH, wherein the protected variant of Fragment 5 has a protected N-terminus (SEQ ID NO: 43), or a salt (e.g., a carboxylate salt) thereof. In some embodiments, the protected variant of Fragment 5 is Fmoc-Ala-Trp(Boc)-Leu-Val-Lys-Gly-Gly-Gly-OH (SEQ ID NO: 60) or a salt (e.g., a carboxylate salt) thereof.

In some embodiments, the protected variant of Fragment 5 is Ala-Trp(Boc)-Leu-Val-Lys-Gly-Gly-Gly, wherein the protected variant of Fragment 5 has a protected N-terminus (e.g., an Fmoc-protected N-terminus) and a protected C-terminus (SEQ ID NO: 119).

In some embodiments, the protected variant of Fragment 5 is Ala-Trp(Boc)-Leu-Val-Lys-Gly-Gly-Gly, wherein the protected variant of Fragment 5 has a protected C-terminus (SEQ ID NO: 51), or a salt (e.g., an acid-addition salt) thereof.

Certain aspects provide a protected variant of Fragment 5, having a structure of

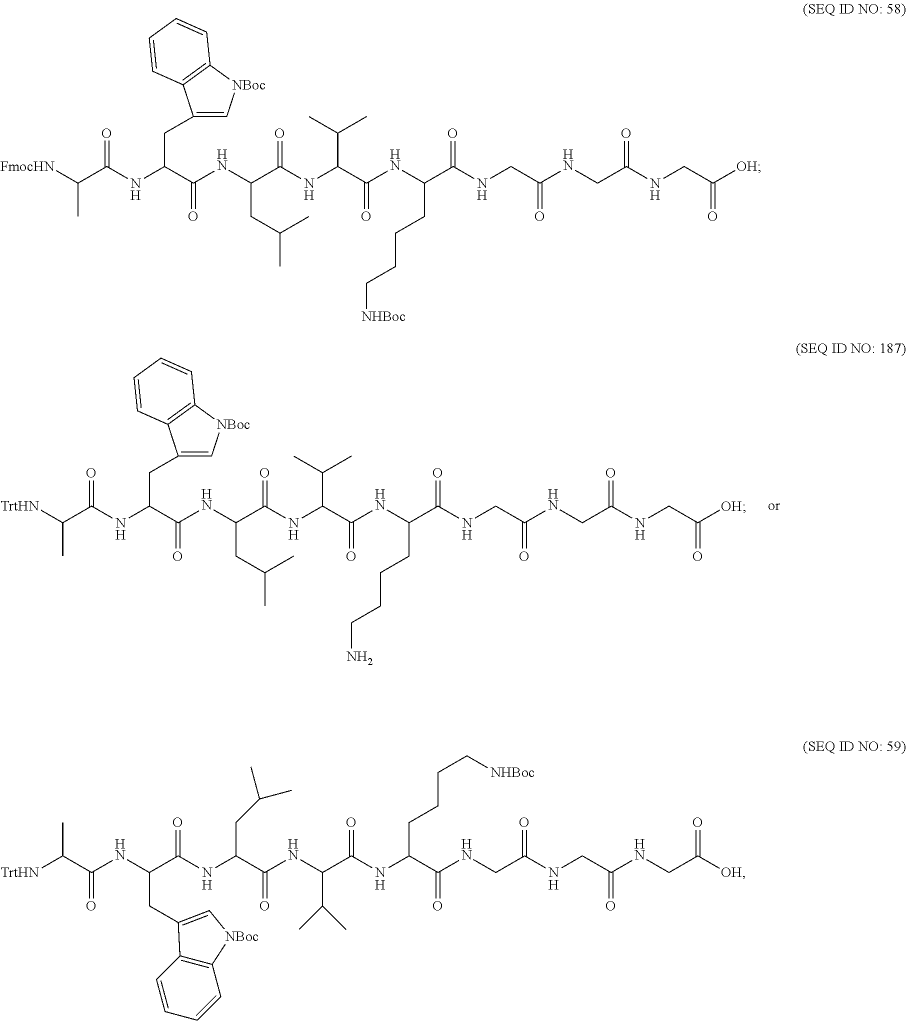

or a salt (e.g., a carboxylate salt) of any of the foregoing.

In some embodiments, the protected variant of Fragment 5 has the structure

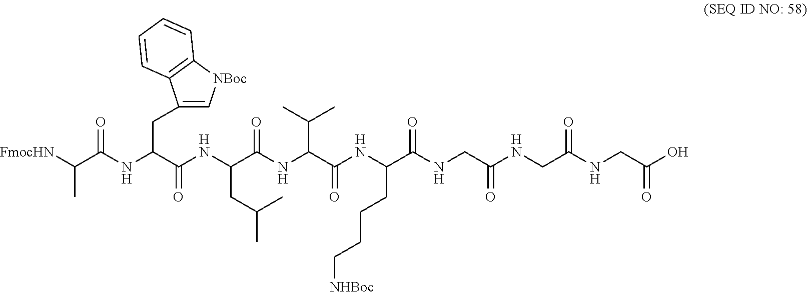

or a salt (e.g., a carboxylate salt) thereof.

In some embodiments, the protected variant of Fragment 5 has the structure

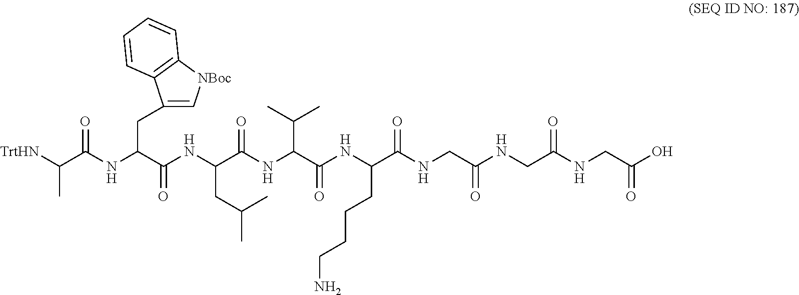

or a salt (e.g., a carboxylate salt) thereof.

In some embodiments, the protected variant of Fragment 5 has the structure

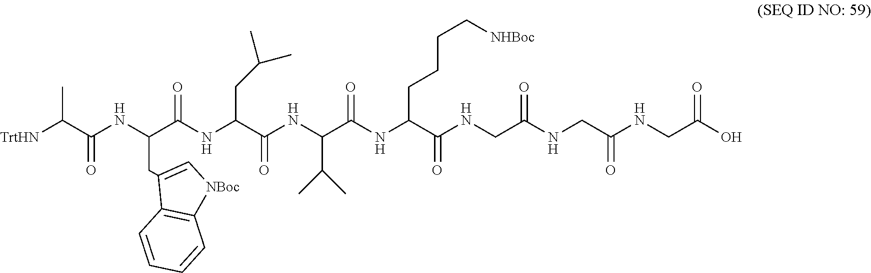

or a salt (e.g., a carboxylate salt) thereof.

In some embodiments, the protected variant of Fragment 5 has the structure

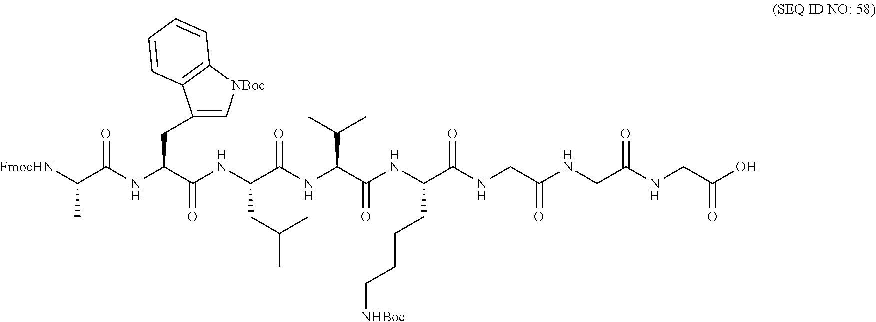

or a salt (e.g., a carboxylate salt) thereof.

In some embodiments, the protected variant of Fragment 5 has the structure

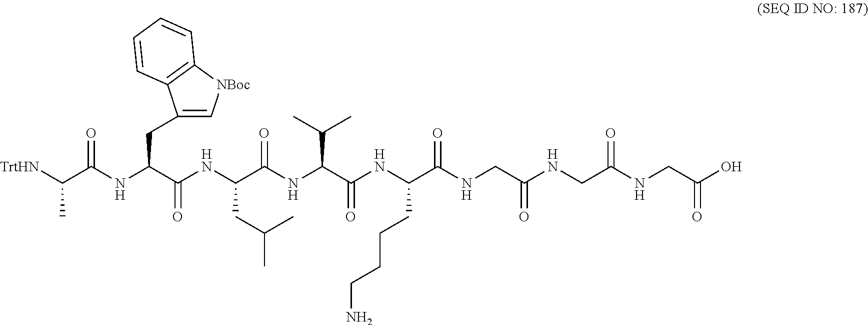

or a salt (e.g., a carboxylate salt) thereof.

In some embodiments, the protected variant of Fragment 5 has the structure

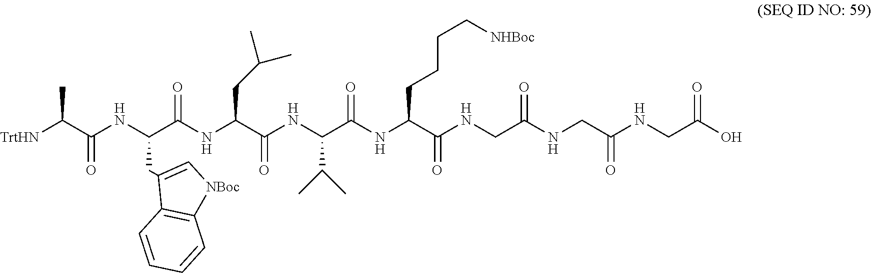

or a salt (e.g., a carboxylate salt) thereof.

### Fragment 6

Also provided herein is a peptide having a sequence of Gly-Gly-Gly-Gly-Ser-Gly-Gly-Gly-Gly-Ser-Gly-Gly-Gly-Gly-Ser-Lys (SEQ ID NO: 6) (“Fragment 6”). Certain aspects provide a protected variant of Fragment 6, wherein at least one of the Ser and Lys residues is protected with a protecting group. In addition, certain aspects provide a protected variant of Fragment 6, wherein the Lys residue at position 16 is protected with a side chain protecting group that can be selectively removed (i.e., is orthogonal to any other side chain or termini protecting group(s), such as, e.g., a 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), benzyloxycarbonyl (Cbz), or chlorobenzyloxycarbonyl(2-Cl—Z) protecting group) to enable selective modification of the &-amino group of the C-terminal Lys residue.

In some embodiments, the disclosure provides a protected variant of Fragment 6, wherein:
the side chain of the Ser residue at position 5, position 10, or position 15 is protected with a tert-butyl (tBu), O-trityl (OTrt), O-allyl (OAll), or pseudoproline (YPro) protecting group; and/or
the side chain of the Lys residue at position 16 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), 4-methyltrityl (Mtt), or benzyloxycarbonyl (Cbz) protecting group.

In some embodiments, the N-terminus of the Gly residue at position 1 is protected with a 9-fluorenylmethoxycarbonyl (Fmoc) protecting group.

In some embodiments, the disclosure provides a protected variant of Fragment 6, wherein:
the side chain of the Ser residue at position 5, position 10, or position 15 is protected with a tert-butyl (tBu) or O-trityl (OTrt) protecting group; and/or
the side chain of the Lys residue at position 16 is protected with a tert-butyloxycarbonyl (Boc), 1-(4,4-dimethyl-2,6-dioxocyclohex-1-ylidene)ethyl (Dde), or allyloxycarbonyl (Alloc) protecting group.

In some embodiments, the N-terminus of the Gly residue at position 1 is protected with a 9-fluorenylmethoxycarbonyl (Fmoc) protecting group.

In some embodiments, the disclosure provides a protected variant of Fragment 6, wherein:
the N-terminus of the Gly residue at position 1 is protected with a 9-fluorenylmethoxycarbonyl (Fmoc) protecting group;
the side chain of the Ser residue at position 5, position 10, or position 15 is protected with a tert-butyl (tBu), O-trityl (OTrt), or pseudoproline (YPro) protecting group; and
the side chain of the Lys residue at position 16 is protected with an allyloxycarbonyl (Alloc) protecting group.

In some embodiments, the disclosure provides a protected variant of Fragment 6, wherein:
the N-terminus of the Gly residue at position 1 is protected with a 9-fluorenylmethoxycarbonyl (Fmoc) protecting group;
the side chain of the Ser residue at position 5, position 10, or position 15 is protected with a tert-butyl (tBu), O-trityl (OTrt), O-allyl (OAll), or pseudoproline (YPro) protecting group; and
the side chain of the Lys residue at position 16 is protected with a 1-(4,4-dimethyl-2,6-dioxocyclohex-1-ylidene)ethyl (Dde) or 1-(4,4-dimethyl-2,6-dioxocyclohex-1-ylidene)-3-methylbutyl (ivDde) protecting group.

In some embodiments, the disclosure provides a protected variant of Fragment 6, wherein:
the N-terminus of the Gly residue at position 1 is protected with a 9-fluorenylmethoxycarbonyl (Fmoc) protecting group;
the side chain of the Ser residue at position 5, position 10, or position 15 is protected with a tert-butyl (tBu), O-trityl (OTrt), O-allyl (OAll), or pseudoproline (Pro) protecting group; and
the side chain of the Lys residue at position 16 is protected with a benzyloxycarbonyl (Cbz) or chlorobenzyloxycarbonyl(2-Cl—Z) protecting group.

In some embodiments, the disclosure provides a protected variant of Fragment 6, wherein:
the side chain of the Ser residue at position 5, position 10, or position 15 is protected with a p-bromobenzyloxycarbonyl (Br-Z), O-benzyl (OBn), or O-allyl (OAll) protecting group; and/or
the side chain of the Lys residue at position 16 is protected with a benzyloxycarbonyl (Cbz), 2-chlorobenzyloxycarbonyl(2-Cl—Z), or allyloxycarbonyl (Alloc) protecting group.

In some embodiments, the N-terminus of the Gly residue at position 1 is protected with a tert-butyloxycarbonyl (Boc) protecting group.

In some embodiments, the disclosure provides a protected variant of Fragment 6, wherein:
the side chain of the Ser residue at position 5, position 10, or position 15 is protected with an O-benzyl (OBn) protecting group; and/or
the side chain of the Lys residue at position 16 is protected with a benzyloxycarbonyl (Cbz) or 2-chlorobenzyloxycarbonyl(2-Cl—Z) protecting group.

In some embodiments, the N-terminus of the Gly residue at position 1 is protected with a tert-butyloxycarbonyl (Boc) protecting group.

In some embodiments, the disclosure provides a protected variant of Fragment 6, wherein:
the N-terminus of the Gly residue at position 1 is protected with a tert-butyloxycarbonyl (Boc) protecting group;
the side chain of the Ser residue at position 5, position 10, or position 15 is protected with a p-bromobenzyloxycarbonyl (Br-Z) or O-benzyl (OBn) protecting group; and
the side chain of the Lys residue at position 16 is protected with an allyloxycarbonyl (Alloc) protecting group.

In some embodiments, the disclosure provides a protected variant of Fragment 6, wherein:
the N-terminus of the Gly residue at position 1 is protected with a tert-butyloxycarbonyl (Boc) protecting group;
the side chain of the Ser residue at position 5, position 10, or position 15 is protected with a p-bromobenzyloxycarbonyl (Br-Z), O-benzyl (OBn), or O-allyl (OAll) protecting group; and
the side chain of the Lys residue at position 16 is protected with a 1-(4,4-dimethyl-2,6-dioxocyclohex-1-ylidene)ethyl (Dde) or 1-(4,4-dimethyl-2,6-dioxocyclohex-1-ylidene)-3-methylbutyl (ivDde) protecting group.

In some embodiments, the disclosure provides a protected variant of Fragment 6, wherein:
the N-terminus of the Gly residue at position 1 is protected with a tert-butyloxycarbonyl (Boc) protecting group;
the side chain of the Ser residue at position 5, position 10, or position 15 is protected with an O-allyl (OAll) protecting group; and
the side chain of the Lys residue at position 16 is protected with a benzyloxycarbonyl (Cbz) or chlorobenzyloxycarbonyl(2-Cl—Z) protecting group.

Certain aspects provide a protected variant of Fragment 6, wherein the Gly residue at position 1 is protected with an Fmoc protecting group at the N-terminus. Certain aspects describe a protected variant of Fragment 6, wherein the Ser residue at position 5 is protected as a t-butyl ether (tBu). Certain aspects describe a protected variant of Fragment 6, wherein the Ser residue at position 5 is protected as a pseudoproline moiety. Certain aspects provide a protected variant of Fragment 6, wherein the Ser residue at position 10 is protected as a t-butyl ether (tBu). Certain aspects provide a protected variant of Fragment 6, wherein the Ser residue at position 10 is protected as a pseudoproline moiety. Certain aspects provide a protected variant of Fragment 6, wherein the Ser residue at position 15 is protected as a t-butyl ether (tBu). Certain aspects provide a protected variant of Fragment 6, wherein the Ser residue at position 15 is protected as a pseudoproline moiety. Certain aspects provide a protected variant of Fragment 6, wherein the Lys residue at position 16 is protected as a N-ε-1-(4,4-dimethyl-2,6-dioxocyclohex-1-ylidene)-3-methylbutyl amine (ivDde).

Certain aspects provide a protected variant of Fragment 6, wherein each of the Ser residues at positions 5, 10, and 15 is protected as a pseudoproline moiety. Certain aspects provide a protected variant of Fragment 6, wherein each of the Ser residues at positions 5, 10, and 15 is protected as a t-butyl ether (tBu).

In some embodiments, the present disclosure provides a protected variant of Fragment 6, wherein:
the Ser residue at position 5 is protected as a t-butyl ether (tBu); and/or
the Ser residue at position 10 is protected as a t-butyl ether (tBu); and/or
the Ser residue at position 15 is protected as a t-butyl ether (tBu); and/or
the Lys residue at position 16 is protected as a N-ε-1-(4,4-dimethyl-2,6-dioxocyclohex-1-ylidene)-3-methylbutyl amine (ivDde).

In some embodiments, the present disclosure provides a protected variant of Fragment 6, wherein:
the Ser residue at position 5 is protected as a t-butyl ether (tBu);
the Ser residue at position 10 is protected as a t-butyl ether (tBu);
the Ser residue at position 15 is protected as a t-butyl ether (tBu); and
the Lys residue at position 16 is protected as a N-ε-1-(4,4-dimethyl-2,6-dioxocyclohex-1-ylidene)-3-methylbutyl amine (ivDde).

In some embodiments, the Gly residue at position 1 is protected with an Fmoc protecting group or a Trt protecting group at the N-terminus. In some embodiments, the Gly residue at position 1 is protected with an Fmoc protecting group at the N-terminus. In some embodiments, the Gly residue at position 1 is protected with a Trt protecting group at the N-terminus.

In some embodiments, the protected variant of Fragment 6 does not have a free C-terminal carboxyl group. In some embodiments, the protected variant of Fragment 6 has a protected C-terminus. In some embodiments, the C-terminus of the protected variant of Fragment 6 is amidated.

In some embodiments, the protected variant of Fragment 6 is Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Fragment 6 does not have a free C-terminal carboxyl group (SEQ ID NO: 63), or a salt (e.g., an acid-addition salt) thereof.

In some embodiments, the protected variant of Fragment 6 is Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Fragment 6 has a protected C-terminus (SEQ ID NO: 120), or a salt (e.g., an acid-addition salt) thereof. In some embodiments, the protected variant of Fragment 6 is Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 121) or a salt (e.g., an acid-addition salt) thereof.

In some embodiments, the protected variant of Fragment 6 is Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Fragment 6 has a protected N-terminus and does not have a free C-terminal carboxyl group (SEQ ID NO: 70). In some embodiments, the protected variant of Fragment 6 is Fmoc-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Fragment 6 does not have a free C-terminal carboxyl group (SEQ ID NO: 77). In some embodiments, the protected variant of Fragment 6 is Trt-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Fragment 6 does not have a free C-terminal carboxyl group (SEQ ID NO: 123).

In some embodiments, the protected variant of Fragment 6 is Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Fragment 6 has a protected N-terminus and a protected C-terminus (SEQ ID NO: 124). In some embodiments, the protected variant of Fragment 6 is Fmoc-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Fragment 6 has a protected C-terminus (SEQ ID NO: 125). In some embodiments, the protected variant of Fragment 6 is Trt-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Fragment 6 has a protected C-terminus (SEQ ID NO: 126).

In some embodiments, the protected variant of Fragment 6 is Gly-Gly-Gly-Gly-Ser(tBu)-Gly-Gly-Gly-Gly-Ser(tBu)-Gly-Gly-Gly-Gly-Ser(tBu)-Lys(ivDde)-NH₂, wherein the protected variant of Fragment 6 has a protected N-terminus (SEQ ID NO: 122). In some embodiments, the protected variant of Fragment 6 is Fmoc-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 127). In some embodiments, the protected variant of Fragment 6 is Trt-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 128).

In some embodiments, the present disclosure provides a protected variant of Fragment 6, wherein:
the Ser residue at position 5 is protected as a pseudoproline moiety; and/or
the Ser residue at position 10 is protected as a pseudoproline moiety; and/or
the Ser residue at position 15 is protected as a pseudoproline moiety; and/or
the Lys residue at position 16 is protected as a N-ε-1-(4,4-dimethyl-2,6-dioxocyclohex-1-ylidene)-3-methylbutyl amine (ivDde).

In some embodiments, the present disclosure provides a protected variant of Fragment 6, wherein:
the Ser residue at position 5 is protected as a pseudoproline moiety;
the Ser residue at position 10 is protected as a pseudoproline moiety;
the Ser residue at position 15 is protected as a pseudoproline moiety; and
the Lys residue at position 16 is protected as a N-ε-1-(4,4-dimethyl-2,6-dioxocyclohex-1-ylidene)-3-methylbutyl amine (ivDde).

In some embodiments, the Gly residue at position 1 is protected with an Fmoc protecting group or a Trt protecting group at the N-terminus. In some embodiments, the Gly residue at position 1 is protected with an Fmoc protecting group at the N-terminus. In some embodiments, the Gly residue at position 1 is protected with a Trt protecting group at the N-terminus.

In some embodiments, the Gly residue at position 1 is protected with an Fmoc protecting group or a Trt protecting group at the N-terminus. In some embodiments, the Gly residue at position 1 is protected with an Fmoc protecting group at the N-terminus. In some embodiments, the Gly residue at position 1 is protected with a Trt protecting group at the N-terminus.

In some embodiments, the protected variant of Fragment 6 does not have a free C-terminal carboxyl group. In some embodiments, the protected variant of Fragment 6 has a protected C-terminus. In some embodiments, the C-terminus of the protected variant of Fragment 6 is amidated.

In some embodiments, the protected variant of Fragment 6 is Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me,Me)Pro)-Lys(ivDde), wherein the protected variant of Fragment 6 does not have a free C-terminal carboxyl group (SEQ ID NO: 62), or a salt (e.g., an acid-addition salt) thereof.

In some embodiments, the protected variant of Fragment 6 is Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde), wherein the protected variant of Fragment 6 has a protected C-terminus (SEQ ID NO: 129), or a salt (e.g., an acid-addition salt) thereof. In some embodiments, the protected variant of Fragment 6 is Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130) or a salt (e.g., an acid-addition salt) thereof.

In some embodiments, the protected variant of Fragment 6 is Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me,Me)Pro)-Lys(ivDde), wherein the protected variant of Fragment 6 has a protected N-terminus and does not have a free C-terminal carboxyl group (SEQ ID NO: 69). In some embodiments, the protected variant of Fragment 6 is Fmoc-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde), wherein the protected variant of Fragment 6 does not have a free C-terminal carboxyl group (SEQ ID NO: 132). In some embodiments, the protected variant of Fragment 6 is Trt-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde), wherein the protected variant of Fragment 6 does not have a free C-terminal carboxyl group (SEQ ID NO: 78).

In some embodiments, the protected variant of Fragment 6 is Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me,Me)Pro)-Lys(ivDde), wherein the protected variant of Fragment 6 has a protected N-terminus and a protected C-terminus (SEQ ID NO: 133). In some embodiments, the protected variant of Fragment 6 is Fmoc-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde), wherein the protected variant of Fragment 6 has a protected C-terminus (SEQ ID NO: 134). In some embodiments, the protected variant of Fragment 6 is Trt-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde), wherein the protected variant of Fragment 6 has a protected C-terminus (SEQ ID NO: 135).

In some embodiments, the protected variant of Fragment 6 is Gly-Gly-Gly-Gly-Ser(Psi(Me, Me)Pro)-Gly-Gly-Gly-Gly-Ser(Psi(Me, Me)Pro)-Gly-Gly-Gly-Gly-Ser(Psi(Me, Me)Pro)-Lys(ivDde)-NH₂, wherein the protected variant of Fragment 6 has a protected N-terminus (SEQ ID NO: 131). In some embodiments, the protected variant of Fragment 6 is Fmoc-Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 136). In some embodiments, the protected variant of Fragment 6 is Trt-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 137).

Certain aspects provide a protected variant of Fragment 6, having a structure of:

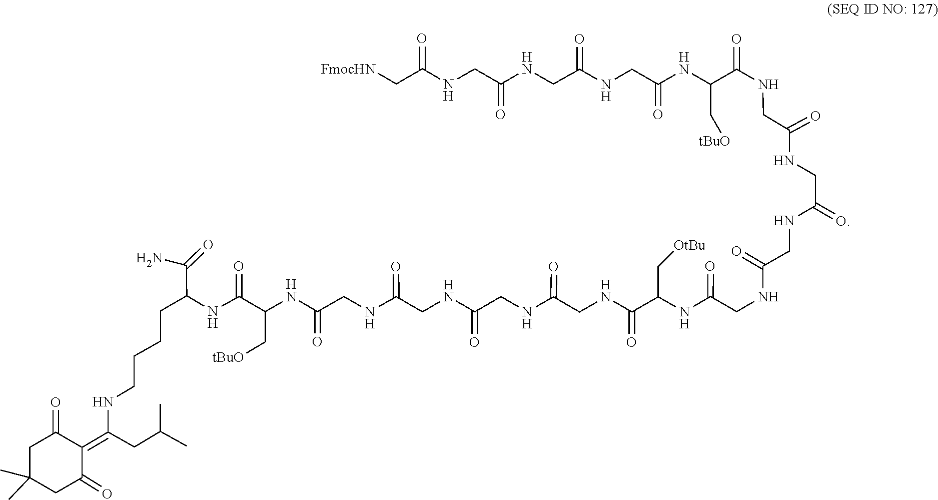

In some embodiments, the protected variant of Fragment 6 has the structure

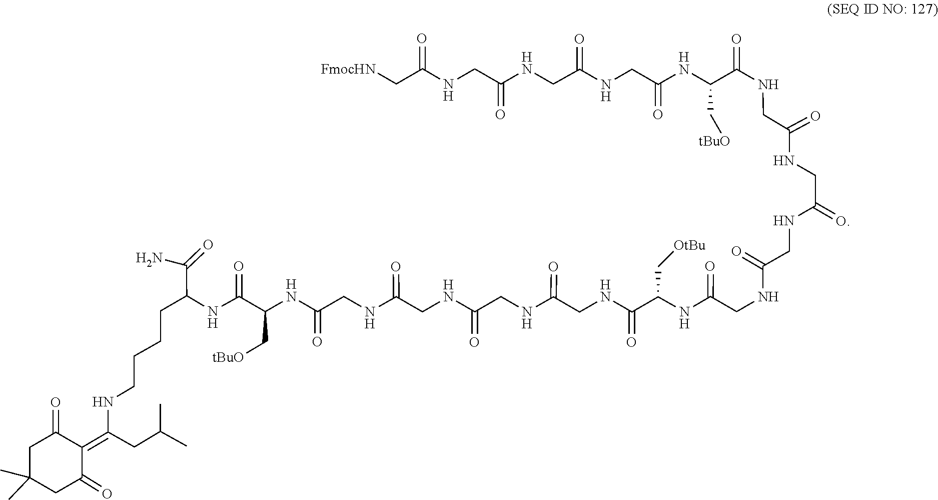

Certain aspects provide a protected variant of Fragment 6, having a structure of:

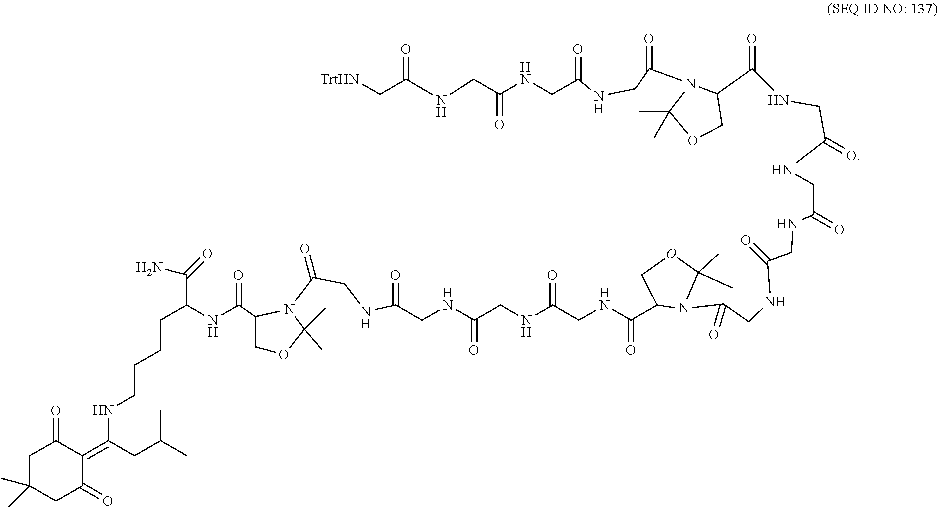

In some embodiments, the protected variant of Fragment 6 has the structure

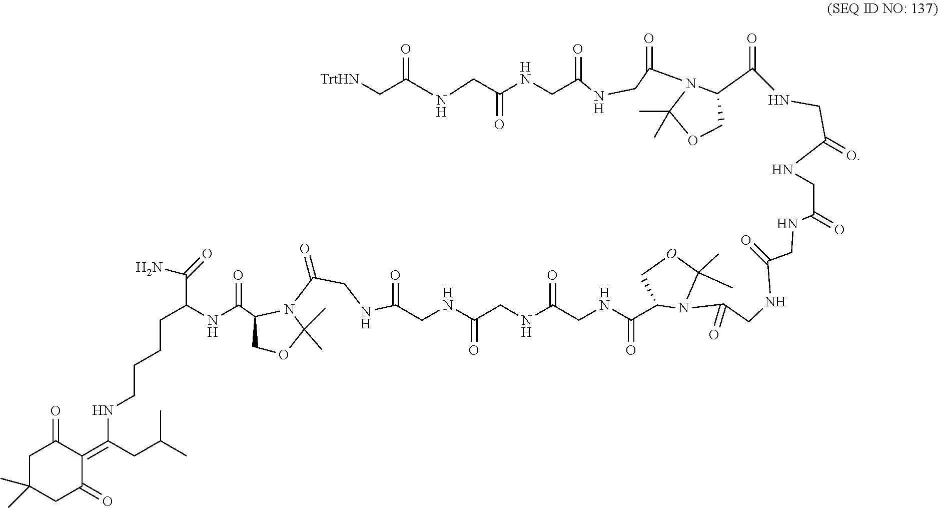

Certain aspects provide a protected variant of Fragment 6, having a structure of

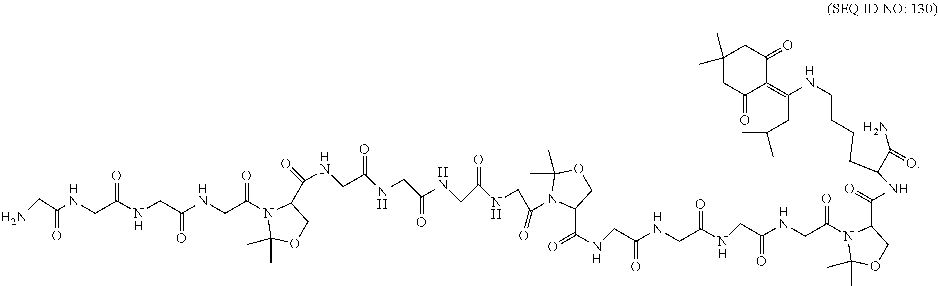 [SMILES: CC(C)CC(NCCCCC(NC(=O)C1COC(C)(C)N1C(=O)CNC(=O)CNC(=O)CNC(=O)CNC(=O)C1COC(C)(C)N1C(=O)CNC(=O)CNC(=O)CNC(=O)CNC(=O)C1COC(C)(C)N1C(=O)CNC(=O)CNC(=O)CNC(=O)CN)C(N)=O)=C1C(=O)CC(C)(C)CC1=O]

or a salt (e.g., an acid-addition salt) thereof.

In some embodiments, the protected variant of Fragment 6 has the structure

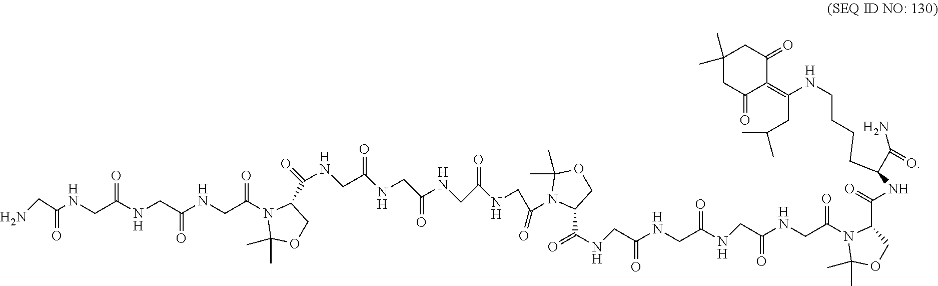 [SMILES: CC(C)CC(NCCCC[C@H](NC(=O)[C@@H]1COC(C)(C)N1C(=O)CNC(=O)CNC(=O)CNC(=O)CNC(=O)[C@H]1COC(C)(C)N1C(=O)CNC(=O)CNC(=O)CNC(=O)CNC(=O)[C@@H]1COC(C)(C)N1C(=O)CNC(=O)CNC(=O)CNC(=O)CN)C(N)=O)=C1C(=O)CC(C)(C)CC1=O]

or a salt (e.g., an acid-addition salt) thereof.

Certain aspects provide a conjugate of Fragment 6 or a protected variant thereof, wherein the Lys residue at position 16 is coupled to a solid support. In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing. In some embodiments, the conjugate further comprises a linker through which the peptide is attached to the solid support (e.g., the resin material). In some embodiments, the linker is a rink-amide linker or a Sieber-amide linker.

In some embodiments, the solid support is a swellable resin material, and the conjugate further comprises a linker through which the peptide is attached to the swellable resin material.

In some embodiments, the solid support is a swellable resin material, and the conjugate further comprises a linker through which the peptide is attached to the swellable resin material, wherein the linker is a rink-amide linker. In some embodiments, the solid support is a swellable resin material, and the conjugate further comprises a linker through which the peptide is attached to the swellable resin material, wherein the linker is a Sieber-amide linker.

In some embodiments, the solid support is a Rink amide, peptide amide linker (PAL), safety-catch amide linker (SCAL), or Sieber amide resin. In some embodiments, the N-terminus of the conjugate is protected with a 9-fluorenylmethoxycarbonyl (Fmoc) protecting group, and the solid support is a Rink amide, peptide amide linker (PAL), safety-catch amide linker (SCAL), or Sieber amide resin.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin, a 2-chlorodiphenylmethyl resin, a 4-(hydroxymethyl) phenoxyacetic acid (HMPA) resin, a 4-(4-hydroxymethyl-3-methoxyphenoxy) butyric acid (HMPB) resin, or a resin comprising a dialkoxybenzyl-type benzyl alcohol linker on a polystyrene support. In some embodiments, the N-terminus of the conjugate is protected with a 9-fluorenylmethoxycarbonyl (Fmoc) protecting group, and the solid support is a Wang (p-benzyloxybenzyl) resin, a 2-chlorodiphenylmethyl resin, a 4-(hydroxymethyl) phenoxyacetic acid (HMPA) resin, a 4-(4-hydroxymethyl-3-methoxyphenoxy) butyric acid (HMPB) resin, or a resin comprising a dialkoxybenzyl-type benzyl alcohol linker on a polystyrene support.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol-grafted polystyrene. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

Certain aspects provide a conjugate of a protected variant of Fragment 6, having a structure of

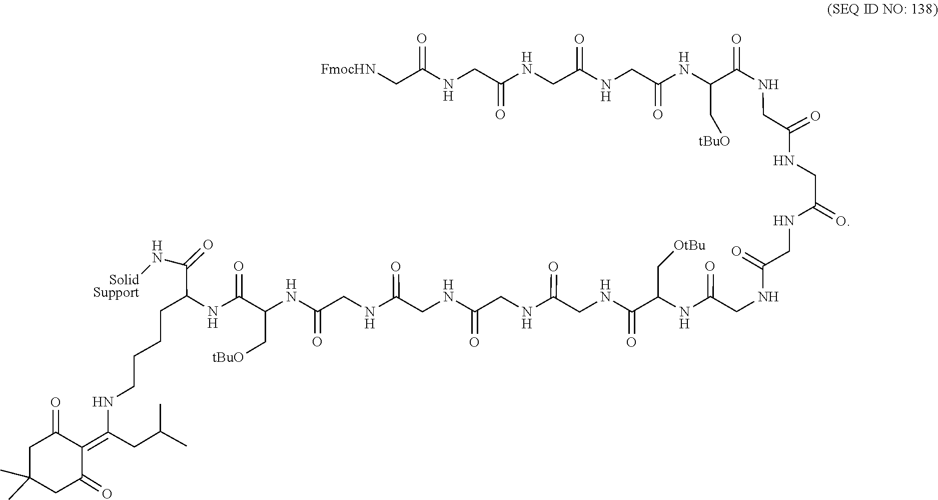

In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol-grafted polystyrene. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

In some embodiments, the conjugate of a protected variant of Fragment 6 has the structure

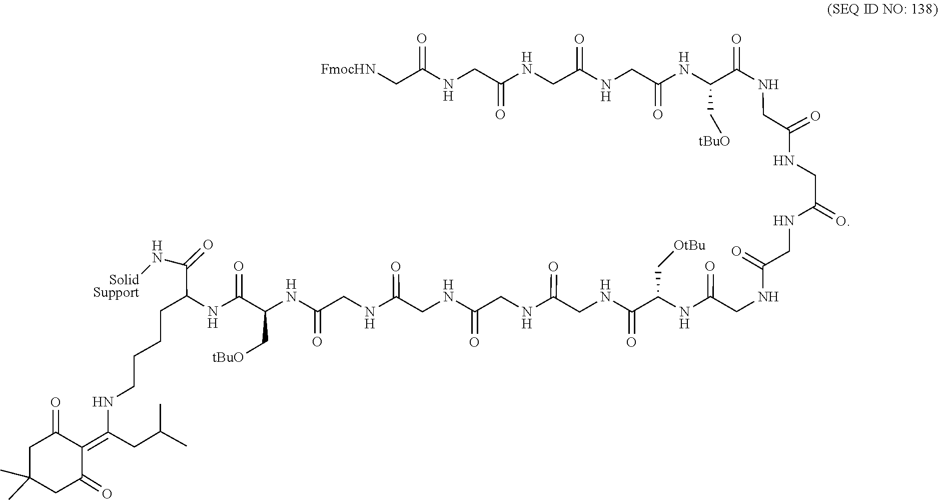

In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol-grafted polystyrene. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

### Peptide B

Also provided herein is a peptide having an amino acid sequence of Ala-Trp-Leu-Val-Lys-Gly-Gly-Gly-Gly-Gly-Gly-Gly-Ser-Gly-Gly-Gly-Gly-Ser-Gly-Gly-Gly-Gly-Ser-Lys (SEQ ID NO: 9) (“Peptide B”). Certain aspects provide a protected variant of Peptide B, wherein at least one of the Ala, Trp, Lys, and Ser residues is protected with a protecting group. In addition, certain aspects provide a protected variant of Peptide B, wherein the Lys residue at position 24 is protected with a side chain protecting group that can be selectively removed (i.e., is orthogonal to any other side chain or termini protecting group(s), such as, e.g., a 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), benzyloxycarbonyl (Cbz), or chlorobenzyloxycarbonyl(2-Cl—Z) protecting group) to enable selective modification of the &-amino group of the C-terminal Lys residue.

In some embodiments, the disclosure provides a protected variant of Peptide B, wherein:
the side chain of the Trp residue at position 2 is protected with a tert-butyloxycarbonyl (Boc) protecting group; and/or
the side chain of the Lys residue at position 5 or position 24 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), 4-methyltrityl (Mtt), or benzyloxycarbonyl (Cbz) protecting group; and/or
the side chain of the Ser residue at position 13, position 18, or position 23 is protected with a tert-butyl (tBu), O-trityl (OTrt), O-allyl (OAll), or pseudoproline (YPro) protecting group.

In some embodiments, the N-terminus of the Ala residue at position 1 is protected with a 9-fluorenylmethoxycarbonyl (Fmoc) protecting group.

In some embodiments, the disclosure provides a protected variant of Peptide B, wherein:
the side chain of the Trp residue at position 2 is protected with a tert-butyloxycarbonyl (Boc) protecting group; and/or
the side chain of the Lys residue at position 5 or position 24 is protected with a tert-butyloxycarbonyl (Boc), 1-(4,4-dimethyl-2,6-dioxocyclohex-1-ylidene)ethyl (Dde), or allyloxycarbonyl (Alloc) protecting group; and/or
the side chain of the Ser residue at position 13, position 18, or position 23 is protected with a tert-butyl (tBu) or O-trityl (OTrt) protecting group.

In some embodiments, the N-terminus of the Ala residue at position 1 is protected with a 9-fluorenylmethoxycarbonyl (Fmoc) protecting group.

In some embodiments, the disclosure provides a protected variant of Peptide B, wherein:
the N-terminus of the Ala residue at position 1 is protected with a 9-fluorenylmethoxycarbonyl (Fmoc) protecting group;
the side chain of the Trp residue at position 2 is protected with a tert-butyloxycarbonyl (Boc) protecting group;
the side chain of the Lys residue at position 5 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), trityl (Trt), 4-methyltrityl (Mtt), or benzyloxycarbonyl (Cbz) protecting group;
the side chain of the Ser residue at position 13, position 18, or position 23 is protected with a tert-butyl (tBu), O-trityl (OTrt), or pseudoproline (YPro) protecting group; and
the side chain of the Lys residue at position 24 is protected with an allyloxycarbonyl (Alloc) protecting group.

In some embodiments, the disclosure provides a protected variant of Peptide B, wherein:
the N-terminus of the Ala residue at position 1 is protected with a 9-fluorenylmethoxycarbonyl (Fmoc) protecting group;
the side chain of the Trp residue at position 2 is protected with a tert-butyloxycarbonyl (Boc) protecting group;
the side chain of the Lys residue at position 5 is protected with a tert-butyloxycarbonyl (Boc), allyloxycarbonyl (Alloc), trityl (Trt), 4-methyltrityl (Mtt), or benzyloxycarbonyl (Cbz) protecting group;
the side chain of the Ser residue at position 13, position 18, or position 23 is protected with a tert-butyl (tBu), O-trityl (OTrt), O-allyl (OAll), or pseudoproline (YPro) protecting group; and
the side chain of the Lys residue at position 24 is protected with a 1-(4,4-dimethyl-2,6-dioxocyclohex-1-ylidene)ethyl (Dde) or 1-(4,4-dimethyl-2,6-dioxocyclohex-1-ylidene)-3-methylbutyl (ivDde) protecting group. In some embodiments, the disclosure provides a protected variant of Peptide B, wherein:
the N-terminus of the Ala residue at position 1 is protected with a 9-fluorenylmethoxycarbonyl (Fmoc) protecting group;
the side chain of the Trp residue at position 2 is protected with a tert-butyloxycarbonyl (Boc) protecting group;
the side chain of the Lys residue at position 5 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), or 4-methyltrityl (Mtt) protecting group;
the side chain of the Ser residue at position 13, position 18, or position 23 is protected with a tert-butyl (tBu), O-trityl (OTrt), O-allyl (OAll), or pseudoproline (YPro) protecting group; and
the side chain of the Lys residue at position 24 is protected with a benzyloxycarbonyl (Cbz) or chlorobenzyloxycarbonyl(2-Cl—Z) protecting group.

In some embodiments, the disclosure provides a protected variant of Peptide B, wherein:
the side chain of the Trp residue at position 2 is protected with a formyl (For) protecting group; and/or
the side chain of the Lys residue at position 5 or position 24 is protected with a benzyloxycarbonyl (Cbz), 2-chlorobenzyloxycarbonyl(2-Cl—Z), or allyloxycarbonyl (Alloc) protecting group; and/or
the side chain of the Ser residue at position 13, position 18, or position 23 is protected with a p-bromobenzyloxycarbonyl (Br-Z), O-benzyl (OBn), or O-allyl (OAll) protecting group.

In some embodiments, the N-terminus of the Ala residue at position 1 is protected with a tert-butyloxycarbonyl (Boc) protecting group.

In some embodiments, the disclosure provides a protected variant of Peptide B, wherein:
the side chain of the Trp residue at position 2 is protected with a formyl (For) protecting group; and/or
the side chain of the Lys residue at position 5 or position 24 is protected with a benzyloxycarbonyl (Cbz) or 2-chlorobenzyloxycarbonyl(2-Cl—Z) protecting group; and/or
the side chain of the Ser residue at position 13, position 18, or position 23 is protected with an O-benzyl (OBn) protecting group.

In some embodiments, the N-terminus of the Ala residue at position 1 is protected with a tert-butyloxycarbonyl (Boc) protecting group.

In some embodiments, the disclosure provides a protected variant of Peptide B, wherein:
the N-terminus of the Ala residue at position 1 is protected with a tert-butyloxycarbonyl (Boc) protecting group;
the side chain of the Trp residue at position 2 is protected with a formyl (For) protecting group;
the side chain of the Lys residue at position 5 is protected with a benzyloxycarbonyl (Cbz) or 2-chlorobenzyloxycarbonyl(2-Cl—Z) protecting group;
the side chain of the Ser residue at position 13, position 18, or position 23 is protected with a p-bromobenzyloxycarbonyl (Br-Z) or O-benzyl (OBn) protecting group; and
the side chain of the Lys residue at position 24 is protected with an allyloxycarbonyl (Alloc) protecting group.

In some embodiments, the disclosure provides a protected variant of Peptide B, wherein:
the N-terminus of the Ala residue at position 1 is protected with a tert-butyloxycarbonyl (Boc) protecting group;
the side chain of the Trp residue at position 2 is protected with a formyl (For) protecting group;
the side chain of the Lys residue at position 5 is protected with a benzyloxycarbonyl (Cbz), 2-chlorobenzyloxycarbonyl(2-Cl—Z), or allyloxycarbonyl (Alloc) protecting group;
the side chain of the Ser residue at position 13, position 18, or position 23 is protected with a p-bromobenzyloxycarbonyl (Br-Z), O-benzyl (OBn), or O-allyl (OAll) protecting group; and
the side chain of the Lys residue at position 24 is protected with a 1-(4,4-dimethyl-2,6-dioxocyclohex-1-ylidene)ethyl (Dde) or 1-(4,4-dimethyl-2,6-dioxocyclohex-1-ylidene)-3-methylbutyl (ivDde) protecting group.

In some embodiments, the disclosure provides a protected variant of Peptide B, wherein:
the N-terminus of the Ala residue at position 1 is protected with a tert-butyloxycarbonyl (Boc) protecting group;
the side chain of the Trp residue at position 2 is protected with a formyl (For) protecting group;
the side chain of the Lys residue at position 5 is protected with an allyloxycarbonyl (Alloc) protecting group;
the side chain of the Ser residue at position 13, position 18, or position 23 is protected with an O-allyl
(OAll) protecting group; and
the side chain of the Lys residue at position 24 is protected with a benzyloxycarbonyl (Cbz) or chlorobenzyloxycarbonyl(2-Cl—Z) protecting group.

Certain aspects provide a protected variant of Peptide B, wherein the Trp residue at position 2 is as t-butyloxy carbonyl amide (Boc). Certain aspects describe a protected variant of Peptide B, wherein the Lys residue at position 5 is protected as t-butyloxy carbonyl amide (Boc). Certain aspects describe a protected variant of Peptide B, wherein the Ser residue at position 13 is protected as a t-butyl ether (tBu). Alternative aspects describe a protected variant of Peptide B, wherein the Ser residue at position 13 is protected with a pseudoproline moiety. Certain aspects provide a protected variant of Peptide B, wherein the Ser residue at position 18 is protected as a t-butyl ether (tBu). Alternative aspects provide a protected variant of Peptide B, wherein the Ser residue at position 18 is protected as a pseudoproline moiety. Certain aspects provide a protected variant of Peptide B, wherein the Ser residue at position 23 is protected as a t-butyl ether (tBu). Alternative aspects provide a protected variant of Peptide B, wherein the Ser residue at position 23 is protected as a pseudoproline moiety. Certain aspects provide a protected variant of Peptide B, wherein the Lys residue at position 24 is protected as a N-ε-1-(4,4-dimethyl-2,6-dioxocyclohex-1-ylidene)-3-methylbutyl amine (ivDde).

In some embodiments, the present disclosure provides a protected variant of Peptide B, wherein:
the Ala residue at position 1 is protected with an Fmoc protecting group or a Trt protecting group at the N-terminus; and/or
the Trp residue at position 2 is protected as t-butyloxy carbonyl amide (Boc); and/or
the Lys residue at position 5 is protected as t-butyloxy carbonyl amide (Boc); and/or
the Ser residue at position 13 is protected as a t-butyl ether (tBu) or a pseudoproline moiety; and/or
the Ser residue at position 18 is protected as a t-butyl ether (tBu) or a pseudoproline moiety; and/or
the Ser residue at position 23 is protected as a t-butyl ether (tBu) or a pseudoproline moiety; and/or
the Lys residue at position 24 is protected as a N-ε-1-(4,4-dimethyl-2,6-dioxocyclohex-1-ylidene)-3-methylbutyl amine (ivDde).

In some embodiments, the present disclosure provides a protected variant of Peptide B, wherein each of the Ser residues at positions 13, 18, and 23 is protected as a t-butyl ether (tBu) or each of the Ser residues at positions 13, 18, and 23 is protected as a pseudoproline moiety.

In some embodiments, the present disclosure provides a protected variant of Peptide B, wherein:
the Ala residue at position 1 is protected with an Fmoc protecting group at the N-terminus; and/or
the Trp residue at position 2 is protected as t-butyloxy carbonyl amide (Boc); and/or
the Lys residue at position 5 is protected as t-butyloxy carbonyl amide (Boc); and/or
the Ser residue at position 13 is protected as a t-butyl ether (tBu) or a pseudoproline moiety; and/or
the Ser residue at position 18 is protected as a t-butyl ether (tBu) or a pseudoproline moiety; and/or
the Ser residue at position 23 is protected as a t-butyl ether (tBu) or a pseudoproline moiety; and/or
the Lys residue at position 24 is protected as a N-ε-1-(4,4-dimethyl-2,6-dioxocyclohex-1-ylidene)-3-methylbutyl amine (ivDde).

In some embodiments, the present disclosure provides a protected variant of Peptide B, wherein each of the Ser residues at positions 13, 18, and 23 is protected as a t-butyl ether (tBu) or each of the Ser residues at positions 13, 18, and 23 is protected as a pseudoproline moiety.

In some embodiments, the present disclosure provides a protected variant of Peptide B, wherein:
the Ala residue at position 1 is protected with an Fmoc protecting group at the N-terminus; and/or
the Trp residue at position 2 is protected as t-butyloxy carbonyl amide (Boc); and/or
the Lys residue at position 5 is protected as t-butyloxy carbonyl amide (Boc); and/or
the Ser residue at position 13 is protected as a t-butyl ether (tBu); and/or
the Ser residue at position 18 is protected as a t-butyl ether (tBu); and/or
the Ser residue at position 23 is protected as a t-butyl ether (tBu); and/or
the Lys residue at position 24 is protected as a N-ε-1-(4,4-dimethyl-2,6-dioxocyclohex-1-ylidene)-3-methylbutyl amine (ivDde).

In some embodiments, the present disclosure provides a protected variant of Peptide B, wherein each of the Ser residues at positions 13, 18, and 23 is protected as a t-butyl ether (tBu).

In some embodiments, the present disclosure provides a protected variant of Peptide B, wherein:
the Ala residue at position 1 is protected with an Fmoc protecting group at the N-terminus; and/or
the Trp residue at position 2 is protected as t-butyloxy carbonyl amide (Boc); and/or
the Lys residue at position 5 is protected as t-butyloxy carbonyl amide (Boc); and/or
the Ser residue at position 13 is protected as a pseudoproline moiety; and/or
the Ser residue at position 18 is protected as a pseudoproline moiety; and/or
the Ser residue at position 23 is protected as a pseudoproline moiety; and/or
the Lys residue at position 24 is protected as a N-ε-1-(4,4-dimethyl-2,6-dioxocyclohex-1-ylidene)-3-methylbutyl amine (ivDde).

In some embodiments, the present disclosure provides a protected variant of Peptide B, wherein each of the Ser residues at positions 13, 18, and 23 is protected as a pseudoproline moiety.

In some embodiments, the present disclosure provides a protected variant of Peptide B, wherein:
the Ala residue at position 1 is protected with an Fmoc protecting group at the N-terminus;
the Trp residue at position 2 is protected as t-butyloxy carbonyl amide (Boc);
the Lys residue at position 5 is protected as t-butyloxy carbonyl amide (Boc);
the Ser residue at position 13 is protected as a t-butyl ether (tBu) or a pseudoproline moiety;
the Ser residue at position 18 is protected as a t-butyl ether (tBu) or a pseudoproline moiety;
the Ser residue at position 23 is protected as a t-butyl ether (tBu) or a pseudoproline moiety; and
the Lys residue at position 24 is protected as a N-ε-1-(4,4-dimethyl-2,6-dioxocyclohex-1-ylidene)-3-methylbutyl amine (ivDde).

In some embodiments, the present disclosure provides a protected variant of Peptide B, wherein each of the Ser residues at positions 13, 18, and 23 is protected as a t-butyl ether (tBu) or each of the Ser residues at positions 13, 18, and 23 is protected as a pseudoproline moiety.

In some embodiments, the present disclosure provides a protected variant of Peptide B, wherein: the Trp residue at position 2 is protected as t-butyloxy carbonyl amide (Boc);
the Lys residue at position 5 is protected as t-butyloxy carbonyl amide (Boc);
the Ser residue at position 13 is protected as a t-butyl ether (tBu);
the Ser residue at position 18 is protected as a t-butyl ether (tBu);
the Ser residue at position 23 is protected as a t-butyl ether (tBu); and
the Lys residue at position 24 is protected as a N-ε-1-(4,4-dimethyl-2,6-dioxocyclohex-1-ylidene)-3-methylbutyl amine (ivDde).

In some embodiments, the Ala residue at position 1 is protected with an Fmoc protecting group at the N-terminus.

In some embodiments, the present disclosure provides a protected variant of Peptide B, wherein:
the Trp residue at position 2 is protected as t-butyloxy carbonyl amide (Boc);
the Lys residue at position 5 is protected as t-butyloxy carbonyl amide (Boc);
the Ser residue at position 13 is protected as a pseudoproline moiety;
the Ser residue at position 18 is protected as a pseudoproline moiety;
the Ser residue at position 23 is protected as a pseudoproline moiety; and

the Lys residue at position 24 is protected as a N-ε-1-(4,4-dimethyl-2,6-dioxocyclohex-1-ylidene)-3-methylbutyl amine (ivDde).

In some embodiments, the Ala residue at position 1 is protected with an Fmoc protecting group at the N-terminus.

In some embodiments, the Ala residue at position 1 is protected with a Trt protecting group at the N-terminus.

In some embodiments, the protected variant of Peptide B does not have a free C-terminal carboxyl group. In some embodiments, the protected variant of Peptide B has a protected C-terminus. In some embodiments, the C-terminus of the protected variant of Peptide B is amidated.

In some embodiments, the protected variant of Peptide B is Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide B does not have a free C-terminal carboxyl group (SEQ ID NO: 64), or a salt (e.g., an acid-addition salt) thereof.

In some embodiments, the protected variant of Peptide B is Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide B has a protected C-terminus (SEQ ID NO: 139), or a salt (e.g., an acid-addition salt) thereof. In some embodiments, the protected variant of Peptide B is Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 140) or a salt (e.g., an acid-addition salt) thereof.

In some embodiments, the protected variant of Peptide B is Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide B has a protected N-terminus and does not have a free C-terminal carboxyl group (SEQ ID NO: 71). In some embodiments, the protected variant of Peptide B is Fmoc-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide B does not have a free C-terminal carboxyl group (SEQ ID NO: 79).

In some embodiments, the protected variant of Peptide B is Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide B has a protected N-terminus and a protected C-terminus (SEQ ID NO: 142). In some embodiments, the protected variant of Peptide B is Fmoc-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide B has a protected C-terminus (SEQ ID NO: 143).

In some embodiments, the protected variant of Peptide B is Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂, wherein the protected variant of Peptide B has a protected N-terminus (SEQ ID NO: 141). In some embodiments, the protected variant of Peptide B is Fmoc-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 144).

In some embodiments, the protected variant of Peptide B is Ala-Trp(Boc)-Leu-Val-Lys(Boc)-Gly-Gly-Gly-Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me,Me)Pro)-Lys(ivDde), wherein the protected variant of Peptide B does not have a free C-terminal carboxyl group (SEQ ID NO: 145), or a salt (e.g., an acid-addition salt) thereof.

In some embodiments, the protected variant of Peptide B is Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me, Me)Pro)-Gly-Gly-Gly-Gly-Ser(Psi(Me,Me)Pro)-Lys(ivDde), wherein the protected variant of Peptide B has a protected C-terminus (SEQ ID NO: 146), or a salt (e.g., an acid-addition salt) thereof. In some embodiments, the protected variant of Peptide B is Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 147) or a salt (e.g., an acid-addition salt) thereof.

In some embodiments, the protected variant of Peptide B is Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde), wherein the protected variant of Peptide B has a protected N-terminus and does not have a free C-terminal carboxyl group (SEQ ID NO: 72). In some embodiments, the protected variant of Peptide B is Fmoc-Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde), wherein the protected variant of Peptide B does not have a free C-terminal carboxyl group (SEQ ID NO: 80). In some embodiments, the protected variant of Peptide B is Trt-Ala-Trp(Boc)-Leu-Val-Lys(Boc)-Gly-Gly-Gly-Gly-Gly-Gly-Gly-Ser(Psi(Me, Me)Pro)-Gly-Gly-Gly-Gly-Ser(Psi(Me, Me)Pro)-Gly-Gly-Gly-Gly-Ser(Psi(Me, Me)Pro)-Lys(ivDde), wherein the protected variant of Peptide B does not have a free C-terminal carboxyl group (SEQ ID NO: 178).

In some embodiments, the protected variant of Peptide B is Ala-Trp(Boc)-Leu-Val-Lys(Boc)-Gly-Gly-Gly-Gly-Gly-Gly-Gly-Ser(Psi(Me, Me)Pro)-Gly-Gly-Gly-Gly-Ser(Psi(Me, Me)Pro)-Gly-Gly-Gly-Gly-Ser(Psi(Me,Me)Pro)-Lys(ivDde), wherein the protected variant of Peptide B has a protected N-terminus and a protected C-terminus (SEQ ID NO: 149). In some embodiments, the protected variant of Peptide B is Fmoc-Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde), wherein the protected variant of Peptide B has a protected C-terminus (SEQ ID NO: 150). In some embodiments, the protected variant of Peptide Bis Trt-Ala-Trp(Boc)-Leu-Val-Lys(Boc)-Gly-Gly-Gly-Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me,Me)Pro)-Lys(ivDde), wherein the protected variant of Peptide B has a protected C-terminus (SEQ ID NO: 172).

In some embodiments, the protected variant of Peptide B is Ala-Trp(Boc)-Leu-Val-Lys(Boc)-Gly-Gly-Gly-Gly-Gly-Gly-Gly-Ser(Psi(Me, Me)Pro)-Gly-Gly-Gly-Gly-Ser(Psi(Me, Me)Pro)-Gly-Gly-Gly-Gly-Ser(Psi(Me,Me)Pro)-Lys(ivDde)-NH₂, wherein the protected variant of Peptide B has a protected N-terminus (SEQ ID NO: 148). In some embodiments, the protected variant of Peptide B is Fmoc-Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 151). In some embodiments, the protected variant of Peptide B is Trt-Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 170).

Certain aspects provide a protected variant of Peptide B, having a structure of

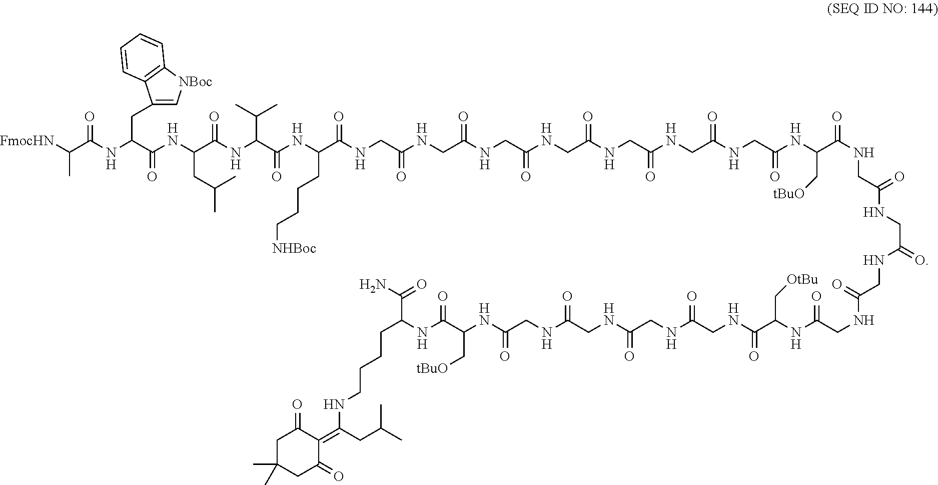

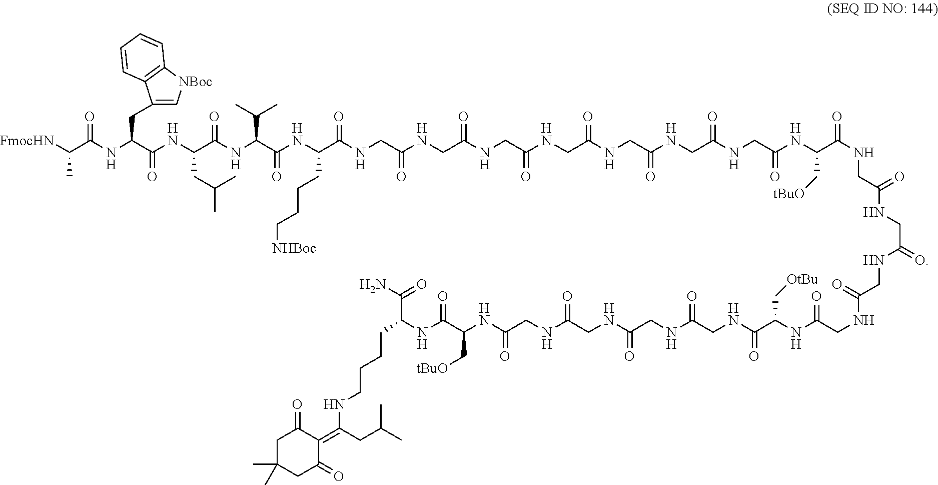

Certain aspects provide a protected variant of Peptide B, having a structure of

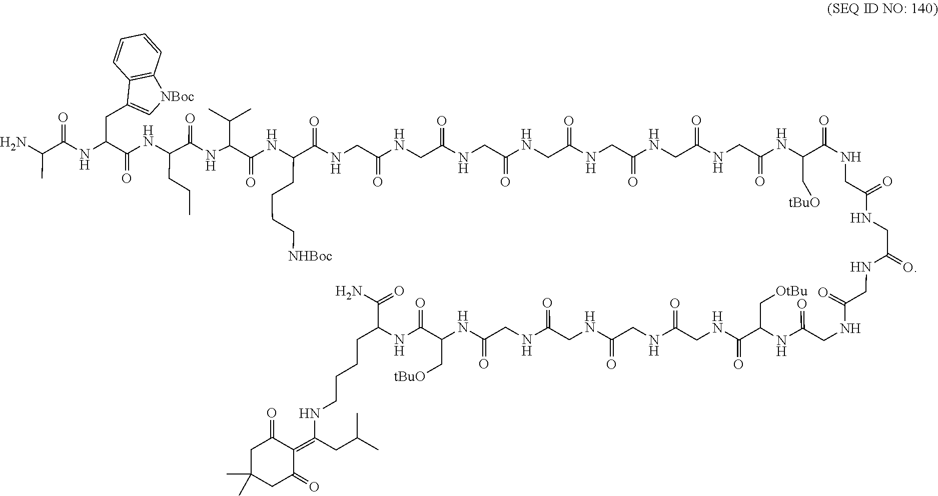 [SMILES: CCCC(NC(=O)C(Cc1cn(C(=O)OC(C)(C)C)c2ccccc12)NC(=O)C(C)N)C(=O)NC(C(=O)NC(CCCCNC(=O)OC(C)(C)C)C(=O)NCC(=O)NCC(=O)NCC(=O)NCC(=O)NCC(=O)NCC(=O)NCC(=O)NC(COC(C)(C)C)C(=O)NCC(=O)NCC(=O)NCC(=O)NCC(=O)NC(COC(C)(C)C)C(=O)NCC(=O)NCC(=O)NCC(=O)NCC(=O)NC(COC(C)(C)C)C(=O)NC(CCCCNC(CC(C)C)=C1C(=O)CC(C)(C)CC1=O)C(N)=O)C(C)C]

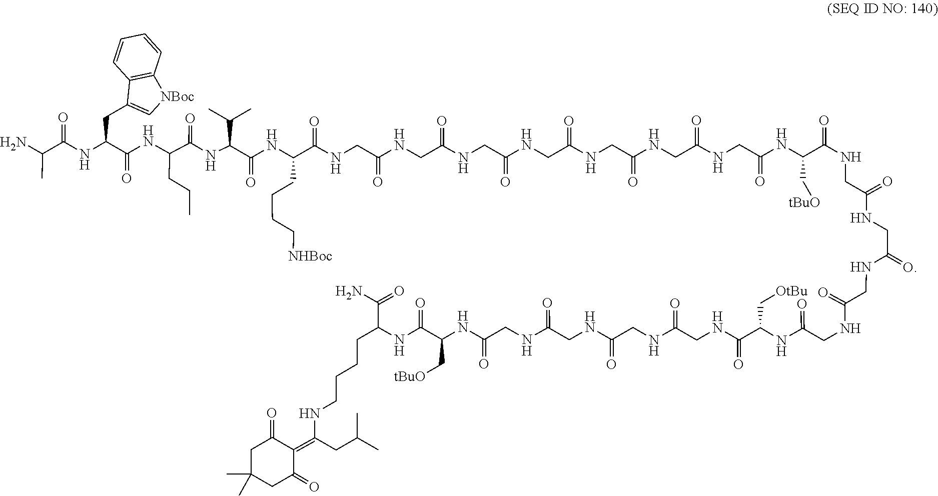 [SMILES: CCCC(NC(=O)[C@H](Cc1cn(C(=O)OC(C)(C)C)c2ccccc12)NC(=O)C(C)N)C(=O)N[C@H](C(=O)N[C@@H](CCCCNC(=O)OC(C)(C)C)C(=O)NCC(=O)NCC(=O)NCC(=O)NCC(=O)NCC(=O)NCC(=O)NCC(=O)N[C@@H](COC(C)(C)C)C(=O)NCC(=O)NCC(=O)NCC(=O)NCC(=O)N[C@@H](COC(C)(C)C)C(=O)NCC(=O)NCC(=O)NCC(=O)NCC(=O)N[C@@H](COC(C)(C)C)C(=O)NC(CCCCNC(CC(C)C)=C1C(=O)CC(C)(C)CC1=O)C(N)=O)C(C)C]

Certain aspects provide a protected variant of Peptide B, having a structure of

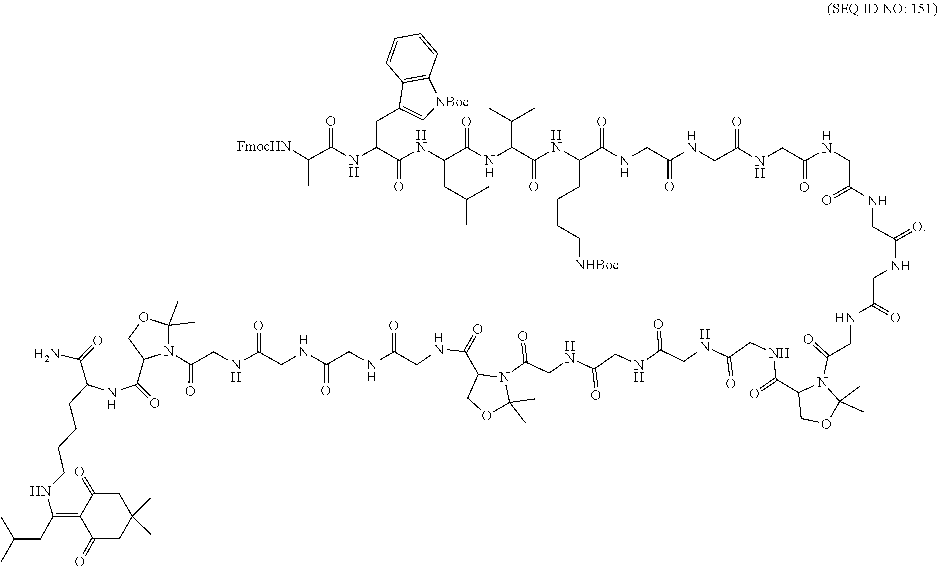

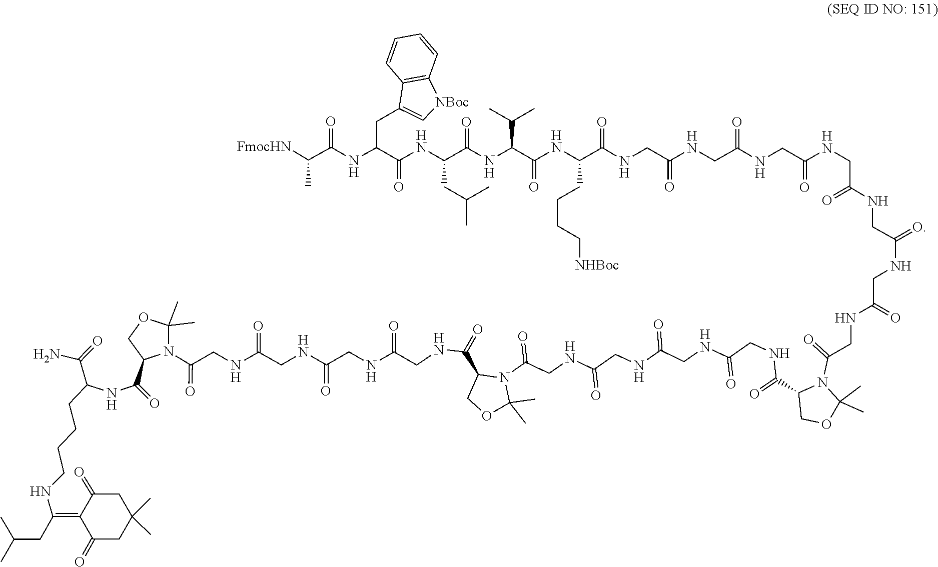

Certain aspects provide a protected variant of Peptide B, having a structure of

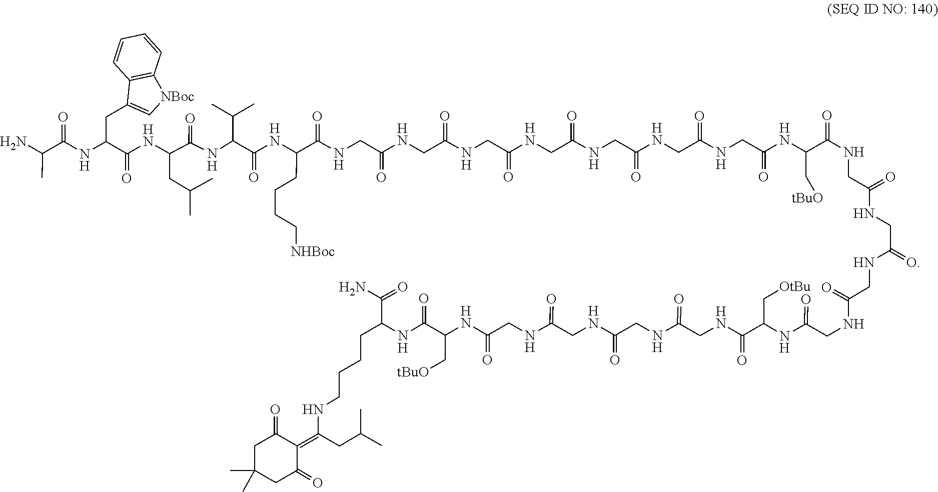 [SMILES: CC(C)CC(NCCCCC(NC(=O)C(COC(C)(C)C)NC(=O)CNC(=O)CNC(=O)CNC(=O)CNC(=O)C(COC(C)(C)C)NC(=O)CNC(=O)CNC(=O)CNC(=O)CNC(=O)C(COC(C)(C)C)NC(=O)CNC(=O)CNC(=O)CNC(=O)CNC(=O)CNC(=O)CNC(=O)CNC(=O)C(CCCCNC(=O)OC(C)(C)C)NC(=O)C(NC(=O)C(CC(C)C)NC(=O)C(Cc1cn(C(=O)OC(C)(C)C)c2ccccc12)NC(=O)C(C)N)C(C)C)C(N)=O)=C1C(=O)CC(C)(C)CC1=O]

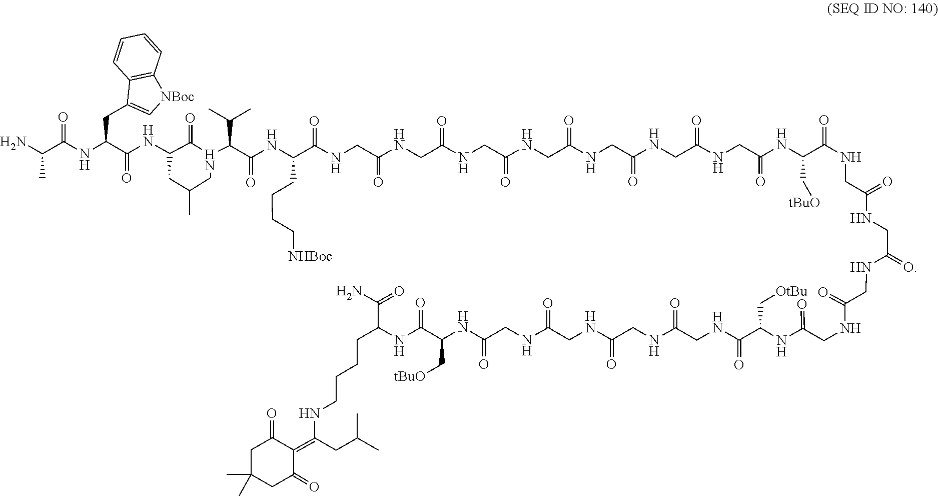

Certain aspects provide a protected variant of Peptide B, having a structure of

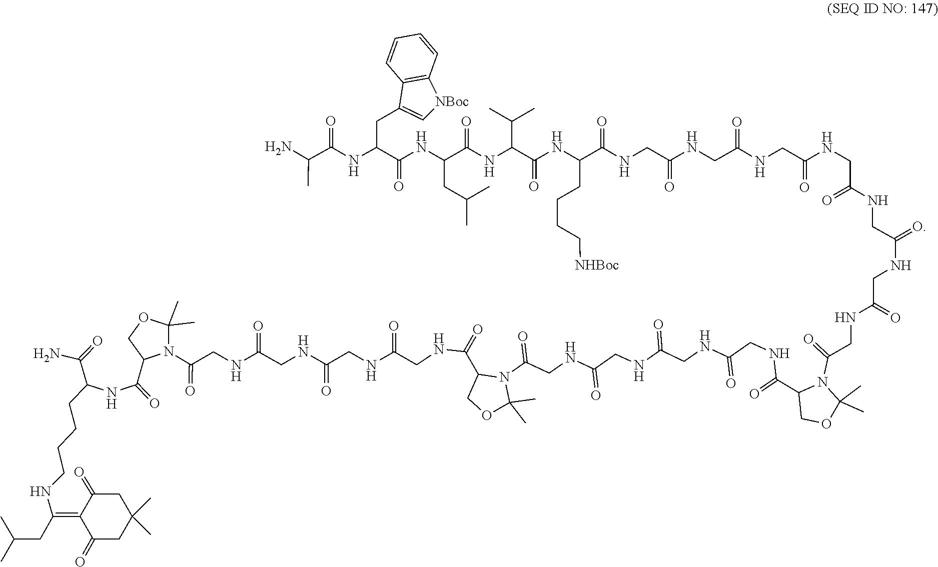 [SMILES: CC(C)CC(NCCCCC(NC(=O)C1COC(C)(C)N1C(=O)CNC(=O)CNC(=O)CNC(=O)CNC(=O)C1COC(C)(C)N1C(=O)CNC(=O)CNC(=O)CNC(=O)CNC(=O)C1COC(C)(C)N1C(=O)CNC(=O)CNC(=O)CNC(=O)CNC(=O)CNC(=O)CNC(=O)CNC(=O)C(CCCCNC(=O)OC(C)(C)C)NC(=O)C(NC(=O)C(CC(C)C)NC(=O)C(Cc1cn(C(=O)OC(C)(C)C)c2ccccc12)NC(=O)C(C)N)C(C)C)C(N)=O)=C1C(=O)CC(C)(C)CC1=O]

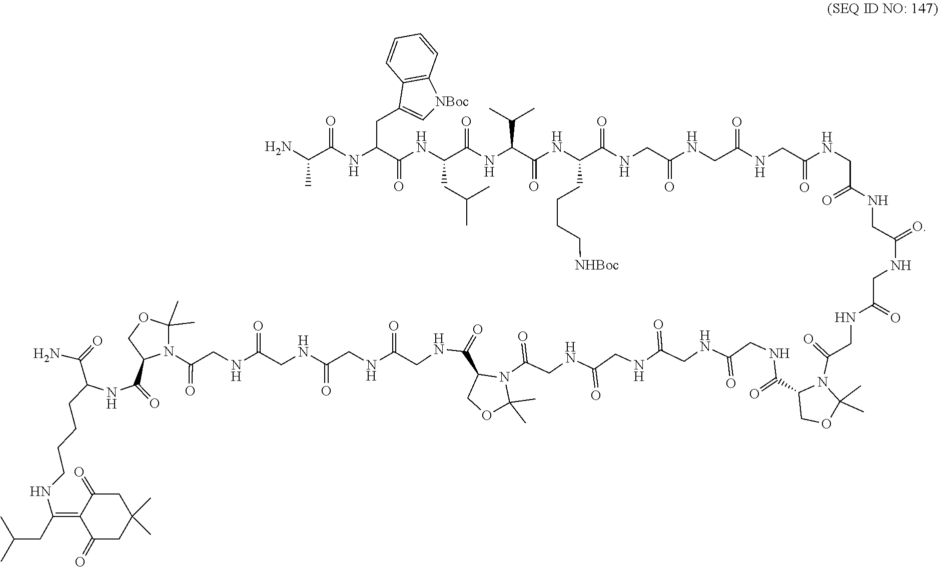 [SMILES: CC(C)CC(NCCCCC(NC(=O)[C@H]1COC(C)(C)N1C(=O)CNC(=O)CNC(=O)CNC(=O)CNC(=O)[C@@H]1COC(C)(C)N1C(=O)CNC(=O)CNC(=O)CNC(=O)CNC(=O)[C@H]1COC(C)(C)N1C(=O)CNC(=O)CNC(=O)CNC(=O)CNC(=O)CNC(=O)CNC(=O)CNC(=O)[C@H](CCCCNC(=O)OC(C)(C)C)NC(=O)[C@@H](NC(=O)[C@H](CC(C)C)NC(=O)C(Cc1cn(C(=O)OC(C)(C)C)c2ccccc12)NC(=O)[C@H](C)N)C(C)C)C(N)=O)=C1C(=O)CC(C)(C)CC1=O]

Certain aspects provide a protected variant of Peptide B, having a structure of

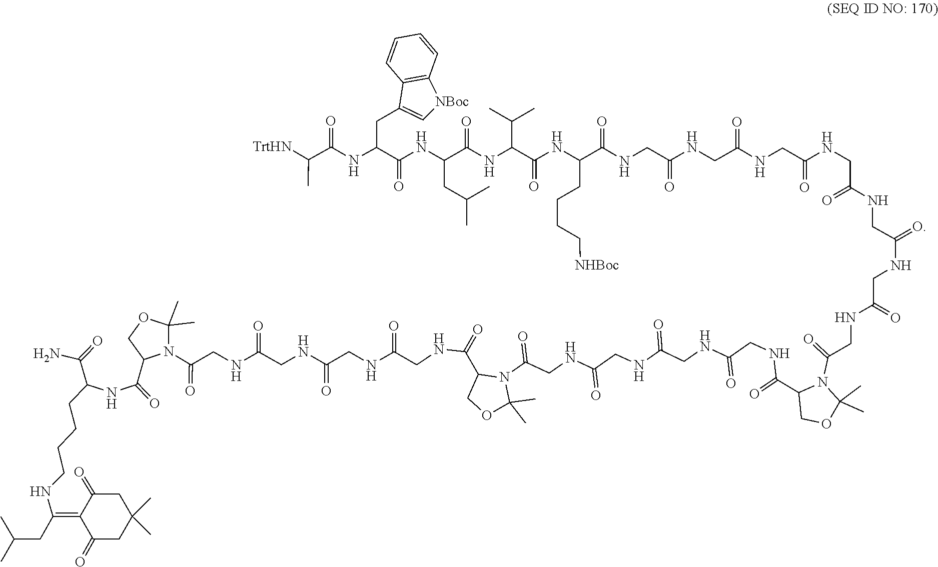

Certain aspects provide a conjugate of Peptide B or a protected variant thereof, wherein the Lys residue at position 24 is coupled to a solid support. Solid supports for solid phase synthesis are known in the art. In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing. In some embodiments, the conjugate further comprises a linker through which the peptide is attached to the solid support (e.g., the resin material). Linkers for solid support are known in the art. In some embodiments, the linker is a rink-amide linker or a Sieber-amide linker.

In some embodiments, the solid support is a swellable resin material, and the conjugate further comprises a linker through which the peptide is attached to the swellable resin material.

In some embodiments, the solid support is a swellable resin material, and the conjugate further comprises a linker through which the peptide is attached to the swellable resin material, wherein the linker is a rink-amide linker. In some embodiments, the solid support is a swellable resin material, and the conjugate further comprises a linker through which the peptide is attached to the swellable resin material, wherein the linker is a Sieber-amide linker.

In some embodiments, the solid support is a Rink amide, peptide amide linker (PAL), safety-catch amide linker (SCAL), or Sieber amide resin. In some embodiments, the N-terminus of the conjugate is protected with a 9-fluorenylmethoxycarbonyl (Fmoc) protecting group, and the solid support is a Rink amide, peptide amide linker (PAL), safety-catch amide linker (SCAL), or Sieber amide resin.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin, a 2-chlorodiphenylmethyl resin, a 4-(hydroxymethyl) phenoxyacetic acid (HMPA) resin, a 4-(4-hydroxymethyl-3-methoxyphenoxy) butyric acid (HMPB) resin, or a resin comprising a dialkoxybenzyl-type benzyl alcohol linker on a polystyrene support. In some embodiments, the N-terminus of the conjugate is protected with a 9-fluorenylmethoxycarbonyl (Fmoc) protecting group, and the solid support is a Wang (p-benzyloxybenzyl) resin, a 2-chlorodiphenylmethyl resin, a 4-(hydroxymethyl) phenoxyacetic acid (HMPA) resin, a 4-(4-hydroxymethyl-3-methoxyphenoxy) butyric acid (HMPB) resin, or a resin comprising a dialkoxybenzyl-type benzyl alcohol linker on a polystyrene support.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol-grafted polystyrene. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

In some embodiments, the solid support is a Merrifield (benzyl ester) resin, a hydroxymethyl polystyrene (HMP) resin, a benzhydrylamine (BHA) resin, a p-methylbenzhydrylamine (MBHA) resin, or a phenylacetamidomethyl (PAM) resin. In some embodiments, the N-terminus of the conjugate is protected with a tert-butyloxycarbonyl (Boc) protecting group, and the solid support is a Merrifield (benzyl ester) resin, a hydroxymethyl polystyrene (HMP) resin, a benzhydrylamine (BHA) resin, a p-methylbenzhydrylamine (MBHA) resin, or a phenylacetamidomethyl (PAM) resin.

In some embodiments, the solid support is a Merrifield (benzyl ester) resin. In some embodiments, the solid support is a hydroxymethyl polystyrene (HMP) resin. In some embodiments, the solid support is a benzhydrylamine (BHA) resin. In some embodiments, the solid support is a p-Methylbenzhydrylamine (MBHA) resin. In some embodiments, the solid support is a phenylacetamidomethyl (PAM) resin.

Certain aspects provide a conjugate of a protected variant of Peptide B, having a structure of

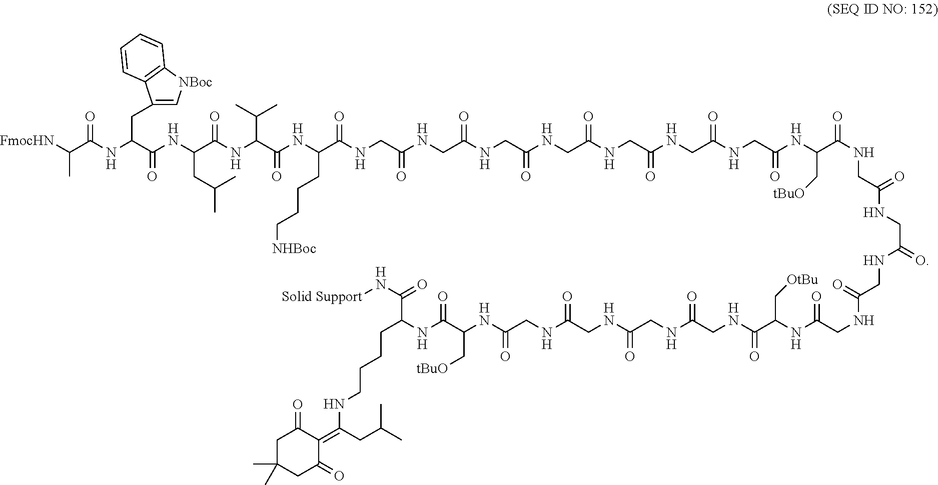

In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

In some embodiments, the conjugate of a protected variant of Peptide B has the structure

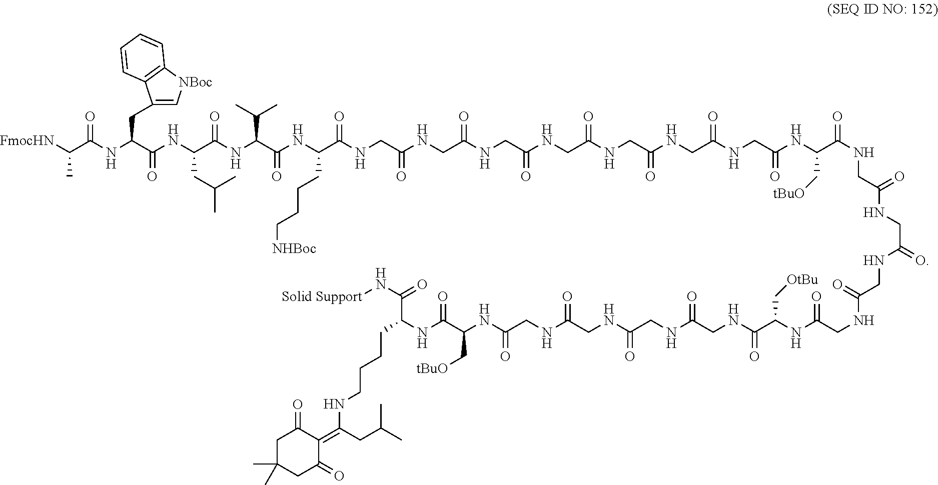

In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol-grafted polystyrene. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

### Peptide C

Also provided herein is a peptide having an amino acid sequence of Leu-Glu-Glu-Gln-Ala-Ala-Lys-Glu-Phe-Ile-Ala-Trp-Leu-Val-Lys-Gly-Gly-Gly-Gly-Gly-Gly-Gly-Ser-Gly-Gly-Gly-Gly-Ser-Gly-Gly-Gly-Gly-Ser-Lys (SEQ ID NO: 10) (“Peptide C”). Certain aspects provide a protected variant of Peptide C, wherein at least one of the Leu, Glu, Gln, Lys, Trp, and Ser residues is protected with a protecting group. In addition, certain aspects provide a protected variant of Peptide C, wherein the Lys residue at position 34 is protected with a side chain protecting group that can be selectively removed (i.e., is orthogonal to any other side chain or termini protecting group(s), such as, e.g., a 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), benzyloxycarbonyl (Cbz), or chlorobenzyloxycarbonyl(2-Cl—Z) protecting group) to enable selective modification of the s-amino group of the C-terminal Lys residue.

In some embodiments, the disclosure provides a protected variant of Peptide C, wherein:
the side chain of the Glu residue at position 2, position 3, or position 8 is protected with a tert-butyl ester (OtBu), O-2-phenylisopropyl ester (OPp), or allyl ester (OAll) protecting group; and/or
the side chain of the Gln residue at position 4 is protected with a trityl (Trt), benzyloxymethyl (Bom), xanthenyl (Xan), or p-methoxybenzyl (PMB) protecting group; and/or
the side chain of the Lys residue at position 7, position 15, or position 34 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), 4-methyltrityl (Mtt), or benzyloxycarbonyl (Cbz) protecting group; and/or
the side chain of the Trp residue at position 12 is protected with a tert-butyloxycarbonyl (Boc) protecting group; and/or
the side chain of the Ser residue at position 23, position 28, or position 33 is protected with a tert-butyl (tBu), O-trityl (OTrt), O-allyl (OAll), or pseudoproline (YPro) protecting group.

In some embodiments, the N-terminus of the Leu residue at position 1 is protected with a 9-fluorenylmethoxycarbonyl (Fmoc) protecting group.

In some embodiments, the disclosure provides a protected variant of Peptide C, wherein:
the side chain of the Glu residue at position 2, position 3, or position 8 is protected with a tert-butyl ester (OtBu) protecting group; and/or
the side chain of the Gln residue at position 4 is protected with a trityl (Trt) protecting group; and/or
the side chain of the Lys residue at position 7, position 15, or position 34 is protected with a tert-butyloxycarbonyl (Boc), 1-(4,4-dimethyl-2,6-dioxocyclohex-1-ylidene)ethyl (Dde), or allyloxycarbonyl (Alloc) protecting group; and/or
the side chain of the Trp residue at position 12 is protected with a tert-butyloxycarbonyl (Boc) protecting group; and/or
the side chain of the Ser residue at position 23, position 28, or position 33 is protected with a tert-butyl (tBu) or O-trityl (OTrt) protecting group.

In some embodiments, the N-terminus of the Leu residue at position 1 is protected with a 9-fluorenylmethoxycarbonyl (Fmoc) protecting group.

In some embodiments, the disclosure provides a protected variant of Peptide C, wherein:
the N-terminus of the Leu residue at position 1 is protected with a 9-fluorenylmethoxycarbonyl (Fmoc) protecting group;
the side chain of the Glu residue at position 2, position 3, or position 8 is protected with a tert-butyl ester (OtBu) or O-2-phenylisopropyl ester (OPp) protecting group;
the side chain of the Gln residue at position 4 is protected with a trityl (Trt), benzyloxymethyl (Bom), xanthenyl (Xan), or p-methoxybenzyl (PMB) protecting group;

the side chain of the Lys residue at position 7 or position 15 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), trityl (Trt), 4-methyltrityl (Mtt), or benzyloxycarbonyl (Cbz) protecting group;
the side chain of the Trp residue at position 12 is protected with a tert-butyloxycarbonyl (Boc) protecting group;
the side chain of the Ser residue at position 23, position 28, or position 33 is protected with a tert-butyl (tBu), O-trityl (OTrt), or pseudoproline (YPro) protecting group; and
the side chain of the Lys residue at position 34 is protected with an allyloxycarbonyl (Alloc) protecting group.

In some embodiments, the disclosure provides a protected variant of Peptide C, wherein:
the N-terminus of the Leu residue at position 1 is protected with a 9-fluorenylmethoxycarbonyl (Fmoc) protecting group;
the side chain of the Glu residue at position 2, position 3, or position 8 is protected with a tert-butyl ester (OtBu), O-2-phenylisopropyl ester (OPp), or allyl ester (OAll) protecting group;
the side chain of the Gln residue at position 4 is protected with a trityl (Trt), benzyloxymethyl (Bom), xanthenyl (Xan), or p-methoxybenzyl (PMB) protecting group;
the side chain of the Lys residue at position 7 or position 15 is protected with a tert-butyloxycarbonyl (Boc), allyloxycarbonyl (Alloc), trityl (Trt), 4-methyltrityl (Mtt), or benzyloxycarbonyl (Cbz) protecting group;
the side chain of the Trp residue at position 12 is protected with a tert-butyloxycarbonyl (Boc) protecting group;
the side chain of the Ser residue at position 23, position 28, or position 33 is protected with a tert-butyl (tBu), O-trityl (OTrt), O-allyl (OAll), or pseudoproline (YPro) protecting group; and

the side chain of the Lys residue at position 34 is protected with a 1-(4,4-dimethyl-2,6-dioxocyclohex-1-ylidene)ethyl (Dde) or 1-(4,4-dimethyl-2,6-dioxocyclohex-1-ylidene)-3-methylbutyl (ivDde) protecting group.

In some embodiments, the disclosure provides a protected variant of Peptide C, wherein:
the N-terminus of the Leu residue at position 1 is protected with a 9-fluorenylmethoxycarbonyl (Fmoc) protecting group;
the side chain of the Glu residue at position 2, position 3, or position 8 is protected with a tert-butyl ester (OtBu), O-2-phenylisopropyl ester (OPp), or allyl ester (OAll) protecting group;
the side chain of the Gln residue at position 4 is protected with a trityl (Trt), benzyloxymethyl (Bom), xanthenyl (Xan), or p-methoxybenzyl (PMB) protecting group;
the side chain of the Lys residue at position 7 or position 15 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), or 4-methyltrityl (Mtt) protecting group;
the side chain of the Trp residue at position 12 is protected with a tert-butyloxycarbonyl (Boc) protecting group;
the side chain of the Ser residue at position 23, position 28, or position 33 is protected with a tert-butyl (tBu), O-trityl (OTrt), O-allyl (OAll), or pseudoproline (YPro) protecting group; and
the side chain of the Lys residue at position 34 is protected with a benzyloxycarbonyl (Cbz) or chlorobenzyloxycarbonyl(2-Cl—Z) protecting group.

In some embodiments, the disclosure provides a protected variant of Peptide C, wherein:
the side chain of the Glu residue at position 2, position 3, or position 8 is protected with an allyl ester (OAll), benzyl ester (OBn), or cyclohexyl ester (OcHex) protecting group; and/or
the side chain of the Lys residue at position 7, position 15, or position 34 is protected with a benzyloxycarbonyl (Cbz), 2-chlorobenzyloxycarbonyl(2-Cl—Z), or allyloxycarbonyl (Alloc) protecting group; and/or
the side chain of the Trp residue at position 12 is protected with a formyl (For) protecting group; and/or
the side chain of the Ser residue at position 23, position 28, or position 33 is protected with a p-bromobenzyloxycarbonyl (Br—Z), O-benzyl (OBn), or O-allyl (OAll) protecting group.

In some embodiments, the N-terminus of the Leu residue at position 1 is protected with a tert-butyloxycarbonyl (Boc) protecting group.

In some embodiments, the disclosure provides a protected variant of Peptide C, wherein:
the side chain of the Glu residue at position 2, position 3, or position 8 is protected with a benzyl ester (OBn) or allyl ester (OAll) protecting group; and/or
the side chain of the Lys residue at position 7, position 15, or position 34 is protected with a benzyloxycarbonyl (Cbz) or 2-chlorobenzyloxycarbonyl(2-Cl—Z) protecting group; and/or
the side chain of the Trp residue at position 12 is protected with a formyl (For) protecting group; and/or
the side chain of the Ser residue at position 23, position 28, or position 33 is protected with an O-benzyl (OBn) protecting group.

In some embodiments, the N-terminus of the Leu residue at position 1 is protected with a tert-butyloxycarbonyl (Boc) protecting group.

In some embodiments, the disclosure provides a protected variant of Peptide C, wherein:
the N-terminus of the Leu residue at position 1 is protected with a tert-butyloxycarbonyl (Boc) protecting group;
the side chain of the Glu residue at position 2, position 3, or position 8 is protected with a benzyl ester (OBn) or cyclohexyl ester (OcHex) protecting group;
the side chain of the Lys residue at position 7 or position 15 is protected with a benzyloxycarbonyl (Cbz) or 2-chlorobenzyloxycarbonyl(2-Cl—Z) protecting group;
the side chain of the Trp residue at position 12 is protected with a formyl (For) protecting group;
the side chain of the Ser residue at position 23, position 28, or position 33 is protected with a p-bromobenzyloxycarbonyl (Br—Z) or O-benzyl (OBn) protecting group; and
the side chain of the Lys residue at position 34 is protected with an allyloxycarbonyl (Alloc) protecting group.

In some embodiments, the disclosure provides a protected variant of Peptide C, wherein:
the N-terminus of the Leu residue at position 1 is protected with a tert-butyloxycarbonyl (Boc) protecting group;
the side chain of the Glu residue at position 2, position 3, or position 8 is protected with an allyl ester (OAll), benzyl ester (OBn), or cyclohexyl ester (OcHex) protecting group;
the side chain of the Lys residue at position 7 or position 15 is protected with a benzyloxycarbonyl (Cbz), 2-chlorobenzyloxycarbonyl(2-Cl—Z), or allyloxycarbonyl (Alloc) protecting group;
the side chain of the Trp residue at position 12 is protected with a formyl (For) protecting group;
the side chain of the Ser residue at position 23, position 28, or position 33 is protected with a p-bromobenzyloxycarbonyl (Br—Z), O-benzyl (OBn), or O-allyl (OAll) protecting group; and
the side chain of the Lys residue at position 34 is protected with a 1-(4,4-dimethyl-2,6-dioxocyclohex-1-ylidene)ethyl (Dde) or 1-(4,4-dimethyl-2,6-dioxocyclohex-1-ylidene)-3-methylbutyl (ivDde) protecting group.

In some embodiments, the disclosure provides a protected variant of Peptide C, wherein:
the N-terminus of the Leu residue at position 1 is protected with a tert-butyloxycarbonyl (Boc) protecting group;
the side chain of the Glu residue at position 2, position 3, or position 8 is protected with an allyl ester (OAll) or cyclohexyl ester (OcHex) protecting group;
the side chain of the Lys residue at position 7 or position 15 is protected with an allyloxycarbonyl (Alloc) protecting group;
the side chain of the Trp residue at position 12 is protected with a formyl (For) protecting group;
the side chain of the Ser residue at position 23, position 28, or position 33 is protected with an O-allyl (OAll) protecting group; and
the side chain of the Lys residue at position 34 is protected with a benzyloxycarbonyl (Cbz) or chlorobenzyloxycarbonyl(2-Cl—Z) protecting group.

Certain aspects provide a protected variant of Peptide C, wherein the Leu residue at position 1 is protected with an Fmoc protecting group at the N-terminus. Certain aspects provide a protected variant of Peptide C, wherein the Glu residue at position 2 is protected as a t-butyl ester (OtBu). Certain aspects provide a protected variant of Peptide C, wherein the Glu residue at position 3 is protected as a t-butyl ester (OtBu). Certain aspects provide a protected variant of Peptide C, wherein the Gln residue at position 4 is protected as a triphenylmethyl amide (Trt). Certain aspects provide a protected variant of Peptide C, wherein the Lys residue at position 7 is protected as a t-butyloxy carbonyl amide (Boc). Certain aspects provide a protected variant of Peptide C, wherein the Glu residue at position 8 is protected as a t-butyl ester (OtBu). Certain aspects provide a protected variant of Peptide C, wherein the Trp residue at position 12 is protected as a t-butyloxy carbonyl amide (Boc). Certain aspects provide a protected variant of Peptide C, wherein the Lys residue at position 15 is protected as t-butyloxy carbonyl amide (Boc). Certain aspects provide a protected variant of Peptide C, wherein the Ser residue at position 23 is protected as a t-butyl ether (tBu). Certain aspects provide a protected variant of Peptide C, wherein the Ser residue at position 28 is protected as a t-butyl ether (tBu). Certain aspects provide a protected variant of Peptide C, wherein the Ser residue at position 33 is protected as a t-butyl ether (tBu). Certain aspects provide a protected variant of Peptide C, wherein the Lys residue at position 34 is protected as a N-ε-1-(4,4-dimethyl-2,6-dioxocyclohex-1-ylidene)-3-methylbutyl amine (ivDde).

In some embodiments, the present disclosure provides a protected variant of Peptide C, wherein:
the Leu residue at position 1 is protected with an Fmoc protecting group at the N-terminus; and/or
the Glu residue at position 2 is protected as a t-butyl ester (OtBu); and/or
the Glu residue at position 3 is protected as a t-butyl ester (OtBu); and/or
the Gln residue at position 4 is protected as a triphenylmethyl amide (Trt); and/or
the Lys residue at position 7 is protected as a t-butyloxy carbonyl amide (Boc); and/or
the Glu residue at position 8 is protected as a t-butyl ester (OtBu); and/or
the Trp residue at position 12 is protected as a t-butyloxy carbonyl amide (Boc); and/or
the Lys residue at position 15 is protected as t-butyloxy carbonyl amide (Boc); and/or
the Ser residue at position 23 is protected as a t-butyl ether (tBu); and/or
the Ser residue at position 28 is protected as a t-butyl ether (tBu); and/or
the Ser residue at position 33 is protected as a t-butyl ether (tBu); and/or
the Lys residue at position 34 is protected as a N-ε-1-(4,4-dimethyl-2,6-dioxocyclohex-1-ylidene)-3-methylbutyl amine (ivDde).

In some embodiments, the present disclosure provides a protected variant of Peptide C, wherein each of the Ser residues at positions 23, 28, and 33 is protected as a t-butyl ether (tBu).

In some embodiments, the present disclosure provides a protected variant of Peptide C, wherein: the Glu residue at position 2 is protected as a t-butyl ester (OtBu);
the Glu residue at position 3 is protected as a t-butyl ester (OtBu);
the Gln residue at position 4 is protected as a triphenylmethyl amide (Trt);
the Lys residue at position 7 is protected as a t-butyloxy carbonyl amide (Boc);
the Glu residue at position 8 is protected as a t-butyl ester (OtBu);
the Trp residue at position 12 is protected as a t-butyloxy carbonyl amide (Boc);
the Lys residue at position 15 is protected as t-butyloxy carbonyl amide (Boc);
the Ser residue at position 23 is protected as a t-butyl ether (tBu);
the Ser residue at position 28 is protected as a t-butyl ether (tBu);
the Ser residue at position 33 is protected as a t-butyl ether (tBu); and
the Lys residue at position 34 is protected as a N-ε-1-(4,4-dimethyl-2,6-dioxocyclohex-1-ylidene)-3-methylbutyl amine (ivDde).

In some embodiments, the Leu residue at position 1 is protected with an Fmoc protecting group at the N-terminus.

In some embodiments, the protected variant of Peptide C does not have a free C-terminal carboxyl group. In some embodiments, the protected variant of Peptide C has a protected C-terminus. In some embodiments, the C-terminus of the protected variant of Peptide C is amidated.

In some embodiments, the protected variant of Peptide C is Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide C does not have a free C-terminal carboxyl group (SEQ ID NO: 65), or a salt (e.g., an acid-addition salt) thereof.

In some embodiments, the protected variant of Peptide C is Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide C has a protected C-terminus (SEQ ID NO: 153), or a salt (e.g., an acid-addition salt) thereof. In some embodiments, the protected variant of Peptide C is Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 96) or a salt (e.g., an acid-addition salt) thereof.

In some embodiments, the protected variant of Peptide C is Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide C has a protected N-terminus and does not have a free C-terminal carboxyl group (SEQ ID NO: 73). In some embodiments, the protected variant of Peptide C is Fmoc-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide C does not have a free C-terminal carboxyl group (SEQ ID NO: 81).

In some embodiments, the protected variant of Peptide C is Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide C has a protected N-terminus and a protected C-terminus (SEQ ID NO: 154). In some embodiments, the protected variant of Peptide C is Fmoc-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide C has a protected C-terminus (SEQ ID NO: 155).

In some embodiments, the protected variant of Peptide C is Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂, wherein the protected variant of Peptide C has a protected N-terminus (SEQ ID NO: 97). In some embodiments, the protected variant of Peptide C is Fmoc-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 98).

Certain aspects provide a protected variant of Peptide C, having a structure of

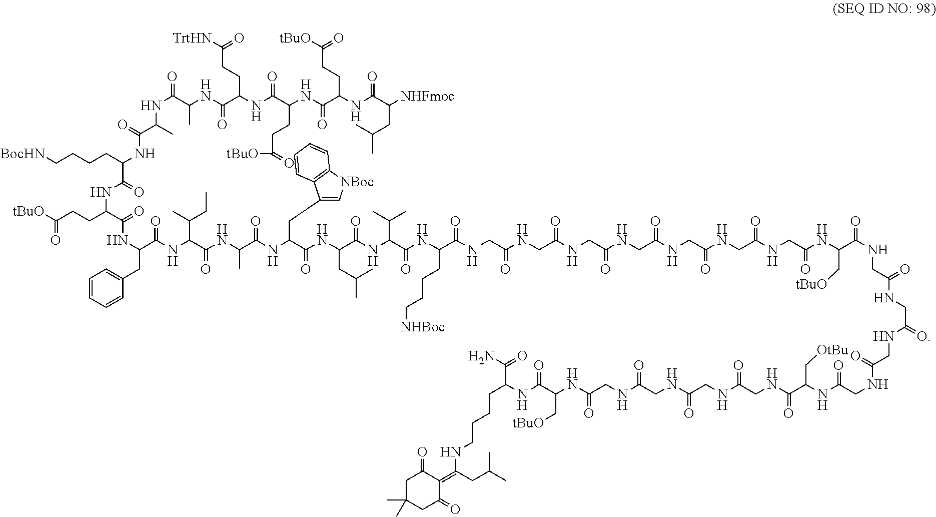

In some embodiments, the protected variant of Peptide C has the structure

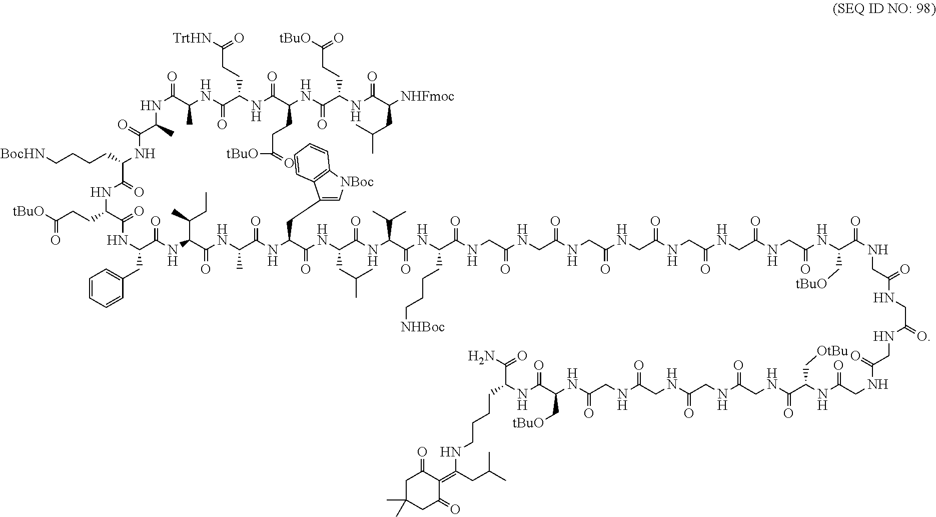

Certain aspects provide a conjugate of Peptide C or a protected variant thereof, wherein the Lys residue at position 34 is coupled to a solid support. Solid supports for solid phase synthesis are known in the art. In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing. In some embodiments, the conjugate further comprises a linker through which the peptide is attached to the solid support (e.g., the resin material). Linkers for solid support are known in the art. In some embodiments, the linker is a rink-amide linker or a Sieber-amide linker.

In some embodiments, the solid support is a swellable resin material, and the conjugate further comprises a linker through which the peptide is attached to the swellable resin material.

In some embodiments, the solid support is a swellable resin material, and the conjugate further comprises a linker through which the peptide is attached to the swellable resin material, wherein the linker is a rink-amide linker. In some embodiments, the solid support is a swellable resin material, and the conjugate further comprises a linker through which the peptide is attached to the swellable resin material, wherein the linker is a Sieber-amide linker.

In some embodiments, the solid support is a Rink amide, peptide amide linker (PAL), safety-catch amide linker (SCAL), or Sieber amide resin. In some embodiments, the N-terminus of the conjugate is protected with a 9-fluorenylmethoxycarbonyl (Fmoc) protecting group, and the solid support is a Rink amide, peptide amide linker (PAL), safety-catch amide linker (SCAL), or Sieber amide resin.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin, a 2-chlorodiphenylmethyl resin, a 4-(hydroxymethyl) phenoxyacetic acid (HMPA) resin, a 4-(4-hydroxymethyl-3-methoxyphenoxy) butyric acid (HMPB) resin, or a resin comprising a dialkoxybenzyl-type benzyl alcohol linker on a polystyrene support. In some embodiments, the N-terminus of the conjugate is protected with a 9-fluorenylmethoxycarbonyl (Fmoc) protecting group, and the solid support is a Wang (p-benzyloxybenzyl) resin, a 2-chlorodiphenylmethyl resin, a 4-(hydroxymethyl) phenoxyacetic acid (HMPA) resin, a 4-(4-hydroxymethyl-3-methoxyphenoxy) butyric acid (HMPB) resin, or a resin comprising a dialkoxybenzyl-type benzyl alcohol linker on a polystyrene support.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol-grafted polystyrene. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

In some embodiments, the solid support is a Merrifield (benzyl ester) resin, a hydroxymethyl polystyrene (HMP) resin, a benzhydrylamine (BHA) resin, a p-methylbenzhydrylamine (MBHA) resin, or a phenylacetamidomethyl (PAM) resin. In some embodiments, the N-terminus of the conjugate is protected with a tert-butyloxycarbonyl (Boc) protecting group, and the solid support is a Merrifield (benzyl ester) resin, a hydroxymethyl polystyrene (HMP) resin, a benzhydrylamine (BHA) resin, a p-methylbenzhydrylamine (MBHA) resin, or a phenylacetamidomethyl (PAM) resin.

In some embodiments, the solid support is a Merrifield (benzyl ester) resin. In some embodiments, the solid support is a hydroxymethyl polystyrene (HMP) resin. In some embodiments, the solid support is a benzhydrylamine (BHA) resin. In some embodiments, the solid support is a p-Methylbenzhydrylamine (MBHA) resin. In some embodiments, the solid support is a phenylacetamidomethyl (PAM) resin.

Certain aspects provide a conjugate of a protected variant of Peptide C, having a structure of

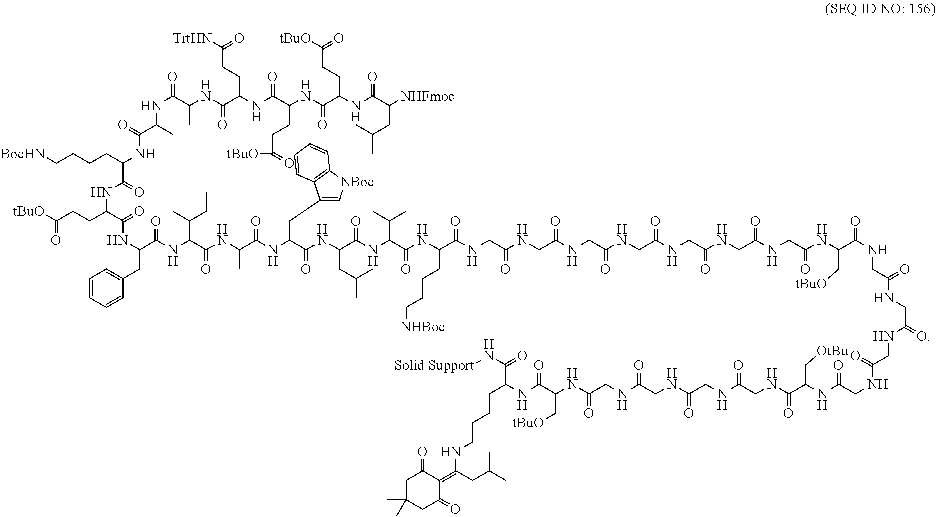

In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

In some embodiments, the conjugate of a protected variant of Peptide C has the structure

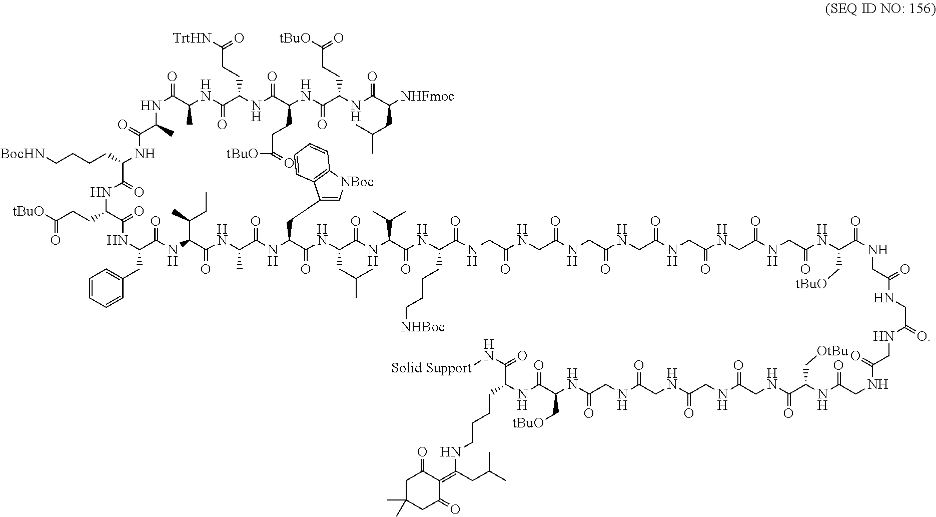

In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol-grafted polystyrene. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

### Peptide D

Also provided herein is a peptide having an amino acid sequence of His-Aib-Glu-Gly-Thr-Phe-Thr-Ser-Asp-Tyr-Ser-Ser-Tyr-Leu-Glu-Glu-Gln-Ala-Ala-Lys-Glu-Phe-Ile (SEQ ID NO: 15) (“Peptide D”). Certain aspects provide a protected variant of Peptide D, wherein at least one of the His, Glu, Thr, Ser, Asp, Tyr, Gln, and Lys residues is protected with a protecting group.

In some embodiments, the disclosure provides a protected variant of Peptide D, wherein:
the side chain of the His residue at position 1 is protected with a trityl (Trt), tert-butyloxycarbonyl (Boc), mesitylene-2-sulfonyl (Mts), monomethoxytrityl (Mmt), or 4-methyltrityl (Mtt) protecting group; and/or
the side chain of the Glu residue at position 3, position 15, position 16, or position 21 is protected with a tert-butyl ester (OtBu), O-2-phenylisopropyl ester (OPp), or allyl ester (OAll) protecting group; and/or
the side chain of the Thr residue at position 5 or position 7 is protected with a tert-butyl (tBu), O-trityl (OTrt), O-allyl (OAll), or pseudoproline (YPro) protecting group; and/or
the side chain of the Ser residue at position 8, position 11, or position 12 is protected with a tert-butyl (tBu), O-trityl (OTrt), O-allyl (OAll), or pseudoproline (YPro) protecting group; and/or
the side chain of the Asp residue at position 9 is protected with a tert-butyl ester (OtBu), O-2-phenylisopropyl ester (OPp), or allyl ester (OAll) protecting group; and/or
the side chain of the Tyr residue at position 10 or position 13 is protected with a tert-butyl (tBu), O-allyl (OAll), trityl (Trt), or 2-chlorotrityl (Clt) protecting group; and/or
the side chain of the Gln residue at position 17 is protected with a trityl (Trt), benzyloxymethyl (Bom), xanthenyl (Xan), or p-methoxybenzyl (PMB) protecting group; and/or
the side chain of the Lys residue at position 20 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), 4-methyltrityl (Mtt), or benzyloxycarbonyl (Cbz) protecting group.

In some embodiments, the N-terminus of the His residue at position 1 is protected with a 9-fluorenylmethoxycarbonyl (Fmoc) protecting group.

In some embodiments, the disclosure provides a protected variant of Peptide D, wherein:
the side chain of the His residue at position 1 is protected with a trityl (Trt) protecting group; and/or
the side chain of the Glu residue at position 3, position 15, position 16, or position 21 is protected with a tert-butyl ester (OtBu) protecting group; and/or
the side chain of the Thr residue at position 5 or position 7 is protected with a tert-butyl (tBu) or O-trityl (OTrt) protecting group; and/or
the side chain of the Ser residue at position 8, position 11, or position 12 is protected with a tert-butyl (tBu) or O-trityl (OTrt) protecting group; and/or
the side chain of the Asp residue at position 9 is protected with a tert-butyl ester (OtBu) protecting group; and/or
the side chain of the Tyr residue at position 10 or position 13 is protected with a tert-butyl (tBu) protecting group; and/or
the side chain of the Gln residue at position 17 is protected with a trityl (Trt) protecting group; and/or
the side chain of the Lys residue at position 20 is protected with a tert-butyloxycarbonyl (Boc), 1-(4,4-dimethyl-2,6-dioxocyclohex-1-ylidene)ethyl (Dde), or allyloxycarbonyl (Alloc) protecting group.

In some embodiments, the N-terminus of the His residue at position 1 is protected with a 9-fluorenylmethoxycarbonyl (Fmoc) protecting group.

In some embodiments, the disclosure provides a protected variant of Peptide D, wherein:
the side chain of the His residue at position 1 is protected with a 2,4-dinitrophenyl (Dnp), p-toluenesulfonyl (Tos), o-nitrobenzyl (ONb), or benzyloxymethyl (Bom) protecting group; and/or
the side chain of the Glu residue at position 3, position 15, position 16, or position 21 is protected with allyl ester (OAll), benzyl ester (OBn), or cyclohexyl ester (OcHex) protecting group; and/or
the side chain of the Thr residue at position 5 or position 7 is protected with a p-bromobenzyloxycarbonyl (Br—Z), O-benzyl (OBn), or O-allyl (OAll) protecting group; and/or
the side chain of the Ser residue at position 8, position 11, or position 12 is protected with a p-bromobenzyloxycarbonyl (Br—Z), O-benzyl (OBn), or O-allyl (OAll) protecting group; and/or
the side chain of the Asp residue at position 9 is protected with an allyl ester (OAll), benzyl ester (OBn), or cyclohexyl ester (OcHex) protecting group; and/or
the side chain of the Tyr residue at position 10 or position 13 is protected with an O-benzyl (OBn), O-allyl (OAll), O-(2-bromobenzyl), or 2,6-dichlorobenzyl (Dcb) protecting group; and/or
the side chain of the Lys residue at position 20 is protected with a benzyloxycarbonyl (Cbz), 2-chlorobenzyloxycarbonyl(2-Cl—Z), or allyloxycarbonyl (Alloc) protecting group.

In some embodiments, the N-terminus of the His residue at position 1 is protected with a tert-butyloxycarbonyl (Boc) protecting group.

In some embodiments, the disclosure provides a protected variant of Peptide D, wherein:
the side chain of the His residue at position 1 is protected with a p-toluenesulfonyl (Tos) protecting group; and/or
the side chain of the Glu residue at position 3, position 15, position 16, or position 21 is protected with a benzyl ester (OBn) or allyl ester (OAll) protecting group; and/or
the side chain of the Thr residue at position 5 or position 7 is protected with a O-benzyl (OBn) protecting group; and/or
the side chain of the Ser residue at position 8, position 11, or position 12 is protected with an O-benzyl (OBn) protecting group; and/or
the side chain of the Asp residue at position 9 is protected with a benzyl ester (OBn) or allyl ester (OAll) protecting group; and/or
the side chain of the Tyr residue at position 10 or position 13 is protected with an O-benzyl (OBn) protecting group; and/or
the side chain of the Lys residue at position 20 is protected with a benzyloxycarbonyl (Cbz) or 2-chlorobenzyloxycarbonyl(2-Cl—Z) protecting group.

In some embodiments, the N-terminus of the His residue at position 1 is protected with a tert-butyloxycarbonyl (Boc) protecting group.

Certain aspects provide a protected variant of Peptide D, wherein the His residue at position 1 is protected with a Boc protecting group at the N-terminus. Certain aspects provide a protected variant of Peptide D, wherein the His residue at position 1 is protected as a t-butyloxy carbonyl amide (Boc) at the His side chain. Certain aspects provide a protected variant of Peptide D, wherein the His residue at position 1 is protected with a Boc protecting group at the N-terminus, and the His residue at position 1 is protected as a t-butyloxy carbonyl amide (Boc) at the His side chain. Certain aspects provide a protected variant of Peptide D, wherein the His residue at position 1 is protected with a Boc protecting group at the N-terminus, and the side chain of the His residue at position 1 is protected with a Trt protecting group. Certain aspects provide a protected variant of Peptide D, wherein the Glu residue at position 3 is protected as a t-butyl ester (OtBu). Certain aspects provide a protected variant of Peptide D, wherein the Thr residue at position 5 is protected as a t-butyl ether (tBu). Certain aspects provide a protected variant of Peptide D, wherein the Thr residue at position 7 is protected as a t-butyl ether (tBu). Certain aspects provide a protected variant of Peptide D, wherein the Ser residue at position 8 is protected as a t-butyl ether (tBu). Certain aspects provide a protected variant of Peptide D, wherein the Asp residue at position 9 is protected as a t-butyl ester (OtBu). Certain aspects provide a protected variant of Peptide D, wherein the Tyr residue at position 10 is protected as a t-butyl ether (tBu). Certain aspects provide a protected variant of Peptide D, wherein the Ser residue at position 11 is protected as a t-butyl ether (tBu). Certain aspects provide a protected variant of Peptide D, wherein the Ser residue at position 12 is protected as a pseudoproline moiety. Certain aspects provide a protected variant of Peptide D, wherein the Tyr residue at position 13 is protected as a t-butyl ether (tBu). Certain aspects provide a protected variant of Peptide D, wherein the Glu residue at position 15 is protected as a t-butyl ester (OtBu). Certain aspects provide a protected variant of Peptide D, wherein the Glu residue at position 16 is protected as a t-butyl ester (OtBu). Certain aspects provide a protected variant of Peptide D, wherein the Gln residue at position 17 is protected as a triphenylmethyl amide (Trt). Certain aspects provide a protected variant of Peptide D, wherein the Lys residue at position 20 is protected as a t-butyloxy carbonyl amide (Boc). Certain aspects provide a protected variant of Peptide D, wherein the Glu residue at position 21 is protected as a t-butyl ester (OtBu).

In some embodiments, the present disclosure provides a protected variant of Peptide D, wherein each of the Ser residues at positions 8 and 11 is protected as a t-butyl ether (tBu). In some embodiments, the present disclosure provides a protected variant of Peptide D, wherein each of the Ser residues at positions 8 and 11 is protected as a t-butyl ether (tBu) and the Ser residue at position 12 is protected as a pseudoproline moiety.

In some embodiments, the present disclosure provides a protected variant of Peptide D, wherein each of the Glu residues at positions 3, 15, 16, and 21 is protected as a t-butyl ester (OtBu).

In some embodiments, the present disclosure provides a protected variant of Peptide D, wherein: the His residue at position 1 is protected with a Boc protecting group at the N-terminus; and/or
the His residue at position 1 is protected as a t-butyloxy carbonyl amide (Boc) at the His side chain; and/or
the Glu residue at position 3 is protected as a t-butyl ester (OtBu); and/or
the Thr residue at position 5 is protected as a t-butyl ether (tBu); and/or
the Thr residue at position 7 is protected as a t-butyl ether (tBu); and/or
the Ser residue at position 8 is protected as a t-butyl ether (tBu); and/or
the Asp residue at position 9 is protected as a t-butyl ester (OtBu); and/or
the Tyr residue at position 10 is protected as a t-butyl ether (tBu); and/or
the Ser residue at position 11 is protected as a t-butyl ether (tBu); and/or
the Ser residue at position 12 is protected as a pseudoproline moiety; and/or
the Tyr residue at position 13 is protected as a t-butyl ether (tBu); and/or
the Glu residue at position 15 is protected as a t-butyl ester (OtBu); and/or
the Glu residue at position 16 is protected as a t-butyl ester (OtBu); and/or
the Gln residue at position 17 is protected as a triphenylmethyl amide (Trt); and/or
the Lys residue at position 20 is protected as a t-butyloxy carbonyl amide (Boc); and/or
the Glu residue at position 21 is protected as a t-butyl ester (OtBu).

In alternative embodiments, the side chain of the His residue at position 1 is protected with a trityl (Trt) protecting group.

In some embodiments, the present disclosure provides a protected variant of Peptide D, wherein:
the His residue at position 1 is protected as a t-butyloxy carbonyl amide (Boc) at the His side chain;
the Glu residue at position 3 is protected as a t-butyl ester (OtBu);
the Thr residue at position 5 is protected as a t-butyl ether (tBu);
the Thr residue at position 7 is protected as a t-butyl ether (tBu);
the Ser residue at position 8 is protected as a t-butyl ether (tBu);
the Asp residue at position 9 is protected as a t-butyl ester (OtBu);
the Tyr residue at position 10 is protected as a t-butyl ether (tBu);
the Ser residue at position 11 is protected as a t-butyl ether (tBu);
the Ser residue at position 12 is protected as a pseudoproline moiety;
the Tyr residue at position 13 is protected as a t-butyl ether (tBu);
the Glu residue at position 15 is protected as a t-butyl ester (OtBu);
the Glu residue at position 16 is protected as a t-butyl ester (OtBu);
the Gln residue at position 17 is protected as a triphenylmethyl amide (Trt);
the Lys residue at position 20 is protected as a t-butyloxy carbonyl amide (Boc); and
the Glu residue at position 21 is protected as a t-butyl ester (OtBu).

In some embodiments, the His residue at position 1 is protected with a Boc protecting group at the N-terminus. In alternative embodiments, the side chain of the His residue at position 1 is protected with a trityl (Trt) protecting group.

In some embodiments, the protected variant of Peptide D is His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 180) or a salt (e.g., a carboxylate salt) thereof.

In some embodiments, the protected variant of Peptide D is His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-Leu-Glu(OtBu)-Glu(OtBu)-Gln(Trt)-Ala-Ala-Lys(Boc)-Glu(OtBu)-Phe-Ile-OH, wherein the protected variant of Peptide D has a protected N-terminus (SEQ ID NO: 181), or a salt (e.g., a carboxylate salt) thereof. In some embodiments, the protected variant of Peptide D is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 168) or a salt (e.g., a carboxylate salt) thereof.

In some embodiments, the protected variant of Peptide D is His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 22) or a salt (e.g., a carboxylate salt) thereof.

In some embodiments, the protected variant of Peptide D is His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH, wherein the protected variant of Peptide D has a protected N-terminus (SEQ ID NO: 27), or a salt (e.g., a carboxylate salt) thereof. In some embodiments, the protected variant of Peptide D is Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me, Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 157) or a salt (e.g., a carboxylate salt) thereof. In some embodiments, the protected variant of Peptide D is Fmoc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 158) or a salt (e.g., a carboxylate salt) thereof. In some embodiments, the protected variant of Peptide D is in the form of a carboxylate salt (e.g., a sodium or triethylammonium salt).

In some embodiments, the protected variant of Peptide D is His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 175) or a salt (e.g., a carboxylate salt) thereof.

In some embodiments, the protected variant of Peptide D is His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH, wherein the protected variant of Peptide D has a protected N-terminus (SEQ ID NO: 176), or a salt (e.g., a carboxylate salt) thereof. In some embodiments, the protected variant of Peptide D is Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 169) or a salt (e.g., a carboxylate salt) thereof. In some embodiments, the protected variant of Peptide D is Fmoc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 177) or a salt (e.g., a carboxylate salt) thereof. In some embodiments, the protected variant of Peptide D is in the form of a carboxylate salt (e.g., a sodium or triethylammonium salt).

Certain aspects provide a protected variant of Peptide D, having a structure of

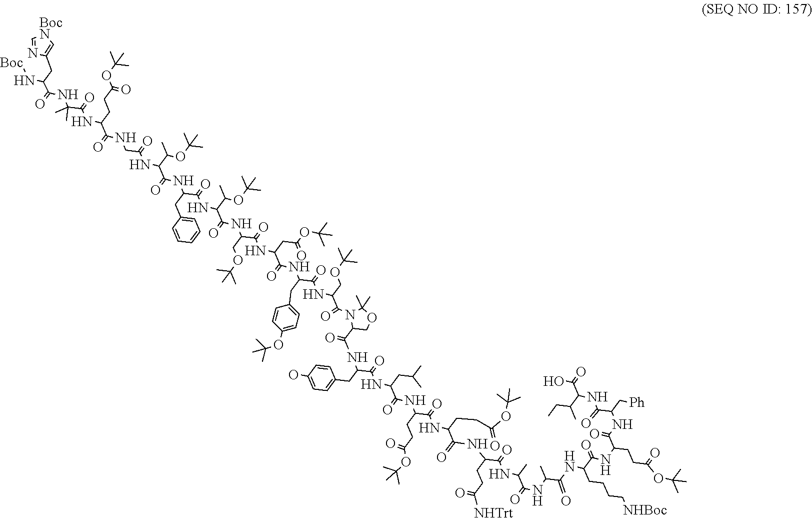

or a salt (e.g., a carboxylate salt) thereof.

In some embodiments, the protected variant of Peptide D has the structure

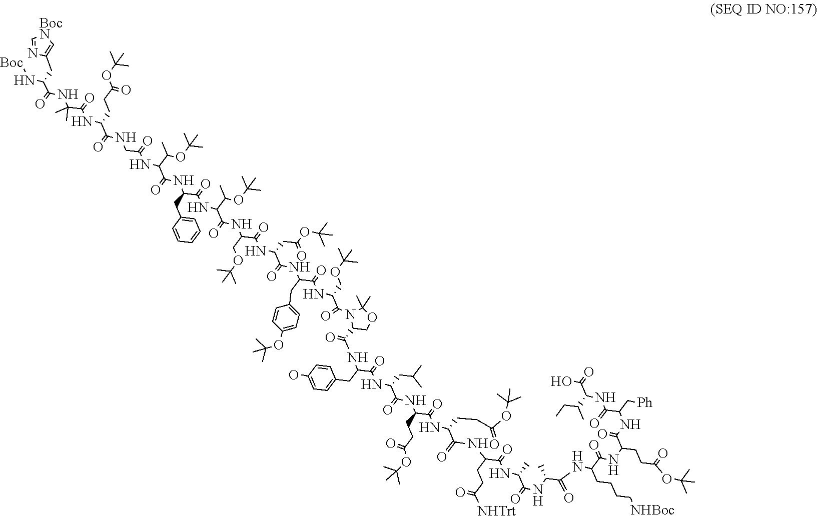

or a salt (e.g., a carboxylate salt) thereof.

Certain aspects provide a protected variant of Peptide D, having a structure of

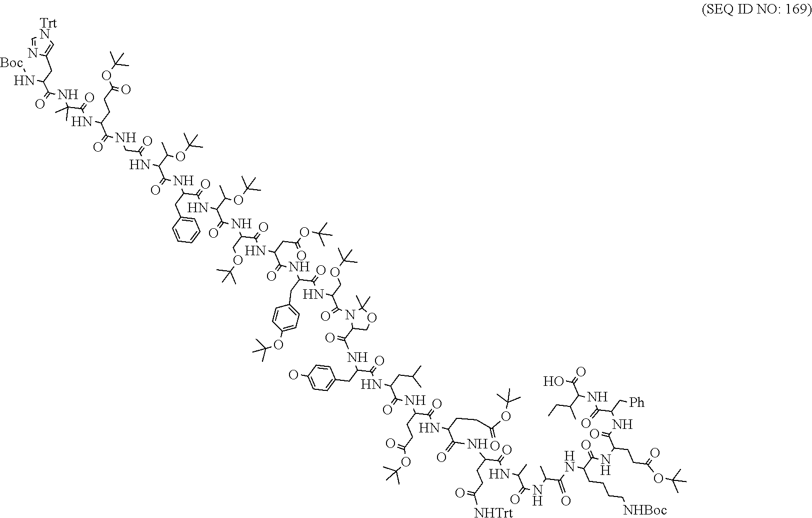

or a salt (e.g., a carboxylate salt) thereof.

### Peptide E

Also provided herein is a peptide having an amino acid sequence of Trp-Leu-Val-Lys-Gly-Gly-Gly-Gly-Gly-Gly-Gly-Ser-Gly-Gly-Gly-Gly-Ser-Gly-Gly-Gly-Gly-Ser-Lys (SEQ ID NO: 8) (“Peptide E”). Certain aspects provide a protected variant of Peptide E, wherein at least one of the Trp, Lys, and Ser residues is protected with a protecting group. In addition, certain aspects provide a protected variant of Peptide E, wherein the Lys residue at position 23 is protected with a side chain protecting group that can be selectively removed (i.e., is orthogonal to any other side chain or termini protecting group(s), such as, e.g., a 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), benzyloxycarbonyl (Cbz), or chlorobenzyloxycarbonyl(2-Cl—Z) protecting group) to enable selective modification of the &-amino group of the C-terminal Lys residue.

In some embodiments, the disclosure provides a protected variant of Peptide E, wherein:
the side chain of the Trp residue at position 1 is protected with a tert-butyloxycarbonyl (Boc) protecting group; and/or
the side chain of the Lys residue at position 4 or position 23 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), 4-methyltrityl (Mtt), or benzyloxycarbonyl (Cbz) protecting group; and/or
the side chain of the Ser residue at position 12, position 17, or position 22 is protected with a tert-butyl (tBu), O-trityl (OTrt), O-allyl (OAll), or pseudoproline (YPro) protecting group.

In some embodiments, the N-terminus of the Trp residue at position 1 is protected with a 9-fluorenylmethoxycarbonyl (Fmoc) protecting group.

In some embodiments, the disclosure provides a protected variant of Peptide E, wherein:
the side chain of the Trp residue at position 1 is protected with a tert-butyloxycarbonyl (Boc) protecting group; and/or
the side chain of the Lys residue at position 4 or position 23 is protected with a tert-butyloxycarbonyl (Boc), 1-(4,4-dimethyl-2,6-dioxocyclohex-1-ylidene)ethyl (Dde), or allyloxycarbonyl (Alloc) protecting group; and/or the side chain of the Ser residue at position 12, position 17, or position 22 is protected with a tert-butyl (tBu) or O-trityl (OTrt) protecting group.

In some embodiments, the N-terminus of the Trp residue at position 1 is protected with a 9-fluorenylmethoxycarbonyl (Fmoc) protecting group.

In some embodiments, the disclosure provides a protected variant of Peptide E, wherein:
the N-terminus of the Trp residue at position 1 is protected with a 9-fluorenylmethoxycarbonyl (Fmoc) protecting group;
the side chain of the Trp residue at position 1 is protected with a tert-butyloxycarbonyl (Boc) protecting group;
the side chain of the Lys residue at position 4 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), trityl (Trt), 4-methyltrityl (Mtt), or benzyloxycarbonyl (Cbz) protecting group;
the side chain of the Ser residue at position 12, position 17, or position 22 is protected with a tert-butyl (tBu), O-trityl (OTrt), or pseudoproline (Pro) protecting group; and
the side chain of the Lys residue at position 23 is protected with an allyloxycarbonyl (Alloc) protecting group.

In some embodiments, the disclosure provides a protected variant of Peptide E, wherein:

the N-terminus of the Trp residue at position 1 is protected with a 9-fluorenylmethoxycarbonyl (Fmoc) protecting group;
the side chain of the Trp residue at position 1 is protected with a tert-butyloxycarbonyl (Boc) protecting group;
the side chain of the Lys residue at position 4 is protected with a tert-butyloxycarbonyl (Boc), allyloxycarbonyl (Alloc), trityl (Trt), 4-methyltrityl (Mtt), or benzyloxycarbonyl (Cbz) protecting group;
the side chain of the Ser residue at position 12, position 17, or position 22 is protected with a tert-butyl (tBu), O-trityl (OTrt), O-allyl (OAll), or pseudoproline (YPro) protecting group; and
the side chain of the Lys residue at position 23 is protected with a 1-(4,4-dimethyl-2,6-dioxocyclohex-1-ylidene)ethyl (Dde) or 1-(4,4-dimethyl-2,6-dioxocyclohex-1-ylidene)-3-methylbutyl (ivDde) protecting group. In some embodiments, the disclosure provides a protected variant of Peptide E, wherein:
the N-terminus of the Trp residue at position 1 is protected with a 9-fluorenylmethoxycarbonyl (Fmoc) protecting group;
the side chain of the Trp residue at position 1 is protected with a tert-butyloxycarbonyl (Boc) protecting group;
the side chain of the Lys residue at position 4 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), or 4-methyltrityl (Mtt) protecting group;
the side chain of the Ser residue at position 12, position 17, or position 22 is protected with a tert-butyl (tBu), O-trityl (OTrt), O-allyl (OAll), or pseudoproline (Pro) protecting group; and
the side chain of the Lys residue at position 23 is protected with a benzyloxycarbonyl (Cbz) or chlorobenzyloxycarbonyl(2-Cl—Z) protecting group.

In some embodiments, the disclosure provides a protected variant of Peptide E, wherein:
the side chain of the Trp residue at position 1 is protected with a formyl (For) protecting group; and/or
the side chain of the Lys residue at position 4 or position 23 is protected with a benzyloxycarbonyl (Cbz), 2-chlorobenzyloxycarbonyl(2-Cl—Z), or allyloxycarbonyl (Alloc) protecting group; and/or
the side chain of the Ser residue at position 12, position 17, or position 22 is protected with a p-bromobenzyloxycarbonyl (Br—Z), O-benzyl (OBn), or O-allyl (OAll) protecting group.

In some embodiments, the N-terminus of the Trp residue at position 1 is protected with a tert-butyloxycarbonyl (Boc) protecting group.

In some embodiments, the disclosure provides a protected variant of Peptide E, wherein:
the side chain of the Trp residue at position 1 is protected with a formyl (For) protecting group; and/or
the side chain of the Lys residue at position 4 or position 23 is protected with a benzyloxycarbonyl (Cbz) or 2-chlorobenzyloxycarbonyl(2-Cl—Z) protecting group; and/or
the side chain of the Ser residue at position 12, position 17, or position 22 is protected with an O-benzyl (OBn) protecting group.

In some embodiments, the N-terminus of the Trp residue at position 1 is protected with a tert-butyloxycarbonyl (Boc) protecting group.

In some embodiments, the disclosure provides a protected variant of Peptide E, wherein:
the N-terminus of the Trp residue at position 1 is protected with a tert-butyloxycarbonyl (Boc) protecting group;
the side chain of the Trp residue at position 1 is protected with a formyl (For) protecting group;
the side chain of the Lys residue at position 4 is protected with a benzyloxycarbonyl (Cbz) or 2-chlorobenzyloxycarbonyl(2-Cl—Z) protecting group;
the side chain of the Ser residue at position 12, position 17, or position 22 is protected with a p-bromobenzyloxycarbonyl (Br—Z) or O-benzyl (OBn) protecting group; and
the side chain of the Lys residue at position 23 is protected with an allyloxycarbonyl (Alloc) protecting group.

In some embodiments, the disclosure provides a protected variant of Peptide E, wherein:
the N-terminus of the Trp residue at position 1 is protected with a tert-butyloxycarbonyl (Boc) protecting group;
the side chain of the Trp residue at position 1 is protected with a formyl (For) protecting group;
the side chain of the Lys residue at position 4 is protected with a benzyloxycarbonyl (Cbz), 2-chlorobenzyloxycarbonyl(2-Cl—Z), or allyloxycarbonyl (Alloc) protecting group;
the side chain of the Ser residue at position 12, position 17, or position 22 is protected with a p-bromobenzyloxycarbonyl (Br—Z), O-benzyl (OBn), or O-allyl (OAll) protecting group; and
the side chain of the Lys residue at position 23 is protected with a 1-(4,4-dimethyl-2,6-dioxocyclohex-1-ylidene)ethyl (Dde) or 1-(4,4-dimethyl-2,6-dioxocyclohex-1-ylidene)-3-methylbutyl (ivDde) protecting group.

In some embodiments, the disclosure provides a protected variant of Peptide E, wherein:
the N-terminus of the Trp residue at position 1 is protected with a tert-butyloxycarbonyl (Boc) protecting group;
the side chain of the Trp residue at position 1 is protected with a formyl (For) protecting group;
the side chain of the Lys residue at position 4 is protected with an allyloxycarbonyl (Alloc) protecting group;
the side chain of the Ser residue at position 12, position 17, or position 22 is protected with an O-allyl (OAll) protecting group; and
the side chain of the Lys residue at position 23 is protected with a benzyloxycarbonyl (Cbz) or chlorobenzyloxycarbonyl(2-Cl—Z) protecting group.

Certain aspects provide a protected variant of Peptide E, wherein the Trp residue at position 1 is as t-butyloxy carbonyl amide (Boc). Certain aspects describe a protected variant of Peptide E, wherein the Lys residue at position 4 is protected as t-butyloxy carbonyl amide (Boc). Certain aspects describe a protected variant of Peptide E, wherein the Ser residue at position 12 is protected as a t-butyl ether (tBu). Certain aspects provide a protected variant of Peptide E, wherein the Ser residue at position 17 is protected as a t-butyl ether (tBu). Certain aspects provide a protected variant of Peptide E, wherein the Ser residue at position 22 is protected as a t-butyl ether (tBu). Certain aspects provide a protected variant of Peptide E, wherein the Lys residue at position 23 is protected as a N-ε-1-(4,4-dimethyl-2,6-dioxocyclohex-1-ylidene)-3-methylbutyl amine (ivDde).

In some embodiments, the present disclosure provides a protected variant of Peptide E, wherein:
the Trp residue at position 1 is protected with an Fmoc protecting group at the N-terminus; and/or
the Trp residue at position 1 is protected as t-butyloxy carbonyl amide (Boc); and/or
the Lys residue at position 4 is protected as t-butyloxy carbonyl amide (Boc); and/or
the Ser residue at position 12 is protected as a t-butyl ether (tBu); and/or
the Ser residue at position 17 is protected as a t-butyl ether (tBu); and/or
the Ser residue at position 22 is protected as a t-butyl ether (tBu); and/or
the Lys residue at position 23 is protected as a N-ε-1-(4,4-dimethyl-2,6-dioxocyclohex-1-ylidene)-3-methylbutyl amine (ivDde).

In some embodiments, the present disclosure provides a protected variant of Peptide E, wherein each of the Ser residues at positions 12, 17, and 22 is protected as a t-butyl ether (tBu).

In some embodiments, the present disclosure provides a protected variant of Peptide E, wherein:
the Trp residue at position 1 is as t-butyloxy carbonyl amide (Boc);
the Lys residue at position 4 is protected as t-butyloxy carbonyl amide (Boc);
the Ser residue at position 12 is protected as a t-butyl ether (tBu);
the Ser residue at position 17 is protected as a t-butyl ether (tBu);
the Ser residue at position 22 is protected as a t-butyl ether (tBu); and
the Lys residue at position 23 is protected as a N-ε-1-(4,4-dimethyl-2,6-dioxocyclohex-1-ylidene)-3-methylbutyl amine (ivDde).

In some embodiments, the Trp residue at position 1 is protected with an Fmoc protecting group at the N-terminus.

In some embodiments, the protected variant of Peptide E does not have a free C-terminal carboxyl group. In some embodiments, the protected variant of Peptide E has a protected C-terminus. In some embodiments, the C-terminus of the protected variant of Peptide E is amidated.

In some embodiments, the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide E does not have a free C-terminal carboxyl group (SEQ ID NO: 66), or a salt (e.g., an acid-addition salt) thereof.

In some embodiments, the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide E has a protected C-terminus (SEQ ID NO: 159), or a salt (e.g., an acid-addition salt) thereof. In some embodiments, the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 160) or a salt (e.g., an acid-addition salt) thereof.

In some embodiments, the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide E has a protected N-terminus and does not have a free C-terminal carboxyl group (SEQ ID NO: 74). In some embodiments, the protected variant of Peptide E is Fmoc-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide E does not have a free C-terminal carboxyl group (SEQ ID NO: 82).

In some embodiments, the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide E has a protected N-terminus and a protected C-terminus (SEQ ID NO: 162). In some embodiments, the protected variant of Peptide E is Fmoc-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide E has a protected C-terminus (SEQ ID NO: 163).

In some embodiments, the protected variant of Peptide E is Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂, wherein the protected variant of Peptide E has a protected N-terminus (SEQ ID NO: 161). In some embodiments, the protected variant of Peptide E is Fmoc-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 164).

Certain aspects provide a protected variant of Peptide E, having a structure of

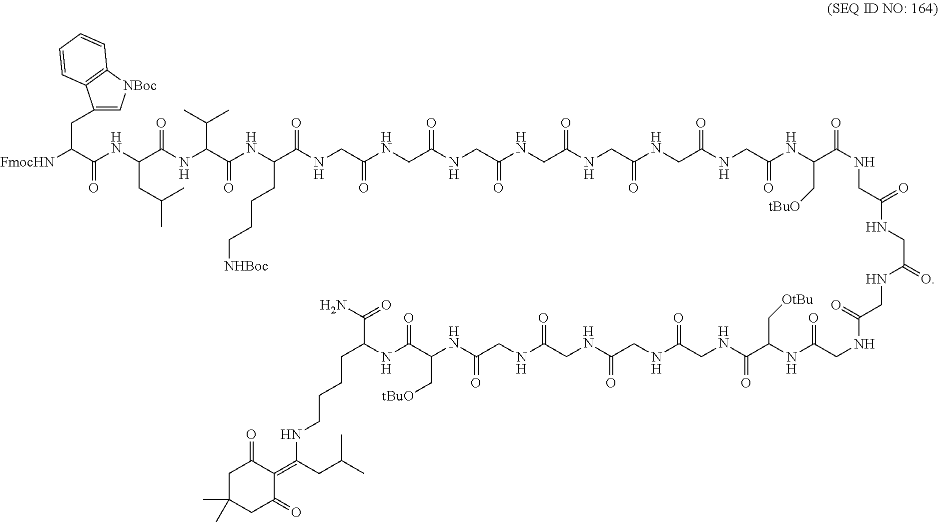

In some embodiments, the protected variant of Peptide E has the structure

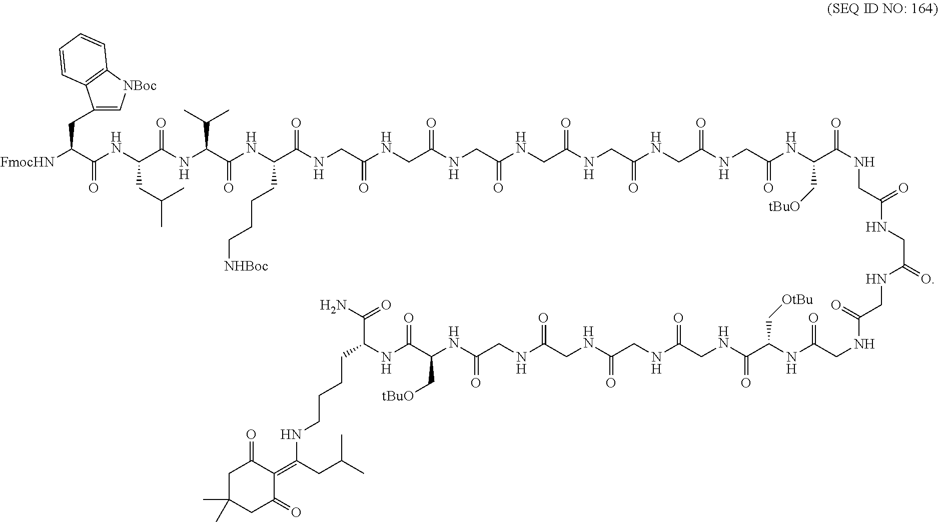

Certain aspects provide a protected variant of Peptide E, having a structure of

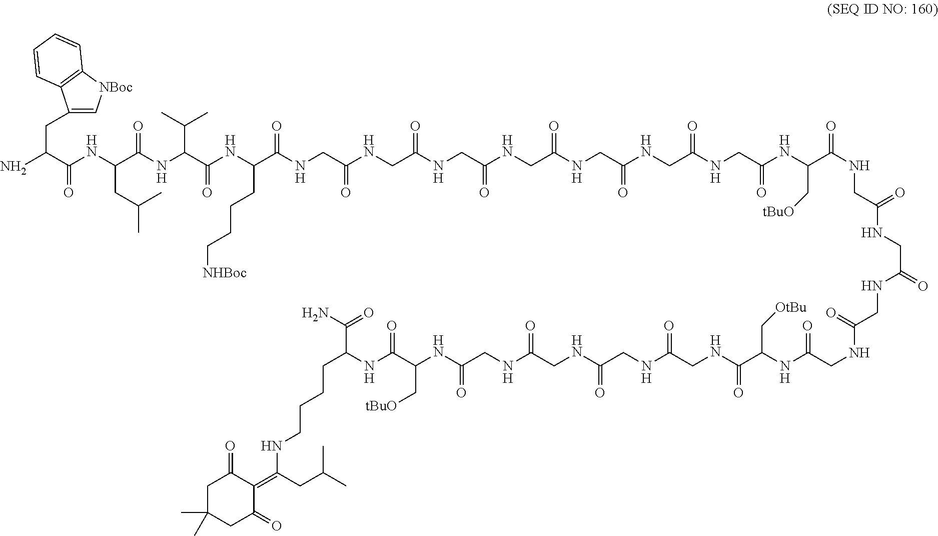 [SMILES: CC(C)CC(NCCCCC(NC(=O)C(COC(C)(C)C)NC(=O)CNC(=O)CNC(=O)CNC(=O)CNC(=O)C(COC(C)(C)C)NC(=O)CNC(=O)CNC(=O)CNC(=O)CNC(=O)C(COC(C)(C)C)NC(=O)CNC(=O)CNC(=O)CNC(=O)CNC(=O)CNC(=O)CNC(=O)CNC(=O)C(CCCCNC(=O)OC(C)(C)C)NC(=O)C(NC(=O)C(CC(C)C)NC(=O)C(N)Cc1cn(C(=O)OC(C)(C)C)c2ccccc12)C(C)C)C(N)=O)=C1C(=O)CC(C)(C)CC1=O]

or a salt (e.g., an acid-addition salt) thereof.

In some embodiments, the protected variant of Peptide E has the structure

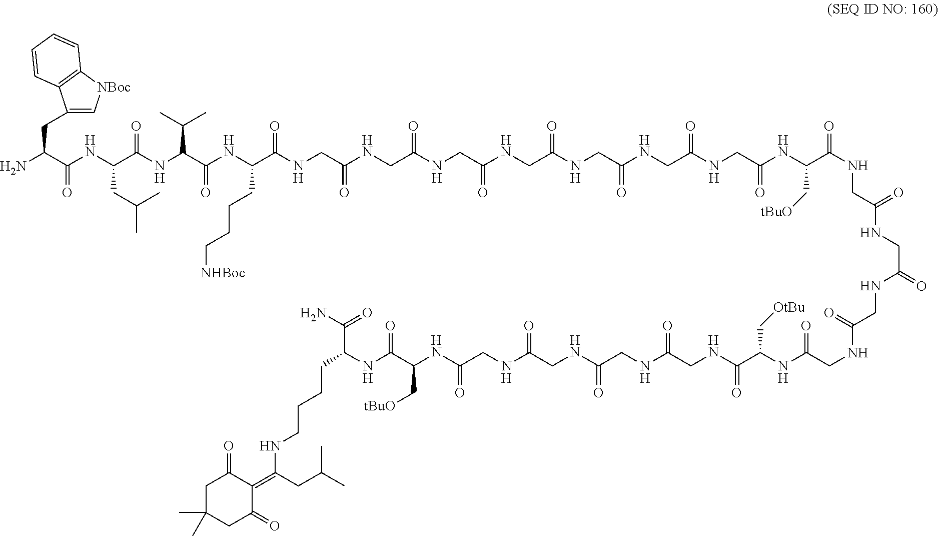

or a salt (e.g., an acid-addition salt) thereof.

Certain aspects provide a conjugate of Peptide E or a protected variant thereof, wherein the Lys residue at position 23 is coupled to a solid support. Solid supports for solid phase synthesis are known in the art. In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing. In some embodiments, the conjugate further comprises a linker through which the peptide is attached to the solid support (e.g., the resin material). Linkers for solid support are known in the art. In some embodiments, the linker is a rink-amide linker or a Sieber-amide linker.

In some embodiments, the solid support is a swellable resin material, and the conjugate further comprises a linker through which the peptide is attached to the swellable resin material.

In some embodiments, the solid support is a swellable resin material, and the conjugate further comprises a linker through which the peptide is attached to the swellable resin material, wherein the linker is a rink-amide linker. In some embodiments, the solid support is a swellable resin material, and the conjugate further comprises a linker through which the peptide is attached to the swellable resin material, wherein the linker is a Sieber-amide linker.

In some embodiments, the solid support is a Rink amide, peptide amide linker (PAL), safety-catch amide linker (SCAL), or Sieber amide resin. In some embodiments, the N-terminus of the conjugate is protected with a 9-fluorenylmethoxycarbonyl (Fmoc) protecting group, and the solid support is a Rink amide, peptide amide linker (PAL), safety-catch amide linker (SCAL), or Sieber amide resin.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin, a 2-chlorodiphenylmethyl resin, a 4-(hydroxymethyl) phenoxyacetic acid (HMPA) resin, a 4-(4-hydroxymethyl-3-methoxyphenoxy) butyric acid (HMPB) resin, or a resin comprising a dialkoxybenzyl-type benzyl alcohol linker on a polystyrene support. In some embodiments, the N-terminus of the conjugate is protected with a 9-fluorenylmethoxycarbonyl (Fmoc) protecting group, and the solid support is a Wang (p-benzyloxybenzyl) resin, a 2-chlorodiphenylmethyl resin, a 4-(hydroxymethyl) phenoxyacetic acid (HMPA) resin, a 4-(4-hydroxymethyl-3-methoxyphenoxy) butyric acid (HMPB) resin, or a resin comprising a dialkoxybenzyl-type benzyl alcohol linker on a polystyrene support.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol-grafted polystyrene. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

In some embodiments, the solid support is a Merrifield (benzyl ester) resin, a hydroxymethyl polystyrene (HMP) resin, a benzhydrylamine (BHA) resin, a p-methylbenzhydrylamine (MBHA) resin, or a phenylacetamidomethyl (PAM) resin. In some embodiments, the N-terminus of the conjugate is protected with a tert-butyloxycarbonyl (Boc) protecting group, and the solid support is a Merrifield (benzyl ester) resin, a hydroxymethyl polystyrene (HMP) resin, a benzhydrylamine (BHA) resin, a p-methylbenzhydrylamine (MBHA) resin, or a phenylacetamidomethyl (PAM) resin.

In some embodiments, the solid support is a Merrifield (benzyl ester) resin. In some embodiments, the solid support is a hydroxymethyl polystyrene (HMP) resin. In some embodiments, the solid support is a benzhydrylamine (BHA) resin. In some embodiments, the solid support is a p-Methylbenzhydrylamine (MBHA) resin. In some embodiments, the solid support is a phenylacetamidomethyl (PAM) resin.

Certain aspects provide a conjugate of a protected variant of Peptide E, having a structure of

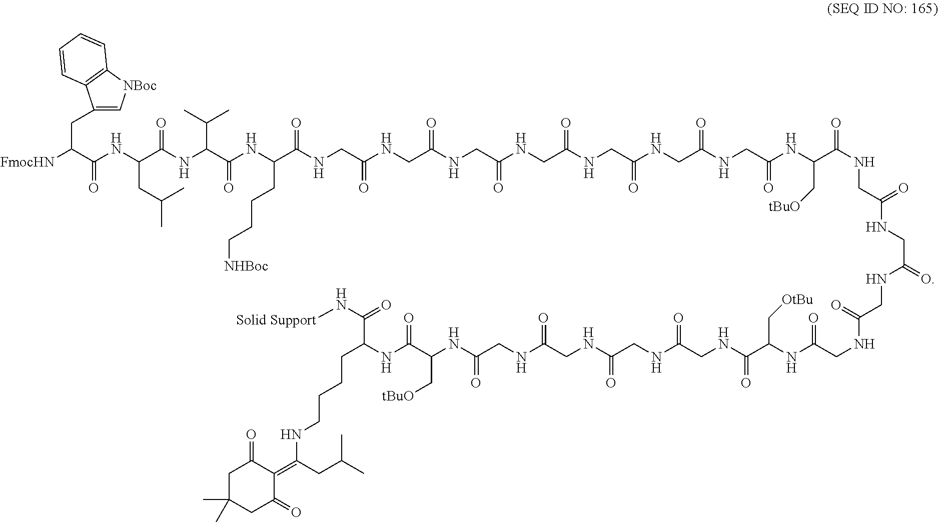

In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

In some embodiments, the conjugate of a protected variant of Peptide E has the structure

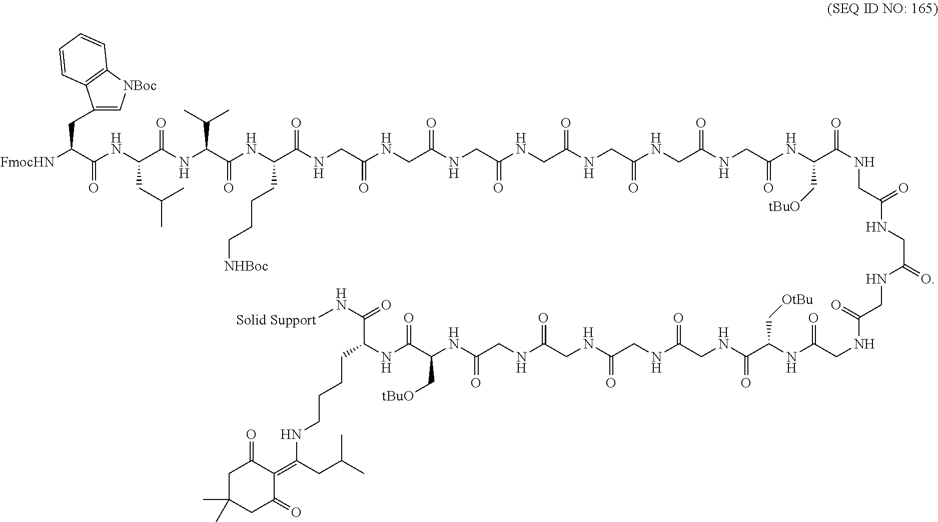

In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol-grafted polystyrene. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

### Peptide F

Also provided herein is a peptide having an amino acid sequence of Tyr-Leu-Glu-Glu-Gln-Ala-Ala-Lys-Glu-Phe-Ile-Ala-Trp-Leu-Val-Lys-Gly-Gly-Gly-Gly-Gly-Gly-Gly-Ser-Gly-Gly-Gly-Gly-Ser-Gly-Gly-Gly-Gly-Ser-Lys (SEQ ID NO: 14) (“Peptide F”). Certain aspects provide a protected variant of Peptide F, wherein at least one of the Tyr, Glu, Gln, Lys, Trp, and Ser residues is protected with a protecting group. In addition, certain aspects provide a protected variant of Peptide F, wherein the Lys residue at position 35 is protected with a side chain protecting group that can be selectively removed (i.e., is orthogonal to any other side chain or termini protecting group(s), such as, e.g., a 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), benzyloxycarbonyl (Cbz), or chlorobenzyloxycarbonyl(2-Cl—Z) protecting group) to enable selective modification of the &-amino group of the C-terminal Lys residue.

In some embodiments, the disclosure provides a protected variant of Peptide F, wherein:
the side chain of the Tyr residue at position 1 is protected with a tert-butyl (tBu), O-allyl (OAll), trityl (Trt), or 2-chlorotrityl (Clt) protecting group; and/or
the side chain of the Glu residue at position 3, position 4, or position 9 is protected with a tert-butyl ester (OtBu), O-2-phenylisopropyl ester (OPp), or allyl ester (OAll) protecting group; and/or
the side chain of the Gln residue at position 5 is protected with a trityl (Trt), benzyloxymethyl (Bom), xanthenyl (Xan), or p-methoxybenzyl (PMB) protecting group; and/or
the side chain of the Lys residue at position 8, position 16, or position 35 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), 4-methyltrityl (Mtt), or benzyloxycarbonyl (Cbz) protecting group; and/or
the side chain of the Trp residue at position 13 is protected with a tert-butyloxycarbonyl (Boc) protecting group; and/or
the side chain of the Ser residue at position 24, position 29, or position 34 is protected with a tert-butyl (tBu), O-trityl (OTrt), O-allyl (OAll), or pseudoproline (YPro) protecting group.

In some embodiments, the N-terminus of the Tyr residue at position 1 is protected with a 9-fluorenylmethoxycarbonyl (Fmoc) protecting group.

In some embodiments, the disclosure provides a protected variant of Peptide F, wherein:
the side chain of the Tyr residue at position 1 is protected with a tert-butyl (tBu) protecting group; and/or
the side chain of the Glu residue at position 3, position 4, or position 9 is protected with a tert-butyl ester (OtBu) protecting group; and/or
the side chain of the Gln residue at position 5 is protected with a trityl (Trt) protecting group; and/or
the side chain of the Lys residue at position 8, position 16, or position 35 is protected with a tert-butyloxycarbonyl (Boc), 1-(4,4-dimethyl-2,6-dioxocyclohex-1-ylidene)ethyl (Dde), or allyloxycarbonyl (Alloc) protecting group; and/or
the side chain of the Trp residue at position 13 is protected with a tert-butyloxycarbonyl (Boc) protecting group; and/or
the side chain of the Ser residue at position 24, position 29, or position 34 is protected with a tert-butyl (tBu) or O-trityl (OTrt) protecting group.

In some embodiments, the N-terminus of the Tyr residue at position 1 is protected with a 9-fluorenylmethoxycarbonyl (Fmoc) protecting group.

In some embodiments, the disclosure provides a protected variant of Peptide F, wherein:
the side chain of the Tyr residue at position 1 is protected with a tert-butyl (tBu), trityl (Trt), or 2-chlorotrityl (Clt) protecting group;
the side chain of the Glu residue at position 3, position 4, or position 9 is protected with a tert-butyl ester (OtBu) or O-2-phenylisopropyl ester (OPp) protecting group;
the side chain of the Gln residue at position 5 is protected with a trityl (Trt), benzyloxymethyl (Bom), xanthenyl (Xan), or p-methoxybenzyl (PMB) protecting group;
the side chain of the Lys residue at position 8 or position 16 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), trityl (Trt), 4-methyltrityl (Mtt), or benzyloxycarbonyl (Cbz) protecting group;
the side chain of the Trp residue at position 13 is protected with a tert-butyloxycarbonyl (Boc) protecting group;
the side chain of the Ser residue at position 24, position 29, or position 34 is protected with a tert-butyl (tBu), O-trityl (OTrt), or pseudoproline (YPro) protecting group; and
the side chain of the Lys residue at position 35 is protected with an allyloxycarbonyl (Alloc) protecting group.

In some embodiments, the disclosure provides a protected variant of Peptide F, wherein:
the side chain of the Tyr residue at position 1 is protected with a tert-butyl (tBu), O-allyl (OAll), trityl (Trt), or 2-chlorotrityl (Clt) protecting group;
the side chain of the Glu residue at position 3, position 4, or position 9 is protected with a tert-butyl ester (OtBu), O-2-phenylisopropyl ester (OPp), or allyl ester (OAll) protecting group;
the side chain of the Gln residue at position 5 is protected with a trityl (Trt), benzyloxymethyl (Bom), xanthenyl (Xan), or p-methoxybenzyl (PMB) protecting group;
the side chain of the Lys residue at position 8 or position 16 is protected with a tert-butyloxycarbonyl (Boc), allyloxycarbonyl (Alloc), trityl (Trt), 4-methyltrityl (Mtt), or benzyloxycarbonyl (Cbz) protecting group;
the side chain of the Trp residue at position 13 is protected with a tert-butyloxycarbonyl (Boc) protecting group;
the side chain of the Ser residue at position 24, position 29, or position 34 is protected with a tert-butyl (tBu), O-trityl (OTrt), O-allyl (OAll), or pseudoproline (YPro) protecting group; and
the side chain of the Lys residue at position 35 is protected with a 1-(4,4-dimethyl-2,6-dioxocyclohex-1-ylidene)ethyl (Dde) or 1-(4,4-dimethyl-2,6-dioxocyclohex-1-ylidene)-3-methylbutyl (ivDde) protecting group. In some embodiments, the disclosure provides a protected variant of Peptide F, wherein:
the side chain of the Tyr residue at position 1 is protected with a tert-butyl (tBu), O-allyl (OAll), trityl (Trt), or 2-chlorotrityl (Clt) protecting group;
the side chain of the Glu residue at position 3, position 4, or position 9 is protected with a tert-butyl ester (OtBu), O-2-phenylisopropyl ester (OPp), or allyl ester (OAll) protecting group;
the side chain of the Gln residue at position 5 is protected with a trityl (Trt), benzyloxymethyl (Bom), xanthenyl (Xan), or p-methoxybenzyl (PMB) protecting group;
the side chain of the Lys residue at position 8 or position 16 is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), or 4-methyltrityl (Mtt) protecting group;
the side chain of the Trp residue at position 13 is protected with a tert-butyloxycarbonyl (Boc) protecting group;
the side chain of the Ser residue at position 24, position 29, or position 34 is protected with a tert-butyl (tBu), O-trityl (OTrt), O-allyl (OAll), or pseudoproline (YPro) protecting group; and
the side chain of the Lys residue at position 35 is protected with a benzyloxycarbonyl (Cbz) or chlorobenzyloxycarbonyl(2-Cl—Z) protecting group.

In some embodiments, the disclosure provides a protected variant of Peptide F, wherein:
the side chain of the Tyr residue at position 1 is protected with an O-benzyl (OBn), O-allyl (OAll), O-(2-bromobenzyl), or 2,6-dichlorobenzyl (Dcb) protecting group; and/or
the side chain of the Glu residue at position 3, position 4, or position 9 is protected with an allyl ester (OAll), benzyl ester (OBn), or cyclohexyl ester (OcHex) protecting group; and/or
the side chain of the Lys residue at position 8, position 16, or position 35 is protected with a benzyloxycarbonyl (Cbz), 2-chlorobenzyloxycarbonyl(2-Cl—Z), or allyloxycarbonyl (Alloc) protecting group; and/or
the side chain of the Trp residue at position 13 is protected with a formyl (For) protecting group; and/or
the side chain of the Ser residue at position 24, position 29, or position 34 is protected with a p-bromobenzyloxycarbonyl (Br—Z), O-benzyl (OBn), or O-allyl (OAll) protecting group.

In some embodiments, the N-terminus of the Tyr residue at position 1 is protected with a tert-butyloxycarbonyl (Boc) protecting group.

In some embodiments, the disclosure provides a protected variant of Peptide F, wherein:
the side chain of the Tyr residue at position 1 is protected with an O-benzyl (OBn) protecting group; and/or the side chain of the Glu residue at position 3, position 4, or position 9 is protected with a benzyl ester (OBn) or allyl ester (OAll) protecting group; and/or
the side chain of the Lys residue at position 8, position 16, or position 35 is protected with a benzyloxycarbonyl (Cbz) or 2-chlorobenzyloxycarbonyl(2-Cl—Z) protecting group; and/or
the side chain of the Trp residue at position 13 is protected with a formyl (For) protecting group; and/or
the side chain of the Ser residue at position 24, position 29, or position 34 is protected with an O-benzyl (OBn) protecting group.

In some embodiments, the N-terminus of the Tyr residue at position 1 is protected with a tert-butyloxycarbonyl (Boc) protecting group.

In some embodiments, the disclosure provides a protected variant of Peptide F, wherein:
the N-terminus of the Tyr residue at position 1 is protected with a tert-butyloxycarbonyl (Boc) protecting group;
the side chain of the Tyr residue at position 1 is protected with an O-benzyl (OBn), O-(2-bromobenzyl), or 2,6-dichlorobenzyl (Dcb) protecting group;
the side chain of the Glu residue at position 3, position 4, or position 9 is protected with a benzyl ester (OBn) or cyclohexyl ester (OcHex) protecting group;
the side chain of the Lys residue at position 8 or position 16 is protected with a benzyloxycarbonyl (Cbz) or 2-chlorobenzyloxycarbonyl(2-Cl—Z) protecting group;
the side chain of the Trp residue at position 13 is protected with a formyl (For) protecting group;
the side chain of the Ser residue at position 24, position 29, or position 34 is protected with a p-bromobenzyloxycarbonyl (Br—Z) or O-benzyl (OBn) protecting group; and
the side chain of the Lys residue at position 35 is protected with an allyloxycarbonyl (Alloc) protecting group.

In some embodiments, the disclosure provides a protected variant of Peptide F, wherein:
the N-terminus of the Tyr residue at position 1 is protected with a tert-butyloxycarbonyl (Boc) protecting group;
the side chain of the Tyr residue at position 1 is protected with an O-benzyl (OBn), O-allyl (OAll), O-(2-bromobenzyl), or 2,6-dichlorobenzyl (Dcb) protecting group;
the side chain of the Glu residue at position 3, position 4, or position 9 is protected with an allyl ester (OAll), benzyl ester (OBn), or cyclohexyl ester (OcHex) protecting group;
the side chain of the Lys residue at position 8 or position 16 is protected with a benzyloxycarbonyl (Cbz), 2-chlorobenzyloxycarbonyl(2-Cl—Z), or allyloxycarbonyl (Alloc) protecting group;
the side chain of the Trp residue at position 13 is protected with a formyl (For) protecting group;
the side chain of the Ser residue at position 24, position 29, or position 34 is protected with a p-bromobenzyloxycarbonyl (Br—Z), O-benzyl (OBn), or O-allyl (OAll) protecting group; and
the side chain of the Lys residue at position 35 is protected with a 1-(4,4-dimethyl-2,6-dioxocyclohex-1-ylidene)ethyl (Dde) or 1-(4,4-dimethyl-2,6-dioxocyclohex-1-ylidene)-3-methylbutyl (ivDde) protecting group.

In some embodiments, the disclosure provides a protected variant of Peptide F, wherein:
the N-terminus of the Tyr residue at position 1 is protected with a tert-butyloxycarbonyl (Boc) protecting group;
the side chain of the Tyr residue at position 1 is protected with an O-allyl (OAll) protecting group;
the side chain of the Glu residue at position 3, position 4, or position 9 is protected with an allyl ester (OAll) or cyclohexyl ester (OcHex) protecting group;
the side chain of the Lys residue at position 8 or position 16 is protected with an allyloxycarbonyl (Alloc) protecting group;
the side chain of the Trp residue at position 13 is protected with a formyl (For) protecting group;
the side chain of the Ser residue at position 24, position 29, or position 34 is protected with an O-allyl (OAll) protecting group; and
the side chain of the Lys residue at position 35 is protected with a benzyloxycarbonyl (Cbz) or chlorobenzyloxycarbonyl(2-Cl—Z) protecting group.

Certain aspects provide a protected variant of Peptide F, wherein the Tyr residue at position 1 is protected with an Fmoc protecting group at the N-terminus. Certain aspects provide a protected variant of Peptide F, wherein the Tyr residue at position 1 is protected as a t-butyl ether (tBu). Certain aspects provide a protected variant of Peptide F, wherein the Glu residue at position 3 is protected as a t-butyl ester (OtBu). Certain aspects provide a protected variant of Peptide F, wherein the Glu residue at position 4 is protected as a t-butyl ester (OtBu). Certain aspects provide a protected variant of Peptide F, wherein the Gln residue at position 5 is protected as a triphenylmethyl amide (Trt). Certain aspects provide a protected variant of Peptide F, wherein the Lys residue at position 8 is protected as a t-butyloxy carbonyl amide (Boc). Certain aspects provide a protected variant of Peptide F, wherein the Glu residue at position 9 is protected as a t-butyl ester (OtBu). Certain aspects provide a protected variant of Peptide F, wherein the Trp residue at position 13 is protected as a t-butyloxy carbonyl amide (Boc). Certain aspects provide a protected variant of Peptide F, wherein the Lys residue at position 16 is protected as t-butyloxy carbonyl amide (Boc). Certain aspects provide a protected variant of Peptide F, wherein the Ser residue at position 24 is protected as a t-butyl ether (tBu). Certain aspects provide a protected variant of Peptide F, wherein the Ser residue at position 29 is protected as a t-butyl ether (tBu). Certain aspects provide a protected variant of Peptide F, wherein the Ser residue at position 34 is protected as a t-butyl ether (tBu). Certain aspects provide a protected variant of Peptide F, wherein the Lys residue at position 35 is protected as a N-ε-1-(4,4-dimethyl-2,6-dioxocyclohex-1-ylidene)-3-methylbutyl amine (ivDde).

In some embodiments, the present disclosure provides a protected variant of Peptide F, wherein:
the Tyr residue at position 1 is protected with an Fmoc protecting group at the N-terminus; and/or
the Tyr residue at position 1 is protected as a t-butyl ether (tBu); and/or
the Glu residue at position 3 is protected as a t-butyl ester (OtBu); and/or
the Glu residue at position 4 is protected as a t-butyl ester (OtBu); and/or
the Gln residue at position 5 is protected as a triphenylmethyl amide (Trt); and/or
the Lys residue at position 8 is protected as a t-butyloxy carbonyl amide (Boc); and/or
the Glu residue at position 9 is protected as a t-butyl ester (OtBu); and/or
the Trp residue at position 13 is protected as a t-butyloxy carbonyl amide (Boc); and/or
the Lys residue at position 16 is protected as t-butyloxy carbonyl amide (Boc); and/or
the Ser residue at position 24 is protected as a t-butyl ether (tBu); and/or
the Ser residue at position 29 is protected as a t-butyl ether (tBu); and/or
the Ser residue at position 34 is protected as a t-butyl ether (tBu); and/or
the Lys residue at position 35 is protected as a N-ε-1-(4,4-dimethyl-2,6-dioxocyclohex-1-ylidene)-3-methylbutyl amine (ivDde).

In some embodiments, the present disclosure provides a protected variant of Peptide F, wherein each of the Ser residues at positions 24, 29, and 34 is protected as a t-butyl ether (tBu).

In some embodiments, the present disclosure provides a protected variant of Peptide F, wherein:
the Tyr residue at position 1 is protected as a t-butyl ether (tBu);
the Glu residue at position 3 is protected as a t-butyl ester (OtBu);
the Glu residue at position 4 is protected as a t-butyl ester (OtBu);
the Gln residue at position 5 is protected as a triphenylmethyl amide (Trt);
the Lys residue at position 8 is protected as a t-butyloxy carbonyl amide (Boc);
the Glu residue at position 9 is protected as a t-butyl ester (OtBu);
the Trp residue at position 13 is protected as a t-butyloxy carbonyl amide (Boc);
the Lys residue at position 16 is protected as t-butyloxy carbonyl amide (Boc);
the Ser residue at position 24 is protected as a t-butyl ether (tBu);
the Ser residue at position 29 is protected as a t-butyl ether (tBu);
the Ser residue at position 34 is protected as a t-butyl ether (tBu); and
the Lys residue at position 35 is protected as a N-ε-1-(4,4-dimethyl-2,6-dioxocyclohex-1-ylidene)-3-methylbutyl amine (ivDde).

In some embodiments, the Tyr residue at position 1 is protected with an Fmoc protecting group at the N-terminus.

In some embodiments, the protected variant of Peptide F does not have a free C-terminal carboxyl group. In some embodiments, the protected variant of Peptide F has a protected C-terminus. In some embodiments, the C-terminus of the protected variant of Peptide F is amidated.

In some embodiments, the protected variant of Peptide F is Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide F does not have a free C-terminal carboxyl group (SEQ ID NO: 67), or a salt (e.g., an acid-addition salt) thereof.

In some embodiments, the protected variant of Peptide F is Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide F has a protected C-terminus (SEQ ID NO: 88), or a salt (e.g., an acid-addition salt) thereof. In some embodiments, the protected variant of Peptide F is Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 85) or a salt (e.g., an acid-addition salt) thereof.

In some embodiments, the protected variant of Peptide F is Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide F has a protected N-terminus and does not have a free C-terminal carboxyl group (SEQ ID NO: 75). In some embodiments, the protected variant of Peptide F is Fmoc-Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide F does not have a free C-terminal carboxyl group (SEQ ID NO: 83).

In some embodiments, the protected variant of Peptide F is Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide F has a protected N-terminus and a protected C-terminus (SEQ ID NO: 89). In some embodiments, the protected variant of Peptide F is Fmoc-Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde), wherein the protected variant of Peptide F has a protected C-terminus (SEQ ID NO: 90).

In some embodiments, the protected variant of Peptide F is Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂, wherein the protected variant of Peptide F has a protected N-terminus (SEQ ID NO: 86). In some embodiments, the protected variant of Peptide F is Fmoc-Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 87).

Certain aspects provide a protected variant of Peptide F, having a structure of

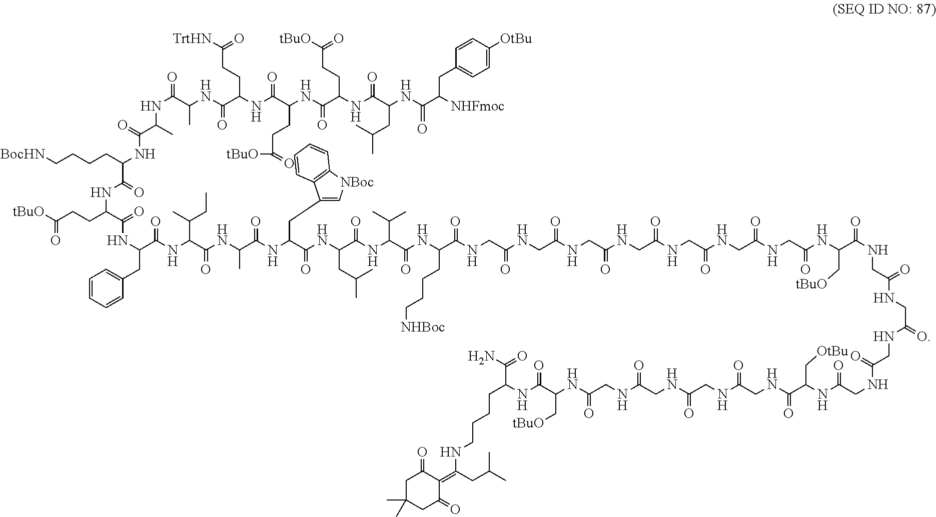

Certain aspects provide a conjugate of Peptide F or a protected variant thereof, wherein the Lys residue at position 35 is coupled to a solid support. Solid supports for solid phase synthesis are known in the art. In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing. In some embodiments, the conjugate further comprises a linker through which the peptide is attached to the solid support (e.g., the resin material). Linkers for solid support are known in the art. In some embodiments, the linker is a rink-amide linker or a Sieber-amide linker.

In some embodiments, the solid support is a swellable resin material, and the conjugate further comprises a linker through which the peptide is attached to the swellable resin material.

In some embodiments, the solid support is a swellable resin material, and the conjugate further comprises a linker through which the peptide is attached to the swellable resin material, wherein the linker is a rink-amide linker. In some embodiments, the solid support is a swellable resin material, and the conjugate further comprises a linker through which the peptide is attached to the swellable resin material, wherein the linker is a Sieber-amide linker.

In some embodiments, the solid support is a Rink amide, peptide amide linker (PAL), safety-catch amide linker (SCAL), or Sieber amide resin. In some embodiments, the N-terminus of the conjugate is protected with a 9-fluorenylmethoxycarbonyl (Fmoc) protecting group, and the solid support is a Rink amide, peptide amide linker (PAL), safety-catch amide linker (SCAL), or Sieber amide resin.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin, a 2-chlorodiphenylmethyl resin, a 4-(hydroxymethyl) phenoxyacetic acid (HMPA) resin, a 4-(4-hydroxymethyl-3-methoxyphenoxy) butyric acid (HMPB) resin, or a resin comprising a dialkoxybenzyl-type benzyl alcohol linker on a polystyrene support. In some embodiments, the N-terminus of the conjugate is protected with a 9-fluorenylmethoxycarbonyl (Fmoc) protecting group, and the solid support is a Wang (p-benzyloxybenzyl) resin, a 2-chlorodiphenylmethyl resin, a 4-(hydroxymethyl) phenoxyacetic acid (HMPA) resin, a 4-(4-hydroxymethyl-3-methoxyphenoxy) butyric acid (HMPB) resin, or a resin comprising a dialkoxybenzyl-type benzyl alcohol linker on a polystyrene support.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol-grafted polystyrene. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

In some embodiments, the solid support is a Merrifield (benzyl ester) resin, a hydroxymethyl polystyrene (HMP) resin, a benzhydrylamine (BHA) resin, a p-methylbenzhydrylamine (MBHA) resin, or a phenylacetamidomethyl (PAM) resin. In some embodiments, the N-terminus of the conjugate is protected with a tert-butyloxycarbonyl (Boc) protecting group, and the solid support is a Merrifield (benzyl ester) resin, a hydroxymethyl polystyrene (HMP) resin, a benzhydrylamine (BHA) resin, a p-methylbenzhydrylamine (MBHA) resin, or a phenylacetamidomethyl (PAM) resin.

In some embodiments, the solid support is a Merrifield (benzyl ester) resin. In some embodiments, the solid support is a hydroxymethyl polystyrene (HMP) resin. In some embodiments, the solid support is a benzhydrylamine (BHA) resin. In some embodiments, the solid support is a p-Methylbenzhydrylamine (MBHA) resin. In some embodiments, the solid support is a phenylacetamidomethyl (PAM) resin.

Certain aspects provide a conjugate of a protected variant of Peptide F, having a structure of

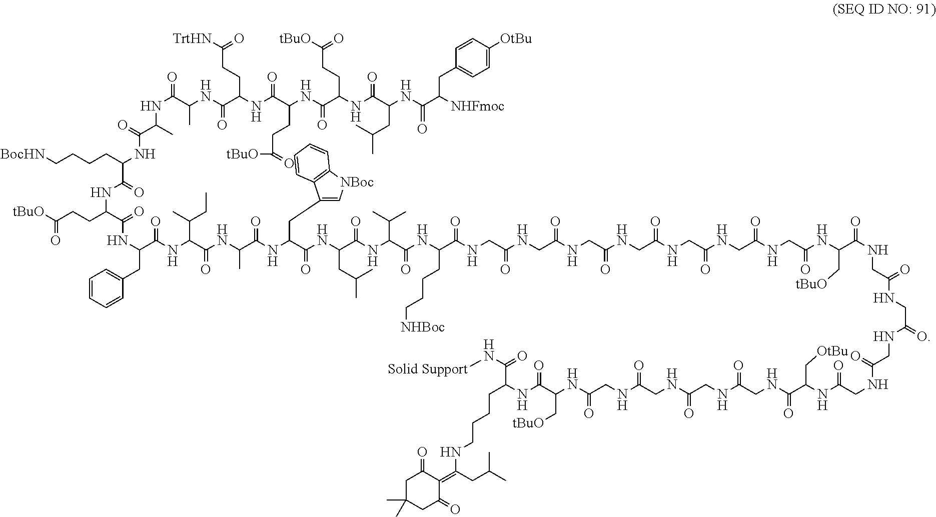

In some embodiments, the solid support is a resin material. In some embodiments, the resin material is a swellable resin material. In some embodiments, the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing.

In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support.

In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support.

In some embodiments, the solid support is a safety-catch amide linker (SCAL) resin.

In some embodiments, the solid support is a Sieber amide resin.

In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol support.

In some embodiments, the solid support is a 2-chlorodiphenylmethyl(2-CTC) resin. In some embodiments, the 2-CTC resin comprises a 2-CTC linker on a cross-linked polystyrene support.

In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support.

In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support.

In some embodiments, the solid support is a resin comprising a dialkoxybenzyl-type benzyl alcohol linker on a polystyrene support.

### Protecting Groups of Side Chain Residues and Termini of Peptides and Fragments Disclosed Herein

As used herein, the term “protecting group” refers to a group used to temporarily mask the characteristic chemistry of a functional group because it may interfere with another reaction. Suitable protecting groups should be easy to put on, easy to remove, and, in high yielding reactions, inert to the required reaction conditions.

The side chains of natural and unnatural amino acids include a variety of functional groups that can result in side-chain reactivity during peptide synthesis. To minimize side chain reactivity, side chain protecting groups can be used. Side chain protecting groups are well-known in the art and designed to withstand multiple peptide elongation cycles. Specifically, side chain protecting groups used in certain methods described herein are stable under 9-fluorenylmethyloxycarbonyl (Fmoc) deprotection conditions, such as exposure to a mild base. Additionally, side chain protecting groups used in some methods described herein are stable under Boc deprotection conditions, such as exposure to TFA (e.g., benzyl-type side chain protecting groups (e.g., benzyl esters/ethers and benzyloxycarbonyl derivatives) that are stable to TFA exposure but can be removed by HF or TFMSA exposure).

In some embodiments, protecting groups (e.g., side chain protecting groups) are selected from moieties that are stable to both Fmoc deprotection conditions (e.g., exposure to piperidine/DMF) and common peptide coupling reagents (e.g., DIC/Oxyma, HATU, TBTU). In certain embodiments, protecting groups (e.g., side chain protecting groups) are selected from moieties that are stable to both Fmoc deprotection conditions (e.g., exposure to piperidine/DMF) and common peptide coupling reagents (e.g., DIC/Oxyma, HATU, TBTU) but can be removed selectively under non-TFA conditions (e.g., Pd(0)-mediated deallylation, hydrazinolysis exposure, hydroxylamine or hydrazine exposure). Such orthogonal protecting groups may be useful in peptide synthesis methods that use an Fmoc protecting group to protect the N-terminus during peptide coupling. For example, in some embodiments, orthogonal protecting groups such as ivDde and Dde can be used to protect the ¿-amino group of the C-terminal lysine residue of Peptide A to allow for site selective functionalization (e.g., bromoacetylation of the C-terminal lysine residue after selective removal of an ivDde or Dde protecting group without removing other side chain protecting groups (e.g., tBu or OtBu groups).

In other embodiments, protecting groups (e.g., side chain protecting groups, C-terminal protecting groups) are selected from moieties that are stable to both Boc deprotection conditions (e.g., exposure to TFA) and common peptide coupling reagents (e.g., DIC/Oxyma, HATU, TBTU). In certain embodiments, protecting groups (e.g., side chain protecting groups, C-terminal protecting groups) are selected from moieties that are stable to both Boc deprotection conditions (e.g., exposure to TFA) and common peptide coupling reagents (e.g., DIC/Oxyma, HATU, TBTU) but can be removed selectively under non-TFA conditions (e.g., Pd(0)-mediated deallylation, hydrazinolysis exposure, hydroxylamine or hydrazine exposure). Such orthogonal protecting groups may be useful in peptide synthesis methods that use a Boc protecting group to protect the N-terminus during peptide coupling.

For example, in some embodiments, side chain protecting groups that can be used in methods of the present disclosure include trityl (Trt), tert-butoxycarbonyl (Boc), tert-butyl (tBu), and benzothiophenesulfone-2-methyloxycarbonyl (Bsmoc).

Non-limiting examples of amino protecting groups include carbamates, amides, N-alkyls, amino acetals, N-benzyls, imines, enamines, N-phosphinyl, N-phosphoryl, N-silyl, N-sulfenyl, and N-sulfonyl. Additional examples of protecting groups include, but are not limited to, monomethoxytrityl (MMT), carbobenzyloxy (Cbz), p-methoxybenzyloxycarbonyl (Moz or MeOZ), t-butyloxycarbonyl (Boc), 9-fluorenylmethyloxycarbonyl (Fmoc), and allyloxycarbamate, acetyl (Ac), benzoyl (Bz), trichloroethyl chloroformate (Troc), p-toluenesulfonyl (Tosyl; Ts), nitrobenzenesulfonyl (Nosyl), 2-nitrophenylsulfenyl (Nps), benzyl (Bn), p-methoxybenzyl (PMB), 3,4-dimethoxybenzyl (DMPM), and p-methoxyphenyl (PMP). A specific N-alkyl group is monomethoxytrityl, which can be cleaved under neutral conditions.

Illustratively, amino protecting groups that can be used in methods of the present disclosure include, but are not limited to, urethane-type protecting groups (e.g., methoxycarbonyl, ethoxycarbonyl, tert-butoxycarbonyl (Boc), benzyloxycarbonyl (Cbz)), acyl-type protecting groups (e.g., formyl, acetyl, trifluoroacetyl), and sulfonyl-type protecting groups (e.g., p-toluenesulfonyl (Ts), p-tolylmethanesulfonyl, 4-methoxy-2,3,6-trimethylbenzenesulfonyl). Other amino protecting groups include 2-acetyl-5,5-dimethyl-cyclohexan-1,3-dione (Dde) and N-ε-1-(4,4-dimethyl-2,6-dioxocyclohex-1-ylidene)-3-methylbutyl amine (ivDde). In some embodiments, amino protecting groups that can be used in methods of the present disclosure include, but are not limited to, N-allyloxycarbonyl (Alloc), 2-(trimethylsilyl) ethoxycarbonyl (Teoc), Cbz, Boc, Fmoc, and Trt.

Hydroxy protecting groups that can be used in the disclosed methods include, but are not limited to, alkyl-type protecting groups (e.g., ethyl, ethyl, tert-butyl (tBu)), alkoxylalkyl-type protecting groups (e.g., methoxymethyl (MOM), 2-tetrahydropyranyl (THP), ethoxyethyl (EE)), acyl-type protecting groups (e.g., acetyl, pivaloyl, benzoyl), alkylsilyl-type protecting groups (e.g., trimethylsilyl (TMS), triethylsilyl (TES), tert-butyldimethylsilyl (TBS, TBDMS), triisopropyl silyl (TIPS), tert-butyldiphenylsilyl (TBDPS)), allyl protecting groups, and o-nitrobenzyl.

Arginine protecting groups that can be used in the disclosed methods include, but are not limited to, p-toluenesulfonyl and 2,2,4,6,7-pentamethyldihydrobenzofuran-5-sulfonyl (Pbf).

Protecting groups for asparagine, glutamine, and histidine that can be used in the disclosed methods include, but are not limited to, trityl and benzyloxymethyl.

Cysteine protecting groups that can be used in the disclosed methods include, but are not limited to, trityl, p-methylbenzyl, and acetamidomethyl.

Tryptophan protecting groups that can be used in the disclosed methods include, but are not limited to, Boc and formyl.

C-terminal protecting groups can also be used, for example, in liquid phase peptide synthesis methods described herein. In some embodiments, a C-terminus of a protected peptide used in an LPPS method described herein is protected as an ester selected from acid-labile, base- or hydrolysis-labile, hydrogenolysis-labile benzyl-type, palladium(0)-cleavable TT-allyl, fluoride-labile silyl-ethyl, photolabile arylacyl/nitrobenzyl, and reductively cleavable haloalkyl esters. In some embodiments, the C-terminus of the protected peptide is protected as a tert-butyl, 2-chlorotrityl, 2,4-dimethoxybenzyl, 2-phenylisopropyl, 9-fluorenylmethyl, 4-(N-[1-(4,4-dimethyl-2,6-dioxocyclohexylidene)-3-methylbutyl]amino)benzyl, methyl, ethyl, carbamoylmethyl, allyl, benzyl, phenacyl, p-nitrobenzyl, 2-trimethylsilylethyl, (2-phenyl-2-trimethylsilyl)ethyl, 2-(trimethylsilyl) isopropyl, 2,2,2-trichloroethyl, p-hydroxyphenacyl, 4,5-dimethoxy-2-nitrobenzyl, or 1,1-dimethylallyl ester.

In some embodiments of the present disclosure, the Lys at the C-terminus of Peptide A may be optionally protected/functionalized as a bromoacetyl moiety.

In some embodiments, an N-terminus of a protected variant of a peptide described herein is protected with a tert-butyloxycarbonyl (Boc), 9-fluorenylmethoxycarbonyl (Fmoc), trityl (Trt), N-allyloxycarbonyl (Alloc), or benzyloxycarbonyl (Cbz) protecting group. In some embodiments, an N-terminus of a protected variant of a peptide described herein is protected with a tert-butyloxycarbonyl (Boc) protecting group. In some embodiments, an N-terminus of a protected variant of a peptide described herein is protected with a 9-fluorenylmethoxycarbonyl (Fmoc) protecting group. In some embodiments, an N-terminus of a protected variant of a peptide described herein is protected with a trityl (Trt) protecting group. In some embodiments, an N-terminus of a protected variant of a peptide described herein is protected with a benzyloxycarbonyl (Cbz) protecting group.

In some embodiments, a side chain of a lysine residue of a protected variant of a peptide described herein is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), 4-methyltrityl (Mtt), or benzyloxycarbonyl (Cbz) protecting group. In some embodiments, an N-terminus of a protected variant of a peptide described herein is protected with a 9-fluorenylmethoxycarbonyl (Fmoc) protecting group, and a side chain of a lysine residue of the protected variant is protected with a tert-butyloxycarbonyl (Boc), 1-(4,4-dimethyl-2,6-dioxocyclohex-1-ylidene)ethyl (Dde), 1-(4,4-dimethyl-2,6-dioxocyclohex-1-ylidene)-3-methylbutyl (ivDde), allyloxycarbonyl (Alloc), trityl (Trt), 4-methyltrityl (Mtt), or benzyloxycarbonyl (Cbz) protecting group.

In some embodiments, a side chain of a lysine residue of a protected variant of a peptide described herein is protected with a benzyloxycarbonyl (Cbz), 2-chlorobenzyloxycarbonyl(2-Cl—Z), or allyloxycarbonyl (Alloc) protecting group. In some embodiments, an N-terminus of a protected variant of a peptide described herein is protected with a tert-butyloxycarbonyl (Boc) protecting group, and a side chain of a lysine residue of the protected variant is protected with a benzyloxycarbonyl (Cbz), 2-chlorobenzyloxycarbonyl(2-Cl—Z), or allyloxycarbonyl (Alloc) protecting group.

In some embodiments, a side chain of a glutamic acid or aspartic acid residue of a protected variant of a peptide described herein is protected with a tert-butyl ester (OtBu), O-2-phenylisopropyl ester (OPp), or allyl ester (OAll) protecting group. In some embodiments, an N-terminus of a protected variant of a peptide described herein is protected with a 9-fluorenylmethoxycarbonyl (Fmoc) protecting group, and a side chain of a glutamic acid or aspartic acid residue of the protected variant is protected with a tert-butyl ester (OtBu), O-2-phenylisopropyl ester (OPp), or allyl ester (OAll) protecting group.

In some embodiments, a side chain of a glutamic acid or aspartic acid residue of a protected variant of a peptide described herein is protected with an allyl ester (OAll), benzyl ester (OBn), or cyclohexyl ester (OcHex) protecting group. In some embodiments, an N-terminus of a protected variant of a peptide described herein is protected with a tert-butyloxycarbonyl (Boc) protecting group, and a side chain of a glutamic acid or aspartic acid residue of the protected variant is protected with an allyl ester (OAll), benzyl ester (OBn), or cyclohexyl ester (OcHex) protecting group.

In some embodiments, a side chain of a serine or threonine residue of a protected variant of a peptide described herein is protected with a tert-butyl (tBu), O-trityl (OTrt), O-allyl (OAll), or pseudoproline (YPro) protecting group. In some embodiments, an N-terminus of a protected variant of a peptide described herein is protected with a 9-fluorenylmethoxycarbonyl (Fmoc) protecting group, and a side chain of a serine or threonine residue of the protected variant is protected with a tert-butyl (tBu), O-trityl (OTrt), O-allyl (OAll), or pseudoproline (YPro) protecting group.

For example, a side chain of a serine or threonine residue is protected with a pseudoproline (YPro) protecting group when a pseudoproline derivative that masks the Ser or Thr side chain as a proline-like five-membered oxazolidine (Ser(Psi(Me,Me)Pro) or Thr (Psi (Me,Me)Pro) herein) is introduced in a peptide sequence. Illustratively, H-Ser(Psi(Me,Me)Pro)-OH (R═H) and H-Thr (Psi (Me,Me)Pro)-OH (R═CH₃) have the following structure:

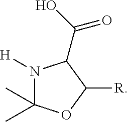

As an additional illustration, Fmoc-Gly-Ser(Psi(Me, Me)Pro)-OH, a dipeptide in which the N-terminus is protected with an Fmoc protecting group and the side chain of the Ser residue is protected with a pseudoproline (YPro) protecting group, has the following structure:

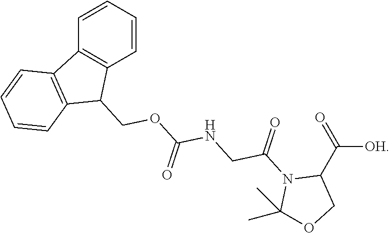 [SMILES: CC1(C)OCC(C(=O)O)N1C(=O)CNC(=O)OCC1c2ccccc2-c2ccccc21]

In some embodiments, a side chain of a serine or threonine residue of a protected variant of a peptide described herein is protected with a p-bromobenzyloxycarbonyl (Br—Z), O-benzyl (OBn), or O-allyl (OAll) protecting group. In some embodiments, an N-terminus of a protected variant of a peptide described herein is protected with a tert-butyloxycarbonyl (Boc) protecting group, and a side chain of a serine or threonine residue of the protected variant is protected with a p-bromobenzyloxycarbonyl (Br—Z), O-benzyl (OBn), or O-allyl (OAll) protecting group.

In some embodiments, a side chain of a tyrosine residue of a protected variant of a peptide described herein is protected with a tert-butyl (tBu), O-allyl (OAll), trityl (Trt), or 2-chlorotrityl (Clt) protecting group. In some embodiments, an N-terminus of a protected variant of a peptide described herein is protected with a 9-fluorenylmethoxycarbonyl (Fmoc) protecting group, and a side chain of a tyrosine residue of the protected variant is protected with a tert-butyl (tBu), O-allyl (OAll), trityl (Trt), or 2-chlorotrityl (Clt) protecting group.

In some embodiments, a side chain of a tyrosine residue of a protected variant of a peptide described herein is protected with an O-benzyl (OBn), O-allyl (OAll), O-(2-bromobenzyl), or 2,6-dichlorobenzyl (Dcb) protecting group. In some embodiments, an N-terminus of a protected variant of a peptide described herein is protected with a tert-butyloxycarbonyl (Boc) protecting group, and a side chain of a tyrosine residue of the protected variant is protected with an O-benzyl (OBn), O-allyl (OAll), O-(2-bromobenzyl), or 2,6-dichlorobenzyl (Dcb) protecting group.

In some embodiments, a side chain of a glutamine residue of a protected variant of a peptide described herein is protected with a trityl (Trt), benzyloxymethyl (Bom), xanthenyl (Xan), or p-methoxybenzyl (PMB) protecting group. In some embodiments, an N-terminus of a protected variant of a peptide described herein is protected with a 9-fluorenylmethoxycarbonyl (Fmoc) protecting group, and a side chain of a glutamine residue of the protected variant is protected with a trityl (Trt), benzyloxymethyl (Bom), xanthenyl (Xan), or p-methoxybenzyl (PMB) protecting group.

In some embodiments, a side chain of a histidine residue of a protected variant of a peptide described herein is protected with a trityl (Trt), tert-butyloxycarbonyl (Boc), mesitylene-2-sulfonyl (Mts), monomethoxytrityl (Mmt), or 4-methyltrityl (Mtt) protecting group. In some embodiments, an N-terminus of a protected variant of a peptide described herein is protected with a 9-fluorenylmethoxycarbonyl (Fmoc) protecting group, and a side chain of a histidine residue of the protected variant is protected with a trityl (Trt), tert-butyloxycarbonyl (Boc), mesitylene-2-sulfonyl (Mts), monomethoxytrityl (Mmt), or 4-methyltrityl (Mtt) protecting group.

In some embodiments, a side chain of a histidine residue of a protected variant of a peptide described herein is protected with a 2,4-dinitrophenyl (Dnp), p-toluenesulfonyl (Tos), o-nitrobenzyl (ONb), or benzyloxymethyl (Bom) protecting group. In some embodiments, an N-terminus of a protected variant of a peptide described herein is protected with a tert-butyloxycarbonyl (Boc) protecting group, and a side chain of a histidine residue of the protected variant is protected with a 2,4-dinitrophenyl (Dnp), p-toluenesulfonyl (Tos), o-nitrobenzyl (ONb), or benzyloxymethyl (Bom) protecting group.

In some embodiments, a side chain of a tryptophan residue of a protected variant of a peptide described herein is protected with a tert-butyloxycarbonyl (Boc) protecting group. In some embodiments, an N-terminus of a protected variant of a peptide described herein is protected with a 9-fluorenylmethoxycarbonyl (Fmoc) protecting group, and a side chain of a tryptophan residue of the protected variant is protected with a tert-butyloxycarbonyl (Boc) protecting group.

In some embodiments, a side chain of a tryptophan residue of a protected variant of a peptide described herein is protected with a formyl (For) protecting group. In some embodiments, an N-terminus of a protected variant of a peptide described herein is protected with a tert-butyloxycarbonyl (Boc) protecting group, and a side chain of a tryptophan residue of the protected variant is protected with a formyl (For) protecting group.

### Solid Phase Peptide Synthesis Solid Supports

The peptides disclosed herein may be immobilized on a solid support. In general, any solid support may be used. Non-limiting examples of solid support materials include swellable resin (e.g., polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing), polystyrene (e.g., in resin form such as microporous polystyrene resin, mesoporous polystyrene resin, macroporous polystyrene resin), glass, polysaccharides (e.g., cellulose, agarose), polyacrylamide resins, polyethylene glycol, or copolymer resins (e.g., comprising polyethylene glycol, polystyrene, etc.). Amide-based resins are particularly useful for the methods of the present disclosure. In some embodiments, the amide-based resins are selected from Rink amide resins, Sieber resins, and xanthenyl resins.

In some embodiments, a peptide disclosed herein with an Fmoc-protected N-terminus may be immobilized on a solid support, wherein the solid support is a Rink amide, peptide amide linker (PAL), safety-catch amide linker (SCAL), or Sieber amide resin. In some embodiments, the solid support is a Rink amide resin. In some embodiments, the Rink amide resin comprises a Rink amide linker on an aminomethyl polystyrene support. In some embodiments, the Rink amide resin comprises a Rink amide linker on a 4-methylbenzhydrylamine polystyrene support. In some embodiments, the Rink amide resin comprises a Rink amide linker on a polyethylene glycol polystyrene support. In some embodiments, the Rink amide resin comprises a Rink amide linker on a polyethylene glycol support. In some embodiments, the Rink amide resin comprises a Rink amide linker on a polyethylene glycol acrylate support. In some embodiments, the solid support is a peptide amide linker (PAL) resin. In some embodiments, the PAL resin comprises a peptide amide linker on a polystyrene support. In some embodiments, the PAL resin comprises a peptide amide linker on a polyethylene glycol polystyrene support. In some embodiments, the solid support is a safety-catch amide linker (SCAL) resin. In some embodiments, the solid support is a Sieber amide resin.

In some embodiments, a peptide disclosed herein with an Fmoc-protected N-terminus may be immobilized on a solid support, wherein the solid support is a Wang (p-benzyloxybenzyl) resin, a 2-chlorodiphenylmethyl resin, a 4-(hydroxymethyl) phenoxyacetic acid (HMPA) resin, a 4-(4-hydroxymethyl-3-methoxyphenoxy) butyric acid (HMPB) resin, or a resin comprising a dialkoxybenzyl-type benzyl alcohol linker on a polystyrene support. In some embodiments, the solid support is a Wang (p-benzyloxybenzyl) resin. In some embodiments, the Wang resin comprises a Wang linker on a cross-linked polystyrene support. In some embodiments, the Wang resin comprises a Wang linker on a polyethylene glycol-grafted polystyrene. In some embodiments, the Wang resin comprises a Wang linker on a polyethylene glycol support. In some embodiments, the solid support is a 2-chlorodiphenylmethyl(2-CTC) resin. In some embodiments, the 2-CTC resin comprises a 2-CTC linker on a cross-linked polystyrene support. In some embodiments, the solid support is a 4-(hydroxymethyl) phenoxyacetic acid (HMPA) resin. In some embodiments, the HMPA resin comprises a HMPA linker on an aminomethyl polystyrene support. In some embodiments, the HMPA resin comprises a HMPA linker on a polyethylene glycol-grafted polystyrene support. In some embodiments, the solid support is a 4-(4-hydroxymethyl-3-methoxyphenoxy) butyric acid (HMPB) resin. In some embodiments, the HMPB resin comprises a HMPB linker on a polystyrene support. In some embodiments, the HMPB resin comprises a HMPB linker on a polyethylene glycol support. In some embodiments, the solid support is a resin comprising a dialkoxybenzyl-type benzyl alcohol linker on a polystyrene support.

In some embodiments, a peptide disclosed herein with a Boc-protected N-terminus may be immobilized on a solid support, wherein the solid support is a Merrifield (benzyl ester) resin, a hydroxymethyl polystyrene (HMP) resin, a benzhydrylamine (BHA) resin, a p-methylbenzhydrylamine (MBHA) resin, or a phenylacetamidomethyl (PAM) resin. In some embodiments, the solid support is a Merrifield (benzyl ester) resin. In some embodiments, the solid support is a hydroxymethyl polystyrene (HMP) resin. In some embodiments, the solid support is a benzhydrylamine (BHA) resin. In some embodiments, the solid support is a p-Methylbenzhydrylamine (MBHA) resin. In some embodiments, the solid support is a phenylacetamidomethyl (PAM) resin.

The solid support may have any suitable form factor. For example, the solid support can be in the form of beads, particles, fibers, or in any other suitable form factor. In some embodiments, the solid support is in the form of beads. In some embodiments, the solid support is in the form of particles. In some embodiments, the solid support is in the form of fibers.

In some embodiments, the solid support may be porous. For example, in some embodiments, macroporous materials (e.g., macroporous polystyrene resins), mesoporous materials, and/or microporous materials (e.g., microporous polystyrene resin) may be employed as a solid support. The terms “macroporous,” “mesoporous,” and “microporous,” when used in relation to solid supports for peptide synthesis, are known to those of ordinary skill in the art and are used herein in consistent fashion with their description in the International Union of Pure and Applied Chemistry (IUPAC) Compendium of Chemical Terminology, Version 2.3.2, Aug. 19, 2012 (informally known as the “Gold Book”). Generally, microporous materials include those having pores with cross-sectional diameters of less than about 2 nanometers. Mesoporous materials include those having pores with cross-sectional diameters of from about 2 nanometers to about 50 nanometers. Macroporous materials include those having pores with cross-sectional diameters of greater than about 50 nanometers and as large as 1 micrometer.

### Solid Phase Peptide Synthesis Coupling Conditions

During solid phase peptide synthesis (SPPS), a peptide can be assembled from the C- to N-terminus on a solid support bearing a cleavable linker through iterative deprotection-coupling-wash cycles. Illustratively, in each cycle, an N-terminal protecting group on an amino acid or peptide immobilized on a solid support may be selectively removed (i.e., removed without deprotecting amino acid side chains with orthogonal side chain protecting groups), followed by coupling with an incoming amino acid or peptide. The C-terminal carboxyl group of the incoming amino acid or peptide can be pre-activated or activated in situ; the activated amino acid or peptide then reacts with the free N-terminal amino group of the immobilized amino acid or peptide to form an amide bond. The N-terminus of the protected amino acid or peptide on solid support can initially be present as either a free α-amino group, or the protected amino acid or peptide can be in the form of an acid-addition salt (such as, e.g., an HCl or TFA salt following acidolytic deprotection). When the amine coupling partner is initially in the form of an acid-addition salt, the protected amino acid or peptide on solid support can be contacted with a non-nucleophilic tertiary base (such as, e.g., DIPEA, NMM, or collidine) to convert the acid-addition salt to a free base, liberating an α-amino group for reaction with a coupling partner with an activated C-terminal carboxyl group. Filtration and wash steps can then be used to remove excess reagents, and the cycle may be repeated until the full peptide chain is assembled on solid support. After assembly, the peptide can globally deprotected and cleaved from the solid support.

In preferred embodiments of solid phase peptide synthesis methods described herein, solid phase peptide coupling conditions (e.g., identity of solid support, identity and amount(s) of coupling reagent, base (if any), and coupling additive (if any); solvent(s); temperature(s); and order of addition, including choice of preactivation vs. in situ activation of a C-terminal carboxyl group) can be selected to limit the occurrence of issues that may arise during peptide coupling (e.g., racemization at the α-stereocenter, guanidination/alkylation of amines, N-acylurea formation, and self-association/aggregation-driven side reaction). Additionally, in preferred embodiments, a peptide is assembled using side chain protecting groups that are compatible with the selected coupling and selective N-terminal protecting group deprotection conditions.

### Solvents

The solid phase peptide coupling reactions of the present disclosure can be carried out in many organic solvents. Preferred solvents are polar, aprotic solvents. Non-limiting example solvents include methylene chloride (DCM), N-methylpyrrolidone (NMP), N,N-dimethylformamide (DMF), N,N-dimethylacetamide (DMA), dimethylsulfoxide (DMSO), acetonitrile, and tetrahydrofuran. Particularly useful solvents in the present disclosure are DMF and DMSO.

Certain aspects of the disclosure provide methods of preparing Peptide A, wherein the solid phase peptide coupling conditions of any one of steps (A), (B), and (C) in multi-step SPPS methods described herein (e.g., Embodiments A.90-A. 103) comprise a coupling reagent in DMF or DMSO. In some embodiments, the solid phase peptide coupling conditions further comprise a base. In some embodiments, the base comprises collidine, triethylamine, diisopropylethylamine, or a combination thereof. In some embodiments, the solid support is an amide-based resin.

### Coupling Reagents, Bases, and Additives

During solid phase peptide synthesis, a coupling reagent can be used to activate the C-terminal carboxyl group of an incoming amino acid or peptide to form an activated/reactive intermediate that facilitates amide bond formation. In some embodiments of methods described herein, activation of the C-terminal carboxyl group can be accomplished through preactivation, wherein the carboxyl group is contacted with a coupling reagent to form an activated intermediate before being in the presence of the coupling partner, or through activation in situ, wherein the activated intermediate is generated directly in the presence of the coupling partner. A base, such as a non-nucleophilic tertiary base (e.g., DIPEA, NMM, or a non-nucleophilic pyridine (e.g., 2,6-lutidine, 2,4,6-trimethylpyridine (“collidine”))), may be used during a coupling reaction to deprotonate the N-terminal amino group (or liberate it when the amine coupling partner is initially present in the form of an acid-addition salt, e.g., following acidolytic deprotection) of the resin-bound amine to increase nucleophilicity and neutralize acids generated during activation of the incoming protected amino acid or peptide, driving the coupling reaction toward completion. In addition, a coupling additive (e.g., HOBt, HOAt) can be used as an acyl-transfer catalyst. As used herein, a coupling system refers to a coupling reagent with or without a coupling additive. In addition, as used herein, a coupling composition refers to a coupling reagent, optionally a coupling additive, and optionally a base.

The coupling of peptide fragments and protected variants thereof as disclosed herein is generally performed with a suitable coupling reagent and/or coupling additive including, but not limited to, one or more of: hydroxybenzotriazole (HOBt), 1-Hydroxy-6-chloro-benzotriazole (6-Cl-HOBt), N,N′-diisopropylcarbodiimide (DIC), O-(Benzotriazol-1-yl)-N,N,N′,N′-tetramethyluronium tetrafluoroborate (TBTU), N-[(dimethylamino)-1H-1,2,3-triazolo[4,5-b]pyridin-1-ylmethylene]-N-methylmethanaminium hexafluorophosphate N-oxide (HATU), N,N,N,N-Tetramethyl-O-(1H-benzotriazol-1-yl) uronium hexafluorophosphate (HBTU), O-(1H-6-Chlorobenzotriazole-1-yl)-1,1,3,3-tetramethyluronium hexafluorophosphate (HCTU), 1,3-dicyclohexylcarbodiimide (DCC), 1-(dimethylaminopropyl)-3-ethylcarbodiimide hydrochloride (EDC HCl), benzotriazol-1-yl-oxy-tris(dimethyl-amino)-phosphonium hexafluorophosphate (BOP), N,N-bis-(2-oxo-3-oxazolidinyl)phosphonic dichloride (BOP—Cl), benzotriazol-1-yloxytri(pyrrolidino)phosphonium hexafluorophosphate (PyBOP), bromotri(pyrrolidino)phosphonium hexafluorophosphate (PyBrOP), chlorotri(pyrrolidino)phosphonium hexafluorophosphate (PyCIOP), [ethyl cyano(hydroxyimino)acetato-O²]tri-1-pyrrolidinylphosphonium hexafluorophosphate (PyOxim), ethyl-2-cyano-2-(hydroxyimino)acetate (Oxyma), O-(6-Chloro-1-hydrocibenzotriazol-1-yl)-1,1,3,3-tetramethyluronium tetrafluoroborate (TCTU), 2,4,5-norbornen-2,3-dicarboximido)-1,1,3,3-tetramethyluronium tetrafluoroborate (TNTU), 2-succinimido-1,1,3,3-tetramethyluronium tetrafluoro borate (TSTU), 1-cyano-2-ethoxy-2-oxoethylidenaminooxy)dimethylamino-morpholino-carbenium hexafluorophosphate (COMU), and 3-(diethoxyphosphoryloxy)-1,2,3-benzotriazin-4 (3H)-one (DEPBT). In some embodiments, propylphosphonic anhydride (T3P) is used as a coupling reagent.

In some embodiments, the coupling reagent comprises a carbodiimide (such as, e.g., DIC, DCC, EDC), an aminium (uronium) salt (e.g., HBTU, TBTU, HATU, TATU, HCTU, TCTU, COMU, TOTU), a phosphonium salt (e.g., PyBOP, PyAOP, PyBroP, BOP), phosphonic acid anhydrides (e.g., T3P), or phosphate-type coupling reagents (e.g., DEPBT, N-diethoxyphosphorylbenzoxazolone (DEPBO), N-(2-oxo-1,3,2-dioxaphosphorinanyl)benzoxazolone (DOPBO), 3-[O-(2-oxo-1,3,2-dioxaphosphorinanyl)oxy]-1,2,3-benzotriazin-4 (3H)-one (DOPBT), diethyl phosphorocyanidate (DEPC), pentafluorophenyl diphenylphosphinate (FDPP), diphenylphosphoryl azide (DPPA)).

In some embodiments, the coupling reagent comprises a carbodiimide (such as, e.g., DIC, DCC, EDC).

In some embodiments, the coupling reagent comprises an aminium (uronium) salt (e.g., HBTU, TBTU, HATU, TATU, HCTU, TCTU, COMU, TOTU) or a phosphonium salt (e.g., PyBOP, PyAOP, PyBroP, BOP).

In some embodiments, the coupling reagent comprises an aminium (uronium) salt (e.g., HBTU, TBTU, HATU, TATU, HCTU, TCTU, COMU, TOTU). In some embodiments, the aminium salt is a HOBt-based uronium salt (e.g., HBTU, TBTU). In some embodiments, the aminium salt is a HOAt-based uronium salt (e.g., HATU, TATU). In some embodiments, the aminium salt is a 6-Cl-HOBt analog (e.g., HCTU, TCTU). In some embodiments, the aminium salt is an oxime-based uronium salt (e.g., an Oxyma-based uronium salt, e.g., COMU) or a fluorinated phenyl uronium salt (e.g., TOTU).

In some embodiments, the coupling reagent comprises a phosphonium salt (e.g., PyBOP, PyAOP, PyBroP, BOP).

In some embodiments, the coupling reagent comprises a phosphonic acid anhydride (T3P).

In some embodiments, the coupling reagent comprises DIC, DCC, EDC.HCl, TBTU, HBTU, HATU, HCTU, TCTU, TNTU, TSTU; BOP, BOP—Cl, PyBOP, PyBrOP, PyCIOP, PyOxim, COMU, DEPBT, or a combination of any of the foregoing. In some embodiments, the coupling reagent comprises DIC, DCC, EDC.HCl, TBTU, HBTU, HATU, HCTU, TCTU, TNTU, TSTU; BOP, BOP—Cl, PyBOP, PyBrOP, PyCIOP, PyOxim, COMU, or DEPBT.

In some embodiments, a coupling additive used in a SPPS coupling step described herein comprises HOBt, 6-Cl-HOBt, Oxyma, or a combination of any of the foregoing. In some embodiments, the coupling additive comprises HOBt, 6-Cl-HOBt, or Oxyma. In some embodiments, the coupling additive is HOBt, 6-Cl-HOBt, or Oxyma.

In some embodiments, a coupling additive used in a SPPS coupling step described herein comprises 1-hydroxy-2-pyridone (HOPO).

In some embodiments, a coupling system used in a SPPS coupling step described herein comprises a carbodiimide (such as, e.g., DIC, DCC, EDC) and a coupling additive selected from hydroxypyridones (e.g., HOPO), N-hydroxybenzotriazoles (e.g., HOBt, 6-Cl-HOBt), azabenzotriazoles (e.g., HOAt), and oxime-based additives (e.g. Oxyma). In some embodiments, a coupling system used in a SPPS coupling step described herein comprises a carbodiimide (such as, e.g., DIC, DCC, EDC) and a coupling additive selected from N-hydroxybenzotriazoles (e.g., HOBt, 6-Cl-HOBt), azabenzotriazoles (e.g., HOAt), and oxime-based additives (e.g. Oxyma). In some embodiments, a coupling system used in a SPPS coupling step described herein comprises a carbodiimide (such as, e.g., DIC, DCC, EDC) and a coupling additive selected from HOPO, HOBt, 6-Cl-HOBt, HOAt, and Oxyma. In some embodiments, a coupling system used in a SPPS coupling step described herein comprises a carbodiimide (such as, e.g., DIC, DCC, EDC) and a coupling additive selected from HOBt, 6-Cl-HOBt, HOAt, and Oxyma. In some embodiments, the coupling system comprises DIC and HOAt. In some embodiments, the coupling system comprises DIC and HOBt. In some embodiments, the coupling system comprises DIC and Oxyma.

In some embodiments, the coupling system comprises DIC and HOPO.

In some embodiments, a coupling system used in a SPPS coupling step described herein comprises an aminium (uronium) salt and a coupling additive selected from N-hydroxybenzotriazoles (e.g., HOBt, 6-Cl-HOBt), azabenzotriazoles (e.g., HOAt), and oxime-based additives (e.g. Oxyma). In some embodiments, the coupling system comprises TBTU and HOBt. In some embodiments, the coupling system comprises HATU and

HOAt. In some embodiments, the coupling system comprises HBTU and HOBt. In some embodiments, the coupling system comprises HBTU and HOAt. In some embodiments, the coupling system comprises HBTU and Oxyma.

In some embodiments, a coupling system used in a SPPS coupling step described herein comprises DIC, TBTU, HATU, PyBOP, PyOxim, HCTU, 6-Cl-HOBt, or a combination of any of the foregoing.

In some embodiments, the coupling reagent comprises DIC, TBTU, HATU, PyBOP, PyOxim, HCTU, 6-Cl-HOBt, or a combination of any of the foregoing. In some embodiments, the coupling reagent comprises DIC, TBTU, HATU, PyBOP, PyOxim, HCTU, or a combination of any of the foregoing.

In some embodiments, a coupling system used in a SPPS coupling step described herein comprises Oxyma, HATU, PyOxim, PyBOP, or TBTU.

In some embodiments, the coupling reagent comprises Oxyma, HATU, PyOxim, PyBOP, or TBTU. In some embodiments, the coupling reagent comprises HATU, PyOxim, PyBOP, or TBTU.

Bases useful in the coupling reactions of the present disclosure include, but are not limited to, “Hunig's base” or N,N′-diisopropylethylamine (DIPEA), 2,4,6-trimethylpyridine (collidine), and triethylamine (TEA). In some embodiments, the base comprises DIPEA, N-methylmorpholine (NMM), N-ethylmorpholine (NEM), TEA, 2,6-lutidine, collidine, 2,2,6,6-tetramethylpiperidine (TMP), or a combination of any of the foregoing. In some embodiments, the base comprises DIPEA, N-methylmorpholine (NMM), N-ethylmorpholine (NEM), TEA, 2,6-lutidine, collidine, or a combination of any of the foregoing. In some embodiments, the base is DIPEA, N-methylmorpholine (NMM), N-ethylmorpholine (NEM), TEA, 2,6-lutidine, collidine, or 2,2,6,6-tetramethylpiperidine (TMP). In some embodiments, the base is DIPEA, N-methylmorpholine (NMM), N-ethylmorpholine (NEM), TEA, 2,6-lutidine, or collidine. In some embodiments, the base is tetramethylethylenediamine (TMEDA).

In some embodiments, the base comprises DIPEA. In some embodiments, the base is DIPEA.

In some embodiments, the base comprises collidine. In some embodiments, the base is collidine.

In some embodiments, the base comprises TEA. In some embodiments, the base is TEA.

In some cases, an additive such as lithium chloride can be used during peptide synthesis. In some embodiments, the additive comprises a chaotropic or solubilizing salt additive (e.g., LiCl, LiBr, NaClO₄, LiClO₄, KSCN).

Particularly useful combinations of coupling reagents, bases, and additives for the coupling reactions of the present disclosure include DIC/Oxyma, PyOxim/collidine, PyBOP/collidine, PyBOP/TEA, TBTU/collidine, HATU/collidine, DIC/LiCl, TBTU/LiCl, and HATU/LiCl.

In some embodiments, a coupling composition used in a SPPS coupling step described herein comprises DIC/Oxyma, PyOxim/collidine, PyBOP/collidine, PyBOP/TEA, TBTU/collidine, HATU/collidine, DIC/LICI, TBTU/LiCl, and HATU/LiCl. In some embodiments, the coupling composition comprises DIC and

Oxyma. In some embodiments, the coupling composition comprises PyOxim and collidine. In some embodiments, the coupling composition comprises PyBOP and collidine. In some embodiments, the coupling composition comprises PyBOP and TEA. In some embodiments, the coupling composition comprises TBTU and collidine. In some embodiments, the coupling composition comprises HATU and collidine. In some embodiments, the coupling composition comprises DIC and LiCl. In some embodiments, the coupling composition comprises TBTU and LiCl. In some embodiments, the coupling composition comprises HATU and LiCl.

In some embodiments, the solid phase coupling conditions of step (A) (i) (a), A (ii), or A (iii) of multi-step SPPS methods described herein (e.g., Embodiments A.90-A. 103) comprise TBTU and collidine in DMSO at room temperature (e.g., a temperature in the range of 20° C. to 25° C.). In some embodiments, the solid phase coupling conditions of step (B) of multi-step SPPS methods described herein (e.g., Embodiments A.90-A. 103) comprise DIC/Oxyma in DMF at a temperature in the range of 45° C. to 55° C.). In some embodiments, the solid phase coupling conditions of step (C) of multi-step SPPS methods described herein (e.g., Embodiments A.90-A. 103) comprise DIC/Oxyma in DMF at room temperature (e.g., a temperature in the range of 20° C. to 25° C.). In some embodiments, the deprotecting of step (D) of multi-step SPPS methods described herein (e.g., Embodiments A.90-A.103) comprises mixing the protected variant of Peptide A (before or after cleaving from solid support) with an aqueous solution comprising trifluoroacetic acid (TFA). In some embodiments, the deprotecting of step (D) of multi-step SPPS methods described herein (e.g., Embodiments A.90-A.103) comprises mixing the protected variant of Peptide A (before or after cleaving from solid support) with an aqueous solution comprising trifluoroacetic acid (TFA) and triisopropyl silane (TIS).

In some embodiments, the solid phase coupling conditions of step (A) (i) (a), A (ii), or A (iii) of multi-step SPPS methods described herein (e.g., Embodiments A.90-A. 103) comprise TBTU and collidine in DMSO at 20° C. In some embodiments, the solid phase coupling conditions of step (B) of multi-step SPPS methods described herein (e.g., Embodiments A.90-A. 103) comprise DIC/Oxyma in DMF at 50° C. In some embodiments, the solid phase coupling conditions of step (C) of multi-step SPPS methods described herein (e.g., Embodiments A.90-A. 103) comprise DIC/Oxyma in DMF at 20° C. In some embodiments, the deprotecting of step (D) of multi-step SPPS methods described herein (e.g., Embodiments A.90-A.103) comprises mixing the protected variant of Peptide A (before or after cleaving from solid support) with an aqueous solution of trifluoroacetic acid (TFA) and triisopropyl silane (TIS).

In some embodiments, an SPPS coupling step described herein is mediated by 1 to 10 equivalents of a coupling reagent. In some embodiments, an SPPS coupling step described herein is mediated by 1 to 10 equivalents of a coupling reagent and 1 to 10 equivalents of a base. In some embodiments, an SPPS coupling step described herein is mediated by 1 to 10 equivalents of a coupling reagent and 1 to 10 equivalents of a coupling additive. In some embodiments, an SPPS coupling step described herein is mediated by 1 to 10 equivalents of a coupling reagent, 1 to 10 equivalents of a base, and 1 to 10 equivalents of a coupling additive.

In some embodiments, an SPPS coupling step described herein is mediated by 1 to 5 equivalents of a coupling reagent. In some embodiments, an SPPS coupling step described herein is mediated by 1 to 5 equivalents of a coupling reagent and 1 to 5 equivalents of a base. In some embodiments, an SPPS coupling step described herein is mediated by 1 to 5 equivalents of a coupling reagent and 1 to 5 equivalents of a coupling additive. In some embodiments, an SPPS coupling step described herein is mediated by 1 to 5 equivalents of a coupling reagent, 1 to 5 equivalents of a base, and 1 to 5 equivalents of a coupling additive.

In some embodiments, an SPPS coupling step described herein is mediated by 2 to 5 equivalents of a coupling reagent. In some embodiments, an SPPS coupling step described herein is mediated by 2 to 5 equivalents of a coupling reagent and 2 to 10 equivalents of a base. In some embodiments, an SPPS coupling step described herein is mediated by 2 to 5 equivalents of a coupling reagent and 2 to 5 equivalents of a coupling additive. In some embodiments, an SPPS coupling step described herein is mediated by 2 to 5 equivalents of a coupling reagent, 2 to 10 equivalents of a base, and 2 to 5 equivalents of a coupling additive.

### Temperature and Reaction Time

The coupling reactions of the present disclosure may be accomplished at any suitable temperature, for example, a temperature in the range of 10° C. to 60° C. Particularly useful reaction temperatures include those in the range of 20° C. to 50° C. For example, suitable reaction temperatures may include 20° C., 25° C., 30° C., 35° C., 40° C., 45° C., and 50° C. Additionally, suitable reaction temperatures may include 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, all in ° C. Certain methods of preparing Peptide A described herein were carried out at a temperature in the range of 20° C. to 50° C.

Additionally, the coupling reactions of the present disclosure may be performed for a time sufficient to achieve a desired conversion rate (e.g., complete or near complete conversion to the desired intermediate or product). In some embodiments, the solid phase peptide coupling reaction is allowed to proceed for 10 minutes to 24 hours. In some embodiments, the solid phase peptide coupling reaction is allowed to proceed for 30 minutes to 24 hours. In some embodiments, the solid phase peptide coupling reaction is allowed to proceed for 1 hour to 24 hours. In some embodiments, the solid phase peptide coupling reaction is allowed to proceed for 2 hours to 24 hours.

In some embodiments, the solid phase peptide coupling conditions for a SPPS coupling step described herein comprise allowing the coupling reaction to proceed for a time in the range of 10 minutes to 24 hours at a temperature in the range of 10° C. to 60° C. In some embodiments, the solid phase peptide coupling conditions comprise allowing the coupling reaction to proceed for a time in the range of 10 minutes to 24 hours at a temperature in the range of 20° C. to 50° C. In some embodiments, the solid phase peptide coupling conditions comprise allowing the coupling reaction to proceed for a time in the range of 10 minutes to 24 hours at a temperature in the range of 20° C. to 25° C.

In some embodiments, the solid phase peptide coupling conditions for a SPPS coupling step described herein comprise allowing the coupling reaction to proceed for a time in the range of 30 minutes to 24 hours at a temperature in the range of 10° C. to 60° C. In some embodiments, the solid phase peptide coupling conditions comprise allowing the coupling reaction to proceed for a time in the range of 30 minutes to 24 hours at a temperature in the range of 20° C. to 50° C. In some embodiments, the solid phase peptide coupling conditions comprise allowing the coupling reaction to proceed for a time in the range of 30 minutes to 24 hours at a temperature in the range of 20° C. to 25° C.

In some embodiments, the solid phase peptide coupling conditions for a SPPS coupling step described herein comprise allowing the coupling reaction to proceed for a time in the range of 1 hour to 24 hours at a temperature in the range of 10° C. to 60° C. In some embodiments, the solid phase peptide coupling conditions comprise allowing the coupling reaction to proceed for a time in the range of 1 hour to 24 hours at a temperature in the range of 20° C. to 50° C. In some embodiments, the solid phase peptide coupling conditions comprise allowing the coupling reaction to proceed for a time in the range of 1 hour to 24 hours at a temperature in the range of 20° C. to 25° C.

In some embodiments, the solid phase peptide coupling conditions for a SPPS coupling step described herein comprise allowing the coupling reaction to proceed for a time in the range of 2 hours to 24 hours at a temperature in the range of 10° C. to 60° C. In some embodiments, the solid phase peptide coupling conditions comprise allowing the coupling reaction to proceed for a time in the range of 2 hours to 24 hours at a temperature in the range of 20° C. to 50° C. In some embodiments, the solid phase peptide coupling conditions comprise allowing the coupling reaction to proceed for a time in the range of 2 hours to 24 hours at a temperature in the range of 20° C. to 25° C.

### Solid Phase Peptide Synthesis Deprotection Conditions

In some embodiments, between each coupling reaction on solid support, the N-terminus of the growing peptide chain is deprotected to expose a free amino group for reaction with an activated carboxyl group at the C-terminus of an incoming amino acid or peptide. In preferred embodiments, deprotection conditions and orthogonal side chain protecting groups are selected to enable the selective removal of the N-terminal protecting group.

In some embodiments, an Fmoc group on an N-terminus of a peptide on solid support as disclosed herein can be removed with an amine base, in preparation for coupling another peptide thereof to the solid support-conjugated peptide. Suitable amine bases include, but are not limited to, piperidine, piperazine, N-methyl morpholine, diethylamine, triethylamine, 1,8-diazabicyclo[5.4.0]undec-7-ene (DBU), and mixtures of any of the foregoing. In some embodiments, the amine base is piperidine. The removal is conducted in a solvent. In some embodiments, the solvent is N, N-dimethylformamide (DMF), methylene chloride, tetrahydrofuran, N-methyl pyrrolidine, or mixtures of any of the foregoing. In some embodiments, the amine base is piperidine in DMF, or 4-methylpiperidine.

In some embodiments, amide moieties may be protected with Boc and Trt groups, which may be removed with TFA.

For example, in some embodiments, a protected peptide with a Boc-protected N-terminus may be selectively deprotected by contacting the protected peptide with an acid (preferably an acid selected from TFA, HCl, and HBr) to selectively remove the Boc protecting group without removing orthogonal side chain protecting groups. In some embodiments, the protected peptide is contacted with TFA (e.g., neat TFA or TFA in DCM, e.g., 20-50% (v/v) TFA in DCM) to remove the Boc protecting group. In some embodiments, the protected peptide is contacted with HCl (e.g., HCl in DCM, DCE, toluene, or dioxane, e.g., 3-4 M HCl in DCM or dioxane) to remove the Boc protecting group.

In certain embodiments, selective deprotection of a peptide with a Boc-protected N-terminus comprises contacting the protected peptide with an acid (preferably an acid selected from TFA, HCl, and HBr) in the presence of a scavenger. In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some preferred embodiments, the scavenger is a hydrosilane. In some particularly preferred embodiments, the scavenger is TIS. In some embodiments, the protected peptide is contacted with TFA (e.g., neat TFA or TFA in DCM, e.g., 20-50% (v/v) TFA in DCM) in the presence of a scavenger (preferably TIS) to remove the Boc protecting group. In some embodiments, the protected peptide is contacted with HCl (e.g., HCl in DCM, DCE, toluene, or dioxane, e.g., 3-4 M HCl in DCM or dioxane) in the presence of a scavenger to remove the Boc protecting group.

In some embodiments, a protected peptide with a Trt-protected N-terminus can be selectively deprotected by contacting the protected peptide with a Brønsted acid or a Lewis acid (preferably TFA, HCl, or dichloroacetic acid, most preferably TFA), optionally in the presence of a scavenger (preferably TIS), to selectively remove the trityl protecting group at the N-terminus without removing orthogonal side chain protecting groups. In some embodiments, the protected peptide is contacted with a Brønsted acid (preferably a dilute Brønsted acid). In some embodiments, the Brønsted acid is selected from TFA, HCl, and dichloroacetic acid (preferably dilute TFA, HCl, or dichloroacetic acid). In other embodiments, the protected peptide is contacted with a Lewis acid (preferably a dilute Lewis acid). the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some embodiments, the scavenger is a hydrosilane. In some embodiments, the scavenger is TIS.

In some cases, deprotecting can comprise exposure to hydrazine. Illustratively, in certain embodiments in which a Dde or ivDde protecting group is used to protected an amino group (e.g., an N-terminal amino group or a side chain amine (e.g., an s-amino group of a lysine residue), selective deprotection can comprise contacting the protected peptide with an α-nucleophile such as hydrazine to remove the Dde or ivDde protecting group.

Cleavage from Solid Support

In some embodiments of solid phase peptide synthesis methods described herein, an assembled peptide may be removed from solid support by cleaving a linker that covalently attaches the peptide to the solid support. In some embodiments, the peptide is covalently linked to the solid support (e.g., a Rink amide, Wang, Sieber amide, or 2-CTC resin) via an acid-labile linker, and the peptide is removed from the solid support by contacting the peptide-solid support conjugate with an acid (e.g., TFA, optionally in the presence of a scavenger). In other embodiments, the peptide is covalently linked to the solid support (e.g., a Merrifield or MBHA resin) via a benzyl-type linker, and the peptide is removed from the solid support by contacting the peptide-solid support conjugate with HF, TFMSA, or TMSOTf (preferably anhydrous THF).

Illustratively, in some embodiments in which an acid-labile linker is used to covalently link Peptide A or a protected thereof to a solid support (e.g., a Rink amide, Wang, Sieber amide, or 2-CTC resin), Peptide A or a protected variant thereof can generally be cleaved from the solid support under acidic conditions. Such acidic conditions generally also globally deprotect all remaining acid-labile protecting groups (e.g., BOC, tBu, Trt and Pbf). The cleavage of the peptide from the solid support involves treating the protected peptide, anchored to the solid support, with an acid in the presence of at least one scavenger. A particularly suitable acid is TFA. Suitable scavengers include, but are not limited to, triisopropyl silane (TIPS), phenol, thioanisole, water, ethanedithiol (EDT), and mixtures thereof. In some embodiments, the cleavage solution is TFA/TIPS in water in a volume ratio of 95/2.5/2.5.

In alternative embodiments, including certain embodiments in which Boc groups are used to protect N-termini and a benzyl-type linker is used to covalently link peptides to solid supports (e.g., Merrifield or MBHA resins), a peptide may be cleaved from a solid support using HF, TFMSA, or TMSOTf (preferably anhydrous HF), typically after the peptide on solid support is contacted with TFA to remove an N-terminal Boc protecting group. Exposure to HF, TFMSA, or TMSOTf during the cleavage generally also removes benzyl-type side chain protecting groups, enabling simultaneous cleavage and global deprotection.

### Liquid Phase Peptide Synthesis Coupling Conditions

In liquid phase peptide synthesis, which is also referred to as solution phase peptide synthesis herein, one or more peptide coupling reactions are performed in solution rather than on solid support. Two non-limiting examples of liquid phase peptide synthesis approaches are classical solution-phase synthesis and tag-assisted liquid-phase synthesis. In classical solution-phase synthesis, a peptide chain can be iteratively elongated through coupling and deprotection reactions conducted in solution, with intermediate peptides generally purified after each step using methods such as chromatography or crystallization. Alternatively, tag-assisted liquid-phase synthesis uses a soluble tag covalently linked to the growing peptide chain (e.g., a PEG, fluorous, or hydrophobic tag typically attached to the peptide's C-terminus) to improve solubility and simplify the purification of intermediates by, for example, facilitating purification by selective precipitation, extraction, or phase switching. For example, a benzyl alcohol (3,4,5-tri(octadecyloxy)benzyl alcohol) group with the following structure,

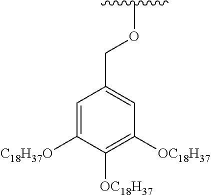

which may be referred to as a “C18 tag” or “C18 TAG” herein, can be coupled to an amino acid or peptide at the C-terminus to mask the amino acid or peptide's C-terminal carboxyl group and enable tag-assisted liquid phase peptide synthesis. Both classical solution-phase synthesis and tag-assisted liquid phase synthesis strategies may be employed in certain liquid phase peptide synthesis approaches described herein.

In liquid-phase peptide synthesis, one or more peptide fragments and one or more reagents (e.g., a coupling reagent, a base, a coupling additive, a deprotecting reagent, etc.) may be combined and solubilized within at least one reaction vessel. Suitable reaction vessels include, but are not limited to, a vial, an EASYMAX reactor, and a CHEMGLASS reactor. A coupling reaction between two peptide fragments may occur in the same reaction vessel used for solubilizing one or both peptide fragments, or alternatively, in a separate reaction vessel from the reaction vessel(s) used for solubilizing one or both peptide fragments. In some embodiments, the reaction vessel is a vial, an EASYMAX reactor, or a CHEMGLASS reactor. In some embodiments, the reaction vessel is a vial. In some embodiments, the reaction vessel is an EASYMAX reactor. In some embodiments, the reaction vessel is a CHEMGLASS reactor.

In some embodiments, a peptide fragment (e.g., a protected peptide fragment described herein) can optionally be milled to reduce its particle size prior to coupling. Milling techniques that may be used include, but are not limited to, slurry milling, conical milling, and pin-milling. In some embodiments, the milling reduces the particle size to an average of less than 60 microns. In some embodiments, the particle size may be reduced to 60, 55, 50, 45, 40, 35, 30, 25 or 20 microns. In some embodiments, the milling may be performed at a temperature of 25, 30, 35, 40, 45, 50, 55, 60, or 65° C.

In preferred embodiments of liquid phase peptide synthesis methods described herein, liquid phase peptide coupling conditions (e.g., identity and amount(s) of coupling reagent, base (if any), and coupling additive (if any); solvent(s); temperature(s); and order of addition, including choice of preactivation vs. in situ activation of a C-terminal carboxyl group) can be selected to limit the occurrence of issues that may arise during peptide coupling (e.g., racemization at the α-stereocenter, guanidination/alkylation of amines, N-acylurea formation, and self-association/aggregation-driven side reaction). Additionally, in preferred embodiments, a peptide is assembled using side chain protecting groups that are compatible with the selected coupling and selective N-terminal protecting group deprotection conditions.

### Solvents

The liquid phase peptide synthesis methods of the present disclosure can be carried out in one or more reaction vessels in a variety of organic solvents and solvent systems. Preferred solvents are polar, aprotic solvents, including combinations of such solvents. Non-limiting example solvents include DMF, DMAc, DMSO, water, acetonitrile, NBP, EtOAc, THF, 2-methyltetrahydrofuran (2-MeTHF), N,N-dimethylacetamide (DMAc), and combinations of any of the foregoing. In some embodiments, an LPPS coupling step described herein can be performed in THF, DMF, water, NBP, DMSO, EtOAc, 2-MeTHF, and combinations of any of the foregoing.

In some embodiments, an LPPS coupling step described herein is performed in acetonitrile. In some embodiments, an LPPS coupling step described herein is performed in DMF. In some embodiments, an LPPS coupling step described herein is performed in NBP. In some embodiments, an LPPS coupling step described herein is performed in DMSO. In some embodiments, an LPPS coupling step described herein is performed in EtOAc. In some embodiments, an LPPS coupling step described herein is performed in THF. In some embodiments, an LPPS coupling step described herein is performed in 2-MeTHF.

Particularly useful solvents in the methods of the present disclosure include acetonitrile, THF, and DMSO. For example, in some preferred embodiments, an LPPS coupling step described herein is performed in a solvent system comprising DMSO and acetonitrile. In other preferred embodiments, an LPPS coupling step described herein is performed in a solvent system comprising DMSO and THF.

In alternative embodiments, an LPPS coupling step described herein is performed in a solvent system comprising an organic solvent and an alcohol co-solvent. In some alternative embodiments, an LPPS coupling step described herein is performed in a solvent system comprising DMSO and MeOH.

In alternative embodiments, an LPPS coupling step described herein is performed in a solvent system comprising an organic solvent and water. In some alternative embodiments, an LPPS coupling step described herein is performed in a solvent system comprising THF and water.

### Coupling Reagents, Bases, and Additives

In liquid-phase peptide synthesis (LPPS), a coupling reagent is typically used to activate the C-terminal carboxyl group of a protected amino acid or peptide fragment (e.g., an amino acid or peptide fragment with an Fmoc- or Boc-protected N-terminus and orthogonal side chain protecting groups) to form an activated/reactive intermediate that facilitates amide bond formation. Additionally, in some embodiments of methods described herein, activation of the C-terminal carboxyl group can be accomplished through preactivation, wherein the carboxyl group is contacted with a coupling reagent to form an activated intermediate before being in the presence of the coupling partner, or through activation in situ, wherein the activated intermediate is generated directly in the presence of the coupling partner. In certain embodiments, a peptide or protected variant thereof may initially be present in the form of a carboxylate salt; in such cases, the peptide or protected variant is converted to a free acid and then activated. A non-nucleophilic tertiary base (e.g., DIPEA, NMM, 2,6-lutidine, or collidine) can be used in conjunction with a coupling reagent to deprotonate the N-terminal amino group (or liberate it when the amine coupling partner is initially present in the form of an acid-addition salt) of the coupling partner (e.g., an amino acid or peptide fragment with a free N-terminal amino group and protected side chains), maintain nucleophilicity, and neutralize acids generated during activation. In addition, a coupling additive (e.g., HOBt, 6-Cl-HOBt, HOAt, or Oxyma) can be used to accelerate coupling and further suppress racemization.

The coupling of peptide fragments and protected variants thereof as disclosed herein is generally performed with a suitable coupling reagent and, where desired, a coupling additive including, but not limited to, one or more of: hydroxybenzotriazole (HOBt), 1-Hydroxy-6-chloro-benzotriazole (6-Cl-HOBt), N,N′-diisopropylcarbodiimide (DIC), O-(Benzotriazol-1-yl)-N,N,N′,N′-tetramethyluronium tetrafluoroborate (TBTU), N-[(dimethylamino)-1H-1,2,3-triazolo[4,5-b]pyridin-1-ylmethylene]-N-methylmethanaminium hexafluorophosphate N-oxide (HATU), N,N,N,N-Tetramethyl-O-(1H-benzotriazol-1-yl) uronium hexafluorophosphate (HBTU), O-(1H-6-Chlorobenzotriazole-1-yl)-1,1,3,3-tetramethyluronium hexafluorophosphate (HCTU), 1,3-dicyclohexylcarbodiimide (DCC), 1-(dimethylaminopropyl)-3-ethylcarbodiimide hydrochloride (EDC HCl), benzotriazol-1-yl-oxy-tris(dimethyl-amino)-phosphonium hexafluorophosphate (BOP), N,N-bis-(2-oxo-3-oxazolidinyl)phosphonic dichloride (BOP—Cl), benzotriazol-1-yloxytri(pyrrolidino)phosphonium hexafluorophosphate (PyBOP), bromotri(pyrrolidino)phosphonium hexafluorophosphate (PyBrOP), chlorotri(pyrrolidino)phosphonium hexafluorophosphate (PyCIOP), [ethyl cyano(hydroxyimino)acetato-O²]tri-1-pyrrolidinylphosphonium hexafluorophosphate (PyOxim), ethyl-2-cyano-2-(hydroxyimino)acetate (Oxyma), O-(6-Chloro-1-hydrocibenzotriazol-1-yl)-1,1,3,3-tetramethyluronium tetrafluoroborate (TCTU), 2,4,5-norbornen-2,3-dicarboximido)-1,1,3,3-tetramethyluronium tetrafluoroborate (TNTU), 2-succinimido-1,1,3,3-tetramethyluronium tetrafluoro borate (TSTU), 1-cyano-2-ethoxy-2-oxoethylidenaminooxy)dimethylamino-morpholino-carbenium hexafluorophosphate (COMU), and 3-(diethoxyphosphoryloxy)-1,2,3-benzotriazin-4 (3H)-one (DEPBT). In some embodiments, propylphosphonic anhydride (T3P) is used as a coupling reagent.

In some embodiments, an LPPS coupling step employs a coupling additive selected from HOBt, 6-Cl-HOBt, HOAt, Oxyma, and combinations of any of the foregoing. In some embodiments, an LPPS coupling step employs 4-(dimethylamino)pyridine (DMAP) as a coupling additive. In some embodiments, an LPPS coupling step employs 1-hydroxy-2-pyridone (HOPO) as a coupling additive.

In some embodiments, an LPPS coupling step employs a coupling reagent selected from carbodiimides (e.g., DIC, DCC, EDC.HCl), aminium/uronium salts (e.g., HBTU, TBTU, HATU, HCTU, TCTU, TNTU, TSTU, COMU), phosphonium salts (e.g., PyBOP, PyBrOP, PyCIOP, BOP, BOP—Cl, PyOxim), phosphonic acid anhydrides (e.g., T3P), and phosphate-type coupling reagents (e.g., DEPBT, N-diethoxyphosphorylbenzoxazolone (DEPBO), N-(2-oxo-1,3,2-dioxaphosphorinanyl)benzoxazolone (DOPBO), 3-[O-(2-oxo-1,3,2-dioxaphosphorinanyl)oxy]-1,2,3-benzotriazin-4 (3H)-one (DOPBT), diethyl phosphorocyanidate (DEPC), pentafluorophenyl diphenylphosphinate (FDPP), diphenylphosphoryl azide (DPPA)). In some embodiments, the coupling reagent comprises a carbodiimide (e.g., DIC, DCC, EDC.HCl), an aminium (uronium) salt (e.g., HBTU, TBTU, HATU, HCTU, TCTU, COMU, TSTU, TNTU), or a phosphonium salt (e.g., PyBOP, PyAOP, PyBrOP, PyCIOP, BOP, BOP—Cl, PyOxim).

In some embodiments, the coupling reagent comprises a carbodiimide (e.g., DIC, DCC, EDC.HCl).

In some embodiments, the coupling reagent comprises an aminium (uronium) salt (e.g., HBTU, TBTU, HATU, HCTU, TCTU, COMU, TSTU, TNTU).

In some embodiments, the coupling reagent comprises a phosphonium salt (e.g., PyBOP, PyAOP, PyBrOP, PyCIOP, BOP, BOP—Cl, PyOxim).

In some embodiments, the coupling reagent comprises HATU, PyOxim, PyBOP, or TBTU.

In some embodiments, an LPPS coupling step employs a coupling reagent selected from DIC, DCC, EDC.HCl, TBTU, HBTU, HATU, HCTU, TCTU, TNTU, TSTU, BOP, BOP—Cl, PyBOP, PyBrOP, PyCIOP, PyOxim, COMU, DEPBT, or a combination of any of the foregoing.

In some embodiments, a coupling system used in an LPPS coupling step described herein comprises a carbodiimide (e.g., DIC, DCC, EDC.HCl) and a coupling additive selected from HOBt, 6-Cl-HOBt, HOAt, or Oxyma. In some embodiments, the coupling system comprises DIC and HOAt. In some embodiments, the coupling system comprises DIC and HOBt. In some embodiments, the coupling system comprises DIC and Oxyma.

In some embodiments, a coupling system used in an LPPS coupling step described herein comprises a carbodiimide (e.g., DIC, DCC, EDC.HCl) and 1-hydroxy-2-pyridone (HOPO) as a coupling additive.

In some embodiments, a coupling system used in an LPPS coupling step described herein comprises an aminium (uronium) salt or phosphonium salt together with an optional coupling additive selected from HOBt, 6-Cl-HOBt, HOAt, and Oxyma. In some embodiments, the coupling system comprises HBTU and HOBt. In some embodiments, the coupling system comprises HBTU and HOAt. In some embodiments, the coupling system comprises HBTU and Oxyma. In some embodiments, the coupling system comprises HATU and HOAt. In some embodiments, the coupling system comprises TBTU and HOBt.

In some embodiments, a coupling system used in an LPPS coupling step described herein comprises a coupling reagent selected from HATU, PyOxim, PyBOP, and TBTU and optionally a coupling additive selected from HOBt, HOAt, and Oxyma.

Bases useful in the coupling reactions of the present disclosure include, but are not limited to, “Hünig's base” or N,N′-diisopropylethylamine (DIPEA), 2,4,6-trimethylpyridine (collidine), and triethylamine (TEA). In some embodiments, the base comprises DIPEA, N-methylmorpholine (NMM), N-ethylmorpholine (NEM), TEA, 2,6-lutidine, collidine, 2,2,6,6-tetramethylpiperidine (TMP), or a combination of any of the foregoing. In some embodiments, the base comprises DIPEA, N-methylmorpholine (NMM), N-ethylmorpholine (NEM), TEA, 2,6-lutidine, collidine, or a combination of any of the foregoing. In some embodiments, the base is DIPEA, N-methylmorpholine (NMM), N-ethylmorpholine (NEM), TEA, 2,6-lutidine, collidine, or 2,2,6,6-tetramethylpiperidine (TMP). In some embodiments, the base is DIPEA, N-methylmorpholine (NMM), N-ethylmorpholine (NEM), TEA, 2,6-lutidine, or collidine. In some embodiments, the base is tetramethylethylenediamine (TMEDA).

In some embodiments, the base comprises DIPEA. In some embodiments, the base is DIPEA.

In some embodiments, the base comprises collidine. In some embodiments, the base is collidine.

In some embodiments, the base comprises TEA. In some embodiments, the base is TEA.

In some cases, an additive such as lithium chloride can be used during peptide synthesis. In some embodiments, the additive comprises a chaotropic or solubilizing salt additive (e.g., LiCl, LiBr, NaClO₄, LiClO₄, KSCN).

Particularly useful combinations of coupling reagents, bases, and additives for the coupling reactions of the present disclosure include DIC/Oxyma, PyOxim/collidine, PyBOP/collidine, PyBOP/TEA, TBTU/collidine, HATU/collidine, DIC/LiCl, TBTU/LiCl, and HATU/LiCl. Additional coupling compositions that can be used in an LPPS coupling step described herein include, but are not limited to, DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, and Oxyma/PyOxim/collidine. In addition, DIC/HOPO can be used as a coupling composition in an LPPS coupling step described herein.

In some embodiments, a coupling composition used in an LPPS coupling step described herein comprises DIC/Oxyma, PyOxim/collidine, PyBOP/collidine, PyBOP/TEA, TBTU/collidine, HATU/collidine, DIC/LiCl, TBTU/LiCl, or HATU/LiCl. In some embodiments, the coupling composition comprises DIC and Oxyma. In some embodiments, the coupling composition comprises PyOxim and collidine. In some embodiments, the coupling composition comprises PyBOP and collidine. In some embodiments, the coupling composition comprises PyBOP and TEA. In some embodiments, the coupling composition comprises TBTU and collidine. In some embodiments, the coupling composition comprises HATU and collidine. In some embodiments, the coupling composition comprises DIC and LiCl. In some embodiments, the coupling composition comprises TBTU and LiCl. In some embodiments, the coupling composition comprises HATU and LiCl.

In some embodiments, the coupling composition comprises T3P and Oxyma. In some embodiments, the coupling composition comprises T3P, Oxyma, and collidine. In some embodiments, the coupling composition comprises HCTU and collidine. In some embodiments, the coupling composition comprises HATU and collidine. In some embodiments, the coupling composition comprises HBTU and collidine. In some embodiments, the coupling composition comprises DMAP and EDC. In some embodiments, the coupling composition comprises Oxyma and PyOxim. In some embodiments, the coupling composition comprises Oxyma, PyOxim, and collidine.

In some embodiments, a coupling composition used in an LPPS coupling step comprises PyBOP and collidine. In some embodiments, the coupling composition comprises PyBOP and collidine in THF and optionally DMSO. In some embodiments, the coupling composition comprises PyBOP and collidine in THF and DMSO.

In some embodiments, a coupling composition used in an LPPS coupling step comprises PyBOP and TEA. In some embodiments, the coupling composition comprises PyBOP and TEA in DMSO.

In some embodiments, a coupling composition used in an LPPS coupling step comprises EDC, HOBt, and collidine. In some embodiments, the coupling composition comprises EDC, HOBt, and collidine in THF and water.

In some embodiments, a coupling composition used in an LPPS coupling step comprises TBTU and collidine. In some embodiments, the coupling composition comprises TBTU and collidine in DMSO and optionally acetonitrile. In some embodiments, the coupling composition comprises TBTU and collidine in DMSO. In some embodiments, the coupling composition comprises TBTU and collidine in acetonitrile and DMSO.

In some embodiments, a coupling composition used in an LPPS coupling step comprises HCTU, 6-Cl-HOBt, and collidine. In some embodiments, the coupling composition comprises HCTU, 6-Cl-HOBt, and collidine in acetonitrile.

In some embodiments, a coupling composition used in an LPPS coupling step comprises DIC and Oxyma. In some embodiments, the coupling composition comprises DIC and Oxyma in DMSO. In some embodiments, the coupling composition comprises DIC and Oxyma in DMF.

In some embodiments, a coupling composition used in an LPPS coupling step comprises DIC and HOPO in DMSO and optionally MeOH. In some embodiments, a coupling composition used in an LPPS coupling step comprises DIC and HOPO in 4-1:DMSO:MeOH (e.g., 1:1 DMSO:MeOH).

In some embodiments, an LPPS coupling step described herein is mediated by 1 to 10 equivalents of a coupling reagent. In some embodiments, an LPPS coupling step described herein is mediated by 1 to 10 equivalents of a coupling reagent and 1 to 10 equivalents of a base. In some embodiments, an LPPS coupling step described herein is mediated by 1 to 10 equivalents of a coupling reagent and 1 to 10 equivalents of a coupling additive. In some embodiments, an LPPS coupling step described herein is mediated by 1 to 10 equivalents of a coupling reagent, 1 to 10 equivalents of a base, and 1 to 10 equivalents of a coupling additive.

In some embodiments, an LPPS coupling step described herein is mediated by 1 to 5 equivalents of a coupling reagent. In some embodiments, an LPPS coupling step described herein is mediated by 1 to 5 equivalents of a coupling reagent and 1 to 5 equivalents of a base. In some embodiments, an LPPS coupling step described herein is mediated by 1 to 5 equivalents of a coupling reagent and 1 to 5 equivalents of a coupling additive. In some embodiments, an LPPS coupling step described herein is mediated by 1 to 5 equivalents of a coupling reagent, 1 to 5 equivalents of a base, and 1 to 5 equivalents of a coupling additive.

In some embodiments, an LPPS coupling step described herein is mediated by 2 to 5 equivalents of a coupling reagent. In some embodiments, an LPPS coupling step described herein is mediated by 2 to 5 equivalents of a coupling reagent and 2 to 10 equivalents of a base. In some embodiments, an LPPS coupling step described herein is mediated by 2 to 5 equivalents of a coupling reagent and 2 to 5 equivalents of a coupling additive. In some embodiments, an LPPS coupling step described herein is mediated by 2 to 5 equivalents of a coupling reagent, 2 to 10 equivalents of a base, and 2 to 5 equivalents of a coupling additive.

### Temperature and Reaction Time

The liquid phase peptide coupling reactions and deprotection reactions of the present disclosure may be accomplished at any suitable temperature, for example, a temperature in the range of 0° C. to 60° C., such as, e.g., a temperature in the range of 10° C. to 60° C. Particularly useful reaction temperatures include those in the range of 20° C. to 50° C. For example, suitable reaction temperatures may include 20° C., 25° C., 30° C., 35° C., 40° C., 45° C., and 50° C. Additionally, suitable reaction temperatures may include 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, all in ° C. Certain methods of preparing Peptide A described herein were carried out at a temperature in the range of 20° C. to 50° C.

In some embodiments, the reaction temperature for an LPPS coupling step described herein is room temperature (e.g., a temperature in the range of 20° C. to 25° C.).

In some embodiments, the reaction temperature for an LPPS coupling step described herein is room temperature (e.g., a temperature in the range of 30° C. to 50° C., e.g., a temperature in the range of 35° C. to 45° C.).

The coupling reactions and deprotection reactions of the present disclosure may take place in about 1 to about 72 hours, such as, e.g., a reaction time in the range of 1 hour to 72 hours. In some embodiments, the coupling reaction is allowed to proceed for a time in the range of 6 hours to 24 hours. Suitable reaction times may include, but are not limited to, 6, 7, 8, 9, 10, 11, 12, 13, 14, 15, 16, 17, 18, 19, 20, 21, 22, 23 or 24 hours.

In some embodiments, an LPPS coupling step described herein is conducted at room temperature (e.g., a temperature in the range of 20° C. to 25° C.) for a time in the range of 1 hour to 72 hours. In some embodiments, an LPPS coupling step described herein is conducted at room temperature (e.g., a temperature in the range of 20° C. to 25° C.) for a time in the range of 6 hours to 72 hours. In some embodiments, an LPPS coupling step described herein is conducted at room temperature (e.g., a temperature in the range of 20° C. to 25° C.) for a time in the range of 6 hours to 24 hours.

In some embodiments, an LPPS coupling step described herein is conducted at a temperature in the range of 20° C. to 50° C. (e.g., a temperature in the range of 30° C. to 50° C., e.g., a temperature in the range of 35° C. to 45° C.) for a time in the range of 1 hour to 72 hours. In some embodiments, an LPPS coupling step described herein is conducted at a temperature in the range of 20° C. to 50° C. (e.g., a temperature in the range of 30° C. to 50° C., e.g., a temperature in the range of 35° C. to 45° C.) for a time in the range of 6 hours to 72 hours. In some embodiments, an LPPS coupling step described herein is conducted a temperature in the range of 20° C. to 50° C. (e.g., a temperature in the range of 30° C. to 50° C., e.g., a temperature in the range of 35° C. to 45° C.) for a time in the range of 6 hours to 24 hours.

### Liquid Phase Peptide Synthesis Deprotection Conditions

In some embodiments of liquid phase peptide synthesis methods described herein, a protected peptide or amino acid may be selectively deprotected to expose an N-terminal amino group for reaction with an activated carboxyl group at the C-terminus of another amino acid or peptide. In preferred embodiments, deprotection conditions and orthogonal side chain protecting groups are selected to enable the selective removal of the N-terminal protecting group.

In some embodiments, a protected peptide may be selectively deprotected by contacting the peptide with an amine base to selectively remove the Fmoc protecting group without removing orthogonal side chain protecting groups. Suitable amine bases include, but are not limited to, piperidine, piperazine, N-methyl morpholine, diethylamine, triethylamine, 1,8-diazabicyclo[5.4.0]undec-7-ene (DBU), and mixtures of any of the foregoing. In some embodiments, the amine base is piperidine. In some embodiments, the protected peptide is contacted with the amine base in a solvent selected from N,N-dimethylformamide (DMF), methylene chloride, tetrahydrofuran, N-methylpyrrolidine, and combinations of any of the foregoing. In some embodiments, the protected peptide is contacted with piperidine (preferably piperidine in DMF). In some embodiments, the protected peptide is contacted with 4-methylpiperidine.

In some embodiments, a protected peptide with a Boc-protected N-terminus may be selectively deprotected by contacting the protected peptide with an acid (preferably an acid selected from TFA, HCl, and HBr) to selectively remove the Boc protecting group without removing orthogonal side chain protecting groups. In some embodiments, the protected peptide is contacted with TFA (e.g., neat TFA or TFA in DCM, e.g., 20-50% (v/v) TFA in DCM) to remove the Boc protecting group. In some embodiments, the protected peptide is contacted with HCl (e.g., HCl in DCM, DCE, toluene, or dioxane, e.g., 3-4 M HCl in DCM or dioxane) to remove the Boc protecting group.

In certain embodiments, selective deprotection of a peptide with a Boc-protected N-terminus comprises contacting the protected peptide with an acid (preferably an acid selected from TFA, HCl, and HBr) in the presence of a scavenger. In some embodiments, the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some preferred embodiments, the scavenger is a hydrosilane. In some particularly preferred embodiments, the scavenger is TIS. In some embodiments, the protected peptide is contacted with TFA (e.g., neat TFA or TFA in DCM, e.g., 20-50% (v/v) TFA in DCM) in the presence of a scavenger (preferably TIS) to remove the Boc protecting group. In some embodiments, the protected peptide is contacted with HCl (e.g., HCl in DCM, DCE, toluene, or dioxane, e.g., 3-4 M HCl in DCM or dioxane) in the presence of a scavenger to remove the Boc protecting group.

In some embodiments, a protected peptide with a Trt-protected N-terminus can be selectively deprotected by contacting the protected peptide with a Brønsted acid or a Lewis acid (preferably TFA, HCl, or dichloroacetic acid, most preferably TFA), optionally in the presence of a scavenger (preferably TIS), to selectively remove the trityl protecting group at the N-terminus without removing orthogonal side chain protecting groups. In some embodiments, the protected peptide is contacted with a Brønsted acid (preferably a dilute Brønsted acid). In some embodiments, the Brønsted acid is selected from TFA, HCl, and dichloroacetic acid (preferably dilute TFA, HCl, or dichloroacetic acid). In other embodiments, the protected peptide is contacted with a Lewis acid (preferably a dilute Lewis acid). the scavenger is selected from hydrosilanes (e.g., TIS, triethylsilane (TES)), phenols (e.g., phenol, anisole, m-cresol), thioethers (e.g., thioanisole, dimethyl sulfide), thiols/dithiols (e.g., EDT, DTT, 1-dodecanethiol), water, indole, and combinations of any of the foregoing. In some embodiments, the scavenger is a hydrosilane. In some embodiments, the scavenger is TIS.

In some embodiments in which a Dde or ivDde protecting group is used to protected an amino group (e.g., an N-terminal amino group or a side chain amine (e.g., an ¿-amino group of a lysine residue), selective deprotection can comprise contacting the protected peptide with an α-nucleophile (e.g., hydrazine or hydroxylamine, preferably hydrazine) to remove the Dde or ivDde protecting group.

### Liquid Phase Peptide Synthesis Isolation Conditions

In some embodiments, peptides and protected variants thereof produced by a liquid phase peptide synthesis method described herein can be isolated by filtration and/or precipitation. In certain embodiments, the reaction mixture can be poured into water to precipitate the desired peptide. In some embodiments, the precipitation is performed at a temperature in the range of 0° C. to 25° C.

In some embodiments, filtration can be performed subsequent to precipitation. In some embodiments, the filter is a 10 micron polytetrafluoroethylene filter or a 5-10 micron nylon filter. The solid can be washed one or more times with one or more solvents such as acetonitrile, DMSO, DMF, water, NBP, EtOAc, THF, or 2-MeTHF. The solid can then be washed one or more times with one or more solvents, such as, e.g., MTBE, MeCN, THF, diethyl ether, dioxane, dimethoxyethane, diglyme, 2-butoxyethanol, and combinations of any of the foregoing. In some embodiments, the solid may be washed one or more times with a first solvent or solvents, then washed one or more times with a second solvent or solvents, and then washed one or more times with a third solvent or solvents. In some embodiments, the wash solvent is a 4:1 combination of acetonitrile and DMSO, or a 2:1:1 combination of water, acetonitrile and THF, or a 4:1 combination of THF and DMSO. In other embodiments, the wash solvent is a 3:2 combination of THF and water. In some embodiments, the solid can be dried under vacuum in a nitrogen atmosphere after washing.

The following examples are given for the purpose of illustrating various embodiments of the disclosure and are not meant to limit the present disclosure in any fashion. One skilled in the art will appreciate readily that the present disclosure is well-adapted to carry out the objects and obtain the ends and advantages mentioned, as well as those objects, ends, and advantages inherent herein. Changes therein and other uses which are encompassed within the spirit of the disclosure as defined by the scope of the claims will occur to those skilled in the art.

### EXAMPLES

Provided in this section are descriptions of the general analytical and purification methods used to prepare the specific examples provided herein.

The following examples are provided for illustration and are not intended to limit the scope of the invention.

List of Abbreviations

| | |
|---|---|
| 2-MeTHF | 2-methyltetrahydrofuran |
| 6-Cl-HOBt | 6-chloro-1-hydroxybenzotriazole |
| ACN, MeCN | acetonitrile |
| BOC or Boc | tert-butyloxycarbonyl |
| BrCH₂CO | bromoacetyl |
| (BrCH₂CO)₂O | bromoacetic anhydride |
| BrCH₂CO₂H | bromoacetic acid |
| CPME | cyclopentyl methyl ether |
| DBU | 1,8-diazabicyclo[5.4.0]undec-7-ene |
| DCC | 1,3-dicyclohexylcarbodiimide |
| DIC | N,N-diisopropylcarbodiimide |
| DMAc | N,N-dimethylacetamide |
| DMF | N,N-dimethylformamide |
| DMI | 1,3-dimethyl-2-imidazolidinone |
| DIPEA, DIEA | N-ethyl diisopropylamine (Hünig's base) |
| DMSO | dimethylsulfoxide |
| DR, D.R., dr, d.r. | diastereomeric ratio |
| EDC | 1-ethyl-3-(3-dimethylaminopropyl)carbodiimide |
| EOR | end of reaction |
| eq or eq. or equiv. | equivalent |
| EtOAc | ethyl acetate |
| Fmoc | 9-fluorenylmethoxycarbonyl |
| h | hour(s) |
| HATU | 1-[bis(dimethylamino)methylene]-1H-1,2,3-triazolo[4,5-b]pyridinium 3-oxid |
| | hexafluorophosphate (uronium coupling reagent) |
| HCTU | 2-(6-chloro-1H-benzotriazole-1-yl-1, 13,3-tetramethylaminium |
| | hexafluorophosphate |
| HOBt | 1-hydroxybenzotriazole |
| HOPO | 2-hydroxypyridine N-oxide |
| HPLC | high-performance liquid chromatography |
| IPA | isopropyl alcohol |
| IPC | in-process control |
| ivDde | N-ε-1-(4,4-dimethyl-2,6-dioxocyclohex-1-ylidene)-3-methylbutyl amine |
| LC/MS | liquid chromatography/mass spectrometry |
| LCAP | liquid chromatography area percent |
| LiBr | lithium bromide |
| M | molar |
| mg | milligram(s) |
| min | minutes |
| mL | milliliter(s) |
| mmol | Millimole(s) |
| MTBE | methyl tert-butyl ether |
| N | normal |
| NBP | N-butyl-2-pyrrolidone |
| ND | not determined |
| NMM | N-methylmorpholine |
| Oxyma | cyano-hydroxy-imino-acetic acid ethyl ester |
| PTFE | polytetrafluoroethylene |
| PyBOP | benzotriazole-1-yloxytripyrrolidinophoshonium hexafluorophosphate |
| PyOxim | [ethyl cyano(hydroxyamino)acetato-O²]tri-1-pyrrolidinylphosphonium |
| | hexafluorophosphate |
| Psi(Me,Me)pro | pseudoproline |
| rcf | relative centrifugal force |
| rpm | revolutions per minute |
| rt, RT | room temperature |
| TBTU | (benzotriazolyl)tetramethyluronium tetrafluoroborate |
| TEA | triethylamine |
| TFA | trifluoroacetic acid |
| THF | tetrahydrofuran |
| TIPS | triisopropylsilyl |
| Trt | trityl |
| uL, μL | microliter(s) |

### Example 1: SPPS Preparation of a Protected Variant of Peptide B from Protected Variants of Fragments 3 and 4

A protected variant of Fragment 4 (SEQ ID NO: 4) on solid support having an Fmoc on the N-terminus (Fmoc-Ser(tBu)-Gly-Gly-Gly-Gly-Ser(tBu)-Gly-Gly-Gly-Gly-Ser(tBu)-Lys(ivDde)-Solid Support (SEQ ID NO: 117); Xanthenyl linker resin) was deprotected (20% 4-methylpiperidine in DMF) to provide a free N-terminal amine. A protected variant of Fragment 3 (SEQ ID NO: 3) (Fmoc-Trp(Boc)-Leu-Val-Lys(Boc)-Gly-Gly-Gly-Gly-Gly-Gly-Gly-OH (SEQ ID NO: 56) (0.0214 mmol, 1.5 eq.) was coupled with the Fmoc-deprotected protected variant of Fragment 4 on solid support (Xanthenyl linker resin) using DIC (0.0428 mmol, 3 eq.)/Oxyma (0.0428 mmol, 3 eq.) in DMF at 50° C. overnight to yield a protected variant of Peptide E (SEQ ID NO: 8) (Fmoc-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-Solid Support (SEQ ID NO: 165) on solid support in 100% yield.

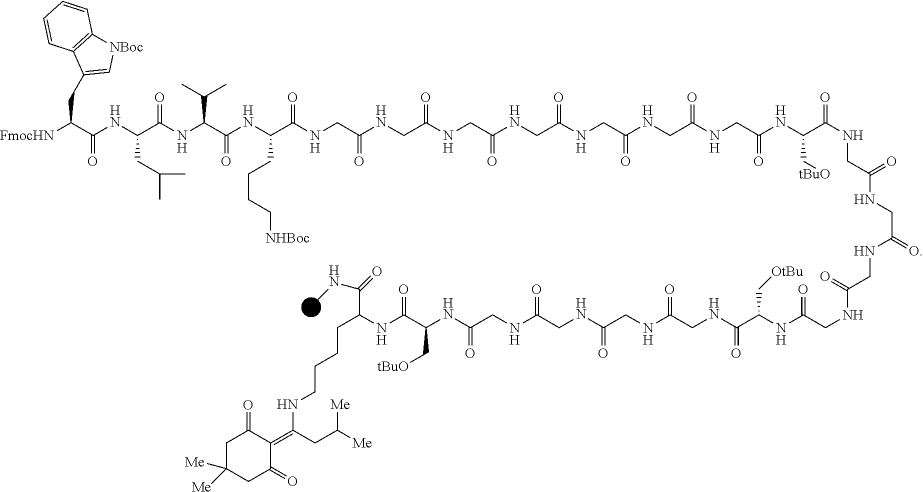

The N-terminal Fmoc of the protected variant of Peptide E on solid support was deprotected (2% DBU, 5% piperazine in DMF) to provide a free amine. Then, an Fmoc-protected alanine (Fmoc-Ala-OH) (0.378 mmol, 3 eq.) was coupled at the N-terminus of the Fmoc-deprotected variant of Peptide E on solid support using DIC (0.756 mmol, 6 eq.)/Oxyma (0.756 mmol, 6 eq.) at room temperature to yield a protected variant of Peptide B (SEQ ID NO: 9) on solid support (Fmoc-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-Solid Support (SEQ ID NO: 152) in 100% yield.

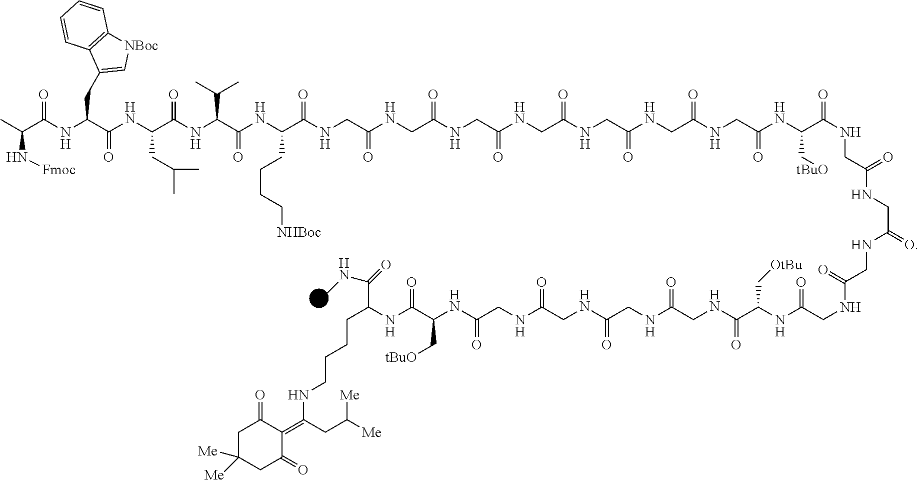

### Example 2: SPPS Preparation of a Protected Variant of Peptide B from Protected Variants of Fragments 5 and 6

A protected variant of Fragment 6 (SEQ ID NO: 6) having an Fmoc on the N-terminus on solid support (Fmoc-Gly-Gly-Gly-Gly-Ser(tBu)-Gly-Gly-Gly-Gly-Ser(tBu)-Gly-Gly-Gly-Gly-Ser(tBu)-Lys(ivDde)-Solid Support (SEQ ID NO: 138) was deprotected (20% 4-methylpiperidine in DMF) to provide a free N-terminal amine. A protected variant of Fragment 5 (SEQ ID NO: 5) (Fmoc-Ala-Trp(Boc)-Leu-Val-Lys(Boc)-Gly-Gly-Gly-OH (SEQ ID NO: 58) (20 mg, 2 eq.) was coupled under various conditions as shown in Table I to yield a protected variant of Peptide B (SEQ ID NO: 9) on solid support (Fmoc-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-Solid Support (SEQ ID NO: 152).

TABLE I

| Coupling Reagents | Solvent | Temperature | Conversion |
|---|---|---|---|
| DIC (2 eq.)/Oxyma | DMSO | rt | 97% |
| (2 eq.) | | | |
| TBTU (2 eq.)/Collidine | DMSO | rt | 95% |
| (2 eq.) | | | |
| PyOxim (2 eq.)/Collidine | DMSO | rt | 94% |
| (2 eq.) | | | |
| PyBOP (2 eq.)/Collidine | DMSO | rt | 88% |
| (2 eq.) | | | |

### Example 3: SPPS Preparation of a Protected Variant of Peptide B from Protected Variants of Fragments 3A and 4

A protected variant of Fragment 4 on solid support (Xanthenyl linker resin) having an Fmoc on the N-terminus (Fmoc-Ser(tBu)-Gly-Gly-Gly-Gly-Ser(tBu)-Gly-Gly-Gly-Gly-Ser(tBu)-Lys(ivDde)-Solid Support (SEQ ID NO: 117) was deprotected (20% 4-methylpiperidine in DMF) to provide a free N-terminal amine. Multiple deprotection cycles were required to fully remove the N-terminal Fmoc group. A protected variant of Fragment 3A (Fmoc-Ala-Trp(Boc)-Leu-Val-Lys(Boc)-Gly-Gly-Gly-Gly-Gly-Gly-Gly-OH (SEQ ID NO: 57) (1.87 g, 2 eq.) was coupled with the Fmoc-deprotected variant of Fragment 4 on solid support using TBTU (417 mg)/collidine (0.172 mL) in DMSO at room temperature to yield a protected variant of Peptide B (SEQ ID NO: 9) on solid support (Fmoc-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-Solid Support (SEQ ID NO: 152)) in a 93% yield.

### Example 4: SPPS Preparation of a Protected Variant of Peptide C (SEQ ID NO: 10)

A protected variant of Peptide B on solid support (Fmoc-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-Solid Support (SEQ ID NO: 152)) was deprotected (2% DBU, 5% piperazine in DMF) to provide a free amine at the N-terminus and was then coupled with a protected variant of Fragment 2A (Fmoc-Leu-Glu(OtBu)-Glu(OtBu)-Gln(Trt)-Ala-Ala-Lys(Boc)-Glu(OtBu)-Phe-Ile-OH (SEQ ID NO: 55) (14 mg, 2 eq.) under the conditions shown in Table II (0.014 mmol, 3 eq. for each coupling reagent/base) to yield a protected variant of Peptide C (SEQ ID NO: 10) on solid support (Fmoc-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-Solid Support (SEQ ID NO: 156).

TABLE II

| Coupling Reagents | Solvent | Temperature | Conversion |
|---|---|---|---|
| DIC/Oxyma | DMF | 50° C. | 90% |
| PyOxim/Collidine | DMF | 50° C. | 40% |
| PyBOP/Collidine | DMF | 50° C. | 67% |

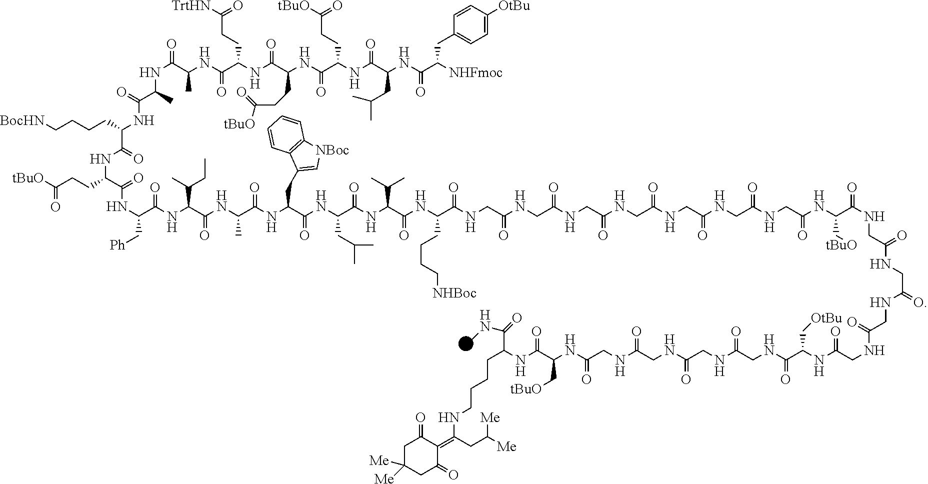

### Example 5: SPPS Preparation of Peptide A (SEQ ID NO: 7)

A protected variant of Peptide C on solid support having an Fmoc on the N-terminus was deprotected (20% 4-methylpiperidine in DMF) to provide a free N-terminal amine. A protected variant of Fragment 1A (Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH (SEQ ID NO: 100)) (0.0046 mmol, 2 eq.) was then coupled to the Fmoc-deprotected variant of Peptide C using DIC (0.0092 mmol, 4 eq.)/Oxyma (0.0092 mmol, 4 eq.) in DMF at room temperature to yield a protected variant of Peptide A on solid support in 90% yield.

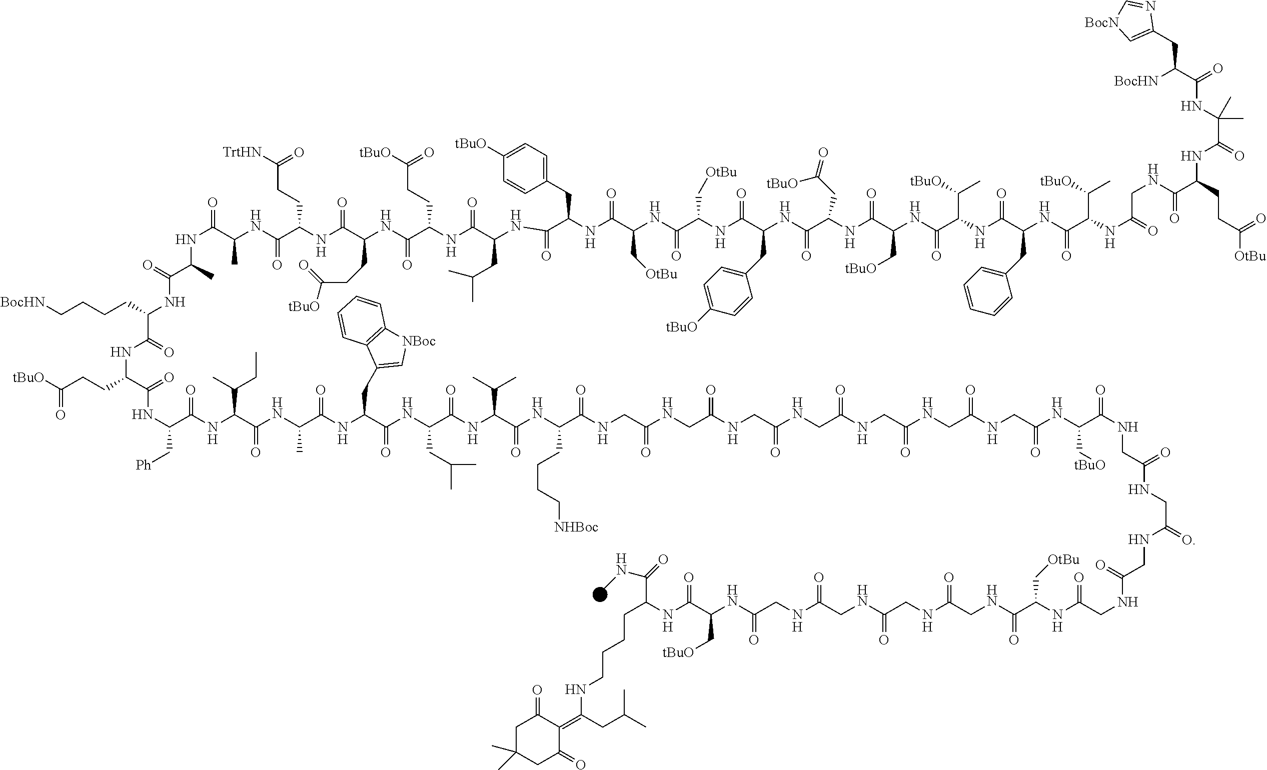

The protected variant of Peptide A was then cleaved from solid support using 95% trifluoracetic acid (TFA), 2.5% triisopropylsilane, and 2.5% water using 10-20 V with respect to the amount of resin to provide free Peptide A. Peptide A may optionally be functionalized by bromoacetylation according to the reaction conditions described in Example 11 below.

### Example 6: LPPS Preparation of a Protected Variant of Peptide B by Coupling an Fmoc-Protected Variant of Fragment 5 to a Protected Variant of Fragment 6

A protected variant of Fragment 6 (SEQ ID NO: 6) with a free amino group at the N-terminus and an amidated C-terminus (H-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 121)) and a protected variant of Fragment 5 (SEQ ID NO: 5) with a free carboxylic acid group at the C-terminus and an Fmoc-protected N-terminus (Fmoc-Ala-Trp(Boc)-Leu-Val-Lys(Boc)-Gly-Gly-Gly-OH (SEQ ID NO: 58) were coupled to form a protected variant of Peptide B with an amidated C-terminus in accordance with the reaction scheme shown in FIG. 2A (side chain protecting groups not shown). All volumes (L/kg) were calculated with respect to the weight of protected Fragment 6. The reaction vessel was charged with DMSO (2 L, 60 V), and the temperature was gradually raised to 40° C. The reaction vessel was then charged with the protected variant of Fragment 6 (39 g, 1 eq.), and the mixture was stirred until dissolved. The reaction vessel was then charged the protected variant of Fragment 5 (35 g, 1.1 eq.), and the mixture was stirred until dissolved. The coupling reagents 2,4,6-trimethylpyridine (79.86 mmol, 3 eq.) and O-(benzotriazol-1-yl)-N,N,N′,N′-tetramethyluronium tetrafluoroborate (TBTU) (79.86 mmol, 3 eq.) were charged to the reaction vessel. The reaction mixture was stirred at a rate of 200-400 rpm and a temperature of 40° C. for 18-24 h until completion of the coupling reaction. The reaction mixture was sampled for analysis at 3 h, 6 h, and 18 h. The protected variant of Fragment 6 was completely consumed after 3 h.

To cap excess protected Fragment 6, the reaction vessel was charged with acetic anhydride (1.5 eq.), and the capping reaction was allowed to proceed for 2 h. The reaction mixture was sampled for analysis at 1 h. To remove the N-terminal Fmoc protecting group from Peptide B, the reaction vessel was then charged with 4-methylpiperidine (6 V), and the reaction was allowed to proceed for 12-24 h. The reaction mixture was sampled for analysis at 3 h, 6 h, and 18 h. The reaction mixture was then cooled to 25° C., and the reaction vessel was charged with acetonitrile. The reaction mixture was stirred for 1 h and then transferred to a 5-6 L vessel and subjected to filtration under vacuum. The resulting mother liquor was collected and sampled for analysis. The resulting solids were washed with acetonitrile (60 V) and DMSO (60 V) and manually agitated to ensure thorough mixing. The filtrate was drained and sampled for analysis.

The resulting solids were subsequently washed twice with acetonitrile (60 V), with manual agitation during each wash. The filtrate was then drained and sampled for analysis. The resulting solids were dried under vacuum and a nitrogen atmosphere for a minimum of 48 h. The dried solids (crude protected Peptide B (SEQ ID NO: 9) were weighed (51.1 g, 78% adjusted yield, 98 LCAP purity), and a sample was collected for purity analysis.

### Example 7: LPPS Preparation of a Protected Variant of Peptide B by Coupling a Trt-Protected Variant of Fragment 5 to a Protected Variant Fragment 6

### Procedure 7A

A protected variant of Fragment 6 (SEQ ID NO: 6) with a free amino group at the N-terminus and an amidated C-terminus (H-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 121)) and a protected variant of Fragment 5 (SEQ ID NO: 5) with a free carboxylic acid group at the C-terminus and a Trt-protected N-terminus (Trt-Ala-Trp(Boc)-Leu-Val-Lys(Boc)-Gly-Gly-Gly-OH (SEQ ID NO: 59) were coupled to form a protected variant of Peptide B with an amidated C-terminus in accordance with the reaction scheme shown in FIG. 2B (side chain protecting groups not shown). The reaction vessel was charged with the protected variant of Fragment 6 and then charged with the protected variant of Fragment 5 (potency adjusted for Fragments 5 and 6). The reaction vessel was charged with acetonitrile and DMSO, and the reaction mixture was stirred until a homogeneous slurry formed. The slurry temperature was increased to 40° C. over a time period of at least 30 min; the slurry was then stirred for at least 1 h at 40° C. The reaction vessel was charged with 2,4,6-trimethylpyridine, followed by TBTU (3 eq.) dissolved in DMSO. The reaction mixture was stirred at 40° C. for 6-24 h until completion of the coupling reaction.

To remove the N-terminal Trt protecting group, the reaction vessel was charged with triisopropylsilane (TIPS) and TFA, and the reaction mixture was stirred at 40° C. for 6-24 h until the deprotection reaction was complete. The reaction mixture was cooled to 20° C. and filtered through a 10 μm polytetrafluoroethylene (PTFE) filter. The resulting solids were resuspended in a 4:1 (v/v) mixture of acetonitrile and dimethyl sulfoxide, with manual agitation to form a slurry, and subsequently filtered. The slurry wash procedure was repeated twice more, followed by three washes with methyl tert-butyl ether (MTBE). The resulting solids were dried under vacuum and a nitrogen atmosphere to constant weight.

### Procedure 7B

A protected variant of Fragment 6 (SEQ ID NO: 6) with a free amino group at the N-terminus and an amidated C-terminus (H-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130)) and a protected variant of Fragment 5 (SEQ ID NO: 5) with a free carboxylic acid group at the C-terminus and a Trt-protected N-terminus (Trt-Ala-Trp(Boc)-Leu-Val-Lys(Boc)-Gly-Gly-Gly-OH (SEQ ID NO: 59) were coupled to form a protected variant of Peptide B with an amidated C-terminus (H-Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me, Me)Pro)-Lys(ivDde)-NH₂ (SEQ ID NO: 147) in accordance with the reaction scheme shown in FIG. 2D. All volumes (L/kg) were calculated with respect to the weight of protected Fragment 5. The reaction vessel was charged with protected Fragments 5 and 6 as solids, followed by addition of DMSO (15 V). The reaction mixture was heated to 40° C. and stirred for 6-24 h until dissolved. The reaction vessel was then charged with PyBOP (2.5 eq.) and TEA (4 eq.). The reaction was allowed to proceed for 6 h. To remove the N-terminal Trt protecting group, the reaction vessel was charged with 1M HCl, and the reaction was allowed to proceed for 24 h at 40° C. The reaction mixture was cooled to 25° C. and then slowly added to acetonitrile (75 V) over a time period of 30 minutes. The resulting mixture was stirred for 1 h and then subjected to filtration under vacuum. The resulting solids were washed 3 times with acetonitrile (10 V) and dried under vacuum and a nitrogen atmosphere at 25° C.

### Example 8: LPPS Preparation of a N-Terminal Boc-Protected Variant of Peptide D from Protected Variants of Fragments 2B and 1

A general scheme for the preparation of a protected variant of Peptide D (SEQ ID NO: 15) with a Boc-protected N-terminus from a protected variant of Fragment 2B (SEQ ID NO: 16) and a N-terminal Boc-protected variant of Fragment 1 (SEQ ID NO: 1) is shown in FIG. 3A.

A specific example of a reaction scheme for coupling a protected variant of Fragment 2B (SEQ ID NO: 16) and a N-terminal Boc-protected variant of Fragment 1 (SEQ ID NO: 1) to obtain a protected variant of Peptide D (SEQ ID NO: 15) with a Boc-protected N-terminus is provided in FIG. 3C. A Trt group is used to mask the side chain of the N-terminal histidine residue of the protected variant of Fragment 1 that was used in the coupling reaction depicted in FIG. 3C. Specifically, a first reaction vessel was charged with the protected variant of Fragment 1 (H-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 30), 1 eq.) and THF (15 L/kg), and the reaction mixture was agitated at 20° C. for 10-20 min or until all solids were dissolved. The reaction vessel was then slowly charged with PyBOP (1.05 eq), and the vessel previously containing PyBOP and its charging port were rinsed with THF (5 L/kg). The rinse solution was added to the reaction vessel, which was then charged with 2,4,6-collidine (3 eq.) in one portion. The resulting mixture was agitated at 20° C. for 60 min to activate the protected variant of Fragment 1 and sampled for analysis by HPLC (at least 80% LCAP activated Fragment 1). A second reaction vessel was charged with a protected variant of Fragment 2B (Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 166), 1 eq.), followed by charging with DMSO (4.75 kg/L) and THF (14.25 L/kg). The resulting mixture was agitated at 20° C. for 60 min, and dissolution was confirmed by visual inspection. The Fragment 2B-containing solution was then charged into the first reaction vessel containing activated Fragment 1 at a constant rate of addition at 20° C. over a time period of 60 min. The second reaction vessel used to dissolve Fragment 2B and its addition port were rinsed with a 3:1 mixture of THF:DMSO (1 L/kg), and the rinse mixture was then transferred to the first reaction vessel. The combined reaction mixture in the first reaction vessel was agitated at 20° C. for 60 min and sampled by analysis by HPLC (less than 1% LCAP activated Fragment 2B). The reaction mixture was then charged to a third reaction vessel containing water at 20° C. over the course of 60 min with agitation. Following completion of the charge, the mixture was agitated for 120 min to precipitate the protected variant of Peptide D (Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 169). The mixture was then subjected to filtration over a 5-10 micron nylon filter. A THF (8 L/kg):water (2 L/kg) mixture was prepared in the third reaction vessel and transferred to the filter to slurry wash the residue, which was further slurry washed with THF (7 L/kg) and water (3 L/kg). The slurry wash procedure was repeated 3 times in total, and the resulting solids were dried under vacuum and a nitrogen atmosphere to constant weight.

An alternative protected variant of Peptide D (Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 157) that may be prepared by an analogous method to those shown in FIGS. 3A and 3D is provided in FIG. 3B. In this protected variant of Peptide D, Boc protecting groups are used to mask both the N-terminal amino group of Peptide D and the side chain of its N-terminal histidine residue.

### Example 9: LPPS Preparation of a Protected Variant of Peptide a Procedure 9A

A protected variant of Peptide B with an amidated C-terminus and a C-terminal lysine residue with an ivDde side chain protecting group (H-Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 147) and a protected variant of Peptide D with a Boc-protected N-terminus (Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 169)) were coupled to form a protected variant of Peptide A with a Boc-protected N-terminus, an amidated C-terminus, and a C-terminal lysine residue with an ivDde side chain protecting group in accordance with the reaction scheme shown in FIGS. 4A and 4B. The reaction vessel was first charged with a 4:1 THF:water solution (15 V). The protected variants of Peptide B (1.1 eq.) and Peptide D (1 eq.) were then charged to the reaction vessel with stirring (200 rpm) at 25° C. The reaction vessel was charged with a 4:1 THF:water solution (31 V), followed by charging of 2,4,6-collidine (6 eq.) as a neat liquid. The reaction mixture was stirred at a rate of 200 rpm and a temperature of 25° C. for 1 h. A slurry comprising HOBt (3 eq.) in a 4:1 THF:H₂O solution (1 V) was added to the reaction vessel. The HOBt slurry vessel was rinsed with a 4:1 THF:H₂O solution (1 V), and the rinse mixture was also added to the reaction vessel. The reaction vessel was then charged with a slurry comprising EDC.HCl (3 eq.) in a 4:1 THF:H₂O solution (1 V). The EDC.HCl slurry vessel was rinsed with a 4:1 THF:H₂O solution (1 V), and the rinse mixture was added to the reaction vessel. The coupling reaction was allowed to proceed overnight at 25° C. with stirring (200 rpm). The reaction mixture was subsequently added to a separate reaction vessel containing water (50 V) at 10° C. with stirring (100 rpm) at an addition rate of 1.67 V/min over a time period of 30 min. The temperature of the reaction mixture was increased to 25° C. over the course of 1 h, and the reaction mixture was then held overnight at 25° C. to precipitate the protected variant of Peptide A. The resulting mixture was transferred to a medium frit Chemglass filter and gravity filtered for 5-10 min before applying a low vacuum. The resulting solids were washed with a 3:2 water:THF solution (10 V) and dried under vacuum and a nitrogen atmosphere overnight.

### Procedure 9B

A protected variant of Peptide B and a protected variant of Peptide D were coupled to form a protected variant of Peptide A. The protected variant of Peptide B was slurry milled in acetonitrile and THF. The slurry was heated to 50° C., and the reaction vessel was subsequently charged with Cl-HOBt, the protected variant of Peptide D (Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 157), 2,4,6-collidine, and HCTU. The reaction mixture was stirred at 50° C. for 16-18 h and subsequently added to a separate reaction vessel containing water at 0° C. The resulting mixture was held overnight at 0° C. to precipitate the protected variant of Peptide A and then subjected to filtration over a PTFE filter. The resulting solids were washed with a 2:1:1 H₂O:ACN:THF solution (1×), a 4:1 THF:DMSO solution (1×), and THF (3×) and dried under vacuum and a nitrogen atmosphere to constant weight.

### Procedure 9C

The reaction vessel was slurry charged with a protected variant of Peptide B (H-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 140), 4.7 g, 1.5 eq.) that was pin-milled to reduce particle size and subsequently charged with acetonitrile, THF, and a protected variant of Peptide D (Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 169), 5 g, 1 eq.). The reaction vessel was then slurry charged with a 1:1 ACN:THF solution (14 V), and the reaction mixture was heated to 50° C. over 1 h. Cl-HOBt (1.5 eq.), 2,4,6-collidine (3 eq.), and HCTU (1.5 eq.) were added to the reaction vessel, and the resulting reaction mixture was held at 50° C. for 16-18 h. The reaction mixture was cooled to 25° C. over 1 h and subsequently transferred to a separate reaction vessel containing water at 5° C. and held at 5° C. for 2 h to precipitate the protected variant of Peptide A. The resulting reaction mixture was subjected to filtration over a nylon filter. The residue was washed twice with ACN (20 V) and dried under vacuum and a nitrogen atmosphere to constant weight (84% conversion, 82:18 dr).

### Example 10: IvDde-Deprotection of a Protected Variant of Peptide a with an ivDde Protected C-Terminal Lysine Residue

### Procedure 10A

The ivDde protecting group masking the side chain of the C-terminal lysine residue of a protected variant of Peptide A can be removed as shown in FIGS. 5A and 5B. The reaction vessel was charged with the protected variant of Peptide A (Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 174), 0.11 mmol, 1 eq.) and 1,3-dimethyl-2-imidazolidinone (30 L/kg). The reaction mixture was heated to 40° C. with agitation. Anhydrous hydrazine (2.75 mmol, 25 eq.) was then added dropwise to the reaction vessel, and the reaction mixture was agitated for 5-8 h at 40° C., with deprotection confirmed by LC/MS. The reaction vessel was subsequently charged with water (10 L/kg) over the course of 1 h and then cooled to 25° C. over 30 min. The resulting slurry was subsequently filtered under vacuum, and the resulting solids were washed with a 3:1 DMI:water solution (20 L/kg, 1×) and diethyl ether. The filter cake was then dried under vacuum and nitrogen atmosphere to constant weight. EOR analysis indicated that the ivDde deprotection reaction went to completion.

### Procedure 10B

Alternatively, the ivDde protecting group masking the side chain of the C-terminal lysine residue of a protected variant of Peptide A can be removed as shown in FIG. 5D. The reaction vessel was charged with 5% DMSO in THF (3 V) and the protected variant of Peptide A (Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 171), 1 eq.), and the mixture was stirred at 25° C. for 20 min. 50 wt % hydroxylamine in water (20 eq.) was subsequently added to the reaction vessel, and the resulting mixture was stirred for at least 20 h then sampled for analysis. The reaction was allowed to proceed until at least 98% conversion was observed by IPC. A second reaction vessel was charged with 30% heptane in MTBE (60 V) and cooled to 5° C. The reaction mixture containing ivDde deprotected Peptide A was added to the second reaction vessel over 1 h to induce precipitation of Peptide A, and the resulting solution was stirred for 1 h. The mixture was then subjected to filtration, and the resulting solids were washed with MTBE (8 V, 1×) and heptane (5V, 2×). and dried under vacuum to constant weight.

### Procedure 10C

A protected variant of Peptide A (Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 174)) (2 g, 0.324 mmol, 1 eq.) and DMI (30 V) were added to a reaction vessel. The mixture was heated to 40° C. over 10 min, aged for about 30 min, and then anhydrous hydrazine was added (8.1 mmol, 25 eq.). After stirring at 40° C. for about 24 h, water (20 mL) was added over 1 h, and the reaction mixture was cooled to 25° C. over 30 min. The resulting slurry was filtered and the filtrate was collected. The solids were washed with DMI:water (30 mL: 10 mL, 15 V: 5 V) then diethyl ether (3×10 V) and allowed to dry. The solids were re-slurried in about 30 ml of diethyl ether, filtered, and dried. No product loss was observed in the mother liquor or wash steps. An ivDde-deprotected variant of Peptide A (Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys-NH₂ (SEQ ID NO: 190) as a crystalline solid was obtained, with deprotection confirmed by LC/MS.

### Example 11: Bromoacetylation and Global Deprotection of a Protected Variant of Peptide A with a Free ε-Amino Group

### Procedure 11A

The &-amino group of the C-terminal lysine residue of a protected variant of Peptide A can be bromoacetylated as shown in FIG. 6A. A reaction vessel containing a solution of 200 eq LiBr in THF (20 V) was charged with a protected variant of Peptide A in which the &-amino group of the C-terminal lysine residue is free. N-methylmorpholine (14 eq.) and bromoacetic anhydride (7 eq.) were added with stirring to obtain a bromoacetylated protected variant of Peptide A as shown in FIG. 6B.

To globally deprotect the bromoacetylated peptide, a reaction vessel was charged with TFA (19 V), triisopropylsilane (0.5 V), and water (0.5 V) at 20° C. To the mixture was added the bromoacetylated peptide, and the mixture stirred for 90 min. The mixture was then cooled to −10° C. and MTBE (100 V) pre-chilled to −10° C. was added while maintaining the temperature below 0° C. The resulting precipitate was isolated by filtration, washed, and dried to afford the globally deprotected peptide.

### Procedure 11B

Alternatively, the s-amino group of the C-terminal lysine residue of a protected variant of Peptide A can be bromoacetylated as shown in FIG. 5D. A first reaction vessel was charged with 18% DMSO in THF (11 V), and the temperature was adjusted to 20° C. The protected variant of Peptide A (Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me, Me)Pro)-Lys-NH₂ (SEQ ID NO: 173) was then added to the reactor, and the mixture was stirred for 20 min, 2,6-lutidine (5 eq.), bromoacetic acid (10 eq.), and DIC (10 eq.) were added to the mixture, which was then stirred for at least 4 h. The bromoacetylation reaction was allowed to proceed until at least 98% conversion was observed by IPC.

A second reaction vessel was charged with 30% heptane in MTBE, which was then cooled to 5-10° C. The reaction mixture from the first reaction vessel was added to the second reaction vessel over the course of 30 min to form a precipitate. Following 30 min of agitation, the mixture was filtered, and the resulting solids were dried under vacuum to obtain a bromoacetylated protected variant of Peptide A.

The bromoacetylated protected variant of Peptide A was subsequently globally deprotected as shown in FIG. 5D. A reaction vessel was charged with TFA (14.25 V), followed by the addition of water (0.375 V) and triisopropylsilane (0.375 V). The solution was cooled to 0° C., and then the bromoacetylated protected variant of Peptide A was slowly added to the solution as a solid. The resulting reaction mixture was stirred for at least 20 h. The temperature was then reduced to −10° C., followed by the addition of MTBE over the course of 2 h to form a precipitate. The resulting mixture was subjected to filtration, and the resulting solids were washed with MTBE (15 V) and dried under vacuum to isolate the crude peptide depicted in FIG. 5E.

### Procedure 11C

Alternatively, the s-amino group of the C-terminal lysine residue of a protected variant of Peptide A can be bromoacetylated as follows. A reaction vessel was charged with a protected variant of Peptide A with a free-amino group on the side chain of its C-terminal Lys residue (Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys-NH₂ (SEQ ID NO: 190)) (6 mg, 0.001 mmol) and 2-MeTHF (20 V, 20 mL/g, 1.4 mmol). The reaction was sonicated for 5 min and LiBr was added (0.21 mmol, 210 eq.) in three portions. 2-MeTHF and LiBr were sufficient to dissolve the peptide. Bromoacetic acid (0.005 mmol, 5 eq.), Oxyma (0.0055 mmol, 5.5 eq.), and DIC (0.0055 mmol, 5.5 eq.) were added, and the reactions were incubated at room temperature with agitation. 35 UL aliquots were taken at 1.5, 4.5, and 20.5 h. 100 UL of water was added to each aliquot, and the resulting precipitate was isolated by centrifugation. A freshly prepared TFA, triisopropylsilane, and water solution (100 L, 95:2.5:2.5) was added to each pellet and incubated for 90 min. Then, 900 UL of CPME at −78° C. was added, and the resulting precipitate was pelleted and analyzed by HPLC. These conditions led to 11.7% conversion at 20.5 h.

### Procedure 11D

Alternatively, the &-amino group of the C-terminal lysine residue of a protected variant of Peptide A can be bromoacetylated as follows. A reaction vessel was charged with a protected variant of Peptide A with a free &-amino group on the side chain of its C-terminal Lys residue (Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys-NH₂ (SEQ ID NO: 190) (6 mg, 0.001 mmol) and 2-MeTHF (20 V, 20 mL/g, 1.4 mmoL). The reaction was sonicated for 5 min and LiBr was added (0.21 mmol, 210 eq.) in three portions. 2-MeTHF and LiBr were sufficient to dissolve the peptide. Bromoacetic anhydride (0.005 mmol, 5 eq.) and TEA (0.01 mmol, 10 eq.) were added, and 35 UL aliquots were taken at 1.5, 4.5, and 20.5 h. 100 UL of water was added to each aliquot, and the resulting precipitate was pelleted at 13.3 rcf for 2 min. A freshly prepared TFA, triisopropylsilane, and water solution (100 μL, 95:2.5:2.5) was added to each pellet and incubated for 90 min. Then, 900 μl of CPME at −78° C. was added, and the resulting precipitate was pelleted and analyzed by HPLC. These conditions led to 79% conversion at 1.5 h; increased incubation time did not improve conversion.

### Procedure 11E

Alternatively, the s-amino group of the C-terminal lysine residue of a protected variant of Peptide A can be bromoacetylated as follows. To a solution of 2-MeTHF (20 mL/g. 33.5 mmoL) and LiBr (4.02 mmol, 200 eq.) at room temperature was added a protected variant of Peptide A with a free &-amino group on the side chain of its C-terminal Lys residue (Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys-NH₂ (SEQ ID NO: 190) (120.9 mg, 0.0201 mmol). The solution was stirred vigorously for 2 min and sonicated for 2 min. NMM (0.2813 mmol, 14 eq.) was added followed by bromoacetic anhydride (0.1407 mmol, 7 eq.), and the reaction mixture was stirred for 105 min. Water (30 mL/g, 201.3 mmol) was added, resulting in the formation of a precipitate. The reaction mixture was filtered, washed with water (1×3 mL), and dried to furnish 109 mg (88.3% yield) on the filter and 98.5 mg upon transfer to a vial. Then, 1.5 mg of this bromoacetylated variant of Peptide A was deprotected using 20 V of freshly prepared TFA, triisopropylsilane, and water (95:2.5:2.5) solution for about 1.33 h. Then 75 V of CPME at −20° C. was added, and the resulting precipitate was collected by centrifugation. HPLC analysis of the resultant pellet showed 73% conversion to the globally deprotected Peptide A variant with an LCAP of 16 at 220 nm. See Table III for a comparison of all bromoacetylation conditions.

TABLE III

| | Scale of | | | | |
|---|---|---|---|---|---|
| | ivDde | | | Acetylation | |
| | Pep. A | Solvent, | Reagents | Conversion | LCAP |
| Entry | (mmol) | LiBr (eq.) | (eq.) | at X h | at X h |
| 1 | 0.001 | 2-MeTHF, | BrCH₂CO₂H (5.0) | 11.7% at | 2.4% |
| | | (210) | DIC/OxymaPure (5.5) | 20.5 h | |
| 2* | 0.001 | 2-MeTHF, | (BrCH₂CO)₂O (5.0) | 79% at | 16.3% |
| | | (210) | TEA (10.0) | 1.5 h | |
| 3 | 0.0201 | 2-MeTHF, | (BrCH₂CO)₂O (7.0) | 73% at | 16% (88.3% |
| | | (200) | NMM (14.0) | 1.33 h | yield pre- |
| | | | | | deprotection) |

### Example 12: Alternative Convergent LPPS Preparations of a Protected Variant of Peptide A

Protected variants of Peptide A were also prepared by liquid phase convergent peptide synthesis processes with similar reaction conditions as those described in Examples 6-9 using two alternative sets of peptide fragments (Fragments 1~4 (SEQ ID NOs: 1-4, respectively) and Fragments 1A, 2A, 3A, and 4 (SEQ ID NOs: 11, 12, 13, and 4, respectively)).

For the first alternative set of peptide fragments, a protected variant of Fragment 4 (H-Ser(tBu)-Gly4-Ser(tBu)-Gly4-Ser(tBu)-Lys(ivDde)-NH₂ (SEQ ID NO: 109)) and a protected variant of Fragment 3 (Fmoc-Trp(Boc)-Leu-Val-Lys(Boc)-Gly7-OH (SEQ ID NO: 56) were first coupled to obtain a protected variant of Peptide E (SEQ ID NO: 8). The following coupling reagents were evaluated for the coupling of protected Fragment 4 and protected Fragment 3:DIC/Oxyma, HCTU/DIEA and PyOxim/DIEA, with full conversion to protected Peptide E observed with DIC/Oxyma (Table IV).

TABLE IV

| Frag 3 | Frag 4 | | Solvent | Temperature | Time |
|---|---|---|---|---|---|
| (eq.) | (eq.) | Reagents | System | (°) | (h) |
| 1.05 | 1 | 1.4 eq PyOxim | DMSO:ACN | 50 | 20 |
| | | 4.0 eq DIEA | (4:1), 125 V | | |
| 1.35 | 1 | 2.0 eq HCTU | DMSO:ACN | 50 | 20 |
| | | 2.1 eq DIEA | (4:1), 125 V | | |
| 1.35 | 1 | 3.0 eq Oxyma | DMSO, 120 V | 50 | 20 |
| | | 3.0 eq DIC | | | |
| 1 | 1 | 3.0 eq Oxyma | DMSO, 120 V | 50 | 20 |
| | | 3.0 eq DIC | | | |

Subsequently, a protected variant of Fragment 2 (Fmoc-Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 52) was coupled with an Fmoc-deprotected variant of Peptide E (H-Trp(Boc)-Leu-Val-Lys(Boc)-Gly7-Ser(tBu)-Gly4-Ser(tBu)-Gly4-Ser(tBu)-Lys(ivDde)-NH₂ (SEQ ID NO: 160) in DMSO at 50° C. to obtain a protected variant of Peptide F (Fmoc-Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly7-Ser(tBu)-Gly4-Ser(tBu)-Gly4-Ser(tBu)-Lys(ivDde)-NH₂ (SEQ ID NO: 87). DIC/Oxyma and TBTU/2,4,6-trimethylpyridine were evaluated as coupling reagents for coupling Peptide E (1.35 eq.) and protected Fragment 2 (1 eq.), with 84% conversion (5:1 diastereomeric ratio (dr)) achieved with DIC (3 eq.)/Oxyma (3 eq.) after 24 h and 77% conversion achieved with TBTU (3 eq.)/2,4,6-trimethylpiperidine (3 eq.) after 16 h. An Fmoc-deprotected variant of Peptide F (SEQ ID NO: 85) was then coupled with a protected variant of Fragment 1 (Fmoc-His (Trt)-Aib-Glu(OtBu)-Gly-Thr(tBu)-Phe-Thr(tBu)-Ser(tBu)-Asp(OMpe)-Tyr(tBu)-Ser(tBu)-Ser(Psi(Me,Me)Pro)-OH (SEQ ID NO: 92)) in DMSO at 50° C. to obtain a protected variant of Peptide A. The following combinations of coupling reagents and bases or coupling additives (0.0048 mmol, 3 eq. each) were evaluated for the coupling of the Fmoc-deprotected variant of Peptide F (7 mg, 0.0016 mmol, 1 eq.) and protected Fragment 1 (10 mg, 0.0048 mmol, 3 eq.) in 1000 μL DMSO at 50° C.: DIC/Oxyma, EDC/Oxyma, PyBOP/2,4,6-trimethylpyridine, PyOxim/2,4,6-trimethylpyridine and TBTU/2,4,6-trimethylpyridine, with 95% conversion achieved with DIC/Oxyma (3:1 dr).

TABLE V

| | Conversion | Conversion | |
|---|---|---|---|
| Conditions | (20 h) | (40 h) | D.R. |
| Oxyma/DIC | 86 | 95 | 3:1 |
| Oxyma/EDC | 85 | 94 | 3:1 |
| TBTU/2,4,6- | ND | 94 | 3:1 |
| trimethylpyridine | | | |
| PyBOP/2,4,6- | 83 | 88 | 3:1 |
| trimethylpyridine | | | |
| PyOxim/2,4,6- | 68 | 71 | 3:1 |
| trimethylpyridine | | | |

For the second alternative set of peptide fragments, an Fmoc-deprotected variant of Fragment 4 (H-Ser(tBu)-Gly4-Ser(tBu)-Gly4-Ser(tBu)-Lys(ivDde)-NH₂ (SEQ ID NO: 109)) (42 mg, 0.034 mmol, 1.35 eq.) and a protected variant of Fragment 3A (Fmoc-Ala-Trp(Boc)-Leu-Val-Lys(Boc)-Gly7-OH (SEQ ID NO: 57) (36.5 mg, 0.0254 mmol, 1 eq.) were first coupled in DMSO (3.8 mL) at 50° C. to obtain a protected variant of Peptide B (SEQ ID NO: 144). The following combinations of coupling reagents and coupling additives or bases (0.0762 mmol, 3 eq. each) were evaluated for the coupling of protected Fragment 4 and protected Fragment 3A: DIC/Oxyma, PyBOP/2,4,6-trimethylpyridine, PyOxim/2,4,6-trimethylpyridine, and TBTU/2,4,6-trimethylpyridine, with full conversion to protected Peptide B observed with DIC/Oxyma and 96% conversion observed with TBTU/2,4,6-trimethylpyridine. Subsequently, a protected variant of Fragment 2B (Fmoc-Leu-Glu(OtBu)-Glu(OtBu)-Gln(Trt)-Ala-Ala-Lys(Boc)-Glu(OtBu)-Phe-Ile-OH (SEQ ID NO: 53) (2.4 mg, 0.0013 mmol, 1 eq.) was coupled with an Fmoc-deprotected variant of Peptide B (H-Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 140) (4.2 mg, 0.0017 mmol, 1.35 eq.) in DMSO (0.5 mL) at 50° C. to obtain a protected variant of Peptide C (SEQ ID NO: 10). The following coupling reagents were evaluated for the coupling of protected Fragment 2B and protected Peptide B, using 0.0039 mmol of each coupling reagent, coupling additive, and/or base: DIC/Oxyma, EDC/Oxyma, PyBOP/2,4,6-trimethylpyridine, PyOxim/2,4,6-trimethylpyridine, TBTU/2,4,6-trimethylpyridine, TBTU/DIEA, HCTU/DIEA, and PyOxim/DIEA, with maximum conversion observed with DIC/Oxyma (25% conversion; 2:1 dr). 9% conversion was observed with TBTU/2,4,6-trimethylpyridine, and no coupling was observed under the other tested conditions. 76% conversion was observed with DIC/Oxyma when DMF was used in place of DMSO.

An Fmoc-deprotected variant of Peptide C (H-Leu-Glu(OtBu)-Glu(OtBu)-Gln(Trt)-Ala-Ala-Lys(Boc)-Glu(OtBu)-Phe-Ile-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 172), 0.0013 mmol, 1 eq. was then coupled with a protected variant of Fragment 1A (Fmoc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH (SEQ ID NO: 99), 0.0017 mmol, 1.35 eq.) using DIC (0.0051 mmol)/Oxyma (0.0051 mmol) in DMSO or DMF (1 mL) at 50° C. to obtain a protected variant of Peptide A, with 86% conversion achieved with DIC/Oxyma in DMF (9:2:1 dr).

### Example 13: Alternative Coupling Conditions of a Trt-Protected Variant of Fragment 5 to a Protected

### Variant of Fragment 6 and Alternative Deprotections to Furnish a Protected Variant of Peptide B

### Procedure 13A: PyBOP/TEA 2 Gram-Scale One-Pot Synthesis of a Protected Variant of Peptide B.

The reaction vessel was sequentially charged with a protected variant of Fragment 6 (H-Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130)) (3 g, 1.69 mmol, 1.1 eq.), a protected variant of Fragment 5 (Trt-Ala-Trp(Boc)-Leu-Val-Lys(Boc)-Gly-Gly-Gly-OH (SEQ ID NO: 59)) (2 g, 1.54 mmol, 1 eq.), DMSO (2.4 L), and ACN (0.6 L). The mixture was stirred at 40° C. until homogeneous. PyBOP (3.85 mmol, 2.5 eq.) and TEA (6.16 mmol, 4.0 eq.) were added, and the reaction was stirred for about 7 h at 40° C.

Then, HCl (4.62 mmol, 3.0 eq., 1 N in water) was added, and the reaction was stirred for 24 h at 40° C. The reaction mixture was cooled to 25° C. and added into ACN (100 mL, 50V) over 10 min. After stirring for 10 min, the product was filtered and washed with ACN (2×, 100 mL, 50V). The filtered solid was then washed with water (100 mL, 50V) and dried to afford a protected variant of Peptide B with a free N-terminal amino group (H-Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 147).

### Procedure 13B: Evaluation of HCl Stock Concentration Effects on Trt Deprotection

In three reactors, a protected variant of Fragment 6 (H-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130); 79.8% (w/w); 151 mg; 0.0847 mmol; 1.1 eq.) was charged as a solid to a 10 mL vial, followed by the addition of a protected variant of Fragment 5 (Trt-Ala-Trp(Boc)-Leu-Val-Lys(Boc)-Gly-Gly-Gly-OH (SEQ ID NO: 59); 94.7% (w/w); 100 mg; 0.077 mmol; 1 eq.), DMSO (1.2 mL; 15 L/kg), and ACN (0.3 mL; 3 L/kg). The resulting mixture was stirred (700 rpm) at 40° C. for 15 min to obtain a homogenous mixture. Then PyBOP (2.5 eq.) and TEA (4 eq.) were charged as powders, and the coupling reaction was allowed to proceed with stirring (550 rpm) at 40° C. The reaction mixture was sampled for analysis at 4 h, with 100% conversion observed by 4 h. Next, HCl (3 eq.) was added to each reactor from one of three stock solutions with variable water content (1 N HCl, 2 N HCl, or 37% HCl) to selectively remove the N-terminal trityl protecting group on Fragment 5. The mixtures were aged for 24 h and sampled for analysis. 97%, 91%, and 66% trityl deprotection rates were observed for 1 N HCl, 2 N HCl, and 37% HCl, respectively.

Conditions for coupling a protected variant of Fragment 6 (Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me, Me)Pro)-Lys(ivDde)-NH₂ (SEQ ID NO: 130) (151 mg, 0.085 mmol, 1.1 eq.) and a protected variant of Fragment 5 (Trt-Ala-Trp(Boc)-Leu-Val-Lys(Boc)-Gly-Gly-Gly-OH (SEQ ID NO: 59) (100 mg, 0.077 mmol, 1.0 eq.) were assessed, as shown in Table VI. The fragments were dissolved in solvent, then coupling reagent and base were added. PyBOP (0.193 mmol, 2.5 mmol) and TEA (0.308 mmol, 4.0 eq.) were used as coupling reagent and base, respectively, except for entry 2 which used HOPO (0.193 mmol, 2.5 mmol) and DIC (0.308 mmol, 4.0 eq.). All coupling reactions were conducted at 40° C. for 2 h. Then, HCl was added (0.231, 3.0 eq., 1 N in water), and the reaction mixture was stirred at 40° C. for 24 h. The amount of the desired destrityl Peptide B variant to Psi hydrolyzed side product (Impurity-Peptide B) was measured by HPLC.

TABLE VI

| | | | Coupling | Deprotection | Impurity- |
|---|---|---|---|---|---|
| | | | Conversion (%) | Conversion (%) | Pep. B:DesTrt- |
| Entry | Solvents | Vol. | at 24 h | at 24 h | Pep. B |
| 1 | DMSO:MeOH | 15 V | 100 | 91 | 30:70 |
| | (4:1) | | | | |
| 2* | DMSO:MeOH | 15 V | 92 | 98 | 32:68 |
| | (4:1) | | | | |
| 3 | DMSO:MeOH | 15 V | 100 | 99 | 30:70 |
| | (1:1) | | | | |
| 4 | DMSO:MeOH | 30 V | 100 | 97 | 17:83 |
| | (1:1) | | | | |
| 5 | DMSO:IPA | 30 V | 8 | ND | ND |
| | (1:1) | | | | |

### Procedure 13D: Solvent and Acid Equivalent Screen for One-Pot Synthesis of Protected Variant of Peptide B and Impact on Psi Hydrolysis.

A protected variant of Fragment 6 (H-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130)) (139 mg, 0.085 mmol, 1.1 eq.) and a protected variant of Fragment 5 (Trt-Ala-Trp(Boc)-Leu-Val-Lys(Boc)-Gly-Gly-Gly-OH (SEQ ID NO: 59) (100 mg, 0.077 mmol, 1.0 eq.) were dissolved with 3 mL of a solvent system as shown in Table VII, heated to 40° C., and stirred for 15 min. Then, PyBOP (0.193 mmol, 2.5 mmol) and TEA (0.308 mmol, 4 eq.) were added. The resulting mixture was stirred for 2 h. Then, HCl (1 M in water) was added, and the reaction mixture was stirred at 40° C. for 24 h. The amount of desired destrityl Peptide B variant to Peptide B impurity was measured by HPLC, as was a side protect of Boc removal of the Trp residue (DesBoc-Trp-Pep B).

TABLE VII

| | | Coupling | | Deprotection | Impurity- | DesBoc-Trp- |
|---|---|---|---|---|---|---|
| | | Conversion | | Conversion | Pep. B:DesTrt- | Pep. B:DesTrt- |
| Entry | Solvents | (%) at 2 h | Eq. HCl | (%) at 24 h | Pep. B | Pep. B |
| 1 | DMSO:MeOH | 100 | 3.25 | 100 | 18:82 | 2 |
| | (1:1) | | | | | |
| 2 | DMSO:MeOH | 100 | 3.5 | 100 | 21:79 | 2 |
| | (1:1) | | | | | |
| 3 | DMSO:ACN | 100 | 3.0 | 100 | 26:74 | 2 |
| | (1:1) | | | | | |
| 4 | DMSO:ACN | 100 | 2.0 | 91 | 23:77 | 2 |
| | (1:1) | | | | | |
| 5 | DMSO:ACN | 100 | 1.5 | 68 | 16:84 | 2 |
| | (1:1) | | | | | |

### Procedure 13E: Acid Equivalent Screen for One-Pot Synthesis of Protected Variant of Peptide B and Impact on Psi Hydrolysis.

All reactions were conducted as described in Example 13D, using 3 mL of DMSO:MeOH (1:1), up to the HCl addition. Each reaction was charged with HCl (1 M in water) under the conditions shown in Table VIII. The protected variant of Peptide B with a free N-terminal amino group, Psi hydrolysis impurity, and DesBoc-Trp-Peptide B products were analyzed by HPLC.

TABLE VIII

| | | | Trt | | |
|---|---|---|---|---|---|
| | | | Deprotection | Impurity- | DesBoc-Trp- |
| | Eq. | Time | Conversion | Pep. B:DesTrt- | Pep. B:DesTrt- |
| Entry | HCl | (h) | (%) at 24 h | Pep. B | Pep. B |
| 1 | 3.00 | 20 | 99 | 24:76 | 2 |
| | | 24 | 100 | 24:76 | 2 |
| 2 | 3.10 | 20 | 99 | 25:75 | 2 |
| | | 24 | 100 | 26:74 | 2 |
| 3 | 3.25 | 20 | 100 | 23:77 | 2 |
| | | 24 | 100 | 28:72 | 2 |
| 4 | 3.50 | 20 | 100 | 25:75 | 2 |
| | | 24 | 100 | 30:70 | 2 |
| 5 | 3.75 | 20 | 100 | 29:71 | 2 |
| | | 24 | 100 | 23:77 | 2 |
| 6 | 4.00 | 20 | 100 | 28:72 | 2 |
| | | 24 | 100 | 37:63 | 2 |

### Procedure 13F: Varied Gram-Scale Syntheses of Peptide B Variant

A protected variant of Fragment 6 (H-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130), 1.4 g, 0.85 mmol, 1.1 eq.), a protected variant of Fragment 5 (Trt-Ala-Trp(Boc)-Leu-Val-Lys(Boc)-Gly-Gly-Gly-OH (SEQ ID NO: 59), 1 g, 0.77 mmol, 1 eq.), DMSO (15 mL), and MeOH (15 mL) were charged to one of three reaction vessels, and the resulting mixture was heated to 30° C. or 40° C., and stirred for 15 min until homogeneous. Then, PyBOP (1 g, 1.91 mmol, 2.5 eq.) and TEA (430 UL, 3.08 mmol, 4 eq.) were added, and the reaction mixture was stirred at the temperature noted in Table IX for 3 h. The coupling conversion was assessed by HPLC, as shown in Table IX. Then, HCl (1 N in water) was added at the equivalence noted in Table IX, and the reaction mixture stirred 24 h at the noted temperature. Selective removal of the N-terminal Trt protecting group of the protected variant of Peptide B was assessed by HPLC, as shown in Table IX.

TABLE IX

| | | Coupling/ | Coupling | Deprotection |
|---|---|---|---|---|
| | | Deprotection | Conversion | Conversion |
| Entry | Eq. HCl | Temperature (° C.) | (%) at 2 h | (%) at 24 h |
| 1 | 3.25 | 40 | 100 | 100 |
| 2 | 3.0 | 40 | 100 | 100 |
| 3 | 3.25 | 30 | 100 | >99 |

To isolate the protected variant Peptide B with a free N-terminal amino group variant at scale, the impact of temperature and anti-solvent was assessed under the conditions shown in Table X. The reaction mixture was brought to the indicated temperature, then the anti-solvent was added (reverse or direct) at a rate of 1.5 mL/min. The resulting slurry was stirred at the indicated temperature for 20 min, filtered, and the solid washed twice with antisolvent (70 V). The resulting products formed were analyzed, as reported in Table XI.

TABLE X

| | | | Isolation | Filter | Wash times |
|---|---|---|---|---|---|
| | | Mode of | Temperature | time | 1 and 2 |
| Entry | Antisolvent | Addition | (° C.) | (min) | (min) |
| 1 | Ethanol | Reverse | 20 | 59 | 8, 6 |
| 2* | Ethanol | Direct | 25 | 3 | 7, 6 |
| 3 | Ethyl Acetate | Reverse | 25 | 30 | 1, 1 |

TABLE XI

| | | | | | | | | | | Yield (%) |
|---|---|---|---|---|---|---|---|---|---|---|
| | LCAP of final solids | | | | | | | | | Yield (not |
| | | Pep. B - | Pep. B - | Epi- | DesPsi- | DesPsi- | DesPsi- | DesBoc- | DesBoc- | adjusted |
| No. | FragA | NHTrt | NH₂ | Pep. B | Pep. B 1 | Pep. B 2 | Pep. B 3 | Pep. B 1 | Pep. B 2 | for potency) |
| 1 | 0.43 | 0 | 64.1 | 1 | 5.6 | 5.3 | 0.95 | 4.2 | 0.44 | 94 |
| 2 | 0.36 | 0 | 70.8 | 0.64 | 3.6 | 4.1 | 0.55 | 1.9 | 0.82 | 87 |
| 3 | 1.41 | 0.21 | 71.5 | 1.34 | 2.8 | 3.4 | 0.41 | 1.2 | 0.62 | 94 |

Direct addition of ethanol resulted in faster filtration (entry 2), and lower temperature for the addition of the anti-solvent provided the highest amount of deprotected Peptide B (entry 3).

### Example 14: Alternative Conditions for Coupling of a Protected Variant of Peptide with a Protected Variant of Peptide D

### Procedure 14A: Additive, Base, and Coupling Agent Screen

A protected variant of Peptide D (Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 157)) (1 eq.) and a protected variant of Peptide B (Ala-Trp(Boc)-Leu-Val-Lys(Boc)-Gly-Gly-Gly-Gly-Gly-Gly-Gly-Ser(tBu)-Gly-Gly-Gly-Gly-Ser(tBu)-Gly-Gly-Gly-Gly-Ser(tBu)-Lys(ivDde)-NH₂ (SEQ ID NO: 140) (1.4 eq.) were coupled under various conditions as shown in Table XII to form a protected variant of Peptide A. Solvent was added to the two peptides, followed by base solution, additive solution, and coupling reagent solution. All additive/coupling agent solutions had a concentration of 50 mg/ml except for 6-Cl-HOBt (12.5 mg/mL). The reactions were stirred for 24 h at the temperature noted. Then, a sample of each was taken (0.4 mL), added to water (1.5 mL), and pelleted by centrifugation. The crude products were globally deprotected by stirring in a TFA, triisopropylsilane, and water solution (1 mL, 95:2.5:2.5) for 3.5 h, then the resulting products were analyzed by HPLC.

TABLE XII

| Entry | Pep. B/Pep. D | Conditions | Pep. A/Pep. B | Pep. A/Pep. D | Pep. A |
|---|---|---|---|---|---|
| (° C.) | amount (mg) | (eq.) | LCAP ratio | LCAP % ratio. | dr |
| 1 | 48/50 | HCTU (1.5) | 44/54 | 91/9 | 84:16 |
| (25) | | Cl-HOBt (1.5) | | | |
| | | 2,4,6-Collidine (3) | | | |
| 2 | 48/50 | DIC (2) | 51/49 | 69/31 | 89:11 |
| (25) | | Oxyma (2) | | | |
| 3 | 48/50 | PyBOP (2) | 50/50 | 76/24 | 81:19 |
| (25 | | 2,4,6-Collidine (3) | | | |
| 4 | 48/50 | PyBOP (2) | 51/49 | 74/26 | 88:12 |
| (25) | | HOBt (3) | | | |
| | | 2,4,6-Collidine (3) | | | |
| 5 | 48/50 | PyOxim (2) | 67/33 | 53/47 | 85:15 |
| (25) | | 2,4,6-Collidine (3) | | | |
| 6 | 48/50 | PyOxim (2) | 73/27 | 41/59 | 93:7 |
| (25) | | Oxyma (2) | | | |
| | | 2,4,6-Collidine (3) | | | |
| 7 | 48/50 | HCTU (1.5) | 40/60 | 95/5 | 80:20 |
| (40) | | CI-HOBt (1.5) | | | |
| | | 2,4,6-Collidine (3) | | | |
| 8 | 48/50 | DIC (2) | 42/58 | 88/12 | 86:14 |
| (40) | | Oxyma (2) | | | |
| 9 | 48/50 | PyBOP (2) | 40/60 | 89/11 | 73:27 |
| (40) | | 2,4,6-Collidine (3) | | | |
| 10 | 48/50 | PyBOP (2) | 41/59 | 89/11 | 80:20 |
| (40) | | HOBt (3) | | | |
| | | 2,4,6-Collidine (3) | | | |
| 11 | 48/50 | PyOxim (2) | 65/35 | 59/41 | 80:20 |
| (40) | | 2,4,6-Collidine (3) | | | |
| 12 | 48/50 | PyOxim (2) | 59/41 | 63/37 | 89:11 |
| (40) | | Oxyma (2) | | | |
| | | 2,4,6-Collidine (3) | | | |

### Procedure 14B: Solvent Screen for Formation of a Protected Variant of Peptide A.

A protected variant of Peptide D (Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 169)) (83 mg, 1 eq., dr 99:1) and a protected variant of Peptide B (HCl+Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 147) (50 mg, 1 eq.) were reacted under various conditions as shown in Table XIII. Solvent (2.8 mL; 35 V with respect to Peptide D) and 2,4,6-collidine (0.3 mL, 50 mg/mL, 6 eq.) were added, and the mixtures were stirred at 25° C. for 30 min. Then, Cl—HOBt* (11 mg; 3 eq.) and HCTU* (26 mg, 3 eq.) were added, and the reactions were stirred at 25° C. for about 29 hours. After which, a sample (0.3 mL) of each reaction was added to water (1.5 mL) and pelleted by centrifugation. The pellets were globally deprotected by stirring in a TFA, triisopropylsilane, and water solution (1 mL, 95:2.5:2.5) for 4 h and analyzed by HPLC. *Entry 10 received 6 and 12 initial eq. of HCTU and Cl—HOBt, respectively, and Entry 11 received 3 and 6 addition eq. of HCTU and Cl—HOBt, respectively, after 24 hours.

TABLE XIII

| | | Conversion | |
|---|---|---|---|
| Entry | Solvent | at 29 h (%) | dr |
| 1 | THF:H₂O | 73 | 94:6 |
| | (4:1) | | |
| 2 | THF:H₂O | 16 | 94:6 |
| | (9:1) | | |
| 3 | THF:H₂O | 5 | ND |
| | (19:1) | | |
| 4 | THF | 0 | ND |
| 5 | THF:H₂O | 64 | 93:7 |
| | (4:1) | | |
| 6 | THF:H₂O | 71 | 88:12 |
| | (7:3) | | |
| 7 | THF:H₂O | 18 | 79:21 |
| | (6:4) | | |
| 8 | THF:H₂O | NR | N/A |
| | (1:1) | | |
| 9 | THF:H₂O | NR | N/A |
| | (1:3) | | |
| 10* | THF:H₂O | 50 | 92:8 |
| | (4:1) | | |
| 11** | THF:H₂O | 71 | 93:7 |
| | (4:1) | | |
| 12 | THF:DMSO | NR | N/A |
| | (4:1) | | |
| 13 | THF:DMSO | NR | N/A |
| | (9:1) | | |
| 14 | THF:DMSO | NR | N/A |
| | (19:1) | | |

### Procedure 14C: Scale Up EDC/HOBt Coupling to Provide a Protected Variant of Peptide A

To a 4:1 THF:H₂O (150 mL) solvent mixture was added a protected variant of Peptide D (Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 169)) (10 g, 1 eq.) and a protected variant of Peptide B (HCl+Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 147)) (8.4 g, 1.4 eq.). Additional 4:1 THF:H₂O (310 mL) was added, and the mixture was stirred at 25° C. for about 20 min. Then, 2,4,6-collidine (2 mL, 6 eqiv.) was added, and the mixture was stirred for about 1 h. A HOBt (1.6 g, 4.2 eq.) slurry in 4:1 THF:H₂O (10 mL) was added, and 4:1 THF:H₂O (10 mL) was used to rinse the slurry vial. Next, an EDC.HCl (2 g, 4.2 eq.) slurry in 4:1 THF:H₂O (10 mL) was added, and 4:1 THF:H₂O (10 mL) was used to rinse the slurry vial. The reaction mixture was stirred for 22 h at 25° C. The reaction mixture was reverse added over 30 min into a 10° C. H₂O solution (500 mL), stirred, then warmed to 25° C. after precipitation. The reaction mixture was filtered to collect the resulting solid. A sample of the resulting solid (10 mg) was subjected to deprotection using a TFA, triisopropylsilane, and water solution (1 mL. 95:2.5:2.5) for 3 h then analyzed by HPLC. Conversion of the protected variant of Peptide B was greater than 99%; the ratio of Peptide A to Peptide B in the isolated solid was 95:5; peptide recovery was 83%; Peptide A LCAP was 33%; LCAP adjusted yield of Peptide A was 77%; and dr of deprotected Peptide B was 82:18.

### Example 15: Screening Solvent, Coupling Reagent, and Activation Time Conditions for Coupling

### Protected Variants of Fragment 1A and Fragment 2A

To a stirred solution comprising a protected variant of Fragment 1A (Boc-His (Boc)-Aib-Glu(OtBu)-Gly-Thr(tBu)-Phe-Thr(tBu)-Ser(tBu)-Asp(OtBu)-Tyr(tBu)-Ser(tBu)-Ser(tBu)-Tyr(tBu)-OH (SEQ ID NO: 95), 100% (w/w); 0.013 mmol; 1.1 eq.) in DMF (100% (w/w) or DMSO (25 mL/g, 7 mmol, 100% (w/w)) was added TBTU (99% (w/w), 0.013 mmol, 1.1 eq.), HCTU (99% (w/w); 0.013 mmol; 1.1 eq.), HATU (98% (w/w), 0.013 mmol, 1.1 eq.), or PyBOP (100% (w/w); 0.013 mmol; 1.1 eq.). The resulting solution was stirred at 40° C. for 2 h or overnight to activate the protected variant of Fragment 1A, followed by the addition of a solution comprising a protected variant of Fragment 2A (H-Leu-Glu(OtBu)-Glu(OtBu)-Gln(Trt)-Ala-Ala-Lys(Boc)-Glu(OtBu)-Phe-Ile-OH (SEQ ID NO: 29), 100% (w/w); 0.012 mmol; 1 eq.) in DMSO (100% (w/w) or DMF (25 mL/g, 6.5 mmol, 100% (w/w). The reaction mixture comprising Fragment 1A and Fragment 2A was stirred at 40° C. for 18 h, then sampled for analysis by HPLC. 20 h activation led to little to no conversion, and significant racemization was observed for all conditions (Table XIV; conversion relative to Fragment 2A).

TABLE XIV

| | | Coupling | Activation | Conversion | |
|---|---|---|---|---|---|
| Experiment No. | Solvent | Reagent | Time | (%) | D.R. |
| 1 | DMSO | TBTU | 2 h | 76 | 1:1 |
| 2 | DMSO | TBTU | 20 h | 5 | 1:1 |
| 3 | DMSO | HCTU | 2 h | 6 | 1:1 |
| 4 | DMSO | HCTU | 20 h | 0 | — |
| 5 | DMSO | PyBOP | 2 h | 57 | 1:1 |
| 6 | DMSO | PyBOP | 20 h | 4 | 1:1 |
| 7 | DMSO | HATU | 2 h | 7 | 1:1 |
| 8 | DMSO | HATU | 20 h | 0 | — |
| 9 | DMF | TBTU | 2 h | 91 | 1:1 |
| 10 | DMF | TBTU | 20 h | 0 | — |
| 11 | DMF | HCTU | 2 h | 27 | 1:1 |
| 12 | DMF | HCTU | 20 h | 0 | — |
| 13 | DMF | PyBOP | 2 h | 77 | 29:71 |
| 14 | DMF | PyBOP | 20 h | 22 | 46:54 |
| 15 | DMF | HATU | 2 h | 29 | 1:1 |
| 16 | DMF | HATU | 20 h | 0 | — |

### Example 16: Screening Solvent and Coupling Reagent Conditions for Coupling Protected Variants of

### Fragment 1 and Fragment 2

To a stirred solution comprising a protected variant of Fragment 1 (Boc-His (Trt)-Aib-Glu(OtBu)-Gly-Thr(tBu)-Phe-Thr(tBu)-Ser(tBu)-Asp(OMpe)-Tyr(tBu)-Ser(tBu)-Ser(Psi(Me,Me)Pro)-OH (SEQ ID NO: 84), 100% (w/w); 0.011 mmol; 1.1 eq.) in DMF (100% (w/w) or DMSO (25 mL/g, 7 mmol, 100% (w/w)) was added TBTU (99% (w/w)), HCTU (99% (w/w); 0.011 mmol; 1.1 eq.), or PyBOP (100% (w/w); 0.011 mmol; 1.1 eq.). The resulting solution was stirred at 40° C. for 2 h or overnight to activate the protected variant of Fragment 1, followed by the addition of a solution comprising a protected variant of Fragment 2 (H-Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 28), 100% (w/w); 0.01 mmol; 1 eq.) in DMSO (100% (w/w) or DMF (25 mL/g, 6.5 mmol, 100% (w/w)). The reaction mixture comprising Fragment 1 and Fragment 2 was stirred at 40° C. for 18 h, then sampled for analysis by HPLC. Coupling reactions in DMSO showed minimal conversion, potentially due to poor solubility, while the coupling reaction using PyBOP in DMF showed high conversion with minimal racemization (Table XV; conversion relative to Fragment 2).

TABLE XV

| | | | Activation | Conversion | |
|---|---|---|---|---|---|
| Experiment No. | Solvent | Reagent | Time (h) | (%) | D.R. |
| 1 | DMSO | TBTU | 2 | 2 | — |
| 2 | DMSO | TBTU | 20 | 0 | — |
| 3 | DMSO | HCTU | 2 | 0 | — |
| 4 | DMSO | HCTU | 20 | 0 | — |
| 5 | DMSO | PyBOP | 2 | 0 | — |
| 6 | DMSO | PyBOP | 20 | 0 | — |
| 7 | DMF | TBTU | 2 | 100 | 1:1 |
| 8 | DMF | TBTU | 20 | 0 | — |
| 9 | DMF | HCTU | 2 | 40 | >99:1 |
| 10 | DMF | HCTU | 20 | 0 | — |
| 11 | DMF | PyBOP | 2 | 100 | 99:1 |
| 12 | DMF | PyBOP | 20 | 3% | — |

### Example 17: Screening Solvent and Temperature Conditions for Coupling Protected Variants of

Fragment 1 and Fragment 2 with PyBOP/2,4,6-Collidine

To a stirred solution comprising a protected variant of Fragment 1 (Boc-His (Trt)-Aib-Glu(OtBu)-Gly-Thr(tBu)-Phe-Thr(tBu)-Ser(tBu)-Asp(OMpe)-Tyr(tBu)-Ser(tBu)-Ser(Psi(Me,Me)Pro)-OH (SEQ ID NO: 84), 100% (w/w); 0.011 mmol; 1.1 eq.) in DMF (100% (w/w)) or DMAc (100% (w/w) was added PyBOP (100% (w/w); 0.011 mmol; 1.1 eq.). The solution was then stirred at 20° C. or 40° C. for about 2 h to activate the protected variant of Fragment 1, followed by the addition of a solution comprising a protected variant of Fragment 2 (H-Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 28), 100% (w/w); 0.01 mmol; 1 eq.) in DMF (25 mL/g, 6.5 mmol, 100% (w/w)) or DMAc (100% (w/w) (100 mass %). The resulting solution was stirred at 20° C. or 40° C. for 18 h, then sampled for analysis by HPLC. Coupling reactions in DMF resulted in complete conversion but increased racemization relative to coupling reactions in DMAc (Table XVI, conversion relative to Fragment 2). In addition, increased conversion and reduced racemization were observed at a coupling temperature of 20° C.

TABLE XVI

| | | Reaction | Conversion | |
|---|---|---|---|---|
| Experiment No. | Solvent | Temperature (°) | (%) | D.R. |
| 1 | DMF | 40 | 100 | 59:41 |
| 2 | DMF | 20 | 100 | 93:7 |
| 3 | DMAc | 40 | 72 | 99:1 |
| 4 | DMAc | 20 | 85 | >99:1 |

### Example 18: Screening Reaction Times for Protected Variants of Fragment 1 and Fragment 2 Coupled with PyBOP/2,4,6-Collidine in DMAc

To a stirred solution comprising a protected variant of Fragment 1 (Boc-His (Trt)-Aib-Glu(OtBu)-Gly-Thr(tBu)-Phe-Thr(tBu)-Ser(tBu)-Asp(OMpe)-Tyr(tBu)-Ser(tBu)-Ser(Psi(Me,Me)Pro)-OH (SEQ ID NO: 84), 100% (w/w); 0.015 mmol; 1.1 eq.) in wet DMAc was added PyBOP (100% (w/w); 0.015 mmol; 1.1 eq.). The resulting solution was stirred at 20° C. for 2 h, followed by the addition of a solution comprising a protected variant of Fragment 2 (Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 28), 100% (w/w); 0.014 mmol; 1 eq.) in DMAc (25 mL/g, 7.3 mmol, 100% (w/w). The reaction mixture comprising Fragment 1 and Fragment 2 was stirred at 20° C. for 18 h, then sampled for analysis by HPLC. The crude reaction mixture was quenched by 95% TFA, 2.5% TIPS, 2.5% H₂O for analysis. The coupling reaction went to completion within 30 min at 20° C. with minimal alanine racemization (Table XVII; conversion rates relative to Fragment 2). The use of wet DMAc may have prevented complete conversion of Fragment 2.

TABLE XVII

| Reaction Time | Conversion (%) | D.R. |
|---|---|---|
| 30 min | 88 | >99:1 |
| 1 h | 87 | >99:1 |
| 2 h | 88 | >99:1 |
| 18 h | 88 | >99:1 |

### Example 19: Screening Activation Times for Protected Variants of Fragment 1 and Fragment 2 Coupled with PyBOP/2,4,6-Collidine in DMAc

To a stirred solution comprising a protected variant of Fragment 1 (Boc-His (Trt)-Aib-Glu(OtBu)-Gly-Thr(tBu)-Phe-Thr(tBu)-Ser(tBu)-Asp(OMpe)-Tyr(tBu)-Ser(tBu)-Ser(Psi(Me,Me)Pro)-OH (SEQ ID NO: 84)), 100% (w/w); 0.011 mmol; 1.1 eq.) in wet DMAc was added PyBOP (100% (w/w); 0.011 mmol; 1.1 eq.) then 2,4,6-collidine (100% (w/w); 0.03 mmol; 3 eq.). The resulting solution was stirred at 20° C. for about 30 min, 1 h, or 2 h, followed by the addition of a solution comprising a protected variant of Fragment 2 (Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 28), 100% (w/w); 0.01 mmol; 1 eq.) in DMAc. The reaction mixture comprising Fragment 1 and Fragment 2 was stirred at 20° C. for 30 min, then sampled for analysis by HPLC. The crude reaction mixture was quenched by 95% TFA, 2.5% TIPS, 2.5% H₂O for analysis (Table XVIII, conversion rates relative to Fragment 2). The use of wet DMAc may have prevented complete conversion of Fragment 2.

TABLE XVIII

| Activation Time | Conversion (%) | D.R. |
|---|---|---|
| 30 min | 96% | >99:1 |
| 1 h | 92% | >99:1 |
| 2 h | 85% | >99:1 |

### Example 20: Coupling Protected Variants of Fragment 1 and Fragment 2 with PyBOP/2,4,6-Collidine in DMF

To a stirred solution comprising a protected variant of Fragment 1 (Boc-His (Trt)-Aib-Glu(OtBu)-Gly-Thr(tBu)-Phe-Thr(tBu)-Ser(tBu)-Asp(OMpe)-Tyr(tBu)-Ser(tBu)-Ser(Psi(Me,Me)Pro)-OH (SEQ ID NO: 84)), 100% (w/w); 0.011 mmol; 1.1 eq.) in DMF (100% (w/w)) was added PyBOP (100% (w/w); 0.011 mmol; 1.1 eq.) then 2,4,6-collidine (100% (w/w); 0.03 mmol; 3 eq.). The resulting solution was stirred at 20° C. for 30 min, 1 h, or 2 h, followed by the addition of a solution comprising a protected variant of Fragment 2 (Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 28), 100% (w/w); 0.01 mmol; 1 eq.) in DMF. The reaction mixture comprising Fragment 1 and Fragment 2 was stirred at 20° C. for about 30 min, 1 h, 2 h, or overnight. The coupling reaction went to completion within 30 min at 20° C. (Table XIX; conversion rates relative to Fragment 2). For overnight incubation, one reaction was allowed to proceed without further additives, one reaction was performed with 1.1 eq. PyBOP added after 2 h, and one reaction was performed with TFA added to reach 1% TFA in the crude reaction mixture after 2 h. After overnight incubation, the diastereomeric ratio (d.r.) for the product of the quenched reaction was 88:12, while the unquenched reaction product had a d.r. of 67:33. The d.r. for the product of the reaction performed with a second PyBOP charge was 56:44.

All analyses were done by quenching the crude reaction mixture into a mixture of 95% TFA, 2.5% TIPS, 2.5% H₂O. The resulting mixture was then analyzed by HPLC to check conversion and D.R. selectivity.

TABLE XIX

| Activation Time | Conversion (%) | D.R. |
|---|---|---|
| 30 min | 100 | 88:12 |
| 1 h | 100 | 92:8 |
| 2 h | 100 | 93:7 |

### Example 21: Screening Solvent Conditions for Coupling Protected Variants of Fragment 1 and Fragment

2B with PyBOP/2,4,6-Collidine

To a stirred solution comprising a protected variant of Fragment 1 (Boc-His (Trt)-Aib-Glu(OtBu)-Gly-Thr(tBu)-Phe-Thr(tBu)-Ser(tBu)-Asp(OMpe)-Tyr(tBu)-Ser(tBu)-Ser(Psi(Me,Me)Pro)-OH (SEQ ID NO: 84), 1.1 eq., 0.012 mmol, 100% (w/w) in 0.2 mL DMF, DMSO, THF, MeCN, or DMI was added PYBOP (0.9 eq.; 0.0094 mmol; 100% (w/w) in 10 UL DMF. The resulting solution was stirred at 20° C. for 30 min to activate the protected variant of Fragment 1. Then a solution comprising a protected variant of Fragment 2B (H-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 30), 20 mg, 1 eq., 0.01 mmol, 100% (w/w)) in the same solvent (0.2 mL) was added to the activated mixture. The reaction mixture was then stirred at 20° C. overnight. Reaction mixtures were sampled for analysis by HPLC at about 2 h, 4 h, and 1 d; the crude reaction mixture was quenched by 95% TFA, 2.5% TIPS, 2.5% H₂O for analysis. Conversion rates and selectivity are expressed relative to Fragment 2B in Table XXA and Table XXB, respectively. Use of MeCN as a solvent for the coupling reaction resulted in a slurry (Table XXA). Conversion rates may have been impacted by pipetting variations due to the small volume (10 μL) of PyBOP stock solution used.

TABLE XXA

| Experiment No. | Solvent | Homogeneous |
|---|---|---|
| 1 | DMF | Y |
| 2 | DMSO | Y |
| 3 | THE | Partial |
| 4 | MeCN | N |
| 5 | DMI | Y |

TABLE XXB

| Experiment | 2 h | 4 h | 1 d |
|---|---|---|---|
| No. | Conversion (%) | Conversion (%) | Conversion (%) |
| 1 | 78 | 78 | 78 |
| 2 | 44 | 44 | 45 |
| 3 | 56 | 59 | 58 |
| 4 | 58 | 61 | 62 |
| 5 | 82 | 83 | 84 |

TABLE XXC

| Experiment No. | 2 h | 4 h | 1 d |
|---|---|---|---|
| 1 | 99:1 | >99:1 | >99:1 |
| 2 | >99:1 | >99:1 | >99:1 |
| 3 | 95:5 | 95:5 | 99:1 |
| 4 | 97:3 | 97:3 | 97:3 |
| 5 | 98:2 | 99:1 | 99:1 |

### Example 22: Further Screening of Solvent Conditions for Coupling Protected Variants of Fragment 1 and Fragment 2B with PyBOP/2,4,6-Collidine

To a stirred solution comprising a protected variant of Fragment 1 (Boc-His (Trt)-Aib-Glu(OtBu)-Gly-Thr(tBu)-Phe-Thr(tBu)-Ser(tBu)-Asp(OMpe)-Tyr(tBu)-Ser(tBu)-Ser(Psi(Me,Me)Pro)-OH (SEQ ID NO: 84), 1.1 eq., 0.012 mmol, 100% (w/w)) in 4 mL DMF, DMSO, THF, or DMI was added PyBOP (0.9 eq.; 0.0094 mmol; 100% (w/w)). The resulting solution was stirred at 20° C. for 30 min to activate the protected variant of Fragment 1. Then a solution comprising a protected variant of Fragment 2B (H-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 30), 200 mg, 1 eq., 0.01 mmol, 100% (w/w) in the same solvent (3 mL) was added to the activated mixture. The reaction mixture was then stirred at 20° C. for 18 h. Water (7 mL) was slowly added into the reaction mixture and stirred at 20° C. for 18 h. Each slurry was then filtered, washed with water (2 mL) twice then heptane (2 mL) twice, and dried under vacuum overnight to provide solid product. Because PyBOP did not go into solution when THF was used as a solvent (Table XXIA), DMF (0.4 mL) was added to the THF mixture. Additionally, the solution comprising THF and DMF did not result in complete conversion, and the solution was not carried forward for isolation. Reaction mixtures were sampled for analysis by HPLC at 30 min and 1 d; the crude reaction mixture was quenched by 95% TFA, 2.5% TIPS, 2.5% H₂O for analysis. Conversion rates and selectivity are expressed relative to Fragment 2B in Table XXIA and Table XXIB, respectively.

TABLE XXIA

| Experiment | | | Crude |
|---|---|---|---|
| No. | Solvent | Homogeneous | Yield |
| 1 | DMF | Y | 442 mg (>99%) |
| 2 | DMSO | Y | 388 mg (92%) |
| 3 | THF | Partial | — |
| 4 | DMI | Y | 403 mg (96%) |

TABLE XXIB

| | 30 min | 1 d | Isolated Solid |
|---|---|---|---|
| Experiment | Conversion | Conversion | Conversion |
| No. | (%) | (%) | (%) |
| 1 | 87% | 93% | 92% |
| 2 | 83% | 89% | 93% |
| 3 | 38% | 66% | — |
| 4 | 84% | 94% | 94% |

TABLE XXIC

| Experiment | | | Isolated |
|---|---|---|---|
| No. | 30 min | 1 dt | Solid |
| 1 | 96:4 | 98:2 | 99:1 |
| 2 | 90:10 | 96:4 | 95:5 |
| 3 | 98:2 | 97:3 | — |
| 4 | 96:4 | 93:7 | 92:8 |

### Example 23: Coupling Protected Variants of Fragment 1 and Fragment 2B with PyBOP/2,4,6-Collidine at 1 L Scale

To a stirred solution comprising a protected variant of Fragment 1 (Boc-His (Trt)-Aib-Glu(OtBu)-Gly-Thr(tBu)-Phe-Thr(tBu)-Ser(tBu)-Asp(OMpe)-Tyr(tBu)-Ser(tBu)-Ser(Psi(Me,Me)Pro)-OH (SEQ ID NO: 84), 1.1 eq., 4.97 mmol, 100% (w/w)) in DMI (98% (w/w); 20V) was added PyBOP (1 eq.; 4.5 mmol; 100% (w/w)) in DMI (98% (w/w); 2V). The resulting solution was stirred at 20° C. for about 1 h to activate the protected variant of Fragment 1. Then a solution comprising a protected variant of Fragment 2B (H-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 30), 4.5 mmol, 1 eq., 100% (w/w) in DMI (10V) was added to the activated mixture, with DMI (3V) used to rinse the vessel that contained the Fragment 2B solution. The reaction mixture was then stirred at 20° C. for 2 h in a 1 L reactor. Water (100% (w/w); 35V) was then added into the reaction mixture and stirred at 20° C. overnight. The slurry was then filtered, washed with water (15V) twice then heptane (15V) twice, and dried under vacuum overnight to provide solid product. The reaction mixture was sampled for analysis by HPLC at about 30 min, 2 h, and 1 d; the crude reaction mixture was quenched by 95% TFA, 2.5% TIPS, 2.5% H₂O for analysis. Conversion rates and selectivity are expressed relative to Fragment 2B in Table XXIIA and Table XXIIB, respectively.

TABLE XXIIA

| 1 h | 2 h | 1 d | Product |
|---|---|---|---|
| 90% | 90% | 92% | 92% |

TABLE XXIIB

| 1 h | 2 h | 1 d | Product |
|---|---|---|---|
| 94:6 | 96:4 | 97:3 | 95:5 |

### Example 24: Coupling Protected Variants of Fragment 1 and Fragment 2B (C18 TAG) with PyBOP

To a stirred solution comprising a protected variant of Fragment 1 (Boc-His (Trt)-Aib-Glu(OtBu)-Gly-Thr(tBu)-Phe-Thr(tBu)-Ser(tBu)-Asp(OMpe)-Tyr(tBu)-Ser(tBu)-Ser(Psi(Me,Me)Pro)-OH (SEQ ID NO: 84), 1 eq., 0.011 mmol, 100% (w/w)) was added PyBOP (1 eq.; 0.011 mmol; 100% (w/w). The resulting solution was stirred at 20° C. to activate the protected variant of Fragment 1. Then a solution comprising a protected variant of Fragment 2B in which the C-terminus is modified with a C18 (3,4,5-5ris(octadecyloxy)benzyl alcohol) TAG (Tyr(tBu)-Leu-Glu(OtBu)-Glu(OtBu)-Gln(Trt)-Ala-Ala-Lys(Boc)-Glu(OtBu)-Phe-Ile, wherein the C-terminus is modified with a C18 TAG), 0.011 mmol, 1 eq., 100% (w/w)) was added to the activated mixture. The reaction mixture was then stirred at 20° C. for 30 min and subsequently quenched by TFA (50 μL). The reaction mixture was sampled for analysis by HPLC; the crude reaction mixture was quenched by 95% TFA, 2.5% TIPS, 2.5% H₂O for analysis. Conversion rates are expressed relative to Fragment 2B in Table XXIII. The protected variant of Fragment 2B with a C18 TAG exhibited poor solubility in DMF and THF, forming a gel when mixed with DMF.

TABLE XXIII

| Experiment | Fragment 1 | Fragment 2B | Conversion | |
|---|---|---|---|---|
| No. | Solvent | Solvent | (%) | D.R. |
| 1 | DMF | DMF (gel) | 71 | 99:1 |
| 2 | DMF | THF | 85 | 99:1 |
| 3 | THF | THF | 74 | 99:1 |

### Example 25: Coupling Protected Variants of Fragment 1 and Fragment 2B (C18 TAG) with PyBOP

To a stirred solution comprising a protected variant of Fragment 1 (Boc-His (Trt)-Aib-Glu(OtBu)-Gly-Thr(tBu)-Phe-Thr(tBu)-Ser(tBu)-Asp(OMpe)-Tyr(tBu)-Ser(tBu)-Ser(Psi(Me,Me)Pro)-OH (SEQ ID NO: 84), 1.2 eq., 0.01 mmol, 100% (w/w) in DMF (100% (w/w)) was added PyBOP (1.2 eq.; 0.012 mmol; 100% (w/w)). The resulting solution was stirred at 20° C. to activate the protected variant of Fragment 1. Then a solution comprising a protected variant of Fragment 2B in which the C-terminus is modified with a C18 (3,4,5-5ris(octadecyloxy)benzyl alcohol) TAG (Tyr(tBu)-Leu-Glu(OtBu)-Glu(OtBu)-Gln(Trt)-Ala-Ala-Lys(Boc)-Glu(OtBu)-Phe-Ile, wherein the C-terminus is modified with a C18 TAG), 0.0086 mmol, 1 eq., 100% (w/w)) was added to the activated mixture. The reaction mixture was then stirred at 20° C. for 30 min and subsequently quenched by TFA (50 μL). The reaction mixture was sampled for analysis by HPLC; the crude reaction mixture was quenched by 95% TFA, 2.5% TIPS, 2.5% H₂O for analysis. Conversion rates are expressed relative to Fragment 2B in TABLE XXIV. Selectivity was >99:1 for the tested conditions.

TABLE XXIV

| Experiment | Fragment 1 | Fragment 2 | 1 h | 2 h | 4 h | 20 h |
|---|---|---|---|---|---|---|
| No. | Solvent | Solvent | Conversion | Conversion | Conversion | Conversion |
| 1 | DMF (25 V) | THF (25 V) | 94% | 95% | — | 99% |
| 2 | DMF (15 V) | MeTHF (35 V) | 89% | 91% | 90% | 97% |

Those skilled in the art will understand that the fragments used in the convergent synthetic strategy for solid phase peptide synthesis Examples 1-5 may also be used for a liquid phase peptide synthesis of Peptide A. Similarly, the fragments used in the convergent synthetic strategy for liquid phase peptide synthesis Examples 6-9, 12 and 13 may also be used for a solid phase peptide synthesis. In addition, in some embodiments of the present disclosure directed to methods comprising multiple coupling steps, a hybrid LPPS/SPPS approach can be employed (e.g., one or more coupling steps may be performed under liquid phase peptide synthesis conditions and one or more coupling steps may be performed under solid phase peptide synthesis conditions).

### OTHFR EMBODIMENTS

Provided herein as Embodiment A.1 is a peptide having a sequence of His-Aib-Glu-Gly-Thr-Phe-Thr-Ser-Asp-Tyr-Ser-Ser (SEQ ID NO: 1) (“Fragment 1”).

Provided herein as Embodiment A.2 is a protected variant of the peptide of Embodiment A. 1, wherein at least one of the His, Glu, Thr, Ser, Asp, and Tyr residues is protected with a protecting group.

Provided herein as Embodiment A.3 is the protected variant of Embodiment A.2, wherein the His residue is protected with a Boc or Fmoc protecting group.

Provided herein as Embodiment A.4 is the protected variant of Embodiment A.3, wherein the N-terminal His is protected with a Boc protecting group.

Provided herein as Embodiment A.5 is the protected variant of any one of Embodiments A.2 to A.4, wherein the Glu residue at position 3 is protected as a t-butyl ester (OtBu).

Provided herein as Embodiment A.6 is the protected variant of any one of Embodiments A.2 to A.5, wherein the Thr residue at position 5 is protected as a t-butyl ether (tBu).

Provided herein as Embodiment A.7 is the protected variant of any one of Embodiments A.2 to A.6, wherein the Thr residue at position 7 is protected as a t-butyl ether (tBu).

Provided herein as Embodiment A.8 is the protected variant of any one of Embodiments A.2 to A.7, wherein the Ser residue at position 8 is protected as a t-butyl ether (tBu).

Provided herein as Embodiment A.9 is the protected variant of any one of Embodiments A.2 to A.8, wherein the Ser residue at position 11 is protected as a t-butyl ether (tBu).

Provided herein as Embodiment A. 10 is the protected variant of any one of Embodiments A.2 to A.9, wherein the Ser residue at position 12 is protected as a pseudoproline moiety (psiMe, Mepro).

Provided herein as Embodiment A. 11 is the protected variant of any one of Embodiments A.2 to A. 10, wherein the Asp residue at position 9 is protected as a t-butyl ester (OtBu).

Provided herein as Embodiment A. 12 is the protected variant of any one of Embodiments A.2 to A. 11, wherein the Tyr residue at position 10 is protected as a t-butyl ether (tBu).

Provided herein as Embodiment A. 13 is a protected variant of the peptide of Embodiment A.1, having a structure of

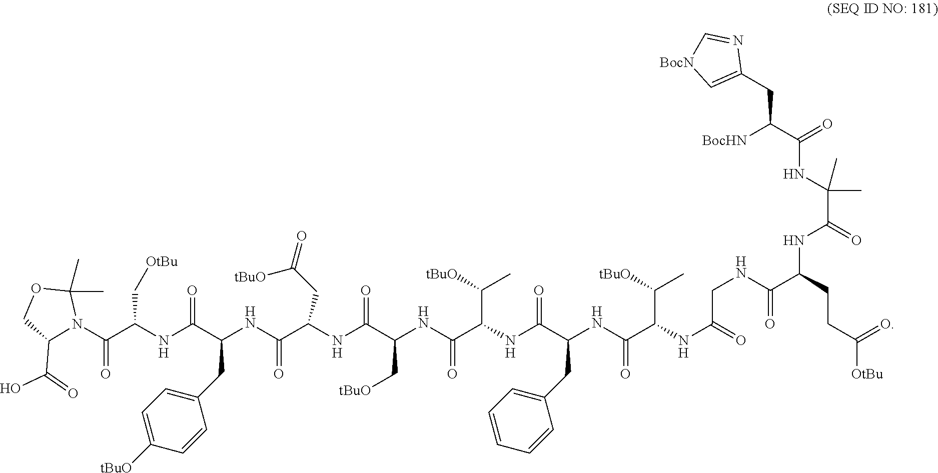 [SMILES: C[C@@H](OC(C)(C)C)[C@H](NC(=O)CNC(=O)[C@H](CCC(=O)OC(C)(C)C)NC(=O)C(C)(C)NC(=O)[C@H](Cc1cn(C(=O)OC(C)(C)C)cn1)NC(=O)OC(C)(C)C)C(=O)N[C@@H](Cc1ccccc1)C(=O)N[C@H](C(=O)N[C@@H](COC(C)(C)C)C(=O)N[C@@H](CC(=O)OC(C)(C)C)C(=O)N[C@@H](Cc1ccc(OC(C)(C)C)cc1)C(=O)N[C@@H](COC(C)(C)C)C(=O)N1[C@H](C(=O)O)COC1(C)C)[C@@H](C)OC(C)(C)C]

Provided herein as Embodiment A. 14 is a peptide having a sequence of His-Aib-Glu-Gly-Thr-Phe-Thr-Ser-Asp-Tyr-Ser-Ser-Tyr (SEQ ID NO: 11) (“Fragment 1A”).

Provided herein as Embodiment A. 15 is a protected variant of the peptide of Embodiment A. 14, wherein at least one of the His, Glu, Thr, Ser, Asp, and Tyr residues is protected with a protecting group.

Provided herein as Embodiment A. 16 is the protected variant of Embodiment A. 15, wherein the His residue is protected with a Boc or Fmoc protecting group.

Provided herein as Embodiment A. 17 is the protected variant of Embodiment A. 16, wherein the N-terminal His is protected with a Boc protecting group.

Provided herein as Embodiment A. 18 is the protected variant of any one of Embodiments A. 15 to A.17, wherein the Glu residue at position 3 is protected as a t-butyl ester (OtBu).

Provided herein as Embodiment A. 19 is the protected variant of any one of Embodiments A. 15 to A. 18, wherein the Thr residue at position 5 is protected as a t-butyl ether (tBu).

Provided herein as Embodiment A.20 is the protected variant of any one of Embodiments A. 15 to A. 19, wherein the Thr residue at position 7 is protected as a t-butyl ether (tBu).

Provided herein as Embodiment A.21 is the protected variant of any one of Embodiments A. 15 to A.20, wherein the Ser residue at position 8 is protected as a t-butyl ether (tBu).

Provided herein as Embodiment A.22 is the protected variant of any one of Embodiments A. 15 to A.21, wherein the Asp residue at position 9 is protected as a t-butyl ester (OtBu).

Provided herein as Embodiment A.23 is the protected variant of any one of Embodiments A. 15 to A.22, wherein the Tyr residue at position 10 is protected as a t-butyl ether (tBu).

Provided herein as Embodiment A.24 is the protected variant of any one of Embodiments A. 15 to A.23, wherein the Ser residue at position 11 is protected as a t-butyl ether (tBu).

Provided herein as Embodiment A.25 is the protected variant of any one of Embodiments A. 15 to A.24, wherein the Ser residue at position 12 is protected as a t-butyl ether (tBu).

Provided herein as Embodiment A.26 is the protected variant of any one of Embodiments A. 15 to A.25, wherein the Tyr at position 13 is protected as a t-butyl ether (tBu).

Provided herein as Embodiment A.27 is a protected variant of the peptide of Embodiment A. 14, having a structure of

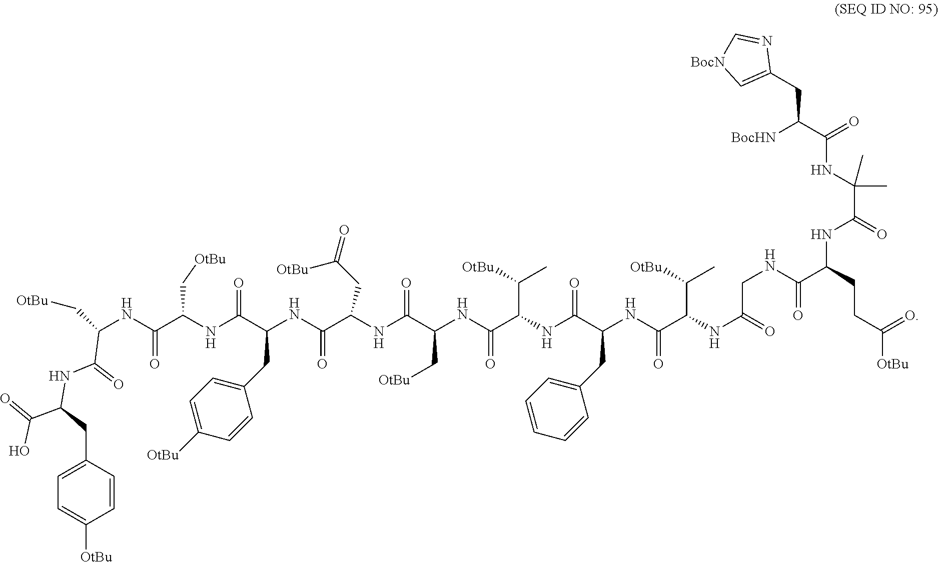 [SMILES: C[C@@H](OC(C)(C)C)[C@H](NC(=O)CNC(=O)[C@H](CCC(=O)OC(C)(C)C)NC(=O)C(C)(C)NC(=O)[C@H](Cc1cn(C(=O)OC(C)(C)C)cn1)NC(=O)OC(C)(C)C)C(=O)N[C@@H](Cc1ccccc1)C(=O)N[C@H](C(=O)N[C@@H](COC(C)(C)C)C(=O)N[C@@H](CC(=O)OC(C)(C)C)C(=O)N[C@@H](Cc1ccc(OC(C)(C)C)cc1)C(=O)N[C@@H](COC(C)(C)C)C(=O)N[C@@H](COC(C)(C)C)C(=O)N[C@@H](Cc1ccc(OC(C)(C)C)cc1)C(=O)O)[C@@H](C)OC(C)(C)C]

Provided herein as Embodiment A.28 is a peptide having a sequence of Tyr-Leu-Glu-Glu-Gln-Ala-Ala-Lys-Glu-Phe-Ile-Ala (SEQ ID NO: 2) (“Fragment 2”).

Provided herein as Embodiment A.29 is a protected variant of the peptide of Embodiment A.28, wherein at least one of the Tyr, Glu, Gln, and Lys residues is protected with a protecting group.

Provided herein as Embodiment A.30 is the protected variant of Embodiment A.29, wherein the Tyr residue at position 1 is protected as a t-butyl ether (tBu).

Provided herein as Embodiment A.31 is the protected variant of Embodiment A.29 or A.30, wherein the Tyr at position 1 is protected with an Fmoc protecting group at the N-terminus.

Provided herein as Embodiment A.32 is the protected variant of any one of Embodiments A.29 to A.31, wherein the Glu residue at position 3 is protected as a t-butyl ester (OtBu).

Provided herein as Embodiment A.33 is the protected variant of any one of Embodiments A.29 to A.32, wherein the Gln residue at position 4 is protected as a triphenylmethyl amide (Trt).

Provided herein as Embodiment A.34 is the protected variant of any one of Embodiments A.29 to A.33, wherein the Lys at position 7 is protected as a t-butyloxy carbonyl amide (Boc).

Provided herein as Embodiment A.35 is the protected variant of any one of Embodiments A.29 to A.34, wherein the Glu at position 8 is protected as a t-butyl ester (OtBu).

Provided herein as Embodiment A.36 is a protected variant of the peptide of Embodiment A.28, having a structure of

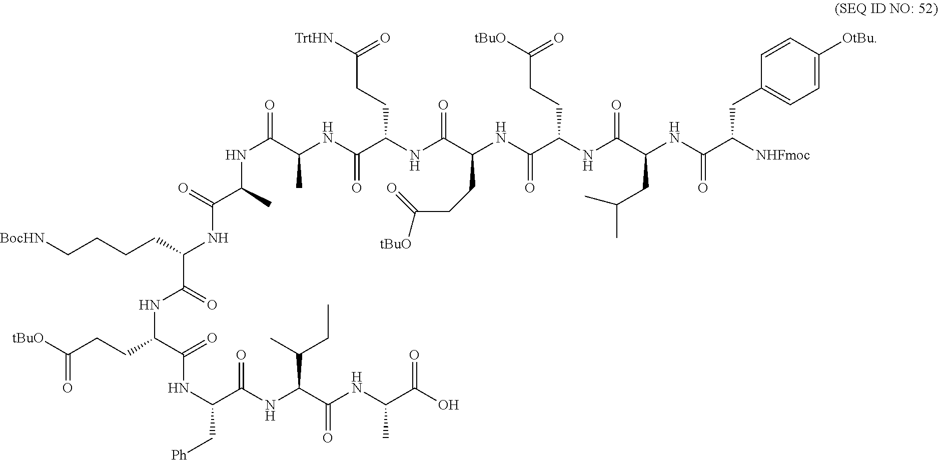

Provided herein as Embodiment A.37 is a peptide having a sequence of Leu-Glu-Glu-Gln-Ala-Ala-Lys-Glu-Phe-Ile-Ala (SEQ ID NO: 12) (“Fragment 2A”).

Provided herein as Embodiment A.38 is a protected variant of the peptide of Embodiment A.37, wherein at least one of the Glu, Gln, and Lys residues is protected with a protecting group.

Provided herein as Embodiment A.39 is the protected variant of Embodiment A.38, wherein the Glu residue at position 2 is protected as a t-butyl ester (OtBu).

Provided herein as Embodiment A.40 is the protected variant of Embodiment A.38 or A.39, wherein the Glu residue at position 3 is protected as a t-butyl ester (OtBu).

Provided herein as Embodiment A.41 is the protected variant of any one of Embodiments A.38 to A.40, wherein the Gln residue at position 4 is protected as a triphenylmethyl amide (Trt).

Provided herein as Embodiment A.42 is the protected variant of any one of Embodiments A.38 to A.41, wherein the Lys at position 7 is protected as a t-butyloxy carbonyl amide (Boc).

Provided herein as Embodiment A.43 is the protected variant of any one of Embodiments A.38 to A.42, wherein the Glu at position 8 is protected as a t-butyl ester (OtBu).

Provided herein as Embodiment A.44 is a protected variant of the peptide of Embodiment A.37, having a structure of

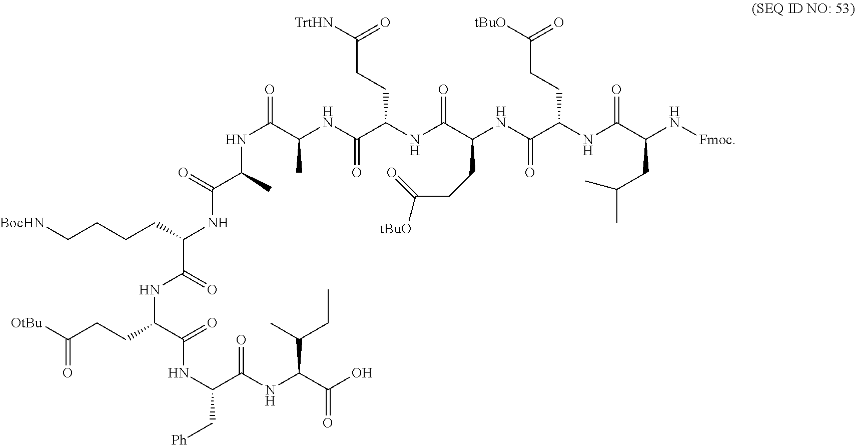

Provided herein as Embodiment A.45 is a peptide having a sequence of Trp-Leu-Val-Lys-Gly-Gly-Gly-Gly-Gly-Gly-Gly (SEQ ID NO: 3) (“Fragment 3”).

Provided herein as Embodiment A.46 is a protected variant of the peptide of Embodiment A.45, wherein at least one of the Trp and Lys residues is protected with a protecting group.

Provided herein as Embodiment A.47 is the protected variant of Embodiment A.46, wherein the Trp is protected with an Fmoc protecting group at the N-terminus.

Provided herein as Embodiment A.48 is the protected variant of Embodiment A.46, wherein the Trp is protected as a t-butyloxy carbonyl amide (Boc) at the Trp side chain.

Provided herein as Embodiment A.49 is the protected variant of any one of Embodiments A.46 to A.48, wherein the Lys residue at position 4 is protected as t-butyloxy carbonyl amide (Boc).

Provided herein as Embodiment A.50 is a protected variant of the peptide of Embodiment A.45, having a structure of

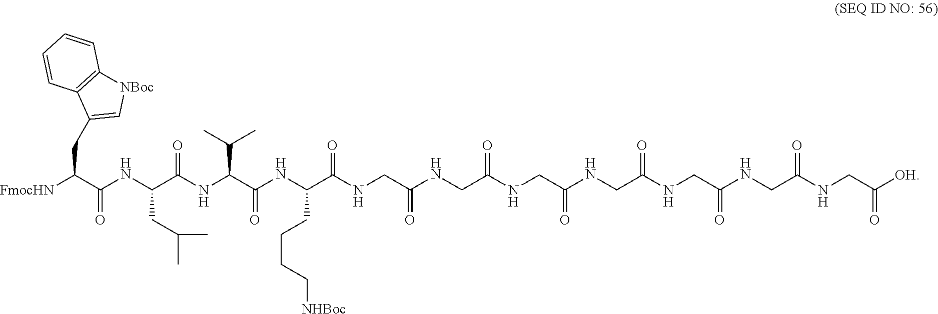

Provided herein as Embodiment A.51 is a peptide having a sequence of Ala-Trp-Leu-Val-Lys-Gly-Gly-Gly-Gly-Gly-Gly-Gly (SEQ ID NO: 13) (“Fragment 3A”).

Provided herein as Embodiment A.52 is a protected variant of the peptide of Embodiment A.51, wherein at least one of the Ala, Trp and Lys residues is protected with a protecting group.

Provided herein as Embodiment A.53 is the protected variant of Embodiment A.52, wherein the Ala is protected with an Fmoc protecting group at the N-terminus.

Provided herein as Embodiment A.54 is the protected variant of Embodiment A.52 or A.53, wherein the Trp is protected as a t-butyloxy carbonyl amide (Boc) at the Trp side chain.

Provided herein as Embodiment A.55 is the protected variant of any one of Embodiments A.52 to A.54, wherein the Lys residue at position 4 is protected as t-butyloxy carbonyl amide (Boc).

Provided herein as Embodiment A.56 is a protected variant of the peptide of Embodiment A.51, having a structure of

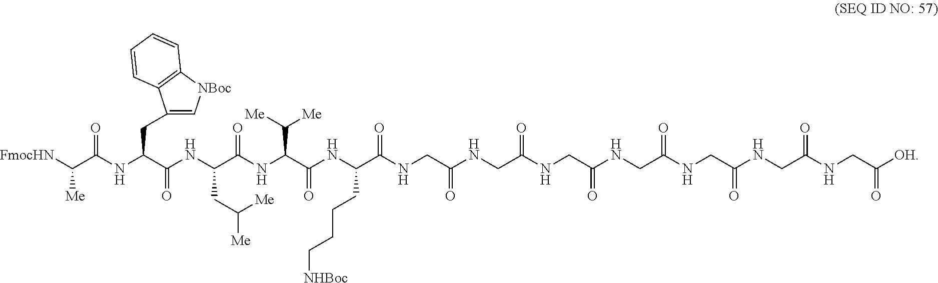

Provided herein as Embodiment A.57 is a peptide having a sequence of Ser-Gly-Gly-Gly-Gly-Ser-Gly-Gly-Gly-Gly-Ser-Lys (SEQ ID NO: 4) (“Fragment 4”).

Provided herein as Embodiment A.58 is a protected variant of the peptide of Embodiment A.57, wherein at least one of the Ser and Lys residues is protected with a protecting group.

Provided herein as Embodiment A.59 is the protected variant of Embodiment A.58, wherein the Ser residue at position 1 is protected with an Fmoc protecting group at the N-terminus.

Provided herein as Embodiment A.60 is the protected variant of Embodiment A.58 or A.59, wherein the Ser residue at position 1 is protected as a t-butyl ether (tBu) on the Ser side chain.

Provided herein as Embodiment A.61 is the protected variant of any one of Embodiments A.58 to A.60, wherein the Ser residue at position 6 is protected as a t-butyl ether (tBu).

Provided herein as Embodiment A.62 is the protected variant of any one of Embodiments A.58 to A.61, wherein the Ser residue at position 11 is protected as a t-butyl ether (tBu).

Provided herein as Embodiment A.63 is the protected variant of any one of Embodiments A.58 to A.62, wherein the Lys residue at position 12 is protected as a N-ε-1-(4,4-dimethyl-2,6-dioxocyclohex-1-ylidene)-3-methylbutyl amine (ivDde).

Provided herein as Embodiment A.64 is a conjugate of the peptide of Embodiment A.57 or the protected variant of any one of Embodiments A.58 to A.63, wherein the Lys residue at position 12 is coupled to a solid support.

Provided herein as Embodiment A.65 is the conjugate of Embodiment A.64, wherein the solid support is a resin material.

Provided herein as Embodiment A.66 is the conjugate of Embodiment A.65, wherein the resin material is a swellable resin material.

Provided herein as Embodiment A.67 is the conjugate of Embodiment A.66, wherein the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing.

Provided herein as Embodiment A.68 is the conjugate of any one of Embodiments A.64 to A.67, further comprising a linker through which the peptide or the protected variant is attached to the solid support.

Provided herein as Embodiment A.69 is the conjugate of Embodiment A.68, wherein the linker is a rink-amide linker or a Sieber-amide linker.

Provided herein as Embodiment A.70 is the conjugate of any one of Embodiments A.57 or A.64 to A.69, having a structure of

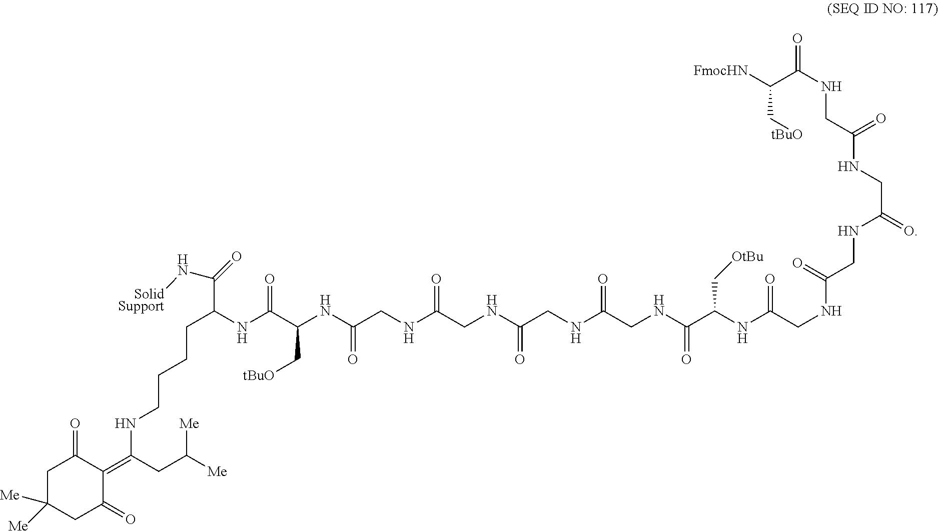

Provided herein as Embodiment A.71 is a peptide having a sequence of Ala-Trp-Leu-Val-Lys-Gly-Gly-Gly (SEQ ID NO: 5) (“Fragment 5”).

Provided herein as Embodiment A.72 is a protected variant of the peptide of Embodiment A.71, wherein at least one of the Ala, Trp and Lys residues is protected with a protecting group.

Provided herein as Embodiment A.73 is the protected variant of Embodiment A.72, wherein the Ala residue at position 1 is protected with an Fmoc protecting group at the N-terminus.

Provided herein as Embodiment A.74 is the protected variant of Embodiments A.72 or A.73, wherein the Trp residue at position 2 is protected as t-butyloxy carbonyl amide (Boc).

Provided herein as Embodiment A.75 is the protected variant of any one of Embodiments A.72 to A.74, wherein the Lys residue at position 5 is protected as t-butyloxy carbonyl amide (Boc).

Provided herein as Embodiment A.76 is a protected variant of the peptide of Embodiment A.71, having a structure of

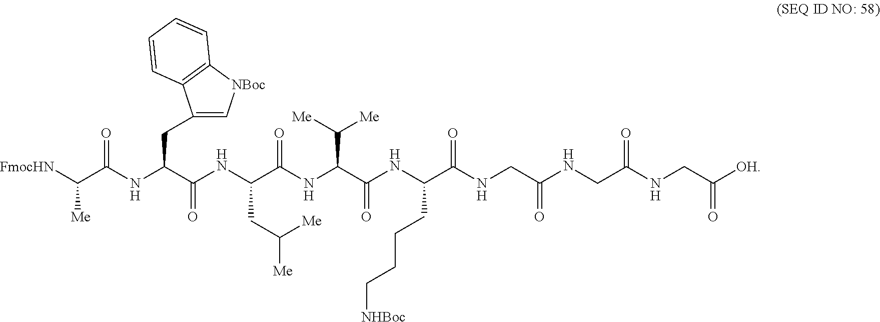

Provided herein as Embodiment A.77 is a peptide having a sequence of Gly-Gly-Gly-Gly-Ser-Gly-Gly-Gly-Gly-Ser-Gly-Gly-Gly-Gly-Ser-Lys (SEQ ID NO: 6) (“Fragment 6”).

Provided herein as Embodiment A.78 is a protected variant of the peptide of Embodiment A.77, wherein at least one of the Ser and Lys is protected with a protecting group.

Provided herein as Embodiment A.79 is the protected variant of Embodiment A.78, wherein the Gly residue at position 1 is protected with an Fmoc protecting group at the N-terminus.

Provided herein as Embodiment A.80 is the protected variant of Embodiment A.78 or A.79, wherein the Ser residue at position 5 is protected as a t-butyl ether (tBu).

Provided herein as Embodiment A.81 is the protected variant of any one of Embodiments A.78 to A.80, wherein the Ser residue at position 10 is protected as a t-butyl ether (tBu).

Provided herein as Embodiment A.82 is the protected variant of any one of Embodiments A.78 to A.81, wherein the Ser residue at position 15 is protected as a t-butyl ether (tBu).

Provided herein as Embodiment A.83 is the protected variant of any one of Embodiments A.78 to A.82, wherein the Lys residue at position 16 is protected as a N-ε-1-(4,4-dimethyl-2,6-dioxocyclohex-1-ylidene)-3-methylbutyl amine (ivDde).

Provided herein as Embodiment A.84 is a conjugate of the peptide of Embodiment A.77 or the protected variant of any one of Embodiments A.78 to A.83, wherein the Lys residue at position 12 is coupled to a solid support.

Provided herein as Embodiment A.85 is the conjugate of Embodiment A.84, wherein the solid support is a resin material.

Provided herein as Embodiment A.86 is the conjugate of Embodiment A.84 or A.85, wherein the resin material is a swellable resin material.

Provided herein as Embodiment A.87 is the conjugate of any one of Embodiments A.84 to A.86, wherein the swellable resin material comprises a polystyrene bead, low cross-linked polystyrene bead, mixed block polystyrene-divinylbenzene bead, mixed block polystyrene-polyethylene glycol bead, polyacrylamide polyethylene glycol copolymer bead, or a combination of any of the foregoing.

Provided herein as Embodiment A.88 is the conjugate of Embodiment A.85, wherein the resin material is Sieber resin, rink resin, or xanthenyl resin.

Provided herein as Embodiment A.89 is a conjugate of a protected variant of the peptide of Embodiment A.77, having a structure of

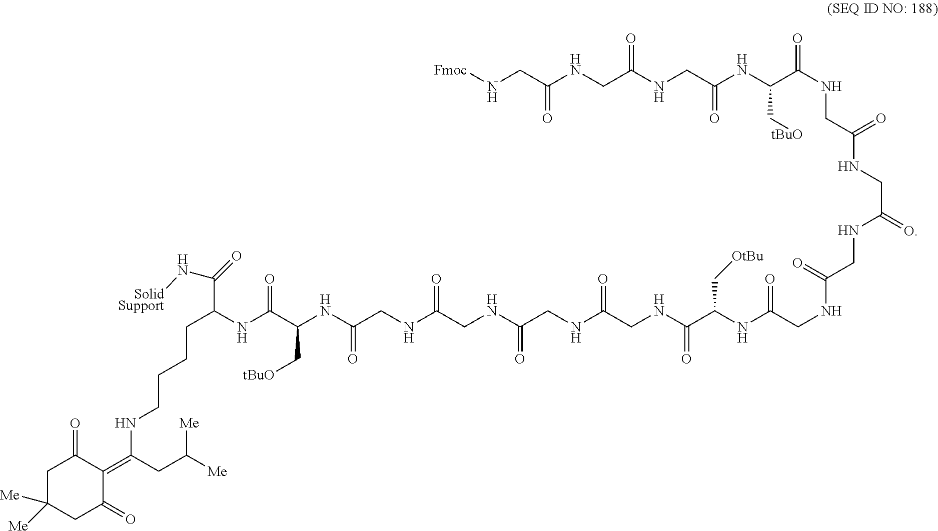

Provided herein as Embodiment A.90 is a method of preparing Peptide A having a sequence of His-Aib-Glu-Gly-Thr-Phe-Thr-Ser-Asp-Tyr-Ser-Ser-Tyr-Leu-Glu-Glu-Gln-Ala-Ala-Lys-Glu-Phe-Ile-Ala-Trp-Leu-Val-Lys-Gly-Gly-Gly-Gly-Gly-Gly-Gly-Ser-Gly-Gly-Gly-Gly-Ser-Gly-Gly-Gly-Gly-Ser-Lys (SEQ ID NO: 7) (“Peptide A”), or a protected variant thereof, comprising: (A) (i) (a) reacting a protected variant of Fragment 3 of any one of Embodiments A.46 to A.50 with a protected variant of Fragment 4 of any one of Embodiments A.58 to A.70 under solid phase peptide coupling conditions to form a protected variant of Peptide E (SEQ ID NO: 8), wherein the protected variant of Fragment 4 is on solid support before the reacting and the protected variant of Peptide E is on solid support after the reacting; and (b) reacting the protected variant of Peptide E on solid support from step (A) (i) (a) with a protected alanine under solid phase peptide coupling conditions to form a protected variant of Peptide B (SEQ ID NO: 9), wherein the protected variant of Peptide B is on solid support after the reacting; OR (ii) reacting a protected variant of Fragment 3A of any one of Embodiments A.52 to A.56 with a protected variant of Fragment 4 of any one of Embodiments A.58 to A.70 under solid phase peptide coupling conditions to form a protected variant of Peptide B (SEQ ID NO: 9), wherein the protected variant of Fragment 4 is on solid support before the reacting and the protected variant of Peptide B is on solid support after the reacting; OR (iii) reacting a protected variant of Fragment 5 of any one of Embodiments A.72 to A.76 with a protected variant of Fragment 6 of any one of Embodiments A.78 to A.89 under solid phase peptide coupling conditions to form a protected variant of Peptide B (SEQ ID NO: 9), wherein the protected variant of Fragment 6 is on solid support before the reacting and the protected variant of Peptide B is on solid support after the reacting; (B) reacting a protected variant of Fragment 2A of any one of Embodiments A.38 to A.43 with the protected variant of Peptide B on solid support from step (A) under solid phase peptide coupling conditions to form a protected variant of Peptide C (SEQ ID NO: 10), wherein the protected variant of Peptide C is on solid support after the reacting; (C) reacting a protected variant of Fragment 1A of any one of Embodiments A. 15 to A.27 with the protected variant of Peptide C on solid support from step (B) under solid phase peptide coupling conditions to form a protected variant of Peptide A, wherein the protected variant of Peptide A is on solid support after the reacting; and (D) optionally deprotecting and cleaving the protected variant of Peptide A from the solid support to isolate Peptide A.

Provided herein as Embodiment A.91 is the method of Embodiment A.90, wherein the solid phase peptide coupling conditions of any one of steps (A), (B), and (C) comprise a coupling reagent in DMF or DMSO at a temperature in the range of 20° C. to 50° C.

Provided herein as Embodiment A.92 is the method of Embodiment A.91, wherein the coupling reagent comprises DIC, TBTU, HATU, PyBOP, PyOxim, 6-Cl—HOBt, HCTU, or a combination of any of the foregoing.

Provided herein as Embodiment A.93 is the method of Embodiment A.91 or A.92, wherein the solid phase peptide coupling conditions further comprise a base.

Provided herein as Embodiment A.94 is the method of Embodiment A.93, wherein the base comprises collidine, triethylamine, diisopropylethylamine, or a combination of any of the foregoing.

Provided herein as Embodiment A.95 is the method of any one of Embodiments A.90 to A.94, wherein the solid phase coupling conditions of step (A) (i) (a), A (ii), or A (iii) comprise TBTU and collidine in DMSO at 20° C.

Provided herein as Embodiment A.96 is the method of any one of Embodiments A.90 to A.95, wherein the solid phase coupling conditions of step (B) comprise DIC/Oxyma in DMF at 50° C.

Provided herein as Embodiment A.97 is the method of any one of Embodiments A.90 to A.96, wherein the solid phase coupling conditions of step (C) comprise DIC/Oxyma in DMF at 20° C.

Provided herein as Embodiment A.98 is the method of any one of Embodiments A.90 to A.97, wherein the deprotecting of step (D) comprises mixing Peptide A (before or after cleaving from solid support) with an aqueous solution of trifluoroacetic acid and triisopropyl silane.

Provided herein as Embodiment A.99 is the method of any one of Embodiments A.90 to A.98, wherein the deprotecting of step (D) comprises mixing Peptide A (before or after cleaving from solid support) with hydrazine.

Provided herein as Embodiment A. 100 is the method of any one of Embodiments A.90 to A.99, wherein the solid support is an amide-based resin.

Provided herein as Embodiment A. 101 is the method of Embodiment A. 100, wherein the amide-based resin is rink amide resin or xanthenyl resin.

Provided herein as Embodiment A. 102 is the method of any one of Embodiments A.90 to A. 101, wherein the cleaving of step (D) comprises mixing the peptide with trifluoroacetic acid: triisopropylsilane:water in a 95:2.5:2.5 ratio.

Provided herein as Embodiment A. 103 is the method of any one of Embodiments A.90 to A. 102, further comprising adding a bromoacetyl moiety to the Lys at the C-terminus of Peptide A.

Provided herein as Embodiment B.1 is a method of preparing Peptide B or a protected variant thereof, comprising coupling a protected variant of Fragment 5 (“carboxyl coupling partner”) with a protected variant of Fragment 6 (“amine coupling partner”) to form a protected variant of Peptide B, wherein Peptide B comprises the amino acid sequence of SEQ ID NO: 9, Fragment 5 comprises the amino acid sequence of SEQ ID NO: 5, and Fragment 6 comprises the amino acid sequence of SEQ ID NO: 6.

Provided herein as Embodiment B.2 is a method of preparing Peptide B or a protected variant thereof, comprising coupling a protected variant of Fragment 3A (“carboxyl coupling partner”) with a protected variant of Fragment 4 (“amine coupling partner”) to form a protected variant of Peptide B, wherein Peptide B comprises the amino acid sequence of SEQ ID NO: 9, Fragment 3A comprises the amino acid sequence of SEQ ID NO: 13, and Fragment 4 comprises the amino acid sequence of SEQ ID NO: 4.

Provided herein as Embodiment B.3 is a method of preparing Peptide E or a protected variant thereof, comprising coupling a protected variant of Fragment 3 (“carboxyl coupling partner”) with a protected variant of Fragment 4 (“amine coupling partner”) to form a protected variant of Peptide E, wherein Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8.

Provided herein as Embodiment B.4 is a method of preparing Peptide B or a protected variant thereof, comprising coupling a protected variant of Peptide E (“amine coupling partner”) with an N-protected alanine (“carboxyl coupling partner”) to form a protected variant of Peptide B, wherein Peptide E comprises the amino acid sequence of SEQ ID NO: 8 and Peptide B comprises the amino acid sequence of SEQ ID NO: 9.

Provided herein as Embodiment B.5 is a method of preparing Peptide D or a protected variant thereof, comprising coupling a protected variant of Fragment 1 (“carboxyl coupling partner”) with a protected variant of Fragment 2B (“amine coupling partner”) in to form a protected variant of Peptide D, wherein Peptide D comprises the amino acid sequence of SEQ ID NO: 15, Fragment 1 comprises the amino acid sequence of SEQ ID NO: 1, and Fragment 2B comprises the amino acid sequence of SEQ ID NO: 16.

Provided herein as Embodiment B.6 is a method of preparing Peptide A or a protected variant thereof, comprising coupling a protected variant of Peptide B (“amine coupling partner”) with a protected variant of Peptide D (“carboxyl coupling partner”) to form a protected variant of Peptide A, wherein Peptide B comprises the amino acid sequence of SEQ ID NO: 9, Peptide D comprises the amino acid sequence of SEQ ID NO: 15, and Peptide A comprises the amino acid sequence of SEQ ID NO: 7.

Provided herein as Embodiment B.7 is a method of preparing Peptide C or a protected variant thereof, comprising coupling a protected variant of Fragment 2A (“carboxyl coupling partner”) with a protected variant of Peptide B (“amine coupling partner”) to form a protected variant of Peptide C, wherein Fragment 2A comprises the amino acid sequence of SEQ ID NO: 12, Peptide B comprises the amino acid sequence of SEQ ID NO: 9, and Peptide C comprises the amino acid sequence of SEQ ID NO: 10.

Provided herein as Embodiment B.8 is a method of preparing Peptide C or a protected variant thereof, comprising coupling a protected variant of Fragment 2C (“carboxyl coupling partner”) with a protected variant of Peptide E (“amine coupling partner”) to form a protected variant of Peptide C, wherein Fragment 2C comprises the amino acid sequence of SEQ ID NO: 17, Peptide E comprises the amino acid sequence of SEQ ID NO: 8, and Peptide C comprises the amino acid sequence of SEQ ID NO: 10.

Provided herein as Embodiment B.9 is a method of preparing Peptide A or a protected variant thereof, comprising coupling a protected variant of Fragment 1A (“carboxyl coupling partner”) with a protected variant of Peptide C (“amine coupling partner”) to form a protected variant of Peptide A, wherein Fragment 1A comprises the amino acid sequence of SEQ ID NO: 11, Peptide C comprises the amino acid sequence of SEQ ID NO: 10, and Peptide A comprises the amino acid sequence of SEQ ID NO: 7.

Provided herein as Embodiment B.10 is a method of preparing Peptide F or a protected variant thereof, comprising coupling a protected variant of Fragment 2 (“carboxyl coupling partner”) with a protected variant of Peptide E (“amine coupling partner”) to form a protected variant of Peptide F, wherein Fragment 2 comprises the amino acid sequence of SEQ ID NO: 2, Peptide E comprises the amino acid sequence of SEQ ID NO: 8, and Peptide F comprises the amino acid sequence of SEQ ID NO: 14.

Provided herein as Embodiment B.11 is a method of preparing Peptide A or a protected variant thereof, comprising coupling a protected variant of Fragment 1 (“carboxyl coupling partner”) with a protected variant of Peptide F (“amine coupling partner”) to form a protected variant of Peptide A, wherein Fragment 1 comprises the amino acid sequence of SEQ ID NO: 1, Peptide F comprises the amino acid sequence of SEQ ID NO: 14, and Peptide A comprises the amino acid sequence of SEQ ID NO: 7.

Provided herein as Embodiment B. 12 is the method of any one of Embodiments B.1 to B.11, wherein the amine coupling partner and the carboxyl coupling partner are coupled under liquid phase peptide coupling conditions.

Provided herein as Embodiment B.13 is the method of any one of Embodiments B.1 to B. 12, wherein the amine coupling partner and the carboxyl coupling partner are coupled under classical solution phase peptide coupling conditions.

Provided herein as Embodiment B.14 is the method of any one of Embodiments B.1 to B. 12, wherein the amine coupling partner and the carboxyl coupling partner are coupled under tag-assisted liquid phase peptide coupling conditions.

Provided herein as Embodiment B.15 is the method of any one of Embodiments B.1 to B.11, wherein the amine coupling partner and the carboxyl coupling partner are coupled under solid phase peptide coupling conditions.

Provided herein as Embodiment B.16 is the method of any one of Embodiments B.1 to B.15, wherein the method comprises:

contacting the carboxyl coupling partner with a coupling composition to form a carboxyl coupling partner with an activated C-terminal carboxyl group (“activated carboxyl coupling partner”); and
contacting the amine coupling partner with the activated carboxyl coupling partner, wherein the contacting does not occur in the presence of a coupling reagent.

Provided herein as Embodiment B.17 is the method of Embodiment B.16, wherein the carboxyl coupling partner is present in molar excess relative to the coupling reagent in the coupling composition during the contacting of the carboxyl coupling partner and the coupling composition.

Provided herein as Embodiment B.18 is the method of any one of Embodiments B.1 to B.15, wherein the method comprises coupling the carboxyl coupling partner with the amine coupling partner in the presence of a coupling composition.

Provided herein as Embodiment B. 19 is the method of any one of Embodiments B. 16 to B.18, wherein the coupling composition comprises a coupling reagent and a base.

Provided herein as Embodiment B.20 is the method of any one of Embodiments B. 16 to B.18, wherein the coupling composition comprises a coupling reagent and a coupling additive.

Provided herein as Embodiment B.21 is the method of any one of Embodiments B. 16 to B.19, wherein the coupling composition comprises a coupling reagent, a base, and a coupling additive.

Provided herein as Embodiment B.22 is the method of any one of Embodiments B. 19 to B.21, wherein the coupling reagent comprises a carbodiimide, an aminium (uronium) salt, a phosphonium salt, a phosphonic acid anhydride, or a phosphate-type coupling reagent.

Provided herein as Embodiment B.23 is the method of any one of Embodiments B. 19 to B.22, wherein the coupling reagent is selected from DIC, DCC, EDC, HBTU, TBTU, HATU, HCTU, TATU, TCTU, TNTU, TSTU, COMU, TOTU, PyBOP, PyAOP, PyBroP, PyCIOP, BOP, BOP—Cl, PyOxim, T3P, DEPBT, and combinations of any of the foregoing.

Provided herein as Embodiment B.24 is the method of Embodiment B.19 or Embodiment B.21, wherein the base is DIPEA, NMM, NEM, TEA, 2,6-lutidine, collidine, TMP, or a combination of any of the foregoing.

Provided herein as Embodiment B.25 is the method of Embodiment B.20 or Embodiment B.21, wherein the coupling additive is DMAP, HOBt, 6-Cl—HOBt, HOAt, Oxyma, or a combination of any of the foregoing.

Provided herein as Embodiment B.26 is the method of any one of Embodiments B. 16 to B.25, wherein the coupling composition comprises DIC/Oxyma, T3P/Oxyma, T3P/Oxyma/collidine, HCTU/collidine, HATU/collidine, HBTU/collidine, TBTU/collidine, DMAP/EDC, Oxyma/PyOxim, Oxyma/PyOxim/collidine, or PyBOP/collidine.

Provided herein as Embodiment B.27 is the method of any one of Embodiments B.1 to B.26, wherein the coupling is conducted at a temperature in the range of 10° C. to 60° C.

Provided herein as Embodiment B.28 is the method of any one of Embodiments B.1 to B.27, wherein the coupling is conducted at a temperature in the range of 20° C. to 50° C.

Provided herein as Embodiment B.29 is the method of any one of Embodiments B.1 to B.28, wherein the coupling is conducted at a temperature in the range of 35° C. to 45° C.

Provided herein as Embodiment B.30 is the method of any one of Embodiments B.1 to B.28, wherein the coupling is conducted at a temperature in the range of 20° C. to 25° C.

Provided herein as Embodiment B.31 is the method of any one of Embodiments B.1 to B.30, wherein the coupling is conducted for a time in the range of 1 hour to 72 hours.

Provided herein as Embodiment B.32 is the method of any one of Embodiments B.1 to B.31, wherein the coupling is conducted for a time in the range of 6 hours to 24 hours.

Provided herein as Embodiment B.33 is a method of preparing Peptide A or a protected variant thereof, comprising:
(A)
(i) (a) coupling or having coupled a protected variant of Fragment 3 with a protected variant of Fragment 4 to form a first protected variant of Peptide E, wherein the protected variant of Fragment 3 has a free carboxyl group at the C-terminus and a protected N-terminus, the protected variant of Fragment 4 has a free amino group at the N-terminus, Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8; (b) selectively deprotecting or having selectively deprotected the N-terminus of the first protected variant of Peptide E from step (A) (i) (a) to form a second protected variant of Peptide E with a free amino group at the N-terminus; (c) coupling or having coupled the second protected variant of Peptide E from step (A) (i) (b) with an N-protected alanine to form a first protected variant of Peptide B, wherein Peptide B comprises the amino acid sequence of SEQ ID NO: 9; and (d) selectively deprotecting or having selectively deprotected the N-terminus of the first protected variant of Peptide B from step (A) (i) (c) to form a second protected variant of Peptide B with a free amino group at the N-terminus; OR
(ii) (a) coupling or having coupled a protected variant of Fragment 3A with a protected variant of Fragment 4 to form a first protected variant of Peptide B, wherein the protected variant of Fragment 3A has a free carboxyl group at the C-terminus and a protected N-terminus, the protected variant of Fragment 4 has a free amino group at the N-terminus, Fragment 3A comprises the amino acid sequence of SEQ ID NO: 13, Fragment 4 comprises the amino acid sequence of SEQ ID NO: 4, and Peptide B comprises the amino acid sequence of SEQ ID NO: 9; and (b) selectively deprotecting or having selectively deprotected the N-terminus of the first protected variant of Peptide B from step (A) (ii) (a) to form a second protected variant of Peptide B with a free amino group at the N-terminus; OR
(iii) (a) coupling or having coupled a protected variant of Fragment 5 with a protected variant of Fragment 6 to form a first protected variant of Peptide B, wherein the protected variant of Fragment 5 has a free carboxyl group at the C-terminus and a protected N-terminus, the protected variant of Fragment 6 has a free amino group at the N-terminus, Fragment 5 comprises the amino acid sequence of SEQ ID NO: 5, Fragment 6 comprises the amino acid sequence of SEQ ID NO: 6, and Peptide B comprises the amino acid sequence of SEQ ID NO: 9; and (b) selectively deprotecting or having selectively deprotected the N-terminus of the first protected variant of Peptide B from step (A) (iii) (a) to form a second protected variant of Peptide B with a free amino group at the N-terminus;
(B) coupling or having coupled a protected variant of Fragment 1 with a protected variant of Fragment 2B to form a protected variant of Peptide D, wherein the protected variant of Fragment 1 has a free carboxyl group at the C-terminus and a protected N-terminus, the protected variant of Fragment 2B has a free amino group at the N-terminus, Fragment 1 comprises the amino acid sequence of SEQ ID NO: 1, Fragment 2B comprises the amino acid sequence of SEQ ID NO: 16, and Peptide D comprises the amino acid sequence of SEQ ID NO: 15; and
(C) coupling or having coupled the second protected variant of Peptide B from step (A) with the protected variant of Peptide D from step (B) to form a protected variant of Peptide A, wherein Peptide A comprises the amino acid sequence of SEQ ID NO: 7.

Provided herein as Embodiment B.34 is a method of preparing Peptide A or a protected variant thereof, comprising:
(A)
(i) (a) coupling or having coupled a protected variant of Fragment 3 with a protected variant of Fragment 4 to form a first protected variant of Peptide E, wherein the protected variant of Fragment 3 has a free carboxyl group at the C-terminus and a protected N-terminus, the protected variant of Fragment 4 has a free amino group at the N-terminus, Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8; (b) selectively deprotecting or having selectively deprotected the N-terminus of the first protected variant of Peptide E from step (A) (i) (a) to form a second protected variant of Peptide E with a free amino group at the N-terminus; (c) coupling or having coupled the second protected variant of Peptide E from step (A) (i) (b) with an N-protected alanine to form a first protected variant of Peptide B, wherein Peptide B comprises the amino acid sequence of SEQ ID NO: 9; and (d) selectively deprotecting or having selectively deprotected the N-terminus of the first protected variant of Peptide B from step (A) (i) (c) to form a second protected variant of Peptide B with a free amino group at the N-terminus; OR
(ii) (a) coupling or having coupled a protected variant of Fragment 3A with a protected variant of Fragment 4 to form a first protected variant of Peptide B, wherein the protected variant of Fragment 3A has a free carboxyl group at the C-terminus and a protected N-terminus, the protected variant of Fragment 4 has a free amino group at the N-terminus, Fragment 3A comprises the amino acid sequence of SEQ ID NO: 13, Fragment 4 comprises the amino acid sequence of SEQ ID NO: 4, and Peptide B comprises the amino acid sequence of SEQ ID NO: 9; and (b) selectively deprotecting or having selectively deprotected the N-terminus of the first protected variant of Peptide B from step (A) (ii) (a) to form a second protected variant of Peptide B with a free amino group at the N-terminus; OR
(iii) (a) coupling or having coupled a protected variant of Fragment 5 with a protected variant of Fragment 6 to form a first protected variant of Peptide B, wherein the protected variant of Fragment 5 has a free carboxyl group at the C-terminus and a protected N-terminus, the protected variant of Fragment 6 has a free amino group at the N-terminus, Fragment 5 comprises the amino acid sequence of SEQ ID NO: 5, Fragment 6 comprises the amino acid sequence of SEQ ID NO: 6, and Peptide B comprises the amino acid sequence of SEQ ID NO: 9; and (b) selectively deprotecting or having selectively deprotected the N-terminus of the first protected variant of Peptide B from step (A) (iii) (a) to form a second protected variant of Peptide B with a free amino group at the N-terminus;
(B) (i) coupling or having coupled a protected variant of Fragment 2A with the second protected variant of Peptide B from step (A) to form a first protected variant of Peptide C, wherein the protected variant of Fragment 2A has a free carboxyl group at the C-terminus and a protected N-terminus, Fragment 2A comprises the amino acid sequence of SEQ ID NO: 12, and Peptide C comprises the amino acid sequence of SEQ ID NO: 10; and (ii) selectively deprotecting or having selectively deprotected the N-terminus of the first protected variant of Peptide C from step (B) (i) to form a second protected variant of Peptide C with a free amino group at the N-terminus; and
(C) coupling or having coupled a protected variant of Fragment 1A with the second protected variant of Peptide C from step (B) (ii) to form a protected variant of Peptide A, wherein the protected variant of Fragment 1A has a free carboxyl group at the C-terminus and a protected N-terminus, Fragment 1A comprises the amino acid sequence of SEQ ID NO: 11, and Peptide A comprises the amino acid sequence of SEQ ID NO: 7.

Provided herein as Embodiment B.35 is a method of preparing Peptide A or a protected variant thereof, comprising:
coupling or having coupled a protected variant of Fragment 3 with a protected variant of Fragment 4 to form a first protected variant of Peptide E, wherein the protected variant of Fragment 3 has a free carboxyl group at the C-terminus and a protected N-terminus, the protected variant of Fragment 4 has a free amino group at the N-terminus, Fragment 3 comprises the amino acid sequence of SEQ ID NO: 3, Fragment 4 comprises the amino acid sequence of SEQ ID NO: 4, and Peptide E comprises the amino acid sequence of SEQ ID NO: 8;
selectively deprotecting or having selectively deprotected the N-terminus of the first protected variant of Peptide E to form a second protected variant of Peptide E with a free amino group at the N-terminus;
coupling or having coupled a protected variant of Fragment 2 with the second protected variant of Peptide E to form a first protected variant of Peptide F, wherein the protected variant of Fragment 2 has a free carboxyl group at the C-terminus and a protected N-terminus, Fragment 2 comprises the amino acid sequence of SEQ ID NO: 2, and Peptide F comprises the amino acid sequence of SEQ ID NO: 14;
selectively deprotecting or having selectively deprotected the N-terminus of the first protected variant of Peptide F to form a second protected variant of Peptide F with a free amino group at the N-terminus; and
coupling or having coupled a protected variant of Fragment 1 with the second protected variant of Peptide F to form a protected variant of Peptide A, wherein the protected variant of Fragment 1 has a free carboxyl group at the C-terminus and a protected N-terminus, Fragment 1 comprises the amino acid sequence of SEQ ID NO: 1, and Peptide A comprises the amino acid sequence of SEQ ID NO: 7.

Provided herein as Embodiment B.36 is a method of preparing Peptide A or a protected variant thereof, comprising:
coupling or having coupled a protected variant of Fragment 3A with a protected variant of Fragment 4 to form a first protected variant of Peptide B, wherein the protected variant of Fragment 3A has a free carboxyl group at the C-terminus and a protected N-terminus, the protected variant of Fragment 4 has a free amino group at the N-terminus, Fragment 3A comprises the amino acid sequence of SEQ ID NO: 3, Fragment 4 comprises the amino acid sequence of SEQ ID NO: 4, and Peptide B comprises the amino acid sequence of SEQ ID NO: 9;
selectively deprotecting or having selectively deprotected the N-terminus of the first protected variant of Peptide B to form a second protected variant of Peptide B with a free amino group at the N-terminus;
coupling or having coupled a protected variant of Fragment 2A with the second protected variant of Peptide B to form a first protected variant of Peptide C, wherein the protected variant of Fragment 2A has a free carboxyl group at the C-terminus and a protected (preferably Fmoc-protected) N-terminus, Fragment 2A comprises the amino acid sequence of SEQ ID NO: 12, and Peptide C comprises the amino acid sequence of SEQ ID NO: 10;
selectively deprotecting or having selectively deprotected the N-terminus of the first protected variant of Peptide C to form a second protected variant of Peptide C with a free amino group at the N-terminus; and

coupling or having coupled a protected variant of Fragment 1A with the second protected variant of Peptide C to form a protected variant of Peptide A, wherein the protected variant of Fragment 1A has a free carboxyl group at the C-terminus and a protected (preferably Boc-protected) N-terminus, Fragment 1A comprises the amino acid sequence of SEQ ID NO: 11, and Peptide A comprises the amino acid sequence of SEQ ID NO: 7.

Provided herein as Embodiment B.37 is the method of any one of Embodiments B.33 to B.36, wherein the method further comprises deprotecting or having deprotected the protected variant of Peptide A to isolate Peptide A.

Provided herein as Embodiment B.38 is the method of Embodiment B.37, wherein the method further comprises adding or having added a bromoacetyl moiety to the Lys at the C-terminus of Peptide A or a protected variant thereof.

Provided herein as Embodiment B.39 is a molecule selected from:

(SEQ ID NO: 18)
His(Trt)-Aib-Glu(OtBu)-Gly-Thr(tBu)-Phe-Thr(tBu)-
Ser(tBu)-Asp(Ompe)-Tyr(tBu)-Ser(tBu)-
Ser(Psi(Me,Me)Pro)-OH;
(SEQ ID NO: 19)
His(Boc)-Aib-Glu(OtBu)-Gly-Thr(tBu)-Phe-Thr(tBu)-
Ser(tBu)-Asp(Ompe)-Tyr(tBu)-Ser(tBu)-
Ser(Psi(Me,Me)Pro)-OH;
(SEQ ID NO: 20)
His(Boc)-Aib-Glu(OtBu)-Gly-Thr(tBu)-Phe-Thr(tBu)-
Ser(tBu)-Asp(OtBu)-Tyr(tBu)-Ser(tBu)-Ser(tBu)-
Tyr(tBu)-OH;
(SEQ ID NO: 21)
His(Trt)-Aib-Glu(OtBu)-Gly-Thr(tBu)-Phe-Thr(tBu)-
Ser(tBu)-Asp(OtBu)-Tyr(tBu)-Ser(tBu)-Ser(tBu)-
Tyr(tBu)-OH;
and
(SEQ ID NO: 22)
His(Boc)-Aib-Glu(OtBu)-Gly-Thr(tBu)-Phe-Thr(tBu)-
Ser(tBu)-Asp(OtBu)-Tyr(tBu)-Ser(tBu)-
Ser(Psi(Me,Me)Pro)-Tyr(tBu)-Leu-Glu(OtBu)-
Glu(OtBu)-Gln(Trt)-Ala-Ala-Lys(Boc)-Glu(OtBu)-
Phe-Ile-OH,

or a salt thereof.

Provided herein as Embodiment B.40 is a molecule selected from:

(SEQ ID NO: 23)
His(Trt)-Aib-Glu(OtBu)-Gly-Thr(tBu)-Phe-Thr(tBu)-
Ser(tBu)-Asp(OMpe)-Tyr(tBu)-Ser(tBu)-
Ser(Psi(Me,Me)Pro)-OH;
(SEQ ID NO: 24)
His(Boc)-Aib-Glu(OtBu)-Gly-Thr(tBu)-Phe-Thr(tBu)-
Ser(tBu)-Asp(OMpe)-Tyr(tBu)-Ser(tBu)-
Ser(Psi(Me,Me)Pro)-OH;
(SEQ ID NO: 25)
His(Boc)-Aib-Glu(OtBu)-Gly-Thr(tBu)-Phe-Thr(tBu)-
Ser(tBu)-Asp(OtBu)-Tyr(tBu)-Ser(tBu)-Ser(tBu)-
Tyr(tBu)-OH;
(SEQ ID NO: 26)
His(Trt)-Aib-Glu(OtBu)-Gly-Thr(tBu)-Phe-Thr(tBu)-
Ser(tBu)-Asp(OtBu)-Tyr(tBu)-Ser(tBu)-Ser(tBu)-
Tyr(tBu)-OH;
and
(SEQ ID NO: 27)
His(Boc)-Aib-Glu(OtBu)-Gly-Thr(tBu)-Phe-Thr(tBu)-
Ser(tBu)-Asp(OtBu)-Tyr(tBu)-Ser(tBu)-
Ser(Psi(Me,Me)Pro)-Tyr(tBu)-Leu-Glu(OtBu)-
Glu(OtBu)-Gln(Trt)-Ala-Ala-Lys(Boc)-Glu(OtBu)-
Phe-Ile-OH,

wherein the molecule has a protected N-terminus, or a salt thereof.

Provided herein as Embodiment B.41 is the molecule of Embodiment B.40, wherein the molecule has a Boc-protected N-terminus, or a salt thereof.

Provided herein as Embodiment B.42 is a molecule selected from:

(SEQ ID NO: 28)
Tyr(tBu)-Leu-Glu(OtBu)-Glu(OtBu)-Gln(Trt)-Ala-
Ala-Lys(Boc)-Glu(OtBu)-Phe-Ile-Ala-OH;
(SEQ ID NO: 29)
Leu-Glu(OtBu)-Glu(OtBu)-Gln(Trt)-Ala-Ala-
Lys(Boc)-Glu(OtBu)-Phe-Ile-OH;
(SEQ ID NO: 30)
Tyr(tBu)-Leu-Glu(OtBu)-Glu(OtBu)-Gln(Trt)-Ala-
Ala-Lys(Boc)-Glu(OtBu)-Phe-Ile-OH;
(SEQ ID NO: 31)
Leu-Glu(OtBu)-Glu(OtBu)-Gln(Trt)-Ala-Ala-
Lys(Boc)-Glu(OtBu)-Phe-Ile-Ala-OH;
(SEQ ID NO: 32)
Trp(Boc)-Leu-Val-Lys(Boc)-Gly-Gly-Gly-Gly-Gly-
Gly-Gly-OH;
(SEQ ID NO: 33)
Ala-Trp(Boc)-Leu-Val-Lys(Boc)-Gly-Gly-Gly-Gly-
Gly-Gly-Gly-OH;
(SEQ ID NO: 34)
Ala-Trp(Boc)-Leu-Val-Lys(Boc)-Gly-Gly-Gly-OH;
and
(SEQ ID NO: 35)
Ala-Trp(Boc)-Leu-Val-Lys-Gly-Gly-Gly-OH,

or a salt thereof.

Provided herein as Embodiment B.43 is a molecule selected from: Tyr(tBu)-Leu-Glu(OtBu)-Glu(OtBu)-Gln(Trt)-Ala-Ala-Lys(Boc)-Glu(OtBu)-Phe-Ile-Ala-OH (SEQ ID NO: 36);

(SEQ ID NO: 37)
Leu-Glu(OtBu)-Glu(OtBu)-Gln(Trt)-Ala-Ala-
Lys(Boc)-Glu(OtBu)-Phe-Ile-OH;
(SEQ ID NO: 38)
Tyr(tBu)-Leu-Glu(OtBu)-Glu(OtBu)-Gln(Trt)-Ala-
Ala-Lys(Boc)-Glu(OtBu)-Phe-Ile-OH;
(SEQ ID NO: 39)
Leu-Glu(OtBu)-Glu(OtBu)-Gln(Trt)-Ala-Ala-
Lys(Boc)-Glu(OtBu)-Phe-Ile-Ala-OH;
(SEQ ID NO: 40)
Trp(Boc)-Leu-Val-Lys(Boc)-Gly-Gly-Gly-Gly-Gly-
Gly-Gly-OH;
(SEQ ID NO: 41)
Ala-Trp(Boc)-Leu-Val-Lys(Boc)-Gly-Gly-Gly-Gly-
Gly-Gly-Gly-OH;
(SEQ ID NO: 42)
Ala-Trp(Boc)-Leu-Val-Lys(Boc)-Gly-Gly-Gly-OH;
and
(SEQ ID NO: 43)
Ala-Trp(Boc)-Leu-Val-Lys-Gly-Gly-Gly-OH,

wherein the molecule has a protected N-terminus, or a salt thereof.

Provided herein as Embodiment B.44 is a molecule selected from:

(SEQ ID NO: 44)
Tyr(tBu)-Leu-Glu(OtBu)-Glu(OtBu)-Gln(Trt)-Ala-
Ala-Lys(Boc)-Glu(OtBu)-Phe-Ile-Ala;
(SEQ ID NO: 45)
Leu-Glu(OtBu)-Glu(OtBu)-Gln(Trt)-Ala-Ala-
Lys(Boc)-Glu(OtBu)-Phe-Ile;
(SEQ ID NO: 46)
Tyr(tBu)-Leu-Glu(OtBu)-Glu(OtBu)-Gln(Trt)-Ala-
Ala-Lys(Boc)-Glu(OtBu)-Phe-Ile;
(SEQ ID NO: 47)
Leu-Glu(OtBu)-Glu(OtBu)-Gln(Trt)-Ala-Ala-
Lys(Boc)-Glu(OtBu)-Phe-Ile-Ala;
(SEQ ID NO: 48)
Trp(Boc)-Leu-Val-Lys(Boc)-Gly-Gly-Gly-Gly-Gly-
Gly-Gly;
(SEQ ID NO: 49)
Ala-Trp(Boc)-Leu-Val-Lys(Boc)-Gly-Gly-Gly-Gly-
Gly-Gly-Gly;
(SEQ ID NO: 50)
Ala-Trp(Boc)-Leu-Val-Lys(Boc)-Gly-Gly-Gly;
and
(SEQ ID NO: 51)
Ala-Trp(Boc)-Leu-Val-Lys-Gly-Gly-Gly,

wherein the molecule has a protected C-terminus, or a salt thereof.

Provided herein as Embodiment B.45 is the molecule of Embodiment B.43, wherein the molecule is selected from:

(SEQ ID NO: 52)
Fmoc-Tyr(tBu)-Leu-Glu(OtBu)-Glu(OtBu)-Gln(Trt)-
Ala-Ala-Lys(Boc)-Glu(OtBu)-Phe-Ile-Ala-OH;
(SEQ ID NO: 53)
Fmoc-Leu-Glu(OtBu)-Glu(OtBu)-Gln(Trt)-Ala-Ala-
Lys(Boc)-Glu(OtBu)-Phe-Ile-OH;
(SEQ ID NO: 54)
Fmoc-Tyr(tBu)-Leu-Glu(OtBu)-Glu(OtBu)-Gln(Trt)-
Ala-Ala-Lys(Boc)-Glu(OtBu)-Phe-Ile-OH;
(SEQ ID NO: 55)
Fmoc-Leu-Glu(OtBu)-Glu(OtBu)-Gln(Trt)-Ala-Ala-
Lys(Boc)-Glu(OtBu)-Phe-Ile-Ala-OH;
(SEQ ID NO: 56)
Fmoc-Trp(Boc)-Leu-Val-Lys(Boc)-Gly-Gly-Gly-Gly-
Gly-Gly-Gly-OH;
(SEQ ID NO: 57)
Fmoc-Ala-Trp(Boc)-Leu-Val-Lys(Boc)-Gly-Gly-Gly-
Gly-Gly-Gly-Gly-OH;
(SEQ ID NO: 58)
Fmoc-Ala-Trp(Boc)-Leu-Val-Lys(Boc)-Gly-Gly-Gly-OH;
(SEQ ID NO: 59)
Trt-Ala-Trp(Boc)-Leu-Val-Lys(Boc)-Gly-Gly-Gly-OH;
and
(SEQ ID NO: 60)
Fmoc-Ala-Trp(Boc)-Leu-Val-Lys-Gly-Gly-Gly-OH,

or a salt thereof.

Provided herein as Embodiment B.46 is a molecule selected from:

(SEQ ID NO: 61)
Ser(tBu)-Gly-Gly-Gly-Gly-Ser(tBu)-Gly-Gly-Gly-
Gly-Ser(tBu)-Lys(ivDde);
(SEQ ID NO: 62)
Gly-Gly-Gly-Gly-Ser(Psi(Me,Me)Pro)-Gly-Gly-
Gly-Gly-Ser(Psi(Me,Me)Pro)-Gly-Gly-Gly-Gly-
Ser(Psi(Me,Me)Pro)-Lys(ivDde);
(SEQ ID NO: 63)
Gly-Gly-Gly-Gly-Ser(tBu)-Gly-Gly-Gly-Gly-
Ser(tBu)-Gly-Gly-Gly-Gly-Ser(tBu)-Lys(ivDde);
(SEQ ID NO: 64)
Ala-Trp(Boc)-Leu-Val-Lys(Boc)-Gly-Gly-Gly-Gly-
Gly-Gly-Gly-Ser(tBu)-Gly-Gly-Gly-Gly-Ser(tBu)-
Gly-Gly-Gly-Gly-Ser(tBu)-Lys(ivDde);
(SEQ ID NO: 65)
Leu-Glu(OtBu)-Glu(OtBu)-Gln(Trt)-Ala-Ala-
Lys(Boc)-Glu(OtBu)-Phe-Ile-Ala-Trp(Boc)-Leu-
Val-Lys(Boc)-Gly-Gly-Gly-Gly-Gly-Gly-Gly-
Ser(tBu)-Gly-Gly-Gly-Gly-Ser(tBu)-Gly-Gly-
Gly-Gly-Ser(tBu)-Lys(ivDde);
(SEQ ID NO: 66)
Trp(Boc)-Leu-Val-Lys(Boc)-Gly-Gly-Gly-Gly-Gly-
Gly-Gly-Ser(tBu)-Gly-Gly-Gly-Gly-Ser(tBu)-Gly-
Gly-Gly-Gly-Ser(tBu)-Lys(ivDde);
and
(SEQ ID NO: 67)
Tyr(tBu)-Leu-Glu(OtBu)-Glu(OtBu)-Gln(Trt)-Ala-
Ala-Lys(Boc)-Glu(OtBu)-Phe-Ile-Ala-Trp(Boc)-
Leu-Val-Lys(Boc)-Gly-Gly-Gly-Gly-Gly-Gly-Gly-
Ser(tBu)-Gly-Gly-Gly-Gly-Ser(tBu)-Gly-Gly-Gly-
Gly-Ser(tBu)-Lys(ivDde),

wherein the molecule does not have a free C-terminal carboxyl group, or a salt thereof.

Provided herein as Embodiment B.47 is the molecule of Embodiment B.46, wherein the molecule has a protected C-terminus.

Provided herein as Embodiment B.48 is the molecule of Embodiment B.46, wherein the molecule has an amidated C-terminus.

Provided herein as Embodiment B.49 is a molecule selected from:

(SEQ ID NO: 68)
Ser(tBu)-Gly-Gly-Gly-Gly-Ser(tBu)-Gly-Gly-Gly-
Gly-Ser(tBu)-Lys(ivDde);
(SEQ ID NO: 69)
Gly-Gly-Gly-Gly-Ser(Psi(Me,Me)Pro)-Gly-Gly-Gly-
Gly-Ser(Psi(Me,Me)Pro)-Gly-Gly-Gly-Gly-Ser
(Psi(Me,Me)Pro)-Lys(ivDde);
(SEQ ID NO: 70)
Gly-Gly-Gly-Gly-Ser(tBu)-Gly-Gly-Gly-Gly-
Ser(tBu)-Gly-Gly-Gly-Gly-Ser(tBu)-Lys(ivDde);
(SEQ ID NO: 71)
Ala-Trp(Boc)-Leu-Val-Lys(Boc)-Gly-Gly-Gly-Gly-
Gly-Gly-Gly-Ser(tBu)-Gly-Gly-Gly-Gly-Ser(tBu)-
Gly-Gly-Gly-Gly-Ser(tBu)-Lys(ivDde);
(SEQ ID NO: 72)
Ala-Trp(Boc)-Leu-Val-Lys(Boc)-Gly-Gly-Gly-Gly-
Gly-Gly-Gly-Ser(Psi(Me,Me)Pro)-Gly-Gly-Gly-
Gly-Ser(Psi(Me,Me)Pro)-Gly-Gly-Gly-Gly-Ser
(Psi(Me,Me)Pro)-Lys(ivDde);
(SEQ ID NO: 73)
Leu-Glu(OtBu)-Glu(OtBu)-Gln(Trt)-Ala-Ala-
Lys(Boc)-Glu(OtBu)-Phe-Ile-Ala-Trp(Boc)-Leu-
Val-Lys(Boc)-Gly-Gly-Gly-Gly-Gly-Gly-Gly-
Ser(tBu)-Gly-Gly-Gly-Gly-Ser(tBu)-Gly-Gly-
Gly-Gly-Ser(tBu)-Lys(ivDde);
(SEQ ID NO: 74)
Trp(Boc)-Leu-Val-Lys(Boc)-Gly-Gly-Gly-Gly-
Gly-Gly-Gly-Ser(tBu)-Gly-Gly-Gly-Gly-
Ser(tBu)-Gly-Gly-Gly-Gly-Ser(tBu)-Lys(ivDde);
and
(SEQ ID NO: 75)
Tyr(tBu)-Leu-Glu(OtBu)-Glu(OtBu)-Gln(Trt)-
Ala-Ala-Lys(Boc)-Glu(OtBu)-Phe-Ile-Ala-
Trp(Boc)-Leu-Val-Lys(Boc)-Gly-Gly-Gly-Gly-
Gly-Gly-Gly-Ser(tBu)-Gly-Gly-Gly-Gly-
Ser(tBu)-Gly-Gly-Gly-Gly-Ser(tBu)-Lys(ivDde),

wherein the molecule has a protected N-terminus and does not have a free C-terminal carboxyl group, or a salt thereof.

Provided herein as Embodiment B.50 is the molecule of Embodiment B.49, wherein the molecule is selected from:

(SEQ ID NO: 76)
Fmoc-Ser(tBu)-Gly-Gly-Gly-Gly-Ser(tBu)-Gly-Gly-
Gly-Gly-Ser(tBu)-Lys(ivDde);
(SEQ ID NO: 77)
Fmoc-Gly-Gly-Gly-Gly-Ser(tBu)-Gly-Gly-Gly-Gly-
Ser(tBu)-Gly-Gly-Gly-Gly-Ser(tBu)-Lys(ivDde);
(SEQ ID NO: 78)
Trt-Gly-Gly-Gly-Gly-Ser(Psi(Me,Me)Pro)-Gly-Gly-
Gly-Gly-Ser(Psi(Me,Me)Pro)-Gly-Gly-Gly-Gly-
Ser(Psi(Me,Me)Pro)-Lys(ivDde);
(SEQ ID NO: 79)
Fmoc-Ala-Trp(Boc)-Leu-Val-Lys(Boc)-Gly-Gly-Gly-
Gly-Gly-Gly-Gly-Ser(tBu)-Gly-Gly-Gly-Gly-
Ser(tBu)-Gly-Gly-Gly-Gly-Ser(tBu)-Lys(ivDde);
(SEQ ID NO: 80)
Fmoc-Ala-Trp(Boc)-Leu-Val-Lys(Boc)-Gly-Gly-Gly-
Gly-Gly-Gly-Gly-Ser(Psi(Me,Me)Pro)-Gly-Gly-Gly-
Gly-Ser(Psi(Me,Me)Pro)-Gly-Gly-Gly-Gly-
Ser(Psi(Me,Me)Pro)-Lys(ivDde);
(SEQ ID NO: 81)
Fmoc-Leu-Glu(OtBu)-Glu(OtBu)-Gln(Trt)-Ala-Ala-
Lys(Boc)-Glu(OtBu)-Phe-Ile-Ala-Trp(Boc)-Leu-Val-
Lys(Boc)-Gly-Gly-Gly-Gly-Gly-Gly-Gly-Ser(tBu)-
Gly-Gly-Gly-Gly-Ser(tBu)-Gly-Gly-Gly-Gly-
Ser(tBu)-Lys(ivDde);
(SEQ ID NO: 82)
Fmoc-Trp(Boc)-Leu-Val-Lys(Boc)-Gly-Gly-Gly-Gly-
Gly-Gly-Gly-Ser(tBu)-Gly-Gly-Gly-Gly-Ser(tBu)-
Gly-Gly-Gly-Gly-Ser(tBu)-Lys(ivDde);
and
(SEQ ID NO: 83)
Fmoc-Tyr(tBu)-Leu-Glu(OtBu)-Glu(OtBu)-Gln(Trt)-
Ala-Ala-Lys(Boc)-Glu(OtBu)-Phe-Ile-Ala-Trp(Boc)-
Leu-Val-Lys(Boc)-Gly-Gly-Gly-Gly-Gly-Gly-Gly-
Ser(tBu)-Gly-Gly-Gly-Gly-Ser(tBu)-Gly-Gly-Gly-
Gly-Ser(tBu)-Lys(ivDde),

further wherein the molecule does not have a free C-terminal carboxyl group, or a salt thereof.

Provided herein as Embodiment B.51 is the molecule of Embodiment B.50, wherein the molecule has a protected C-terminus.

Provided herein as Embodiment B.52 is the molecule of Embodiment B.50, wherein the molecule has an amidated C-terminus.

Provided herein as Embodiment C.1 is a method of preparing Peptide B or a protected variant thereof, the method comprising coupling a protected variant of Fragment 5 with a protected variant of Fragment 6 in solution to form a protected variant of Peptide B, wherein Peptide B has the amino acid sequence of SEQ ID NO: 9, Fragment 5 has the amino acid sequence of SEQ ID NO: 5, and Fragment 6 has the amino acid sequence of SEQ ID NO: 6, further wherein:
the N-terminus, the side chain of the Trp residue at position 2, and the side chain of the Lys residue at position 5 of the protected variant of Fragment 5 are protected and the protected variant of Fragment 5 has a free carboxyl group at the C-terminus; and
the side chain of the Ser residue at position 5, the side chain of the Ser residue at position 10, the side chain of the Ser residue at position 15, and the side chain of the Lys residue at position 16 of the protected variant of Fragment 6 are protected, the protected variant of Fragment 6 has a free amino group at the N-terminus, and the protected variant of Fragment 6 does not have a free carboxyl group at the C-terminus.

Provided herein as Embodiment C.2 is the method of Embodiment C.1, wherein the coupling is mediated by a coupling reagent and optionally a base and/or a coupling additive.

Provided herein as Embodiment C.3 is the method of Embodiment C.1 or Embodiment C.2, wherein the coupling is mediated by 1 to 10 equivalents of a coupling reagent, optionally 1 to 10 equivalents of a base, and optionally 1 to 10 equivalents of a coupling additive, wherein equivalents are relative to the protected variant of Fragment 5.

Provided herein as Embodiment C.4 is the method of any one of Embodiments C.1 to C.3, wherein the coupling is mediated by 1 to 5 equivalents of a coupling reagent, optionally 1 to 10 equivalents of a base, and optionally 1 to 5 equivalents of a coupling additive, wherein equivalents are relative to the protected variant of Fragment 5.

Provided herein as Embodiment C.5 is the method of any one of Embodiments C.2 to C.4, wherein the coupling reagent is selected from N,N′-diisopropylcarbodiimide (DIC), N,N′-dicyclohexylcarbodiimide (DCC), 1-ethyl-3-(3-dimethylaminopropyl) carbodiimide (EDC), O-(1H-benzotriazol-1-yl)-N,N,N′,N′-tetramethyluronium hexafluorophosphate (HBTU), O-(1H-benzotriazol-1-yl)-N,N,N′,N′-tetramethyluronium tetrafluoroborate (TBTU), O-(7-azabenzotriazol-1-yl)-N,N, N′,N′-tetramethyluronium hexafluorophosphate (HATU), O-(6-chlorobenzotriazol-1-yl)-N,N,N′,N′-tetramethyluronium hexafluorophosphate (HCTU), O-(7-azabenzotriazol-1-yl)-N,N,N′,N′-tetramethyluronium tetrafluoroborate (TATU), O-(6-chlorobenzotriazol-1-yl)-N,N,N′,N′-tetramethyluronium tetrafluoroborate (TCTU), 2-(1H-benzotriazol-1-yl)-1,1,3,3-tetramethylaminium nitrate (TNTU), N,N,N′,N′-tetramethyl-O—(N-succinimidyl) uronium tetrafluoroborate (TSTU), 1-[(1-(cyano-2-ethoxy-2-oxoethylideneaminooxy)dimethylaminomorpholino)]uronium hexafluorophosphate (COMU), O-[(ethoxycarbonyl) cyanomethylenamino]-N,N,N′,N′-tetramethyluronium tetrafluoroborate (TOTU), (benzotriazol-1-yloxy)tripyrrolidinophosphonium hexafluorophosphate (PyBOP), (7-azabenzotriazol-1-yloxy)tripyrrolidinophosphonium hexafluorophosphate (PyAOP), bromotripyrrolidinophosphonium hexafluorophosphate (PyBroP), chlorotripyrrolidinophosphonium hexafluorophosphate (PyCIOP), (benzotriazol-1-yloxy)tris(dimethylamino)phosphonium hexafluorophosphate (BOP), bis(2-oxo-3-oxazolidinyl)phosphinic chloride (BOP—Cl), (ethyl cyano(hydroxyimino)acetato-O²)tri(1-pyrrolidinyl)phosphonium hexafluorophosphate (PyOxim), propanephosphonic acid anhydride (T3P), 3-(diethoxyphosphoryloxy)-1,2,3-benzotriazin-4 (3H)-one (DEPBT), and combinations of any of the foregoing.

Provided herein as Embodiment C.6 is the method of any one of Embodiments C.2 to C.5, wherein the coupling reagent is selected from EDC, DIC, T3P, HATU, HBTU, HCTU, TBTU, PyBOP, PyOxim, and combinations of any of the foregoing.

Provided herein as Embodiment C.7 is the method of any one of Embodiments C.2 to C.6, wherein the coupling reagent is TBTU, PyBOP, or a combination thereof.

Provided herein as Embodiment C.8 is the method of any one of Embodiments C.2 to C.7, wherein the coupling reagent is PyBOP.

Provided herein as Embodiment C.9 is the method of any one of Embodiments C.2 to C.8, wherein the base is selected from N,N-diisopropylethylamine (DIPEA), N-methylmorpholine (NMM), N-ethylmaleimide (NEM), triethylamine (TEA), 2,6-lutidine, 2,4,6-collidine, 2,2,6,6-tetramethylpiperidine (TMP), and combinations of any of the foregoing.

Provided herein as Embodiment C. 10 is the method of any one of Embodiments C.2 to C.9, wherein the base is selected from DIPEA, TEA, 2,6-lutidine, 2,4,6-collidine and combinations of any of the foregoing.

Provided herein as Embodiment C.11 is the method of any one of Embodiments C.2 to C.10, wherein the base is TEA, 2,4,6-collidine, or a combination thereof.

Provided herein as Embodiment C. 12 is the method of any one of Embodiments C.2 to C.11, wherein the base is TEA.

Provided herein as Embodiment C. 13 is the method of any one of Embodiments C.2 to C. 12, wherein the coupling additive is selected from 4-(dimethylamino)pyridine (DMAP), 1-hydroxybenzotriazole (HOBt), 1-hydroxy-6-chlorobenzotriazole (6-Cl—HOBt), 1-hydroxy-7-azabenzotriazole (HOAt), 2-pyridinol 1-oxide (HOPO), ethyl 2-cyano-2-(hydroxyimino)acetate (Oxyma), and combinations of any of the foregoing.

Provided herein as Embodiment C. 14 is the method of any one of Embodiments C.1 to C.4, wherein the coupling is mediated by DIC/Oxyma, DIC/HOPO, EDC/DMAP, EDC/HOBt/2,4,6-collidine, HATU/2,4,6-collidine, HBTU/2,4,6-collidine, HCTU/2,4,6-collidine, PyBOP/2,4,6-collidine, PyBOP/TEA, PyOxim/Oxyma, PyOxim/Oxyma/2,4,6-collidine, T3P/Oxyma, T3P/Oxyma/2,4,6-collidine, or TBTU/2,4,6-collidine.

Provided herein as Embodiment C. 15 is the method of any one of Embodiments C.1 to C. 12 or C.14, wherein the coupling is mediated by PyBOP and TEA.

Provided herein as Embodiment C. 16 is the method of any one of Embodiments C.1 to C.12, C.14, or C.15, wherein the coupling is mediated by 1 to 10 equivalents of PyBOP and 1 to 10 equivalents of TEA, wherein equivalents are relative to the protected variant of Fragment 5.

Provided herein as Embodiment C. 17 is the method of any one of Embodiments C.1 to C.12 or C.14 to C.16, wherein the coupling is mediated by 2 to 5 equivalents of PyBOP and 2 to 10 equivalents of TEA, wherein equivalents are relative to the protected variant of Fragment 5.

Provided herein as Embodiment C. 18 is the method of any one of Embodiments C.1 to C.17, wherein the molar ratio of the protected variant of Fragment 5 to the protected variant of Fragment 6 in solution prior to the coupling is in the range of 1:1 to 1:1.5.

Provided herein as Embodiment C. 19 is the method of any one of Embodiments C.1 to C. 18, wherein the molar ratio of the protected variant of Fragment 5 to the protected variant of Fragment 6 in solution prior to the coupling is in the range of 1:1 to 1:1.2.

Provided herein as Embodiment C.20 is the method of any one of Embodiments C.1 to C. 19, wherein the molar ratio of the protected variant of Fragment 5 to the protected variant of Fragment 6 in solution prior to the coupling is in the range of 1:1.05 to 1:1.15.

Provided herein as Embodiment C.21 is the method of any one of Embodiments C.1 to C.21, wherein the protected variant of Fragment 5 is the limiting reactant for the coupling.

Provided herein as Embodiment C.22 is the method of Embodiment C.1, wherein the coupling is mediated by 1 to 10 equivalents of PyBOP and 1 to 10 equivalents of TEA, wherein equivalents are relative to the protected variant of Fragment 5, and further wherein the molar ratio of the protected variant of Fragment 5 to the protected variant of Fragment 6 in solution prior to the coupling is in the range of 1:1 to 1:1.5.

Provided herein as Embodiment C.23 is the method of Embodiment C.1, wherein the coupling is mediated by 2 to 5 equivalents of PyBOP and 2 to 10 equivalents of TEA, wherein equivalents are relative to the protected variant of Fragment 5, and further wherein the molar ratio of the protected variant of Fragment 5 to the protected variant of Fragment 6 in solution prior to the coupling is in the range of 1:1 to 1:1.2.

Provided herein as Embodiment C.24 is the method of any one of Embodiments C.1 to C.23, wherein the coupling is conducted at a temperature in the range of 10° C. to 60° C.

Provided herein as Embodiment C.25 is the method of any one of Embodiments C.1 to C.24, wherein the coupling is conducted at a temperature in the range of 20° C. to 50° C.

Provided herein as Embodiment C.26 is the method of any one of Embodiments C.1 to C.25, wherein the coupling is conducted at a temperature in the range of 20° C. to 40° C.

Provided herein as Embodiment C.27 is the method of any one of Embodiments C.1 to C.26, wherein the coupling is conducted at a temperature in the range of 25° C. to 35° C.

Provided herein as Embodiment C.28 is the method of any one of Embodiments C.1 to C.27, wherein the coupling is conducted for a time in the range of 1 hour to 72 hours.

Provided herein as Embodiment C.29 is the method of any one of Embodiments C.1 to C.28, wherein the coupling is conducted for a time in the range of 1 hour to 6 hours.

Provided herein as Embodiment C.30 is the method of any one of Embodiments C.1 to C.29, wherein the coupling is conducted for a time in the range of 1 hour to 2 hours.

Provided herein as Embodiment C.31 is the method of any one of Embodiments C.1 to C.23, wherein the coupling is conducted at a temperature in the range of 20° C. to 40° C. for a time in the range of 1 hour to 72 hours.

Provided herein as Embodiment C.32 is the method of any one of Embodiments C.1 to C.23, wherein the coupling is conducted at a temperature in the range of 25° C. to 35° C. for a time in the range of 1 hour to 2 hours.

Provided herein as Embodiment C.33 is the method of any one of Embodiments C.1 to C.32, wherein the coupling is conducted in a solvent system comprising an organic solvent.

Provided herein as Embodiment C.34 is the method of any one of Embodiments C.1 to C.33, wherein the coupling is conducted in dimethyl sulfoxide (DMSO) and methanol (MeOH).

Provided herein as Embodiment C.35 is the method of any one of Embodiments C.1 to C.34, wherein the coupling is conducted in 1-5:1 DMSO:MeOH.

Provided herein as Embodiment C.36 is the method of any one of Embodiments C.1 to C.35, wherein the coupling is conducted in 1-3:1 DMSO:MeOH.

Provided herein as Embodiment C.37 is the method of any one of Embodiments C.1 to C.36, wherein the coupling is conducted in 1:1 DMSO:MeOH.

Provided herein as Embodiment C.38 is the method of any one of Embodiments C.1 to C.26, wherein the coupling is conducted in 1-5:1 DMSO:MeOH at a temperature in the range of 20° C. to 40° C. for a time in the range of 1 hour to 72 hours.

Provided herein as Embodiment C.39 is the method of any one of Embodiments C.1 to C.27, wherein the coupling is conducted in 1-3:1 DMSO:MeOH at a temperature in the range of 25° C. to 35° C. for a time in the range of 1 hour to 2 hours.

Provided herein as Embodiment C.40 is the method of Embodiment C.1, wherein the coupling is conducted in 1-5:1 DMSO:MeOH at a temperature in the range of 20° C. to 40° C. for a time in the range of 1 hour to 72 hours, wherein the coupling is mediated by 1 to 10 equivalents of PyBOP and 1 to 10 equivalents of TEA, wherein equivalents are relative to the protected variant of Fragment 5, and further wherein the molar ratio of the protected variant of Fragment 5 to the protected variant of Fragment 6 in solution prior to the coupling is in the range of 1:1 to 1:1.5.

Provided herein as Embodiment C.41 is the method of Embodiment C.1, wherein the coupling is conducted in 1-3:1 DMSO:MeOH at a temperature in the range of 25° C. to 35° C. for a time in the range of 1 hour to 2 hours, wherein the coupling is mediated by 2 to 5 equivalents of PyBOP and 2 to 10 equivalents of TEA, wherein equivalents are relative to the protected variant of Fragment 5, and further wherein the molar ratio of the protected variant of Fragment 5 to the protected variant of Fragment 6 in solution prior to the coupling is in the range of 1:1 to 1:1.2.

Provided herein as Embodiment C.42 is the method of any one of Embodiments C.1 to C.41, wherein:
the side chain of the Trp residue at position 2 and the side chain of the Lys residue at position 5 of the protected variant of Fragment 5 are each protected with acid-labile protecting groups;
the side chain of the Ser residue at position 5, the side chain of the Ser residue at position 10, and the side chain of the Ser residue at position 15 of the protected variant of Fragment 6 are each protected with acid-labile protecting groups; and
the side chain of the Lys residue at position 16 of the protected variant of Fragment 6 is orthogonally protected relative to the side chain protecting groups of the protected variant of Fragment 5 and the protected variant of Fragment 6 and the N-terminal protecting group of the protected variant of Fragment 5.

Provided herein as Embodiment C.43 is the method of any one of Embodiments C.1 to C.42, wherein:
the side chain of the Trp residue at position 2 and the side chain of the Lys residue at position 5 of the protected variant of Fragment 5 are each protected with a tert-butyloxycarbonyl (Boc) protecting group; and
the Ser residues at position 5, position 10, and position 15 of the protected variant of Fragment 6 are each protected as a dimethylated pseudoproline (Psi (Me,Me) pro) moiety in which the oxazolidine is derived from Ser and the side chain of the Lys residue at position 16 of the protected variant of Fragment 6 is protected with an ivDde (1-(4,4-dimethyl-2,6-dioxocyclohex-1-ylidene)-3-methylbutyl) protecting group.

Provided herein as Embodiment C.44 is the method of any one of Embodiments C.1 to C.43, wherein the N-terminus of the protected variant of Fragment 5 is protected with a trityl (Trt) protecting group.

Provided herein as Embodiment C.45 is the method of any one of Embodiments C.1 to C.44, wherein the C-terminus of the protected variant of Fragment 6 is protected.

Provided herein as Embodiment C.46 is the method of any one of Embodiments C.1 to C.44, wherein the C-terminus of the protected variant of Fragment 6 is amidated as a C-terminal primary amide.

Provided herein as Embodiment C.47 is the method of any one of Embodiments C.1 to C.46, wherein the protected variant of Fragment 5 is Trt-Ala-Trp(Boc)-Leu-Val-Lys(Boc)-Gly-Gly-Gly-OH (SEQ ID NO: 59).

Provided herein as Embodiment C.48 is the method of any one of Embodiments C.1 to C.44, C.46, or C.47, wherein the protected variant of Fragment 6 is H-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130).

Provided herein as Embodiment C.49 is the method of any one of Embodiments C.1 to C.44 or C.46 to C.48, wherein the protected variant of Fragment 5 is Trt-Ala-Trp(Boc)-Leu-Val-Lys(Boc)-Gly-Gly-Gly-OH (SEQ ID NO: 59) and the protected variant of Fragment 6 is H-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130).

Provided herein as Embodiment C.50 is the method of any one of Embodiments C.1 to C.49, wherein the method further comprises deprotecting the N-terminus of the protected variant of Peptide B formed by the coupling to form a second protected variant of Peptide B with a free amino group at the N-terminus.

Provided herein as Embodiment C.51 is the method of Embodiment C.50, wherein the N-terminus of the protected variant of Peptide B is protected with a trityl (Trt) protecting group and the deprotecting comprises contacting the protected variant of Peptide B with 1 to 10 equivalents of a Brønsted acid or a Lewis acid, wherein equivalents are relative to the protected variant of Peptide B.

Provided herein as Embodiment C.52 is the method of Embodiment C.50 or Embodiment C.51, wherein the N-terminus of the protected variant of Peptide B is protected with a Trt protecting group and the deprotecting comprises contacting the protected variant of Peptide B with 1 to 10 equivalents of trifluoroacetic acid (TFA) or hydrochloric acid (HCl), wherein equivalents are relative to the protected variant of Peptide B.

Provided herein as Embodiment C.53 is the method of any one of Embodiments C.50 to C.52, wherein the N-terminus of the protected variant of Peptide B is protected with a Trt protecting group and the deprotecting comprises contacting the protected variant of Peptide B with 2 to 5 equivalents of HCl, wherein equivalents are relative to the protected variant of Peptide B.

Provided herein as Embodiment C.54 is the method of any one of Embodiments C.50 to C.53, wherein the method further comprises isolating the second protected variant of Peptide B by antisolvent precipitation.

Provided herein as Embodiment C.55 is the method of Embodiment C.54, wherein the antisolvent is ethanol.

Provided herein as Embodiment C.56 is the method of Embodiment C.54 or Embodiment C.55, wherein the isolated second protected variant of Peptide B is slurry washed with ethanol and then displacement washed with acetonitrile.

Provided herein as Embodiment C.57 is the method of any one of Embodiments C.1 to C.50, wherein the method further comprises deprotecting the N-terminus of the protected variant of Peptide B formed by the coupling to form a second protected variant of Peptide B with a free amino group at the N-terminus, isolating the second protected variant of Peptide B by antisolvent precipitation with ethanol, and slurry washing the isolated second protected variant of Peptide B with ethanol followed by displacement washing with acetonitrile.

Provided herein as Embodiment C.58 is the method of any one of Embodiments C.1 to C.57, wherein the protected variant of Fragment 5 and/or the protected variant of Fragment 6 is provided in the form of a salt.

Provided herein as Embodiment C.59 is a method of preparing a Peptide D or a protected variant thereof, the method comprising coupling a protected variant of Fragment 1 with a protected variant of Fragment 2B in solution to form a protected variant of Peptide D, wherein Peptide D has the amino acid sequence of SEQ ID NO: 15, Fragment 1 has the amino acid sequence of SEQ ID NO: 1, and Fragment 2B has the amino acid sequence of SEQ ID NO: 16, further wherein:
the N-terminus, the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, and the side chain of the Ser residue at position 12 of the protected variant of Fragment 1 are protected and the protected variant of Fragment 1 has a free carboxyl group at the C-terminus; and
the side chain of the Tyr residue at position 1, the side chain of the Glu residue at position 3, the side chain of the Glu residue at position 4, the side chain of the Gln residue at position 5, the side chain of the Lys residue at position 8, and the side chain of the Glu residue at position 9 of the protected variant of Fragment 2B are protected and the protected variant of Fragment 2B has a free amino group at the N-terminus.

Provided herein as Embodiment C.60 is the method of Embodiment C.59, wherein the coupling is mediated by a coupling reagent and optionally a base and/or a coupling additive.

Provided herein as Embodiment C.61 is the method of Embodiment C.59 or Embodiment C.60, wherein the coupling is mediated by 1 to 10 equivalents of a coupling reagent, optionally 1 to 10 equivalents of a base, and optionally 1 to 10 equivalents of a coupling additive, wherein equivalents are relative to the protected variant of Fragment 2B.

Provided herein as Embodiment C.62 is the method of any one of Embodiments C.59 to C.61, wherein the coupling is mediated by 1 to 5 equivalents of a coupling reagent, optionally 1 to 10 equivalents of a base, and optionally 1 to 5 equivalents of a coupling additive, wherein equivalents are relative to the protected variant of Fragment 2B.

Provided herein as Embodiment C.63 is the method of any one of Embodiments C.60 to C.62, wherein the coupling reagent is selected from N,N′-diisopropylcarbodiimide (DIC), N,N′-dicyclohexylcarbodiimide (DCC), 1-ethyl-3-(3-dimethylaminopropyl) carbodiimide (EDC), O-(1H-benzotriazol-1-yl)-N,N,N′,N′-tetramethyluronium hexafluorophosphate (HBTU), O-(1H-benzotriazol-1-yl)-N,N,N′,N′-tetramethyluronium tetrafluoroborate (TBTU), O-(7-azabenzotriazol-1-yl)-N,N,N′,N′-tetramethyluronium hexafluorophosphate (HATU), O-(6-chlorobenzotriazol-1-yl)-N,N,N′,N′-tetramethyluronium hexafluorophosphate (HCTU), O-(7-azabenzotriazol-1-yl)-N,N,N′,N′-tetramethyluronium tetrafluoroborate (TATU), O-(6-chlorobenzotriazol-1-yl)-N,N,N′,N′-tetramethyluronium tetrafluoroborate (TCTU), 2-(1H-benzotriazol-1-yl)-1,1,3,3-tetramethylaminium nitrate (TNTU), N,N,N′,N′-tetramethyl-O—(N-succinimidyl) uronium tetrafluoroborate (TSTU), 1-[(1-(cyano-2-ethoxy-2-oxoethylideneaminooxy)dimethylaminomorpholino)]uronium hexafluorophosphate (COMU), O-[(ethoxycarbonyl) cyanomethylenamino]-N,N,N′,N′-tetramethyluronium tetrafluoroborate (TOTU), (benzotriazol-1-yloxy)tripyrrolidinophosphonium hexafluorophosphate (PyBOP), (7-azabenzotriazol-1-yloxy)tripyrrolidinophosphonium hexafluorophosphate (PyAOP), bromotripyrrolidinophosphonium hexafluorophosphate (PyBroP), chlorotripyrrolidinophosphonium hexafluorophosphate (PyCIOP), (benzotriazol-1-yloxy)tris(dimethylamino)phosphonium hexafluorophosphate (BOP), bis(2-oxo-3-oxazolidinyl)phosphinic chloride (BOP—Cl), (ethyl cyano(hydroxyimino)acetato-O2)tri(1-pyrrolidinyl)phosphonium hexafluorophosphate (PyOxim), propanephosphonic acid anhydride (T3P), 3-(diethoxyphosphoryloxy)-1,2,3-benzotriazin-4 (3H)-one (DEPBT), and combinations of any of the foregoing.

Provided herein as Embodiment C.64 is the method of any one of Embodiments C.60 to C.63, wherein the coupling reagent is selected from EDC, DIC, T3P, HATU, HBTU, HCTU, TBTU, PyBOP, PyOxim, and combinations of any of the foregoing.

Provided herein as Embodiment C.65 is the method of any one of Embodiments C.60 to C.64, wherein the coupling reagent is PyBOP.

Provided herein as Embodiment C.66 is the method of any one of Embodiments C.60 to C.65, wherein the base is selected from N,N-diisopropylethylamine (DIPEA), N-methylmorpholine (NMM), N-ethylmaleimide (NEM), triethylamine (TEA), 2,6-lutidine, 2,4,6-collidine, 2,2,6,6-tetramethylpiperidine (TMP), and combinations of any of the foregoing.

Provided herein as Embodiment C.67 is the method of any one of Embodiments C.60 to C.66, wherein the base is 2,4,6-collidine.

Provided herein as Embodiment C.68 is the method of any one of Embodiments C.60 to C.67, wherein the coupling additive is selected from 4-(dimethylamino)pyridine (DMAP), 1-hydroxybenzotriazole (HOBt), 1-hydroxy-6-chlorobenzotriazole (6-Cl—HOBt), 1-hydroxy-7-azabenzotriazole (HOAt), 2-pyridinol 1-oxide (HOPO), ethyl 2-cyano-2-(hydroxyimino)acetate (Oxyma), and combinations of any of the foregoing.

Provided herein as Embodiment C.69 is the method of any one of Embodiments C.59 to C.62, wherein the coupling is mediated by DIC/Oxyma, DIC/HOPO, EDC/DMAP, EDC/HOBt/2,4,6-collidine, HATU/2,4,6-collidine, HBTU/2,4,6-collidine, HCTU/2,4,6-collidine, PyBOP/2,4,6-collidine, PyBOP/TEA, PyOxim/Oxyma, PyOxim/Oxyma/2,4,6-collidine, T3P/Oxyma, T3P/Oxyma/2,4,6-collidine, or TBTU/2,4,6-collidine.

Provided herein as Embodiment C.70 is the method of any one of Embodiments C.59 to C.62, wherein the coupling is mediated by PyBOP and 2,4,6-collidine.

Provided herein as Embodiment C.71 is the method of Embodiment C.59, wherein the coupling is mediated by 1 to 10 equivalents of PyBOP and 1 to 10 equivalents of 2,4,6-collidine, wherein equivalents are relative to the protected variant of Fragment 2B.

Provided herein as Embodiment C.72 is the method of Embodiment C.59, wherein the coupling is mediated by 1 to 5 equivalents of PyBOP and 2 to 10 equivalents of 2,4,6-collidine, wherein equivalents are relative to the protected variant of Fragment 2B.

Provided herein as Embodiment C.73 is the method of any one of Embodiments C.59 to C.72, wherein the molar ratio of the protected variant of Fragment 2B to the protected variant of Fragment 1 in solution prior to the coupling is in the range of 1:1 to 1:2.

Provided herein as Embodiment C.74 is the method of any one of Embodiments C.59 to C.73, wherein the molar ratio of the protected variant of Fragment 2B to the protected variant of Fragment 1 in solution prior to the coupling is in the range of 1:1 to 1:1.5.

Provided herein as Embodiment C.75 is the method of any one of Embodiments C.59 to C.74, wherein the molar ratio of the protected variant of Fragment 2B to the protected variant of Fragment 1 in solution prior to the coupling is in the range of 1:1 to 1:1.2.

Provided herein as Embodiment C.76 is the method of any one of Embodiments C.59 to C.75, wherein the protected variant of Fragment 2B is the limiting reactant for the coupling.

Provided herein as Embodiment C.77 is the method of Embodiment C.59, wherein the coupling is mediated by 1 to 10 equivalents of PyBOP and 1 to 10 equivalents of 2,4,6-collidine, wherein equivalents are relative to the protected variant of Fragment 2B, and further wherein the molar ratio of the protected variant of Fragment 2B to the protected variant of Fragment 1 in solution prior to the coupling is in the range of 1:1 to 1:1.5.

Provided herein as Embodiment C.78 is the method of Embodiment C.59, wherein the coupling is mediated by 1 to 5 equivalents of PyBOP and 1 to 10 equivalents of 2,4,6-collidine, wherein equivalents are relative to the protected variant of Fragment 2B, and further wherein the molar ratio of the protected variant of Fragment 2B to the protected variant of Fragment 1 in solution prior to the coupling is in the range of 1:1 to 1:1.2.

Provided herein as Embodiment C.79 is the method of any one of Embodiments C.59 to C.78, wherein the coupling is conducted at a temperature in the range of 10° C. to 60° C.

Provided herein as Embodiment C.80 is the method of any one of Embodiments C.59 to C.79, wherein the coupling is conducted at a temperature in the range of 20° C. to 50° C.

Provided herein as Embodiment C.81 is the method of any one of Embodiments C.59 to C.80, wherein the coupling is conducted at a temperature in the range of 20° C. to 40° C.

Provided herein as Embodiment C.82 is the method of any one of Embodiments C.59 to C.81, wherein the coupling is conducted for a time in the range of 1 hour to 72 hours.

Provided herein as Embodiment C.83 is the method of any one of Embodiments C.59 to C.82, wherein the coupling is conducted for a time in the range of 1 hour to 6 hours.

Provided herein as Embodiment C.84 is the method of any one of Embodiments C.59 to C.83, wherein the coupling is conducted for a time in the range of 1 hour to 2 hours.

Provided herein as Embodiment C.85 is the method of any one of Embodiments C.59 to C.80, wherein the coupling is conducted at a temperature in the range of 20° C. to 50° C. for a time in the range of 1 hour to 72 hours.

Provided herein as Embodiment C.86 is the method of any one of Embodiments C.59 to C.81, wherein the coupling is conducted at a temperature in the range of 20° C. to 40° C. for a time in the range of 1 hour to 2 hours.

Provided herein as Embodiment C.87 is the method of any one of Embodiments C.59 to C.86, wherein the coupling is conducted in a solvent system comprising an organic solvent.

Provided herein as Embodiment C.88 is the method of any one of Embodiments C.59 to C.87, wherein the coupling is conducted in tetrahydrofuran (THF) and dimethyl sulfoxide (DMSO).

Provided herein as Embodiment C.89 is the method of any one of Embodiments C.59 to C.88, wherein the coupling is conducted in 5-8:1 THF:DMSO.

Provided herein as Embodiment C.90 is the method of any one of Embodiments C.59 to C.89, wherein the coupling is conducted in 7:1 THF:DMSO.

Provided herein as Embodiment C.91 is the method of any one of Embodiments C.59 to C.80, wherein the coupling is conducted in 5-8:1 THF:DMSO at a temperature in the range of 20° C. to 50° C. for a time in the range of 1 hour to 72 hours.

Provided herein as Embodiment C.92 is the method of any one of Embodiments C.59 to C.81, wherein the coupling is conducted in 5-8:1 THF:DMSO at a temperature in the range of 20° C. to 40° C. for a time in the range of 1 hour to 2 hours.

Provided herein as Embodiment C.93 is the method of Embodiment C.59, wherein the coupling is conducted in 5-8:1 THF:DMSO at a temperature in the range of 20° C. to 50° C. for a time in the range of 1 hour to 72 hours, wherein the coupling is mediated by 1 to 10 equivalents of PyBOP and 1 to 10 equivalents of 2,4,6-collidine, wherein equivalents are relative to the protected variant of Fragment 2B, and further wherein the molar ratio of the protected variant of Fragment 2B to the protected variant of Fragment 1 in solution prior to the coupling is in the range of 1:1 to 1:1.5.

Provided herein as Embodiment C.94 is the method of Embodiment C.59, wherein the coupling is conducted in 5-8:1 THF:DMSO at a temperature in the range of 20° C. to 40° C. for a time in the range of 1 hour to 2 hours, wherein the coupling is mediated by 1 to 5 equivalents of PyBOP and 1 to 5 equivalents of 2,4,6-collidine, wherein equivalents are relative to the protected variant of Fragment 2B, and further wherein the molar ratio of the protected variant of Fragment 2B to the protected variant of Fragment 1 in solution prior to the coupling is in the range of 1:1 to 1:1.2.

Provided herein as Embodiment C.95 is the method of any one of Embodiments C.59 to C.94, wherein:
the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, and the side chain of the Ser residue at position 12 of the protected variant of Fragment 1 are each independently protected with an acid-labile protecting group; and
the side chain of the Tyr residue at position 1, the side chain of the Glu residue at position 3, the side chain of the Glu residue at position 4, the side chain of the Gln residue at position 5, the side chain of the Lys residue at position 8, and the side chain of the Glu residue at position 9 of the protected variant of Fragment 2B are each independently protected with an acid-labile protecting group.

Provided herein as Embodiment C.96 is the method of any one of Embodiments C.59 to C.95, wherein the side chain of the His residue at position 1 of the protected variant of Fragment 1 is protected with a trityl (Trt) protecting group; the side chain of the Glu residue at position 3 of the protected variant of Fragment 1 is protected with a tert-butyl ester (OtBu) protecting group; the side chains of the Thr residues at position 5 and position 7 of the protected variant of Fragment 1 are each protected with a tert-butyl (tBu) protecting group; the side chains of the Ser residues at position 8 and position 11 of the protected variant of Fragment 1 are each protected with a tBu protecting group; the side chain of the Asp residue at position 9 of the protected variant of Fragment 1 is protected with an OtBu protecting group; the side chain of the Tyr residue at position 10 of the protected variant of Fragment 1 is protected with a tBu protecting group; and the Ser residue at position 12 of the protected variant of Fragment 1 is protected as a dimethylated pseudoproline (Psi (Me,Me) pro) moiety in which the oxazolidine is derived from Ser.

Provided herein as Embodiment C.97 is the method of any one of Embodiments C.59 to C.96, wherein the side chain of the Tyr residue at position 1 of the protected variant of Fragment 2B is protected with a tBu protecting group; the side chains of the Glu residues at position 3, position 4, and position 9 of the protected variant of Fragment 2B are each protected with a OtBu protecting group; the side chain of the Gln residue at position 5 of the protected variant of Fragment 2B is protected with a Trt protecting group; and the side chain of the Lys residue at position 8 of the protected variant of Fragment 2B is protected with a tert-butyloxycarbonyl (Boc) protecting group

Provided herein as Embodiment C.98 is the method of any one of Embodiments C.59 to C.97, wherein:
the side chain of the His residue at position 1 of the protected variant of Fragment 1 is protected with a trityl (Trt) protecting group; the side chain of the Glu residue at position 3 of the protected variant of Fragment 1 is protected with a tert-butyl ester (OtBu) protecting group; the side chains of the Thr residues at position 5 and position 7 of the protected variant of Fragment 1 are each protected with a tert-butyl (tBu) protecting group; the side chains of the Ser residues at position 8 and position 11 of the protected variant of Fragment 1 are each protected with a tBu protecting group; the side chain of the Asp residue at position 9 of the protected variant of Fragment 1 is protected with an OtBu protecting group; the side chain of the Tyr residue at position 10 of the protected variant of Fragment 1 is protected with a tBu protecting group; and the Ser residue at position 12 of the protected variant of Fragment 1 is protected as a dimethylated pseudoproline (Psi (Me, Me) pro) moiety in which the oxazolidine is derived from Ser; and
the side chain of the Tyr residue at position 1 of the protected variant of Fragment 2B is protected with a tBu protecting group; the side chains of the Glu residues at position 3, position 4, and position 9 of the protected variant of Fragment 2B are each protected with a OtBu protecting group; the side chain of the Gln residue at position 5 of the protected variant of Fragment 2B is protected with a Trt protecting group; and the side chain of the Lys residue at position 8 of the protected variant of Fragment 2B is protected with a tert-butyloxycarbonyl (Boc) protecting group

Provided herein as Embodiment C.99 is the method of any one of Embodiments C.59 to C.98, wherein the protected variant of Fragment 2B has a free carboxyl group at the C-terminus.

Provided herein as Embodiment C. 100 is the method of any one of Embodiments C.59 to C.99, wherein the protected variant of Fragment 2B has a free carboxyl group at the C-terminus and the C-terminal carboxyl group of the protected variant of Fragment 1 is pre-activated.

Provided herein as Embodiment C. 101 is the method of any one of Embodiments C.59 to C.98, wherein the C-terminus of the protected variant of Fragment 2B is protected.

Provided herein as Embodiment C. 102 is the method of any one of Embodiments C.59 to C.101, wherein the protected variant of Fragment 1 is Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 166)

Provided herein as Embodiment C. 103 is the method of any one of Embodiments C.59 to C.100 or C.102, wherein the protected variant of Fragment 2B is H-Tyr(tBu)-Leu-Glu(OtBu)-Glu(OtBu)-Gln(Trt)-Ala-Ala-Lys(Boc)-Glu(OtBu)-Phe-Ile-OH.

Provided herein as Embodiment C. 104 is the method of any one of Embodiments C.59 to C.100, C.102, or C.103, wherein the protected variant of Fragment 1 is Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 166) and the protected variant of Fragment 2B is H-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 30).

Provided herein as Embodiment C. 105 is the method of any one of Embodiments C.59 to C.104, wherein the method further comprises isolating the protected variant of Peptide D formed by the coupling by antisolvent precipitation.

Provided herein as Embodiment C. 106 is the method of Embodiment C. 105, wherein the antisolvent is water.

Provided herein as Embodiment C. 107 is the method of any one of Embodiments C.59 to C.98, C. 101, or C.102, wherein the C-terminus of the protected variant of Fragment 2B is protected, and the method further comprises deprotecting the C-terminus of the protected variant of Peptide D formed by the coupling to form a second protected variant of Peptide D with a free carboxyl group at the C-terminus.

Provided herein as Embodiment C. 108 is the method of Embodiment C. 107, wherein the method further comprises isolating the second protected variant of Peptide D by antisolvent precipitation.

Provided herein as Embodiment C. 109 is the method of Embodiment C. 108, wherein the antisolvent is water.

Provided herein as Embodiment C. 110 is the method of any one of Embodiments C.59 to C.109, wherein the protected variant of Fragment 1 and/or the protected variant of Fragment 2B is provided in the form of a salt.

Provided herein as Embodiment C. 111 is a method of preparing Peptide A or a variant thereof, the method comprising coupling a protected variant of Peptide B with a protected variant of Peptide D to form a protected variant of Peptide A, wherein Peptide A has the amino acid sequence of SEQ ID NO: 7, Peptide B has the amino acid sequence of SEQ ID NO: 9, and Peptide D has the amino acid sequence of SEQ ID NO: 15, further wherein:
the side chain of the Trp residue at position 2, the side chain of the Lys residue at position 5, the side chain of the Ser residue at position 13, the side chain of the Ser residue at position 18, the side chain of the Ser residue at position 23, and the side chain of the Lys residue at position 24 of the protected variant of Peptide B are protected, the protected variant of Peptide B has a free amino group at the N-terminus, and the protected variant of Peptide B does not have a free carboxyl group at the C-terminus; and
the N-terminus, the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, the side chain of the Ser residue at position 12, the side chain of the Tyr residue at position 13, the side chain of the Glu residue at position 15, the side chain of the Glu residue at position 16, the side chain of the Gln residue at position 17, the side chain of the Lys residue at position 20, and the side chain of the Glu residue at position 21 of the protected variant of Peptide D are protected and the protected variant of Peptide D has a free carboxyl group at the C-terminus.

Provided herein as Embodiment C. 112 is the method of Embodiment C.111, wherein the coupling is mediated by a coupling reagent and optionally a base and/or a coupling additive.

Provided herein as Embodiment C.113 is the method of Embodiment C.111 or Embodiment C.112, wherein the coupling is mediated by 1 to 10 equivalents of a coupling reagent, optionally 1 to 10 equivalents of a base, and optionally 1 to 10 equivalents of a coupling additive, wherein equivalents are relative to the protected variant of Peptide D.

Provided herein as Embodiment C.114 is the method of any one of Embodiments C.111 to C.113, wherein the coupling is mediated by 1 to 5 equivalents of a coupling reagent, optionally 1 to 10 equivalents of a base, and optionally 1 to 5 equivalents of a coupling additive, wherein equivalents are relative to the protected variant of Peptide D.

Provided herein as Embodiment C.115 is the method of any one of Embodiments C.112 to C.114, wherein the coupling reagent is selected from N,N′-diisopropylcarbodiimide (DIC), N,N′-dicyclohexylcarbodiimide (DCC), 1-ethyl-3-(3-dimethylaminopropyl) carbodimide (EDC), O-(1H-benzotriazol-1-yl)-N,N,N′,N′-tetramethyluronium hexafluorophosphate (HBTU), O-(1H-benzotriazol-1-yl)-N,N,N′,N′-tetramethyluronium tetrafluoroborate (TBTU), O-(7-azabenzotriazol-1-yl)-N,N,N′,N′-tetramethyluronium hexafluorophosphate (HATU), O-(6-chlorobenzotriazol-1-yl)-N,N,N′,N′-tetramethyluronium hexafluorophosphate (HCTU), O-(7-azabenzotriazol-1-yl)-N,N,N′,N′-tetramethyluronium tetrafluoroborate (TATU), O-(6-chlorobenzotriazol-1-yl)-N,N,N′,N′-tetramethyluronium tetrafluoroborate (TCTU), 2-(1H-benzotriazol-1-yl)-1,1,3,3-tetramethylaminium nitrate (TNTU), N,N,N′,N′-tetramethyl-O—(N-succinimidyl) uronium tetrafluoroborate (TSTU), 1-[(1-(cyano-2-ethoxy-2-oxoethylideneaminooxy)dimethylaminomorpholino)]uronium hexafluorophosphate (COMU), O-[(ethoxycarbonyl) cyanomethylenamino]-N, N,N′,N′-tetramethyluronium tetrafluoroborate (TOTU), (benzotriazol-1-yloxy)tripyrrolidinophosphonium hexafluorophosphate (PyBOP), (7-azabenzotriazol-1-yloxy)tripyrrolidinophosphonium hexafluorophosphate (PyAOP), bromotripyrrolidinophosphonium hexafluorophosphate (PyBroP), chlorotripyrrolidinophosphonium hexafluorophosphate (PyCIOP), (benzotriazol-1-yloxy)tris(dimethylamino)phosphonium hexafluorophosphate (BOP), bis(2-oxo-3-oxazolidinyl)phosphinic chloride (BOP—Cl), (ethyl cyano(hydroxyimino)acetato-O2)tri(1-pyrrolidinyl)phosphonium hexafluorophosphate (PyOxim), propanephosphonic acid anhydride (T3P), 3-(diethoxyphosphoryloxy)-1,2,3-benzotriazin-4 (3H)-one (DEPBT), and combinations of any of the foregoing.

Provided herein as Embodiment C. 116 is the method of any one of Embodiments C.112 to C.115, wherein the coupling reagent is selected from EDC, DIC, T3P, HATU, HBTU, HCTU, TBTU, PyBOP, PyOxim, and combinations of any of the foregoing.

Provided herein as Embodiment C. 117 is the method of any one of Embodiments C.112 to C. 116, wherein the coupling reagent is EDC.

Provided herein as Embodiment C.118 is the method of any one of Embodiments C.112 to C. 117, wherein the base is selected from N,N-diisopropylethylamine (DIPEA), N-methylmorpholine (NMM), N-ethylmaleimide (NEM), triethylamine (TEA), 2,6-lutidine, 2,4,6-collidine, 2,2,6,6-tetramethylpiperidine (TMP), and combinations of any of the foregoing.

Provided herein as Embodiment C. 119 is the method of any one of Embodiments C.112 to C. 118, wherein the base is 2,4,6-collidine.

Provided herein as Embodiment C. 120 is the method of any one of Embodiments C.112 to C.119, wherein the coupling additive is selected from 4-(dimethylamino)pyridine (DMAP), 1-hydroxybenzotriazole (HOBt), 1-hydroxy-6-chlorobenzotriazole (6-Cl—HOBt), 1-hydroxy-7-azabenzotriazole (HOAt), 2-pyridinol 1-oxide (HOPO), ethyl 2-cyano-2-(hydroxyimino)acetate (Oxyma), and combinations of any of the foregoing.

Provided herein as Embodiment C. 121 is the method of any one of Embodiments C.112 to C. 120, wherein the coupling additive is HOBt.

Provided herein as Embodiment C. 122 is the method of any one of Embodiments C.111 to C.114, wherein the coupling is mediated by DIC/Oxyma, DIC/HOPO, EDC/DMAP, EDC/HOBt/2,4,6-collidine, HATU/2,4,6-collidine, HBTU/2,4,6-collidine, HCTU/2,4,6-collidine, PyBOP/2,4,6-collidine, PyBOP/TEA, PyOxim/Oxyma, PyOxim/Oxyma/2,4,6-collidine, T3P/Oxyma, T3P/Oxyma/2,4,6-collidine, or TBTU/2,4,6-collidine.

Provided herein as Embodiment C. 123 is the method of any one of Embodiments C.111 to C. 122, wherein the coupling is mediated by EDC, 2,4,6-collidine, and HOBt.

Provided herein as Embodiment C. 124 is the method of Embodiment C. 111, wherein the coupling is mediated by 1 to 10 equivalents of EDC, 1 to 10 equivalents of 2,4,6-collidine, and 1 to 10 equivalents for HOBt, wherein equivalents are relative to the protected variant of Peptide D.

Provided herein as Embodiment C. 125 is the method of Embodiment C.111, wherein the coupling is mediated by 1 to 5 equivalents of EDC, 2 to 10 equivalents of 2,4,6-collidine, and 1 to 5 equivalents of HOBt, wherein equivalents are relative to the protected variant of Peptide D.

Provided herein as Embodiment C. 126 is the method of any one of Embodiments C.111 to C. 125, wherein the molar ratio of the protected variant of Peptide D to the protected variant of Peptide B in solution prior to the coupling is in the range of 1:1 to 1:2.

Provided herein as Embodiment C. 127 is the method of any one of Embodiments C.111 to C.126, wherein the molar ratio of the protected variant of Peptide D to the protected variant of Peptide B in solution prior to the coupling is in the range of 1:1 to 1:1.5.

Provided herein as Embodiment C. 128 is the method of any one of Embodiments C.111 to C.127, wherein the molar ratio of the protected variant of Peptide D to the protected variant of Peptide B in solution prior to the coupling is in the range of 1:1 to 1:1.25.

Provided herein as Embodiment C. 129 is the method of any one of Embodiments C.111 to C.128, wherein the protected variant of Peptide D is the limiting reactant for the coupling.

Provided herein as Embodiment C. 130 is the method of Embodiment C.111, wherein the coupling is mediated by 1 to 10 equivalents of EDC, 1 to 10 equivalents of 2,4,6-collidine, and 1 to 10 equivalents for HOBt, wherein equivalents are relative to the protected variant of Peptide D, and further wherein the molar ratio of the protected variant of Peptide D to the protected variant of Peptide B in solution prior to the coupling is in the range of 1:1 to 1:1.5.

Provided herein as Embodiment C. 131 is the method of Embodiment C.111, wherein the coupling is mediated by 1 to 5 equivalents of EDC, 2 to 10 equivalents of 2,4,6-collidine, and 1 to 5 equivalents of HOBt, wherein equivalents are relative to the protected variant of Peptide D, and further wherein the molar ratio of the protected variant of Peptide D to the protected variant of Peptide B in solution prior to the coupling is in the range of 1:1 to 1:1.25.

Provided herein as Embodiment C. 132 is the method of any one of Embodiments C.111 to C.131, wherein the coupling is conducted at a temperature in the range of 10° C. to 60° C.

Provided herein as Embodiment C. 133 is the method of any one of Embodiments C.111 to C.132, wherein the coupling is conducted at a temperature in the range of 20° C. to 50° C.

Provided herein as Embodiment C. 134 is the method of any one of Embodiments C.111 to C.133, wherein the coupling is conducted at a temperature in the range of 20° C. to 40° C.

Provided herein as Embodiment C. 135 is the method of any one of Embodiments C.111 to C. 134, wherein the coupling is conducted at a temperature in the range of 20° C. to 30° C.

Provided herein as Embodiment C. 136 is the method of any one of Embodiments C.111 to C. 135, wherein the coupling is conducted for a time in the range of 1 hour to 72 hours.

Provided herein as Embodiment C. 137 is the method of any one of Embodiments C.111 to C. 136, wherein the coupling is conducted for a time in the range of 1 hour to 36 hours.

Provided herein as Embodiment C. 138 is the method of any one of Embodiments C.111 to C.137, wherein the coupling is conducted for a time in the range of 1 hour to 24 hours.

Provided herein as Embodiment C. 139 is the method of any one of Embodiments C.111 to C.133, wherein the coupling is conducted at a temperature in the range of 20° C. to 50° C. for a time in the range of 1 hour to 72 hours.

Provided herein as Embodiment C. 140 is the method of any one of Embodiments C.111 to C.134, wherein the coupling is conducted at a temperature in the range of 20° C. to 40° C. for a time in the range of 1 hour to 36 hours.

Provided herein as Embodiment C. 141 is the method of any one of Embodiments C.111 to C.135, wherein the coupling is conducted at a temperature in the range of 20° C. to 30° C. for a time in the range of 1 hour to 24 hours.

Provided herein as Embodiment C. 142 is the method of any one of Embodiments C.111 to C.141, wherein the coupling is conducted in a solvent system comprising an organic solvent and water.

Provided herein as Embodiment C. 143 is the method of any one of Embodiments C.111 to C. 142, wherein the coupling is conducted in tetrahydrofuran (THF) and water.

Provided herein as Embodiment C. 144 is the method of any one of Embodiments C.111 to C. 143, wherein the coupling is conducted in 3-5:1 THF:H₂O.

Provided herein as Embodiment C. 145 is the method of any one of Embodiments C.111 to C.144, wherein the coupling is conducted in 4:1 THF:H₂O.

Provided herein as Embodiment C. 146 is the method of any one of Embodiments C.111 to C.134, wherein the coupling is conducted in 3-5:1 THF:H₂O at a temperature in the range of 20° C. to 40° C. for a time in the range of 1 hour to 36 hours.

Provided herein as Embodiment C.14 is the method of any one of Embodiments C.111 to C.135, wherein the coupling is conducted in 3-5:1 THF:H₂O at a temperature in the range of 20° C. to 30° C. for a time in the range of 1 hour to 24 hours.

Provided herein as Embodiment C. 148 is the method of Embodiment C.111, wherein the coupling is conducted in 3-5:1 THF:H₂O at a temperature in the range of 20° C. to 40° C. for a time in the range of 1 hour to 36 hours, wherein the coupling is mediated by 1 to 10 equivalents of EDC, 1 to 10 equivalents of 2,4,6-collidine, and 1 to 10 equivalents for HOBt, wherein equivalents are relative to the protected variant of Peptide D, and further wherein the molar ratio of the protected variant of Peptide D to the protected variant of Peptide B in solution prior to the coupling is in the range of 1:1 to 1:1.5.

Provided herein as Embodiment C. 149 is the method of Embodiment C.111, wherein the coupling is conducted in 3-5:1 THF:H₂O at a temperature in the range of 20° C. to 30° C. for a time in the range of 1 hour to 24 hours, wherein the coupling is mediated by 1 to 5 equivalents of EDC, 2 to 10 equivalents of 2,4,6-collidine, and 1 to 5 equivalents of HOBt, wherein equivalents are relative to the protected variant of Peptide D, and further wherein the molar ratio of the protected variant of Peptide D to the protected variant of Peptide B in solution prior to the coupling is in the range of 1:1 to 1:1.25.

Provided herein as Embodiment C. 150 is the method of any one of Embodiments C.111 to C.149, wherein:
the side chain of the Trp residue at position 2, the side chain of the Lys residue at position 5, the side chain of the Ser residue at position 13, the side chain of the Ser residue at position 18, and the side chain of the Ser residue at position 23 of the protected variant of Peptide B are each independently protected with an acid-labile protecting group;
the N-terminus, the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, the side chain of the Ser residue at position 12, the side chain of the Tyr residue at position 13, the side chain of the Glu residue at position 15, the side chain of the Glu residue at position 16, the side chain of the Gln residue at position 17, the side chain of the Lys residue at position 20, and the side chain of the Glu residue at position 21 of the protected variant of Peptide D are each independently protected with an acid-labile protecting group; and
the side chain of the Lys residue at position 24 of the protected variant of Peptide B is orthogonally protected relative to the side chain protecting groups of the protected variant of Peptide B and the protected variant of Peptide D and the N-terminal protecting group of the protected variant of Peptide D.

Provided herein as Embodiment C. 151 is the method of any one of Embodiments C.111 to C. 150, wherein the side chain of the Trp residue at position 2 and the side chain of the Lys residue at position 5 of the protected variant of Peptide B are each protected with a tert-butyloxycarbonyl (Boc) protecting group, the Ser residues at position 13, position 18, and position 23 of the protected variant of Peptide B are each protected as a dimethylated pseudoproline (Psi (Me, Me) pro) moiety in which the oxazolidine is derived from Ser, and the side chain of the Lys residue at position 24 of the protected variant of Peptide B is protected with an ivDde (1-(4,4-dimethyl-2,6-dioxocyclohex-1-ylidene)-3-methylbutyl) protecting group.

Provided herein as Embodiment C. 152 is the method of any one of Embodiments C.111 to C.151, wherein the side chain of the His residue at position 1 of Peptide D is protected with a trityl (Trt) protecting group; the side chains of the Glu residues at position 3, position 15, position 16, and position 21 of Peptide D are each protected with a tert-butyl ester (OtBu) protecting group; the side chains of the Thr residues at position 5 and position 7 of Peptide D are each protected with a tert-butyl (tBu) protecting group; the side chains of the Ser residues at position 8 and position 11 of Peptide D are each protected with a tBu protecting group; the side chain of the Asp residue at position 9 of Peptide D is protected with an OtBu protecting group; the side chains of the Tyr residues at positions 10 and 13 of Peptide D are each protected with a tBu protecting group; the Ser residue at position 12 of Peptide D is protected as a dimethylated pseudoproline (Psi (Me,Me) pro) moiety in which the oxazolidine is derived from Ser; the side chain of the Gln residue at position 17 of Peptide D is protected with a Trt protecting group; and the side chain of the Lys residue at position 20 of Peptide D is protected with a tert-butyloxycarbonyl (Boc) protecting group.

Provided herein as Embodiment C. 153 is the method of any one of Embodiments C.111 to C. 152, wherein:
the side chain of the Trp residue at position 2 and the side chain of the Lys residue at position 5 of the protected variant of Peptide B are each protected with a tert-butyloxycarbonyl (Boc) protecting group, the Ser residues at position 13, position 18, and position 23 of the protected variant of Peptide B are each protected as a dimethylated pseudoproline (Psi (Me,Me) pro) moiety in which the oxazolidine is derived from Ser, and the side chain of the Lys residue at position 24 of the protected variant of Peptide B is protected with an ivDde (1-(4,4-dimethyl-2,6-dioxocyclohex-1-ylidene)-3-methylbutyl) protecting group; and
the side chain of the His residue at position 1 of Peptide D is protected with a trityl (Trt) protecting group; the side chains of the Glu residues at position 3, position 15, position 16, and position 21 of Peptide D are each protected with a tert-butyl ester (OtBu) protecting group; the side chains of the Thr residues at position 5 and position 7 of Peptide D are each protected with a tert-butyl (tBu) protecting group; the side chains of the Ser residues at position 8 and position 11 of Peptide D are each protected with a tBu protecting group; the side chain of the Asp residue at position 9 of Peptide D is protected with an OtBu protecting group; the side chains of the Tyr residues at positions 10 and 13 of Peptide D are each protected with a tBu protecting group; the Ser residue at position 12 of Peptide D is protected as a dimethylated pseudoproline (Psi (Me, Me) pro) moiety in which the oxazolidine is derived from Ser; the side chain of the Gln residue at position 17 of Peptide D is protected with a Trt protecting group; and the side chain of the Lys residue at position 20 of Peptide D is protected with a tert-butyloxycarbonyl (Boc) protecting group.

Provided herein as Embodiment C. 154 is the method of any one of Embodiments C.111 to C.153, wherein the N-terminus of the protected variant of Peptide D is protected with a tert-butyloxycarbonyl (Boc) protecting group.

Provided herein as Embodiment C. 155 is the method of any one of Embodiments C.111 to C. 154, wherein the C-terminus of the protected variant of Peptide B is amidated as a C-terminal primary amide.

Provided herein as Embodiment C. 156 is the method of any one of Embodiments C.111 to C.155, wherein the protected variant of Peptide B is H-Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 147).

Provided herein as Embodiment C. 157 is the method of any one of Embodiments C.111 to C. 156, wherein the protected variant of Peptide D is Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 169).

Provided herein as Embodiment C. 158 is the method of any one of Embodiments C.111 to C. 157, wherein the protected variant of Peptide B is H-Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 147) and the protected variant of Peptide D is Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 169).

Provided herein as Embodiment C. 159 is the method of any one of Embodiments C.111 to C.158, wherein the protected variant of Peptide B and/or the protected variant of Peptide D is provided in the form of a salt.

Provided herein as Embodiment C. 160 is the method of any one of Embodiments C.111 to C. 159, wherein the method further comprises selectively deprotecting the side chain of the C-terminal Lys residue of the protected variant of Peptide A formed by the coupling to form a partially deprotected variant of Peptide A in which the only protecting group removed from the protected variant of Peptide A is the side chain protecting group of the C-terminal Lys residue.

Provided herein as Embodiment C. 161 is the method of Embodiment C. 160, wherein the side chain of the C-terminal Lys residue of the protected variant of Peptide A formed by the coupling is protected with an ivDde (1-(4,4-dimethyl-2,6-dioxocyclohex-1-ylidene)-3-methylbutyl) protecting group, and the selectively deprotecting comprises contacting the protected variant of Peptide A with hydrazine or hydroxylamine.

Provided herein as Embodiment C. 162 is the method of Embodiment C. 161, wherein the selectively deprotecting comprises contacting the protected variant of Peptide A with hydroxylamine.

Provided herein as Embodiment C. 163 is the method of Embodiment C. 161 or Embodiment C.162, wherein the selectively deprotecting comprises contacting the protected variant of Peptide A with hydroxylamine for a time in the range of 20 hours to 72 hours.

Provided herein as Embodiment C. 164 is the method of any one of Embodiments C. 161 to C. 163, wherein the selectively deprotecting comprises contacting the protected variant of Peptide A with hydroxylamine at a temperature in the range of 20° C. to 30° C.

Provided herein as Embodiment C. 165 is the method of any one of Embodiments C.161 to C.164, wherein the selectively deprotecting comprises contacting the protected variant of Peptide A with hydroxylamine at a temperature in the range of 20° C. to 30° C.

Provided herein as Embodiment C. 166 is the method of any one of Embodiments C.161 to C. 165, further comprising isolating the partially deprotected variant of Peptide A.

Provided herein as Embodiment C. 167 is the method of any one of Embodiments C. 160 to C. 166, wherein the method further comprises coupling the partially deprotected variant of Peptide A with bromoacetic acid or bromoacetic anhydride to form a bromoacetylated protected variant of Peptide A.

Provided herein as Embodiment C. 168 is the method of Embodiment C. 167, wherein the molar ratio of the partially deprotected variant of Peptide A to bromoacetic acid or bromoacetic anhydride prior to the coupling is in the range of 1:1 to 1:20.

Provided herein as Embodiment C. 16 is the method of Embodiment C. 167 or Embodiment C. 168, wherein the molar ratio of the partially deprotected variant of Peptide A to bromoacetic acid or bromoacetic anhydride prior to the coupling is in the range of 1:5 to 1:15.

Provided herein as Embodiment C. 170 is the method of any one of Embodiments C. 160 to C. 169, wherein the method further comprises coupling the partially deprotected variant of Peptide A with bromoacetic anhydride to form a bromoacetylated protected variant of Peptide A.

Provided herein as Embodiment C.171 is the method of any one of Embodiments C.167 to C.170, wherein the coupling is mediated by a coupling reagent and optionally a coupling additive and/or a base.

Provided herein as Embodiment C. 172 is the method of any one of Embodiments C.167 to C.171, wherein the coupling is mediated by 1 to 20 equivalents of a coupling reagent, optionally 1 to 10 equivalents of a coupling additive, and optionally 1 to 10 equivalents of a base, wherein equivalents are relative to the partially deprotected variant of Peptide A.

Provided herein as Embodiment C. 173 is the method of any one of Embodiments C.167 to C.172, wherein the coupling is mediated by DIC and 2,6-lutidine.

Provided herein as Embodiment C. 174 is the method of any one of Embodiments C.167 to C.173, wherein the coupling is mediated by 1 to 20 equivalents of DIC and 1 to 10 equivalents of 2,6-lutidine, wherein equivalents are relative to the partially deprotected variant of Peptide A.

Provided herein as Embodiment C. 175 is the method of any one of Embodiments C.167 to C.174, wherein the method further comprises coupling the partially deprotected variant of Peptide A with bromoacetic anhydride to form a bromoacetylated protected variant of Peptide A, wherein the coupling is mediated by 1 to 20 equivalents of DIC and 1 to 10 equivalents of 2,6-lutidine, wherein equivalents are relative to the partially deprotected variant of Peptide A, and further wherein the molar ratio of the partially deprotected variant of Peptide A to bromoacetic acid or bromoacetic anhydride prior to the coupling is in the range of 1:5 to 1:15.

Provided herein as Embodiment C. 176 is the method of any one of Embodiments C.167 to C. 175, wherein the coupling is conducted at a temperature in the range of 20° C. to 30° C.

Provided herein as Embodiment C. 177 is the method of any one of Embodiments C.167 to C. 176, wherein the coupling is conducted for a time in the range of 1 hour to 12 hours.

Provided herein as Embodiment C. 178 is the method of any one of Embodiments C.167 to C. 177, wherein the coupling is conducted at a temperature in the range of 20° C. to 30° C. for a time in the range of 1 hour to 12 hours.

Provided herein as Embodiment C. 179 is the method of any one of Embodiments C.167 to C. 178, wherein the method further comprises isolating the bromoacetylated protected variant of Peptide A.

Provided herein as Embodiment C. 180 is the method of any one of Embodiments C.167 to C. 179, wherein the method further comprises contacting the bromoacetylated protected variant of Peptide A with a Brønsted acid or a Lewis acid, optionally in the presence of a scavenger, to form a bromoacetylated variant of Peptide A, wherein the bromoacetylated variant of Peptide A has the amino acid sequence of SEQ ID NO: 1, the N-terminus and the side chains of the amino acids at positions 1-46 of the bromoacetylated variant of Peptide A are not protected, the C-terminal Lys residue is bromoacetylated, and the C-terminus of the bromoacetylated variant of Peptide A is amidated as a C-terminal primary amide.

Provided herein as Embodiment C. 181 is the method of any one of Embodiments C.167 to C. 180, wherein the method further comprises contacting the bromoacetylated protected variant of Peptide A with trifluoroacetic acid (TFA), optionally in the presence of a scavenger, to form a bromoacetylated variant of Peptide A, wherein the bromoacetylated variant of Peptide A has the amino acid sequence of SEQ ID NO: 1, the N-terminus and the side chains of the amino acids at positions 1-46 of the bromoacetylated variant of Peptide A are not protected, the C-terminal Lys residue is bromoacetylated, and the C-terminus of the bromoacetylated variant of Peptide A is amidated as a C-terminal primary amide.

Provided herein as Embodiment C. 182 is the method of Embodiment C. 180 or Embodiment C.181, wherein the scavenger is triisopropylsilane.

Provided herein as Embodiment C. 183 is the method of any one of Embodiments C.180 to C. 182, wherein the contacting is conducted at a temperature in the range of −10° C. to 10° C.

Provided herein as Embodiment C. 184 is the method of any one of Embodiments C.180 to C. 183, wherein the contacting is conducted at a temperature in the range of −5° C. to 5° C.

Provided herein as Embodiment C. 185 is the method of any one of Embodiments C.180 to C. 184, wherein the contacting is conducted for a time in the range of 12 hours to 48 hours.

Provided herein as Embodiment C. 186 is the method of any one of Embodiments C.180 to C. 185, wherein the contacting is conducted for a time in the range of 20 hours to 48 hours.

Provided herein as Embodiment C. 187 is the method of any one of Embodiments C.180 to C.182, wherein the contacting is conducted at a temperature in the range of −10° C. to 10° C. for a time in the range of 12 hours to 48 hours.

Provided herein as Embodiment C. 188 is the method of any one of Embodiments C.180 to C. 182, wherein the contacting is conducted at a temperature in the range of −5° C. to 5° C. for a time in the range of 20 hours to 48 hours.

Provided herein as Embodiment C. 189 is the method of any one of Embodiments C.180 to C. 188, wherein the method further comprises isolating the bromoacetylated variant of Peptide A.

Provided herein as Embodiment C. 190 is a method of preparing Peptide A or a variant thereof, comprising:
(A) coupling a protected variant of Fragment 5 with a protected variant of Fragment 6 to form a protected variant of Peptide B; and/or
(B) coupling a protected variant of Fragment 1 with a protected variant of Fragment 2B to form a protected variant of Peptide D; and/or
(C) coupling a protected variant of Peptide B with a protected variant of Peptide D to form a protected variant of Peptide A, wherein Peptide A has the amino acid sequence of SEQ ID NO: 7, Peptide B has the amino acid sequence of SEQ ID NO: 9, Peptide D has the amino acid sequence of SEQ ID NO: 15, Fragment 1 has the amino acid sequence of SEQ ID NO: 1, Fragment 2B has the amino acid sequence of SEQ ID NO: 16, Fragment 5 has the amino acid sequence of SEQ ID NO: 5, and Fragment 6 has the amino acid sequence of SEQ ID NO: 6.

Provided herein as Embodiment C. 191 is the method of Embodiment C. 190, wherein the method comprises coupling a protected variant of Fragment 5 with a protected variant of Fragment 6 to form a protected variant of Peptide B according to the method of any one of Embodiments 1 to 58.

Provided herein as Embodiment C. 192 is the method of Embodiment C. 190 or Embodiment C. 191, wherein the method comprises coupling a protected variant of Fragment 1 with a protected variant of Fragment 2B to form a protected variant of Peptide D according to the method of any one of Embodiments 59 to 110.

Provided herein as Embodiment C. 193 is the method of any one of Embodiments C.190 to C. 192, wherein the method comprises coupling a protected variant of Peptide B with a protected variant of Peptide D to form a protected variant of Peptide A according to the method of any one of Embodiments 111 to 159.

Provided herein as Embodiment C. 194 is the method of Embodiment C. 190, wherein the coupling of any of step (A), step (B), or step (C) is independently mediated by a coupling reagent and optionally a coupling additive and/or a base.

Provided herein as Embodiment C. 195 is the method of Embodiment C. 194, wherein each coupling reagent is independently selected from carbodiimides, aminium salts, phosphonium salts, phosphonic acid anhydrides, phosphate-type coupling reagents, and combinations of any of the foregoing.

Provided herein as Embodiment C. 196 is the method of Embodiment C. 194 or Embodiment C. 195, wherein each coupling reagent is independently selected from N,N′-diisopropylcarbodiimide (DIC), N,N′-dicyclohexylcarbodiimide (DCC), 1-ethyl-3-(3-dimethylaminopropyl) carbodiimide (EDC), O-(1H-benzotriazol-1-yl)-N,N,N′,N′-tetramethyluronium hexafluorophosphate (HBTU), O-(1H-benzotriazol-1-yl)-N,N,N′,N′-tetramethyluronium tetrafluoroborate (TBTU), O-(7-azabenzotriazol-1-yl)-N,N,N′,N′-tetramethyluronium hexafluorophosphate (HATU), O-(6-chlorobenzotriazol-1-yl)-N,N,N′,N′-tetramethyluronium hexafluorophosphate (HCTU), O-(7-azabenzotriazol-1-yl)-N,N,N′,N′-tetramethyluronium tetrafluoroborate (TATU), O-(6-chlorobenzotriazol-1-yl)-N,N,N′,N′-tetramethyluronium tetrafluoroborate (TCTU), 2-(1H-benzotriazol-1-yl)-1,1,3,3-tetramethylaminium nitrate (TNTU), N,N,N′,N′-tetramethyl-O—(N-succinimidyl) uronium tetrafluoroborate (TSTU), 1-[(1-(cyano-2-ethoxy-2-oxoethylideneaminooxy)dimethylaminomorpholino)]uronium hexafluorophosphate (COMU), O-[(ethoxycarbonyl) cyanomethylenamino]-N,N,N′,N′-tetramethyluronium tetrafluoroborate (TOTU), (benzotriazol-1-yloxy)tripyrrolidinophosphonium hexafluorophosphate (PyBOP), (7-azabenzotriazol-1-yloxy)tripyrrolidinophosphonium hexafluorophosphate (PyAOP), bromotripyrrolidinophosphonium hexafluorophosphate (PyBroP), chlorotripyrrolidinophosphonium hexafluorophosphate (PyCIOP), (benzotriazol-1-yloxy)tris(dimethylamino)phosphonium hexafluorophosphate (BOP), bis(2-oxo-3-oxazolidinyl)phosphinic chloride (BOP—Cl), (ethyl cyano(hydroxyimino)acetato-O2)tri(1-pyrrolidinyl)phosphonium hexafluorophosphate (PyOxim), propanephosphonic acid anhydride (T3P), 3-(diethoxyphosphoryloxy)-1,2,3-benzotriazin-4 (3H)-one (DEPBT), and combinations of any of the foregoing.

Provided herein as Embodiment C. 197 is the method of any one of Embodiments C.190 or C.194 to C.196, wherein the coupling of any of step (A), step (B), or step (C) is independently mediated by a coupling reagent, a coupling additive, and optionally a base.

Provided herein as Embodiment C. 198 is the method of any one of Embodiments C.194 to C. 197, wherein each coupling additive is independently selected from 4-(dimethylamino)pyridine (DMAP), 1-hydroxybenzotriazole (HOBt), 1-hydroxy-6-chlorobenzotriazole (6-Cl—HOBt), 1-hydroxy-7-azabenzotriazole (HOAt), 2-pyridinol 1-oxide (HOPO), ethyl 2-cyano-2-(hydroxyimino)acetate (Oxyma), and combinations of any of the foregoing.

Provided herein as Embodiment C. 199 is the method of any one of Embodiments C.190 or C. 194 to C.198, wherein the coupling of any of step (A), step (B), or step (C) is independently mediated by a coupling reagent, a base, and optionally a coupling additive.

Provided herein as Embodiment C.200 is the method of any one of Embodiments C.194 to C. 199, wherein each base is independently selected from N,N-diisopropylethylamine (DIPEA), N-methylmorpholine (NMM), N-ethylmaleimide (NEM), triethylamine (TEA), 2,6-lutidine, 2,4,6-collidine, 2,2,6,6-tetramethylpiperidine (TMP), and combinations of any of the foregoing.

Provided herein as Embodiment C.201 is the method of any one of Embodiments C.190 or C.194 to C.200, wherein the coupling of any of step (A), step (B), or step (C) is independently mediated by DIC/Oxyma, DIC/HOPO, EDC/DMAP, EDC/HOBt/2,4,6-collidine, HATU/2,4,6-collidine, HBTU/2,4,6-collidine, HCTU/2,4,6-collidine, PyBOP/2,4,6-collidine, PyBOP/TEA, PyOxim/Oxyma, PyOxim/Oxyma/2,4,6-collidine, T3P/Oxyma, T3P/Oxyma/2,4,6-collidine, or TBTU/2,4,6-collidine.

Provided herein as Embodiment C.202 is the method of any one of Embodiments C. 190 or C.194 to C.201, wherein the coupling of any of step (A), step (B), or step (C) is conducted at a temperature in the range of 10° C. to 60° C.

Provided herein as Embodiment C.203 is the method of any one of Embodiments C.190 or C.194 to C.202, wherein the coupling of any of step (A), step (B), or step (C) is conducted for a time in the range of 1 hour to 72 hours.

Provided herein as Embodiment C.204 is the method of any one of Embodiments C.190 or C.194 to C.203, wherein the coupling of any of step (A), step (B), or step (C) is conducted in a mixture comprising an organic solvent.

Provided herein as Embodiment C.205 is the method of any one of Embodiments C.190 or C. 194 to C.204, wherein the coupling of any of step (A), step (B), or step (C) is conducted in a solution comprising an organic solvent.

Provided herein as Embodiment C.206 is the method of any one of Embodiments C. 190 or C. 194 to C.205, wherein the coupling of any of step (A), step (B), or step (C) is independently mediated by DIC/Oxyma, DIC/HOPO, EDC/DMAP, EDC/HOBt/2,4,6-collidine, HATU/2,4,6-collidine, HBTU/2,4,6-collidine, HCTU/2,4,6-collidine, PyBOP/2,4,6-collidine, PyBOP/TEA, PyOxim/Oxyma, PyOxim/Oxyma/2,4,6-collidine, T3P/Oxyma, T3P/Oxyma/2,4,6-collidine, or TBTU/2,4,6-collidine, and further wherein the coupling is conducted at a temperature in the range of 10° C. to 60° C. for a time in the range of 1 hour to 72 hours in a solution comprising an organic solvent.

Provided herein as Embodiment C.207 is the method of any one of Embodiments C.190 or C.194 to C.206, wherein the method comprises coupling a protected variant of Fragment 5 with a protected variant of Fragment 6 in solution to form a protected variant of Peptide B, wherein:
the N-terminus, the side chain of the Trp residue at position 2, and the side chain of the Lys residue at position 5 of the protected variant of Fragment 5 are protected and the protected variant of Fragment 5 has a free carboxyl group at the C-terminus; and
the side chain of the Ser residue at position 5, the side chain of the Ser residue at position 10, the side chain of the Ser residue at position 15, and the side chain of the Lys residue at position 16 of the protected variant of Fragment 6 are protected, the protected variant of Fragment 6 has a free amino group at the N-terminus, and the protected variant of Fragment 6 does not have a free carboxyl group at the C-terminus.

Provided herein as Embodiment C.208 is the method of Embodiment C.207, wherein the coupling is mediated by a coupling reagent and a base.

Provided herein as Embodiment C.209 is the method of Embodiment C.207 or Embodiment C.208, wherein the coupling is mediated by 1 to 10 equivalents of a coupling reagent and 1 to 10 equivalents of a base, wherein equivalents are relative to the protected variant of Fragment 5.

Provided herein as Embodiment C.210 is the method of Embodiment C.208 or Embodiment C.209, wherein the coupling reagent comprises benzotriazol-1-yloxytripyrrolidinophosphonium hexafluorophosphate (PyBOP) and the base comprises triethylamine.

Provided herein as Embodiment C.211 is the method of any one of Embodiments C.207 to C.210, wherein the coupling is conducted at a temperature in the range of 20° C. to 50° C. in a solution comprising dimethyl sulfoxide (DMSO) and methanol (MeOH).

Provided herein as Embodiment C.212 is the method of any one of Embodiments C.207 to C.211, wherein the coupling is mediated by 1 to 10 equivalents of PyBOP and 1 to 10 equivalents of triethylamine, wherein equivalents are relative to the protected variant of Fragment 5, and further wherein the coupling is conducted at a temperature in the range of 20° C. to 40° C. in a solution comprising DMSO and MeOH.

Provided herein as Embodiment C.213 is the method of Embodiment C.211 or Embodiment C.212, wherein the solution comprises 1-5:1 DMSO:MeOH.

Provided herein as Embodiment C.214 is the method of any one of Embodiments C.207 to C.213, wherein:
the side chain of the Trp residue at position 2 and the side chain of the Lys residue at position 5 of the protected variant of Fragment 5 are each protected with a tert-butyloxycarbonyl (Boc) protecting group; and
the Ser residues at position 5, position 10, and position 15 of the protected variant of Fragment 6 are each protected as a dimethylated pseudoproline (Psi (Me,Me) pro) moiety in which the oxazolidine is derived from Ser and the side chain of the Lys residue at position 16 of the protected variant of Fragment 6 is protected with an ivDde (1-(4,4-dimethyl-2,6-dioxocyclohex-1-ylidene)-3-methylbutyl) protecting group.

Provided herein as Embodiment C.215 is the method of any one of Embodiments C. 190 or C.194 to C.214, wherein the protected variant of Fragment 5 is Trt-Ala-Trp(Boc)-Leu-Val-Lys(Boc)-Gly-Gly-Gly-OH (SEQ ID NO: 59) and the protected variant of Fragment 6 is H-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130).

Provided herein as Embodiment C.216 is the method of any one of Embodiments C.190 or C.194 to C.215, wherein the method comprises coupling a protected variant of Fragment 1 with a protected variant of Fragment 2B in solution to form a protected variant of Peptide D, wherein:
the N-terminus, the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, and the side chain of the Ser residue at position 12 of the protected variant of Fragment 1 are protected and the protected variant of Fragment 1 has a free carboxyl group at the C-terminus; and
the side chain of the Tyr residue at position 1, the side chain of the Glu residue at position 3, the side chain of the Glu residue at position 4, the side chain of the Gln residue at position 5, the side chain of the Lys residue at position 8, and the side chain of the Glu residue at position 9 of the protected variant of Fragment 2B are protected and the protected variant of Fragment 2B has a free amino group at the N-terminus.

Provided herein as Embodiment C.217 is the method of Embodiment C.216, wherein the coupling is mediated by a coupling reagent, a coupling additive, and a base.

Provided herein as Embodiment C.218 is the method of Embodiment C.216 or Embodiment C.217, wherein the coupling is mediated by 1 to 10 equivalents of a coupling reagent, 1 to 5 equivalents of a coupling additive, and 1 to 10 equivalents of a base, wherein equivalents are relative to the protected variant of Fragment 2B.

Provided herein as Embodiment C.219 is the method of any one of Embodiments C.216 to C.218, wherein the coupling is mediated by 1 to 5 equivalents of PyBOP and 1 to 5 equivalents of 2,4,6-collidine, wherein equivalents are relative to the protected variant of Fragment 2B.

Provided herein as Embodiment C.220 is the method of any one of Embodiments C.216 to C.219, wherein the coupling is performed at a temperature in the range of 20° C. to 50° C. in a solution comprising tetrahydrofuran (THF) and dimethyl sulfoxide (DMSO).

Provided herein as Embodiment C.221 is the method of any one of Embodiments C.216 to C.220, wherein the coupling is mediated by 1 to 5 equivalents of PyBOP and 1 to 5 equivalents of 2,4,6-collidine, wherein equivalents are relative to the protected variant of Fragment 2B, and further wherein the coupling is performed at a temperature in the range of 30° C. to 50° C. in a solution comprising THF and DMSO.

Provided herein as Embodiment C.222 is the method of Embodiment C.220 or Embodiment C.221, wherein the solution comprises 5-8:1 THF:DMSO.

Provided herein as Embodiment C.223 is the method of any one of Embodiments C.207 to C.222, wherein:
the side chain of the His residue at position 1 of the protected variant of Fragment 1 is protected with a trityl (Trt) protecting group; the side chain of the Glu residue at position 3 of the protected variant of Fragment 1 is protected with a tert-butyl ester (OtBu) protecting group; the side chains of the Thr residues at position 5 and position 7 of the protected variant of Fragment 1 are each protected with a tert-butyl (tBu) protecting group; the side chains of the Ser residues at position 8 and position 11 of the protected variant of Fragment 1 are each protected with a tBu protecting group; the side chain of the Asp residue at position 9 of the protected variant of Fragment 1 is protected with an OtBu protecting group; the side chain of the Tyr residue at position 10 of the protected variant of Fragment 1 is protected with a tBu protecting group; and the Ser residue at position 12 of the protected variant of Fragment 1 is protected as a dimethylated pseudoproline (Psi (Me,Me) pro) moiety in which the oxazolidine is derived from Ser; and
the side chain of the Tyr residue at position 1 of the protected variant of Fragment 2B is protected with a tBu protecting group; the side chains of the Glu residues at position 3, position 4, and position 9 of the protected variant of Fragment 2B are each protected with a OtBu protecting group; the side chain of the Gln residue at position 5 of the protected variant of Fragment 2B is protected with a Trt protecting group; and the side chain of the Lys residue at position 8 of the protected variant of Fragment 2B is protected with a tert-butyloxycarbonyl (Boc) protecting group.

Provided herein as Embodiment C.224 is the method of any one of Embodiments C.190 or C.194 to C.223, wherein the protected variant of Fragment 1 is Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 166) and the protected variant of Fragment 2B is H-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 30).

Provided herein as Embodiment C.225 is the method of any one of Embodiments C.190 or C.194 to C.224, wherein the method comprises coupling a protected variant of Peptide B with a protected variant of Peptide D in solution to form a protected variant of Peptide A, wherein:
the side chain of the Trp residue at position 2, the side chain of the Lys residue at position 5, the side chain of the Ser residue at position 13, the side chain of the Ser residue at position 18, the side chain of the Ser residue at position 23, and the side chain of the Lys residue at position 24 of the protected variant of Peptide B are protected, the protected variant of Peptide B has a free amino group at the N-terminus, and the protected variant of Peptide B does not have a free carboxyl group at the C-terminus; and
the N-terminus, the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, the side chain of the Ser residue at position 12, the side chain of the Tyr residue at position 13, the side chain of the Glu residue at position 15, the side chain of the Glu residue at position 16, the side chain of the Gln residue at position 17, the side chain of the Lys residue at position 20, and the side chain of the Glu residue at position 21 of the protected variant of Peptide D are protected and the protected variant of Peptide D has a free carboxyl group at the C-terminus.

Provided herein as Embodiment C.226 is the method of Embodiment C.225, wherein the coupling is mediated by a coupling reagent, a coupling additive, and a base.

Provided herein as Embodiment C.227 is the method of Embodiment C.225 or Embodiment C.226, wherein the coupling is mediated by 1 to 10 equivalents of a coupling reagent, 1 to 10 equivalents of a coupling additive, and 1 to 10 equivalents of a base, wherein equivalents are relative to the protected variant of Peptide D.

Provided herein as Embodiment C.228 is the method of any one of Embodiments C.225 to C.227, wherein the coupling is mediated by 1 to 10 equivalents of 1-ethyl-3-(3-dimethylaminopropyl) carbodiimide (EDC), 1 to 10 equivalents of 1-hydroxybenzotriazole (HOBt), and 1 to 10 equivalents of 2,4,6-collidine, wherein equivalents are relative to the protected variant of Peptide D.

Provided herein as Embodiment C.229 is the method of any one of Embodiments C.225 to C.228, wherein the coupling is conducted at a temperature in the range of 20° C. to 50° C. in a solution comprising tetrahydrofuran (THF) and water (H₂O).

Provided herein as Embodiment C.230 is the method of any one of Embodiments C.225 to C.229, wherein the coupling is mediated by 1 to 10 equivalents of EDC, 1 to 10 equivalents of HOBt, and 1 to 10 equivalents of 2,4,6-collidine, wherein equivalents are relative to the protected variant of Peptide D, and further wherein the coupling is conducted at a temperature in the range of 20° C. to 40° C. in a solution comprising THF and H₂O.

Provided herein as Embodiment C.231 is the method of Embodiment C.229 or Embodiment C.230, wherein the solution comprises 3-5:1 THF:H₂O.

Provided herein as Embodiment C.232 is the method of any one of Embodiments C.190 or C.194 to C.231, wherein:
the side chain of the Trp residue at position 2 and the side chain of the Lys residue at position 5 of the protected variant of Peptide B are each protected with a tert-butyloxycarbonyl (Boc) protecting group, the Ser residues at position 13, position 18, and position 23 of the protected variant of Peptide B are each protected as a dimethylated pseudoproline (Psi (Me, Me) pro) moiety in which the oxazolidine is derived from Ser, and the side chain of the Lys residue at position 24 of the protected variant of Peptide B is protected with an ivDde (1-(4,4-dimethyl-2,6-dioxocyclohex-1-ylidene)-3-methylbutyl) protecting group; and
the side chain of the His residue at position 1 of Peptide D is protected with a trityl (Trt) protecting group; the side chains of the Glu residues at position 3, position 15, position 16, and position 21 of Peptide D are each protected with a tert-butyl ester (OtBu) protecting group; the side chains of the Thr residues at position 5 and position 7 of Peptide D are each protected with a tert-butyl (tBu) protecting group; the side chains of the Ser residues at position 8 and position 11 of Peptide D are each protected with a tBu protecting group; the side chain of the Asp residue at position 9 of Peptide D is protected with an OtBu protecting group; the side chains of the Tyr residues at positions 10 and 13 of Peptide D are each protected with a tBu protecting group; the Ser residue at position 12 of Peptide D is protected as a dimethylated pseudoproline (Psi (Me,Me) pro) moiety in which the oxazolidine is derived from Ser; the side chain of the Gln residue at position 17 of Peptide D is protected with a Trt protecting group; and the side chain of the Lys residue at position 20 of Peptide D is protected with a tert-butyloxycarbonyl (Boc) protecting group.

Provided herein as Embodiment C.233 is the method of any one of Embodiments C. 190 or C.194 to C.232, wherein the protected variant of Peptide B and the protected variant of Peptide D that are coupled to form the protected variant of Peptide A are H-Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 147) and Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 169), respectively.

Provided herein as Embodiment C.234 is the method of Embodiment C. 190, the method comprising:
(A) (i) coupling or having coupled a protected variant of Fragment 5 with a protected variant of Fragment 6 to form a first protected variant of Peptide B, wherein the protected variant of Fragment 5 has a free carboxyl group at the C-terminus and a protected N-terminus, the protected variant of Fragment 6 has a free amino group at the N-terminus, Fragment 5 has the amino acid sequence of SEQ ID NO: 5, Fragment 6 has the amino acid sequence of SEQ ID NO: 6, and Peptide B has the amino acid sequence of SEQ ID NO: 9; and (ii) selectively deprotecting or having selectively deprotected the N-terminus of the first protected variant of Peptide B from step (A) (i) to form a second protected variant of Peptide B with a free amino group at the N-terminus; and
(B) coupling or having coupled a protected variant of Fragment 1 with a protected variant of Fragment 2B to form a protected variant of Peptide D, wherein the protected variant of Fragment 1 has a free carboxyl group at the C-terminus and a protected N-terminus, the protected variant of Fragment 2B has a free amino group at the N-terminus, Fragment 1 has the amino acid sequence of SEQ ID NO: 1, Fragment 2B has the amino acid sequence of SEQ ID NO: 16, and Peptide D has the amino acid sequence of SEQ ID NO: 15; and
(C) coupling or having coupled the second protected variant of Peptide B from step (A) with the protected variant of Peptide D from step (B) to form a protected variant of Peptide A, wherein Peptide A has the amino acid sequence of SEQ ID NO: 7.

Provided herein as Embodiment C.235 is the method of Embodiment C.234, wherein:
the side chain of the Trp residue at position 2 and the side chain of the Lys residue at position 5 of the protected variant of Fragment 5 are protected;
the side chain of the Ser residue at position 5, the side chain of the Ser residue at position 10, the side chain of the Ser residue at position 15, and the side chain of the Lys residue at position 16 of the protected variant of Fragment 6 are protected, and the protected variant of Fragment 6 does not have a free carboxyl group at the C-terminus;
the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, and the side chain of the Ser residue at position 12 of the protected variant of Fragment 1 are protected; and
the side chain of the Tyr residue at position 1, the side chain of the Glu residue at position 3, the side chain of the Glu residue at position 4, the side chain of the Gln residue at position 5, the side chain of the Lys residue at position 8, and the side chain of the Glu residue at position 9 of the protected variant of Fragment 2B are protected.

Provided herein as Embodiment C.236 is the method of Embodiment C.234 or Embodiment C.235, wherein:
the side chain of the Trp residue at position 2 and the side chain of the Lys residue at position 5 of the protected variant of Fragment 5 are each protected with a tert-butyloxycarbonyl (Boc) protecting group;
the Ser residues at position 5, position 10, and position 15 of the protected variant of Fragment 6 are each protected as a dimethylated pseudoproline (Psi (Me,Me) pro) moiety in which the oxazolidine is derived from Ser and the side chain of the Lys residue at position 16 of the protected variant of Fragment 6 is protected with an ivDde (1-(4,4-dimethyl-2,6-dioxocyclohex-1-ylidene)-3-methylbutyl) protecting group;
the side chain of the His residue at position 1 of the protected variant of Fragment 1 is protected with a trityl (Trt) protecting group; the side chain of the Glu residue at position 3 of the protected variant of Fragment 1 is protected with a tert-butyl ester (OtBu) protecting group; the side chains of the Thr residues at position 5 and position 7 of the protected variant of Fragment 1 are each protected with a tert-butyl (tBu) protecting group; the side chains of the Ser residues at position 8 and position 11 of the protected variant of Fragment 1 are each protected with a tBu protecting group; the side chain of the Asp residue at position 9 of the protected variant of Fragment 1 is protected with an OtBu protecting group; the side chain of the Tyr residue at position 10 of the protected variant of Fragment 1 is protected with a tBu protecting group; and the Ser residue at position 12 of the protected variant of Fragment 1 is protected as a dimethylated pseudoproline (Psi (Me,Me) pro) moiety in which the oxazolidine is derived from Ser; and
the side chain of the Tyr residue at position 1 of the protected variant of Fragment 2B is protected with a tBu protecting group; the side chains of the Glu residues at position 3, position 4, and position 9 of the protected variant of Fragment 2B are each protected with a OtBu protecting group; the side chain of the Gln residue at position 5 of the protected variant of Fragment 2B is protected with a Trt protecting group; and the side chain of the Lys residue at position 8 of the protected variant of Fragment 2B is protected with a tert-butyloxycarbonyl (Boc) protecting group.

Provided herein as Embodiment C.237 is the method of any one of Embodiments C.234 to C.236, wherein:
the protected variant of Fragment 5 is Trt-Ala-Trp(Boc)-Leu-Val-Lys(Boc)-Gly-Gly-Gly-OH (SEQ ID NO: 59);
the protected variant of Fragment 6 is H-Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130);
the protected variant of Fragment 1 is Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 166); and
the protected variant of Fragment 2B is H-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 30).

Provided herein as Embodiment C.238 is the method of any one of Embodiments C.234 to C.237, wherein:
the first protected variant of Peptide B is Trt-Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 170);
the second protected variant of Peptide B is H-Ala-Trp(Boc)-Leu-Val-Lys(Boc)-Gly-Gly-Gly-Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 147); and
the protected variant of Peptide D is Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 169).

Provided herein as Embodiment C.239 is the method of any one of Embodiments C.234 to C.238, wherein the protected variant of Peptide A is Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser (Psi(Me,Me)Pro)-Lys(ivDde)-NH₂ (SEQ ID NO: 171).

Provided herein as Embodiment C.240 is the method of any one of Embodiments C.190 to C.239, wherein the method further comprises bromoacetylating an &-amino group of the C-terminal Lys residue of a protected variant of Peptide A to form a bromoacetylated protected variant of Peptide A, optionally wherein the bromoacetylating comprises selectively deprotecting the side chain of the C-terminal Lys residue of the protected variant of Peptide A to form a partially deprotected variant of Peptide A and then coupling the partially deprotected variant of Peptide A with bromoacetic acid or bromoacetic anhydride to form a bromoacetylated protected variant of Peptide A, wherein the coupling is mediated by a coupling reagent and optionally a coupling additive and/or a base.

Provided herein as Embodiment C.241 is the method of Embodiment C.240, wherein the selectively deprotecting comprises the method according to any one of Embodiments 160 to 166.

Provided herein as Embodiment C.242 is the method of Embodiment C.240 or Embodiment C.241, wherein the coupling comprises the method according to any one of Embodiments 167 to 179.

Provided herein as Embodiment C.243 is the method of any one of Embodiments C.240 to C.242, wherein the method further comprises contacting the bromoacetylated protected variant of Peptide A with a Brønsted acid or a Lewis acid, optionally in the presence of a scavenger, to form a bromoacetylated variant of Peptide A, wherein the bromoacetylated variant of Peptide A has the amino acid sequence of SEQ ID NO: 1, the N-terminus and the side chains of the amino acids at positions 1-46 of the bromoacetylated variant of Peptide A are not protected, the C-terminal Lys residue is bromoacetylated, and the C-terminus of the bromoacetylated variant of Peptide A is amidated as a C-terminal primary amide.

Provided herein as Embodiment C.244 is the method of Embodiment C.243, wherein the contacting comprises the method according to any one of Embodiments 180 to 189.

Provided herein as Embodiment C.245 is a molecule selected from:

(SEQ ID NO: 166)
Boc-His(Trt)-Aib-Glu(OtBu)-Gly-Thr(tBu)-Phe-
Thr(tBu)-Ser(tBu)-Asp(OtBu)-Tyr(tBu)-Ser(tBu)-
Ser(Psi(Me,Me)Pro)-OH;
(SEQ ID NO: 30)
H-Tyr(tBu)-Leu-Glu(OtBu)-Glu(OtBu)-Gln(Trt)-
Ala-Ala-Lys(Boc)-Glu(OtBu)-Phe-Ile-OH;
(SEQ ID NO: 169)
Boc-His(Trt)-Aib-Glu(OtBu)-Gly-Thr(tBu)-Phe-
Thr(tBu)-Ser(tBu)-Asp(OtBu)-Tyr(tBu)-Ser(tBu)-
Ser(Psi(Me,Me)Pro)-Tyr(tBu)-Leu-Glu(OtBu)-
Glu(OtBu)-Gln(Trt)-Ala-Ala-Lys(Boc)-Glu(OtBu)-
Phe-Ile-OH;
(SEQ ID NO: 59)
Trt-Ala-Trp(Boc)-Leu-Val-Lys(Boc)-Gly-Gly-Gly-OH;
(SEQ ID NO: 130)
H-Gly-Gly-Gly-Gly-Ser(Psi(Me,Me)Pro)-Gly-Gly-
Gly-Gly-Ser(Psi(Me,Me)Pro)-Gly-Gly-Gly-Gly-
Ser(Psi(Me,Me)Pro)-Lys(ivDde)-NH₂;
(SEQ ID NO: 170)
Trt-Ala-Trp(Boc)-Leu-Val-Lys(Boc)-Gly-Gly-Gly-
Gly-Gly-Gly-Gly-Ser(Psi(Me,Me)Pro)-Gly-Gly-Gly-
Gly-Ser(Psi(Me,Me)Pro)-Gly-Gly-Gly-Gly-
Ser(Psi(Me,Me)Pro)-Lys(ivDde)-NH₂;
(SEQ ID NO: 147)
H-Ala-Trp(Boc)-Leu-Val-Lys(Boc)-Gly-Gly-Gly-
Gly-Gly-Gly-Gly-Ser(Psi(Me,Me)Pro)-Gly-Gly-
Gly-Gly-Ser(Psi(Me,Me)Pro)-Gly-Gly-Gly-Gly-
Ser(Psi(Me,Me)Pro)-Lys(ivDde)-NH₂;
and
(SEQ ID NO: 171)
Boc-His(Trt)-Aib-Glu(OtBu)-Gly-Thr(tBu)-Phe-
Thr(tBu)-Ser(tBu)-Asp(OtBu)-Tyr(tBu)-Ser(tBu)-
Ser(Psi(Me,Me)Pro)-Tyr(tBu)-Leu-Glu(OtBu)-
Glu(OtBu)-Gln(Trt)-Ala-Ala-Lys(Boc)-Glu(OtBu)-
Phe-Ile-Ala-Trp(Boc)-Leu-Val-Lys(Boc)-Gly-Gly-
Gly-Gly-Gly-Gly-Gly-Ser(Psi(Me,Me)Pro)-Gly-Gly-
Gly-Gly-Ser(Psi(Me,Me)Pro)-Gly-Gly-Gly-Gly-
Ser(Psi(Me,Me)Pro)-Lys(ivDde)-NH₂,

or a salt thereof.

What is claimed is:

1. A method of preparing Peptide A or a variant thereof, comprising:
(A) coupling a protected variant of Fragment 5 with a protected variant of Fragment 6 to form a protected variant of Peptide B; and/or
(B) coupling a protected variant of Fragment 1 with a protected variant of Fragment 2B to form a protected variant of Peptide D; and/or
(C) coupling a protected variant of Peptide B with a protected variant of Peptide D to form a protected variant of Peptide A,
wherein Peptide A has the amino acid sequence of SEQ ID NO: 7, Peptide B has the amino acid sequence of SEQ ID NO: 9, Peptide D has the amino acid sequence of SEQ ID NO: 15, Fragment 1 has the amino acid sequence of SEQ ID NO: 1, Fragment 2B has the amino acid sequence of SEQ ID NO: 16, Fragment 5 has the amino acid sequence of SEQ ID NO: 5, and Fragment 6 has the amino acid sequence of SEQ ID NO: 6.

2. The method of claim 1, wherein the coupling of any of step (A), step (B), or step (C) is independently mediated by a coupling reagent and optionally a coupling additive and/or a base.

3. The method of claim 2, wherein each coupling reagent is independently selected from carbodiimides, aminium salts, phosphonium salts, phosphonic acid anhydrides, phosphate-type coupling reagents, and combinations of any of the foregoing.

4. The method of claim 2 or claim 3, wherein each coupling reagent is independently selected from N,N′-diisopropylcarbodiimide (DIC), N,N′-dicyclohexylcarbodiimide (DCC), 1-ethyl-3-(3-dimethylaminopropyl) carbodiimide (EDC), O-(1H-benzotriazol-1-yl)-N,N,N′,N′-tetramethyluronium hexafluorophosphate (HBTU), O-(1H-benzotriazol-1-yl)-N,N,N′,N′-tetramethyluronium tetrafluoroborate (TBTU), O-(7-azabenzotriazol-1-yl)-N,N,N′,N′-tetramethyluronium hexafluorophosphate (HATU), O-(6-chlorobenzotriazol-1-yl)-N,N,N′,N′-tetramethyluronium hexafluorophosphate (HCTU), O-(7-azabenzotriazol-1-yl)-N,N,N′,N′-tetramethyluronium tetrafluoroborate (TATU), O-(6-chlorobenzotriazol-1-yl)-N,N,N′,N′-tetramethyluronium tetrafluoroborate (TCTU), 2-(1H-benzotriazol-1-yl)-1,1,3,3-tetramethylaminium nitrate (TNTU), N,N,N′,N′-tetramethyl-O—(N-succinimidyl) uronium tetrafluoroborate (TSTU), 1-[(1-(cyano-2-ethoxy-2-oxoethylideneaminooxy)dimethylaminomorpholino)]uronium hexafluorophosphate (COMU), O-[(ethoxycarbonyl) cyanomethylenamino]-N,N,N′,N′-tetramethyluronium tetrafluoroborate (TOTU), (benzotriazol-1-yloxy)tripyrrolidinophosphonium hexafluorophosphate (PyBOP), (7-azabenzotriazol-1-yloxy)tripyrrolidinophosphonium hexafluorophosphate (PyAOP), bromotripyrrolidinophosphonium hexafluorophosphate (PyBroP), chlorotripyrrolidinophosphonium hexafluorophosphate (PyCIOP), (benzotriazol-1-yloxy)tris(dimethylamino)phosphonium hexafluorophosphate (BOP), bis(2-oxo-3-oxazolidinyl)phosphinic chloride (BOP—Cl), (ethyl cyano(hydroxyimino)acetato-O²)tri(1-pyrrolidinyl)phosphonium hexafluorophosphate (PyOxim), propanephosphonic acid anhydride (T3P), 3-(diethoxyphosphoryloxy)-1,2,3-benzotriazin-4 (3H)-one (DEPBT), and combinations of any of the foregoing.

5. The method of any one of claims 1 to 4, wherein the coupling of any of step (A), step (B), or step (C) is independently mediated by a coupling reagent, a coupling additive, and optionally a base.

6. The method of any one of claims 2 to 5, wherein each coupling additive is independently selected from 4-(dimethylamino)pyridine (DMAP), 1-hydroxybenzotriazole (HOBt), 1-hydroxy-6-chlorobenzotriazole (6-Cl—HOBt), 1-hydroxy-7-azabenzotriazole (HOAt), 2-pyridinol 1-oxide (HOPO), ethyl 2-cyano-2-(hydroxyimino)acetate (Oxyma), and combinations of any of the foregoing.

7. The method of any one of claims 1 to 6, wherein the coupling of any of step (A), step (B), or step (C) is independently mediated by a coupling reagent, a base, and optionally a coupling additive.

8. The method of any one of claims 2 to 7, wherein each base is independently selected from N,N-diisopropylethylamine (DIPEA), N-methylmorpholine (NMM), N-ethylmaleimide (NEM), triethylamine (TEA), 2,6-lutidine, 2,4,6-collidine, 2,2,6,6-tetramethylpiperidine (TMP), and combinations of any of the foregoing.

9. The method of any one of claims 1 to 8, wherein the coupling of any of step (A), step (B), or step (C) is independently mediated by DIC/Oxyma, DIC/HOPO, EDC/DMAP, EDC/HOBt/2,4,6-collidine, HATU/2,4,6-collidine, HBTU/2,4,6-collidine, HCTU/2,4,6-collidine, PyBOP/2,4,6-collidine, PyBOP/TEA, PyOxim/Oxyma, PyOxim/Oxyma/2,4,6-collidine, T3P/Oxyma, T3P/Oxyma/2,4,6-collidine, or TBTU/2,4,6-collidine.

10. The method of any one of claims 1 to 9, wherein the coupling of any of step (A), step (B), or step (C) is conducted at a temperature in the range of 10° C. to 60° C.

11. The method of any one of claims 1 to 10, wherein the coupling of any of step (A), step (B), or step (C) is conducted for a time in the range of 1 hour to 72 hours.

12. The method of any one of claims 1 to 11, wherein the coupling of any of step (A), step (B), or step (C) is conducted in a mixture comprising an organic solvent.

13. The method of any one of claims 1 to 12, wherein the coupling of any of step (A), step (B), or step (C) is conducted in a solution comprising an organic solvent.

14. The method of any one of claims 1 to 13, wherein the coupling of any of step (A), step (B), or step (C) is independently mediated by DIC/Oxyma, DIC/HOPO, EDC/DMAP, EDC/HOBt/2,4,6-collidine, HATU/2,4,6-collidine, HBTU/2,4,6-collidine, HCTU/2,4,6-collidine, PyBOP/2,4,6-collidine, PyBOP/TEA, PyOxim/Oxyma, PyOxim/Oxyma/2,4,6-collidine, T3P/Oxyma, T3P/Oxyma/2,4,6-collidine, or TBTU/2,4,6-collidine, and further wherein the coupling is conducted at a temperature in the range of 10° C. to 60° C. for a time in the range of 1 hour to 72 hours in a solution comprising an organic solvent.

15. The method of any one of claims 1 to 14, wherein the method comprises coupling a protected variant of Fragment 5 with a protected variant of Fragment 6 in solution to form a protected variant of Peptide B, wherein:
the N-terminus, the side chain of the Trp residue at position 2, and the side chain of the Lys residue at position 5 of the protected variant of Fragment 5 are protected and the protected variant of Fragment 5 has a free carboxyl group at the C-terminus; and
the side chain of the Ser residue at position 5, the side chain of the Ser residue at position 10, the side chain of the Ser residue at position 15, and the side chain of the Lys residue at position 16 of the protected variant of Fragment 6 are protected, the protected variant of Fragment 6 has a free amino group at the N-terminus, and the protected variant of Fragment 6 does not have a free carboxyl group at the C-terminus.

16. The method of claim 15, wherein the coupling is mediated by a coupling reagent and a base.

17. The method of claim 15 or claim 16, wherein the coupling is mediated by 1 to 10 equivalents of a coupling reagent and 1 to 10 equivalents of a base, wherein equivalents are relative to the protected variant of Fragment 5.

18. The method of claim 16 or claim 17, wherein the coupling reagent comprises benzotriazol-1-yloxytripyrrolidinophosphonium hexafluorophosphate (PyBOP) and the base comprises triethylamine.

19. The method of any one of claims 15 to 18, wherein the coupling is conducted at a temperature in the range of 20° C. to 50° C. in a solution comprising dimethyl sulfoxide (DMSO) and methanol (MeOH).

20. The method of any one of claims 15 to 19, wherein the coupling is mediated by 1 to 10 equivalents of PyBOP and 1 to 10 equivalents of triethylamine, wherein equivalents are relative to the protected variant of Fragment 5, and further wherein the coupling is conducted at a temperature in the range of 20° C. to 40° C. in a solution comprising DMSO and MeOH.

21. The method of claim 19 or claim 20, wherein the solution comprises 1-5:1 DMSO:MeOH.

22. The method of any one of claims 15 to 21, wherein:
the side chain of the Trp residue at position 2 and the side chain of the Lys residue at position 5 of the protected variant of Fragment 5 are each protected with a tert-butyloxycarbonyl (Boc) protecting group; and
the Ser residues at position 5, position 10, and position 15 of the protected variant of Fragment 6 are each protected as a dimethylated pseudoproline (Psi (Me,Me) pro) moiety in which the oxazolidine is derived from Ser and the side chain of the Lys residue at position 16 of the protected variant of Fragment 6 is protected with an ivDde (1-(4,4-dimethyl-2,6-dioxocyclohex-1-ylidene)-3-methylbutyl) protecting group.

23. The method of any one of claims 1 to 21, wherein the protected variant of Fragment 5 is Trt-Ala-Trp(Boc)-Leu-Val-Lys(Boc)-Gly-Gly-Gly-OH (SEQ ID NO: 59) and the protected variant of Fragment 6 is H-Gly-Gly-Gly-Gly-Ser(Psi(Me,Me)Pro)-Gly-Gly-Gly-Gly-Ser(Psi(Me, Me)Pro)-Gly-Gly-Gly-Gly-Ser(Psi(Me,Me)Pro)-Lys(ivDde)-NH₂ (SEQ ID NO: 130).

24. The method of any one of claims 1 to 23, wherein the method comprises coupling a protected variant of Fragment 1 with a protected variant of Fragment 2B in solution to form a protected variant of Peptide D, wherein:
the N-terminus, the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, and the side chain of the Ser residue at position 12 of the protected variant of Fragment 1 are protected and the protected variant of Fragment 1 has a free carboxyl group at the C-terminus; and
the side chain of the Tyr residue at position 1, the side chain of the Glu residue at position 3, the side chain of the Glu residue at position 4, the side chain of the Gln residue at position 5, the side chain of the Lys residue at position 8, and the side chain of the Glu residue at position 9 of the protected variant of Fragment 2B are protected and the protected variant of Fragment 2B has a free amino group at the N-terminus.

25. The method of claim 24, wherein the coupling is mediated by a coupling reagent, a coupling additive, and a base.

26. The method of claim 24 or claim 25, wherein the coupling is mediated by 1 to 10 equivalents of a coupling reagent, 1 to 5 equivalents of a coupling additive, and 1 to 10 equivalents of a base, wherein equivalents are relative to the protected variant of Fragment 2B.

27. The method of any one of claims 24 to 26, wherein the coupling is mediated by 1 to 5 equivalents of PyBOP and 1 to 5 equivalents of 2,4,6-collidine, wherein equivalents are relative to the protected variant of Fragment 2B.

28. The method of any one of claims 24 to 27, wherein the coupling is performed at a temperature in the range of 20° C. to 50° C. in a solution comprising tetrahydrofuran (THF) and dimethyl sulfoxide (DMSO).

29. The method of any one of claims 24 to 28, wherein the coupling is mediated by 1 to 5 equivalents of PyBOP and 1 to 5 equivalents of 2,4,6-collidine, wherein equivalents are relative to the protected variant of Fragment 2B, and further wherein the coupling is performed at a temperature in the range of 30° C. to 50° C. in a solution comprising THF and DMSO.

30. The method of claim 28 or claim 29, wherein the solution comprises 5-8:1 THF:DMSO.

31. The method of any one of claims 24 to 30, wherein:
the side chain of the His residue at position 1 of the protected variant of Fragment 1 is protected with a trityl (Trt) protecting group; the side chain of the Glu residue at position 3 of the protected variant of Fragment 1 is protected with a tert-butyl ester (OtBu) protecting group; the side chains of the Thr residues at position 5 and position 7 of the protected variant of Fragment 1 are each protected with a tert-butyl (tBu) protecting group; the side chains of the Ser residues at position 8 and position 11 of the protected variant of Fragment 1 are each protected with a tBu protecting group; the side chain of the Asp residue at position 9 of the protected variant of Fragment 1 is protected with an OtBu protecting group; the side chain of the Tyr residue at position 10 of the protected variant of Fragment 1 is protected with a tBu protecting group; and the Ser residue at position 12 of the protected variant of Fragment 1 is protected as a dimethylated pseudoproline (Psi (Me,Me) pro) moiety in which the oxazolidine is derived from Ser; and
the side chain of the Tyr residue at position 1 of the protected variant of Fragment 2B is protected with a tBu protecting group; the side chains of the Glu residues at position 3, position 4, and position 9 of the protected variant of Fragment 2B are each protected with a OtBu protecting group; the side chain of the Gln residue at position 5 of the protected variant of Fragment 2B is protected with a Trt protecting group; and the side chain of the Lys residue at position 8 of the protected variant of Fragment 2B is protected with a tert-butyloxycarbonyl (Boc) protecting group.

32. The method of any one of claims 1 to 31, wherein the protected variant of Fragment 1 is Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 166) and the protected variant of Fragment 2B is H-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 30).

33. The method of any one of claims 1 to 32, wherein the method comprises coupling a protected variant of Peptide B with a protected variant of Peptide D in solution to form a protected variant of Peptide A, wherein:
the side chain of the Trp residue at position 2, the side chain of the Lys residue at position 5, the side chain of the Ser residue at position 13, the side chain of the Ser residue at position 18, the side chain of the Ser residue at position 23, and the side chain of the Lys residue at position 24 of the protected variant of Peptide B are protected, the protected variant of Peptide B has a free amino group at the N-terminus, and the protected variant of Peptide B does not have a free carboxyl group at the C-terminus; and
the N-terminus, the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, the side chain of the Ser residue at position 12, the side chain of the Tyr residue at position 13, the side chain of the Glu residue at position 15, the side chain of the Glu residue at position 16, the side chain of the Gln residue at position 17, the side chain of the Lys residue at position 20, and the side chain of the Glu residue at position 21 of the protected variant of Peptide D are protected and the protected variant of Peptide D has a free carboxyl group at the C-terminus.

34. The method of claim 33, wherein the coupling is mediated by a coupling reagent, a coupling additive, and a base.

35. The method of claim 33 or claim 34, wherein the coupling is mediated by 1 to 10 equivalents of a coupling reagent, 1 to 10 equivalents of a coupling additive, and 1 to 10 equivalents of a base, wherein equivalents are relative to the protected variant of Peptide D.

36. The method of any one of claims 33 to 35, wherein the coupling is mediated by 1 to 10 equivalents of 1-ethyl-3-(3-dimethylaminopropyl) carbodiimide (EDC), 1 to 10 equivalents of 1-hydroxybenzotriazole (HOBt), and 1 to 10 equivalents of 2,4,6-collidine, wherein equivalents are relative to the protected variant of Peptide D.

37. The method of any one of claims 33 to 36, wherein the coupling is conducted at a temperature in the range of 20° C. to 50° C. in a solution comprising tetrahydrofuran (THF) and water (H₂O).

38. The method of any one of claims 33 to 37, wherein the coupling is mediated by 1 to 10 equivalents of EDC, 1 to 10 equivalents of HOBt, and 1 to 10 equivalents of 2,4,6-collidine, wherein equivalents are relative to the protected variant of Peptide D, and further wherein the coupling is conducted at a temperature in the range of 20° C. to 40° C. in a solution comprising THF and H₂O.

39. The method of claim 37 or claim 38, wherein the solution comprises 3-5:1 THF:H₂O.

40. The method of any one of claims 1 to 39, wherein:
the side chain of the Trp residue at position 2 and the side chain of the Lys residue at position 5 of the protected variant of Peptide B are each protected with a tert-butyloxycarbonyl (Boc) protecting group, the Ser residues at position 13, position 18, and position 23 of the protected variant of Peptide B are each protected as a dimethylated pseudoproline (Psi (Me, Me) pro) moiety in which the oxazolidine is derived from Ser, and the side chain of the Lys residue at position 24 of the protected variant of Peptide B is protected with an ivDde (1-(4,4-dimethyl-2,6-dioxocyclohex-1-ylidene)-3-methylbutyl) protecting group; and
the side chain of the His residue at position 1 of Peptide D is protected with a trityl (Trt) protecting group; the side chains of the Glu residues at position 3, position 15, position 16, and position 21 of Peptide D are each protected with a tert-butyl ester (OtBu) protecting group; the side chains of the Thr residues at position 5 and position 7 of Peptide D are each protected with a tert-butyl (tBu) protecting group; the side chains of the Ser residues at position 8 and position 11 of Peptide D are each protected with a tBu protecting group; the side chain of the Asp residue at position 9 of Peptide D is protected with an OtBu protecting group; the side chains of the Tyr residues at positions 10 and 13 of Peptide D are each protected with a tBu protecting group; the Ser residue at position 12 of Peptide D is protected as a dimethylated pseudoproline (Psi (Me,Me) pro) moiety in which the oxazolidine is derived from Ser; the side chain of the Gln residue at position 17 of Peptide D is protected with a Trt protecting group; and the side chain of the Lys residue at position 20 of Peptide D is protected with a tert-butyloxycarbonyl (Boc) protecting group.

41. The method of any one of claims 1 to 40, wherein the protected variant of Peptide B and the protected variant of Peptide D that are coupled to form the protected variant of Peptide A are H-Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 147) and Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 169), respectively.

42. The method of claim 1, the method comprising:
(A) (i) coupling or having coupled a protected variant of Fragment 5 with a protected variant of Fragment 6 to form a first protected variant of Peptide B, wherein the protected variant of Fragment 5 has a free carboxyl group at the C-terminus and a protected N-terminus, the protected variant of Fragment 6 has a free amino group at the N-terminus, Fragment 5 has the amino acid sequence of SEQ ID NO: 5, Fragment 6 has the amino acid sequence of SEQ ID NO: 6, and Peptide B has the amino acid sequence of SEQ ID NO: 9; and (ii) selectively deprotecting or having selectively deprotected the N-terminus of the first protected variant of Peptide B from step (A) (i) to form a second protected variant of Peptide B with a free amino group at the N-terminus; and
(B) coupling or having coupled a protected variant of Fragment 1 with a protected variant of Fragment 2B to form a protected variant of Peptide D, wherein the protected variant of Fragment 1 has a free carboxyl group at the C-terminus and a protected N-terminus, the protected variant of Fragment 2B has a free amino group at the N-terminus, Fragment 1 has the amino acid sequence of SEQ ID NO: 1, Fragment 2B has the amino acid sequence of SEQ ID NO: 16, and Peptide D has the amino acid sequence of SEQ ID NO: 15; and
(C) coupling or having coupled the second protected variant of Peptide B from step (A) with the protected variant of Peptide D from step (B) to form a protected variant of Peptide A, wherein Peptide A has the amino acid sequence of SEQ ID NO: 7.

43. The method of claim 42, wherein:
the side chain of the Trp residue at position 2 and the side chain of the Lys residue at position 5 of the protected variant of Fragment 5 are protected;
the side chain of the Ser residue at position 5, the side chain of the Ser residue at position 10, the side chain of the Ser residue at position 15, and the side chain of the Lys residue at position 16 of the protected variant of Fragment 6 are protected, and the protected variant of Fragment 6 does not have a free carboxyl group at the C-terminus;
the side chain of the His residue at position 1, the side chain of the Glu residue at position 3, the side chain of the Thr residue at position 5, the side chain of the Thr residue at position 7, the side chain of the Ser residue at position 8, the side chain of the Asp residue at position 9, the side chain of the Tyr residue at position 10, the side chain of the Ser residue at position 11, and the side chain of the Ser residue at position 12 of the protected variant of Fragment 1 are protected; and
the side chain of the Tyr residue at position 1, the side chain of the Glu residue at position 3, the side chain of the Glu residue at position 4, the side chain of the Gln residue at position 5, the side chain of the Lys residue at position 8, and the side chain of the Glu residue at position 9 of the protected variant of Fragment 2B are protected.

44. The method of claim 42 or claim 43, wherein:
the side chain of the Trp residue at position 2 and the side chain of the Lys residue at position 5 of the protected variant of Fragment 5 are each protected with a tert-butyloxycarbonyl (Boc) protecting group;
the Ser residues at position 5, position 10, and position 15 of the protected variant of Fragment 6 are each protected as a dimethylated pseudoproline (Psi (Me,Me) pro) moiety in which the oxazolidine is derived from Ser and the side chain of the Lys residue at position 16 of the protected variant of Fragment 6 is protected with an ivDde (1-(4,4-dimethyl-2,6-dioxocyclohex-1-ylidene)-3-methylbutyl) protecting group;
the side chain of the His residue at position 1 of the protected variant of Fragment 1 is protected with a trityl (Trt) protecting group; the side chain of the Glu residue at position 3 of the protected variant of Fragment 1 is protected with a tert-butyl ester (OtBu) protecting group; the side chains of the Thr residues at position 5 and position 7 of the protected variant of Fragment 1 are each protected with a tert-butyl (tBu) protecting group; the side chains of the Ser residues at position 8 and position 11 of the protected variant of Fragment 1 are each protected with a tBu protecting group; the side chain of the Asp residue at position 9 of the protected variant of Fragment 1 is protected with an OtBu protecting group; the side chain of the Tyr residue at position 10 of the protected variant of Fragment 1 is protected with a tBu protecting group; and the Ser residue at position 12 of the protected variant of Fragment 1 is protected as a dimethylated pseudoproline (Psi (Me,Me) pro) moiety in which the oxazolidine is derived from Ser; and
the side chain of the Tyr residue at position 1 of the protected variant of Fragment 2B is protected with a tBu protecting group; the side chains of the Glu residues at position 3, position 4, and position 9 of the protected variant of Fragment 2B are each protected with a OtBu protecting group; the side chain of the Gln residue at position 5 of the protected variant of Fragment 2B is protected with a Trt protecting group; and the side chain of the Lys residue at position 8 of the protected variant of Fragment 2B is protected with a tert-butyloxycarbonyl (Boc) protecting group.

45. The method of any one of claims 42 to 44, wherein:
the protected variant of Fragment 5 is Trt-Ala-Trp(Boc)-Leu-Val-Lys(Boc)-Gly-Gly-Gly-OH (SEQ ID NO: 59);
the protected variant of Fragment 6 is H-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 130);
the protected variant of Fragment 1 is Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-OH (SEQ ID NO: 166); and
the protected variant of Fragment 2B is H-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 30).

46. The method of any one of claims 42 to 45, wherein:
the first protected variant of Peptide B is Trt-Ala-Trp(Boc)-Leu-Val-Lys(Boc)-Gly-Gly-Gly-Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 170);
the second protected variant of Peptide B is H-Ala-Trp(Boc)-Leu-Val-Lys(Boc)-Gly-Gly-Gly-Gly-Gly-Gly-Gly-Ser(Psi(Me,Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 147); and
the protected variant of Peptide D is Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-OH (SEQ ID NO: 169).

47. The method of any one of claims 42 to 46, wherein the protected variant of Peptide A is Boc-His (Trt)-Aib-Glu(OtBu)-Gly-Thr(tBu)-Phe-Thr(tBu)-Ser(tBu)-Asp(OtBu)-Tyr(tBu)-Ser(tBu)-Ser(Psi(Me,Me)Pro)-Tyr(tBu)-Leu-Glu(OtBu)-Glu(OtBu)-Gln(Trt)-Ala-Ala-Lys(Boc)-Glu(OtBu)-Phe-Ile-Ala-Trp(Boc)-Leu-Val-Lys(Boc)-Gly-Gly-Gly-Gly-Gly-Gly-Gly-Ser(Psi(Me, Me)Pro)-Gly-Gly-Gly-Gly-Ser(Psi(Me,Me)Pro)-Gly-Gly-Gly-Gly-Ser(Psi(Me,Me)Pro)-Lys(ivDde)-NH₂ (SEQ ID NO: 171).

48. The method of any one of claims 1 to 47, wherein the method further comprises bromoacetylating an ε-amino group of the C-terminal Lys residue of a protected variant of Peptide A to form a bromoacetylated protected variant of Peptide A, optionally wherein the bromoacetylating comprises selectively deprotecting the side chain of the C-terminal Lys residue of the protected variant of Peptide A to form a partially deprotected variant of Peptide A and then coupling the partially deprotected variant of Peptide A with bromoacetic acid or bromoacetic anhydride to form a bromoacetylated protected variant of Peptide A, wherein the coupling is mediated by a coupling reagent and optionally a coupling additive and/or a base.

49. The method of claim 48, wherein the method further comprises contacting the bromoacetylated protected variant of Peptide A with a Brønsted acid or a Lewis acid, optionally in the presence of a scavenger, to form a bromoacetylated variant of Peptide A, wherein the bromoacetylated variant of Peptide A has the amino acid sequence of SEQ ID NO: 1, the N-terminus and the side chains of the amino acids at positions 1-46 of the bromoacetylated variant of Peptide A are not protected, the C-terminal Lys residue is bromoacetylated, and the C-terminus of the bromoacetylated variant of Peptide A is amidated as a C-terminal primary amide.

50. A molecule selected from:
(SEQ ID NO: 166)
Boc-His(Trt)-Aib-Glu(OtBu)-Gly-Thr(tBu)-Phe-
Thr(tBu)-Ser(tBu)-Asp(OtBu)-Tyr(tBu)-Ser(tBu)-
Ser(Psi(Me,Me)Pro)-OH;
(SEQ ID NO: 30)
H-Tyr(tBu)-Leu-Glu(OtBu)-Glu(OtBu)-Gln(Trt)-
Ala-Ala-Lys(Boc)-Glu(OtBu)-Phe-Ile-OH;
(SEQ ID NO: 169)
Boc-His(Trt)-Aib-Glu(OtBu)-Gly-Thr(tBu)-Phe-
Thr(tBu)-Ser(tBu)-Asp(OtBu)-Tyr(tBu)-Ser(tBu)-
Ser(Psi(Me,Me)Pro)-Tyr(tBu)-Leu-Glu(OtBu)-
Glu(OtBu)-Gln(Trt)-Ala-Ala-Lys(Boc)-Glu(OtBu)-
Phe-Ile-OH;
(SEQ ID NO: 59)
Trt-Ala-Trp(Boc)-Leu-Val-Lys(Boc)-Gly-Gly-Gly-OH;
(SEQ ID NO: 130)
H-Gly-Gly-Gly-Gly-Ser(Psi(Me,Me)Pro)-Gly-Gly-
Gly-Gly-Ser(Psi(Me,Me)Pro)-Gly-Gly-Gly-Gly-
Ser(Psi(Me,Me)Pro)-Lys(ivDde)-NH₂;
(SEQ ID NO: 170)
Trt-Ala-Trp(Boc)-Leu-Val-Lys(Boc)-Gly-Gly-Gly-
Gly-Gly-Gly-Gly-Ser(Psi(Me,Me)Pro)-Gly-Gly-
Gly-Gly-Ser(Psi(Me,Me)Pro)-Gly-Gly-Gly-Gly-
Ser(Psi(Me,Me)Pro)-Lys(ivDde)-NH₂;
(SEQ ID NO: 147)
H-Ala-Trp(Boc)-Leu-Val-Lys(Boc)-Gly-Gly-Gly-
Gly-Gly-Gly-Gly-Ser(Psi(Me,Me)Pro)-Gly-Gly-
Gly-Gly-Ser(Psi(Me,Me)Pro)-Gly-Gly-Gly-Gly-
Ser(Psi(Me,Me)Pro)-Lys(ivDde)-NH₂;
and
(SEQ ID NO: 171)
Boc-His(Trt)-Aib-Glu(OtBu)-Gly-Thr(tBu)-Phe-
Thr(tBu)-Ser(tBu)-Asp(OtBu)-Tyr(tBu)-
Ser(tBu)-Ser(Psi(Me,Me)Pro)-Tyr(tBu)-Leu-
Glu(OtBu)-Glu(OtBu)-Gln(Trt)-Ala-Ala-
Lys(Boc)-Glu(OtBu)-Phe-Ile-Ala-Trp(Boc)-
Leu-Val-Lys(Boc)-Gly-Gly-Gly-Gly-Gly-Gly-
Gly-Ser(Psi(Me,Me)Pro)-Gly-Gly-Gly-Gly-
Ser(Psi(Me,Me)Pro)-Gly-Gly-Gly-Gly-
Ser(Psi(Me,Me)Pro)-Lys(ivDde)-NH₂,
or a salt thereof.
